(19) United States
(12) Patent Application Publication
(10) Pub. No.: US 2023/0028880 A1
(43) Pub. Date: Jan. 26, 2023

(54) CD38-BINDING AGENTS AND USES THEREOF

(71) Applicant: Biohaven Therapeutics Ltd., New Haven, CT (US)

(72) Inventors: Luca RASTELLI, New Haven, CT (US); Matthew Ernest WELSCH, New Haven, CT (US); Anna BUNIN, New Haven, CT (US); Ann Marie K. ROSSI, New Haven, CT (US); Tetyana BERBASOVA, New Haven, CT (US)

(21) Appl. No.: 17/623,238

(22) PCT Filed: Jun. 24, 2020
(86) PCT No.: PCT/US2020/039466

§ 371 Date: Dec. 27, 2021

Related U.S. Application Data
(60) Provisional application No. 62/870,633, filed on Jul. 3, 2019.
(60) Provisional application No. 62/951,765, filed on Dec. 20, 2019.

(51) Int. Cl. A61K 47/68; A61K 35/17; A61K 35/15; A61K 35/16; A61P 35/00

(52) U.S. Cl. CPC A61K 47/6849; A61K 47/6811; A61K 35/17; A61K 35/15; A61K 35/16; A61P 35/00

(57) ABSTRACT

Among other things, the present disclosure provides compounds comprising antibody binding moieties and CD38-binding targeting moieties. In some embodiments, provided compounds recruit various types of antibodies to diseased cells such as cancer cells, and induce immune activities to kill such cells. Provided technologies are useful for treating various diseases including cancer.

### CROSS-REFERENCE TO RELATED APPLICATIONS

This application is a National Stage entry of PCT/2020/039466, filed on Jun. 24, 2020, which claims priority to U.S. Provisional Application No. 62/870,633, filed Jul. 3, 2019, and U.S. Provisional Application No. 62/951,765, filed Dec. 20, 2019, and all the benefits accruing therefrom under 35 U.S.C. § 119, each of which is incorporate by reference herein in its entirety.

### TECHNICAL FIELD

The present disclosure, among other things, provide technologies (e.g., compounds, compositions and methods thereof) useful for, e.g., treating various conditions, disorders or diseases.

### BACKGROUND

Immune system activities may be utilized to prevent or treat various conditions, disorders and diseases.

### SUMMARY

In some embodiments, the present disclosure provides technologies, e.g., compounds, compositions, methods, etc., that are particularly useful for recruiting antibodies to damaged or defective tissues (e.g., tumors, certain wounds, etc.), foreign objects or entities (e.g., infectious agents), etc., which comprises CD38 or fragments thereof. In some embodiments, provided technologies can trigger, generate, encourage, and/or enhance immune system activities toward target cells, tissues, objects and/or entities which express CD38, for example, antibody-dependent cell-mediated cytotoxicity (ADCC), antibody-dependent cellular phagocytosis (ADCP), etc. In some embodiments, the present disclosure is directed to design, preparation, and use of molecules capable of redirecting endogenous antibodies selectively to diseased cells, e.g., cancer cells, which express CD38, and inducing immune system activities, e.g., an antibody-directed, cell-mediated immune response, e.g., cytotoxicity, ADCP, etc.

In some embodiments, the present disclosure provides antibody recruiting molecules (ARMs), which comprise antibody binding moieties, target binding moieties (e.g., those binding to CD38) and optionally linker moieties. In some embodiments, target binding moieties confer specificity of ARMs to their target, e.g., a diseased cell of interest, through, e.g., binding of an entity differentiating a target from a non-target (e.g., diseased cells from other cell types). Among other things, ARMs can enable target-specific recruitment of antibodies, e.g., endogenous antibodies, administered antibodies, etc., through ABTs, and/or trigger, generate, encourage, and/or enhance immune activities, e.g., immune-mediated killing of target cells. In some embodiments, provided technologies comprise ARMs that comprise CD38-binding target binding moieties, and can selectively recruit antibodies to CD38-expressing targets such as cancer cells, and/or trigger, generate, encourage, and/or enhance immune activities (e.g., ADCC, ADCP, etc.) toward such target cells. In some embodiments, CD-expressing target cells are cancer cells. In some embodiments, provided agents herein, e.g., CD38-binding ARMs, are particularly useful for preventing and/or treating conditions, disorders or diseases associated with CD38, e.g., various types of cancers associated with CD38.

Among other things, the present disclosure encompasses the recognition that certain immunotherapies targeting CD38-expressing targets, such as CD38 antibodies, suffer from one or more side effects (e.g., toxicities) due to, without the intention to be limited by any particular theory, reduction and/or depletion of normal cells expressing CD38. For example, as described herein, in some embodiments, CD38 antibodies (e.g., Daratumumab) induced reduction or depletion of CD38-expressing immune effector cells. Among other things, the present disclosure demonstrates the provided technologies can recruit immune components and activities to CD38-expressing targets, e.g., cancer cells, with less, or no significant, reduction or depletion of CD38-expressing immune effector cells compared to CD38 antibodies such as Daratumumab.

In some embodiments, a provided compound, e.g., an ARM, comprises antibody binding moieties that can bind to antibodies of various specificity (universal antibody-binding termini, uABTs). Among other things, such ARMs can circumvent the dependence of specific antibody populations and/or undesirable effects that are associated with individual variations of specific antibody populations. In some embodiments, uABTs can bind to Fc region of antibodies and thereby can, among other things, recruit antibodies of various antigen-specificity. In some embodiments, ABTs, e.g., uABTs, bind to a conserved site present in the Fc region of IgG. In some embodiments, uABTs enables recruitment of all IgG subclasses (IgG1, IgG2, IgG3, IgG4). In some embodiments, uABTs enables recruitment preferentially of IgG1, IgG2, and/or IgG4. In some embodiments, uABTs bind to IgG molecules and not human IgA or IgM. In some embodiments, recruitment of antibodies, e.g., IgG subclasses, is dependent on the administered dose of an ARM, and/or is not by levels of antibodies having a particular Fab region in an individual. In some embodiments, a useful ABT is one described in WO 2019/023501, whose antibody binding moieties, e.g., various ABTs including uABTs, are incorporated herein by reference. Those skilled in the art will appreciate that various antibody binding moieties are available and can be utilized in accordance with the present disclosure.

Among other things, ARMs can recruit antibodies and the antibodies recruited provide one or more immune activities, e.g., through one or more antibody-mediated immune mechanisms. In some embodiments, recruited antibodies recruit immune cells and/or interact and/or activates Fc receptors of immune cells. In some embodiments, recruited antibodies recruits and activates immune cells and inhibit and/or target diseased cells such as cancer cells. In some embodiments, provided agents, e.g., ARMs, induce antibody-dependent effector functions. In some embodiments, provided agents, e.g., ARMs, induce complement dependent cytotoxicity (CDC). In some embodiments, provided agents, e.g., ARMS, induce direct cytotoxicity. In some embodiments, provided agents, e.g., ARMs, inhibit biological functions associated with steric blockade. In some embodiments, provided agents, e.g., ARMs, induce antibody-dependent cell-mediated virus inhibition (ADCVI). In some embodiments, provided agents, e.g., ARMs, induce ADCC and kill cancer cells. In some embodiments, provided agents, e.g., ARMs, induce ADCP and kill cancer cells. In some embodiments, provided agents, e.g., ARMs, induce both ADCC and ADCP.

In some embodiments, the present disclosure provide an agent comprising:

an antibody binding moiety,

a target binding moiety, and

optionally a linker moiety.

In some embodiments, a target binding moiety can bind CD38. In some embodiments, an antibody binding moiety can bind to two or more antibodies which have different Fab regions. In some embodiments, an antibody binding moiety can bind to two or more antibodies which have different antigen specificity. In some embodiments, an antibody binding moiety can bind to Fc regions of various antibodies. In some embodiments, an antibody binding moiety, e.g., a universal antibody binding moiety, binds to an Fc region of an antibody. In some embodiments, an antibody binding moiety, e.g., a universal antibody binding moiety, binds to a conserved Fc region of an antibody. In some embodiments, an antibody binding moiety binds to an Fc region of an IgG antibody. In some embodiments, upon binding with an antibody binding moiety (e.g., at a Fc region), an antibody can still perform all, or substantially all, or most of its biological functions. For example, an antibody upon binding with an antibody binding moiety can recruit and/or activate immune cells, e.g., through interactions with various Fc receptors.

In some embodiments, the present disclosure provides compounds that have the general formula I:

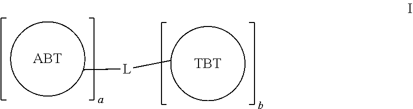

or a pharmaceutically acceptable salt thereof, wherein each variable is independently as defined and described herein. In some embodiments, a provided agent is a compound of formula I or a salt thereof.

In some embodiments, a provided agent, e.g., a compound of formula I, is a compound having the structure of

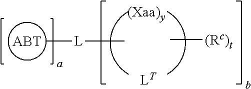

or a pharmaceutically acceptable salt thereof, wherein each variable is as defined and described in the present disclosure.

In some embodiments, a provided agent is a compound of formula I-a or a salt thereof. In some embodiments, the present disclosure provides a compound of formula I-a:

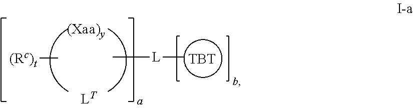

or a pharmaceutically acceptable salt thereof, wherein each variable is as defined and described in the present disclosure. In some embodiments, a provided compound of formula I is a compound of formula I-a.

In some embodiments, a provided agent is a compound of formula I-b or a salt thereof. In some embodiments, the present disclosure provides a compound of formula I-b:

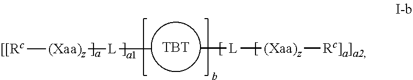

or a pharmaceutically acceptable salt thereof, wherein each variable is as defined and described in the present disclosure. In some embodiments, a provided compound of formula I is a compound of formula I-b.

In some embodiments, provided agents and compounds of the present disclosure, and pharmaceutically acceptable compositions thereof, are effective for recruiting antibodies to diseased cells, e.g., cancer cells. In some embodiments, the present disclosure provides compounds that have the general formula II:

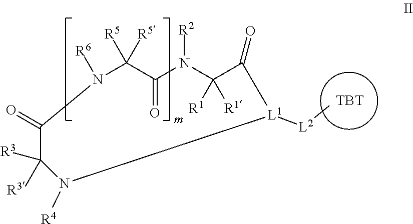

or a pharmaceutically acceptable salt thereof, wherein each variable is as defined and described herein. In some embodiments, a provided agent is a compound of formula II or a salt thereof. In some embodiments, a provided compound of formula I is a provided compound of formula II or a salt thereof. In some embodiments, a compound having the structure of formula I-a is a compound of formula II.

In some embodiments, the present disclosure provides compounds that have the general formula III:

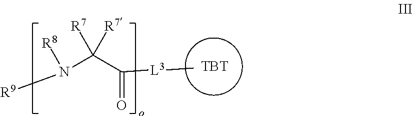

or a pharmaceutically acceptable salt thereof, wherein each variable is as defined and described herein. In some embodiments, a provided agent is a compound of formula III or a salt thereof. In some embodiments, a provided compound of formula I is a provided compound of formula III or a salt thereof. In some embodiments, a compound having the structure of formula I-b is a compound of formula III.

Compounds of the present disclosure, and pharmaceutically acceptable compositions thereof, are useful for treating a variety of diseases, disorders or conditions. Such diseases, disorders, or conditions include those described herein. In some embodiments, a condition, disorder or disease is cancer.

### BRIEF DESCRIPTION OF THE DRAWINGS

FIG. 1. Provided compounds can recruit antibodies to target cells.

FIG. 2. Provided compounds can recruit antibodies to target cells and activate effector cells.

FIG. 3. Provided compounds can kill target cells.

FIG. 4. Provided compounds do not significantly deplete effector cells. Daratumumab 3 ug/mL-0.1 ug/mL. I-9 300 nM-10 nM. IvIG 10 ug/mL.

FIG. 5. Provided technologies have low toxicity. A. Frequencies of dead NK cells. B. Frequencies of dead NK cells normalized to DMSO-treated control. CD38 ARM I-17.

FIG. 6. Provided technologies can effectively kill cancer cells. A. Frequencies of dead SUDHL-4 cells in NK-SUDHL-4 co-cultures. B. Frequencies of dead SUDHL-4 cells in NK-SUDHL-4 co-cultures, normalized to DMSO treated controls. CD38 ARM is I-17.

FIG. 7. Provided technologies can reduce the number of plasma cells. CD38 ARM is I-17.

FIG. 8. No or low level of undesired NK cell fratricide by provided technologies. CD38 ARM is I-17.

FIG. 9. Provided technologies can effectively reduce the number of target cells. Enumeration of CD38 expressing Daudi cells in intraperitoneal cavity of SCID mice as a readout of, without the intention to be limited by theory, macrophage-mediated I-17 dependent phagocytosis. From left to right: control; IVIG 10 mg/mouse sub-Q; I-17 1 mg/kg+IVIG (10 mg/mouse sub-Q); I-17 10 mg/kg+IVIG (10 mg/mouse sub-Q); I-17 30 mg/kg+IVIG (10 mg/mouse sub-Q); Daratumumab. ***: P<0.001.

FIG. 10. Activity of CIML NK cells frozen in combination with I-17 and recovered against MOLP-8 cells. From left to right: untreated; daratumumab; cryopreserved CIML NK I-17 added to assay; and CIML NK cryopreserved with I-17.

### DETAILED DESCRIPTION OF CERTAIN EMBODIMENTS

### 1. General Description of Certain Embodiments

In some embodiments, the present disclosure provides agents, e.g., ARMs, that comprise target binding moieties that can bind to CD38. In some embodiments, provided agents, e.g., ARMs, comprise universal antibody binding moieties that can bind to antibodies with different Fab structures. In some embodiments, the present disclosure provides agents, e.g., ARMs, that comprises antibody binding moieties that bind to antibodies, e.g., Fc regions of antibodies, and such binding of antibodies do not interfere one or more immune activities of the antibodies, e.g., interaction with Fc receptors (e.g., CD16a), recruitment of effector cells like NK cells (e.g., for ADCC), macrophage (e.g., for ADCP), etc. As those skilled in the art will appreciate, provided technologies (agents, compounds, compositions, methods, etc.) of the present disclosure can provide various advantages, for example, provided technologies can utilize antibodies having various Fab regions in the immune system to avoid or minimize undesired effects of antibody variations among a patient population, can trigger, and/or enhance, immune activities toward targets, e.g., killing target diseased cells such as cancer cells, and/or are of low toxicities compared to certain antibody therapeutics (e.g., low complement activation, significantly less reduction of CD38-expressing normal cells (e.g., effector cells).

In some embodiments, provided technologies are useful for modulating immune activities, such as ADCC, ADCP, and combinations thereof against targets (diseased cells, foreign objects or entities, etc.) comprising CD38. In some embodiments, technologies of the present disclosure are useful for recruiting antibodies to cancer cells, particularly those expressing CD38. In some embodiments, provided technologies are useful for modulating ADCC against target cells, e.g., diseased cells such as cancer cells. In some embodiments, provided technologies are useful for modulating ADCP against target cells, e.g., diseased cells such as cancer cells. In some embodiments, provided agents can inhibit protein activities. In some embodiments, a target binding moiety is an inhibitor moiety. In some embodiments, a target binding moiety is an enzyme inhibitor moiety.

In some embodiments, the present disclosure provide an agent comprising:

an antibody binding moiety,

a target binding moiety which can bind CD38, and

optionally a linker moiety,

wherein the antibody binding moiety can bind to two or more antibodies which have different Fab regions.

In some embodiments, the present disclosure provide an agent comprising:

an antibody binding moiety,

a target binding moiety which can bind CD38, and

optionally a linker moiety,

wherein the antibody binding moiety can bind to two or more antibodies which have different Fab regions.

In some embodiments, provided agents comprise two or more antibody binding moieties. In some embodiments, provided agents comprise two or more target binding moieties.

An antibody binding moiety may interact with any portion of an antibody. In some embodiments, an antibody binding moiety binds to an Fc region of an antibody. In some embodiments, an antibody binding moiety binds to a conserved Fc region of an antibody. In some embodiments, an antibody binding moiety binds to an Fc region of an IgG antibody. As appreciated by those skilled in the art, various antibody binding moieties, linkers, and target binding moieties can be utilized in accordance with the present disclosure. Among other things, as demonstrated in the Examples, in some embodiments, the present disclosure provides antibody binding moieties, linkers, and target binding moieties and combinations thereof that are particularly useful and effective for constructing ARM molecules to recruit antibodies to target cells, and/or to trigger, generate, encourage, and/or enhance immune system activities toward target cells, e.g., diseased cells such as cancer cells.

In some embodiments, the present disclosure provides antibody binding moieties and/or agents (e.g., compounds of various formulae described in the present disclosure, ARM molecules of the present disclosure, etc.) comprising antibody binding moieties that can bind to a Fc region that is bound to Fc receptors, e.g., FcγRIIIa, CD16a, etc. In some embodiments, provided moieties and/or agents comprising antibody binding moieties that bind to a complex comprising an Fc region and an Fc receptor. In some embodiments, the present disclosure provides a complex comprising:

an agent comprising:
an antibody binding moiety,
a target binding moiety, and
optionally a linker moiety,

an Fc region, and

an Fc receptor.

In some embodiments, an antibody binding moiety can bind to CD38, and/or an antibody binding moiety of the agent can bind to two or more antibodies which have different Fab regions.

In some embodiments, an Fc region is an Fc region of an endogenous antibody of a subject. In some embodiments, an Fc region is an Fc region of an exogenous antibody. In some embodiments, an Fc region is an Fc region of an administered agent. In some embodiments, an Fc receptor is of a diseased cell in a subject. In some embodiments, an Fc receptor is of a cancer cell in a subject.

In certain embodiments, the present disclosure provides a compound of formula I:

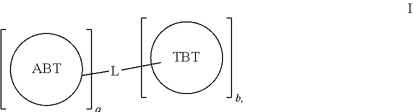

or a pharmaceutically acceptable salt thereof, wherein:

each of a and b is independently 1-200;

each ABT is independently an antibody binding moiety;

L is a bivalent or multivalent linker moiety that connects ABT with TBT; and

each TBT is independently a target binding moiety.

In some embodiments, ABT is a universal antibody binding moiety.

In some embodiments, an antibody binding moiety comprises one or more amino acid residues. In some embodiments, an antibody binding moiety is or comprises a peptide moiety. In some embodiments, an antibody binding moiety is or comprises a cyclic peptide moiety. In some embodiments, such antibody binding moiety comprises one or more natural amino acid residues. In some embodiments, such antibody binding moiety comprises one or more unnatural natural amino acid residues.

In some embodiments, an amino acid has the structure of formula A-I:

NH(R^{a1})-L^{a1}-C(R^{a2})(R^{a3})-L^{a2}-COOH, A-I

or a salt thereof, wherein:

each of R^{a1}, R^{a2}, R^{a3} is independently -L^{a}-R′;

each of L^{a1} and L^{a2} is independently L^{a};

each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;

each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;

each R′ is independently —R, —C(O)R, —CO₂R, or —SO₂R;

each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, an amino acid analog is a compound in which the amino group and/or carboxylic acid group are independently replaced with an optionally substituted aliphatic or heteroaliphatic moiety. As those skilled in the art will appreciate, many amino acid analogs, which mimics structures, properties and/or functions of amino acids, are described in the art and can be utilized in accordance with the present disclosure.

In some embodiments, an antibody-binding moiety is a cyclic peptide moiety. In some embodiments, the present disclosure provides a compound of formula I-a:

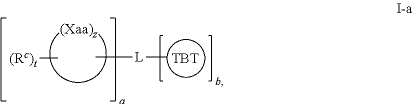

or a salt thereof, wherein:

each Xaa is independently a residue of an amino acid or an amino acid analog;

t is 0-50;

z is 1-50;

L is a linker moiety;

TBT is a target binding moiety;

each R^{c} is independently -L^{a}-R′;

each of a and b is independently 1-200;

each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;

each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;

each R′ is independently —R, —C(O)R, —CO₂R, or —SO₂R;

each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, a is 1. In some embodiments, b is 1. In some embodiments, a is 1 and b is 1, and a compound of formula I-a has the structure of

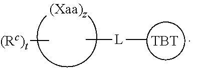

In some embodiments, each residue, e.g., Xaa, is independently a residue of an amino acid or an amino acid analog, wherein the amino acid or the amino acid analog has the structure of H-L^{a1}-L^{a1}-C(R^{a2})(R^{a3})-L^{a2}-L^{a2}-H or a salt thereof. In some embodiments, an amino acid has the structure of NH(R^{a1})-L^{a1}-C(R^{a2})(R^{a3})-L^{a2}-COOH or a salt thereof. In some embodiments, an amino acid analog has the structure of H-L^{a1}-L^{a1}-C(R^{a2})(R^{a3})-L^{a2}-L^{a2}-H or a salt thereof. In some embodiments, in such an amino acid analog, the first -L^{a1}- (bonded to —H in the formula) is not —N(R^{a1})— (e.g., is optionally substituted bivalent C₁₋₆ aliphatic). In some embodiments, in H-L^{a1}-L^{a1}-, -L^{a1}-L^{a1}- bonds to the —H through an atom that is not nitrogen. In some embodiments, in -L^{a2}-L^{a2}-H, -L^{a2}-L^{a2}- is not bonded to the —H through —C(O)O—. In some embodiments, each residue, e.g., each Xaa in formula I-a, is independently a residue of an amino acid having the structure of formula A-I.

In some embodiments, each Xaa independently has the structure of -L^{a1}-L^{a1}-C(R^{a2})(R^{a3})-L^{a2}-L^{a2}-. In some embodiments, each Xaa independently has the structure of -L^{aX1}-L^{a1}-C(R^{a2})(R^{a3})-L^{a2}-L^{aX2}- wherein L^{aX1} is optionally substituted —NH—, optionally substituted —CH₂—, —N(R^{a1})—, or —S—, L^{aX2} is optionally substituted —NH—, optionally substituted —CH₂—, —N(R^{a1})—, or —S—, and each other variable is independently as described herein. In some embodiments, L^{aX1} is optionally substituted —NH—, or —N(R^{a1})—. In some embodiments, L^{aX1} is optionally substituted —CH₂—, or —S—. In some embodiments, L^{aX2} is optionally substituted —NH—, optionally substituted —CH₂—, —N(R^{a1})—, or —S—. In some embodiments, optionally substituted —CH₂— is —C(O)—. In some embodiments, optionally substituted —CH₂— is not —C(O)—. In some embodiments, L^{aX2} is —C(O)—. In some embodiments, each Xaa independently has the structure of —N(R^{a1})-L^{a1}-C(R^{a2})(R^{a3})-L^{a2}-CO—.

In many embodiments, two or more residues, e.g., two or more Xaa residues, are linked together such that one or more cyclic structures are formed. For example, various compounds in Table 1 comprises linked residues. Residues can be linked, optionally through a linker (e.g., L^{T}) at any suitable positions. For example, a linkage between two residues can connect each residue independently at its N-terminus, C-terminus, a point on the backbone, or a point on a side chain, etc. In some embodiments, two or more side chains of residues, e.g., in compounds of formula I-a, (e.g., R^{a2} or R^{a3} of one amino acid residue with R^{a2} or R^{a3} of another amino acid residue) are optionally take together to form a bridge (e.g., in various compounds in Table 1, etc.), e.g., in some embodiments, two cysteine residues form a —S—S— bridge as typically observed in natural proteins. In some embodiments, a formed bridge has the structure of L^{b}, wherein L^{b} is L^{a} as described in the present disclosure. In some embodiments, each end of L^{b} independently connects to a backbone atom of a cyclic peptide (e.g., a ring atom of the ring formed by -(Xaa)_{z}- in formula I-a). In some embodiments, L^{b} comprises an R group (e.g., when a methylene unit of L^{b} is replaced with —C(R)₂— or —N(R)—), wherein the R group is taken together with an R group attached to a backbone atom (e.g., R^{a1}, R^{a2}, R^{a3}, etc. if being R) and their intervening atoms to form a ring. In some embodiments, L^{b} connects to a ring, e.g., the ring formed by -(Xaa)_{z}- in formula I-a through a side chain of an amino acid residue (e.g., Xaa in formula I-a). In some embodiments, such a side chain comprises an amino group or a carboxylic acid group. In some embodiments, L^{T} is L^{b} as described herein. In some embodiments, a linkage, e.g., L^{b} or L^{T}, connects a side chain with a N-terminus or a C-terminus of a residue. In some embodiments, a linkage connects a side chain with an amino group of a residue. In some embodiments, a linkage connects a side chain with an alpha-amino group of a residue. In some embodiments, as illustrated herein, a linkage, e.g., L^{b} or L^{T}, is —CH₂—C(O)—. In some embodiments, the —CH₂— is bonded to a side chain, e.g., boned to —S— of a cysteine residue, and the —C(O)— is bonded to an amino group, e.g., an alpha-amino group of a residue. In some embodiments, a linkage, e.g., L^{b} or L^{T}, is optionally substituted —CH₂—S—CH₂—C(O)—NH—, wherein each end is bonded to the alpha-carbon of a residue. In some embodiments, the —NH— is of an alpha-amino group of a residue, e.g., of a N-terminal residue.

In some embodiments,

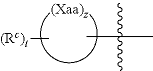

is an antibody binding moiety

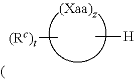

binds to an antibody). In some embodiments,

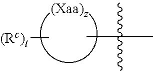

is a universal antibody binding moiety. In some embodiments,

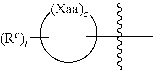

is a universal antibody binding moiety which can bind to antibodies having different Fab regions. In some embodiments,

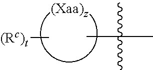

is a universal antibody binding moiety that can bind to a Fc region. In some embodiments, an antibody binding moiety, e.g., a universal antibody binding moiety having the structure of

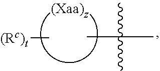

can bind to a Fc region bound to an Fc receptor. In some embodiments, an antibody binding moiety, e.g., of an antibody binding moiety having the structure of

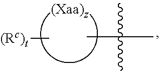

has the structure of

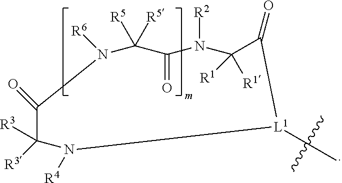

In some embodiments,

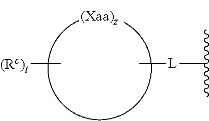

has the structure of

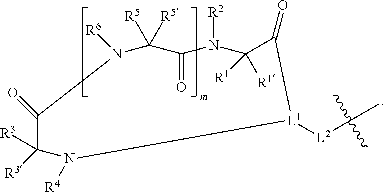

In certain embodiments, the present disclosure provides a compound of formula II:

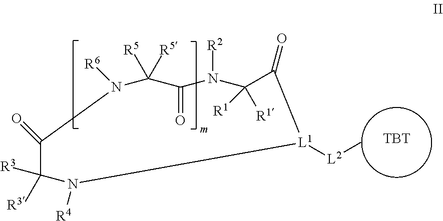

or a pharmaceutically acceptable salt thereof, wherein:
each of R¹, R³ and R⁵ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or:
   R¹ and R^{1′} are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur;
   R³ and R^{3′} are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur;
   an R⁵ group and the R^{5′} group attached to the same carbon atom are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur; or
   two R⁵ groups are optionally taken together with their intervening atoms to form a C₁₋₁₀ optionally substituted bivalent straight or branched saturated or unsaturated hydrocarbon chain wherein 1-3 methylene units of the chain are independently and optionally replaced with —S—, —SS—, —N(R)—, —O—, —C(O)—, —OC(O)—, —C(O)O—, —C(O)N(R)—, —N(R)C(O)—, —S(O)—, —S(O)₂—, or -Cy¹-, wherein each -Cy¹- is independently a 5-6 membered heteroarylenyl with 1-4 heteroatoms independently selected from nitrogen, oxygen or sulfur;
each of R^{1′}, R^{3′} and R^{5′} is independently hydrogen or optionally substituted C₁₋₃ aliphatic;
each of R², R⁴ and R⁶ is independently hydrogen, or optionally substituted C₁₋₄ aliphatic, or:
   R² and R¹ are optionally taken together with their intervening atoms to form a 4-8 membered, optionally substituted saturated or partially unsaturated monocyclic heterocyclic ring having 1-2 heteroatoms independently selected from nitrogen, oxygen, or sulfur;
   R⁴ and R³ are optionally taken together with their intervening atoms to form a 4-8 membered optionally substituted saturated or partially unsaturated monocyclic heterocyclic ring having 1-2 heteroatoms independently selected from nitrogen, oxygen, or sulfur; or
   an R⁶ group and its adjacent R⁵ group are optionally taken together with their intervening atoms to form a 4-8 membered optionally substituted saturated or partially unsaturated monocyclic heterocyclic ring having 1-2 heteroatoms independently selected from nitrogen, oxygen, or sulfur;
L¹ is a trivalent linker moiety that connects

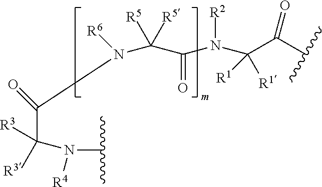

and

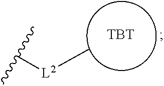

L² is a covalent bond or a C₁₋₃₀ optionally substituted bivalent straight or branched saturated or unsaturated hydrocarbon chain wherein 1-10 methylene units of the chain are independently and optionally replaced with —S—, —N(R)—, —O—, —C(O)—, —OC(O)—, —C(O)O—, —C(O)N(R)—, —N(R)C(O)—, —S(O)—, —S(O)₂—,

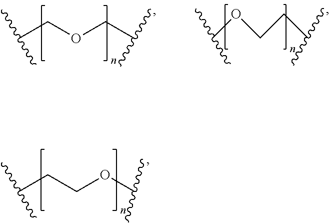

or -Cy¹-, wherein each -Cy¹- is independently a 5-6 membered heteroarylenyl with 1-4 heteroatoms independently selected from nitrogen, oxygen or sulfur;
TBT is a target binding moiety; and
each of m and n is independently 1, 2, 3, 4, 5, 6, 7, 8, 9, or 10.

In some embodiments, an antibody binding moiety is or comprises a peptide moiety. In some embodiments, the present disclosure provides a compound having the structure of formula I-b:

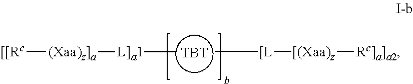

or a salt thereof, wherein:

each Xaa is independently a residue of an amino acid or an amino acid analog;

each z is independently 1-50;

each L is independently a linker moiety;

TBT is a target binding moiety,

each R^{c} is independently -L^{a}-R′;

each of a1 and a2 is independently 0 or 1, wherein at least one of a1 and a2 is not 0;

each of a and b is independently 1-200;

each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;

each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;

each R′ is independently —R, —C(O)R, —CO₂R, or —SO₂R;

each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, a1 is 1. In some embodiments, a2 is 1. In some embodiments, b is 1. In some embodiments, a compound of formula I-b has the structure of

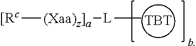

In some embodiments, a compound of formula I-b has the structure of

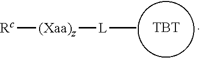

In some embodiments, a compound of formula I-b has the structure of

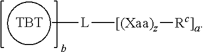

In some embodiments, a compound of formula I-b has the structure of

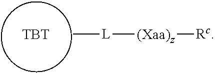

In some embodiments, each residue, e.g., each Xaa in formula I-a, I-b, etc., is independently a residue of amino acid having the structure of formula A-I. In some embodiments, each Xaa independently has the structure of —N(R^{a1})-L^{a1}-C(R^{a2})(R^{a3})-L^{a2}-CO—. In some embodiments, two or more side chains of the amino acid residues, e.g., in compounds of formula I-a, (e.g., R² or R^{a3} of one amino acid residue with R² or R^{a3} of another amino acid residue) are optionally take together to form a bridge (e.g., various compounds in Table 1), e.g., in some embodiments, two cysteine residues form a —S—S— bridge as typically observed in natural proteins. In some embodiments, a formed bridge has the structure of L^{b}, wherein L^{b} is L^{a} as described in the present disclosure. In some embodiments, each end of L^{b} independently connects to a backbone atom of a cyclic peptide (e.g., a ring atom of the ring formed by -(Xaa)_{z}- in formula I-a). In some embodiments, L^{b} comprises an R group (e.g., when a methylene unit of L^{b} is replaced with —C(R)₂— or —N(R)—), wherein the R group is taken together with an R group attached to a backbone atom (e.g., R^{a1}, R^{a2}, R^{a3}, etc. if being R) and their intervening atoms to form a ring. In some embodiments, L^{b} connects to a ring, e.g., the ring formed by -(Xaa)_{z}- in formula I-b through a side chain of an amino acid residue (e.g., Xaa in formula I-a). In some embodiments, such a side chain comprises an amino group or a carboxylic acid group.

In some embodiments, R^{c}-(Xaa)z- is an antibody binding moiety (R^{c}-(Xaa)z-H binds to an antibody). In some embodiments, R^{c}-(Xaa)z- is a universal antibody binding moiety. In some embodiments, R^{c}-(Xaa)z- is a universal antibody binding moiety which can bind to antibodies having different Fab regions. In some embodiments, R^{c}-(Xaa)z- is a universal antibody binding moiety that can bind to a Fc region. In some embodiments, an antibody binding moiety, e.g., a universal antibody binding moiety having the structure of R^{c}-(Xaa)z-, can bind to a Fc region which binds to an Fc receptor. In some embodiments, R^{c}-(Xaa)z- has the structure of

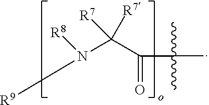

In some embodiments, R^{c}-(Xaa)z-L- has the structure of

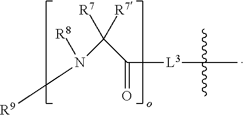

In certain embodiments, the present disclosure provides a compound of formula III:

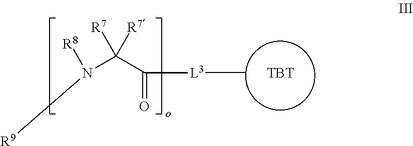

or a pharmaceutically acceptable salt thereof, wherein:
each of R⁷ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or:
   an R⁷ group and the R^{7′} group attached to the same carbon atom are optionally taken together with their intervening carbon atom to form a 3-8 membered optionally substituted saturated or partially unsaturated spirocyclic carbocyclic ring or a 3-8 membered optionally substituted saturated or partially unsaturated spirocyclic heterocyclic ring having 1-2 heteroatoms independently selected from nitrogen, oxygen, or sulfur;
each of R^{7′} is independently hydrogen or optionally substituted C₁₋₃ aliphatic;
each of R⁸ is independently hydrogen, or optionally substituted C₁₋₄ aliphatic, or:
   an R⁸ group and its adjacent R⁷ group are optionally taken together with their intervening atoms to form a 4-8 membered optionally substituted saturated or partially unsaturated monocyclic heterocyclic ring having 1-2 heteroatoms independently selected from nitrogen, oxygen, or sulfur;
R⁹ is hydrogen, optionally substituted C₁₋₃ aliphatic, or —C(O)-(optionally substituted C₁₋₃ aliphatic);
L³ is a bivalent linker moiety that connects

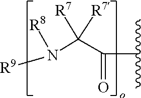

with TBT;
TBT is a target binding moiety; and
o is 1, 2, 3, 4, 5, 6, 7, 8, 9, or 10.

### 2. Definitions

Compounds of the present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included. Unless otherwise specified, compounds described herein may be provided and/or utilized in a salt form, particularly a pharmaceutically acceptable salt form.

Aliphatic: As used herein, “aliphatic” means a straight-chain (i.e., unbranched) or branched, substituted or unsubstituted hydrocarbon chain that is completely saturated or that contains one or more units of unsaturation, or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, an alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Alkynyl: As used herein, the term “alkynyl” refers to an aliphatic group, as defined herein, having one or more triple bonds.

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, an aryl group is a biaryl group. The term “aryl” may be used interchangeably with the term “aryl ring.” In certain embodiments of the present disclosure, “aryl” refers to an aromatic ring system which includes, but is not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

Cycloaliphatic: The term “cycloaliphatic,” “carbocycle,” “carbocyclyl,” “carbocyclic radical,” and “carbocyclic ring,” are used interchangeably, and as used herein, refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, a cycloaliphatic group has 3-6 carbons. In some embodiments, a cycloaliphatic group is saturated and is cycloalkyl. The term “cycloaliphatic” may also include aliphatic rings that are fused to one or more aromatic or nonaromatic rings, such as decahydronaphthyl or tetrahydronaphthyl. In some embodiments, a cycloaliphatic group is bicyclic. In some embodiments, a cycloaliphatic group is tricyclic. In some embodiments, a cycloaliphatic group is polycyclic. In some embodiments, “cycloaliphatic” refers to C₃-C₆ monocyclic hydrocarbon, or C₈-C₁₀ bicyclic or polycyclic hydrocarbon, that is completely saturated or that contains one or more units of unsaturation, but which is not aromatic, that has a single point of attachment to the rest of the molecule, or a C₉-C₁₆ polycyclic hydrocarbon that is completely saturated or that contains one or more units of unsaturation, but which is not aromatic, that has a single point of attachment to the rest of the molecule.

Dosing regimen: As used herein, a “dosing regimen” or “therapeutic regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regime comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount.

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In some embodiments, one or more units selected from C, CH, CH₂, and CH₃ are independently replaced by one or more heteroatoms (including oxidized and/or substituted forms thereof). In some embodiments, a heteroaliphatic group is heteroalkyl. In some embodiments, a heteroaliphatic group is heteroalkenyl.

Heteroalkyl: The term “heteroalkyl”, as used herein, is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-”, as used herein, used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic or polycyclic), in some embodiments 5, 6, 9, or 10 ring atoms. In some embodiments, a heteroaryl group has 6, 10, or 14 71 electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom”, as used herein, means an atom that is not carbon or hydrogen. In some embodiments, a heteroatom is boron, oxygen, sulfur, nitrogen, phosphorus, or silicon (including various forms of such atoms, such as oxidized forms (e.g., of nitrogen, sulfur, phosphorus, or silicon), quaternized form of a basic nitrogen or a substitutable nitrogen of a heterocyclic ring (for example, N as in 3,4-dihydro-2H-pyrrolyl), NH (as in pyrrolidinyl) or NR⁺ (as in N-substituted pyrrolidinyl) etc.). In some embodiments, a heteroatom is oxygen, sulfur or nitrogen.

Heterocycle: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Lower alkyl: The term “lower alkyl” refers to a C₁₋₄ straight or branched alkyl group. Example lower alkyl groups are methyl, ethyl, propyl, isopropyl, butyl, isobutyl, and tert-butyl.

Lower haloalkyl: The term “lower haloalkyl” refers to a C₁₋₄ straight or branched alkyl group that is substituted with one or more halogen atoms.

Optionally Substituted: As described herein, compounds of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In some embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Certain substituents are described below.

Suitable monovalent substituents on a substitutable atom, e.g., a suitable carbon atom, are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —Si(R^{∘})₃; —OSi(R^{∘})₃; —B(R^{∘})₂; —OB(R^{∘})₂; —OB(OR^{∘})₂; —P(R^{∘})₂; —P(OR^{∘})₂; —P(R^{∘})(OR^{∘}); —OP(R^{∘})₂; —OP(OR^{∘})₂; —OP(R^{∘})(OR^{∘}); —P(O)(R^{∘})₂; —P(O)(OR^{∘})₂; —OP(O)(R^{∘})₂; —OP(O)(OR^{∘})₂; —OP(O)(OR^{∘})(SR^{∘}); —SP(O)(R^{∘})₂; —SP(O)(OR^{∘})₂; —N(R^{∘})P(O)(R^{∘})₂; —N(R^{∘})P(O)(OR^{∘})₂; —P(R^{∘})₂[B(R^{∘})₃]; —P(OR^{∘})₂[B(R^{∘})₃]; —OP(R^{∘})₂[B(R^{∘})₃]; —OP(OR^{∘})₂[B(R^{∘})₃]; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined herein and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH₂)₀₋₁(C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{∘}, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)_{0- 2}NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents, e.g., on a suitable carbon atom, are independently the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5-6-membered saturated, partially unsaturated, and aryl ring having 0-4 heteroatoms independently selected from nitrogen, oxygen, and sulfur.

Suitable substituents on the aliphatic group of R* are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

In some embodiments, suitable substituents on a substitutable nitrogen are independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises more than one acid groups. In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations.

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. June 2012, the entirety of Chapter 2 is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, tbutoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifiuoroacetyl, pivaloyl, 9-fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-yl (MOX). In some embodiments, each of the hydroxyl protecting groups is, independently selected from acetyl, benzyl, t-butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group. In some embodiments, a phosphorous linkage protecting group is a group attached to the phosphorous linkage (e.g., an internucleotidic linkage) throughout oligonucleotide synthesis. In some embodiments, a protecting group is attached to a sulfur atom of an phosphorothioate group. In some embodiments, a protecting group is attached to an oxygen atom of an internucleotide phosphorothioate linkage. In some embodiments, a protecting group is attached to an oxygen atom of the internucleotide phosphate linkage. In some embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, or 4-[N-methyl-N-(2,2,2-trifluoroacetyl)amino]butyl.

Subject: As used herein, the term “subject” refers to any organism to which a compound or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In some embodiments, a subject is a human. In some embodiments, a subject may be suffering from and/or susceptible to a disease, disorder and/or condition.

Substantially: As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition is predisposed to have that disease, disorder and/or condition. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the term “therapeutic agent” in general refers to any agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, a therapeutic agent is a compound described herein.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unit dose: The expression “unit dose” as used herein refers to an amount administered as a single dose and/or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included as described infra. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

Unsaturated: The term “unsaturated,” as used herein, means that a moiety has one or more units of unsaturation.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the present disclosure. Unless otherwise stated, all tautomeric forms of the compounds are within the scope of the present disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of the present disclosure. Such compounds are useful, for example, as analytical tools, as probes in biological assays, or as therapeutic agents in accordance with the present disclosure. As appreciated by those skilled in the art, provided compounds, agents, etc. may be provided as solvate thereof.

### 3. Description of Exemplary Embodiments

In some embodiments, the present disclosure provide an agent comprising:

an antibody binding moiety,

a target binding moiety, and

optionally a linker moiety.

In some embodiments, an antibody binding moiety is a uABT. In some embodiments, a target binding moiety can bind to CD38. In some embodiments, an agent is a compound of formula I, I-a, I-b, II or III, or a salt thereof. In some embodiments, the present disclosure provides compounds of formula I, I-a, I-b, II or III, or pharmaceutically acceptable salts thereof. Various embodiments of provided technologies are described herein as examples.

### Antibody Binding Moieties

Among other things, the present disclosure provides agents, e.g., ARMs, comprising antibody binding moieties. In some embodiments, antibody binding moieties are universal antibody binding moieties which can bind to antibodies having different Fab regions and different specificity. In some embodiments, antibody binding moieties of the present disclosure are universal antibody binding moieties that bind to Fc regions. In some embodiments, binding of universal antibody binding moieties to Fc regions can happen at the same time as binding of Fc receptors, e.g., CD16a, to the same Fc regions (e.g., may at different locations/amino acid residues of the same Fc regions). In some embodiments, upon binding of universal antibody binding moieties, e.g., those in provided agents, compounds, methods, etc., an Fc region can still interact with Fc receptors and perform one or more or all of its immune activities, including recruitment of immune cells (e.g., effector cells such as NK cells), and/or triggering, generating, encouraging, and/or enhancing immune system activities toward target cells, tissues, objects and/or entities, for example, antibody-dependent cell-mediated cytotoxicity (ADCC) and/or ADCP.

Various universal antibody binding moieties can be utilized in accordance with the present disclosure. Certain antibody binding moieties and technologies for identifying and/or assessing universal antibody binding moieties and/or their utilization in ARMs are described in WO/2019/023501 and are incorporated herein by reference. Those skilled in the art appreciates that additional technologies in the art may be suitable for identifying and/or assessing universal antibody binding moieties suitable for ARMs in accordance with the present disclosure. In some embodiments, a universal antibody binding moiety comprises one or more amino acid residues, each independently natural or unnatural. In some embodiments, a universal antibody binding moiety has the structure of

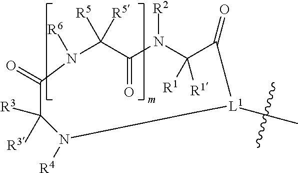

or a salt form thereof. In some embodiments, a universal antibody binding moiety has the structure of

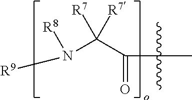

or a salt form thereof. In some embodiments, a universal antibody binding moiety is or comprises a peptide moiety, e.g., a moiety having the structure of R^{c}-(Xaa)z- or a salt form thereof, wherein each of R^{c}, z and Xaa is independently as described herein. In some embodiments, one or more Xaa are independently an unnatural amino acid residue. In some embodiments, side chains of two or more amino acid residues may be linked together to form bridges. For example, in some embodiments, side chains of two cysteine residues may form a disulfide bridge comprising —S—S— (which, as in many proteins, can be formed by two —SH groups). In some embodiments, a universal antibody binding moiety is or comprises a cyclic peptide moiety, e.g., a moiety having the structure of

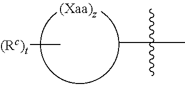

or a salt form thereof. In some embodiments, a universal antibody binding moiety is R^{c}-(Xaa)z- or

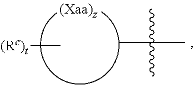

or a salt form thereof, and is or comprises a peptide unit. In some embodiments, -(Xaa)z- is or comprises a peptide unit. In some embodiments, a peptide unit comprises an amino acid residue (e.g., at physiological pH about 7.4, “positively charged amino acid residue”, Xaa^{P}), e.g., a residue of an amino acid of formula A-I that has a positively charged side chain. In some embodiments, a peptide unit comprises R. In some embodiments, at least one Xaa is R. In some embodiments, a peptide unit is or comprises APAR. In some embodiments, a peptide unit is or comprises RAPA. In some embodiments, a peptide unit comprises an amino acid residue, e.g., a residue of an amino acid of formula A-I, that has a side chain comprising an aromatic group (“aromatic amino acid residue”, Xaa^{A}). In some embodiments, a peptide unit comprises a positively charged amino acid residue and an aromatic amino acid residue. In some embodiments, a peptide unit comprises W. In some embodiments, a peptide unit comprises a positively charged amino acid residue and an aromatic amino acid residue. In some embodiments, a peptide unit is or comprises Xaa^{A}XaaXaa^{P}Xaa^{P}. In some embodiments, a peptide unit is or comprises Xaa^{P}Xaa^{P}XaaXaa^{A}. In some embodiments, a peptide unit is or comprises Xaa^{P}Xaa^{A}Xaa^{P}. In some embodiments, a peptide unit is or comprises two or more Xaa^{P}Xaa^{A}Xaa^{P}. In some embodiments, a peptide unit is or comprises Xaa^{P}Xaa^{A}Xaa^{P}XaaXaa^{P}Xaa^{A}Xaa^{P}. In some embodiments, a peptide unit is or comprises Xaa^{P}Xaa^{P}Xaa^{A}Xaa^{A}Xaa^{P}. In some embodiments, a peptide unit is or comprises Xaa^{P}Xaa^{P}Xaa^{P}Xaa^{A}. In some embodiments, a peptide unit is or comprises two or more Xaa^{A}Xaa^{A}Xaa^{P}. In some embodiments, a peptide residue comprises one or more proline residues. In some embodiments, a peptide unit is or comprises HWRGWA. In some embodiments, a peptide unit is or comprises WGRR. In some embodiments, a peptide unit is or comprises RRGW. In some embodiments, a peptide unit is or comprises NKFRGKYK. In some embodiments, a peptide unit is or comprises NRFRGKYK. In some embodiments, a peptide unit is or comprises NARKFYK. In some embodiments, a peptide unit is or comprises NARKFYKG. In some embodiments, a peptide unit is or comprises HWRGWV. In some embodiments, a peptide unit is or comprises KHFRNKD. In some embodiments, a peptide unit comprises a positively charged amino acid residue, an aromatic amino acid residue, and an amino acid residue, e.g., a residue of an amino acid of formula A-I, that has a negatively charged side chain (e.g., at physiological pH about 7.4, “negatively charged amino acid residue”, Xaa^{N}). In some embodiments, a peptide residue is RHRFNKD. In some embodiments, a peptide unit is TY. In some embodiments, a peptide unit is TYK. In some embodiments, a peptide unit is RTY. In some embodiments, a peptide unit is RTYK. In some embodiments, a peptide unit is or comprises a sequence selected from PAM. In some embodiments, a peptide unit is WHL. In some embodiments, a peptide unit is ELVW. In some embodiments, a peptide unit is or comprises a sequence selected from AWHLGELVW. In some embodiments, a peptide unit is or comprises a sequence selected from DCAWHLGELVWCT, which the two cysteine residues can form a disulfide bond as found in natural proteins. In some embodiments, a peptide unit is or comprises a sequence selected from Fc-III. In some embodiments, a peptide unit is or comprises a sequence selected from DpLpAWHLGELVW. In some embodiments, a peptide unit is or comprises a sequence selected from FcBP-1. In some embodiments, a peptide unit is or comprises a sequence selected from DpLpDCAWHLGELVWCT. In some embodiments, a peptide unit is or comprises a sequence selected from FcBP-2. In some embodiments, a peptide unit is or comprises a sequence selected from CDCAWHLGELVWCTC, wherein the first and the last cysteines, and the two cysteines in the middle of the sequence, can each independently form a disulfide bond as in natural proteins. In some embodiments, a peptide unit is or comprises a sequence selected from Fc-III-4c. In some embodiments, a peptide unit is or comprises a sequence selected from FcRM. In some embodiments, a peptide unit is or comprises a cyclic peptide unit. In some embodiments, a cyclic peptide unit comprises amide group formed by an amino group of a side chain and the C-terminus —COOH.

In some embodiments, -(Xaa)z- is or comprises [X¹]ₚ₁[X²]ₚ₂-X³X⁴X⁵X⁶X⁷X⁸X⁹X¹⁰X¹¹X¹²-[X¹³]ₚ₁₃-[X¹⁴]ₚ₁₄[X¹⁵]ₚ₁₅[X¹⁶]ₚ₁₆, wherein each of X¹, X², X³, X⁴, X⁵, X⁶, X⁷, X⁸, X⁹, X¹⁰, X¹¹, X¹², and X¹³ is independently an amino acid residue, e.g., of an amino acid of formula A-I, and each of p1, p2, p13, p14, p15 and p16 is independently 0, 1, 2, 3, 4, 5, 6, 7, 8, 9, or 10. In some embodiments, each of X¹, X², X³, X⁴, X⁵, X⁶, X⁷, X⁸, X⁹, X¹⁰, X¹¹, X¹², and X¹³ is independently an amino acid residue of an amino acid of formula A-I. In some embodiments, each of X¹, X², X³, X⁴, X⁵, X⁶, X⁷, X⁸, X⁹, X¹⁰, X¹¹, X¹², and X¹³ is independently a natural amino acid residue. In some embodiments, one or more of X¹, X², X³, X⁴, X⁵, X⁶, X⁷, X⁸, X⁹, X¹⁰, X¹¹, X¹², and X¹³ are independently an unnatural amino acid residue as described in the present disclosure.

In some embodiments, a peptide unit comprises a functional group in an amino acid residue that can react with a functional group of another amino acid residue. In some embodiments, a peptide unit comprises an amino acid residue with a side chain which comprises a functional group that can react with another functional group of the side chain of another amino acid residue to form a linkage (e.g., see moieties described in Table A-1, Table 1, etc.). In some embodiments, one functional group of one amino acid residue is connected to a functional group of another amino acid residue to form a linkage (or bridge). Linkages are bonded to backbone atoms of peptide units and comprise no backbone atoms. In some embodiments, a peptide unit comprises a linkage formed by two side chains of non-neighboring amino acid residues. In some embodiments, a linkage is bonded to two backbone atoms of two non-neighboring amino acid residues. In some embodiments, both backbone atoms bonded to a linkage are carbon atoms. In some embodiments, a linkage has the structure of L^{b}, wherein L^{b} is L^{a} as described in the present disclosure, wherein L^{a} is not a covalent bond. In some embodiments, L^{a} comprises -Cy-. In some embodiments, L^{a} comprises -Cy-, wherein -Cy- is optionally substituted heteroaryl. In some embodiments, -Cy- is

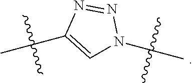 [SMILES: *c1cn(*)nn1]

In some embodiments, L^{a} is

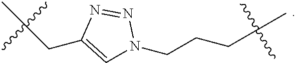 [SMILES: *CCCn1cc(C*)nn1]

In some embodiments, such an L^{a} can be formed by a —N₃ group of the side chain of one amino acid residue, and the —≡— of the side chain of another amino acid residue. In some embodiments, a linkage is formed through connection of two thiol groups, e.g., of two cysteine residues. In some embodiments, L^{a} comprises —S—S—. In some embodiments, L^{a} is —CH₂—S—S—CH₂—. In some embodiments, a linkage is formed through connection of an amino group (e.g., —NH₂ in the side chain of a lysine residue) and a carboxylic acid group (e.g., —COOH in the side chain of an aspartic acid or glutamic acid residue). In some embodiments, L^{a} comprises —C(O)—N(R′)—. In some embodiments, L^{a} comprise —C(O)—NH—. In some embodiments, L^{a} is —CH₂CONH—(CH₂)₃—. In some embodiments, L^{a} comprises —C(O)—N(R′)—, wherein R′ is R, and is taken together with an R group on the peptide backbone to form a ring (e.g., in A-34). In some embodiments, L^{a} is —(CH₂)₂—N(R′)—CO—(CH₂)₂—. In some embodiments, -Cy- is optionally substituted phenylene. In some embodiments, -Cy- is optionally substituted 1,2-phenylene. In some embodiments, L^{a} is

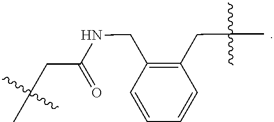 [SMILES: *CC(=O)NCc1ccccc1C*]

In some embodiments, L^{a} is

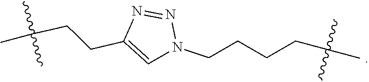 [SMILES: *CCCCn1cc(CCC*)nn1]

In some embodiments, L^{a} is optionally substituted bivalent C₂₋₂₀ bivalent aliphatic. In some embodiments, L^{a} is optionally substituted —(CH₂)₉—CH═CH—(CH₂)₉—. In some embodiments, L^{a} is —(CH₂)₃—CH═CH—(CH₂)₃—.

In some embodiments, two amino acid residues bonded to a linkage are separated by 1, 2, 3, 4, 5, 6, 7, 8, 9, 10, 11, 12, 13, 14, 15, or more than 15 amino acid residues between them (excluding the two amino acid residues bonded to the linkage). In some embodiments, the number is 1. In some embodiments, the number is 2. In some embodiments, the number is 3. In some embodiments, the number is 4. In some embodiments, the number is 5. In some embodiments, the number is 6. In some embodiments, the number is 7. In some embodiments, the number is 8. In some embodiments, the number is 9. In some embodiments, the number is 10. In some embodiments, the number is 11. In some embodiments, the number is 12. In some embodiments, the number is 13. In some embodiments, the number is 14. In some embodiments, the number is 15.

In some embodiments, each of p1, p2, p13, p14, p15 and p16 is 0. In some embodiments, -(Xaa)z- is or comprises —X³X⁴X⁵X⁶X⁷X⁸X⁹X¹⁰X¹¹X¹²—, wherein:

each of X³, X⁴, X⁵, X⁶, X⁷, X⁸, X⁹, X¹⁰, X¹¹, and X¹² is independently an amino acid residue;

X⁶ is Xaa^{A} or Xaa^{P};

X⁹ is Xaa^{N}; and

X¹² is Xaa^{A} or Xaa^{P}.

In some embodiments, each of X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, X⁵ is Xaa^{A} or Xaa^{P}. In some embodiments, X⁵ is Xaa^{A}. In some embodiments, X⁵ is Xaa^{P}. In some embodiments, X⁵ is an amino acid residue whose side chain comprises an optionally substituted saturated, partially saturated or aromatic ring. In some embodiments, X⁵ is

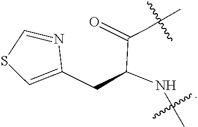 [SMILES: *CC(=O)[C@H](Cc1cscn1)N*]

In some embodiments, X⁵ is

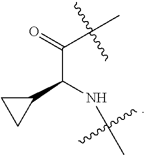 [SMILES: *N[C@H](C(*)=O)C1CC1]

In some embodiments, X⁶ is Xaa^{A}. In some embodiments, X⁶ is Xaa^{P}. In some embodiments, X⁶ is His. In some embodiments, X¹² is Xaa^{A}. In some embodiments, X¹² is Xaa^{P}. In some embodiments, X⁹ is Asp. In some embodiments, X⁹ is Glu. In some embodiments, X¹² is

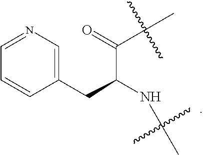 [SMILES: *N[C@@H](Cc1cccnc1)C(*)=O]

In some embodiments, X¹² is

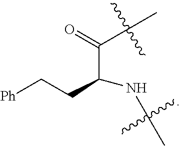 [SMILES: *N[C@@H](CCc1ccccc1)C(*)=O]

In some embodiments, each of X⁷, X¹⁰, and X¹¹ is independently an amino acid residue with a hydrophobic side chain (“hydrophobic amino acid residue”, Xaa^{H}). In some embodiments, X⁷ is Xaa^{H}. In some embodiments, X⁷ is

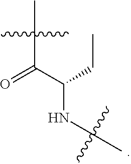 [SMILES: *N[C@@H](CC)C(*)=O]

In some embodiments, X⁷ is Val. In some embodiments, X¹⁰ is Xaa^{H}. In some embodiments, X¹⁰ is Met. In some embodiments, X¹⁰ is

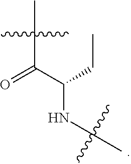 [SMILES: *N[C@@H](CC)C(*)=O]

In some embodiments, X¹¹ is Xaa^{H}. In some embodiments, X¹¹ is

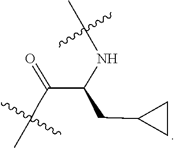 [SMILES: *CC(=O)[C@H](CC1CC1)N*]

In some embodiments, X⁸ is Gly. In some embodiments, X⁴ is Pro. In some embodiments, X³ is Lys. In some embodiments, the —COOH of X¹² forms an amide bond with the side chain amino group of Lys (X³), and the other amino group of the Lys (X³) is connected to a linker moiety and then a target binding moiety.

In some embodiments, -(Xaa)z- is or comprises —X³X⁴X⁵X⁶X⁷X⁸X⁹X¹⁰X¹¹X¹²—, wherein:

each of X³, X⁴, X⁵, X⁶, X⁷, X⁸, X⁹, X¹⁰, X¹¹, and X¹² is independently an amino acid residue;

at least two amino acid residues are connected through one or more linkages L^{b};

L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;

X⁶ is Xaa^{A} or Xaa^{P};

X⁹ is Xaa^{N}; and

X¹² is Xaa^{A} or Xaa^{P}.

In some embodiments, each of X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, X⁵ and X¹⁰ are connected by L^{b}. In some embodiments, there is one linkage L^{b}. In some embodiments, X⁶ is Xaa^{A}. In some embodiments, X⁶ is Xaa^{P}. In some embodiments, X⁶ is His. In some embodiments, X⁹ is Asp. In some embodiments, X⁹ is Glu. In some embodiments, X¹² is Xaa^{A}. In some embodiments, X¹² is

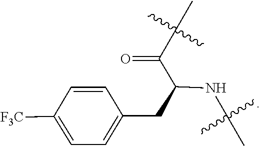 [SMILES: *N[C@@H](Cc1ccc(C(F)(F)F)cc1)C(*)=O]

In some embodiments, X¹² is

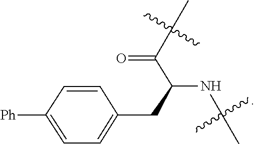 [SMILES: *N[C@@H](Cc1ccc(-c2ccccc2)cc1)C(*)=O]

In some embodiments, X¹² is

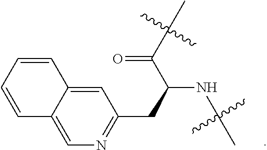 [SMILES: *N[C@@H](Cc1cc2ccccc2cn1)C(*)=O]

In some embodiments, each of X⁴, X⁷, and X¹¹ is independently Xaa^{H}. In some embodiments, X⁴ is Xaa^{H}. In some embodiments, X⁴ is Ala. In some embodiments, X⁷ is Xaa^{H}. In some embodiments, X⁷ is

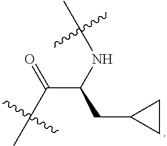 [SMILES: *N[C@@H](CC1CC1)C(*)=O]

In some embodiments, X¹¹ is Xaa^{H}. In some embodiments, X¹¹ is

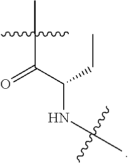 [SMILES: *N[C@@H](CC)C(*)=O]

In some embodiments, X⁸ is Gly. In some embodiments, X³ is Lys. In some embodiments, the —COOH of X¹² forms an amide bond with the side chain amino group of Lys (X³), and the other amino group of the Lys (X³) is connected to a linker moiety and then a target binding moiety. In some embodiments, L^{b} is

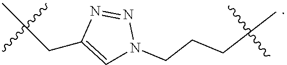 [SMILES: *CCCn1cc(C*)nn1]

In some embodiments, L^{b} is

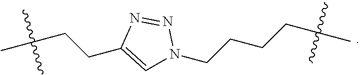 [SMILES: *CCCCn1cc(CC*)nn1]

In some embodiments, L^{b} connects two alpha-carbon atoms of two different amino acid residues. In some embodiments, both X⁵ and X¹⁰ are Cys, and the two —SH groups of their side chains form —S—S— (L^{b} is —CH₂—S—S—CH₂—).

In some embodiments, -(Xaa)z- is or comprises —X²X³X⁴X⁵X⁶X⁷X⁸X⁹X¹⁰X¹¹X¹²—, wherein:

each of X², X³, X⁴, X⁵, X⁶, X⁷, X⁸, X⁹, X¹⁰, X¹¹, and X¹² is independently an amino acid residue;

at least two amino acid residues are connected through one or more linkages L^{b};

L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;

X⁴ is Xaa^{A};

X⁵ is Xaa^{A} or Xaa^{P};

X⁸ is Xaa^{N}; and

X¹¹ is Xaa^{A}.

In some embodiments, each of X², X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, there is one linkage L^{b}. In some embodiments, X² and X¹¹ are connected by L^{b}. In some embodiments, L^{b} is —CH₂—S—S—CH₂—. In some embodiments, L^{b} is —CH₂—CH₂—S—CH₂—. In some embodiments, L^{b} is

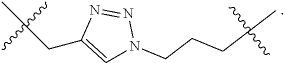 [SMILES: *CCCn1cc(C*)nn1]

In some embodiments, L^{b} is

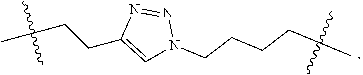 [SMILES: *CCCCn1cc(CC*)nn1]

In some embodiments, L^{b} is —CH₂CH₂CO—N(R′)—CH₂CH₂—. In some embodiments, R′ are taken together with an R group on the backbone atom that —N(R′)—CH₂CH₂— is bonded to to form a ring, e.g., as in A-34. In some embodiments, a formed ring is 3-, 4-, 5-, 6-, 7- or 8-membered. In some embodiments, a formed ring is monocyclic. In some embodiments, a formed ring is saturated. In some embodiments, L^{b} is

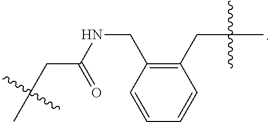 [SMILES: *CC(=O)NCc1ccccc1C*]

In some embodiments, L^{b} connects two alpha-carbon atoms of two different amino acid residues. In some embodiments, X⁴ is Xaa^{A}. In some embodiments, X⁴ is Tyr. In some embodiments, X⁵ is Xaa^{A}. In some embodiments, X⁵ is Xaa^{P}. In some embodiments, X⁵ is His. In some embodiments, X⁸ is Asp. In some embodiments, X⁸ is Glu. X¹¹ is Tyr. In some embodiments, both X² and X¹² are Cys, and the two —SH groups of their side chains form —S—S— (L^{b} is —CH₂—S—S—CH₂—). In some embodiments, each of X³, X⁶, X⁹, and X¹⁰ is independently Xaa^{H}. In some embodiments, X³ is Xaa^{H}. In some embodiments, X³ is Ala. In some embodiments, X⁶ is Xaa^{H}. In some embodiments, X⁶ is Leu. In some embodiments, X⁹ is Xaa^{H}. In some embodiments, X⁹ is Leu. In some embodiments, X⁹ is

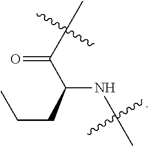 [SMILES: *N[C@@H](CCC)C(*)=O]

In some embodiments, X¹⁰ is Xaa^{H}. In some embodiments, X¹⁰ is Val. In some embodiments, X¹⁰ is

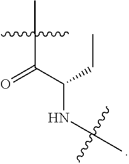 [SMILES: *N[C@@H](CC)C(*)=O]

In some embodiments, X⁷ is Gly. In some embodiments, p1 is 1. In some embodiments, X¹ is Asp. In some embodiments, p13 is 1. In some embodiments, p14, p15 and p16 are 0. In some embodiments, X¹³ is an amino acid residue comprising a polar uncharged side chain (e.g., at physiological pH, “polar uncharged amino acid residue”, Xaa^{L}). In some embodiments, X¹³ is Thr. In some embodiments, X¹³ is Val. In some embodiments, p13 is 0. In some embodiments, R^{c} is —NHCH₂CH(OH)CH₃. In some embodiments, R^{c} is (R)—NHCH₂CH(OH)CH₃. In some embodiments, R^{c} is (S)—NHCH₂CH(OH)CH₃.

In some embodiments, -(Xaa)z- is or comprises —X²X³X⁴X⁵X⁶X⁷X⁸X⁹X¹⁰X¹¹X¹²—, wherein:

each of X², X³, X⁴, X⁵, X⁶, X⁷, X⁸, X⁹, X¹⁰, X¹¹, and X¹² is independently an amino acid residue;

at least two amino acid residues are connected through one or more linkages L^{b};

L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;

X⁵ is Xaa^{A} or Xaa^{P};

X⁸ is Xaa^{N}; and

X¹¹ is Xaa^{A}.

In some embodiments, each of X², X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, there is one linkage L^{b}. In some embodiments, there are two or more linkages L^{b}. In some embodiments, there are two linkages L^{b}. In some embodiments, X² and X¹² are connected by L^{b}. In some embodiments, X⁴ and X⁹ are connected by L^{b}. In some embodiments, X⁴ and X¹⁰ are connected by L^{b}. In some embodiments, L^{b} is —CH₂—S—S—CH₂—. In some embodiments, L^{b} is

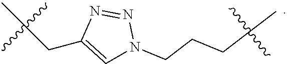 [SMILES: *CCCn1cc(C*)nn1]

In some embodiments, L^{b} is

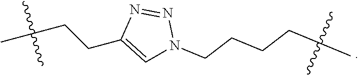 [SMILES: *CCCCn1cc(CC*)nn1]

In some embodiments, both X² and X¹² are Cys, and the two —SH groups of their side chains form —S—S— (L^{b} is —CH₂—S—S—CH₂—). In some embodiments, both X⁴ and X¹⁰ are Cys, and the two —SH groups of their side chains form —S—S— (L^{b} is —CH₂—S—S—CH₂—). In some embodiments, X⁴ and X⁹ are connected by L^{b}, wherein L^{b} is

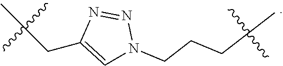 [SMILES: *CCCn1cc(C*)nn1]

In some embodiments, X⁴ and X⁹ are connected by L^{b}, wherein L^{b} is

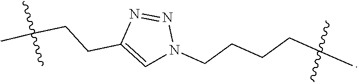 [SMILES: *CCCCn1cc(CC*)nn1]

In some embodiments, X⁵ is Xaa^{A}. In some embodiments, X⁵ is Xaa^{P}. In some embodiments, X⁵ is His. In some embodiments, X⁸ is Asp. In some embodiments, X⁸ is Glu. In some embodiments, X¹¹ is Tyr. In some embodiments, X¹¹ is

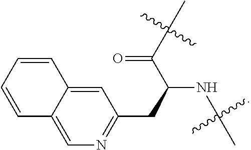 [SMILES: *N[C@@H](Cc1cc2ccccc2cn1)C(*)=O]

In some embodiments, X² and X¹² are connected by L^{b}, wherein L^{b} is —CH₂—S—CH₂CH₂—. In some embodiments, L^{b} connects two alpha-carbon atoms of two different amino acid residues. In some embodiments, each of X³, X⁶, and X⁹ is independently Xaa^{H}. In some embodiments, X³ is Xaa^{H}. In some embodiments, X³ is Ala. In some embodiments, X⁶ is Xaa^{H}. In some embodiments, X⁶ is Leu. In some embodiments, X⁶ is

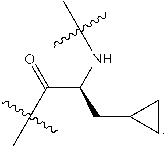 [SMILES: *N[C@@H](CC1CC1)C(*)=O]

In some embodiments, X⁹ is Xaa^{H}. In some embodiments, X⁹ is Leu. In some embodiments, X⁹ is

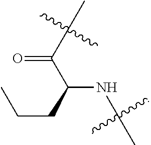 [SMILES: *N[C@@H](CCC)C(*)=O]

In some embodiments, X¹⁰ is Xaa^{H}. In some embodiments, X¹⁰ is Val. In some embodiments, X⁷ is Gly. In some embodiments, p1 is 1. In some embodiments, X¹ is Xaa^{N}. In some embodiments, X¹ is Asp. In some embodiments, X¹ is Glu. In some embodiments, p13 is 1. In some embodiments, p14, p15 and p16 are 0. In some embodiments, X¹³ is Xaa^{L}. In some embodiments, X¹³ is Thr. In some embodiments, X¹³ is Val.

In some embodiments, -(Xaa)z- is or comprises —X²X³X⁴X⁵X⁶X⁷X⁸X⁹X¹⁰, X¹¹X¹²X¹³X¹⁴X¹⁵X¹⁶—, wherein:

each of X², X³, X⁴, X⁵, X⁶, X⁷, X⁸, X⁹, X¹⁰, X¹¹, X¹², X¹³, X¹⁴, X¹⁵, and X¹⁶ is independently an amino acid residue;

at least two amino acid residues are connected through a linkage L^{b};

L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;

X³ is Xaa^{N};

X⁶ is Xaa^{A};

X⁷ is Xaa^{A} or Xaa^{P};

X⁹ is Xaa^{N}; and

X¹³ is Xaa^{A}.

In some embodiments, each of X², X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, there is one linkage L^{b}. In some embodiments, there are two or more linkages L^{b}. In some embodiments, there are two linkages L^{b}. In some embodiments, X² are connected to X¹⁶ by L^{b}. In some embodiments, X⁴ are connected to X¹⁴ by L^{b}. In some embodiments, both X² and X¹⁶ are Cys, and the two —SH groups of their side chains form —S—S— (L^{b} is —CH₂—S—S—CH₂—). In some embodiments, both X⁴ and X¹⁴ are Cys, and the two —SH groups of their side chains form —S—S— (L^{b} is —CH₂—S—S—CH₂—). In some embodiments, L^{b} connects two alpha-carbon atoms of two different amino acid residues. In some embodiments, X³ is Asp. In some embodiments, X³ is Glu. In some embodiments, X⁵ is Xaa^{H}. In some embodiments, X⁵ is Ala. In some embodiments, X⁶ is Xaa^{A}. In some embodiments, X⁶ is Tyr. In some embodiments, X⁷ is Xaa^{A}. In some embodiments, X⁷ is Xaa^{P}. In some embodiments, X⁷ is His. In some embodiments, X⁸ is Xaa^{H}. In some embodiments, X⁸ is Ala. In some embodiments, X⁹ is Gly. In some embodiments, X¹⁰ is Asp. In some embodiments, X¹⁰ is Glu. In some embodiments, X¹¹ is Xaa^{H}. In some embodiments, X¹¹ is Leu. In some embodiments, X¹² is Xaa^{H}. In some embodiments, X¹² is Val. In some embodiments, X¹³ is Xaa^{A}. In some embodiments, X¹³ is Tyr. In some embodiments, X¹⁵ is Xaa^{L}. In some embodiments, X¹⁵ is Thr. In some embodiments, X¹⁵ is Val. In some embodiments, p1 is 1. In some embodiments, In some embodiments, X¹ is Xaa^{N}. In some embodiments, X¹ is Asp. In some embodiments, X¹ is Glu.

As appreciated by those skilled in the art, an amino acid residue may be replaced by another amino acid residue having similar properties, e.g., one Xaa^{H} (e.g., Val, Leu, etc.) may be replaced with another Xaa^{H} (e.g., Leu, Ile, Ala, etc.), one Xaa^{A} may be replaced with another Xaa^{A}, one Xaa^{P} may be replaced with another Xaa^{P}, one Xaa^{N} may be replaced with another Xaa^{N}, one Xaa^{L} may be replaced with another Xaa^{L}, etc.

In some embodiments, an antibody binding moiety, e.g., a universal antibody binding moiety, is or comprises optionally substituted moiety of Table A-1. In some embodiments, an antibody binding moiety, e.g., a universal antibody binding moiety, is selected from able A-1.

TABLE A-1
Exemplary antibody binding moieties.

| | |
|---|---|
| 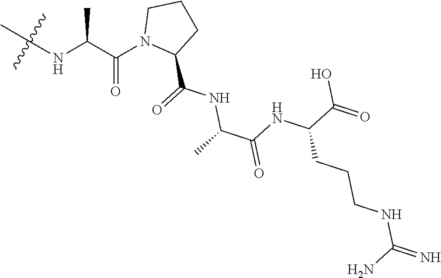 [SMILES: *N[C@@H](C)C(=O)N1CCC[C@H]1C(=O)N[C@@H](C)C(=O)N[C@@H](CCCNC(=N)N)C(=O)O] | A-1 |
| 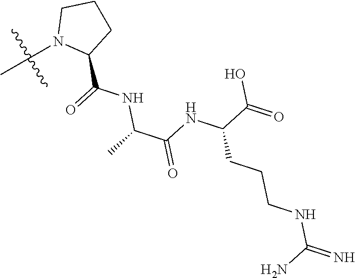 [SMILES: *N1CCC[C@H]1C(=O)N[C@@H](C)C(=O)N[C@@H](CCCNC(=N)N)C(=O)O] | A-2 |
| 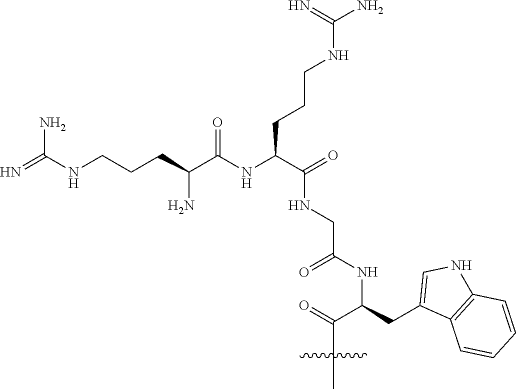 [SMILES: *C(=O)[C@H](Cc1c[nH]c2ccccc12)NC(=O)CNC(=O)[C@H](CCCNC(=N)N)NC(=O)[C@@H](N)CCCNC(=N)N] | A-3 |
| 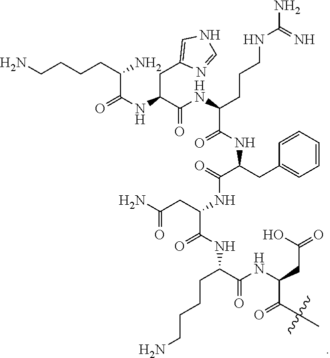 [SMILES: *C(=O)[C@H](CC(=O)O)NC(=O)[C@H](CCCCN)NC(=O)[C@H](CC(N)=O)NC(=O)[C@H](Cc1ccccc1)NC(=O)[C@H](CCCNC(=N)N)NC(=O)[C@H](Cc1c[nH]cn1)NC(=O)[C@@H](N)CCCCN] | A-4 |
| 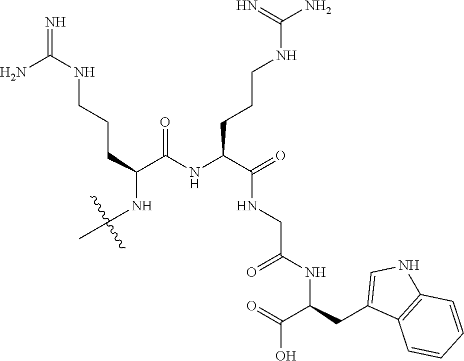 [SMILES: *N[C@@H](CCCNC(=N)N)C(=O)N[C@@H](CCCNC(=N)N)C(=O)NCC(=O)N[C@@H](Cc1c[nH]c2ccccc12)C(=O)O] | A-5 |
| 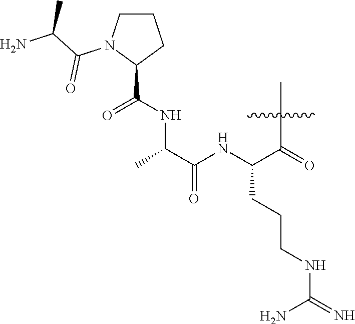 [SMILES: *C(=O)[C@H](CCCNC(=N)N)NC(=O)[C@H](C)NC(=O)[C@@H]1CCCN1C(=O)[C@H](C)N] | A-6 |
| 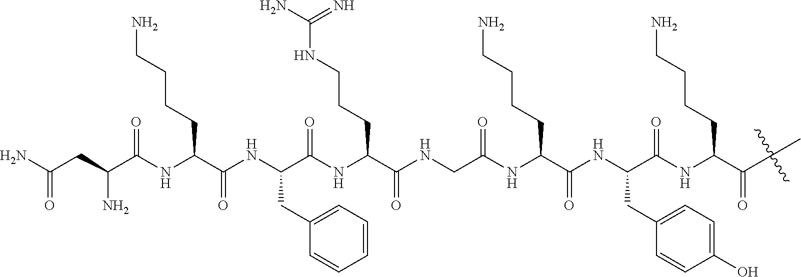 [SMILES: *C(=O)[C@H](CCCCN)NC(=O)[C@H](Cc1ccc(O)cc1)NC(=O)[C@H](CCCCN)NC(=O)CNC(=O)[C@H](CCCNC(=N)N)NC(=O)[C@H](Cc1ccccc1)NC(=O)[C@H](CCCCN)NC(=O)[C@@H](N)CC(N)=O] | A-7 |
| 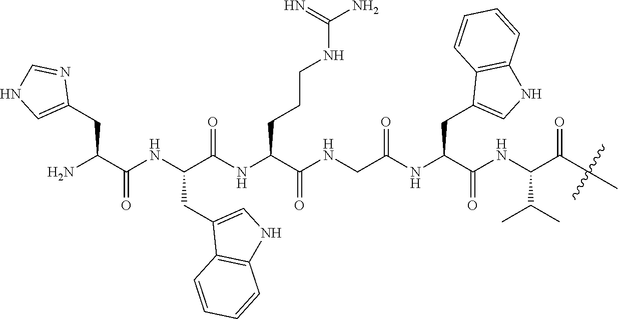 [SMILES: *C(=O)[C@@H](NC(=O)[C@H](Cc1c[nH]c2ccccc12)NC(=O)CNC(=O)[C@H](CCCNC(=N)N)NC(=O)[C@H](Cc1c[nH]c2ccccc12)NC(=O)[C@@H](N)Cc1c[nH]cn1)C(C)C] | A-8 |
| 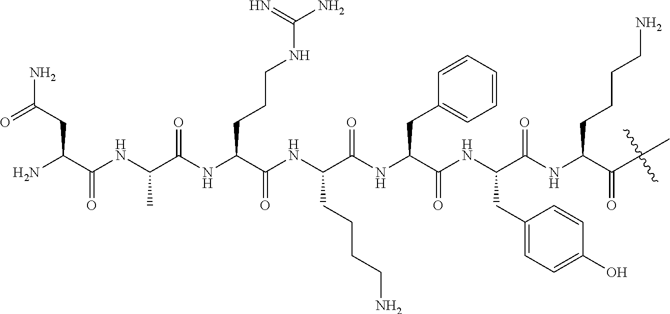 [SMILES: *C(=O)[C@H](CCCCN)NC(=O)[C@H](Cc1ccc(O)cc1)NC(=O)[C@H](Cc1ccccc1)NC(=O)[C@H](CCCCN)NC(=O)[C@H](CCCNC(=N)N)NC(=O)[C@H](C)NC(=O)[C@@H](N)CC(N)=O] | A-9 |
| 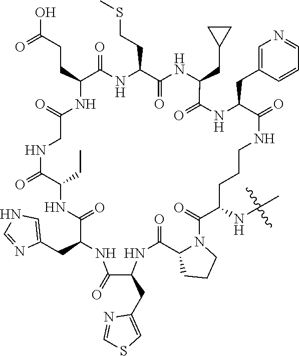 [SMILES: *N[C@H]1CCCNC(=O)[C@H](Cc2cccnc2)NC(=O)[C@H](CC2CC2)NC(=O)[C@H](CCSC)NC(=O)[C@H](CCC(=O)O)NC(=O)CNC(=O)[C@H](CC)NC(=O)[C@H](Cc2c[nH]cn2)NC(=O)[C@H](Cc2cscn2)NC(=O)[C@H]2CCCN2C1=O] | A-10 |
| 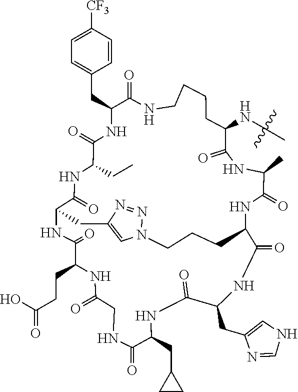 [SMILES: *N[C@@H]1CCCCNC(=O)[C@H](Cc2ccc(C(F)(F)F)cc2)NC(=O)[C@H](CC)NC(=O)[C@H]2Cc3cn(nn3)CCC[C@@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-11 |
| 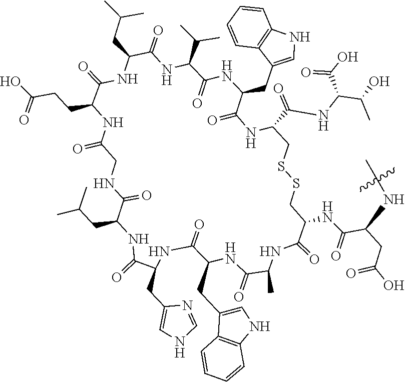 [SMILES: *N[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-12 |
| 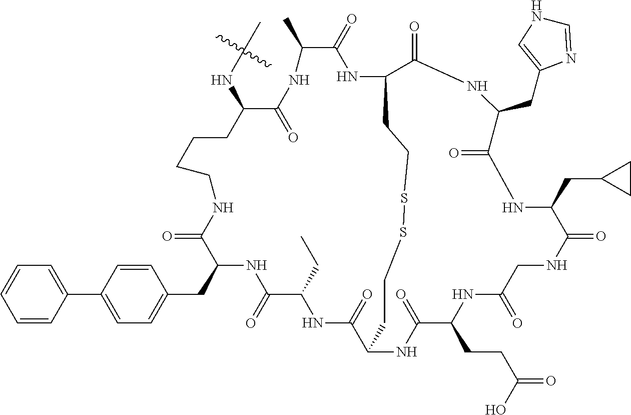 [SMILES: *N[C@@H]1CCCCNC(=O)[C@H](Cc2ccc(-c3ccccc3)cc2)NC(=O)[C@H](CC)NC(=O)[C@H]2CCSSCC[C@@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-13 |
| 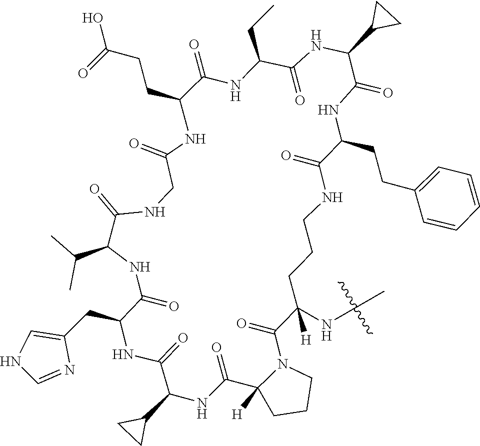 [SMILES: *N[C@H]1CCCNC(=O)[C@H](CCc2ccccc2)NC(=O)[C@H](C2CC2)NC(=O)[C@H](CC)NC(=O)[C@H](CCC(=O)O)NC(=O)CNC(=O)[C@H](C(C)C)NC(=O)[C@H](Cc2c[nH]cn2)NC(=O)[C@H](C2CC2)NC(=O)[C@H]2CCCN2C1=O] | A-14 |
| 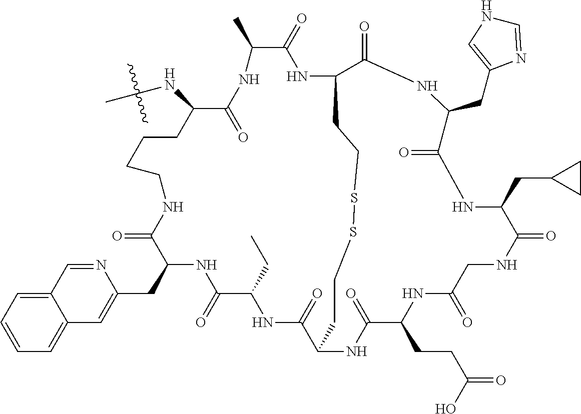 [SMILES: *N[C@@H]1CCCCNC(=O)[C@H](Cc2cc3ccccc3cn2)NC(=O)[C@H](CC)NC(=O)[C@H]2CCSSCC[C@@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-15 |
| 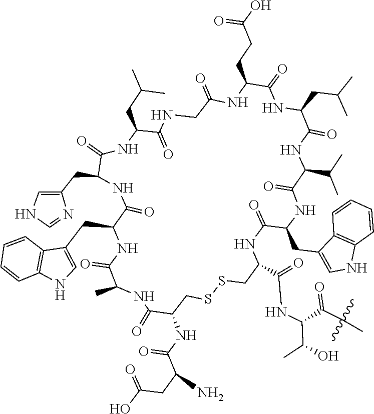 [SMILES: *C(=O)[C@@H](NC(=O)[C@@H]1CSSC[C@H](NC(=O)[C@@H](N)CC(=O)O)C(=O)N[C@@H](C)C(=O)N[C@@H](Cc2c[nH]c3ccccc23)C(=O)N[C@@H](Cc2c[nH]cn2)C(=O)N[C@@H](CC(C)C)C(=O)NCC(=O)N[C@@H](CCC(=O)O)C(=O)N[C@@H](CC(C)C)C(=O)N[C@@H](C(C)C)C(=O)N[C@@H](Cc2c[nH]c3ccccc23)C(=O)N1)[C@@H](C)O] | A-16 |
| 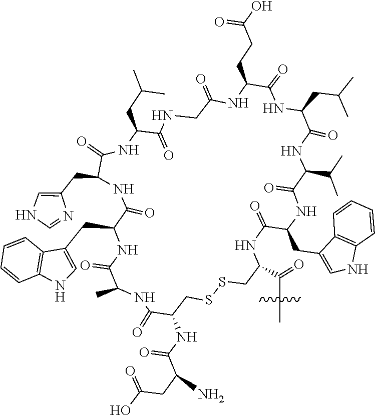 [SMILES: *C(=O)[C@@H]1CSSC[C@H](NC(=O)[C@@H](N)CC(=O)O)C(=O)N[C@@H](C)C(=O)N[C@@H](Cc2c[nH]c3ccccc23)C(=O)N[C@@H](Cc2c[nH]cn2)C(=O)N[C@@H](CC(C)C)C(=O)NCC(=O)N[C@@H](CCC(=O)O)C(=O)N[C@@H](CC(C)C)C(=O)N[C@@H](C(C)C)C(=O)N[C@@H](Cc2c[nH]c3ccccc23)C(=O)N1] | A-17 |
| 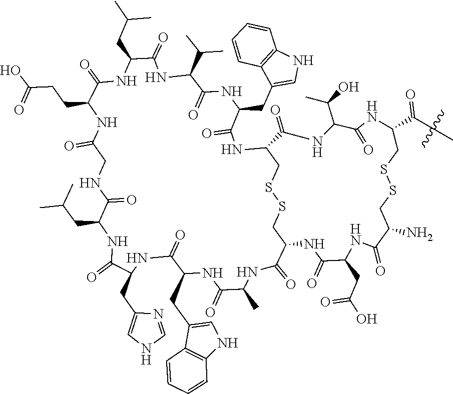 [SMILES: *C(=O)[C@@H]1CSSC[C@H](N)C(=O)N[C@@H](CC(=O)O)C(=O)N[C@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc3c[nH]cn3)NC(=O)[C@H](Cc3c[nH]c4ccccc34)NC(=O)[C@H](C)NC2=O)C(=O)NC([C@@H](C)O)C(=O)N1] | A-18 |
| 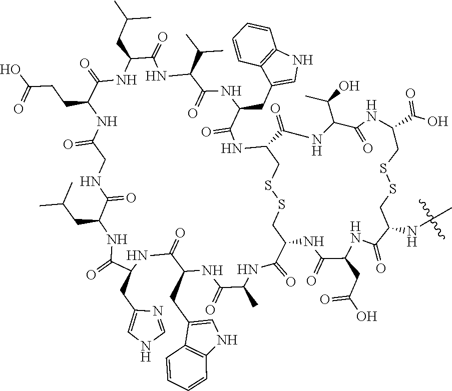 [SMILES: *N[C@H]1CSSC[C@@H](C(=O)O)NC(=O)C([C@@H](C)O)NC(=O)[C@@H]2CSSC[C@H](NC(=O)[C@H](CC(=O)O)NC1=O)C(=O)N[C@@H](C)C(=O)N[C@@H](Cc1c[nH]c3ccccc13)C(=O)N[C@@H](Cc1c[nH]cn1)C(=O)N[C@@H](CC(C)C)C(=O)NCC(=O)N[C@@H](CCC(=O)O)C(=O)N[C@@H](CC(C)C)C(=O)N[C@@H](C(C)C)C(=O)N[C@@H](Cc1c[nH]c3ccccc13)C(=O)N2] | A-19 |
| 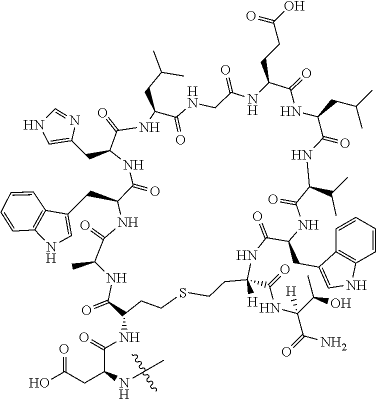 | A-20 |
| 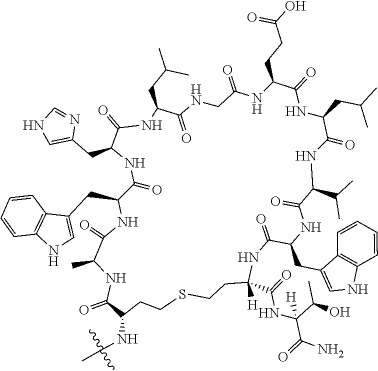 | A-21 |
| 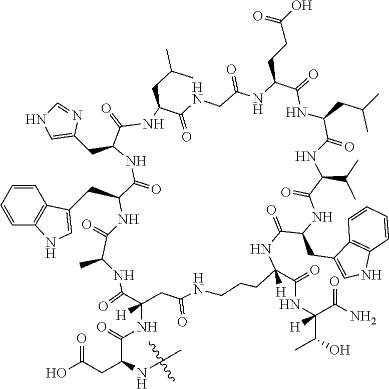 [SMILES: *N[C@@H](CC(=O)O)C(=O)N[C@H]1CC(=O)NCCC[C@@H](C(=O)N[C@H](C(N)=O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-22 |
| 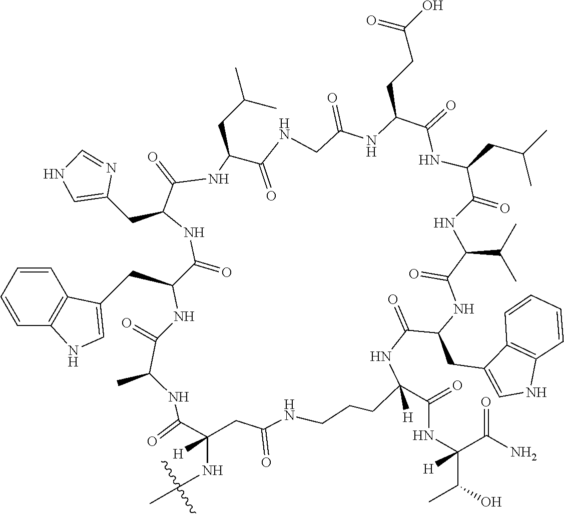 [SMILES: *N[C@H]1CC(=O)NCCC[C@@H](C(=O)N[C@H](C(N)=O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-23 |
| 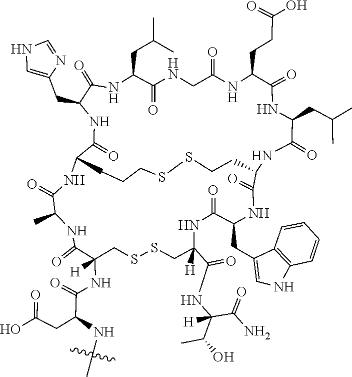 [SMILES: *N[C@@H](CC(=O)O)C(=O)N[C@H]1CSSC[C@@H](C(=O)N[C@H](C(N)=O)[C@@H](C)O)NC(=O)[C@H](Cc2c[nH]c3ccccc23)NC(=O)[C@@H]2CCSSCCC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CC(C)C)C(=O)N2] | A-24 |
| 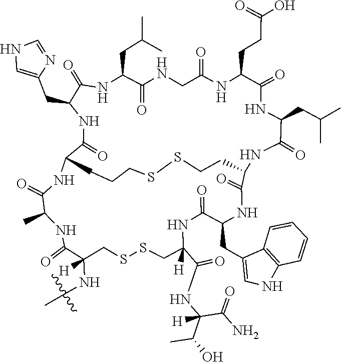 [SMILES: *N[C@H]1CSSC[C@@H](C(=O)N[C@H](C(N)=O)[C@@H](C)O)NC(=O)[C@H](Cc2c[nH]c3ccccc23)NC(=O)[C@@H]2CCSSCCC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CC(C)C)C(=O)N2] | A-25 |
| 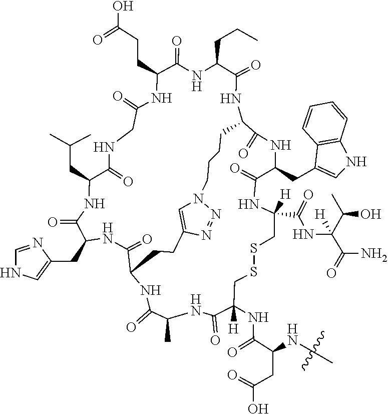 [SMILES: *N[C@@H](CC(=O)O)C(=O)N[C@H]1CSSC[C@@H](C(=O)N[C@H](C(N)=O)[C@@H](C)O)NC(=O)[C@H](Cc2c[nH]c3ccccc23)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CCC)C(=O)N2] | A-26 |
| 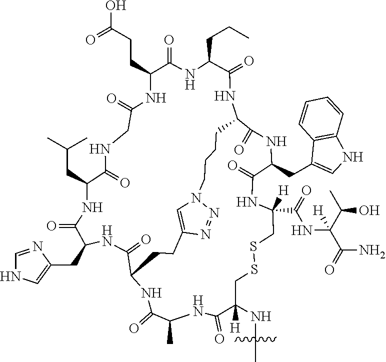 [SMILES: *N[C@H]1CSSC[C@@H](C(=O)N[C@H](C(N)=O)[C@@H](C)O)NC(=O)[C@H](Cc2c[nH]c3ccccc23)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CCC)C(=O)N2] | A-27 |
| 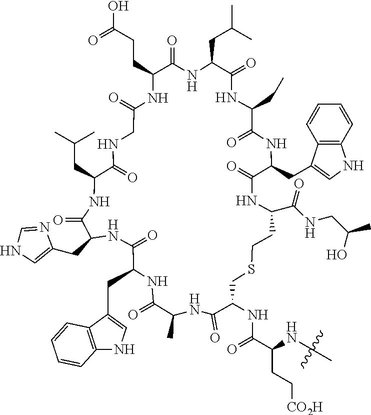 [SMILES: *N[C@@H](CCC(=O)O)C(=O)N[C@H]1CSCC[C@@H](C(=O)NC[C@@H](C)O)NC(=O)[C@H](Cc2c[nH]c3ccccc23)NC(=O)[C@H](CC)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-28 |
| 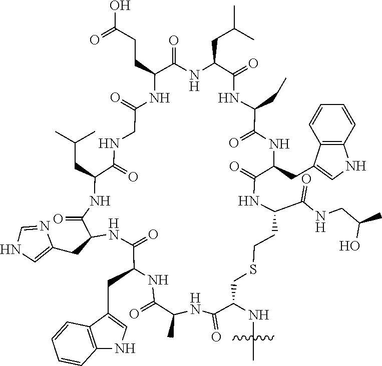 [SMILES: *N[C@H]1CSCC[C@@H](C(=O)NC[C@@H](C)O)NC(=O)[C@H](Cc2c[nH]c3ccccc23)NC(=O)[C@H](CC)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-29 |
| 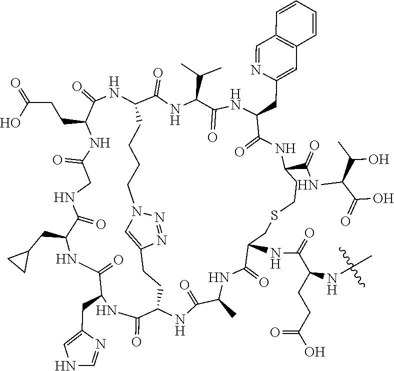 [SMILES: *N[C@@H](CCC(=O)O)C(=O)N[C@H]1CSCC[C@@H](C(=O)N[C@H](C(=O)O)C(C)O)NC(=O)[C@H](Cc2cc3ccccc3cn2)NC(=O)[C@H](C(C)C)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-30 |
| 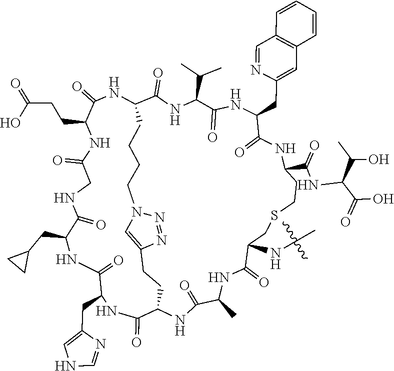 [SMILES: *N[C@H]1CSCC[C@@H](C(=O)N[C@H](C(=O)O)C(C)O)NC(=O)[C@H](Cc2cc3ccccc3cn2)NC(=O)[C@H](C(C)C)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-31 |
| 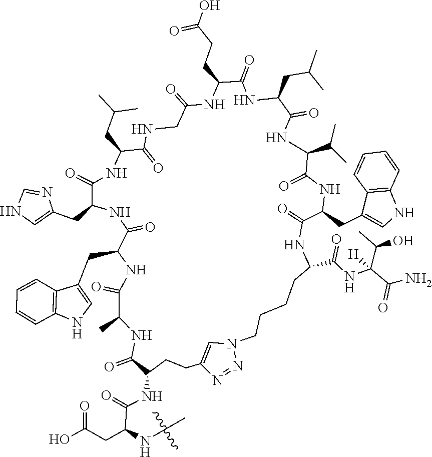 | A-32 |
| 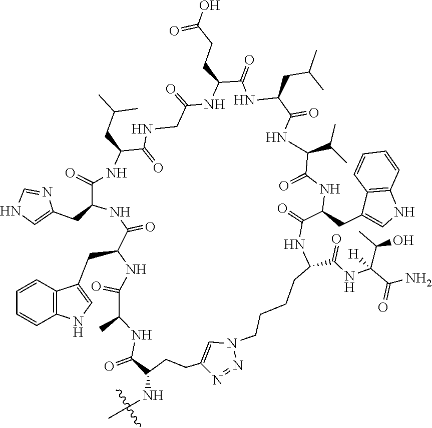 | A-33 |
| 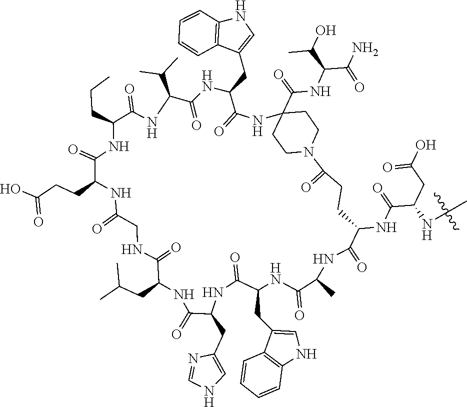 [SMILES: *N[C@@H](CC(=O)O)C(=O)N[C@H]1CCC(=O)N2CCC(C(=O)N[C@H](C(N)=O)C(C)O)(CC2)NC(=O)[C@H](Cc2c[nH]c3ccccc23)NC(=O)[C@H](C(C)C)NC(=O)[C@H](CCC)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-34 |
| 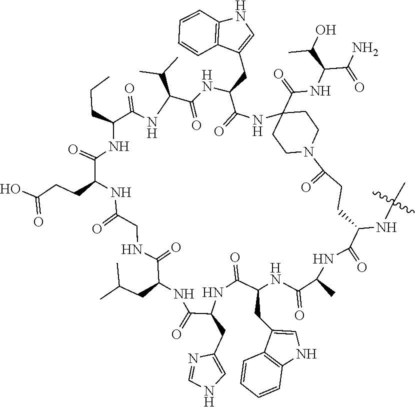 [SMILES: *N[C@H]1CCC(=O)N2CCC(C(=O)N[C@H](C(N)=O)C(C)O)(CC2)NC(=O)[C@H](Cc2c[nH]c3ccccc23)NC(=O)[C@H](C(C)C)NC(=O)[C@H](CCC)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-35 |
| 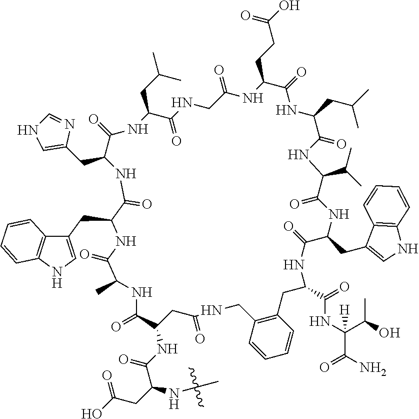 | A-36 |
| 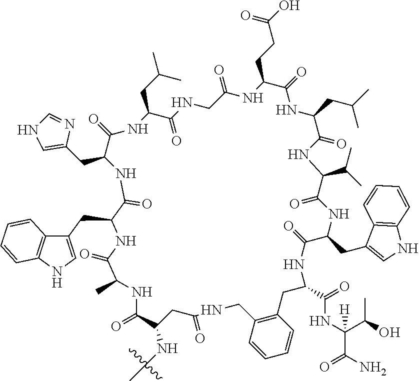 | A-37 |
| 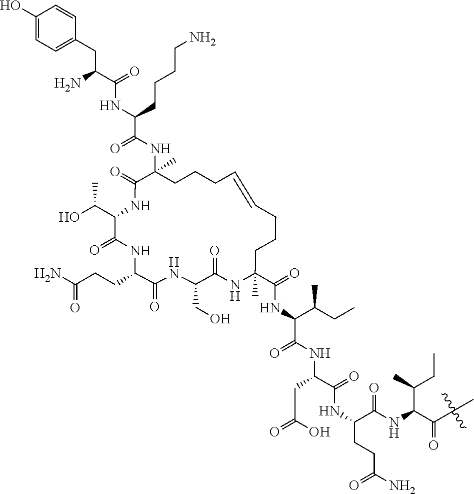 [SMILES: *C(=O)[C@@H](NC(=O)[C@H](CCC(N)=O)NC(=O)[C@H](CC(=O)O)NC(=O)[C@@H](NC(=O)[C@]1(C)CCC/C=C/CCC[C@](C)(NC(=O)[C@H](CCCCN)NC(=O)[C@@H](N)Cc2ccc(O)cc2)C(=O)N[C@@H]([C@@H](C)O)C(=O)N[C@@H](CCC(N)=O)C(=O)N[C@@H](CO)C(=O)N1)[C@@H](C)CC)[C@@H](C)CC] | A-38 |
| 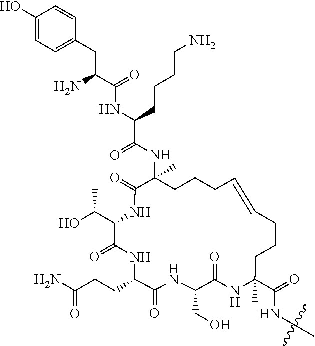 [SMILES: *NC(=O)[C@]1(C)CCC/C=C/CCC[C@](C)(NC(=O)[C@H](CCCCN)NC(=O)[C@@H](N)Cc2ccc(O)cc2)C(=O)N[C@@H]([C@@H](C)O)C(=O)N[C@@H](CCC(N)=O)C(=O)N[C@@H](CO)C(=O)N1] | A-39 |
| 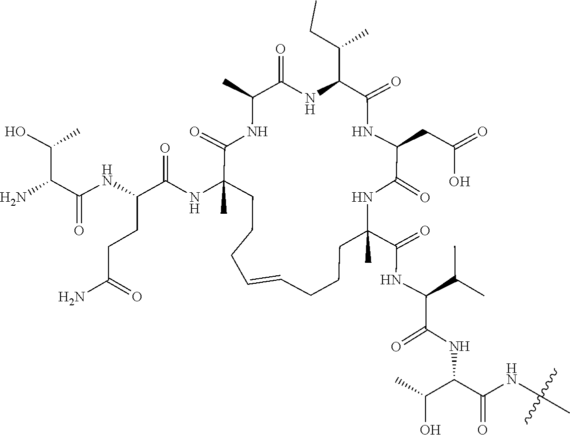 [SMILES: *NC(=O)[C@@H](NC(=O)[C@@H](NC(=O)[C@]1(C)CCC/C=C/CCC[C@](C)(NC(=O)[C@H](CCC(N)=O)NC(=O)[C@H](N)[C@@H](C)O)C(=O)N[C@@H](C)C(=O)N[C@@H]([C@@H](C)CC)C(=O)N[C@@H](CC(=O)O)C(=O)N1)C(C)C)[C@@H](C)O] | A-40 |
| 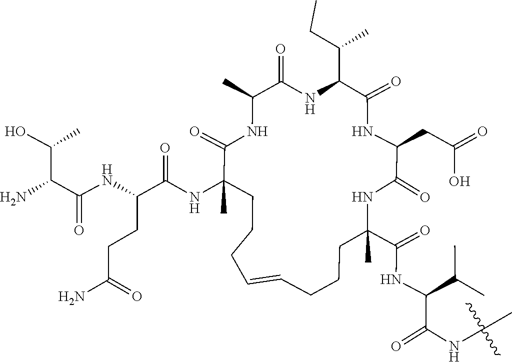 [SMILES: *NC(=O)[C@@H](NC(=O)[C@]1(C)CCC/C=C/CCC[C@](C)(NC(=O)[C@H](CCC(N)=O)NC(=O)[C@H](N)[C@@H](C)O)C(=O)N[C@@H](C)C(=O)N[C@@H]([C@@H](C)CC)C(=O)N[C@@H](CC(=O)O)C(=O)N1)C(C)C] | A-41 |
| 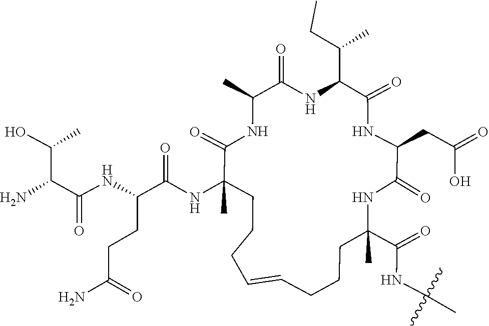 [SMILES: *NC(=O)[C@]1(C)CCC/C=C/CCC[C@](C)(NC(=O)[C@H](CCC(N)=O)NC(=O)[C@H](N)[C@@H](C)O)C(=O)N[C@@H](C)C(=O)N[C@@H]([C@@H](C)CC)C(=O)N[C@@H](CC(=O)O)C(=O)N1] | A-42 |
| 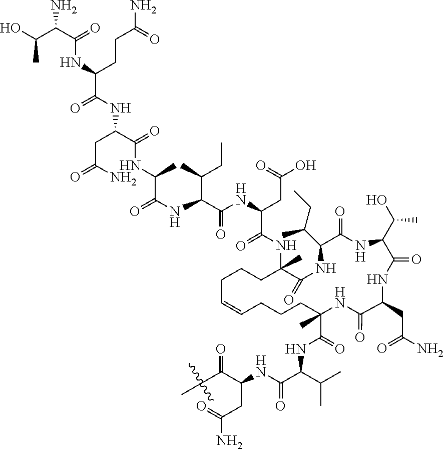 [SMILES: *C(=O)[C@H](CC(N)=O)NC(=O)[C@@H](NC(=O)[C@]1(C)CCC/C=C\CCC[C@](C)(NC(=O)[C@H](CC(=O)O)NC(=O)[C@@H](NC(=O)[C@H](C)NC(=O)[C@H](CC(N)=O)NC(=O)[C@H](CCC(N)=O)NC(=O)[C@@H](N)[C@@H](C)O)[C@@H](C)CC)C(=O)N[C@@H]([C@@H](C)CC)C(=O)N[C@@H]([C@@H](C)O)C(=O)N[C@@H](CC(N)=O)C(=O)N1)C(C)C] | A-43 |
| 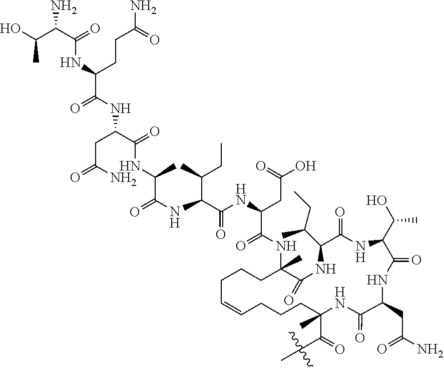 [SMILES: *C(=O)[C@]1(C)CCC/C=C\CCC[C@](C)(NC(=O)[C@H](CC(=O)O)NC(=O)[C@@H](NC(=O)[C@H](C)NC(=O)[C@H](CC(N)=O)NC(=O)[C@H](CCC(N)=O)NC(=O)[C@@H](N)[C@@H](C)O)[C@@H](C)CC)C(=O)N[C@@H]([C@@H](C)CC)C(=O)N[C@@H]([C@@H](C)O)C(=O)N[C@@H](CC(N)=O)C(=O)N1] | A-44 |
| 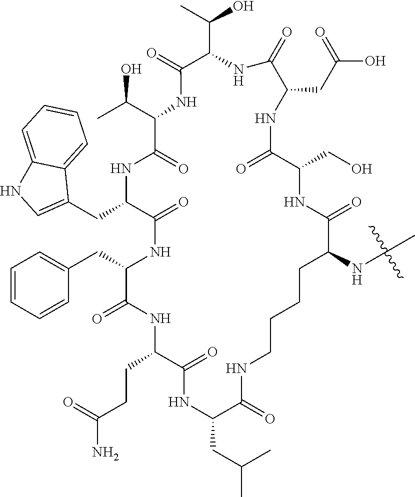 [SMILES: *N[C@H]1CCCCNC(=O)[C@H](CC(C)C)NC(=O)[C@H](CCC(N)=O)NC(=O)[C@H](Cc2ccccc2)NC(=O)[C@H](Cc2c[nH]c3ccccc23)NC(=O)[C@H]([C@@H](C)O)NC(=O)[C@H]([C@@H](C)O)NC(=O)[C@H](CC(=O)O)NC(=O)[C@H](CO)NC1=O] | A-45 |
| 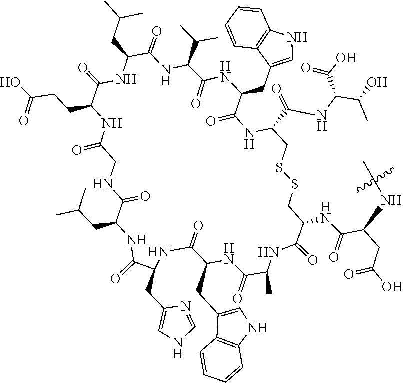 [SMILES: *N[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-46 |
| 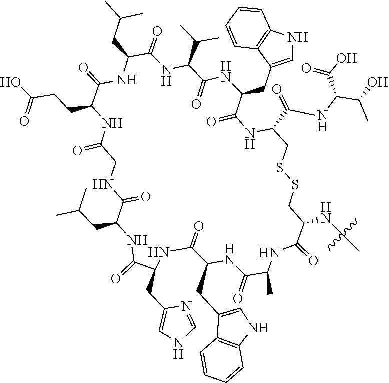 [SMILES: *N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-47 |
| 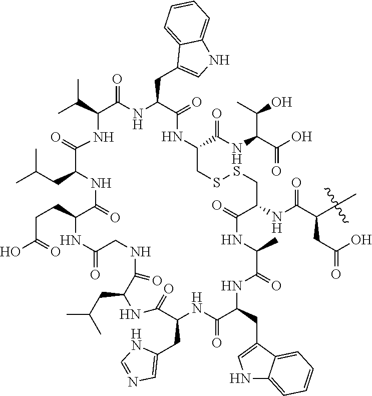 [SMILES: *[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2cnc[nH]2)NC(=O)[C@H](Cc2c[nH]c3ccccc23)NC(=O)[C@H](C)NC1=O] | A-48 |
| or | |
| 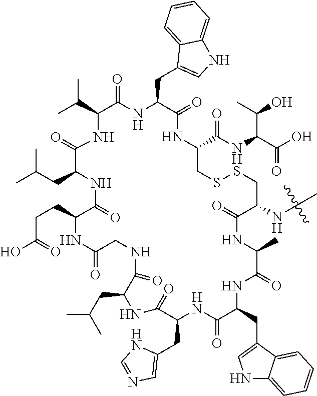 [SMILES: *N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2cnc[nH]2)NC(=O)[C@H](Cc2c[nH]c3ccccc23)NC(=O)[C@H](C)NC1=O] | A-49 |

In some embodiments, a universal antibody binding moiety is or comprises optionally substituted A-1. In some embodiments, a universal antibody binding moiety is or comprises optionally substituted A-2. In some embodiments, a universal antibody binding moiety is or comprises optionally substituted A-3. In some embodiments, a universal antibody binding moiety is or comprises optionally substituted A-4. In some embodiments, a universal antibody binding moiety is or comprises optionally substituted A-5. In some embodiments, a universal antibody binding moiety is or comprises optionally substituted A-6. In some embodiments, a universal antibody binding moiety is or comprises optionally substituted A-7. In some embodiments, a universal antibody binding moiety is or comprises optionally substituted A-8. In some embodiments, a universal antibody binding moiety is or comprises optionally substituted A-9. In some embodiments, a universal antibody binding moiety is or comprises optionally substituted A-10. In some embodiments, a universal antibody binding moiety is or comprises optionally substituted A-11. In some embodiments, a universal antibody binding moiety is or comprises optionally substituted A-12. In some embodiments, a universal antibody binding moiety is or comprises optionally substituted A-13. In some embodiments, a universal antibody binding moiety is or comprises optionally substituted A-14. In some embodiments, a universal antibody binding moiety is or comprises optionally substituted A-15. In some embodiments, a universal antibody binding moiety is or comprises optionally substituted A-16. In some embodiments, a universal antibody binding moiety is or comprises optionally substituted A-17. In some embodiments, a universal antibody binding moiety is or comprises optionally substituted A-18. In some embodiments, a universal antibody binding moiety is or comprises optionally substituted A-19. In some embodiments, a universal antibody binding moiety is or comprises optionally substituted A-20. In some embodiments, a universal antibody binding moiety is or comprises optionally substituted A-21. In some embodiments, a universal antibody binding moiety is or comprises optionally substituted A-22. In some embodiments, a universal antibody binding moiety is or comprises optionally substituted A-23. In some embodiments, a universal antibody binding moiety is or comprises optionally substituted A-24. In some embodiments, a universal antibody binding moiety is or comprises optionally substituted A-25. In some embodiments, a universal antibody binding moiety is or comprises optionally substituted A-26. In some embodiments, a universal antibody binding moiety is or comprises optionally substituted A-27. In some embodiments, a universal antibody binding moiety is or comprises optionally substituted A-28. In some embodiments, a universal antibody binding moiety is or comprises optionally substituted A-29. In some embodiments, a universal antibody binding moiety is or comprises optionally substituted A-30. In some embodiments, a universal antibody binding moiety is or comprises optionally substituted A-31. In some embodiments, a universal antibody binding moiety is or comprises optionally substituted A-32. In some embodiments, a universal antibody binding moiety is or comprises optionally substituted A-33. In some embodiments, a universal antibody binding moiety is or comprises optionally substituted A-34. In some embodiments, a universal antibody binding moiety is or comprises optionally substituted A-35. In some embodiments, a universal antibody binding moiety is or comprises optionally substituted A-36. In some embodiments, a universal antibody binding moiety is or comprises optionally substituted A-37. In some embodiments, a universal antibody binding moiety is or comprises optionally substituted A-38. In some embodiments, a universal antibody binding moiety is or comprises optionally substituted A-39. In some embodiments, a universal antibody binding moiety is or comprises optionally substituted A-40. In some embodiments, a universal antibody binding moiety is or comprises optionally substituted A-41. In some embodiments, a universal antibody binding moiety is or comprises optionally substituted A-42. In some embodiments, a universal antibody binding moiety is or comprises optionally substituted A-43. In some embodiments, a universal antibody binding moiety is or comprises optionally substituted A-44. In some embodiments, a universal antibody binding moiety is or comprises optionally substituted A-45. In some embodiments, a universal antibody binding moiety is or comprises optionally substituted A-46. In some embodiments, a universal antibody binding moiety is or comprises optionally substituted A-47. In some embodiments, a universal antibody binding moiety is or comprises optionally substituted A-48. In some embodiments, a universal antibody binding moiety is or comprises optionally substituted A-49.

In some embodiments, a universal antibody binding moiety is A-1. In some embodiments, a universal antibody binding moiety is A-2. In some embodiments, a universal antibody binding moiety is A-3. In some embodiments, a universal antibody binding moiety is A-4. In some embodiments, a universal antibody binding moiety is A-5. In some embodiments, a universal antibody binding moiety is A-6. In some embodiments, a universal antibody binding moiety is A-7. In some embodiments, a universal antibody binding moiety is A-8. In some embodiments, a universal antibody binding moiety is A-9. In some embodiments, a universal antibody binding moiety is A-10. In some embodiments, a universal antibody binding moiety is A-11. In some embodiments, a universal antibody binding moiety is A-12. In some embodiments, a universal antibody binding moiety is A-13. In some embodiments, a universal antibody binding moiety is A-14. In some embodiments, a universal antibody binding moiety is A-15. In some embodiments, a universal antibody binding moiety is A-16. In some embodiments, a universal antibody binding moiety is A-17. In some embodiments, a universal antibody binding moiety is A-18. In some embodiments, a universal antibody binding moiety is A-19. In some embodiments, a universal antibody binding moiety is A-20. In some embodiments, a universal antibody binding moiety is A-21. In some embodiments, a universal antibody binding moiety is A-22. In some embodiments, a universal antibody binding moiety is A-23. In some embodiments, a universal antibody binding moiety is A-24. In some embodiments, a universal antibody binding moiety is A-25. In some embodiments, a universal antibody binding moiety is A-26. In some embodiments, a universal antibody binding moiety is A-27. In some embodiments, a universal antibody binding moiety is A-28. In some embodiments, a universal antibody binding moiety is A-29. In some embodiments, a universal antibody binding moiety is A-30. In some embodiments, a universal antibody binding moiety is A-31. In some embodiments, a universal antibody binding moiety is A-32. In some embodiments, a universal antibody binding moiety is A-33. In some embodiments, a universal antibody binding moiety is A-34. In some embodiments, a universal antibody binding moiety is A-35. In some embodiments, a universal antibody binding moiety is A-36. In some embodiments, a universal antibody binding moiety is A-37. In some embodiments, a universal antibody binding moiety is A-38. In some embodiments, a universal antibody binding moiety is A-39. In some embodiments, a universal antibody binding moiety is A-40. In some embodiments, a universal antibody binding moiety is A-41. In some embodiments, a universal antibody binding moiety is A-42. In some embodiments, a universal antibody binding moiety is A-43. In some embodiments, a universal antibody binding moiety is A-44. In some embodiments, a universal antibody binding moiety is A-45. In some embodiments, a universal antibody binding moiety is A-46. In some embodiments, a universal antibody binding moiety is A-47. In some embodiments, a universal antibody binding moiety is A-48. In some embodiments, a universal antibody binding moiety is A-49.

In some embodiments, a antibody binding moiety is or comprises

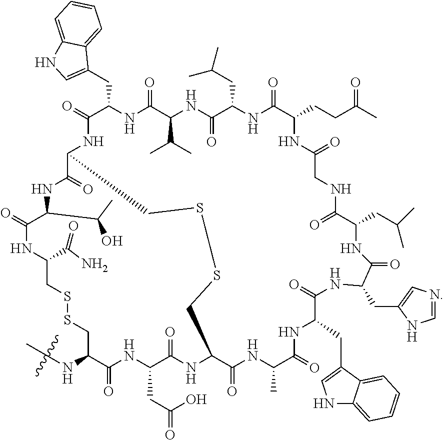 [SMILES: *N[C@H]1CSSC[C@@H](C(N)=O)NC(=O)[C@H]([C@@H](C)O)NC(=O)[C@@H]2CSSC[C@H](NC(=O)[C@H](CC(=O)O)NC1=O)C(=O)N[C@@H](C)C(=O)N[C@@H](Cc1c[nH]c3ccccc13)C(=O)N[C@@H](Cc1cnc[nH]1)C(=O)N[C@@H](CC(C)C)C(=O)NCC(=O)N[C@@H](CCC(C)=O)C(=O)N[C@@H](CC(C)C)C(=O)N[C@@H](C(C)C)C(=O)N[C@@H](Cc1c[nH]c3ccccc13)C(=O)N2]

In some embodiments, a antibody binding moiety is or comprises

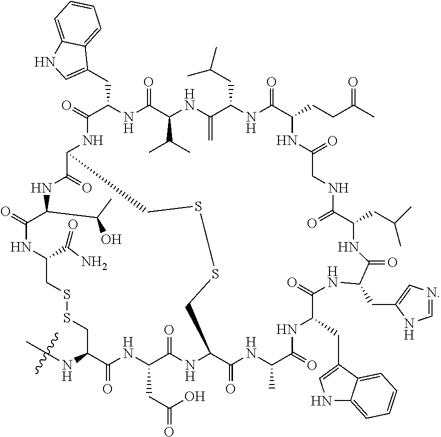 [SMILES: *N[C@H]1CSSC[C@@H](C(N)=O)NC(=O)[C@H]([C@@H](C)O)NC(=O)[C@@H]2CSSC[C@H](NC(=O)[C@H](CC(=O)O)NC1=O)C(=O)N[C@@H](C)C(=O)N[C@@H](Cc1c[nH]c3ccccc13)C(=O)N[C@@H](Cc1cnc[nH]1)C(=O)N[C@@H](CC(C)C)C(=O)NCC(=O)N[C@@H](CCC(C)=O)C(=O)N[C@@H](CC(C)C)C(=C)N[C@@H](C(C)C)C(=O)N[C@@H](Cc1c[nH]c3ccccc13)C(=O)N2]

In some embodiments, a antibody binding moiety is or comprises

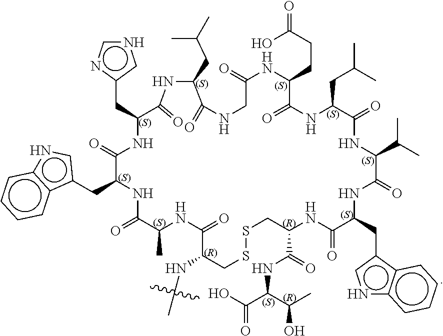

In some embodiments, a antibody binding moiety is or comprises

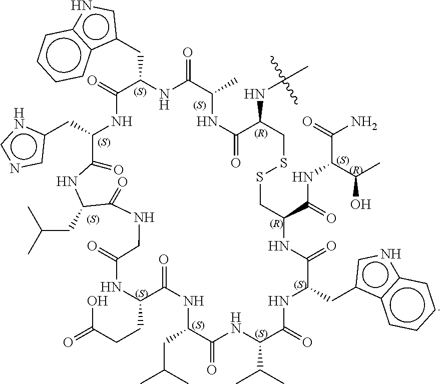

n some embodiments, a antibody binding moiety is or comprises

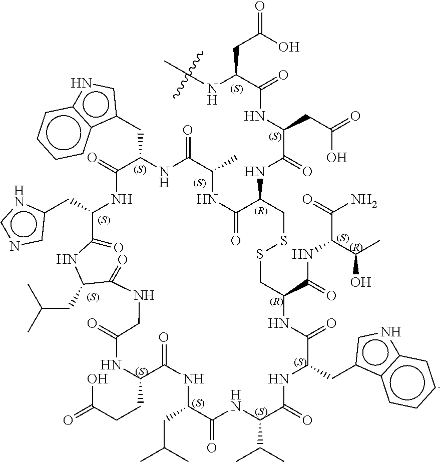

In some embodiments, a antibody binding moiety is or comprises

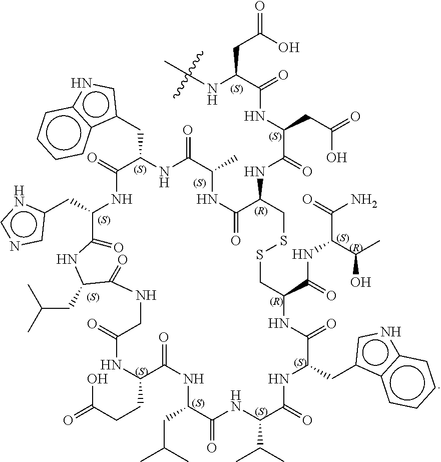

In some embodiments, a antibody binding moiety is or comprises

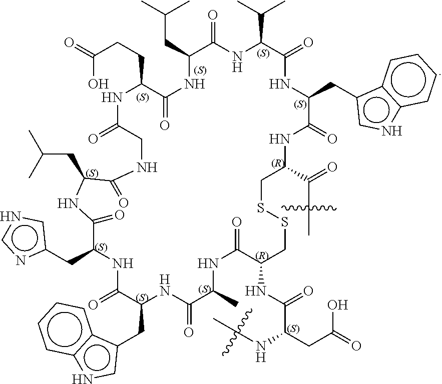

In some embodiments, a universal antibody binding moiety comprises a peptide unit, and is connected to a linker moiety through the C-terminus of the peptide unit. In some embodiments, it is connected to a linker moiety through the N-terminus of the peptide unit. In some embodiments, it is connected to a linker through a side chain group of the peptide unit. In some embodiments, a universal antibody binding moiety comprises a peptide unit, and is connected to a target binding moiety optionally through a linker moiety through the C-terminus of the peptide unit. In some embodiments, a universal antibody binding moiety comprises a peptide unit, and is connected to a target binding moiety optionally through a linker moiety through the N-terminus of the peptide unit. In some embodiments, a universal antibody binding moiety comprises a peptide unit, and is connected to a target binding moiety optionally through a linker moiety through a side chain of the peptide unit.

In some embodiments, an antibody binding moiety, e.g., a universal antibody binding moiety, is or comprises a small molecule entity, with a molecular weight of, e.g., less than 10000, 9000, 8000, 7000, 6000, 5000, 4000, 3000, 2000, 1500, 1000, etc. Suitable such antibody binding moieties include small molecule Fc binder moieties, e.g., those described in U.S. Pat. No. 9,745,339, US 201/30131321, etc. In some embodiments, an antibody binding moiety is of such a structure that its corresponding compound is a compound described in U.S. Pat. No. 9,745,339 or US 2013/0131321, the compounds of each of which are independently incorporated herein by reference. In some embodiments, ABT is of such a structure that H-ABT is a compound described in U.S. Pat. No. 9,745,339 or US 2013/0131321, the compounds of each of which are independently incorporated herein by reference. In some embodiments, such a compound can bind to an antibody. In some embodiments, such a compound can bind to Fc region of an antibody.

In some embodiments, an antibody binding moiety, e.g., an ABT is or comprises optionally substituted

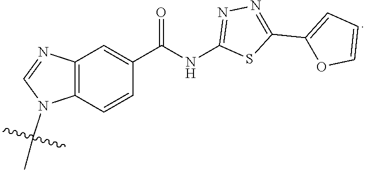 [SMILES: *n1cnc2cc(C(=O)Nc3nnc(-c4ccco4)s3)ccc21]

In some embodiments, an ABT is or comprises

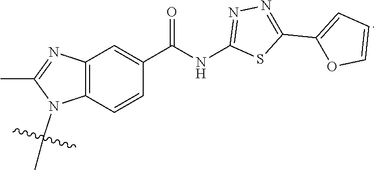 [SMILES: *n1c(C)nc2cc(C(=O)Nc3nnc(-c4ccco4)s3)ccc21]

In some embodiments, an ABT is or comprises optionally substituted

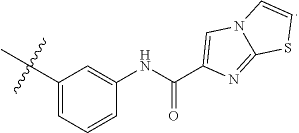 [SMILES: *c1cccc(NC(=O)c2cn3ccsc3n2)c1]

In some embodiments, an ABT is or comprises

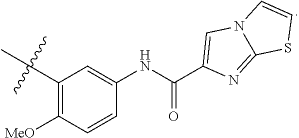 [SMILES: *c1cc(NC(=O)c2cn3ccsc3n2)ccc1OC]

In some embodiments, an ABT is or comprises optionally substituted

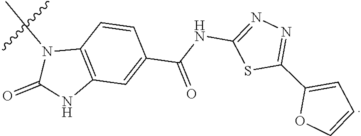 [SMILES: *n1c(=O)[nH]c2cc(C(=O)Nc3nnc(-c4ccco4)s3)ccc21]

In some embodiments, an ABT is or comprises

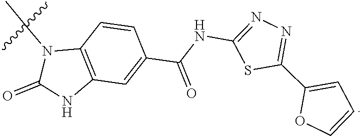 [SMILES: *n1c(=O)[nH]c2cc(C(=O)Nc3nnc(-c4ccco4)s3)ccc21]

In some embodiments, an ABT is or comprises optionally substituted

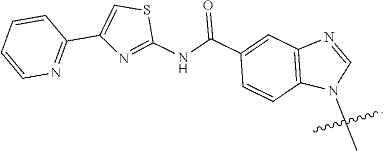 [SMILES: *n1cnc2cc(C(=O)Nc3nc(-c4ccccn4)cs3)ccc21]

In some embodiments, an ABT is or comprises

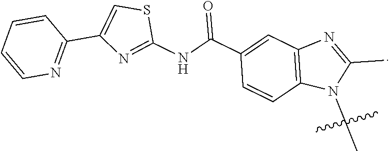 [SMILES: *n1c(C)nc2cc(C(=O)Nc3nc(-c4ccccn4)cs3)ccc21]

In some embodiments, an antibody binding moiety is a triazine moiety, e.g., one described in US 2009/0286693. In some embodiments, an antibody binding moiety is of such a structure that its corresponding compound is a compound described in US 2009/0286693, the compounds of which are independently incorporated herein by reference. In some embodiments, ABT is of such a structure that H-ABT is a compound described in US 2009/0286693, the compounds of which are independently incorporated herein by reference. In some embodiments, such a compound can bind to an antibody. In some embodiments, such a compound can bind to Fc region of an antibody.

In some embodiments, an antibody binding moiety is a triazine moiety, e.g., one described in Teng, et al., A strategy for the generation of biomimetic ligands for affinity chromatography. Combinatorial synthesis and biological evaluation of an IgG binding ligand, J. Mol. Recognit. 1999; 12:67-75 (“Teng”). In some embodiments, an antibody binding moiety is of such a structure that its corresponding compound is a compound described in Teng, the compounds of which are independently incorporated herein by reference. In some embodiments, ABT is of such a structure that H-ABT is a compound described in Teng, the compounds of which are independently incorporated herein by reference. In some embodiments, such a compound can bind to an antibody. In some embodiments, such a compound can bind to Fc region of an antibody.

In some embodiments, an antibody binding moiety is a triazine moiety, e.g., one described in Uttamchandani, et al., Microarrays of Tagged Combinatorial Triazine Libraries in the Discovery of Small-Molecule Ligands of Human IgG, J Comb Chem. 2004 November-December; 6(6):862-8 (“Uttamchandani”). In some embodiments, an antibody binding moiety is of such a structure that its corresponding compound is a compound described in Uttamchandani, the compounds of which are independently incorporated herein by reference. In some embodiments, ABT is of such a structure that H-ABT is a compound described in Uttamchandani, the compounds of which are independently incorporated herein by reference. In some embodiments, such a compound can bind to an antibody. In some embodiments, such a compound can bind to Fc region of an antibody.

In some embodiments, an antibody binding moiety binds to one or more binding sites of protein A. In some embodiments, an antibody binding moiety binds to one or more binding sites of protein G. In some embodiments, an antibody binding moiety binds to one or more binding sites of protein L. In some embodiments, an antibody binding moiety binds to one or more binding sites of protein Z. In some embodiments, an antibody binding moiety binds to one or more binding sites of protein LG. In some embodiments, an antibody binding moiety binds to one or more binding sites of protein LA. In some embodiments, an antibody binding moiety binds to one or more binding sites of protein AG. In some embodiments, an antibody binding moiety is described in Choe, W., Durgannavar, T. A., & Chung, S. J. (2016). Fc-binding ligands of immunoglobulin G: An overview of high affinity proteins and peptides. Materials, 9(12). https://doi.org/10.3390/ma9120994.

In some embodiments, an antibody binding moiety can bind to a nucleotide-binding site. In some embodiments, an antibody binding moiety is a small molecule moiety that can bind to a nucleotide-binding site. In some embodiments, a mall molecule is tryptamine. In some embodiments, ABT is of such a structure that H-ABT is tryptamine. Certain useful technologies were described in Mustafaoglu, et al., Antibody Purification via Affinity Membrane Chromatography Method Utilizing Nucleotide Binding Site Targeting With A Small Molecule, Analyst. 2016 November 28; 141(24): 6571-6582.

Many technologies are available for identifying and/or assessing and/or characterizing antibody binding moieties, including universal antibody binding moieties, and/or their utilization in ARMs, e.g., those described in WO/2019/023501, the technologies of which are incorporated herein by reference. In some embodiments, an antibody binding moiety is a moiety (e.g., small molecule moiety, peptide moiety, nucleic acid moiety, etc.) that can selectively bind to IgG, and when used in an ARM can provide and/or stimulate ADCC and/or ADCP. In some embodiments, peptide display technologies (e.g., phase display, non-cellular display, etc.) can be utilized to identify antibody binding moieties. In some embodiments, an antibody binding moiety is a moiety (e.g., small molecule moiety, peptide moiety, nucleic acid moiety, etc.) that can bind to IgG and optionally can compete with known antibody binders, e.g., protein A, protein G, protein L, etc.

As appreciated by those skilled in the art, antibodies of various properties and activities (e.g., antibodies recognizing different antigens, having optional modifications, etc.) may be recruited by antibody binding moieties described in the present disclosure. In some embodiments, such antibodies include antibodies administered to a subject, e.g., for therapeutic purposes. In some embodiments, antibodies recruited by antibody binding moieties comprise antibodies toward different antigens. In some embodiments, antibodies recruited by antibody binding moieties comprise antibodies whose antigens are not present on the surface or cell membrane of target cells (e.g., target cells such as cancer cells). In some embodiments, antibodies recruited by antibody binding moieties comprise antibodies which are not targeting antigens present on surface or cell membrane of targets (e.g., target cells such as cancer cells). In some embodiments, antigens on surface of target cells may interfere with the structure, conformation, and/or one or more properties and/or activities of recruited antibodies which bind such antigens. In some embodiments, as appreciated by those skilled in the art, provided technologies comprise universal antibody binding moieties which recruit antibodies of diverse specificities, and no more than 1%, 2%, 5%, 10%, 15%, 20%, 25%, 30%, 35%, 40%, 45%, 50%, 55%, 60%, 65%, 70%, 75%, 80%, 85%, 90%, 95% or 99% percent of recruited antibodies are toward the same antigen, protein, lipid, carbohydrate, etc. Among other things, one advantage of the present disclosure is that provided technologies comprising universal antibody binding moieties can utilize diverse pools of antibodies such as those present in serum. In some embodiments, universal antibody binding moieties of the present disclosure (e.g., those in ARMs) are contacted with a plurality of antibodies, wherein no more than 1%, 2%, 5%, 10%, 15%, 20%, 25%, 30%, 35%, 40%, 45%, 50%, 55%, 60%, 65%, 70%, 75%, 80%, 85%, 90%, 95% or 99% percent of the plurality of antibodies are toward the same antigen, protein, lipid, carbohydrate, etc.

### CD38

CD38 (cluster of differentiation 38, also known as cyclic ADP ribose hydrolase) is expressed by various types of cells and performs a number of functions. It has been reported that CD38 was found on the surface of many immune cells, e.g., CD4⁺, CD8⁺, B lymphocytes and natural killer cells, etc., as a glycoprotein. Other functions, e.g., cell adhesion, signal transduction and calcium signaling were also reported for CD38. In various embodiments, CD38 is a human CD38.

In some embodiments, it is reported CD38 is a non-lineage-restricted, type II transmembrane glycoprotein that synthesizes and hydrolyzes cyclic adenosine 5′-diphosphate-ribose, an intracellular calcium ion mobilizing messenger. Reportedly, the release of soluble protein and the ability of membrane-bound protein to become internalized may indicate both extracellular and intracellular functions for the protein. According to certain reports, in various cases CD38 has an N-terminal cytoplasmic tail, a single membrane-spanning domain, and a C-terminal extracellular region with four N-glycosylation sites. There are reports that crystal structure analysis demonstrates that the functional molecule is a dimer, with the central portion containing the catalytic site. It has been reported that CD38 may be used as a prognostic marker for patients with chronic lymphocytic leukemia. Alternative splicing has been reported and may result in multiple transcript variants.

It is reported that CD38 are expressed on immune system cells such as T cells or B cells of healthy person. In some embodiments, in certain conditions, disorders or diseases increased levels of CD38 expression and/or activities are observed in cells which typically do not have or have lower levels of CD38.

CD38 is associated with various conditions, disorders or diseases, e.g., HIV infection and various cancers, such as leukemia, myelomas, solid tumors, chronic lymphocytic leukemia (CLL), multiple myeloma (MM), Acute promyelocytic leukemia (APL), non-Hodgkin's lymphoma, B and T cell acute lymphotic leukemia, Acute myeloid leukemia, Hodgkin's lymphoma, chronic myeloid leukemia, etc.

Daratumumab, an antibody which targets CD38, has been approved in treating multiple myeloma.

### Target Binding Moieties

Target binding moieties of various types and chemical classes can be utilized in accordance with the present disclosure. Among other things, compounds of the present disclosure comprise target binding moieties that can bind to CD38. In some embodiments, target binding moieties bind to characteristic agents such as CD38. In some embodiments, target binding moieties are or comprise peptide moieties. In some embodiments, target binding moieties are or comprise nucleic acid agents such as aptamers. In some embodiments, target binding moieties are or comprise lipid moieties. Certain types of target binding moieties are described below; those skilled in the art appreciates that other types of target binding moieties, including many known in the art, can also be utilized in accordance with the present disclosure. As appreciated by those skilled in the art, various technologies are readily available and can be utilized to assess and confirm CD38 binding. Certain useful technologies are described in the Examples.

a. Small Molecules

In some embodiments, a target binding moiety is a small molecule moiety. In some embodiments, a small molecule moiety has a molecular weight no more than 8000, 7000, 6000, 5000, 4000, 3000, 2000, 1500, 1000, 900, 800, 700, or 600. In some embodiments, a small molecule moiety has a molecular weight no more than 8000. In some embodiments, a small molecule moiety has a molecular weight no more than 7000. In some embodiments, a small molecule moiety has a molecular weight no more than 6000. In some embodiments, a small molecule moiety has a molecular weight no more than 5000. In some embodiments, a small molecule moiety has a molecular weight no more than 4000. In some embodiments, a small molecule moiety has a molecular weight no more than 3000. In some embodiments, a small molecule moiety has a molecular weight no more than 2000. In some embodiments, a small molecule moiety has a molecular weight no more than 1500. In some embodiments, a small molecule moiety has a molecular weight no more than 1000. In some embodiments, a small molecule moiety has a molecular weight no more than 900. Among other things, the present disclosure encompasses the recognition that small molecule target binding moieties may be able to bind to markers, e.g., CD38, outside of, on the surface of, and/or inside of targets, e.g., cancer cells.

In some embodiments, a small molecule target binding moiety is or comprises a moiety that selectively binds to a protein or a fragment thereof, e.g., CD38.

b. Peptide Agents

In some embodiments, a target binding moiety is or comprises a peptide agent. In some embodiments, a target binding moiety is a peptide moiety. In some embodiments, a peptide moiety can either be linier or cyclic. In some embodiments, a target binding moiety is or comprises a cyclic peptide moiety. Various peptide target binding moieties are known in the art and can be utilized in accordance with the present disclosure.

In some embodiments, a target binding moiety is or comprises a peptide aptamer agent.

c. Aptamer Agents

In some embodiments, a target binding moiety is or comprises a nucleic acid agent. In some embodiments, a target binding moiety is or comprises an oligonucleotide moiety. In some embodiments, a target binding moiety is or comprises an aptamer agent. Various aptamer agents are known in the art or can be readily developed using common technologies, and can be utilized in provided technologies in accordance with the present disclosure.

In some embodiments, a target binding moiety, e.g., one that can bind to CD38, is or comprises a peptide moiety. In some embodiments, a peptide moiety is or comprises (Xaa)y or a salt form thereof as described herein. In some embodiments, a target binding moiety, e.g., one that can bind to CD38, is or comprises a peptide moiety, e.g., a moiety having the structure of:

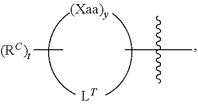

or a salt thereof, wherein:

each Xaa is independently a residue of an amino acid or an amino acid analog;

y is 5-20;

L^{T} is a linker moiety linking two residues each independently of an amino acid or an amino acid analog, and is independently a covalent bond, or an optionally substituted bivalent group selected from C₁-C₆ aliphatic or C₁-C₆ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;

each R^{c} is independently -L^{a}-R′;

t is 0-50;

each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;

each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;

each R′ is independently —R, —C(O)R, —CO₂R, or —SO₂R;

each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, is

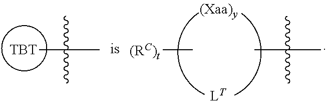

In some embodiments, each Xaa is independently an amino acid residue. In some embodiments, a Xaa is an amino acid analog residue. In some embodiments, one or more Xaa are independently a natural amino acid residue. In some embodiments, one or more Xaa are independently an unnatural amino acid residue. In some embodiments, side chains of two or more amino acid residues may be linked together to form bridges. In some embodiments, R^{c} and an amino acid residue side chain may be linked together to form a bridge. In some embodiments, each bridge independently has the structure of L^{a}. L^{b}, or L^{T}. In some embodiments, each bridge independently has the structure of L^{a}. In some embodiments, each bridge independently has the structure of L^{b}. In some embodiments, each bridge independently has the structure of L^{T}. For example, in some embodiments, side chains of two cysteine residues may form a disulfide bridge comprising —S—S— (which, as in many proteins, can be formed by two —SH groups). In some embodiments, R^{c} is -L^{a}-R, a Xaa is a residue of an amino acid having the structure of formula A-I wherein one of R^{a2} and R^{a3} is R, and the R of R^{c} is taken together with one of R^{a2} and R^{a3} to form a covalent bond.

In some embodiments, -(Xaa)y- is or comprises -Xaa^{T1}-Xaa^{T2}-(Xaa)y′-Xaa^{T3}-Xaa^{T4}-Xaa^{T5}-, wherein:

y′ is 0-8;

Xaa^{T1} is a residue of an amino acid or an amino acid analog whose side chain is substituted C₁-C₈ aliphatic;

Xaa^{T2} is a residue of an amino acid or an amino acid analog whose side chain comprises an optionally substituted aromatic group or is optionally substituted C₃-C₈ aliphatic;

Xaa^{T3} is a residue of an amino acid or an amino acid analog whose side chain is optionally substituted C₂-C₈ aliphatic;

Xaa^{T4} is a residue of an amino acid or an amino acid analog whose side chain comprises an optionally substituted aromatic group, or is optionally substituted C₃-C₈ aliphatic; and

Xaa^{T5} is a residue of an amino acid or an amino acid analog whose side chain is substituted C₁-C₈ aliphatic.

In some embodiments, y′ is 0. In some embodiments, y′ is 1. In some embodiments, y′ is 2. In some embodiments, y′ is 3. In some embodiments, y′ is 4. In some embodiments, y′ is 5. In some embodiments, y′ is 6. In some embodiments, y′ is 7. In some embodiments, y′ is 8.

In some embodiments, Xaa^{T1} comprises substituted C₁-C₈ aliphatic. In some embodiments, the side chain of Xaa^{T1} is or comprises optionally substituted C₂-C₈ aliphatic. In some embodiments, the side chain of Xaa^{T1} is or comprises optionally substituted C₂-C₈ alkyl. In some embodiments, the side chain of Xaa^{T1} is a C₂-C₈ alkyl. In some embodiments, the side chain of Xaa^{T1} is or comprises optionally substituted linear C₂-C₈ alkyl. In some embodiments, the side chain of Xaa^{T1} is a linear C₂-C₈ alkyl. In some embodiments, the side chain is n-pentyl. In some embodiments, Xaa^{T1} is (S)—NH—CH(n-C₅H₁₁)—C(O)—. In some embodiments, the side chain of Xaa^{T1} is or comprises an aromatic group. In some embodiments, the side chain is —CH₂—R, wherein the —CH₂— is optionally substituted, and R is an optionally substituted aryl or heteroaryl. In some embodiments, the side chain is the side chain of Y, W, S, K or K(MePEG4c). In some embodiments, the side chain is the side chain of Y, W or S. In some embodiments, Xaa^{T1} is a residue of Y. In some embodiments, Xaa^{T1} is a residue of W. In some embodiments, Xaa^{T1} embodiments, Xaa^{T1} is a residue of K. In some embodiments, Xaa^{T1} is a residue of K(MePEG4c).

In some embodiments, Xaa^{T2} is or comprises an aromatic group. In some embodiments, the side chain of Xaa^{T2} is —CH₂—R, wherein the —CH₂— is optionally substituted, and R is as described herein. In some embodiments, R is an optionally substituted aryl or heteroaryl. In some embodiments, R is optionally substituted phenyl. In some embodiments, R is 4-hydroxyphenyl. In some embodiments, R is 4-phenylphenyl. In some embodiments, the side chain is that of Y or W. In some embodiments, Xaa^{T2} is a residue of Y. In some embodiments, Xaa^{T2} is a residue of W. In some embodiments, Xaa^{T2} is a residue of Bph. In some embodiments, Xaa^{T2} comprises substituted C₁-C₈ aliphatic. In some embodiments, the side chain of Xaa^{T2} is or comprises optionally substituted C₂-C₈ aliphatic. In some embodiments, the side chain of Xaa^{T2} is or comprises optionally substituted C₃-C₈ aliphatic. In some embodiments, the side chain of Xaa^{T2} is or comprises optionally substituted C₂-C₈ alkyl. In some embodiments, the side chain of Xaa^{T2} is a C₂-C₈ alkyl. In some embodiments, the side chain of Xaa^{T2} is or comprises optionally substituted linear C₂-C₈ alkyl. In some embodiments, the side chain of Xaa^{T2} is a linear C₂-C₈ alkyl. In some embodiments, the side chain of Xaa^{T2} is a branched C₃-C₈ alkyl. In some embodiments, the side chain is n-pentyl. In some embodiments, Xaa^{T2} is (S)—NH—CH(n-C₅H₁₁)—C(O)—. In some embodiments, the side chain is (CH₃)₂CHCH₂—. In some embodiments, Xaa^{T2} is a residue of L. In some embodiments, Xaa^{T2} is a residue of A.

In some embodiments, Xaa^{T3} comprises substituted C₁-C₈ aliphatic. In some embodiments, the side chain of Xaa^{T3} is or comprises optionally substituted C₂-C₈ aliphatic. In some embodiments, the side chain of Xaa^{T3} is or comprises optionally substituted C₃-C₈ aliphatic. In some embodiments, the side chain of Xaa^{T3} is or comprises optionally substituted C₂-C₈ alkyl. In some embodiments, the side chain of Xaa^{T3} is a C₂-C₈ alkyl. In some embodiments, the side chain of Xaa^{T3} is or comprises optionally substituted linear C₂-C₈ alkyl. In some embodiments, the side chain of Xaa^{T3} is a linear C₂-C₈ alkyl. In some embodiments, the side chain of Xaa^{T3} is a branched C₃-C₈ alkyl. In some embodiments, the side chain is n-pentyl. In some embodiments, Xaa^{T3} is a residue of Ahp. In some embodiments, the side chain is that of L, V or T. In some embodiments, Xaa^{T3} is a residue of L. In some embodiments, Xaa^{T3} is a residue of V. In some embodiments, Xaa^{T3} is a residue of T. In some embodiments, Xaa^{T3} comprises a side chain comprising two or more sp³ carbon atoms. In some embodiments, Xaa^{T3} comprises a side chain comprising two or more groups each of which is independently —CH₂— or —CH₃. In some embodiments, Xaa^{T3} comprises a polar side chain, e.g., comprising —OH, —SO₂— etc. In some embodiments, Xaa^{T3} is a residue of Hse (homoserine). In some embodiments, Xaa^{T3} is a residue of MetO2 (methionine sulfone).

In some embodiments, Xaa^{T4} is or comprises an aromatic group. In some embodiments, the side chain of Xaa^{T4} is —CH₂—R, wherein the —CH₂— is optionally substituted, and R is as described herein. In some embodiments, R is an optionally substituted aryl or heteroaryl. In some embodiments, R is optionally substituted phenyl. In some embodiments, R is 4-hydroxyphenyl. In some embodiments, R is 4-phenylphenyl. In some embodiments, the side chain is that of Y or W. In some embodiments, Xaa^{T4} is a residue of Y. In some embodiments, Xaa^{T4} is a residue of W. In some embodiments, Xaa^{T4} is a residue of Bph. In some embodiments, Xaa^{T4} comprises substituted C₁-C₈ aliphatic. In some embodiments, the side chain of Xaa^{T4} is or comprises optionally substituted C₂-C₈ aliphatic. In some embodiments, the side chain of Xaa^{T4} is or comprises optionally substituted C₃-C₈ aliphatic. In some embodiments, the side chain of Xaa^{T4} is or comprises optionally substituted C₂-C₈ alkyl. In some embodiments, the side chain of Xaa^{T4} is a C₂-C₈ alkyl. In some embodiments, the side chain of Xaa^{T4} is or comprises optionally substituted linear C₂-C₈ alkyl. In some embodiments, the side chain of Xaa^{T4} is a linear C₂-C₈ alkyl. In some embodiments, the side chain of Xaa^{T4} is a branched C₃-C₈ alkyl. In some embodiments, the side chain is n-pentyl. In some embodiments, the side chain is isopropyl. In some embodiments, Xaa^{T4} is a residue of V. In some embodiments, Xaa^{T4} is a residue of Ahp.

In some embodiments, Xaa^{T5} comprises substituted C₁-C₂₀ aliphatic. In some embodiments, Xaa^{T5} comprises substituted C₁-C₁₅ aliphatic. In some embodiments, Xaa^{T5} comprises substituted C₁-C₁₀ aliphatic. In some embodiments, Xaa^{T5} comprises substituted C₁-C₈ aliphatic. In some embodiments, the side chain of Xaa^{T5} is or comprises optionally substituted C₂-C₈ aliphatic. In some embodiments, the side chain of Xaa^{T5} is or comprises optionally substituted C₂-C₈ alkyl. In some embodiments, the side chain of Xaa^{T5} is a C₂-C₈ alkyl. In some embodiments, the side chain of Xaa^{T5} is or comprises optionally substituted linear C₂-C₈ alkyl. In some embodiments, the side chain of Xaa^{T5} is a linear C₂-C₈ alkyl. In some embodiments, the side chain is n-pentyl. In some embodiments, Xaa^{T5} is (S)—NH—CH(n-C₅H₁₁)—C(O)—. In some embodiments, the side chain of Xaa^{T5} is or comprises an aromatic group. In some embodiments, the side chain is —CH₂—R, wherein the —CH₂— is optionally substituted, and R is an optionally substituted aryl or heteroaryl. In some embodiments, R is optionally substituted phenyl. In some embodiments, R is 4-phenylphenyl. In some embodiments, the side chain is the side chain of Y, W or S. In some embodiments, Xaa^{T5} is a residue of Y. In some embodiments, Xaa^{T5} is a residue of W. In some embodiments, Xaa^{T5} is a residue of Bph. In some embodiments, Xaa^{T5} is a residue of S. In some embodiments, Xaa^{T5} is a residue of Ado. In some embodiments, Xaa^{T5} is a residue of Ano. In some embodiments, Xaa^{T5} is a residue of PhNle. In some embodiments, Xaa^{T5} is a residue of PhNva.

In some embodiments, the side chain of Xaa^{T1} is or comprises the side chain of Ahp or Y. In some embodiments, the side chain of Xaa^{T2} is or comprises the side chain of Y, W, Ahp or Bph. In some embodiments, the side chain of Xaa^{T3} is or comprises the side chain of L, C or Ahp. In some embodiments, the side chain of Xaa^{T4} is or comprises the side chain of Bph or V. In some embodiments, the side chain of Xaa^{T5} is or comprises the side chain of Ahp or Bph.

In some embodiments, Xaa is a residue of Ahp or Y. In some embodiments, Xaa^{T2} is a residue of Y, W, Ahp or Bph. In some embodiments, Xaa^{T3} is a residue of L, C or Ahp. In some embodiments, Xaa^{T4} is a residue of Bph or V. In some embodiments, Xaa^{T5} is a residue of Ahp or Bph.

In some embodiments, -(Xaa)y- is or comprises:

-(Xaa)ₐ₁-(Xaa)ₐ₂-(Xaa)ₐ₃-(Xaa)ₐ₄-(Xaa)ₐ₅-(Xaa)ₐ₆-(Xaa)ₐ₇-(Xaa)ₐ₈-(Xaa)ₐ₉-(Xaa)ₐ₁₀-(Xaa)ₐ₁₁-(Xaa)ₐ₁₂-(Xaa)ₐ₁₃-,

wherein:

each of a1, a2, a3, a4, a5, a6, a7, a8, a9, a10, a11, a12 and a13 is independently 0-5;

(Xaa)ₐ₃ is or comprises Xaa^{T1};

(Xaa)ₐ₄ is or comprises Xaa^{T2};

(Xaa)ₐ₉ is or comprises Xaa^{T3};

(Xaa)ₐ₁₀ is or comprises Xaa^{T4}; and

(Xaa)ₐ₁₁ is or comprises Xaa^{T5}

In some embodiments, a1, a2, a3, a4, a5, a6, a7, a8, a9, a10, a11, a12, and/or a13 is independently 0. In some embodiments, a1, a2, a3, a4, a5, a6, a7, a8, a9, a10, a11, a12, and/or a13 is independently 1. In some embodiments, a1, a2, a3, a4, a5, a6, a7, a8, a9, a10, a11, a12, and/or a13 is independently 2. In some embodiments, a1, a2, a3, a4, a5, a6, a7, a8, a9, a10, a11, a12, and/or a13 is independently 3. In some embodiments, a1, a2, a3, a4, a5, a6, a7, a8, a9, a10, a11, a12, and/or a13 is independently 4. In some embodiments, a1, a2, a3, a4, a5, a6, a7, a8, a9, a10, a11, a12, and/or a13 is independently 5. In some embodiments, a1, a2, a3, a4, a5, a6, a7, a8, a9, a10, a11, a12, and a13 is 1.

In some embodiments, (Xaa)ₐ₁ is or comprises A. In some embodiments, a1 is 1 and (Xaa)ₐ₁ is a residue of A. Other residues may also be utilized. For example, in some embodiments, (Xaa)ₐ₁ is or comprises K. In some embodiments, K is connected to another moiety, e.g., an antibody binding moiety optionally through a linker. In some embodiments, (Xaa)ₐ₁ is or comprises K(MePEG4c) (CH₃O(CH₂CH₂O)₃CH₂CH₂C(O)— bonded to the amino group in the side chain of K; see exemplary structures as described herein). In some embodiments, a1 is 0. In some embodiments, a Xaa of (Xaa)ₐ₁ (e.g., a N-terminal residue) is linked to another Xaa (e.g., of a Xaa of (Xaa)ₐ₁₃, of a C-terminal residue, etc.) as described herein. For example, in some embodiments, a N-terminal residue is linked via its amino group to a C-terminal cysteine via its —S— through a linker (e.g., —C(O)—CH₂—, wherein the —C(O)— is bonded to the amino group and the —CH₂— is bonded to the —S—).

In some embodiments, (Xaa)ₐ₂ is or comprises a residue whose side chain comprises a heteroatom, OH or NH. In some embodiments, (Xaa)ₐ₂ is or comprises a residue that comprises a polar or charged side chain. Other types of residues can also be utilized, e.g., those comprising hydrophobic aliphatic side chains such as A. In some embodiments, (Xaa)ₐ₂ is or comprises a residue whose side chain is the side chain of R, S, D, Y, A, W, K, 4Py2NH2 ((S)-2-amino-3-(2-aminopyridin-4-yl) propanoic acid), Cit (Citrulline), F3G (3-Guanidinophenylalanine), hCit (2-Amino-5-(carbamoylamino) hexanoic acid; CAS: 201485-17-8), K(MePEG4c), RNdMe (N⁵-[(dimethylamino)iminomethyl]-L-ornithine; CAS: 1185841-84-2), RNMe (N⁵-[imino(methylamino)methyl]-acetate-L-ornithine; CAS: 1135616-49-7), or RNNdMe (N⁵-[(methylamino)(methylimino)methyl]-L-ornithine). In some embodiments, (Xaa)ₐ₂ is or comprises a residue whose side chain is the side chain of R, S, D, Y, A or W. In some embodiments, (Xaa)ₐ₂ is or comprises a residue of R, S, D, Y, A, W, K, 4Py2NH2, Cit, F3G, hCit, K(MePEG4c), RNdMe, RNMe, or RNNdMe. In some embodiments, (Xaa)ₐ₂ is or comprises a residue of R, S, D, Y, A, or W. In some embodiments, (Xaa)ₐ₂ is or comprises R. In some embodiments, (Xaa)ₐ₂ is or comprises S. In some embodiments, (Xaa)ₐ₂ is or comprises D. In some embodiments, (Xaa)ₐ₂ is or comprises Y. In some embodiments, (Xaa)ₐ₂ is or comprises W. In some embodiments, (Xaa)ₐ₂ is or comprises A. In some embodiments, (Xaa)ₐ₂ is or comprises S. In some embodiments, (Xaa)ₐ₂ is or comprises K. In some embodiments, (Xaa)ₐ₂ is or comprises 4Py2NH2. In some embodiments, (Xaa)ₐ₂ is or comprises Cit. In some embodiments, (Xaa)ₐ₂ is or comprises F3G. In some embodiments, (Xaa)ₐ₂ is or comprises hCit. In some embodiments, (Xaa)ₐ₂ is or comprises K(MePEG4c). In some embodiments, (Xaa)ₐ₂ is or comprises RNdMe. In some embodiments, (Xaa)ₐ₂ is or comprises RNMe. In some embodiments, (Xaa)ₐ₂ is or comprises RNNdMe. In some embodiments, a2 is 1.

In some embodiments, a3 is 1. In some embodiments, (Xaa)ₐ₃ is Xaa^{T1} as described herein.

In some embodiments, a4 is 1. In some embodiments, (Xaa)ₐ₄ is Xaa^{T2} as described herein.

In some embodiments, (Xaa)ₐ₅ is or comprises Xaa^{T1}. In some embodiments, (Xaa)ₐ₅ is or comprises a residue whose side chain comprises an aromatic group. In some embodiments, (Xaa)ₐ₅ is or comprises a residue whose side chain comprises a heteroatom, OH or NH. In some embodiments, (Xaa)ₐ₅ is or comprises a residue that comprises a polar or charged side chain. Other types of residues can also be utilized, e.g., those comprising hydrophobic aliphatic side chains such as A. In some embodiments, (Xaa)ₐ₅ is or comprises a residue whose side chain is the side chain of H, A, Y, S, L, W or W6N. In some embodiments, (Xaa)ₐ₅ is or comprises a residue whose side chain is the side chain of H, A, Y, S, L or W. In some embodiments, (Xaa)ₐ₅ is or comprises a residue of H, A, Y, S, L, W or W6N. In some embodiments, (Xaa)ₐ₅ is or comprises a residue of H, A, Y, S, L or W. In some embodiments, (Xaa)ₐ₅ is or comprises H. In some embodiments, (Xaa)ₐ₅ is or comprises A. In some embodiments, (Xaa)ₐ₅ is or comprises Y. In some embodiments, (Xaa)ₐ₅ is or comprises S. In some embodiments, (Xaa)ₐ₅ is or comprises L. In some embodiments, (Xaa)ₐ₅ is or comprises W. In some embodiments, (Xaa)ₐ₅ is or comprises W6N. In some embodiments, a5 is 1.

In some embodiments, (Xaa)ₐ₆ is or comprises a residue that comprises a polar or charged side chain. Other types of residues can also be utilized, e.g., those comprising hydrophobic aliphatic side chains such as A. In some embodiments, (Xaa)ₐ₆ is or comprises a residue that comprises no side chain. In some embodiments, (Xaa)ₐ₆ is or comprises a residue whose side chain is the side chain of D, R, A or Y. In some embodiments, (Xaa)ₐ₆ is or comprises a residue of D, A, G, R or Y. In some embodiments, (Xaa)ₐ₆ is or comprises D. In some embodiments, (Xaa)ₐ₆ is or comprises A. In some embodiments, (Xaa)ₐ₆ is or comprises G. In some embodiments, (Xaa)ₐ₆ is or comprises R. In some embodiments, (Xaa)ₐ₆ is or comprises Y. In some embodiments, a6 is 1.

In some embodiments, (Xaa)ₐ₇ is or comprises a residue that comprises a polar or charged side chain. Other types of residues can also be utilized, e.g., those comprising hydrophobic aliphatic side chains such as A. In some embodiments, a polar or charged amino acid residue may provide certain benefits, e.g., improved solubility for manufacturing, administration, delivery, activity, etc. In some embodiments, (Xaa)ₐ₇ is or comprises a residue that comprises no side chain. In some embodiments, (Xaa)ₐ₇ is or comprises a residue whose side chain is the side chain of MetO2 (Methionine Sulfone), D, R, A or Y. In some embodiments, (Xaa)ₐ₇ is or comprises a residue whose side chain is the side chain of D, R, A or Y. In some embodiments, (Xaa)ₐ₇ is or comprises a residue whose side chain is the side chain of D, R, or S. In some embodiments, (Xaa)ₐ₇ is or comprises a residue whose side chain is the side chain of D, E, N or Q. In some embodiments, (Xaa)ₐ₇ is or comprises a residue of MetO2, D, A, G, R or Y. In some embodiments, (Xaa)ₐ₇ is or comprises a residue of D, A, G, R or Y. In some embodiments, (Xaa)ₐ₇ is or comprises a residue of D, E, N or Q. In some embodiments, (Xaa)ₐ₇ is or comprises a residue of D, G, R or S. In some embodiments, (Xaa)ₐ₇ is or comprises G. In some embodiments, (Xaa)ₐ₇ is or comprises MetO2. In some embodiments, (Xaa)ₐ₇ is or comprises A. In some embodiments, (Xaa)ₐ₇ is or comprises D. In some embodiments, (Xaa)ₐ₇ is or comprises E. In some embodiments, (Xaa)ₐ₇ is or comprises Q. In some embodiments, (Xaa)ₐ₇ is or comprises N. In some embodiments, (Xaa)ₐ₇ is or comprises R. In some embodiments, (Xaa)ₐ₇ is or comprises S. In some embodiments, a7 is 1.

In some embodiments, (Xaa)ₐ₈ is or comprises a residue that comprises a hydrophobic side chain. In some embodiments, (Xaa)ₐ₈ is or comprises a residue that comprises an aliphatic side chain. In some embodiments, (Xaa)ₐ₈ is or comprises a residue whose side chain is the side chain of V, D, G, W, S, T, or A. In some embodiments, (Xaa)ₐ₈ is or comprises a residue of V, D, G, W, S, T, or A. In some embodiments, (Xaa)ₐ₈ is or comprises V. In some embodiments, (Xaa)ₐ₈ is or comprises D. In some embodiments, (Xaa)ₐs is or comprises G. In some embodiments, (Xaa)ₐ₈ is or comprises W. In some embodiments, (Xaa)ₐ₈ is or comprises S. In some embodiments, (Xaa)ₐ₈ is or comprises T. In some embodiments, (Xaa)ₐ₈ is or comprises A. In some embodiments, a8 is 1.

In some embodiments, a9 is 1. In some embodiments, (Xaa)ₐ₉ is Xaa^{T3} as described herein.

In some embodiments, a10 is 1. In some embodiments, (Xaa)ₐ₁₀ is Xaa^{T4} as described herein.

In some embodiments, a11 is 1. In some embodiments, (Xaa)ₐ₁₁ is Xaa^{T5} as described herein.

In some embodiments, (Xaa)ₐ₁₂ is or comprises a residue that comprises a polar or charged side chain. In some embodiments, (Xaa)ₐ₁₂ is or comprises a residue that comprises no side chain. In some embodiments, (Xaa)ₐ₁₂ is or comprises a residue that comprises a hydrophobic side chain. In some embodiments, (Xaa)ₐ₁₂ is or comprises a residue whose side chain is the side chain of D, S, G, Ahp or A. In some embodiments, (Xaa)ₐ₁₂ is or comprises a residue of D, S, G, Ahp or A. In some embodiments, (Xaa)ₐ₁₂ is or comprises D. In some embodiments, (Xaa)ₐ₁₂ is or comprises S. In some embodiments, (Xaa)ₐ₁₂ is or comprises G. In some embodiments, (Xaa)ₐ₁₂ is or comprises Ahp. In some embodiments, (Xaa)ₐ₁₂ is or comprises A. In some embodiments, a12 is 1.

In some embodiments, (Xaa)ₐ₁₃ is or comprises a residue whose side chain comprises a nucleophile. In some embodiments, (Xaa)ₐ₁₃ is or comprises a residue whose side chain comprises —S—. In some embodiments, (Xaa)ₐ₁₃ is or comprises a residue whose side chain is the side chain of C. In some embodiments, (Xaa)ₐ₁₃ is or comprises a residue of C. In some embodiments, a13 is 1. In some embodiments, a13 is greater than 1, and the last residue is a residue whose side chain comprises a nucleophile as described herein, e.g., C. In some embodiments, a Xaa of (Xaa)ₐ₁₃ (e.g., a C-terminal residue) is linked to another Xaa (e.g., of a Xaa of (Xaa)ₐ₁, of a C-terminal residue, etc.) as described herein. In some embodiments, it is linked via a linker, e.g., L^{T} as described herein. For example, in some embodiments, a C-terminal residue is linked via its —S— to a N-terminal cysteine via its amino group through a linker (e.g., —C(O)—CH₂—, wherein the —C(O)— is bonded to the amino group and the —CH₂— is bonded to the —S—). In some embodiments, a residue whose side chain comprises —S— (e.g., of a residue of C) is linked to an amino group of another residue through a linker (e.g., —C(O)—CH₂—, wherein the —C(O)— is bonded to the amino group and the —CH₂— is bonded to the —S—).

Exemplary sequences and data thereof include those described below:

| | | | | | | | | | | | | | Frequency from | |
|---|---|---|---|---|---|---|---|---|---|---|---|---|---|---|
| 1 | 2 | 3 | 4 | 5 | 6 | 7 | 8 | 9 | 10 | 11 | 12 | 13 | Screening | |
| A | R | Ahp | Y | H | D | G | V | L | Bph | Ahp | D | C | | 14.0% |
| A | S | Y | Y | Y | G | D | V | L | Bph | Ahp | S | C | | 8.2% |
| A | D | Y | Y | Y | R | D | V | Ahp | Bph | Ahp | S | C | | 4.0% |
| A | R | Ahp | Y | H | G | D | V | L | Bph | Ahp | S | C | | 2.5% |
| A | D | Y | Y | S | G | D | V | L | Bph | Ahp | S | C | | 0.8% |
| A | R | Y | Y | L | D | G | D | L | Bph | Ahp | D | C | | 0.5% |
| A | R | Ahp | Y | H | D | G | V | L | Bph | Ahp | G | C | | 0.5% |
| A | Y | S | W | Y | Y | D | G | V | V | Bph | Ahp | T | C | 0.5% |
| A | D | Y | Y | Y | D | G | W | L | Bph | Ahp | S | C | | 0.4% |
| A | W | Y | Ahp | Y | D | G | S | V | Bph | Ahp | D | C | | 0.4% |
| A | R | Ahp | Bph | H | D | D | V | Ahp | Bph | Ahp | G | C | | 0.3% |
| A | S | W | Y | H | D | D | V | V | Bph | Ahp | S | C | | 0.3% |
| A | S | Y | Y | W | D | G | V | V | Bph | Ahp | S | C | | 0.3% |
| A | R | Ahp | Y | H | D | G | V | L | Bph | Ahp | S | C | | 0.3% |
| A | S | Y | Bph | H | D | G | V | T | Bph | Ahp | S | C | | 0.2% |
| A | R | Y | Y | H | R | D | S | L | Bph | Ahp | S | C | | 0.1% |
| A | R | Ahp | Ahp | Y | D | G | T | V | Bph | Ahp | D | C | | 0.1% |
| A | R | Ahp | Y | H | D | R | T | L | Bph | Ahp | G | C | | 0.1% |
| A | S | Ahp | Y | H | G | D | V | L | Bph | Ahp | S | C | | 0.1% |
| A | S | W | Y | Y | D | S | V | R | Bph | Ahp | S | C | | 0.1% |
| A | R | Ahp | Y | W | R | G | T | L | Bph | Ahp | D | C | | 0.1% |
| A | R | Y | Ahp | Y | D | S | V | V | Bph | Ahp | S | C | | 0.1% |

| 1 | 2 | 3 | 4 | 5 | 6 | 7 | 8 | 9 | 10 | 11 | 12 | 13 | Assay* |
|---|---|---|---|---|---|---|---|---|---|---|---|---|---|
| A | R | Ahp | Y | H | D | G | V | L | Bph | Ahp | D | C | 1.97 |
| A | A | Ahp | Y | H | D | G | V | L | Bph | Ahp | D | C | 0.60 |
| A | R | A | Y | H | D | G | V | L | Bph | Ahp | D | C | 0.11 |
| A | R | Ahp | A | H | D | G | V | L | Bph | Ahp | D | C | 0.11 |
| A | R | Ahp | Y | A | D | G | V | L | Bph | Ahp | D | C | 0.28 |
| A | R | Ahp | Y | H | A | G | V | L | Bph | Ahp | D | C | 0.93 |
| A | R | Ahp | Y | H | D | A | V | L | Bph | Ahp | D | C | 1.49 |
| A | R | Ahp | Y | H | D | G | A | L | Bph | Ahp | D | C | 0.30 |
| A | R | Ahp | Y | H | D | G | V | A | Bph | Ahp | D | C | 0.08 |
| A | R | Ahp | Y | H | D | G | V | L | A | Ahp | D | C | 0.09 |
| A | R | Ahp | Y | H | D | G | V | L | Bph | A | D | C | 0.20 |
| A | R | Ahp | Y | H | D | G | V | L | Bph | Ahp | A | C | 1.79 |

*Larger values represent binding. ELISA assay.

In some embodiments, a target binding moiety or

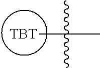

is as described above and/or as utilized in a compound in Table 1.

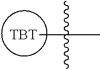

is or comprises

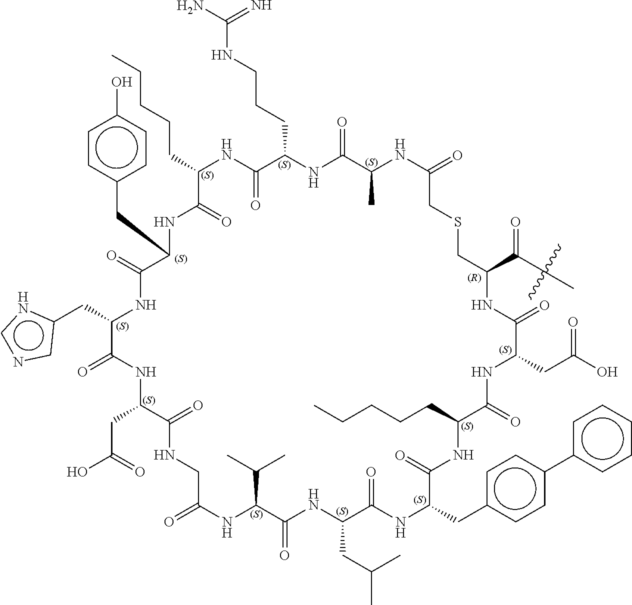

or a salt form thereof. In some embodiments,

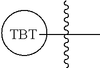

is or comprises

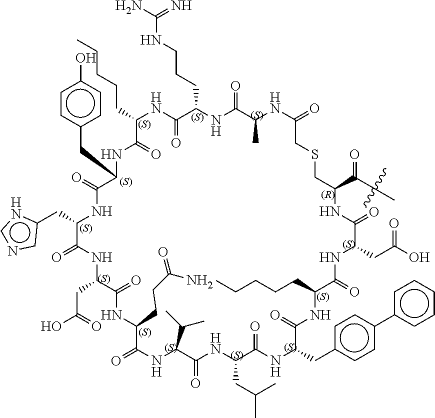

or a salt form thereof. In some embodiments,

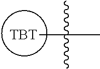

is or comprises

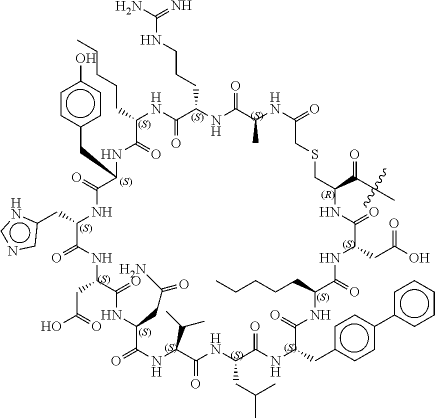

or a salt form thereof. In some embodiments,

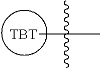

is or comprises

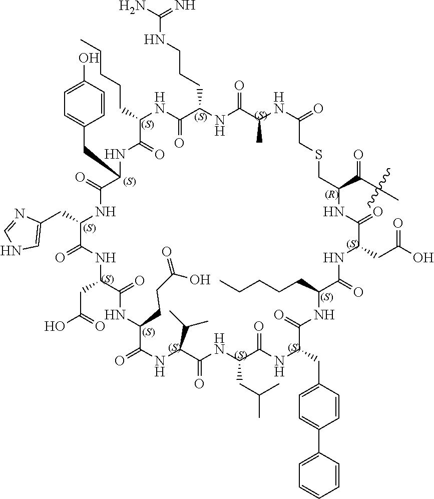

or a salt form thereof. In some embodiments,

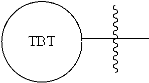

is or comprises

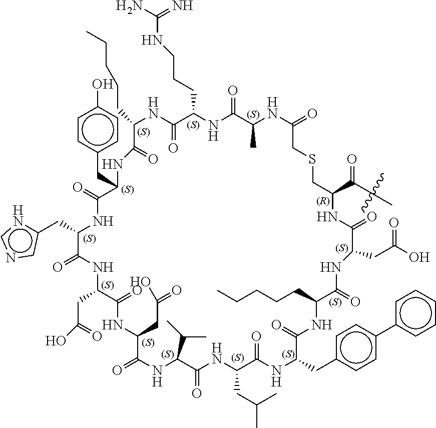

or a salt form thereof. In some embodiments,

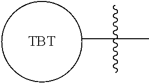

is or comprises

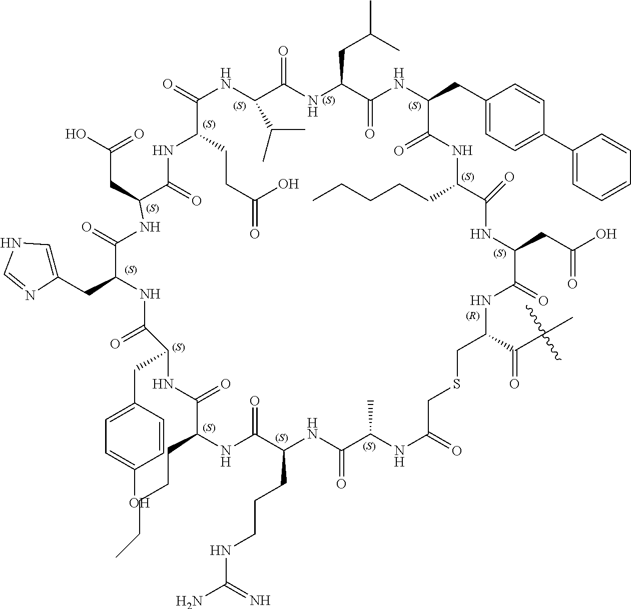

or a salt form thereof.

In some embodiments, -(Xaa)y- is or comprises -Xaa^{T6}-Xaa)y′-Xaa^{T7}-Xaa^{T8}-Xaa^{T9}-Xaa^{T10}-Xaa^{T11}-,

wherein:

y′ is 0-8;

Xaa^{T6} is a residue of an amino acid or an amino acid analog whose side chain is substituted C₁-C₈ aliphatic;

Xaa^{T7} is a residue of an amino acid or an amino acid analog whose side chain is optionally substituted C₂-C₈ aliphatic;

Xaa^{T8} is a residue of proline or an amino acid analog thereof;

Xaa^{T9} is a residue of an amino acid or an amino acid analog whose side chain comprises an optionally substituted aromatic group, or is optionally substituted C₁-C₈ aliphatic;

Xaa^{T10} is a residue of an amino acid or an amino acid analog whose side chain is substituted C₁-C₈ aliphatic, or an amino acid whose amino group is substituted; and

Xaa^{T11} is a residue of an amino acid or an amino acid analog whose side chain comprises an optionally substituted aromatic group, or is optionally substituted C₁-C₈ aliphatic.

In some embodiments, y′ is 0. In some embodiments, y′ is 1. In some embodiments, y′ is 2. In some embodiments, y′ is 3. In some embodiments, y′ is 4. In some embodiments, y′ is 5. In some embodiments, y′ is 6. In some embodiments, y′ is 7. In some embodiments, y′ is 8.

In some embodiments, Xaa^{T6} is a residue comprising a hydrophobic side chain. In some embodiments, Xaa^{T6} is a residue whose side chain comprises substituted C₁-C₈ aliphatic. In some embodiments, the side chain is —CH₂—R, wherein the —CH₂— is optionally substituted, and R is an optionally substituted aryl or heteroaryl. In some embodiments, R is phenyl. In some embodiments, the side chain is —CH₂Ph. In some embodiments, Xaa^{T6} is an amino acid residue, and its amino group is substituted. In some embodiments, its amino group is —N(R′)—. In some embodiments, R′ is optionally substituted C₁-C₆ alkyl. In some embodiments, R′ is methyl. In some embodiments, the side chain is the side chain of MeF (F wherein there is a methyl group on N (—N(Me)-), L, or S. In some embodiments, Xaa^{T6} is —N(Me)-CH(CH₂Ph)-C(O)—. In some embodiments, Xaa^{T6} is a residue of MeF, L or S.

In some embodiments, the side chain of Xaa^{T7} is or comprises optionally substituted C₂-C₈ aliphatic. In some embodiments, the side chain of Xaa^{T7} is or comprises optionally substituted C₂-C₈ alkyl. In some embodiments, the side chain of Xaa^{T7} is or comprises optionally substituted C₃-C₈ alkyl. In some embodiments, the side chain of Xaa^{T7} is or comprises optionally substituted C₄-C₈ alkyl. In some embodiments, the side chain of Xaa^{T7} is or comprises optionally substituted C₃-C₈ branched alkyl. In some embodiments, the side chain of Xaa^{T7} is or comprises optionally substituted C₄-C₈ branched alkyl. In some embodiments, the side chain of Xaa^{T7} is a branched C₃-C₈ alkyl. In some embodiments, the side chain of Xaa^{T7} is a branched C₄-C₈ alkyl. In some embodiments, the side chain is (CH₃)₂CHCH₂—. In some embodiments, the side chain is the side chain of L. In some embodiments, Xaa^{T7} is a residue of L.

In some embodiments, Xaa^{T8} is a residue comprising a cyclic moiety which participates in the backbone. In some embodiments, Xaa^{T8} is P.

In some embodiments, Xaa^{T9} is or comprises an aromatic group. In some embodiments, the side chain of Xaa^{T9} is —CH₂—R, wherein the —CH₂— is optionally substituted, and R is as described herein. In some embodiments, R is an optionally substituted aryl or heteroaryl. In some embodiments, R is optionally substituted phenyl. In some embodiments, R is 4-hydroxyphenyl. In some embodiments, R is 4-phenylphenyl. In some embodiments, the side chain is that of Bph. In some embodiments, Xaa^{T9} is a residue of Bph. In some embodiments, Xaa^{T9} comprises substituted C₁-C₈ aliphatic. In some embodiments, a substitution is a polar or charged group, such as —OH, —COOH, etc. In some embodiments, the side chain is that of D or S. In some embodiments, Xaa^{T9} is a residue of D or S.

In some embodiments, the side chain of Xaa^{T10} is or comprises substituted C₁-C₈ aliphatic. In some embodiments, the side chain of Xaa^{T10} is or comprises optionally substituted C₂-C₈ aliphatic. In some embodiments, the side chain of Xaa^{T10} is or comprises optionally substituted C₂-C₈ alkyl. In some embodiments, the side chain of Xaa^{T10} is a C₂-C₈ alkyl. In some embodiments, the side chain of Xaa^{T10} is or comprises optionally substituted linear C₂-C₈ alkyl. In some embodiments, the side chain of Xaa^{T10} is a linear C₂-C₈ alkyl. In some embodiments, the side chain of Xaa^{T10} is or comprises optionally substituted branched C₃-C₈ alkyl. In some embodiments, the side chain of Xaa^{T10} is a branched C₃-C₈ alkyl. In some embodiments, the side chain is (CH₃)₂CH—. In some embodiments, the side chain is (CH₃)₂CHCH₂—. In some embodiments, Xaa^{T10} is a residue of V. In some embodiments, Xaa^{T10} is a residue of L. In some embodiments, Xaa^{T1} is an amino acid residue, and its amino group is substituted. In some embodiments, its amino group is —N(R′)—. In some embodiments, R′ is optionally substituted C₁-C₆ alkyl. In some embodiments, R′ is methyl. In some embodiments, Xaa^{T10} has no side chain. In some embodiments, Xaa^{T10} is —N(Me)-CH₂—C(O)—.

In some embodiments, Xaa^{T11} is or comprises an aromatic group. In some embodiments, the side chain of Xaa^{T11} is —CH₂—R, wherein the —CH₂— is optionally substituted, and R is as described herein. In some embodiments, R is an optionally substituted aryl or heteroaryl. In some embodiments, R is optionally substituted phenyl. In some embodiments, R is 4-hydroxyphenyl. In some embodiments, R is 4-phenylphenyl. In some embodiments, R is optionally substituted

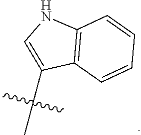 [SMILES: *c1c[nH]c2ccccc12]

In some embodiments, R is

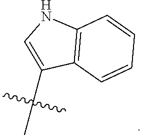 [SMILES: *c1c[nH]c2ccccc12]

In some embodiments, the side chain is that of W. In some embodiments, Xaa^{T11} is a residue of W. In some embodiments, Xaa^{T11} comprises substituted C₁-C₈ aliphatic. In some embodiments, a substitution is a polar or charged group, such as —OH, —COOH, etc. In some embodiments, a side chain is positively charged. In some embodiments, the side chain is that of R. In some embodiments, Xaa^{T11} is a residue of R.

In some embodiments, Xaa^{T6} is a residue of MeF. In some embodiments, Xaa^{T7} L. In some embodiments, Xaa^{T8} is a residue of P. In some embodiments, Xaa^{T9} is a residue of Bph. In some embodiments, Xaa^{T10} is a residue of V. In some embodiments, Xaa^{T11} is a residue of W.

In some embodiments, -(Xaa)y- is or comprises:

-(Xaa)ₐ₁-(Xaa)ₐ₂-(Xaa)ₐ₃-(Xaa)ₐ₄-(Xaa)ₐ₅-(Xaa)ₐ₆-(Xaa)ₐ₇-(Xaa)ₐ₈-(Xaa)ₐ₉-(Xaa)ₐ₁₀-(Xaa)ₐ₁₁-(Xaa)ₐ₁₂-,

wherein:

each of a1, a2, a3, a4, a5, a6, a7, a8, a9, a10, a11, and a12 is independently 0-5;

(Xaa)ₐ₄ is or comprises Xaa^{T6};

(Xaa)ₐ₆ is or comprises Xaa^{T7};

(Xaa)ₐ₇ is or comprises Xaa^{T8};

(Xaa)ₐ₈ is or comprises Xaa^{T9};

(Xaa)ₐ₉ is or comprises Xaa^{T10}; and

(Xaa)ₐ₁₀ is or comprises Xaa^{T11}

In some embodiments, a1, a2, a3, a4, a5, a6, a7, a8, a9, a10, a11, and/or a12 is independently 0. In some embodiments, a1, a2, a3, a4, a5, a6, a7, a8, a9, a10, a11, and/or a12 is independently 1. In some embodiments, a1, a2, a3, a4, a5, a6, a7, a8, a9, a10, a11, and/or a12 is independently 2. In some embodiments, a1, a2, a3, a4, a5, a6, a7, a8, a9, a10, a11, and/or a12 is independently 3. In some embodiments, a1, a2, a3, a4, a5, a6, a7, a8, a9, a10, a11, and/or a12 is independently 4. In some embodiments, a1, a2, a3, a4, a5, a6, a7, a8, a9, a10, a11, and/or a12 is independently 5. In some embodiments, a1, a2, a3, a4, a5, a6, a7, a8, a9, a10, a11, and a12 is 1.

In some embodiments, (Xaa)ₐ₁ is or comprises A. In some embodiments, a1 is 1 and (Xaa)ₐ₁ is a residue of A. In some embodiments, a1 is 0. In some embodiments, a Xaa of (Xaa)ₐ₁ (e.g., a N-terminal residue) is linked to another Xaa (e.g., of a Xaa of (Xaa)ₐ₁₃, of a C-terminal residue, etc.) as described herein. For example, in some embodiments, a N-terminal residue is linked via its amino group to a C-terminal cysteine via its —S— through a linker (e.g., —C(O)—CH₂—, wherein the —C(O)— is bonded to the amino group and the —CH₂— is bonded to the —S—).

In some embodiments, (Xaa)ₐ₂ is or comprises a Xaa^{H} residue that comprise a hydrophobic side chain. In some embodiments, a side chain is —CH₃. In some embodiments, a side chain is (CH₃)₂CHCH₂—. In some embodiments, Xaa is a residue of L. In some embodiments, Xaa is a residue of A. In some embodiments, Xaa is a residue of P. In some embodiments, (Xaa)ₐ₂ is or comprises L. In some embodiments, (Xaa)ₐ₂ is or comprises A. In some embodiments, (Xaa)ₐ₂ is or comprises P. In some embodiments, a2 is 1.

In some embodiments, the side chain of a Xaa^{H} comprises substituted C₁-C₈ aliphatic. In some embodiments, the side chain is or comprises optionally substituted C₂-C₈ aliphatic. In some embodiments, the side chain is or comprises optionally substituted C₂-C₈ alkyl. In some embodiments, the side chain is or comprises optionally substituted C₃-C₈ alkyl. In some embodiments, the side chain is or comprises optionally substituted C₄-C₈ alkyl. In some embodiments, the side chain is or comprises optionally substituted C₃-C₈ branched alkyl. In some embodiments, the side chain is or comprises optionally substituted C₄-C₈ branched alkyl. In some embodiments, the side chain is a branched C₃-C₈ alkyl. In some embodiments, the side chain is a branched C₄-C₈ alkyl. In some embodiments, the side chain is methyl. In some embodiments, the side chain is (CH₃)₂CHCH₂—. In some embodiments, Xaa^{H} is a residue of L. In some embodiments, Xaa^{H} is a residue of A.

In some embodiments, (Xaa)₃ is or comprises a residue that comprise a basic side chain (positively charged side chain). In some embodiments, the side chain of a Xaa comprises an optionally substituted aromatic basic moiety. In some embodiments, a side chain comprises optionally substituted imidazolyl. In some embodiments, the side chain of a Xaa comprises an optionally substituted non-aromatic basic moiety. In some embodiments, a side chain comprises optionally substituted guanidinyl. In some embodiments, a side chain comprises optionally substituted amino. Other types of residues can also be utilized, e.g., those comprising hydrophobic aliphatic side chains such as A. In some embodiments, a side chain is the side chain of H. In some embodiments, a side chain is the side chain of R. In some embodiments, a side chain is the side chain of A. In some embodiments, a Xaa is the residue of H. In some embodiments, a Xaa is the residue of R. In some embodiments, (Xaa)ₐ₃ is or comprises a Xaa^{H} residue as described herein. In some embodiments, a Xaa is the residue of A. In some embodiments, (Xaa)ₐ₃ is or comprises H. In some embodiments, (Xaa)ₐ₃ is or comprises R. In some embodiments, (Xaa)₃ is or comprises A. In some embodiments, a3 is 1.

In some embodiments, (Xaa)ₐ₄ is or comprises Xaa^{T6}. In some embodiments, a4 is 1. In some embodiments, (Xaa)ₐ₄ is Xaa^{T6} as described herein. In some embodiments, (Xaa)ₐ₄ is a residue of MeF or L. In some embodiments, (Xaa)ₐ₄ is or comprises MeF. In some embodiments, (Xaa)ₐ₄ is or comprises L. In some embodiments, a4 is 1.

In some embodiments, (Xaa)ₐ₅ is or comprises a Xaa^{H}. In some embodiments, (Xaa)ₐ₅ is or comprises a Xaa whose side chain is or comprises optionally substituted C₁-C₈ aliphatic. In some embodiments, a side chain is methyl. In some embodiments, Xaa is Xaa^{T10} as described herein. In some embodiments, a Xaa is a residue of V. In some embodiments, a Xaa is a residue of A. In some embodiments, a Xaa is a residue of MeG (methyl on amino group). In some embodiments, (Xaa)ₐ₅ is or comprises V. In some embodiments, (Xaa)ₐ₅ is or comprises A. In some embodiments, (Xaa)ₐ₅ is or comprises MeG. In some embodiments, a5 is 1.

In some embodiments, (Xaa)ₐ₆ is (Xaa)ₐ₂ as described herein. In some embodiments, (Xaa)ₐ₆ is or comprises a Xaa^{H} residue that comprise a hydrophobic side chain. In some embodiments, a side chain is —CH₃. In some embodiments, a side chain is (CH₃)₂CHCH₂—. In some embodiments, Xaa is a residue of L. In some embodiments, Xaa is a residue of A. In some embodiments, Xaa is a residue of P. In some embodiments, (Xaa)ₐ₆ is or comprises L. In some embodiments, (Xaa)ₐ₆ is or comprises A. In some embodiments, (Xaa)ₐ₆ is or comprises P. In some embodiments, a6 is 1.

In some embodiments, (Xaa)ₐ₆ is or comprises Xaa^{T7}. In some embodiments, a6 is 1. In some embodiments, (Xaa)ₐ₆ is Xaa^{T7} as described herein. In some embodiments, (Xaa)ₐ₆ is a residue of L or P.

In some embodiments, (Xaa)ₐ₇ is or comprises Xaa^{T8}. In some embodiments, a7 is 1. In some embodiments, (Xaa)ₐ₇ is Xaa^{T8} as described herein. In some embodiments, (Xaa)ₐ₇ is a residue of P.

In some embodiments, (Xaa)ₐ₈ is or comprises Xaa^{T9}. In some embodiments, a8 is 1. In some embodiments, (Xaa)ₐ₈ is Xaa^{T9} as described herein. In some embodiments, (Xaa)ₐ₈ is a residue of Bph. In some embodiments, (Xaa)ₐ₈ is a residue of D or S.

In some embodiments, (Xaa)ₐ₉ is or comprises Xaa^{T10} In some embodiments, a9 is 1. In some embodiments, (Xaa)ₐ₉ is Xaa^{T10} as described herein. In some embodiments, (Xaa)ₐ₉ is a residue of V, L or MeG.

In some embodiments, (Xaa)ₐ₁₀ is or comprises Xaa^{T1} In some embodiments, a10 is 1. In some embodiments, (Xaa)ₐ₁₀ is Xaa^{T11} as described herein. In some embodiments, (Xaa)ₐ₁₀ is a residue of W or R.

In some embodiments, (Xaa)ₐ₁₁ is or comprises a Xaa^{H}. In some embodiments, (Xaa)ₐ₁₁ is or comprises a Xaa whose side chain is or comprises optionally substituted C₁-C₈ aliphatic. In some embodiments, a side chain is methyl. In some embodiments, a side chain is isopropyl. In some embodiments, Xaa is Xaa^{T10} as described herein. In some embodiments, a Xaa is a residue of V. In some embodiments, a Xaa is a residue of V. In some embodiments, a Xaa is a residue of A. In some embodiments, a Xaa is a residue of MeG (methyl on amino group). In some embodiments, (Xaa)ₐ₁₁ is or comprises V. In some embodiments, (Xaa)ₐ₁₁ is or comprises A. In some embodiments, (Xaa)ₐ₁₁ is or comprises MeG. In some embodiments, a11 is 1.

In some embodiments, (Xaa)ₐ₁₂ is or comprises a residue whose side chain comprises a nucleophile. In some embodiments, (Xaa)ₐ₁₂ is or comprises a residue whose side chain comprises —S—. In some embodiments, (Xaa)ₐ₁₂ is or comprises a residue whose side chain is the side chain of C. In some embodiments, (Xaa)ₐ₁₂ is or comprises a residue of C. In some embodiments, a12 is 1. In some embodiments, a12 is greater than 1, and the last residue is a residue whose side chain comprises a nucleophile as described herein, e.g., C. In some embodiments, a Xaa of (Xaa)ₐ₁₂ (e.g., a C-terminal residue) is linked to another Xaa (e.g., of a Xaa of (Xaa)ₐ₁, of a C-terminal residue, etc.) as described herein. For example, in some embodiments, a C-terminal residue is linked via its —S— to a N-terminal cysteine via its amino group through a linker (e.g., —C(O)—CH₂—, wherein the —C(O)— is bonded to the amino group and the —CH₂— is bonded to the —S—). In some embodiments, a residue whose side chain comprises —S— (e.g., of a residue of C) is linked to an amino group of another residue through a linker (e.g., —C(O)—CH₂—, wherein the —C(O)— is bonded to the amino group and the —CH₂— is bonded to the —S—).

Exemplary sequences and data thereof include those described below:

| | | | | | | | | | | | | Frequency from |
|---|---|---|---|---|---|---|---|---|---|---|---|---|
| 1 | 2 | 3 | 4 | 5 | 6 | 7 | 8 | 9 | 10 | 11 | 12 | Screening |
| A | L | H | MeF | V | L | P | Bph | V | W | v | C | 49.4% |
| A | L | R | MeF | V | L | P | Bph | L | W | V | C | 1.8% |
| A | L | R | MeF | V | L | P | Bph | V | W | V | C | 0.7% |
| A | L | H | MeF | V | L | P | Bph | V | W | MeG | C | 0.3% |
| A | L | H | MeF | V | L | P | Bph | L | W | V | C | 0.2% |
| A | L | H | L | V | L | P | Bph | V | W | V | C | 0.2% |
| A | L | H | MeF | V | P | P | Bph | V | W | V | C | 0.2% |
| A | L | H | MeF | MeG | L | P | Bph | V | W | V | C | 0.2% |
| A | P | H | MeF | V | L | P | Bph | V | W | V | C | 0.2% |
| A | L | H | S | V | L | P | Bph | V | W | V | C | 0.1% |
| A | L | H | MeF | V | L | P | Bph | V | R | V | C | 0.1% |
| A | L | H | MeF | V | L | P | Bph | MeG | W | V | C | 0.1% |
| A | L | H | MeF | V | L | P | Bph | V | W | D | C | 0.1% |
| A | L | H | MeF | V | L | P | D | V | W | V | C | 0.1% |
| A | L | H | MeF | V | L | P | S | V | W | V | C | 0.1% |
| | | | | | | | | | | | | Binding |
| 1 | 2 | 3 | 4 | 5 | 6 | 7 | 8 | 9 | 10 | 11 | 12 | Assay* |
| A | L | H | MeF | V | L | P | Bph | V | W | V | C | 1.69 |
| A | A | H | MeF | V | L | P | Bph | V | W | V | C | 0.84 |
| A | L | A | MeF | V | L | P | Bph | V | W | V | C | 0.37 |
| A | L | H | A | V | L | P | Bph | V | W | V | C | 0.10 |
| A | L | H | MeF | A | L | P | Bph | V | W | V | C | 0.47 |
| A | L | H | MeF | V | A | P | Bph | V | W | V | C | 0.09 |
| A | L | H | MeF | V | L | A | Bph | V | W | V | C | 0.11 |
| A | L | H | MeF | V | L | P | A | V | W | V | C | 0.09 |
| A | L | H | MeF | V | L | P | Bph | A | W | V | C | 0.12 |
| A | L | H | MeF | V | L | P | Bph | V | A | V | C | 0.10 |
| A | L | H | MeF | V | L | P | Bph | V | W | A | C | 0.41 |

*Larger values represent binding. ELISA assay.

In some embodiments, a target binding moiety or

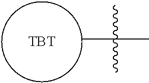

is as described above and/or as utilized in a compound in Table 1. In some embodiments,

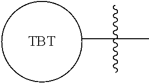

is or comprises

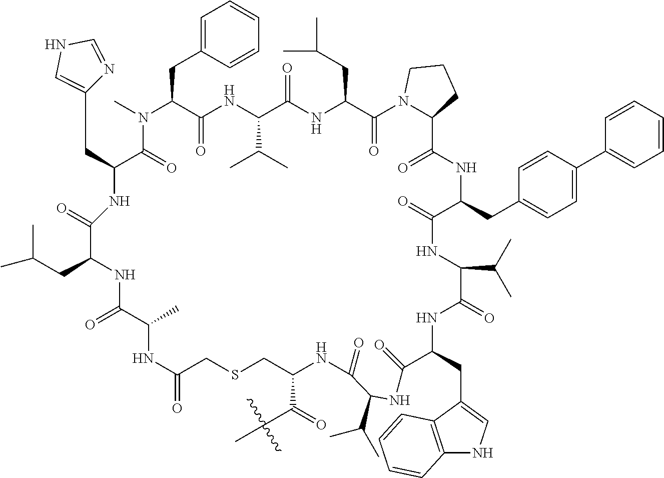 [SMILES: *C(=O)[C@@H]1CSCC(=O)N[C@@H](C)C(=O)N[C@@H](CC(C)C)C(=O)N[C@@H](Cc2c[nH]cn2)C(=O)N(C)[C@@H](Cc2ccccc2)C(=O)N[C@@H](C(C)C)C(=O)N[C@@H](CC(C)C)C(=O)N2CCC[C@H]2C(=O)N[C@@H](Cc2ccc(-c3ccccc3)cc2)C(=O)N[C@@H](C(C)C)C(=O)N[C@@H](Cc2c[nH]c3ccccc23)C(=O)N[C@@H](C(C)C)C(=O)N1]

or a salt form thereof. In some embodiments,

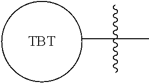

is or comprises

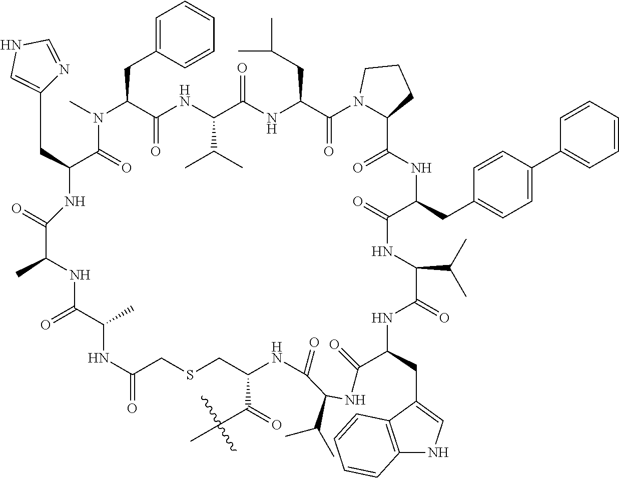 [SMILES: *C(=O)[C@@H]1CSCC(=O)N[C@@H](C)C(=O)N[C@@H](C)C(=O)N[C@@H](Cc2c[nH]cn2)C(=O)N(C)[C@@H](Cc2ccccc2)C(=O)N[C@@H](C(C)C)C(=O)N[C@@H](CC(C)C)C(=O)N2CCC[C@H]2C(=O)N[C@@H](Cc2ccc(-c3ccccc3)cc2)C(=O)N[C@@H](C(C)C)C(=O)N[C@@H](Cc2c[nH]c3ccccc23)C(=O)N[C@@H](C(C)C)C(=O)N1]

or a salt form thereof.

In some embodiments, a target binding moiety or

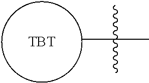

or -(Xaa)y- is or comprises a peptide that is:

(1) a polypeptide having an amino acid sequence represented by any one of SEQ ID NOS. 1-34:

| SEQ | | | | | | | | | | | | | |
|---|---|---|---|---|---|---|---|---|---|---|---|---|---|
| ID | | | | | | | | | | | | | |
| NO. | 1 | 2 | 3 | 4 | 5 | 6 | 7 | 8 | 9 | 10 | 11 | 12 | 13 |
| 1 | A | R | Ahp | Y | H | D | G | V | L | Bph | Ahp | D | C |
| 2 | A | L | H | MeF | V | L | P | Bph | V | W | V | C | |
| 3 | A | R | Ahp | Y | H | D | S | V | L | Bph | Ahp | D | C |
| 4 | A | R | Ahp | Y | H | D | Q | V | L | Bph | Ahp | D | C |
| 5 | A | R | Ahp | Y | H | D | N | V | L | Bph | Ahp | D | C |
| 6 | A | R | Ahp | Y | H | D | MetO2 | V | L | Bph | Ahp | D | C |
| 7 | A | R | Ahp | Y | H | D | G | V | T | Bph | Ahp | D | C |
| 8 | A | R | Ahp | Y | H | D | G | V | Hse | Bph | Ahp | D | C |
| 9 | A | R | Ahp | Y | H | D | G | V | MetO2 | Bph | Ahp | D | C |
| 10 | A | R | Ahp | Y | H | D | E | V | L | Bph | Ahp | D | C |
| 11 | A | R | Ahp | Y | H | D | R | V | L | Bph | Ahp | D | C |
| 12 | A | R | Ahp | Y | H | D | Har | V | L | Bph | Ahp | D | C |
| 13 | A | R | Ahp | Y | H | D | Q | V | MetO2 | Bph | Ahp | D | C |
| 14 | A | R | Ahp | Y | H | D | E | V | MetO2 | Bph | Ahp | D | C |
| 15 | A | R | Ahp | Y | H | D | R | V | MetO2 | Bph | Ahp | D | C |
| 16 | A | R | Ahp | Y | H | D | Har | V | MetO2 | Bph | Ahp | D | C |
| 17 | K | R | Ahp | Y | H | D | E | V | L | Bph | Ahp | D | C |
| | (MePEG4c) | | | | | | | | | | | | |
| 18 | A | K | Ahp | Y | H | D | E | V | L | Bph | Ahp | D | C |
| | | (MePEG4c) | | | | | | | | | | | |
| 19 | A | R | K | Y | H | D | E | V | L | Bph | Ahp | D | C |
| | | | (MePEG4c) | | | | | | | | | | |
| 20 | da | R | Ahp | Y | H | D | E | V | L | Bph | Ahp | D | C |
| 21 | A | R | Ahp | A | H | D | E | V | L | Bph | Ahp | D | C |
| 22 | A | R | Ahp | Y | W6N | D | E | V | L | Bph | Ahp | D | C |
| 23 | A | R | Ahp | Y | H | D | E | V | L | Bph | Ano | D | C |
| 24 | A | R | Ahp | Y | H | D | E | V | L | Bph | Ado | D | C |
| 25 | A | R | Ahp | Y | H | D | E | V | L | Bph | PhNle | D | C |
| 26 | A | R | Ahp | Y | H | D | E | V | L | Bph | PhNva | D | C |
| 27 | A | R | Ahp | Y | H | D | E | V | L | Bph | Ahp | A | C |
| 28 | A | Cit | Ahp | Y | H | D | E | V | L | Bph | Ahp | D | C |
| 29 | A | F3G | Ahp | Y | H | D | E | V | L | Bph | Ahp | D | C |
| 30 | A | RNMe | Ahp | Y | H | D | E | V | L | Bph | Ahp | D | C |
| 31 | A | RNNdMe | Ahp | Y | H | D | E | V | L | Bph | Ahp | D | C |
| 32 | A | RNdMe | Ahp | Y | H | D | E | V | L | Bph | Ahp | D | C |
| 33 | A | hCit | Ahp | Y | H | D | E | V | L | Bph | Ahp | D | C |
| 34 | A | 4Py2NH2 | Ahp | Y | H | D | E | V | L | Bph | Ahp | D | C |

(2) a polypeptide having an amino acid sequence represented by any one of SEQ ID NOS. 1-34 wherein the amino acid residue at the N-terminal is a chloroacetylated (e.g., at its amino group);

(3) a polypeptide having an amino acid sequence with deletions, additions, substitutions or insertion of one or more amino acids in any one of SEQ ID NOS. 1-34, which does not comprises an amino acid sequence with deletion of Cys at the C terminal in SEQ ID NOS. 1-34;

(4) a polypeptide having an amino acid sequence represented by any one of SEQ ID NOS. 1-34 with deletions, additions, substitutions or insertion of one or more amino acids in any one of SEQ ID NOS. 1-34, which does not comprises an amino acid sequence with deletion of Cys at the C terminal in any one of SEQ ID NOS. 1-34, wherein the amino acid at the N-terminal is a chloroacetylated (e.g., at its amino group); or

(5) a polypeptide in accordance with one of the above (1) to (4) wherein the polypeptide has a cyclized structure.

In some embodiments, a target binding moiety or

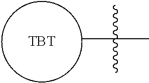

or -(Xaa)y- is or comprises a peptide that is:

(1) a polypeptide having an amino acid sequence represented by SEQ ID NO. 1 or 2:

Ala Arg Ahp Tyr His Asp Gly Val Leu Bph Ahp Asp Cys (SEQ ID NO.1),

Ala Leu His MePhe Val Leu Pro Bph Val Trp Val Cys (SEQ ID NO.2);

(2) a polypeptide having an amino acid sequence represented by SEQ ID NO. 1 or 2 wherein the Ala at the N-terminal is a chloroacetylated Ala;

(3) a polypeptide having an amino acid sequence with deletions, additions, substitutions or insertion of one or more amino acids in SEQ ID NO. 1 or 2, which does not comprises an amino acid sequence with deletion of Cys at the C terminal in SEQ ID NO. 1 or 2;

(4) a polypeptide having an amino acid sequence represented by SEQ ID NO. 1 or 2 wherein the Ala at the N-terminal is a chloroacetylated Ala with deletions, additions, substitutions or insertion of one or more amino acids in SEQ ID NO. 1 or 2, which does not comprises an amino acid sequence with deletion of Cys at the C terminal in SEQ ID NO. 1 or 2; or

(5) a polypeptide in accordance with one of the above (1) to (4) wherein the polypeptide has a cyclized structure.

In some embodiments, an amino acid residue, e.g., an amino acid residue at a N-terminus such as Ala is connected to Cys through —C(O)—CH₂—, wherein —C(O)— is boned to the amino group of Ala, and —CH₂— is bonded to —S— of Cys. In some embodiments, a N-terminal amino acid residue such as Ala is connected by reacting a chloroacetylated amino acid residue such as Ala with —SH of Cys under a suitable condition.

In some embodiments, an amino acid substitution is a conservative substitution. In some embodiments, substitutions do not significantly affect structures, properties, and/or activities of peptides and/or proteins. In some embodiments, examples of amino acid groups having side chains with similar chemical properties include 1) aliphatic side chains: glycine, alanine, valine, leucine, and isoleucine; 2) aliphatic hydroxyl side chains: serine and threonine; 3). amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: includes cysteine and methionine. In some embodiments, conservative amino acid substitutions are selected from valine-leucine-isoleucine, phenylalanine-tyrosine-tryptophan, lysine-arginine, alanine-valine, glutamic acid-aspartic acid, and asparagine-glutamine. Aforementioned amino acids can be proteinic or non-proteinic amino acids. Those skilled in the art appreciate that depending on circumstances, amino acids may be grouped based on structures, properties, activities, etc. in other ways as appropriate for the intended purpose. In some embodiments, the present disclosure provides a target binding moiety which is or comprises an amino acid sequence having deletion, substitution, insertion, and/or addition of 1 to 5 amino acids, preferably 4 or less, 3 or less, 2 or less, more preferably one amino acid or less in an amino acid sequence represented by one of SEQ IDs NO. 1 to 34 and can bind to CD38.

In some embodiments, a target binding moiety or

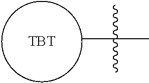

is or comprises a sequence represented by one of SEQ IDs NO. 1 to 34. In some embodiments, a target binding moiety is or comprises a cyclized structure.

In some embodiments, a target binding moiety is derived from, or

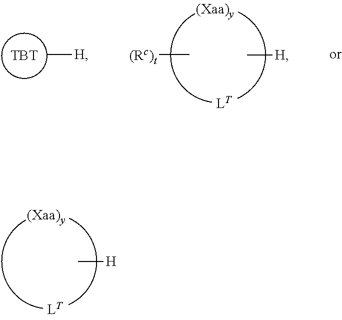

is, a structure selected from S-1 to S-32 below (with SEQ ID NO. of amino acid sequence indicated), or a pharmaceutically acceptable salt thereof:

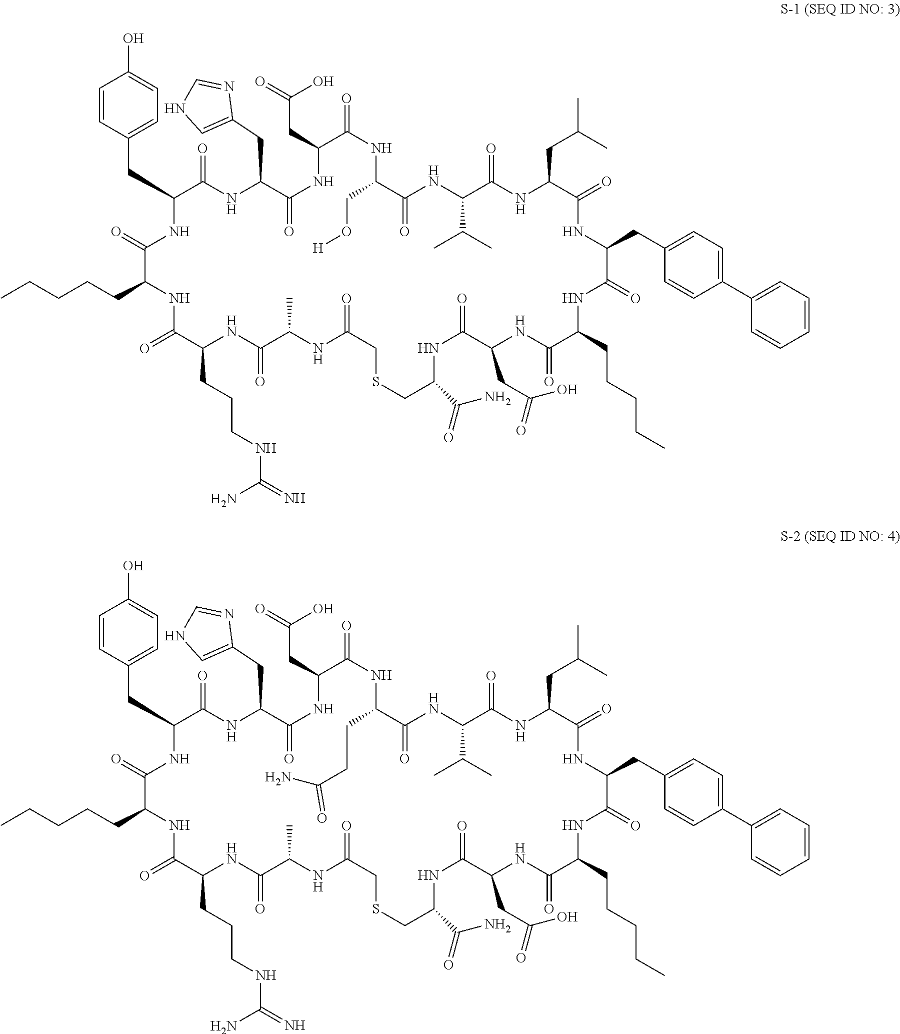

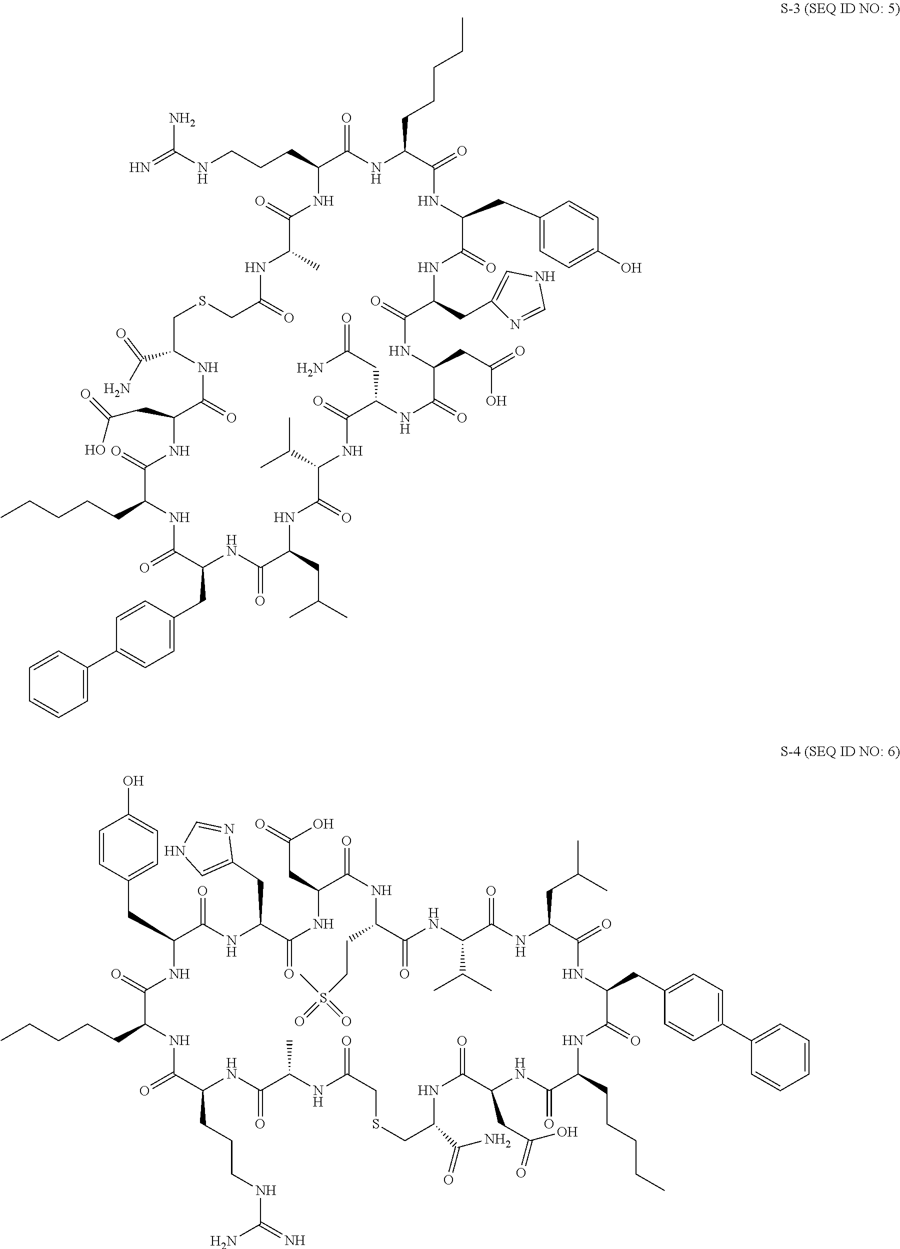

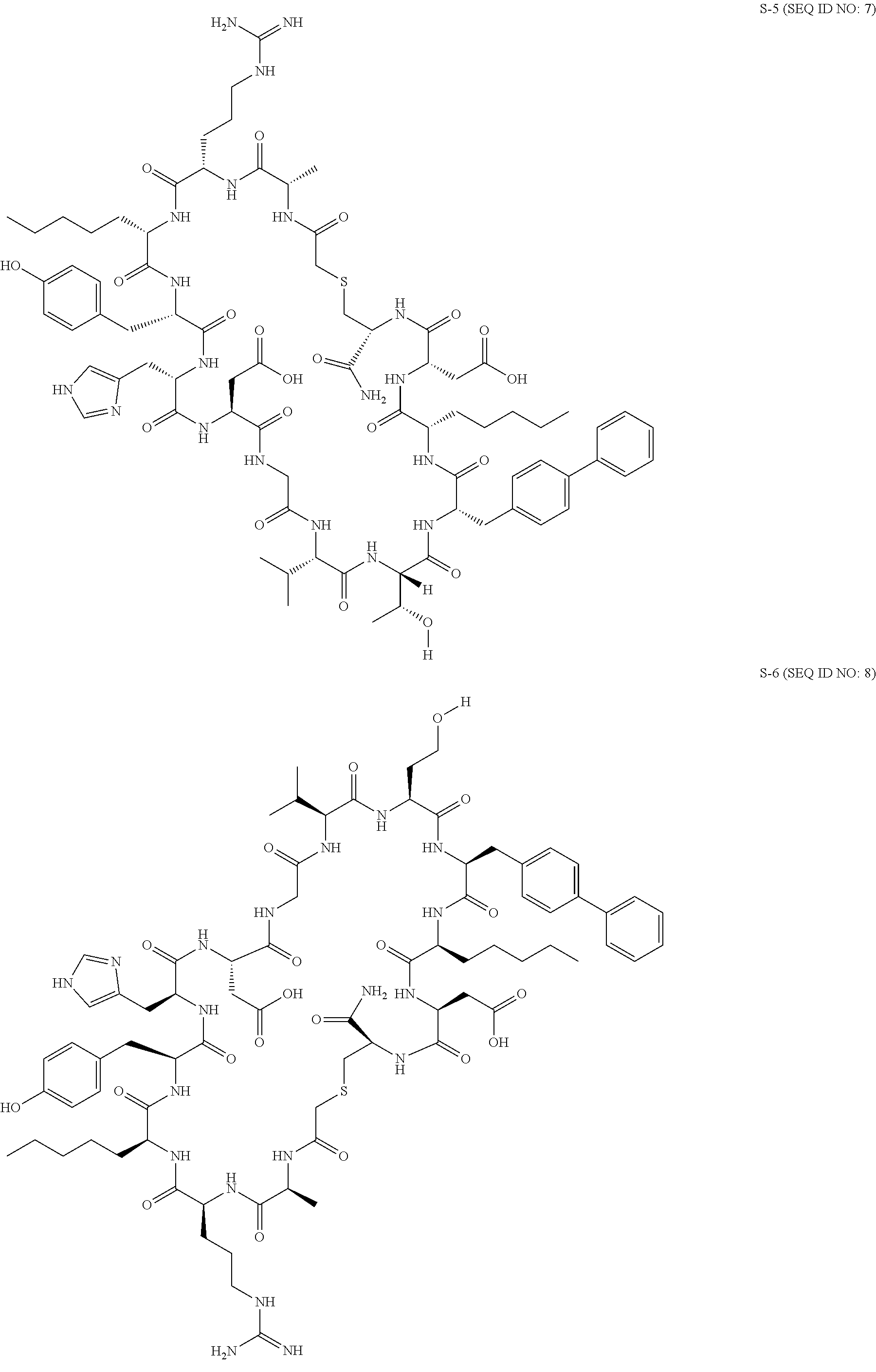

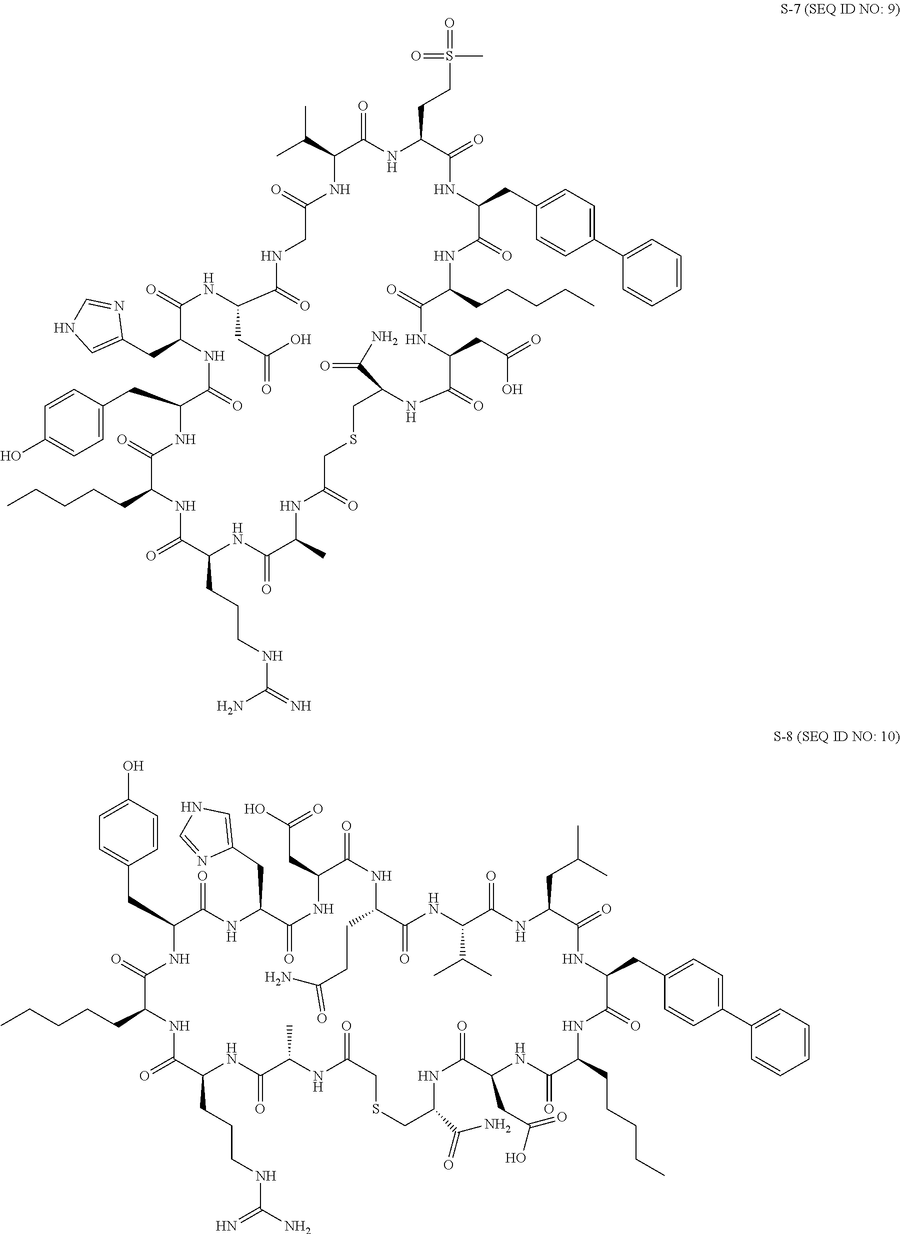

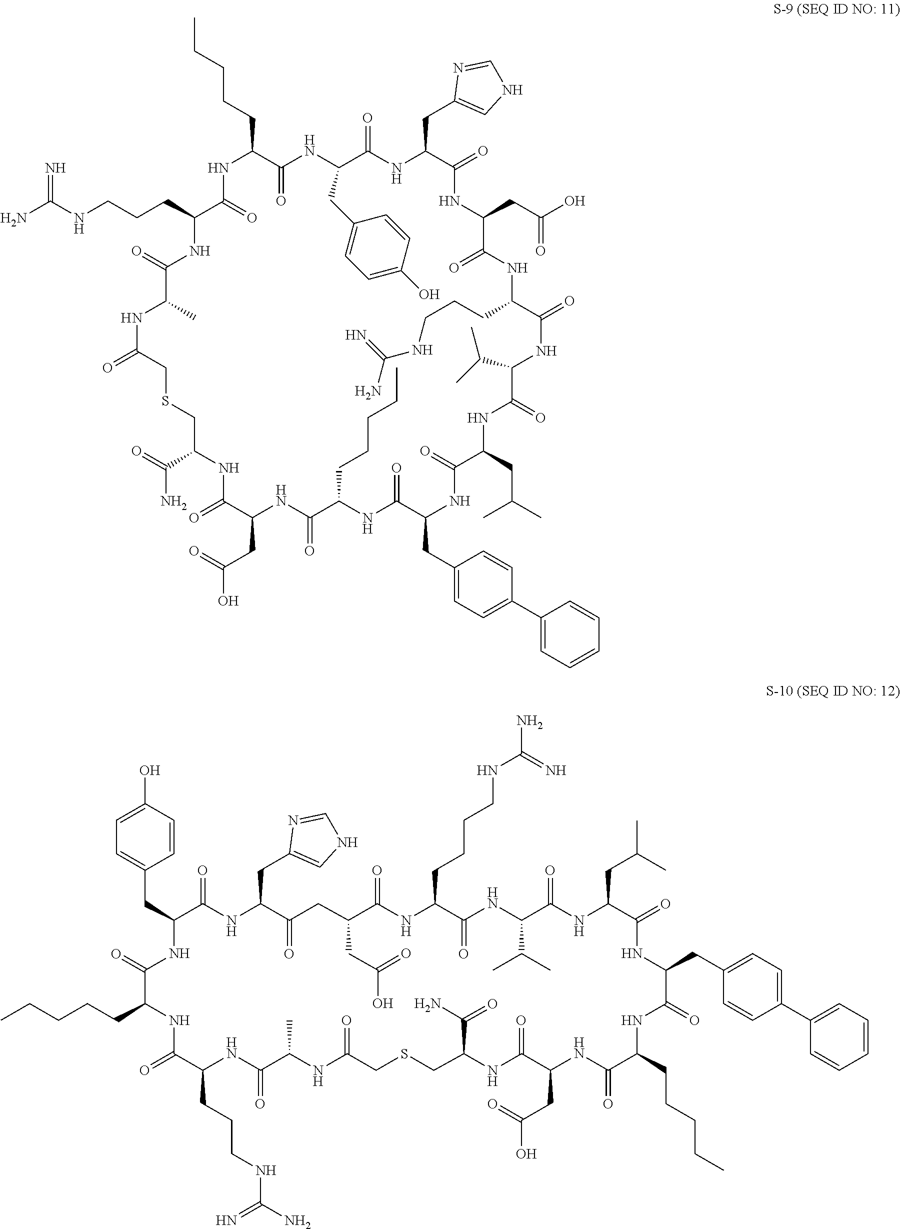

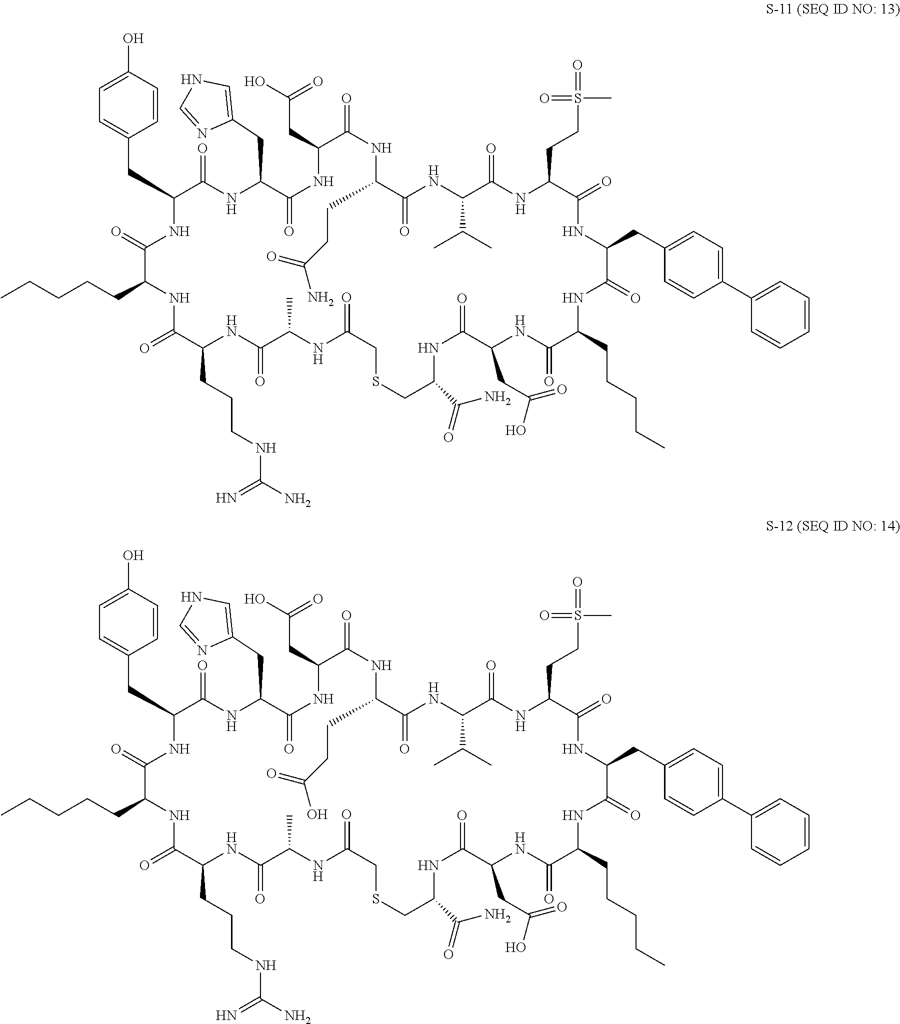

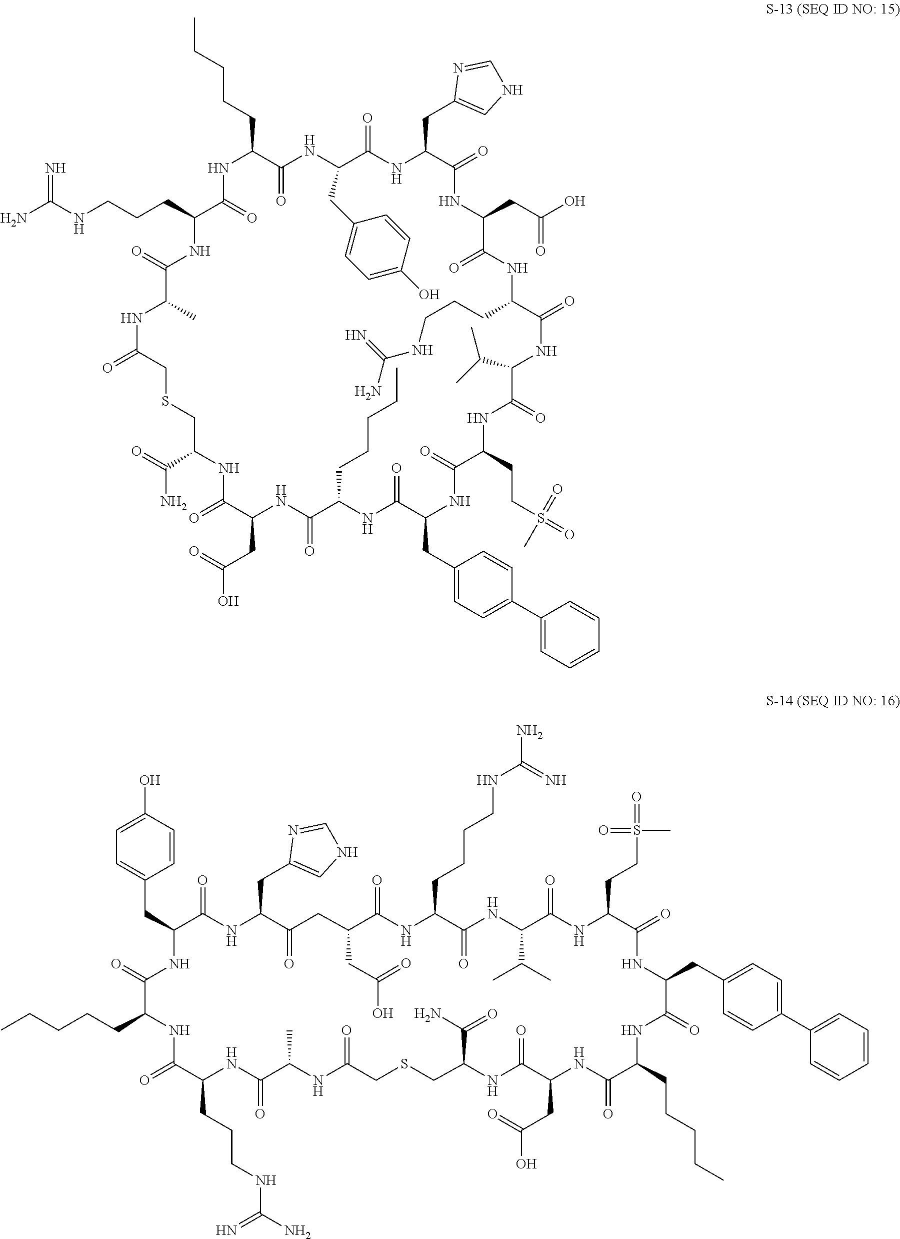

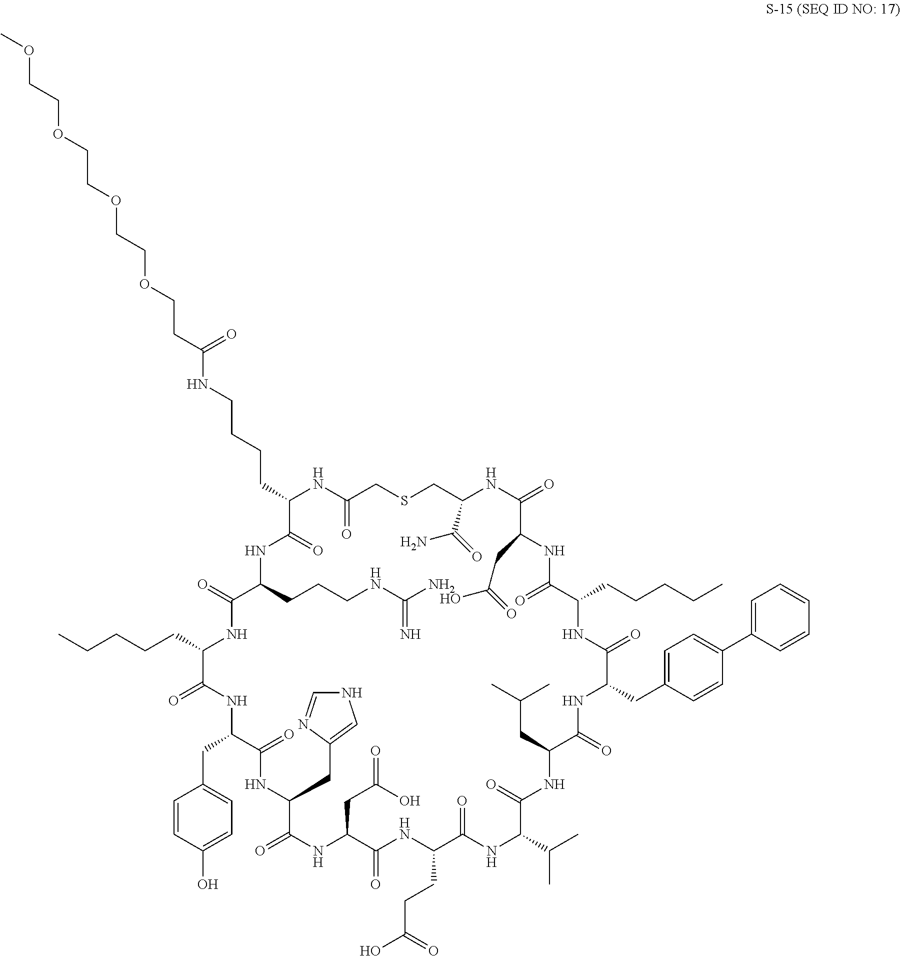 [SMILES: CCCCC[C@@H]1NC(=O)[C@H](Cc2ccc(-c3ccccc3)cc2)NC(=O)[C@H](CC(C)C)NC(=O)[C@H](C(C)C)NC(=O)[C@H](CCC(=O)O)NC(=O)[C@H](CC(=O)O)NC(=O)[C@H](Cc2c[nH]cn2)NC(=O)[C@H](Cc2ccc(O)cc2)NC(=O)[C@H](CCCCC)NC(=O)[C@H](CCCNC(=N)N)NC(=O)[C@H](CCCCNC(=O)CCOCCOCCOCCOC)NC(=O)CSC[C@@H](C(N)=O)NC(=O)[C@H](CC(=O)O)NC1=O]

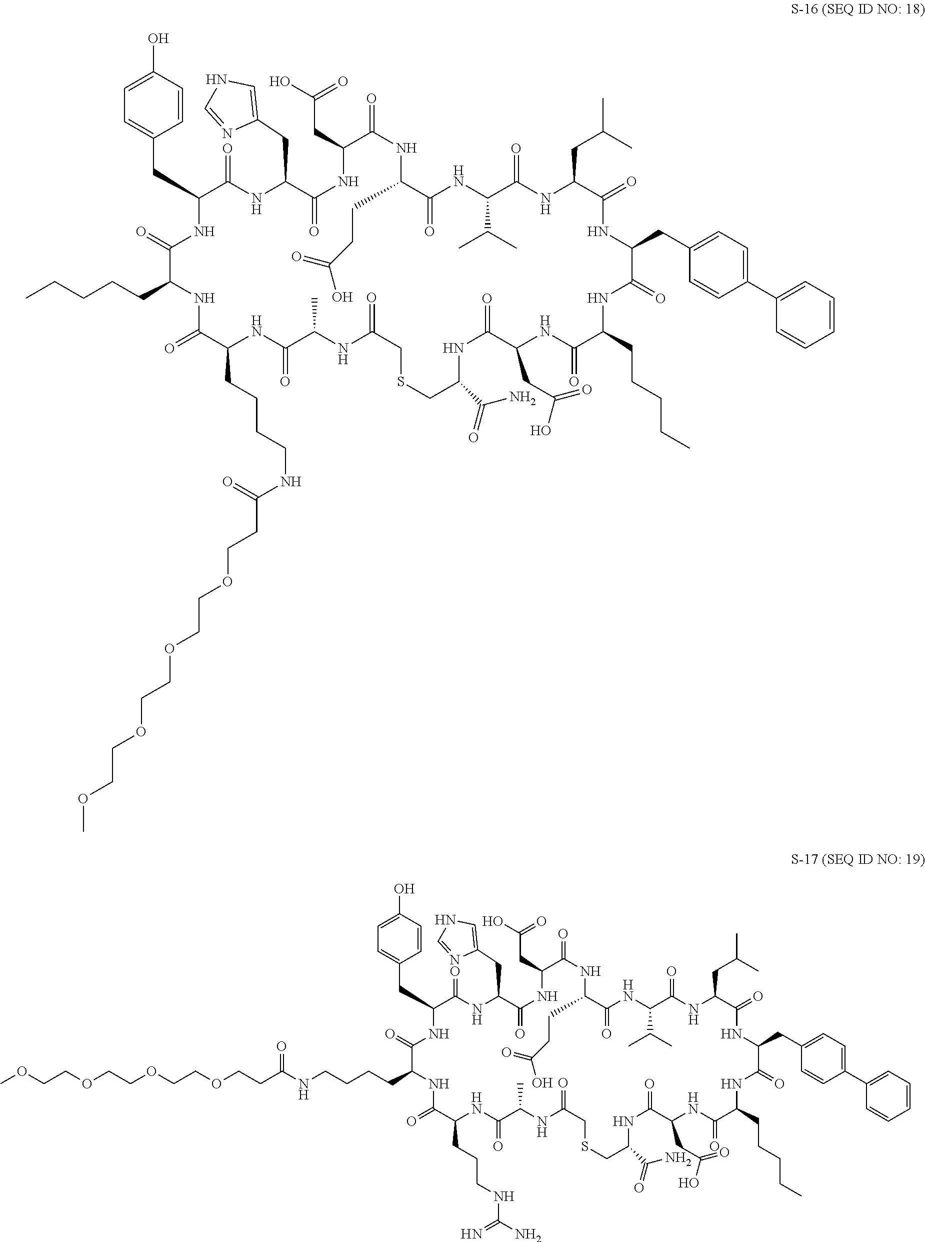

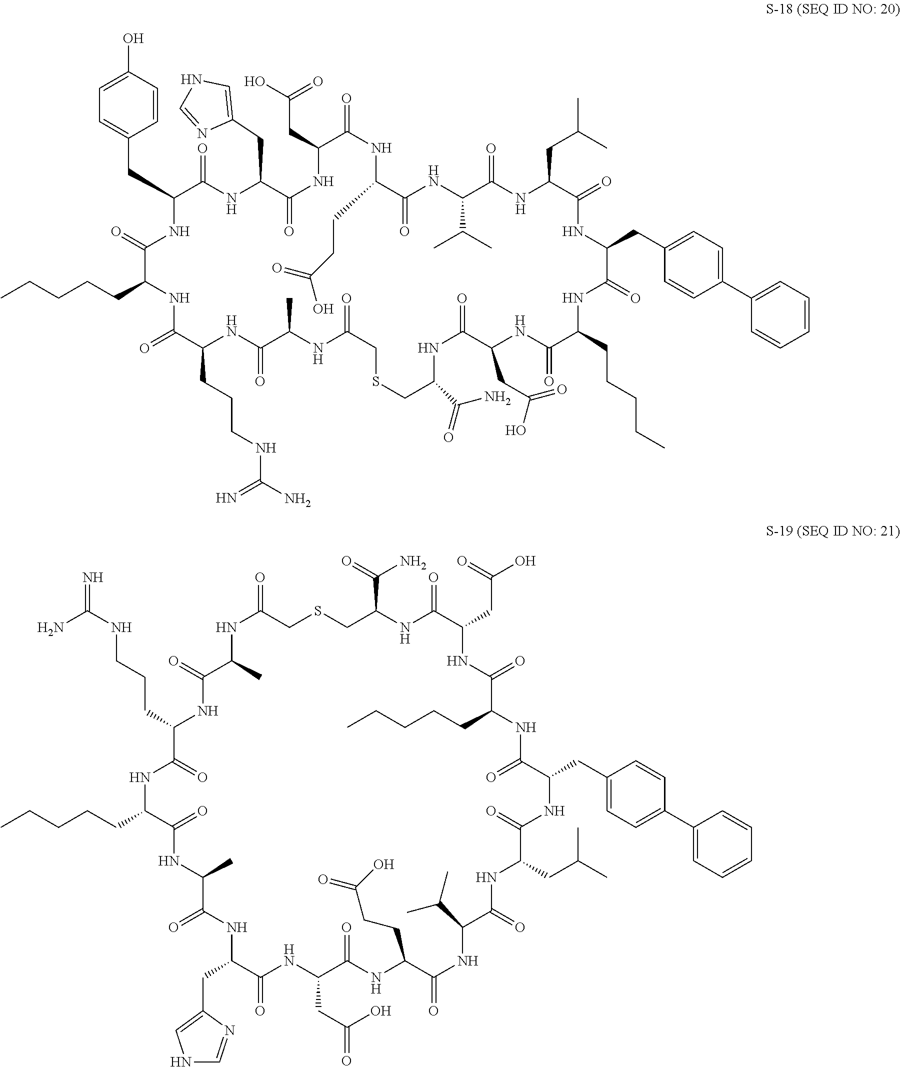

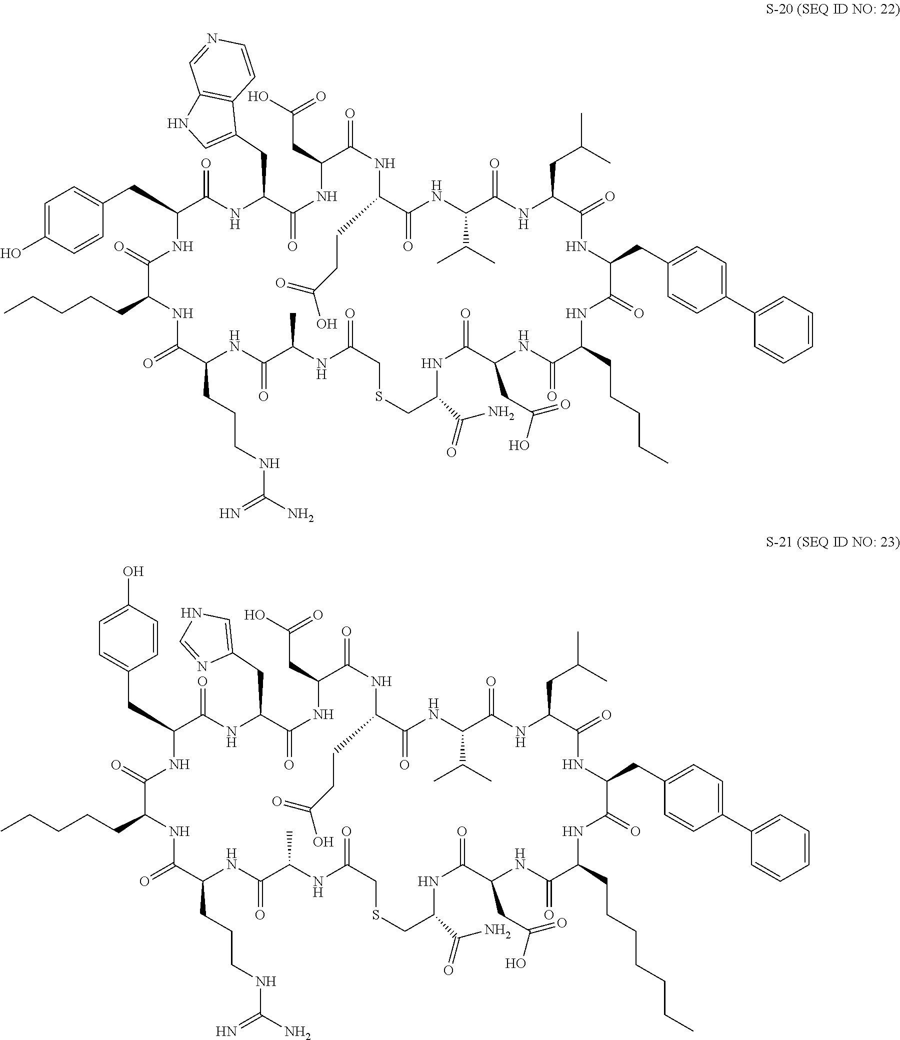

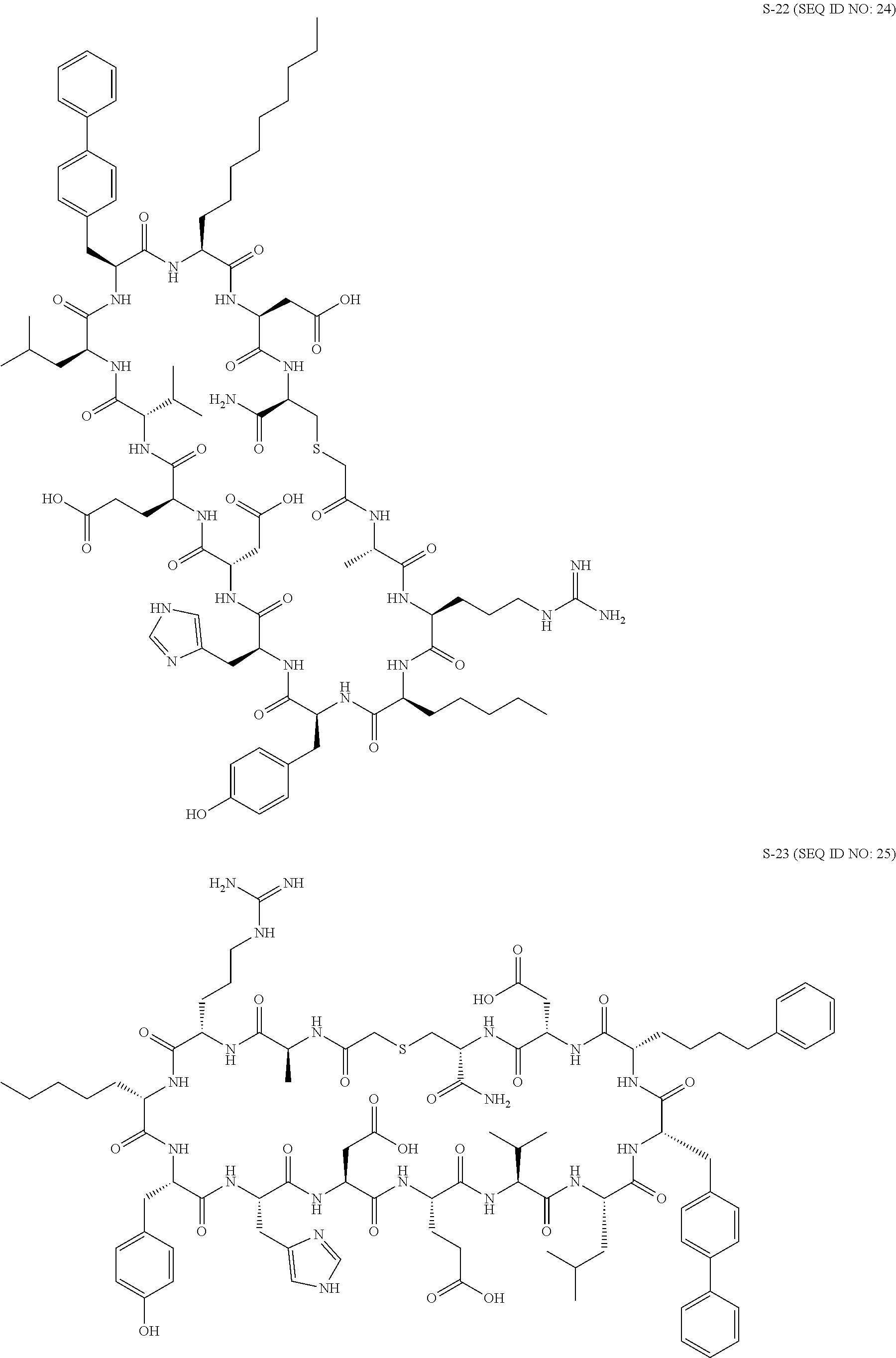

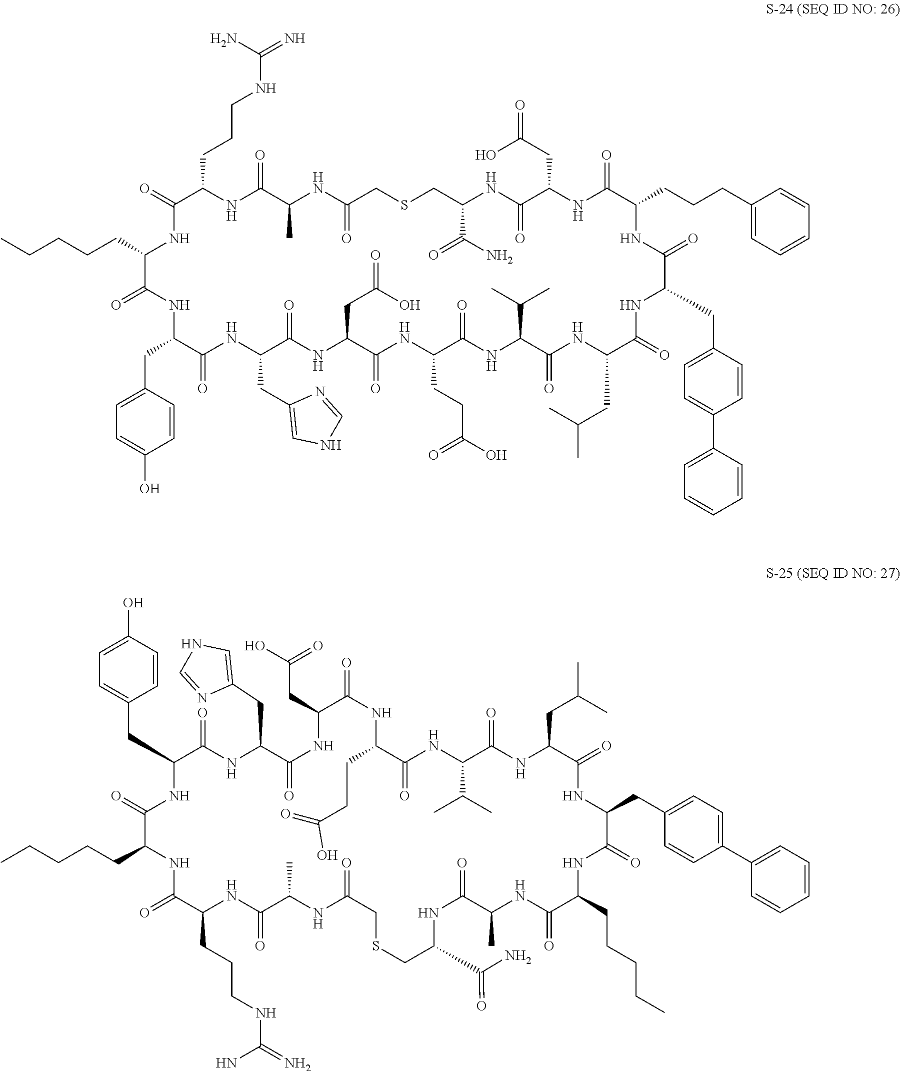

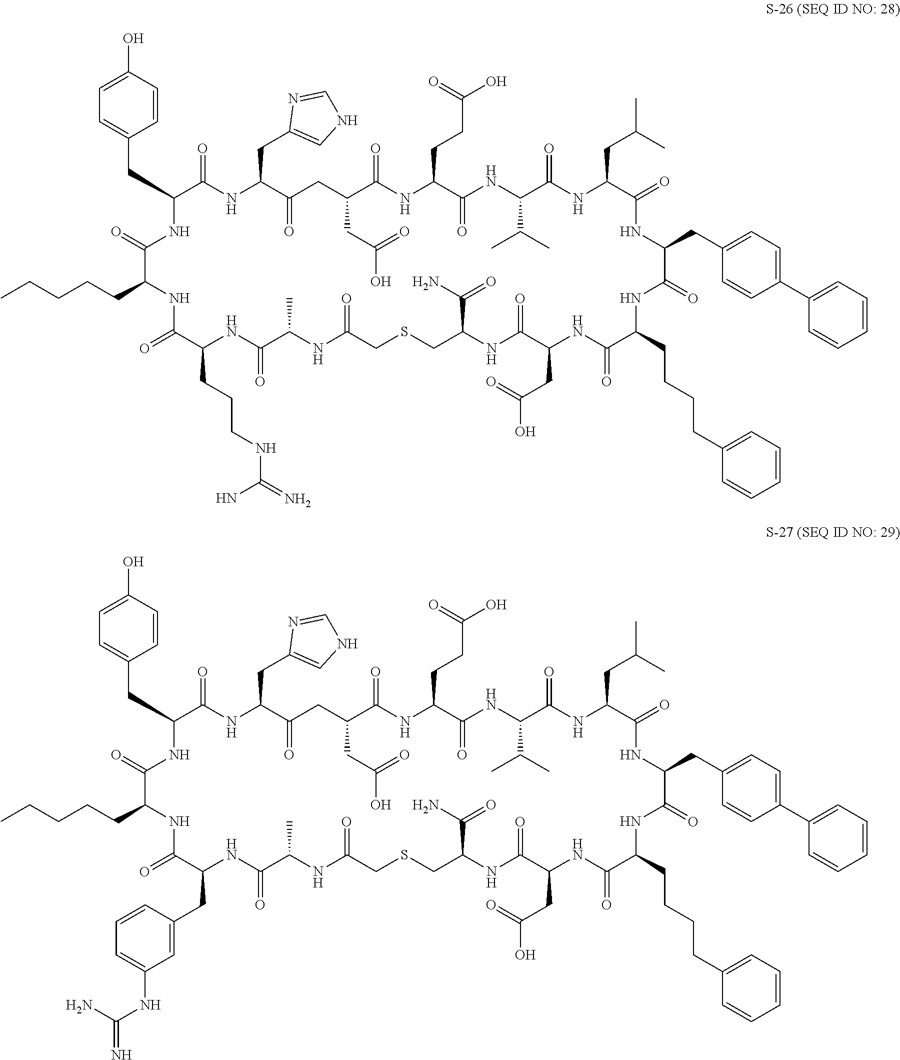

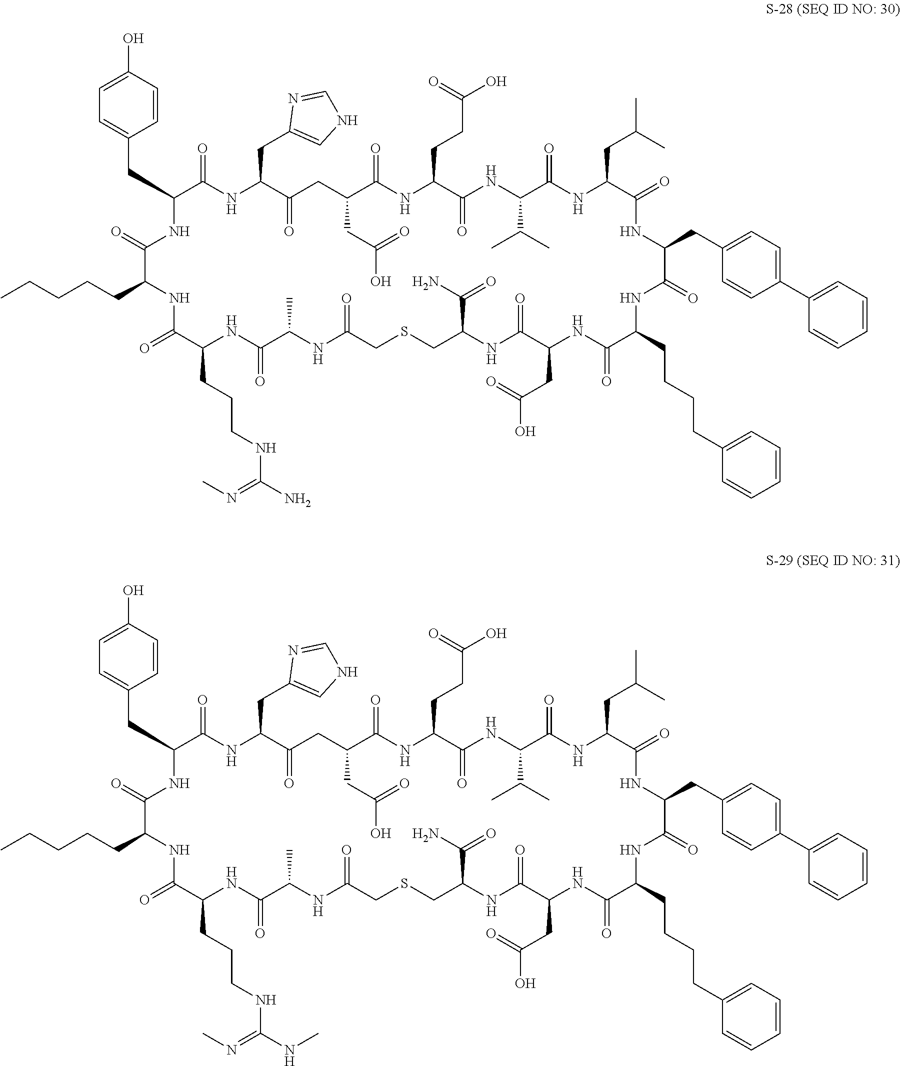

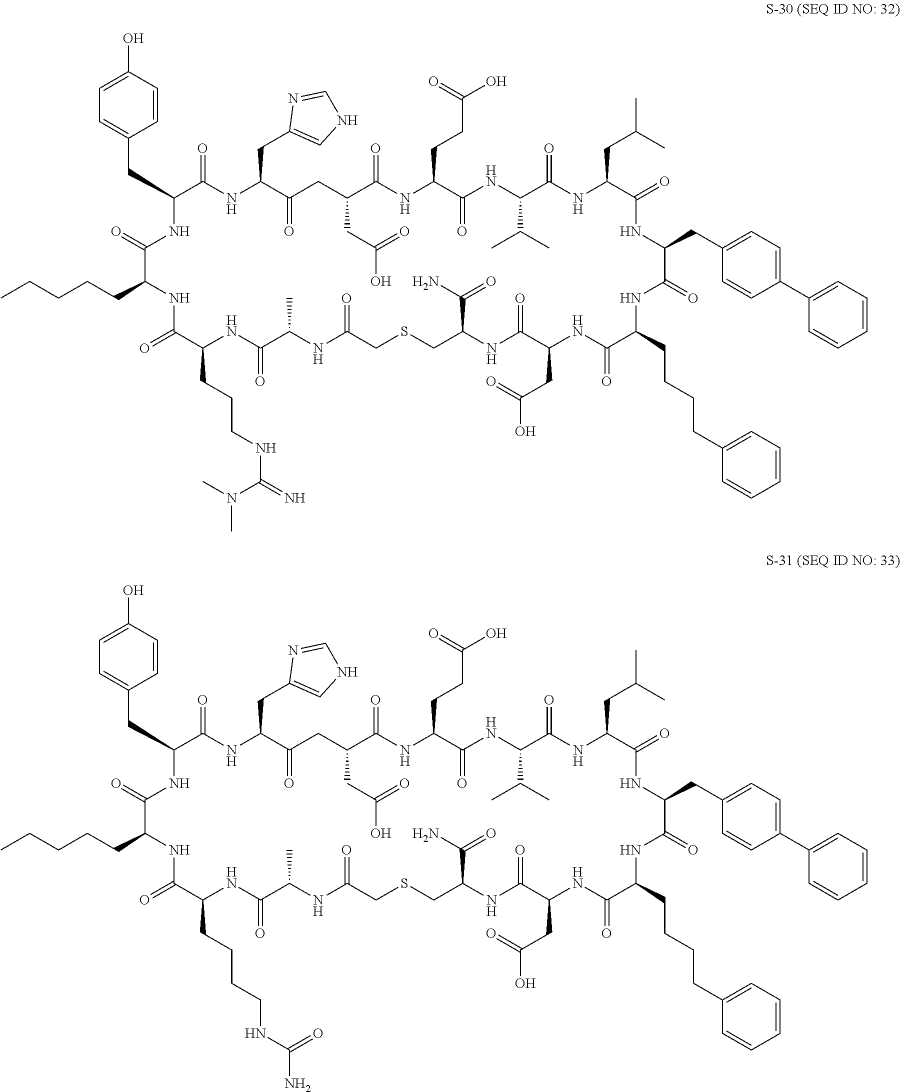

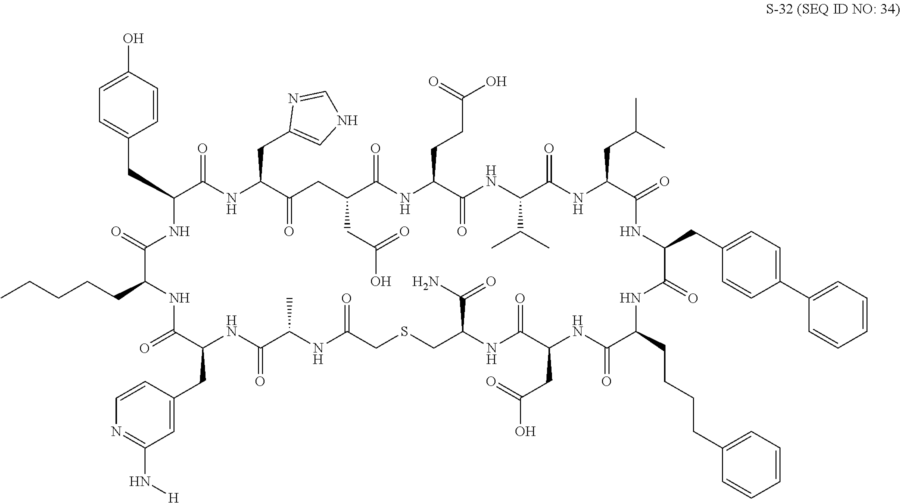 [SMILES: CCCCC[C@@H]1NC(=O)[C@H](Cc2ccnc(N)c2)NC(=O)[C@H](C)NC(=O)CSC[C@@H](C(N)=O)NC(=O)[C@H](CC(=O)O)NC(=O)[C@H](CCCCc2ccccc2)NC(=O)[C@H](Cc2ccc(-c3ccccc3)cc2)NC(=O)[C@H](CC(C)C)NC(=O)[C@H](C(C)C)NC(=O)[C@H](CCC(=O)O)NC(=O)[C@H](CC(=O)O)CC(=O)[C@H](Cc2c[nH]cn2)NC(=O)[C@H](Cc2ccc(O)cc2)NC1=O]

In some embodiments, a target binding moiety is derived from, or

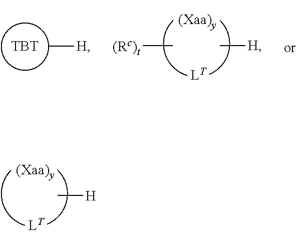

is, a structure selected from S-33 to S-39 below (with SEQ ID NO. of amino acid sequence indicated), or a pharmaceutically acceptable salt thereof:

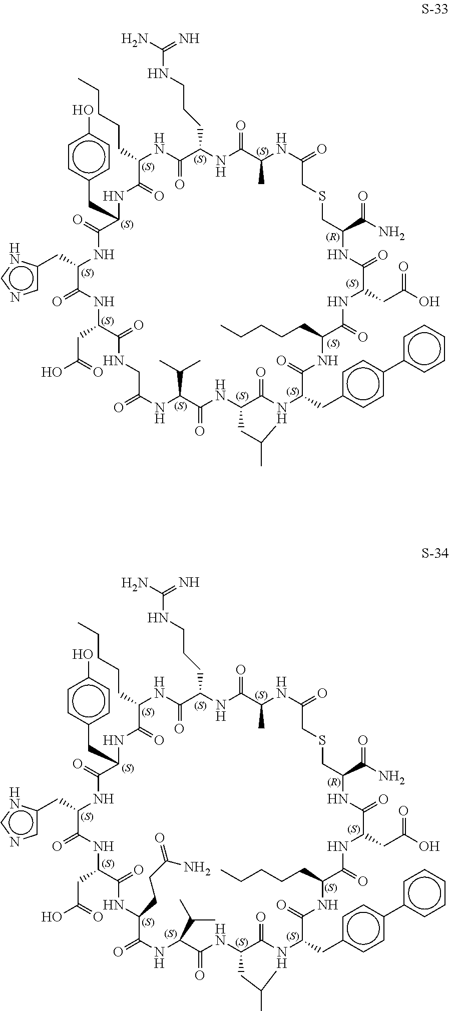

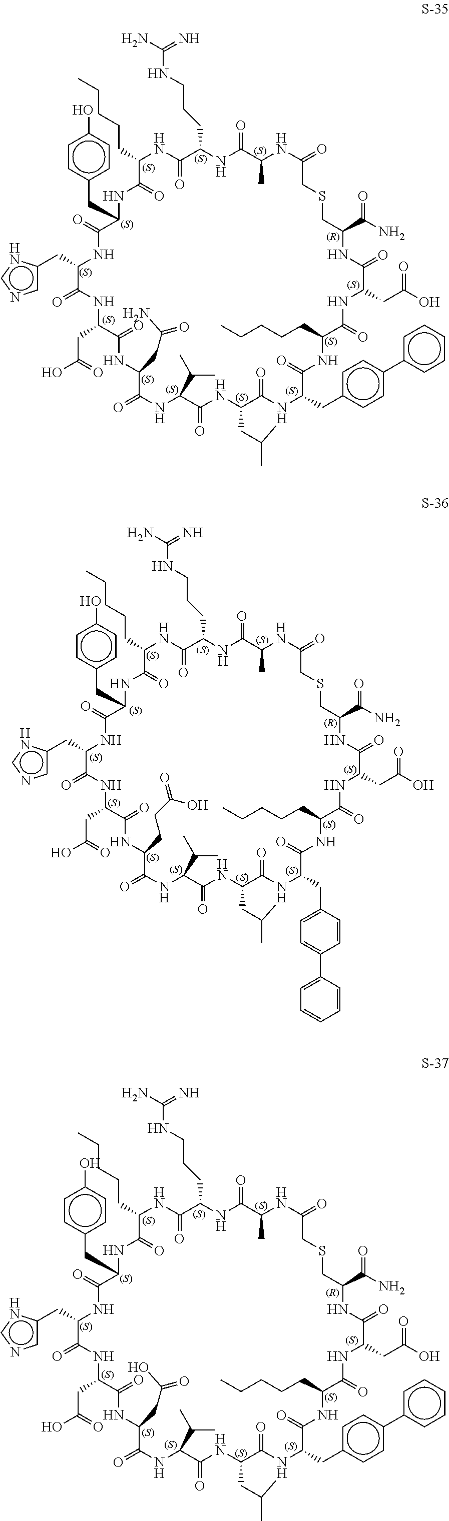

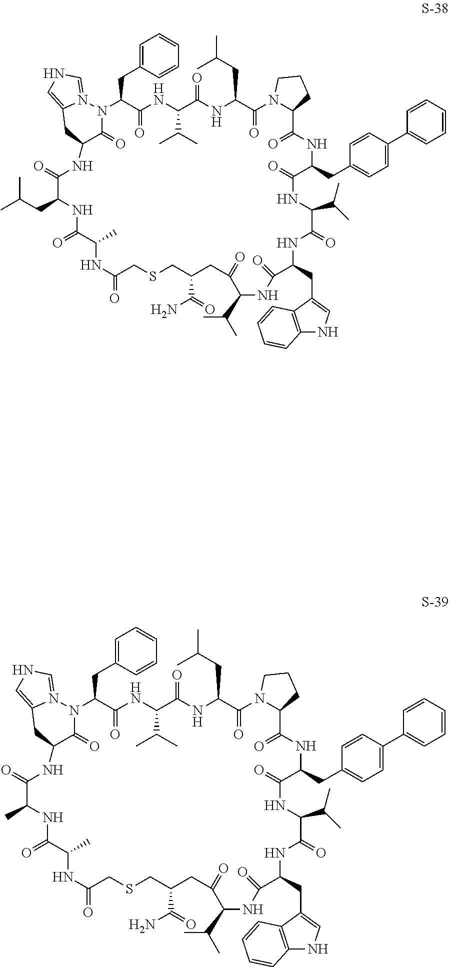

Among other things, various structures (e.g., S-1 to S-39) were assessed in various assays and were shown to bind to CD38.

As those skilled in the art will appreciate, structures (e.g., S-1 to S-39) may be connected to the rest of a molecule (e.g., an antibody binding moiety optionally through a linker) via a number of suitable ways in accordance with the present disclosure (e.g., through side chains, such as amino groups of certain side chains, N-terminus, C-terminus, etc.)

In some embodiments, a peptide unit, e.g., a target binding moiety having the structure of

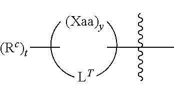

or a salt thereof, comprises a functional group in an amino acid residue that can react with a functional group of another amino acid residue. In some embodiments, a peptide unit comprises an amino acid residue with a side chain which comprises a functional group that can react with another functional group of the side chain of another amino acid residue to form a linkage (e.g., see moieties in Table A-1, Table 1, etc.). In some embodiments, one functional group of one amino acid residue is connected to a functional group of another amino acid residue to form a linkage (or bridge). Linkages are bonded to backbone atoms of peptide units and comprise no backbone atoms. In some embodiments, a peptide unit comprises a linkage formed by two side chains of non-neighboring amino acid residues. In some embodiments, a linkage is bonded to two backbone atoms of two non-neighboring amino acid residues. In some embodiments, both backbone atoms bonded to a linkage are carbon atoms. In some embodiments, a linkage has the structure of L^{b}, wherein L^{b} is L^{a} as described in the present disclosure, wherein L^{a} is not a covalent bond. In some embodiments, L^{a} comprises -Cy-. In some embodiments, L^{a} comprises -Cy-, wherein -Cy- is optionally substituted heteroaryl. In some embodiments,

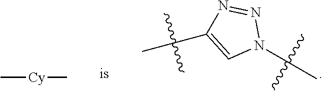

In some embodiments, L^{a} is

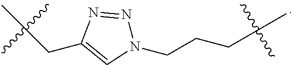 [SMILES: *CCCn1cc(C*)nn1]

In some embodiments, such an L^{a} can be formed by a —N₃ group of the side chain of one amino acid residue, and the —≡— of the side chain of another amino acid residue. In some embodiments, a linkage is formed through connection of two thiol groups, e.g., of two cysteine residues. In some embodiments, L^{a} comprises —S—S—. In some embodiments, L^{a} is —CH₂—S—S—CH₂—. In some embodiments, a linkage is formed through connection of an amino group (e.g., —NH₂ in the side chain of a lysine residue) and a carboxylic acid group (e.g., —COOH in the side chain of an aspartic acid or glutamic acid residue). In some embodiments, L^{a} comprises —C(O)—N(R′)—. In some embodiments, L^{a} comprise —C(O)—NH—. In some embodiments, L^{a} is —CH₂CONH—(CH₂)₃—. In some embodiments, L^{a} comprises —C(O)—N(R′)—, wherein R′ is R, and is taken together with an R group on the peptide backbone to form a ring (e.g., in A-34). In some embodiments, L^{a} is —(CH₂)₂—N(R′)—CO—(CH₂)₂—. In some embodiments, -Cy- is optionally substituted phenylene. In some embodiments, -Cy- is optionally substituted 1,2-phenylene. In some embodiments, L^{a} is

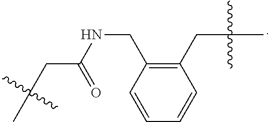 [SMILES: *CC(=O)NCc1ccccc1C*]

n some embodiments, L^{a} is

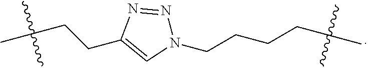 [SMILES: *CCCCn1cc(CC*)nn1]

In some embodiments, L^{a} is optionally substituted bivalent C₂₋₂₀ bivalent aliphatic. In some embodiments, L^{a} is optionally substituted —(CH₂)₉—CH═CH—(CH₂)₉—. In some embodiments, L^{a} is —(CH₂)₃—CH═CH—(CH₂)₃—.

In some embodiments, two amino acid residues bonded to a linkage are separated by 1, 2, 3, 4, 5, 6, 7, 8, 9, 10, 11, 12, 13, 14, 15, or more than 15 amino acid residues between them (excluding the two amino acid residues bonded to the linkage). In some embodiments, the number is 1. In some embodiments, the number is 2. In some embodiments, the number is 3. In some embodiments, the number is 4. In some embodiments, the number is 5. In some embodiments, the number is 6. In some embodiments, the number is 7. In some embodiments, the number is 8. In some embodiments, the number is 9. In some embodiments, the number is 10. In some embodiments, the number is 11. In some embodiments, the number is 12. In some embodiments, the number is 13. In some embodiments, the number is 14. In some embodiments, the number is 15.

In some embodiments, a target binding moiety comprises a peptide unit, and an antibody binding moiety is connected to a backbone atom of the peptide unit optionally via a linker. In some embodiments, a target binding moiety comprises a peptide unit, and an antibody binding moiety is connected to an atom of a side chain, e.g., through an atom or group in the side chain, of an amino acid residue of the peptide unit optionally via a linker. For example, in some embodiments, an antibody binding moiety is connected through a —SH, —OH, —COOH, or —NH₂ of a side chain.

### Amino Acids

In some embodiments, provided compounds and agents may comprise one or more amino acid moieties, e.g., in universal antibody binding moieties, linker moieties, etc. Amino acid moieties can either be those of natural amino acids or unnatural amino acids. In some embodiments, an amino acid has the structure of formula A-I:

NH(R^{a1})-L^{a1}-C(R^{a2})(R^{a3})-L^{a2}-COOH, A-I

or a salt thereof, wherein each variable is independent as described in the present disclosure. In some embodiments, an amino acid residue, e.g., of an amino acid having the structure of formula A-I, has the structure of —N(R^{a1})-L^{a1}-C(R^{a2})(R^{a3})-L^{a2}-CO—. In some embodiments, each amino acid residue in a peptide independently has the structure of —N(R^{a1})-L^{a1}-C(R^{a2})(R^{a3})-L^{a2}-CO—.

In some embodiments, L^{a1} is a covalent bond. In some embodiments, a compound of formula A-I is of the structure NH(R^{a1})—C(R^{a2})(R^{a3})-L^{a2}-COOH. In some embodiments, L^{a2} is —CH₂SCH₂—.

In some embodiments, L^{a2} is a covalent bond. In some embodiments, a compound of formula A-I is of the structure NH(R^{a1})-L^{a1}-C(R^{a2})(R^{a3})—COOH. In some embodiments, an amino acid residue has the structure of —N(R^{a1})-L^{a1}-C(R^{a2})(R^{a3})—CO—. In some embodiments, L^{a1} is —CH₂CH₂S—. In some embodiments, L^{a1} is —CH₂CH₂S—, wherein the CH₂ is bonded to NH(R^{a1})

In some embodiments, L^{a1} is a covalent bond and L^{a2} is a covalent bond. In some embodiments, a compound of formula A-I is of the structure NH(R^{a1})—C(R^{a2})(R^{a3})—COOH. In some embodiments, a compound of formula A-I is of the structure NH(R^{a1})—CH(R^{a2})—COOH. In some embodiments, a compound of formula A-I is of the structure NH(R^{a1})—CH(R^{a3})—COOH. In some embodiments, a compound of formula A-I is of the structure NH₂—CH(R^{a2})—COOH. In some embodiments, a compound of formula A-I is of the structure NH₂—CH(R^{a3})—COOH. In some embodiments, an amino acid residue has the structure of —N(R^{a1})—C(R^{a2})(R^{a3})—CO—. In some embodiments, an amino acid residue has the structure of —N(R^{a1})—CH(R^{a2})—CO—. In some embodiments, an amino acid residue has the structure of —N(R^{a1})—CH(R^{a3})—CO—. In some embodiments, an amino acid residue has the structure of —NH—CH(R^{a2})—CO—. In some embodiments, an amino acid residue has the structure of —NH—CH(R^{a3})—CO—.

In some embodiments, L^{a} is a covalent bond. In some embodiments, L^{a} is optionally substituted C₁₋₆ bivalent aliphatic. In some embodiments, L^{a} is optionally substituted C₁₋₆ alkylene. In some embodiments, L^{a} is —CH₂—. In some embodiments, L^{a} is —CH₂CH₂—. In some embodiments, L^{a} is —CH₂CH₂CH₂—.

In some embodiments, R′ is R. In some embodiments, R^{a1} is R, wherein R is as described in the present disclosure. In some embodiments, R^{a1} is R, wherein R methyl. In some embodiments, R^{a2} is R, wherein R is as described in the present disclosure. In some embodiments, R^{a3} is R, wherein R is as described in the present disclosure. In some embodiments, each of R^{a1}, R^{a2}, and R^{a3} is independently R, wherein R is as described in the present disclosure.

In some embodiments, R^{a1} is hydrogen. In some embodiments, R^{a2} is hydrogen. In some embodiments, R^{a3} is hydrogen. In some embodiments, R^{a1} is hydrogen, and at least one of R^{a2} and R^{a3} is hydrogen. In some embodiments, R^{a1} is hydrogen, one of R^{a2} and R^{a3} is hydrogen, and the other is not hydrogen. In some embodiments, R^{a2} is -L^{a}-R and R^{a3} is —H. In some embodiments, R^{a3} is -L^{a}-R and R^{a2} is —H. In some embodiments, R^{a2} is —CH₂—R and R^{a3} is —H. In some embodiments, R^{a3} is —CH₂—R and R^{a2} is —H. In some embodiments, R^{a2} is R and R^{a3} is —H. In some embodiments, R^{a3} is R and R^{a2} is —H.

In some embodiments, R^{a2} is -L^{a}-R, wherein R is as described in the present disclosure. In some embodiments, R^{a2} is -L^{a}-R, wherein R is an optionally substituted group selected from C₃₋₃₀ cycloaliphatic, C₅₋₃₀ aryl, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, R^{a2} is -L^{a}-R, wherein R is an optionally substituted group selected from C₆₋₃₀ aryl and 5-30 membered heteroaryl having 1-10 heteroatoms independently selected from oxygen, nitrogen, sulfur, phosphorus and silicon. In some embodiments, R^{a2} is a side chain of an amino acid. In some embodiments, R^{a2} is a side chain of a standard amino acid.

In some embodiments, R^{a3} is -L^{a}-R, wherein R is as described in the present disclosure. In some embodiments, R^{a3} is -L^{a}-R, wherein R is an optionally substituted group selected from C₃₋₃₀ cycloaliphatic, C₅₋₃₀ aryl, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, R^{a3} is -L^{a}-R, wherein R is an optionally substituted group selected from C₆₋₃₀ aryl and 5-30 membered heteroaryl having 1-10 heteroatoms independently selected from oxygen, nitrogen, sulfur, phosphorus and silicon. In some embodiments, R^{a3} is a side chain of an amino acid. In some embodiments, R^{a3} is a side chain of a standard amino acid.

In some embodiments, R is an optionally substituted C₁₋₆ aliphatic. In some embodiments, R is an optionally substituted C₁₋₆ alkyl. In some embodiments, R is —CH₃. In some embodiments, R is optionally substituted pentyl. In some embodiments, R is n-pentyl.

In some embodiments, R is a cyclic group. In some embodiments, R is an optionally substituted C₃₋₃₀ cycloaliphatic group. In some embodiments, R is cyclopropyl.

In some embodiments, R is an optionally substituted aromatic group, and an amino acid residue of an amino acid of formula A-I is a Xaa^{A}. In some embodiments, R^{a2} or R^{a3} is —CH₂—R, wherein R is an optionally substituted aryl or heteroaryl group. In some embodiments, R is optionally substituted phenyl. In some embodiments, R is phenyl. In some embodiments, R is optionally substituted phenyl. In some embodiments, R is 4-trifluoromethylphenyl. In some embodiments, R is 4-phenylphenyl. In some embodiments, R is optionally substituted 5-30 membered heteroaryl having 1-10 heteroatoms independently selected from oxygen, nitrogen, sulfur, phosphorus and silicon. In some embodiments, R is optionally substituted 5-14 membered heteroaryl having 1-5 heteroatoms independently selected from oxygen, nitrogen, and sulfur. In some embodiments, R is

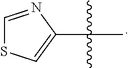 [SMILES: *c1cscn1]

In some embodiments, R is optionally substituted pyridinyl. In some embodiments, R is 1- pyridinyl. In some embodiments, R is 2-pyridinyl. In some embodiments, R is 3-pyridinyl. In some embodiments, R is

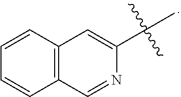 [SMILES: *c1cc2ccccc2cn1]

In some embodiments, R′ is-COOH. In some embodiments, a compound of and an amino acid residue of an amino acid of formula A-I is a Xaa^{N}.

In some embodiments, R′ is-NH₂. In some embodiments, a compound of an amino acid residue of an amino acid of formula A-I is a Xaa^{P}.

In some embodiments, R^{a2} or R^{a3} is R, wherein R is C₁₋₂₀ aliphatic as described in the present disclosure. In some embodiments, a compound of an amino acid residue of an amino acid of formula A-I is a Xaa^{H}. In some embodiments, R is —CH₃. In some embodiments, R is ethyl. In some embodiments, R is propyl. In some embodiments, R is n-propyl. In some embodiments, R is butyl. In some embodiments, R is n-butyl. In some embodiments, R is pentyl. In some embodiments, R is n-pentyl. In some embodiments, R is cyclopropyl.

In some embodiments, two or more of R^{a1}, R^{a2}, and R^{a3} are R and are taken together to form an optionally substituted ring as described in the present disclosure.

In some embodiments, R^{a1} and one of R^{a2} and R^{a3} are R and are taken together to form an optionally substituted 3-6 membered ring having no additional ring heteroatom other than the nitrogen atom to which R^{a1} is bonded to. In some embodiments, a formed ring is a 5-membered ring as in proline.

In some embodiments, R^{a2} and R^{a3} are R and are taken together to form an optionally substituted 3-6 membered ring as described in the present disclosure. In some embodiments, R^{a2} and R^{a3} are R and are taken together to form an optionally substituted 3-6 membered ring having one or more nitrogen ring atom. In some embodiments, R^{a2} and R^{a3} are R and are taken together to form an optionally substituted 3-6 membered ring having one and no more than one ring heteroatom which is a nitrogen atom. In some embodiments, a ring is a saturated ring.

In some embodiments, an amino acid is a natural amino acid. In some embodiments, an amino acid is an unnatural amino acid. In some embodiments, an amino acid is an alpha-amino acid. In some embodiments, an amino acid is a beta-amino acid. In some embodiments, a compound of formula A-I is a natural amino acid. In some embodiments, a compound of formula A-I is an unnatural amino acid.

In some embodiments, an amino acid comprises a hydrophobic side chain. In some embodiments, an amino acid with a hydrophobic side chain is A, V, I, L, M, F, Y or W. In some embodiments, an amino acid with a hydrophobic side chain is A, V, I, L, M, or F. In some embodiments, an amino acid with a hydrophobic side chain is A, V, I, L, or M. In some embodiments, an amino acid with a hydrophobic side chain is A, V, I, or L. In some embodiments, a hydrophobic side chain is R wherein R is C₁₋₁₀ aliphatic. In some embodiments, R is C₁₋₁₀ alkyl. In some embodiments, R is methyl. In some embodiments, R is ethyl. In some embodiments, R is propyl. In some embodiments, R is butyl. In some embodiments, R is pentyl. In some embodiments, R is n-pentyl. In some embodiments, an amino acid with a hydrophobic side chain is NH₂CH(CH₂CH₂CH₂CH₂CH₃)COOH. In some embodiments, an amino acid with a hydrophobic side chain is (S)—NH₂CH(CH₂CH₂CH₂CH₂CH₃)COOH. In some embodiments, an amino acid with a hydrophobic side chain is (R)—NH₂CH(CH₂CH₂CH₂CH₂CH₃)COOH. In some embodiments, a hydrophobic side chain is —CH₂R wherein R is optionally substituted phenyl. In some embodiments, R is phenyl. In some embodiments, R is phenyl substituted with one or more hydrocarbon group. In some embodiments, R is 4-phenylphenyl. In some embodiments, an amino acid with a hydrophobic side chain is NH₂CH(CH₂-4-phenylphenyl)COOH. In some embodiments, an amino acid with a hydrophobic side chain is (S)—NH₂CH(CH₂-4-phenylphenyl)COOH. In some embodiments, an amino acid with a hydrophobic side chain is (R)—NH₂CH(CH₂-4-phenylphenyl)COOH.

In some embodiments, an amino acid comprises a positively charged side chain (e.g., at physiological pH) as described herein. In some embodiments, such an amino acid comprises a basic nitrogen in its side chain. In some embodiments, such an amino acid is Arg, His or Lys. In some embodiments, such an amino acid is Arg. In some embodiments, such an amino acid is His. In some embodiments, such an amino acid is Lys.

In some embodiments, an amino acid comprises a negatively charged side chain (e.g., at physiological pH) as described herein. In some embodiments, such an amino acid comprises a —COOH in its side chain. In some embodiments, such an amino acid is Asp. In some embodiments, such an amino acid is Glu.

In some embodiments, an amino acid comprises a side chain comprising an aromatic group as described herein. In some embodiments, such an amino acid is Phe, Tyr, Trp, or His. In some embodiments, such an amino acid is Phe. In some embodiments, such an amino acid is Tyr. In some embodiments, such an amino acid is Trp. In some embodiments, such an amino acid is His. In some embodiments, such an amino acid is NH₂—CH(CH₂-4-phenylphenyl)-COOH. In some embodiments, such an amino acid is (S)—NH₂—CH(CH₂-4-phenylphenyl)-COOH. In some embodiments, such an amino acid is (R)—NH₂—CH(CH₂-4-phenylphenyl)-COOH.

In some embodiments, amino acids are known proteinogenic amino acids which are naturally encoded or found in the genetic code of any organism, or non-proteinogenic amino acids which are not naturally encoded or found in the genetic code of any organism. Examples of non-proteinogenic amino acids include α,α-disubstituted amino acids ((α-methylalanine etc.), N-alkyl-α-amino acids, and N-alkyl-α-D-amino acids, and those whose main chain structure may be different from the natural type. Examples of such amino acids include β-amino acids and amino acids having a side chain structure different from that of the natural type (such as norleucine, homohistidine, and hydroxyproline).

In some embodiments, an amino acid is selected from:

Bph β-(4-biphenylyl)-alanine

### MetO2 Methionine Sulfone

### Hse Homoserine

Har N6-carbamimidoyl-L-lysine

### da D-alanine

W6N (S)-2-amino-3-(1H-pyrrolo[2,3-c]pyridin-3-yl)propanoic acid
Ano (S)-2-aminononanoic acid
Ado (S)-2-aminoundecanoic acid
PhNle (S)-2-amino-6-phenylhexanoic acid
PhNva (S)-2-amino-5-phenylpentanoic acid

### Nal2 (2-Naphthyl)alanine

### Cit Citrulline

### F3G 3-Guanidinophenylalanine

RNMe N⁵-[imino(methylamino)methyl]-acetate-L-ornithine; CAS: 1135616-49-7

### RNNdMe N⁵-[(methylamino)(methylimino)methyl]-L-ornithine

### RNdMe N⁵-[(dimethylamino)iminomethyl]-L-ornithine; CAS: 1185841-84-2

hCit 2-Amino-5-(carbamoylamino) hexanoic acid; CAS: 201485-17-8
4Py2NH2 (S)-2-amino-3-(2-aminopyridin-4-yl) propanoic acid

### Target

In some embodiments, the present disclosure provides technologies for selectively directing agents comprising target binding moieties (e.g. ARM compounds), antibodies, and immune cells, e.g., NK cells, to desired target sites comprising one or more targets. As those skilled in the art will appreciate, provided technologies are useful for various types of targets, particularly those comprising CD38.

In some embodiments, targets are damaged or defective tissues. In some embodiments, a target is a damaged tissue. In some embodiments, a target is a defective tissue. In some embodiments, a target is associated with a disease, disorder or condition, e.g., cancer, wound, etc. In some embodiments, a target is a tumor. In some embodiments, targets are or comprise diseased cells. In some embodiments, targets are or comprise cancer cells. In some embodiments, a target is a foreign object. In some embodiments, a target is or comprises an infectious agent. In some embodiments, a target is a microbe. In some embodiments, a target is or comprises bacteria. In some embodiments, a target is or comprises viruses. In some embodiments, targets comprise or express CD38.

In many embodiments, targets are tissues and/or cells associated with diseases, disorders or conditions, particularly various types of cancers. In some embodiments, targets are or comprise cells associated with conditions, disorders or diseases. In some embodiments, targets are or comprise cells associated with cancer. In some embodiments, cells comprise or express CD38. Among other things, the present disclosure provides technologies that are particularly useful for selectively targeting cancer cells comprising or expressing CD38 by the immune system through, e.g., recruitment antibodies (e.g., endogenous antibodies) and immune cells by using ARMs.

Target sites typically comprise one or more physical, chemical and/or biological markers, e.g., CD38, that can be utilized e.g., by target binding moieties of provided compounds (e.g., ARMs), for selectively recruiting antibodies and/or fragments thereof, and/or immune cells to targets.

In some embodiments, cells of target sites comprise one or more characteristic agents that are useful for targeting, e.g., CD38. In some embodiments, such agents are proteins and/or fragments thereof. In some embodiments, such agents are antigens that are associated with diseases, disorders or conditions. In some embodiments, target sites and/or cells thereof comprise and/or express CD38, which target binding moieties of provided ARMs can bind to.

### Linker Moieties

In some embodiments, antibody binding moieties are optionally connected to target binding moieties through linker moieties. Linker moieties of various types and/or for various purposes, e.g., those utilized in antibody-drug conjugates, etc., may be utilized in accordance with the present disclosure.

Linker moieties can be either bivalent or polyvalent. In some embodiments, a linker moiety is bivalent. In some embodiments, a linker is polyvalent and connecting more than two moieties.

In some embodiments, a linker moiety is L. In some embodiments, L is a covalent bond, or a bivalent or polyvalent optionally substituted, linear or branched C₁₋₁₀₀ group comprising one or more aliphatic, aryl, heteroaliphatic having 1-20 heteroatoms, heteroaromatic having 1-20 heteroatoms, or any combinations thereof, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or —[(—O—C(R′)₂—C(R′)₂—)ₙ]—, wherein n is 1-20. In some embodiments, each amino acid residue is independently a residue of an amino acid having the structure of formula A-I or a salt thereof. In some embodiments, each amino acid residue independently has the structure of —N(R^{a1})-L^{a1}-C(R²)(R^{a3})-L²-CO— or a salt form thereof.

In some embodiments, L is bivalent. In some embodiments, L is a bivalent or optionally substituted, linear or branched group selected from C₁₋₁₀₀ aliphatic and C₁₋₁₀₀ heteroaliphatic having 1-50 heteroatoms, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid or -[(—O—C(R′)₂—C(R′)₂—)ₙ]—.

In some embodiments, L is a covalent bond. In some embodiments, L is a bivalent optionally substituted, linear or branched C₁₋₁₀₀ aliphatic group wherein one or more methylene units of the group are optionally and independently replaced. In some embodiments, L is a bivalent optionally substituted, linear or branched C₆₋₁₀₀ arylaliphatic group wherein one or more methylene units of the group are optionally and independently replaced. In some embodiments, L is a bivalent optionally substituted, linear or branched C₅ 100 heteroarylaliphatic group having 1-20 hetereoatoms wherein one or more methylene units of the group are optionally and independently replaced. In some embodiments, L is a bivalent optionally substituted, linear or branched C₁₋₁₀₀ heteroaliphatic group having 1-20 heteroatoms wherein one or more methylene units of the group are optionally and independently replaced.

In some embodiments, a linker moiety (e.g., L) is or comprises one or more (e.g., 1, 2, 3, 4, 5, 6, 7, 8, 9, 10, 11, 12, 13, 14, 15, 16, 17, 18, 19, 20 or more) polyethylene glycol units. In some embodiments, a linker moiety is or comprises —(CR₂CR₂O)ₙ—, wherein each of R and n is independently as described in the present disclosure. In some embodiments, a linker moiety is or comprises —(CH₂CH₂O)ₙ—, wherein n is as described in the present disclosure. In some embodiments, one or more methylene units of L are independently replaced with —(CH₂CH₂O)ₙ—. In some embodiments, two or more methyelen units of L are independently replaced with —(CR₂CR₂O)ₙ— or —(CH₂CH₂O)ₙ—. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, n is 11. In some embodiments, n is 12. In some embodiments, n is 13. In some embodiments, n is 14. In some embodiments, n is 15. In some embodiments, n is 16. In some embodiments, n is 17. In some embodiments, n is 18. In some embodiments, n is 19. In some embodiments, n is 20.

In some embodiments, the number of —(CR₂CR₂O)— unit, or the number of —(CH₂CH₂O)— unit, in a linker moiety such as L is at least about 1-20, 2-20, 3-30, 4-20, 5-20, 6-20, 7-20, 8-20, 9-20, 10-20, 11-20, 12-20, 13-20, 14-20, 15-20, or about or at least about 1, 2, 3, 4, 5, 6, 7, 8, 9, 10, 11, 12, 13, 14, 15, 16, 17, 18, 19, 20. In some embodiments, it is about or at least about 1. In some embodiments, it is about or at least about 2 In some embodiments, it is about or at least about 3 In some embodiments, it is about or at least about 4 In some embodiments, it is about or at least about 5 In some embodiments, it is about or at least about 6 In some embodiments, it is about or at least about 7 In some embodiments, it is about or at least about 8 In some embodiments, it is about or at least about 9 In some embodiments, it is about or at least about 10 In some embodiments, it is about or at least about 11 In some embodiments, it is about or at least about 12 In some embodiments, it is about or at least about 13 In some embodiments, it is about or at least about 14 In some embodiments, it is about or at least about 15 In some embodiments, it is about or at least about 16 In some embodiments, it is about or at least about 17 In some embodiments, it is about or at least about 18 In some embodiments, it is about or at least about 19 In some embodiments, it is about or at least about 20.

In some embodiments, a linker moiety, e.g., L, comprises one or more —(CR₂CR₂O)ₙ— and/or —(CH₂CH₂O)ₙ— as described herein, and one or more amino acid residues.

In some embodiments, a linker moiety, or L, is or comprises

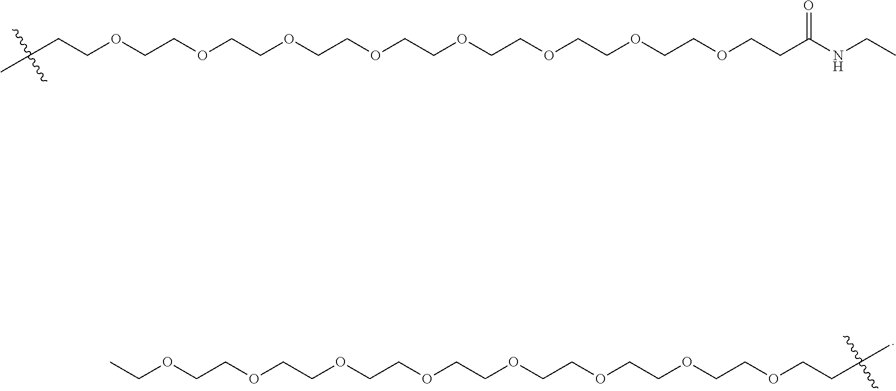

In some embodiments, a linker moiety, or L, is or comprises

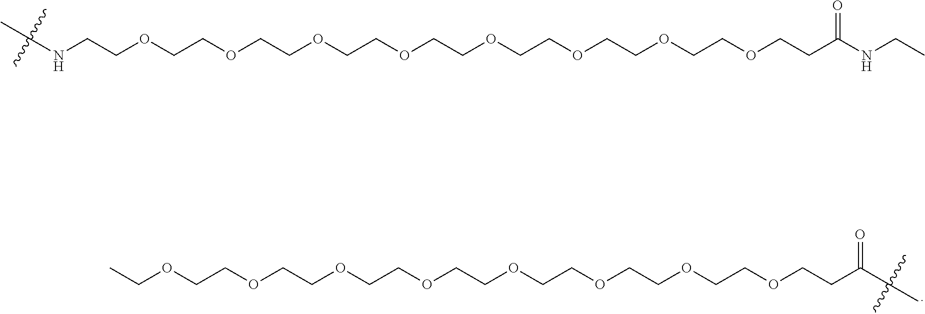

In some embodiments, a linker moiety, or L, is or comprises

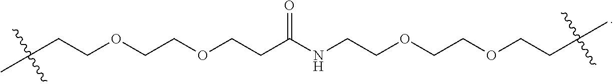 [SMILES: *CCOCCOCCNC(=O)CCOCCOCC*]

In some embodiments, a linker moiety, or L, is or comprises

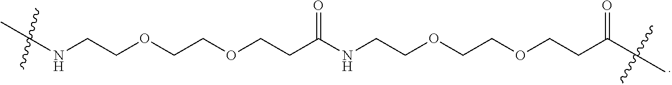 [SMILES: *NCCOCCOCCC(=O)NCCOCCOCCC(*)=O]

In some embodiments, a linker moiety, or L, is or comprises

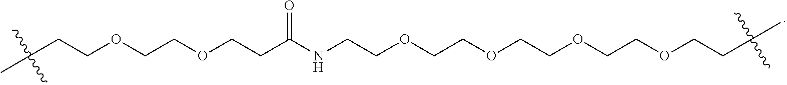 [SMILES: *CCOCCOCCOCCOCCNC(=O)CCOCCOCC*]

In some embodiments, a linker moiety, or L, is or comprises

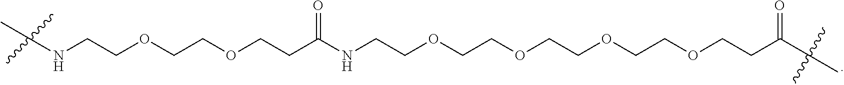 [SMILES: *NCCOCCOCCC(=O)NCCOCCOCCOCCOCCC(*)=O]

In some embodiments, a linker moiety, or L, is or comprises

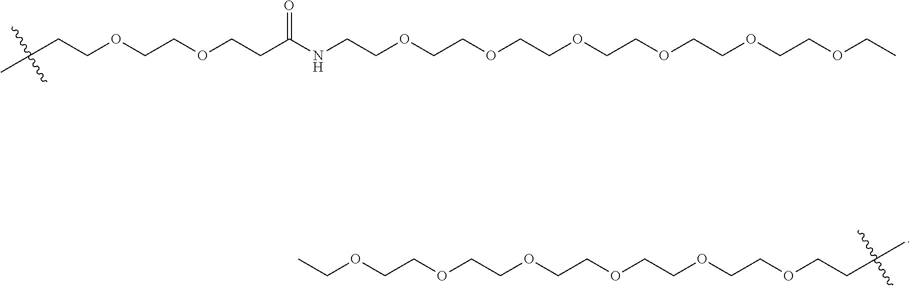

In some embodiments, a linker moiety, or L, is or comprises

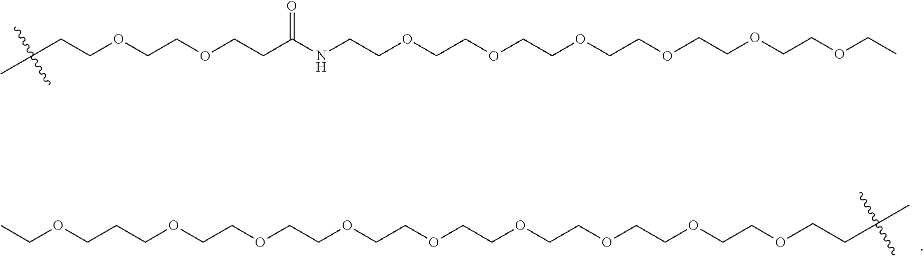

In some embodiments, a linker moiety, or L, is or comprises

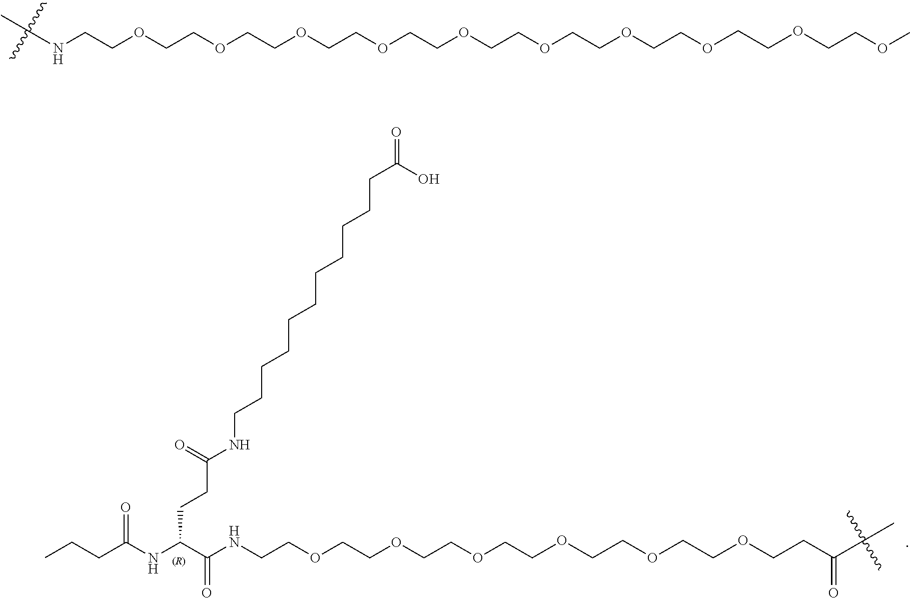

In some embodiments, a linker moiety, or L, is or comprises

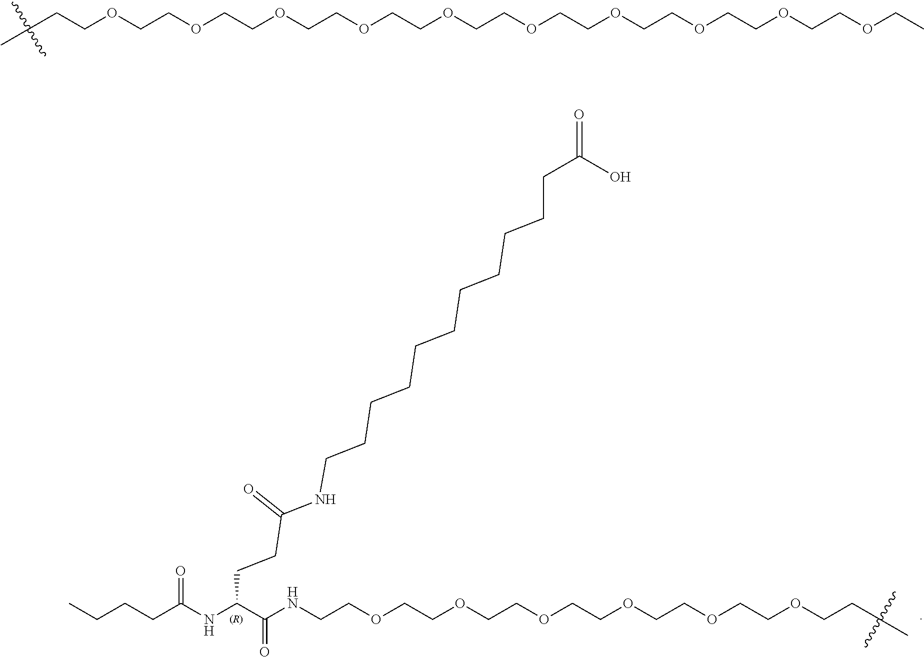

In some embodiments, a linker moiety (e.g., L) is or comprises one or more (e.g., 1, 2, 3, 4, 5, 6, 7, 8, 9, 10, 11, 12, 13, 14, 15, 16, 17, 18, 19, 20 or more) amino acid residues. As used in the present disclosure, “one or more” can be 1-100, 1-50, 1-40, 1-30, 1-20, 1-10, 1-5, 1, 2, 3, 4, 5, 6, 7, 8, 9, 10, 11, 12, 13, 14, 15, 16, 17, 18, 19, or 20 or more. In some embodiments, one or more methylene units of L are independently replaced with an amino acid residue. In some embodiments, one or more methylene units of L are independently replaced with an amino acid residue, wherein the amino acid residue is of an amino acid of formula A-I or a salt thereof. In some embodiments, one or more methylene units of L are independently replaced with an amino acid residue, wherein each amino acid residue independently has the structure of —N(R^{a1})-L^{a1}-C(R^{a2})(R^{a3})-L^{a2}-CO— or a salt form thereof. In some embodiments, an amino acid is a natural amino acid. In some embodiments, an amino acid is glycine. In some embodiments, an amino acid is an unnatural amino acid. In some embodiments, an amino acid is a D-amino acid. In some embodiments, an amino acid is beta-alanine. In some embodiments, an amino acid residue has the structure of —C(O)—(CH₂CH₂O)n-CH₂CH₂NR′— or a salt form thereof, wherein n is 0-20 (e.g., 0, 1, 2, 3, 4, 5, 6, 7, 8, 9, 10, 11, 12, 13, 14, 15, 16, 17, 18, 19 or 20), and R′ is as described herein. In some embodiments, n is 0. In some embodiments, n is 0-12. In some embodiments, n is 1-12. In some embodiments, R′ is —H.

In some embodiments, a linker moiety comprises one or more moieties, e.g., amino, carbonyl, etc., that can be utilized for connection with other moieties. In some embodiments, a linker moiety comprises one or more —NR′—, wherein R′ is as described in the present disclosure. In some embodiments, —NR′— improves solubility. In some embodiments, —NR′— serves as connection points to another moiety. In some embodiments, R′ is —H. In some embodiments, one or more methylene units of L are independently replaced with —NR′—, wherein R′ is as described in the present disclosure.

In some embodiments, a linker moiety, e.g., L, comprises a —C(O)— group, which can be utilized for connections with a moiety. In some embodiments, one or more methylene units of L are independently replaced with —C(O)—.

In some embodiments, a linker moiety, e.g., L, comprises a —NR′— group, which can be utilized for connections with a moiety. In some embodiments, one or more methylene units of L are independently replaced with —N(R′)—.

In some embodiments, a linker moiety, e.g., L, comprises a —C(O)NR′— group, which can be utilized for connections with a moiety. In some embodiments, one or more methylene units of L are independently replaced with —C(O)N(R′)—.

In some embodiments, a linker moiety, e.g., L, comprises a —C(R′)₂— group. In some embodiments, one or more methylene units of L are independently replaced with —C(R′)₂—. In some embodiments, —C(R′)₂— is —CHR′—. In some embodiments, R′ is —(CH₂)₂C(O)NH(CH₂)₁₁COOH. In some embodiments, R′ is —(CH₂)₂COOH. In some embodiments, R′ is —COOH.

In some embodiments, a linker moiety is or comprises one or more ring moieties, e.g., one or more methylene units of L are replaced with -Cy-. In some embodiments, a linker moiety, e.g., L, comprises an aryl ring. In some embodiments, a linker moiety, e.g., L, comprises an heteroaryl ring. In some embodiments, a linker moiety, e.g., L, comprises an aliphatic ring. In some embodiments, a linker moiety, e.g., L, comprises an heterocyclyl ring. In some embodiments, a linker moiety, e.g., L, comprises a polycyclic ring. In some embodiments, a ring in a linker moiety, e.g., L, is 3-20 membered. In some embodiments, a ring is 5-membered. In some embodiments, a ring is 6-membered. In some embodiments, a ring in a linker is product of a cycloaddition reaction (e.g., click chemistry, and variants thereof) utilized to link different moieties together.

In some embodiments, a linker moiety (e.g., L) is or comprises

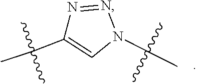 [SMILES: *c1cn(*)nn1]

In some embodiments, a methylene unit of L is replaced with

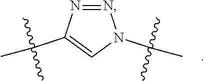 [SMILES: *c1cn(*)nn1]

In some embodiments, -Cy- is

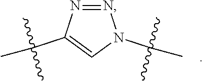 [SMILES: *c1cn(*)nn1]

In some embodiments, a linker moiety (e.g., L) is or comprises -Cy-. In some embodiments, a methylene unit of L is replaced with -Cy-. In some embodiments, -Cy- is

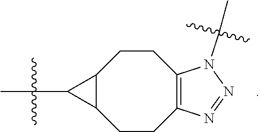 [SMILES: *C1C2CCc3nnn(*)c3CCC12]

In some embodiments, -Cy- is

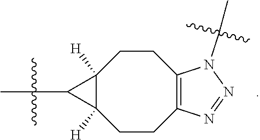 [SMILES: *C1[C@H]2CCc3c(nnn3*)CC[C@@H]12]

In some embodiments, -Cy- is

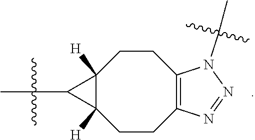 [SMILES: *C1[C@H]2CCc3nnn(*)c3CC[C@@H]12]

In some embodiments, a linker moiety, e.g., L, in a provided agent, e.g., a compound in Table 1, comprises

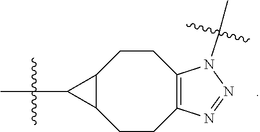 [SMILES: *C1C2CCc3nnn(*)c3CCC12]

In some embodiments,

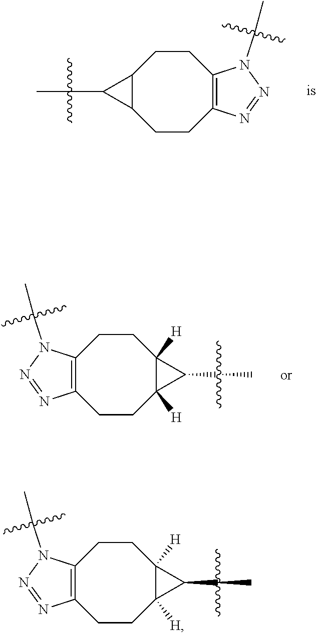

in the structure. In some embodiments,

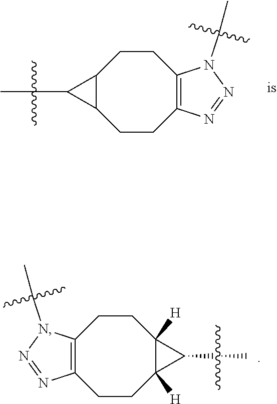

In some embodiments,

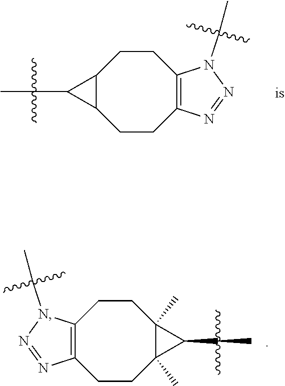 [SMILES: *C1C2CCc3nnn(*)c3CCC12 ; *[C@@H]1[C@]2(C)CCc3c(nnn3*)CC[C@]12C]

In some embodiments, a linker moiety is as described in Table 1. Additional linker moiety, for example, include those described for L². In some embodiments, L is L¹ ad present disclosure. In some embodiments, L is L² as described in the present disclosure. In some embodiments, L is L³ as described in the present disclosure. In some embodiments, L is L^{b} as described in the present disclosure.

In some embodiments, L is

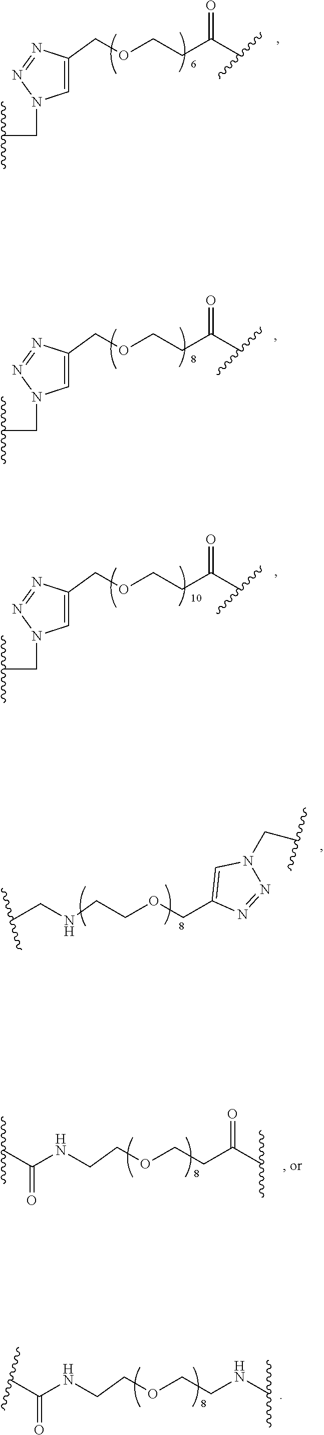

In some embodiments, a linker comprises an amino acid sequence comprising one or more amino acid residues. In some embodiments, a linker is or comprises

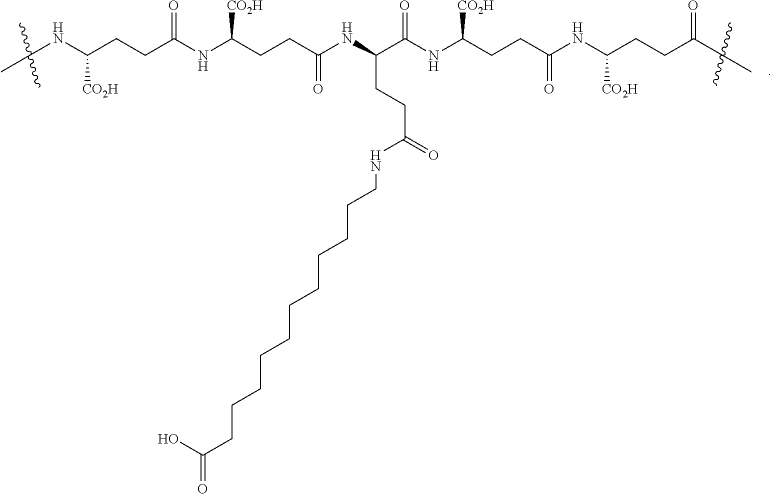 [SMILES: *N[C@H](CCC(=O)N[C@H](CCC(=O)N[C@H](CCC(=O)NCCCCCCCCCCCC(=O)O)C(=O)N[C@H](CCC(=O)N[C@H](CCC(*)=O)C(=O)O)C(=O)O)C(=O)O)C(=O)O]

In some embodiments, a linker is or comprises

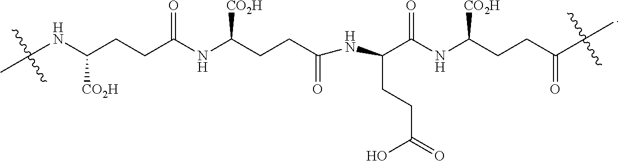 [SMILES: *N[C@H](CCC(=O)N[C@H](CCC(=O)N[C@H](CCC(=O)O)C(=O)N[C@H](CCC(*)=O)C(=O)O)C(=O)O)C(=O)O]

In some embodiments, a linker is or comprise a Gly residue. In some embodiments, a linker is or comprises -(Gly)n-, wherein n is as described herein. In some embodiments, n is 1-10, e.g., 1, 2, 3, 4, 5, 6, 7, 8, 9, or 10. In some embodiments, a linker is or comprises -Gly-Gly-. In some embodiments, a linker is or comprises -Gly-Gly-Gly-Gly-Gly-. Without the intention to be limited by theory, in some embodiments, linkers comprising amino acid residues may provide rigidity and/or orientation of various moieties that can encourage, promote and/or enhance one or more properties and/or activities.

In some embodiments, a linker connects to a moiety, e.g., a target binding moiety or a antibody binding moiety, through a N-terminal (e.g., through an amino group) or C-terminal amino acid residue (e.g., through a —COOH group). In some embodiments, a linker connects to a moiety through a side chain.

In some embodiments, a linker (e.g., L) is or comprises a bivalent optionally substituted C₁₋₂₀ aliphatic group. In some embodiments, a linker (e.g., L) is or comprises a bivalent optionally substituted C₁-20 alkylene group. In some embodiments, a bivalent group is linear. In some embodiments, a linker (e.g., L) is or comprises linear —(CH₂)ₙ—, wherein n is 1-20 (e.g., 1, 2, 3, 4, 5, 6, 7, 8, 9, 10, 11, 12, 13, 14, 15, 16, 17, 18, 19, or 20). In some embodiments, a linker (e.g., L) is or comprises a residue of an amino acid having the structure of NHR′—(CH₂)ₙ—COOH or a salt thereof. In some embodiments, a linker (e.g., L) is or comprises —NR′—(CH₂)ₙ—CO— or a salt form thereof. In some embodiments, R′ is —H. In some embodiments, as demonstrated herein, a linker comprises an albumin binding moiety.

In some embodiments, as used herein (e.g., in various moieties), n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, n is 11. In some embodiments, n is 12. In some embodiments, n is 13. In some embodiments, n is 14. In some embodiments, n is 15. In some embodiments, n is 16. In some embodiments, n is 17. In some embodiments, n is 18. In some embodiments, n is 19. In some embodiments, n is 20.

In some embodiments, a linker is or comprises a moiety, or a fragment thereof, that between two cyclic peptide moieties of a provided compound, e.g., in Table 1.

### Certain Embodiments of Variables

As examples, exemplary embodiments of variables are described throughout the present disclosure. As appreciated by those skilled in the art, embodiments for different variables may be optionally combined.

As defined above and described herein, ABT is an antibody binding moiety as described herein. In some embodiments, an ABT is an ABT of a compound selected from those depicted in Table 1, below. In some embodiments, an ABT is a moiety selected from Table A-1. In some embodiments, an ABT is a moiety described in Table 1.

In some embodiments, L is a bivalent or multivalent linker moiety linking one or more antibody binding moieties with one or more target binding moieties. In some embodiments, L is a bivalent linker moiety that connects ABT with TBT. In some embodiments, L is a multivalent linker moiety that connects ABT with TBT.

In some embodiments, L is a linker moiety of a compound selected from those depicted in Table 1, below.

As defined above and described herein, TBT is a target binding moiety as described herein.

In some embodiments, TBT is a target binding moiety of a compound selected from those depicted in Table 1, below. In some embodiments, a TBT is a moiety selected from Table T-1. In some embodiments, an TBT is a moiety described in Table 1.

As defined above and described herein, each of R¹, R³ and R⁵ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or: R¹ and R^{1′} are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen, oxygen, or sulfur; R³ and R^{3′} are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen, oxygen, or sulfur; an R⁵ group and the R^{5′} group attached to the same carbon atom are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen, oxygen, or sulfur; or two R⁵ groups are optionally taken together with their intervening atoms to form a C₁₋₁₀ bivalent straight or branched saturated or unsaturated hydrocarbon chain wherein 1-3 methylene units of the chain are independently and optionally replaced with —S—, —SS—, —N(R)—, —O—, —C(O)—, —OC(O)—, —C(O)O—, —C(O)N(R)—, —N(R)C(O)—, —S(O)—, —S(O)₂-, or -Cy¹-, wherein each -Cy¹- is independently a 5-6 membered heteroarylenyl with 1-4 heteroatoms independently selected from nitrogen, oxygen or sulfur.

In some embodiments, R¹ is hydrogen. In some embodiments, R¹ is optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur. In some embodiments, R¹ is an optionally substituted C₁₋₆ aliphatic group. In some embodiments, R¹ is an optionally substituted 3-8 membered saturated or partially unsaturated monocyclic carbocyclic ring. In some embodiments, R¹ is an optionally substituted phenyl. In some embodiments, R¹ is an optionally substituted 8-10 membered bicyclic aromatic carbocyclic ring. In some embodiments, R¹ is an optionally substituted 4-8 membered saturated or partially unsaturated monocyclic heterocyclic ring having 1-2 heteroatoms independently selected from nitrogen, oxygen, or sulfur. In some embodiments, R¹ is an optionally substituted 5-6 membered monocyclic heteroaromatic ring having 1-4 heteroatoms independently selected from nitrogen, oxygen, or sulfur. In some embodiments, R¹ is an optionally substituted 8-10 membered bicyclic heteroaromatic ring having 1-5 heteroatoms independently selected from nitrogen, oxygen, or sulfur.

In some embodiments, R¹ is

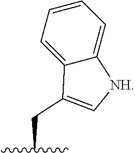

In some embodiments, R¹ is

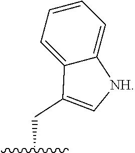

In some embodiments, R¹ is

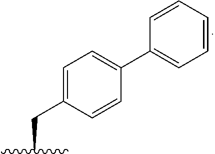

In some embodiments, R¹ is

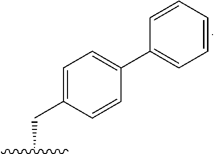

In some embodiments, R¹ is

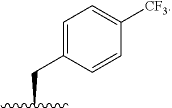

In some embodiments, R¹ is

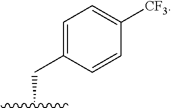

In some embodiments, R¹ is

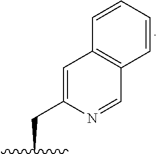

In some embodiments, R¹ is

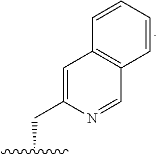

In some embodiments, R¹ is

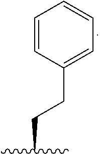

In some embodiments, R¹ is

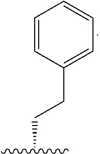

In some embodiments, R¹ is

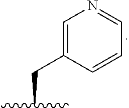

In some embodiments, R¹ is

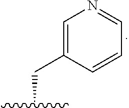

In some embodiments, R¹ is

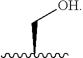

In some embodiments, R¹ is

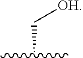

In some embodiments, R¹ is

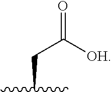

In some embodiments, R¹ is

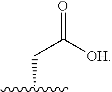

In some embodiments, R¹ is

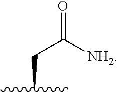

In some embodiments, R¹ is

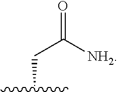

In some embodiments, R¹ is

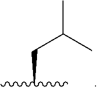

In some embodiments, R¹ is

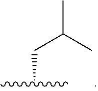

In some embodiments, R¹ and R^{1′} are optionally taken together with their intervening carbon atom to form a 3-8 membered saturated or partially unsaturated spirocyclic carbocyclic ring. In some embodiments, R¹ and R^{1′} are optionally taken together with their intervening carbon atom to form a 4-8 membered saturated or partially unsaturated spirocyclic heterocyclic ring having 1-2 heteroatoms independently selected from nitrogen, oxygen, or sulfur.

In some embodiments, R¹ is selected from those depicted in Table 1, below.

In some embodiments, R is R¹ as described in the present disclosure. In some embodiments, R^{a2} is R¹ as described in the present disclosure. In some embodiments, R^{a3} is R¹ as described in the present disclosure.

In some embodiments, R³ is hydrogen. In some embodiments, R³ is optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur. In some embodiments, R³ is an optionally substituted C₁₋₆ aliphatic group. In some embodiments, R³ is an optionally substituted 3-8 membered saturated or partially unsaturated monocyclic carbocyclic ring. In some embodiments, R³ is an optionally substituted phenyl. In some embodiments, R³ is an optionally substituted 8-10 membered bicyclic aromatic carbocyclic ring. In some embodiments, R³ is an optionally substituted 4-8 membered saturated or partially unsaturated monocyclic heterocyclic ring having 1-2 heteroatoms independently selected from nitrogen, oxygen, or sulfur. In some embodiments, R³ is an optionally substituted 5-6 membered monocyclic heteroaromatic ring having 1-4 heteroatoms independently selected from nitrogen, oxygen, or sulfur. In some embodiments, R³ is an optionally substituted 8-10 membered bicyclic heteroaromatic ring having 1-5 heteroatoms independently selected from nitrogen, oxygen, or sulfur.

In some embodiments, R³ is methyl. In some embodiments, R³ is

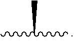

In some embodiments, R³ is

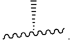

In some embodiments, R³ is

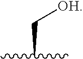

In some embodiments, R³ is

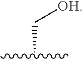

In some embodiments, R³ is

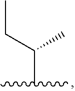 [SMILES: *[C@@H](C)CC]

wherein the site of attachment has (S) stereochemistry. In some embodiments, R³ is

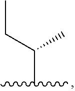 [SMILES: *[C@@H](C)CC]

wherein the site of attachment has (R) stereochemistry. In some embodiments, R³ is

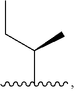 [SMILES: *[C@H](C)CC]

wherein the site of attachment has (S) stereochemistry. In some embodiments, R³ is

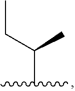 [SMILES: *[C@H](C)CC]

wherein the site of attachment has (R) stereochemistry.

In some embodiments, R³ is

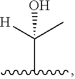 [SMILES: *[C@@H](C)O]

wherein the site of attachment has (S) stereochemistry. In some embodiments, R³ is

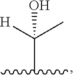 [SMILES: *[C@@H](C)O]

wherein the site of attachment has (R) stereochemistry.

In some embodiments, R³ and R^{3′} are optionally taken together with their intervening carbon atom to form a 3-8 membered saturated or partially unsaturated spirocyclic carbocyclic ring. In some embodiments, R³ and R^{3′} are optionally taken together with their intervening carbon atom to form a 4-8 membered saturated or partially unsaturated spirocyclic heterocyclic ring having 1-2 heteroatoms independently selected from nitrogen, oxygen, or sulfur.

In some embodiments, R³ is selected from those depicted in Table 1, below.

In some embodiments, R is R² as described in the present disclosure. In some embodiments, R^{a2} is R² as described in the present disclosure. In some embodiments, R^{a3} is R² as described in the present disclosure.

In some embodiments, R⁵ is hydrogen. In some embodiments, R⁵ is optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur. In some embodiments, R⁵ is an optionally substituted C₁₋₆ aliphatic group. In some embodiments, R⁵ is an optionally substituted 3-8 membered saturated or partially unsaturated monocyclic carbocyclic ring. In some embodiments, R⁵ is an optionally substituted phenyl. In some embodiments, R⁵ is an optionally substituted 8-10 membered bicyclic aromatic carbocyclic ring. In some embodiments, R⁵ is an optionally substituted 4-8 membered saturated or partially unsaturated monocyclic heterocyclic ring having 1-2 heteroatoms independently selected from nitrogen, oxygen, or sulfur. In some embodiments, R⁵ is an optionally substituted 5-6 membered monocyclic heteroaromatic ring having 1-4 heteroatoms independently selected from nitrogen, oxygen, or sulfur. In some embodiments, R⁵ is an optionally substituted 8-10 membered bicyclic heteroaromatic ring having 1-5 heteroatoms independently selected from nitrogen, oxygen, or sulfur.

In some embodiments, R⁵ is methyl. In some embodiments, R⁵ is

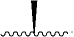

In some embodiments, R⁵ is

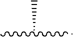

In some embodiments, R⁵ is

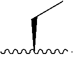

In some embodiments, R⁵ is

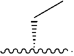

In some embodiments, R⁵ is

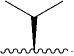

In some embodiments, R⁵ is

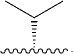

In some embodiments, R⁵ is

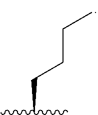

In some embodiments, R⁵ is

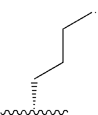

In some embodiments, R⁵ is

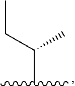 [SMILES: *[C@@H](C)CC]

wherein the site of attachment has (S) stereochemistry. In some embodiments, R⁵ is

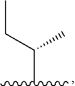 [SMILES: *[C@@H](C)CC]

wherein the site of attachment has (R) stereochemistry. In some embodiments, R⁵ is

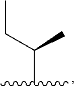 [SMILES: *[C@H](C)CC]

wherein the site of attachment has (S) stereochemistry. In some embodiments, R⁵ is

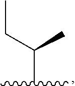 [SMILES: *[C@H](C)CC]

wherein the site of attachment has (R) stereochemistry. In some embodiments, R⁵ is

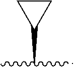

In some embodiments, R⁵ is

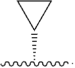

In some embodiments, R⁵ is

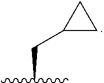

In some embodiments, R⁵ is

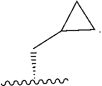

In some embodiments, R⁵ is

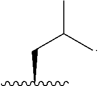

In some embodiments, R⁵ is

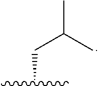

In some embodiments, R⁵ is

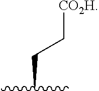

In some embodiments, R⁵ is

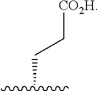

In some embodiments, R⁵ is

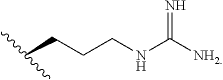

In some embodiments, R⁵ is

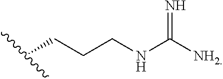

In some embodiments, R⁵ is

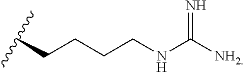

In some embodiments, R⁵ is

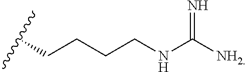

In some embodiments, R⁵ is

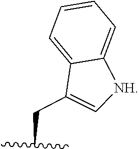

In some embodiments, R⁵ is

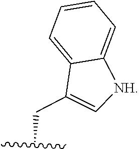

In some embodiments, R⁵ is

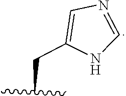

In some embodiments, R⁵ is

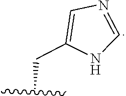

In some embodiments, R⁵ is

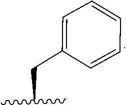

In some embodiments, R⁵ is

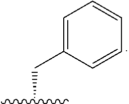

In some embodiments, R⁵ is

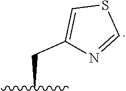

In some embodiments, R⁵ is

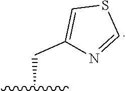

In some embodiments, R⁵ is

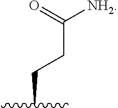

In some embodiments, R⁴ is 5

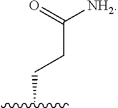

In some embodiments, R⁵ is

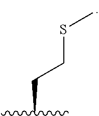

In some embodiments, R⁵ is

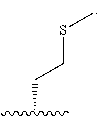

In some embodiments, R⁵ is

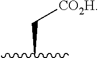

In some embodiments, R⁵ is

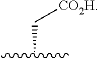

In some embodiments, R⁴ is

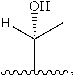 [SMILES: *[C@@H](C)O]

wherein the site of attachment has (S) stereochemistry. In some embodiments, R⁴ is

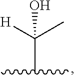 [SMILES: *[C@@H](C)O]

wherein the site of attachment has (R) stereochemistry.

In some embodiments, R⁵ and the R^{5′} group attached to the same carbon atom are optionally taken together with their intervening carbon atom to form a 3-8 membered saturated or partially unsaturated spirocyclic carbocyclic ring. In some embodiments, R⁵ and the R^{5′} group attached to the same carbon atom are optionally taken together with their intervening carbon atom to form a 4-8 membered saturated or partially unsaturated spirocyclic heterocyclic ring having 1-2 heteroatoms independently selected from nitrogen, oxygen, or sulfur.

In some embodiments, two R⁵ groups are taken together with their intervening atoms to form a C₁-10 bivalent straight or branched saturated or unsaturated hydrocarbon chain wherein 1-3 methylene units of the chain are independently and optionally replaced with —S—, —SS—, —N(R)—, —O—, —C(O)—, —OC(O)—, —C(O)O—, —C(O)N(R)—, —N(R)C(O)—, —S(O)—, —S(O)₂—, or -Cy¹-, wherein each -Cy¹- is independently a 5-6 membered heteroarylenyl with 1-4 heteroatoms independently selected from nitrogen, oxygen or sulfur.

In some embodiments, two R⁵ groups are taken together with their intervening atoms to form

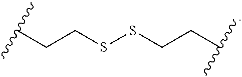 [SMILES: *CCSSCC*]

In some embodiments, two R⁵ groups are taken together with their intervening atoms to form

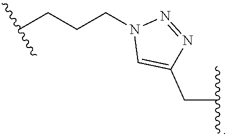 [SMILES: *CCCn1cc(C*)nn1]

In some embodiments, two R⁵ groups are taken together with their intervening atoms to form

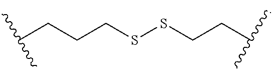 [SMILES: *CCCSSCC*]

In some embodiments, two R⁵ groups are taken together with their intervening atoms to form

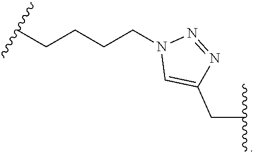 [SMILES: *CCCCn1cc(C*)nn1]

In some embodiments, R⁵ is selected from those depicted in Table 1, below.

In some embodiments, R is R⁵ as described in the present disclosure. In some embodiments, R^{a2} is R⁵ as described in the present disclosure. In some embodiments, R^{a3} is R⁵ as described in the present disclosure.

As defined above and described herein, each of R¹, R^{3′} and R^{5′} is independently hydrogen or C₁ 3 aliphatic.

In some embodiments, R^{1′} is hydrogen. In some embodiments, R^{1′} is C₁₋₃ aliphatic.

In some embodiments, R^{1′} is methyl. In some embodiments, R^{1′} is ethyl. In some embodiments, R^{1′} is n-propyl. In some embodiments, R^{1′} is isopropyl. In some embodiments, R^{1′} is cyclopropyl.

In some embodiments, R^{1′} is selected from those depicted in Table 1, below.

In some embodiments, R^{3′} is hydrogen. In some embodiments, R^{3′} is C₁₋₃ aliphatic.

In some embodiments, R^{3′} is methyl. In some embodiments, R^{3′} is ethyl. In some embodiments, R^{3′} is n-propyl. In some embodiments, R^{3′} is isopropyl. In some embodiments, R^{3′} is cyclopropyl.

In some embodiments, R^{3′} is selected from those depicted in Table 1, below.

In some embodiments, R^{5′} is hydrogen. In some embodiments, R^{5′} is C₁₋₃ aliphatic.

In some embodiments, R^{5′} is methyl. In some embodiments, R^{5′} is ethyl. In some embodiments, R^{5′} is n-propyl. In some embodiments, R^{5′} is isopropyl. In some embodiments, R^{5′} is cyclopropyl.

In some embodiments, R^{5′} is selected from those depicted in Table 1, below.

As defined above and described herein, each of R², R⁴ and R⁶ is independently hydrogen, or C₁₋₄ aliphatic, or: R² and R¹ are optionally taken together with their intervening atoms to form a 4-8 membered saturated or partially unsaturated monocyclic heterocyclic ring having 1-2 heteroatoms independently selected from nitrogen, oxygen, or sulfur; R⁴ and R³ are optionally taken together with their intervening atoms to form a 4-8 membered saturated or partially unsaturated monocyclic heterocyclic ring having 1-2 heteroatoms independently selected from nitrogen, oxygen, or sulfur; or an R⁶ group and its adjacent R⁵ group are optionally taken together with their intervening atoms to form a 4-8 membered saturated or partially unsaturated monocyclic heterocyclic ring having 1-2 heteroatoms independently selected from nitrogen, oxygen, or sulfur.

In some embodiments, R² is hydrogen. In some embodiments, R² is C₁₋₄ aliphatic. In some embodiments, R² is methyl. In some embodiments, R² is ethyl. In some embodiments, R² is n-propyl. In some embodiments, R² is isopropyl. In some embodiments, R² is n-butyl. In some embodiments, R² is isobutyl. In some embodiments, R² is tert-butyl.

In some embodiments, R² and R¹ are taken together with their intervening atoms to form a 4-8 membered saturated or partially unsaturated monocyclic heterocyclic ring having 1-2 heteroatoms independently selected from nitrogen, oxygen, or sulfur.

In some embodiments, R² and R¹ are taken together with their intervening atoms to form

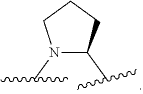 [SMILES: *[C@@H]1CCCN1*]

In some embodiments, R² and R¹ are taken together with their intervening atoms to form

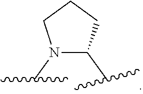 [SMILES: *[C@H]1CCCN1*]

In some embodiments, R² is selected from those depicted in Table 1, below.

In some embodiments, R⁴ is hydrogen. In some embodiments, R⁴ is C₁₋₄ aliphatic. In some embodiments, R⁴ is methyl. In some embodiments, R⁴ is ethyl. In some embodiments, R⁴ is n-propyl. In some embodiments, R⁴ is isopropyl. In some embodiments, R⁴ is n-butyl. In some embodiments, R⁴ is isobutyl. In some embodiments, R⁴ is tert-butyl.

In some embodiments, R⁴ and R³ are taken together with their intervening atoms to form a 4-8 membered saturated or partially unsaturated monocyclic heterocyclic ring having 1-2 heteroatoms independently selected from nitrogen, oxygen, or sulfur.

In some embodiments, R⁴ and R³ are taken together with their intervening atoms to form

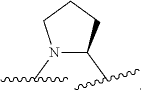 [SMILES: *[C@@H]1CCCN1*]

In some embodiments, R⁴ and R³ are taken together with their intervening atoms to form

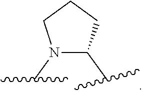 [SMILES: *[C@H]1CCCN1*]

In some embodiments, R⁴ is selected from those depicted in Table 1, below.

In some embodiments, R⁶ is hydrogen. In some embodiments, R⁶ is C₁₋₄ aliphatic. In some embodiments, R⁶ is methyl. In some embodiments, R⁶ is ethyl. In some embodiments, R⁶ is n-propyl. In some embodiments, R⁶ is isopropyl. In some embodiments, R⁶ is n-butyl. In some embodiments, R⁶ is isobutyl. In some embodiments, R⁶ is tert-butyl.

In some embodiments, an R⁶ group and its adjacent R⁵ group are taken together with their intervening atoms to form a 4-8 membered saturated or partially unsaturated monocyclic heterocyclic ring having 1-2 heteroatoms independently selected from nitrogen, oxygen, or sulfur.

In some embodiments, an R⁶ group and its adjacent R⁵ group are taken together with their intervening atoms to form

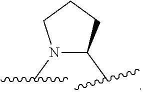 [SMILES: *[C@@H]1CCCN1*]

In some embodiments, an R⁶ group and its adjacent R⁵ group are taken together with their intervening atoms to form

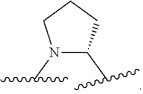 [SMILES: *[C@H]1CCCN1*]

In some embodiments, R⁶ is selected from those depicted in Table 1, below.

In some embodiments, R is R^{1′} as described in the present disclosure. In some embodiments, R² is R^{1′} as described in the present disclosure. In some embodiments, R^{a3} is R^{1′} as described in the present disclosure. In some embodiments, R is R^{3′} as described in the present disclosure. In some embodiments, R^{a2} is R^{3′} as described in the present disclosure. In some embodiments, R^{a3} is R^{3′} as described in the present disclosure. In some embodiments, R is R² as described in the present disclosure. In some embodiments, R^{a2} is R² as described in the present disclosure. In some embodiments, R^{a3} is R² as described in the present disclosure. In some embodiments, R is R⁴ as described in the present disclosure. In some embodiments, R^{a2} is R⁴ as described in the present disclosure. In some embodiments, R^{a3} is R⁴ as described in the present disclosure. In some embodiments, R is R⁶ as described in the present disclosure. In some embodiments, R^{a2} is R⁶ as described in the present disclosure. In some embodiments, R^{a3} is R⁶ as described in the present disclosure.

As defined above and described herein, L¹ is a trivalent linker moiety that connects

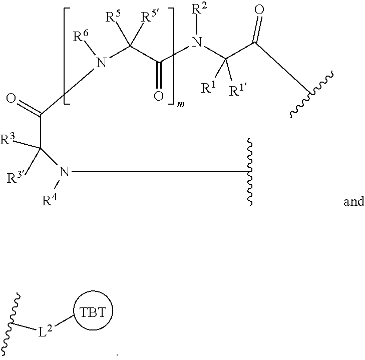

In some embodiments, L¹ is

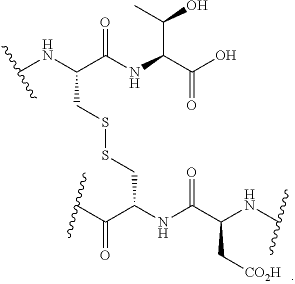 [SMILES: *N[C@@H](CC(=O)O)C(=O)N[C@@H](CSSC[C@H](N*)C(=O)N[C@H](C(=O)O)[C@@H](C)O)C(*)=O]

In some embodiments, L¹ is

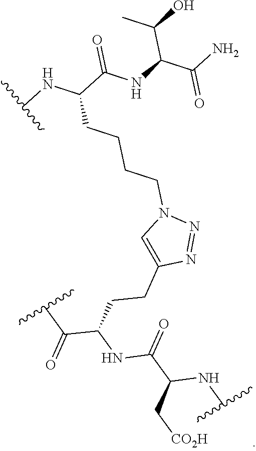 [SMILES: *N[C@@H](CC(=O)O)C(=O)N[C@@H](CCc1cn(CCCC[C@H](N*)C(=O)N[C@H](C(N)=O)[C@@H](C)O)nn1)C(*)=O]

In some embodiments, L¹ is

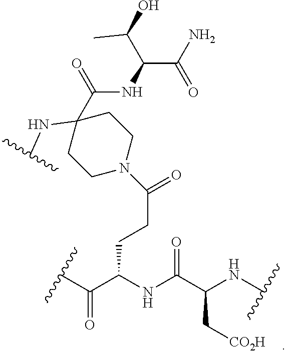 [SMILES: *N[C@@H](CC(=O)O)C(=O)N[C@@H](CCC(=O)N1CCC(N*)(C(=O)N[C@H](C(N)=O)[C@@H](C)O)CC1)C(*)=O]

In some embodiments, L¹ is

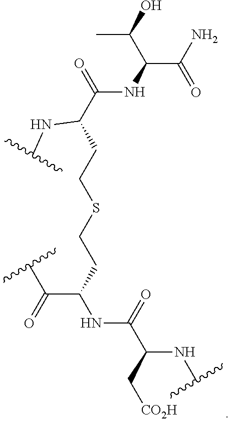 [SMILES: *N[C@@H](CC(=O)O)C(=O)N[C@@H](CCSCC[C@H](N*)C(=O)N[C@H](C(N)=O)[C@@H](C)O)C(*)=O]

In some embodiments, L¹ is

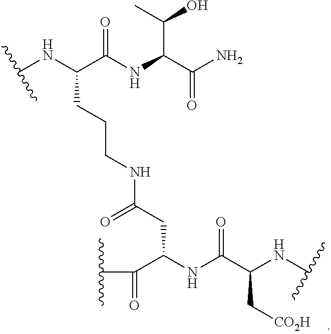 [SMILES: *N[C@@H](CC(=O)O)C(=O)N[C@@H](CC(=O)NCCC[C@H](N*)C(=O)N[C@H](C(N)=O)[C@@H](C)O)C(*)=O]

In some embodiments, L¹ is

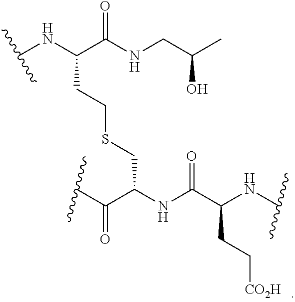 [SMILES: *N[C@@H](CCSC[C@H](NC(=O)[C@H](CCC(=O)O)N*)C(*)=O)C(=O)NC[C@@H](C)O]

In some embodiments, L¹ is

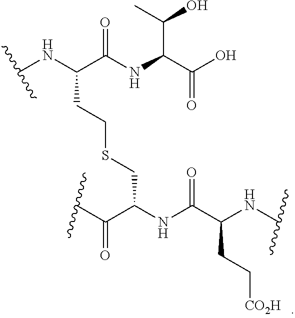 [SMILES: *N[C@@H](CCC(=O)O)C(=O)N[C@@H](CSCC[C@H](N*)C(=O)N[C@H](C(=O)O)[C@@H](C)O)C(*)=O]

In some embodiments, L¹ is

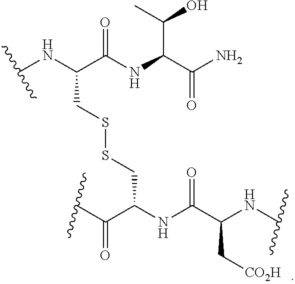 [SMILES: *N[C@@H](CC(=O)O)C(=O)N[C@@H](CSSC[C@H](N*)C(=O)N[C@H](C(N)=O)[C@@H](C)O)C(*)=O]

In some embodiments, L¹ is

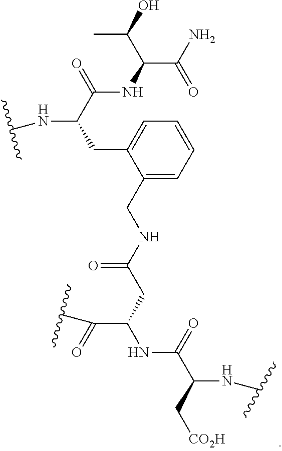 [SMILES: *N[C@@H](CC(=O)O)C(=O)N[C@@H](CC(=O)NCc1ccccc1C[C@H](N*)C(=O)N[C@H](C(N)=O)[C@@H](C)O)C(*)=O]

In some embodiments, L¹ is

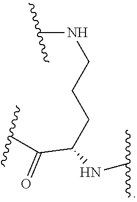 [SMILES: *NCCC[C@H](N*)C(*)=O]

In some embodiments, L¹ is

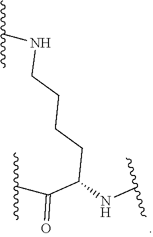 [SMILES: *NCCCC[C@H](N*)C(*)=O]

In some embodiments, L¹ is

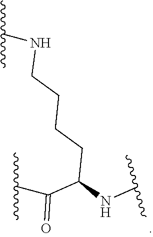 [SMILES: *NCCCC[C@@H](N*)C(*)=O]

In some embodiments, L¹ is

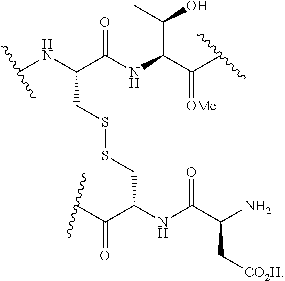

In some embodiments, L¹ is

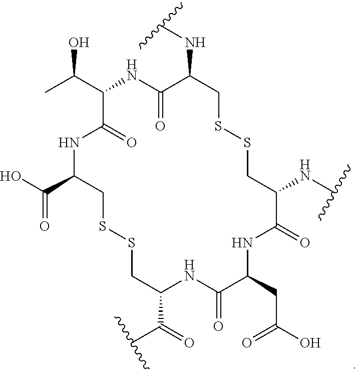 [SMILES: *N[C@H]1CSSC[C@H](N*)C(=O)N[C@@H]([C@@H](C)O)C(=O)N[C@H](C(=O)O)CSSC[C@@H](C(*)=O)NC(=O)[C@H](CC(=O)O)NC1=O]

In some embodiments, L¹ is

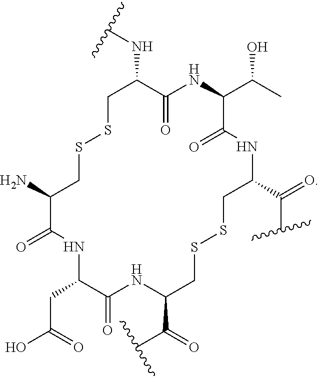 [SMILES: *N[C@H]1CSSC[C@H](N)C(=O)N[C@@H](CC(=O)O)C(=O)N[C@H](C(*)=O)CSSC[C@@H](C(*)=O)NC(=O)[C@H]([C@@H](C)O)NC1=O]

In some embodiments, L¹ is

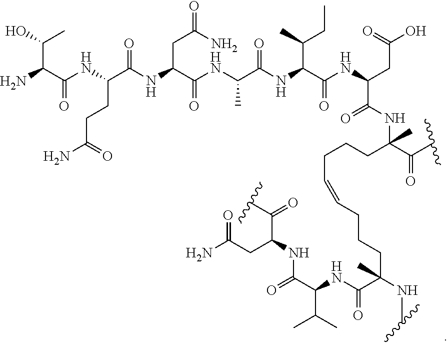 [SMILES: *N[C@@](C)(CCC/C=C\CCC[C@](C)(NC(=O)[C@H](CC(=O)O)NC(=O)[C@@H](NC(=O)[C@H](C)NC(=O)[C@H](CC(N)=O)NC(=O)[C@H](CCC(N)=O)NC(=O)[C@@H](N)[C@@H](C)O)[C@@H](C)CC)C(*)=O)C(=O)N[C@H](C(=O)N[C@@H](CC(N)=O)C(*)=O)C(C)C]

In some embodiments, L¹ is

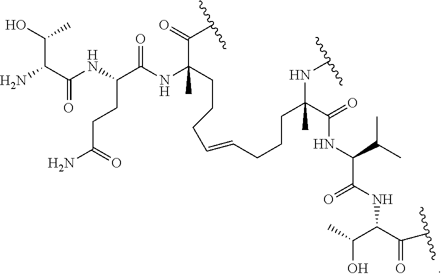 [SMILES: *N[C@@](C)(CCC/C=C/CCC[C@](C)(NC(=O)[C@H](CCC(N)=O)NC(=O)[C@H](N)[C@@H](C)O)C(*)=O)C(=O)N[C@H](C(=O)N[C@H](C(*)=O)[C@@H](C)O)C(C)C]

In some embodiments, L¹ is

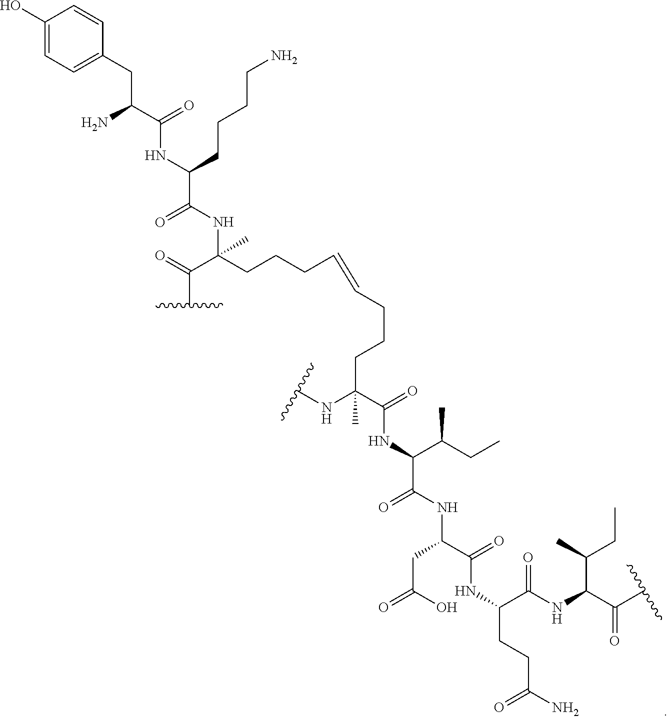 [SMILES: *N[C@@](C)(CCC/C=C/CCC[C@](C)(NC(=O)[C@H](CCCCN)NC(=O)[C@@H](N)Cc1ccc(O)cc1)C(*)=O)C(=O)N[C@H](C(=O)N[C@@H](CC(=O)O)C(=O)N[C@@H](CCC(N)=O)C(=O)N[C@H](C(*)=O)[C@@H](C)CC)[C@@H](C)CC]

In some embodiments, L¹ is

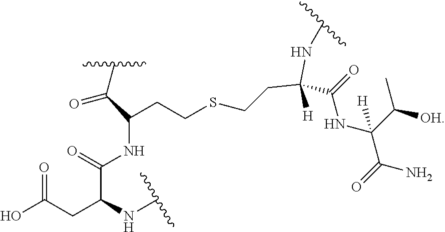

In some embodiments, L¹ is

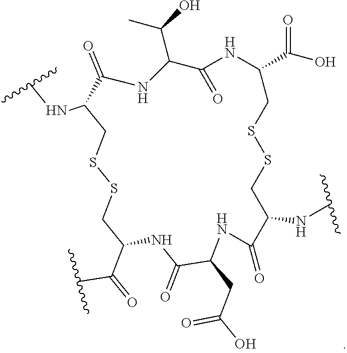 [SMILES: *N[C@H]1CSSC[C@@H](C(*)=O)NC(=O)[C@H](CC(=O)O)NC(=O)[C@@H](N*)CSSC[C@@H](C(=O)O)NC(=O)C([C@@H](C)O)NC1=O]

In some embodiments, L¹ is

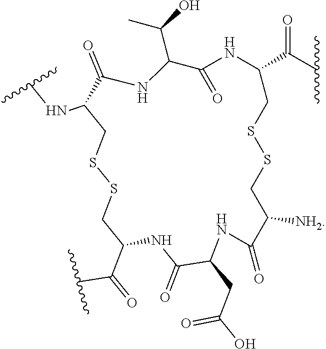 [SMILES: *N[C@H]1CSSC[C@@H](C(*)=O)NC(=O)[C@H](CC(=O)O)NC(=O)[C@@H](N)CSSC[C@@H](C(*)=O)NC(=O)C([C@@H](C)O)NC1=O]

In some embodiments, L¹ is

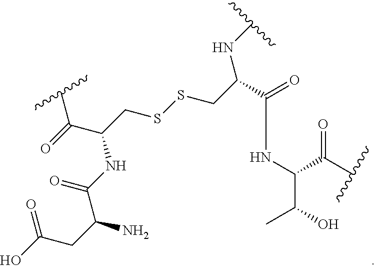 [SMILES: *N[C@@H](CSSC[C@H](NC(=O)[C@@H](N)CC(=O)O)C(*)=O)C(=O)N[C@H](C(*)=O)[C@@H](C)O]

In some embodiments, L¹ is selected from those depicted in Table 1, below.

As defined above and described herein, L² is a covalent bond or a C₁-10 bivalent straight or branched saturated or unsaturated hydrocarbon chain wherein 1-3 methylene units of the chain are independently and optionally replaced with —S—, —N(R)—, —O—, —C(O)—, —OC(O)—, —C(O)O—, —C(O)N(R)—, —N(R)C(O)—, —S(O)—, —S(O)₂—,

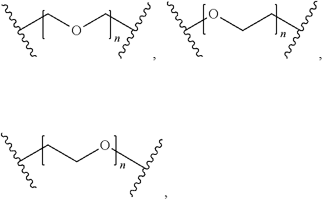

or -Cy¹-, wherein each -Cy¹- is independently a 5-6 membered heteroarylenyl with 1-4 heteroatoms independently selected from nitrogen, oxygen or sulfur.

In some embodiments, L² is a covalent bond. In some embodiments, L² is a C₁-10 bivalent straight or branched saturated or unsaturated hydrocarbon chain wherein 1-3 methylene units of the chain are independently and optionally replaced with —S—, —N(R)—, —O—, —C(O)—, —OC(O)—, —C(O)O—, —C(O)N(R)—, —N(R)C(O)—, —S(O)—, —S(O)₂—,

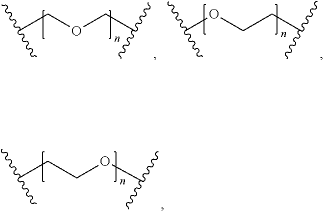

or -Cy¹-, wherein each -Cy¹- is independently a 5-6 membered heteroarylenyl with 1-4 heteroatoms independently selected from nitrogen, oxygen or sulfur.

In some embodiments, L² is

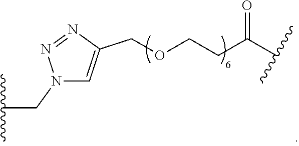

In some embodiments, L² is

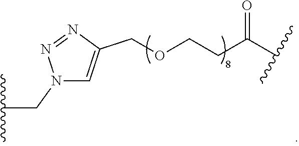

In some embodiments, L² is

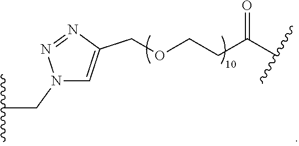

In some embodiments, L² is

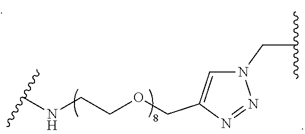

In some embodiments, L² is

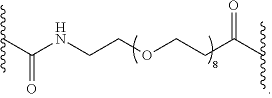

In some embodiments, L² is

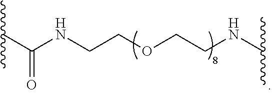

In some embodiments, L² is selected from those depicted in Table 1, below.

In some embodiments, L is L² as described in the present disclosure.

As defined above and described herein, TBT is a target binding moiety.

In some embodiments, TBT is a target binding moiety.

In some embodiments, TBT is

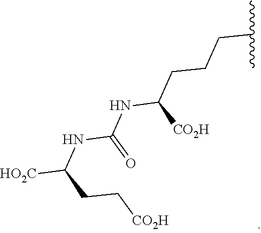 [SMILES: *CCC[C@H](NC(=O)N[C@@H](CCC(=O)O)C(=O)O)C(=O)O]

In some embodiments, TBT is

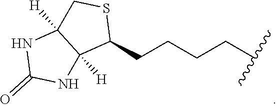 [SMILES: *CCCC[C@@H]1SC[C@@H]2NC(=O)N[C@@H]21]

In some embodiments, TBT is selected from those depicted in Table 1, below.

As defined above and described herein, each of m and n is independently 1, 2, 3, 4, 5, 6, 7, 8, 9, or 10.

In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10.

In some embodiments, m is selected from those depicted in Table 1, below.

In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In some embodiments, n is selected from those depicted in Table 1, below.

As defined above and described herein, each of R⁷ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or: an R⁷ group and the R^{7′} group attached to the same carbon atom are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen, oxygen, or sulfur.

In some embodiments, R⁷ is hydrogen. In some embodiments, R⁷ is optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur. In some embodiments, R⁷ is an optionally substituted C₁₋₆ aliphatic group. In some embodiments, R⁷ is an optionally substituted 3-8 membered saturated or partially unsaturated monocyclic carbocyclic ring. In some embodiments, R⁷ is an optionally substituted phenyl. In some embodiments, R⁷ is an optionally substituted 8-10 membered bicyclic aromatic carbocyclic ring. In some embodiments, R⁷ is an optionally substituted 4-8 membered saturated or partially unsaturated monocyclic heterocyclic ring having 1-2 heteroatoms independently selected from nitrogen, oxygen, or sulfur. In some embodiments, R⁷ is an optionally substituted 5-6 membered monocyclic heteroaromatic ring having 1-4 heteroatoms independently selected from nitrogen, oxygen, or sulfur. In some embodiments, R⁷ is an optionally substituted 8-10 membered bicyclic heteroaromatic ring having 1-5 heteroatoms independently selected from nitrogen, oxygen, or sulfur.

In some embodiments, R⁷ is methyl. In some embodiments, R⁷ is

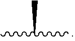

In some embodiments, R⁷ is

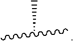

In some embodiments, R⁷ is

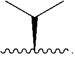

In some embodiments, R⁷ is

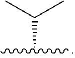

In some embodiments, R⁷ is

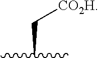

In some embodiments, R⁷ is

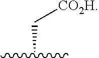

In some embodiments, R⁷ is

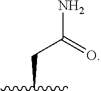

In some embodiments, R⁷ is

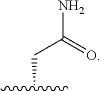

In some embodiments, R⁷ is

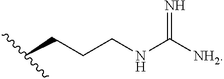

In some embodiments, R⁷ is

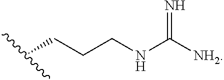

In some embodiments, R⁷ is

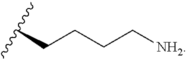

In some embodiments, R⁷ is

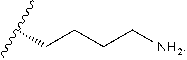

In some embodiments, R⁷ is

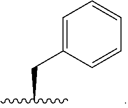

In some embodiments, R⁷ is

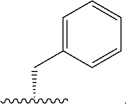

In some embodiments, R⁷ is

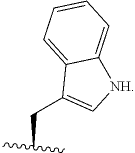

In some embodiments, R⁷ is

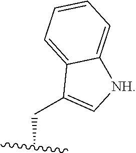

In some embodiments, R⁷ is

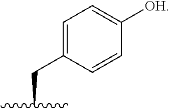

In some embodiments, R⁷ is

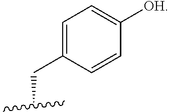

In some embodiments, R⁷ is

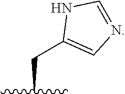

In some embodiments, R⁷ is

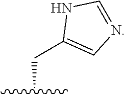

In some embodiments, an R⁷ group and the R^{7′} group attached to the same carbon atom are taken together with their intervening carbon atom to form a 3-8 membered saturated or partially unsaturated spirocyclic carbocyclic ring. In some embodiments, an R⁷ group and the R^{7′} group attached to the same carbon atom are taken together with their intervening carbon atom to form a 4-8 membered saturated or partially unsaturated spirocyclic heterocyclic ring having 1-2 heteroatoms independently selected from nitrogen, oxygen, or sulfur.

In some embodiments, R⁷ is selected from those depicted in Table 1, below.

As defined above and described herein, each of R^{7′} is independently hydrogen or C₁₋₃ aliphatic.

In some embodiments, R^{7′} is hydrogen. In some embodiments, R^{7′} is methyl. In some embodiments, R^{7′} is ethyl. In some embodiments, R^{7′} is n-propyl. In some embodiments, R^{7′} is isopropyl.

In some embodiments, R^{7′} is selected from those depicted in Table 1, below.

As defined above and described herein, each of R⁸ is independently hydrogen, or C₁₋₄ aliphatic, or: an R⁸ group and its adjacent R⁷ group are optionally taken together with their intervening atoms to form a 4-8 membered saturated or partially unsaturated monocyclic heterocyclic ring having 1-2 heteroatoms independently selected from nitrogen, oxygen, or sulfur.

In some embodiments, R⁸ is hydrogen. In some embodiments, R⁸ is C₁₋₄ aliphatic. In some embodiments, R⁸ is methyl. In some embodiments, R⁸ is ethyl. In some embodiments, R⁸ is n-propyl. In some embodiments, R⁸ is isopropyl. In some embodiments, R⁸ is n-butyl. In some embodiments, R⁸ is isobutyl. In some embodiments, R⁸ is tert-butyl.

In some embodiments, an R⁸ group and its adjacent R⁷ group are taken together with their intervening atoms to form a 4-8 membered saturated or partially unsaturated monocyclic heterocyclic ring having 1-2 heteroatoms independently selected from nitrogen, oxygen, or sulfur.

In some embodiments, an R⁸ group and its adjacent R⁷ group are taken together with their intervening atoms to form

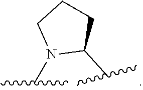 [SMILES: *[C@@H]1CCCN1*]

In some embodiments, an R⁸ group and its adjacent R⁷ group are taken together with their intervening atoms to form

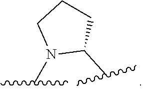 [SMILES: *[C@H]1CCCN1*]

In some embodiments, R⁸ is selected from those depicted in Table 1, below.

As defined above and described herein, R⁹ is hydrogen, C₁₋₃ aliphatic, or —C(O)C₁₋₃ aliphatic.

In some embodiments, R⁹ is hydrogen. In some embodiments, R⁹ is C₁₋₃ aliphatic. In some embodiments, R⁹ is —C(O)C₁ 3 aliphatic.

In some embodiments, R⁹ is methyl. In some embodiments, R⁹ is ethyl. In some embodiments, R⁹ is n-propyl. In some embodiments, R⁹ is isopropyl. In some embodiments, R⁹ is cyclopropyl.

In some embodiments, R⁹ is —C(O)Me. In some embodiments, R⁹ is —C(O)Et. In some embodiments, R⁹ is —C(O)CH₂CH₂CH₃. In some embodiments, R⁹ is —C(O)CH(CH₃)₂. In some embodiments, R⁹ is —C(O)cyclopropyl.

In some embodiments, R⁹ is selected from those depicted in Table 1, below.

In some embodiments, R is R⁷ as described in the present disclosure. In some embodiments, R^{a2} is R⁷ as described in the present disclosure. In some embodiments, R^{a3} is R⁷ as described in the present disclosure. In some embodiments, R is R^{7′} as described in the present disclosure. In some embodiments, R^{a2} is R^{7′} as described in the present disclosure. In some embodiments, R^{a3} is R^{7′} as described in the present disclosure. In some embodiments, R is R⁸ as described in the present disclosure. In some embodiments, R^{a2} is R⁸ as described in the present disclosure. In some embodiments, R^{a3} is R⁸ as described in the present disclosure. In some embodiments, R is R^{1′} as described in the present disclosure. In some embodiments, R^{a2} is R^{1′} as described in the present disclosure. In some embodiments, R^{a3} is R^{1′} as described in the present disclosure. In some embodiments, R is R⁹ as described in the present disclosure. In some embodiments, R^{a2} is R⁹ as described in the present disclosure. In some embodiments, R^{a3} is R⁹ as described in the present disclosure.

As defined above and described herein, L³ is a bivalent linker moiety that connects

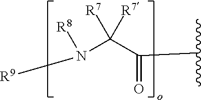

with TBT.

In some embodiments, L³ is a bivalent linker moiety that connects

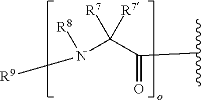

with TBT.

In some embodiments, L³ is

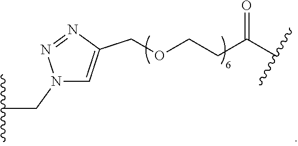

In some embodiments, L³ is

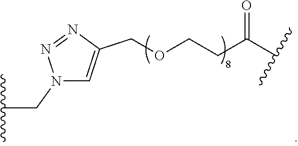

In some embodiments, L³ is

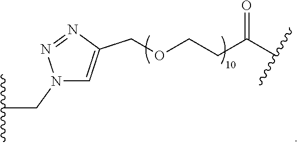

In some embodiments, L³ is

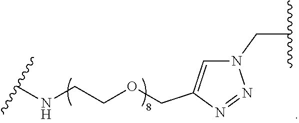

In some embodiments, L³ is

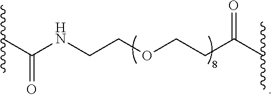

In some embodiments, L³ is

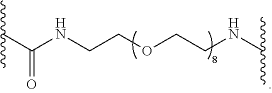

In some embodiments, L³ is selected from those depicted in Table 1, below.

In some embodiments, L is L³ as described in the present disclosure.

As defined above and described herein, o is 1, 2, 3, 4, 5, 6, 7, 8, 9, or 10.

In some embodiments, o is 1. In some embodiments, o is 2. In some embodiments, o is 3. In some embodiments, o is 4. In some embodiments, o is 5. In some embodiments, o is 6. In some embodiments, o is 7. In some embodiments, o is 8. In some embodiments, o is 9. In some embodiments, o is 10.

In some embodiments, o is selected from those depicted in Table 1, below.

In certain embodiments, the present disclosure provides a compound of formula II, wherein L² is

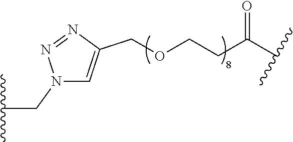

### and TBT is

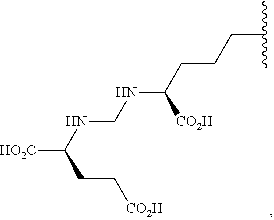 [SMILES: *CCC[C@H](NCN[C@@H](CCC(=O)O)C(=O)O)C(=O)O]

thereby forming a compound of formula II-a:

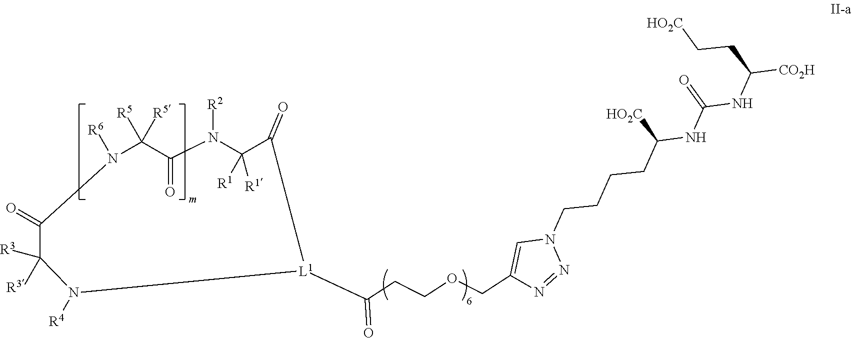

or a pharmaceutically acceptable salt thereof, wherein each of L¹, R¹, R^{1′}, R², R³, R^{3′}, R⁴, R⁵, R^{5′}, R⁶, and m is as defined above and described in embodiments herein, both singly and in combination.

In certain embodiments, the present disclosure provides a compound of formula II, wherein L² is

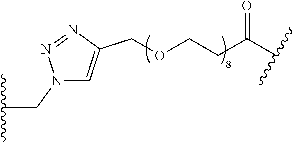

### and TBT is

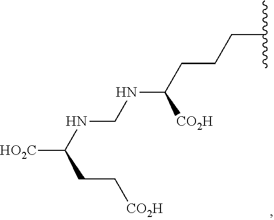 [SMILES: *CCC[C@H](NCN[C@@H](CCC(=O)O)C(=O)O)C(=O)O]

thereby forming a compound of formula II-b:

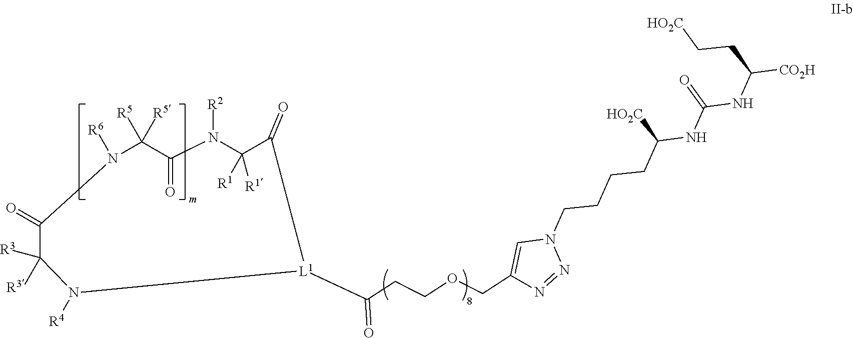

or a pharmaceutically acceptable salt thereof, wherein each of L¹, R¹, R^{1′}, R², R³, R^{3′}, R⁴, R⁵, R^{5′}, R⁶, and m is as defined above and described in embodiments herein, both singly and in combination.

In certain embodiments, the present disclosure provides a compound of formula II, wherein L² is

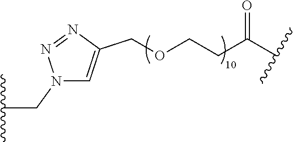

### and TBT is

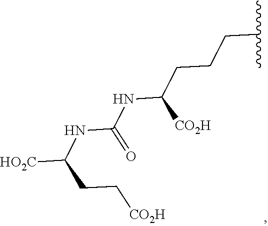 [SMILES: *CCC[C@H](NC(=O)N[C@@H](CCC(=O)O)C(=O)O)C(=O)O]

thereby forming a compound of formula II-c:

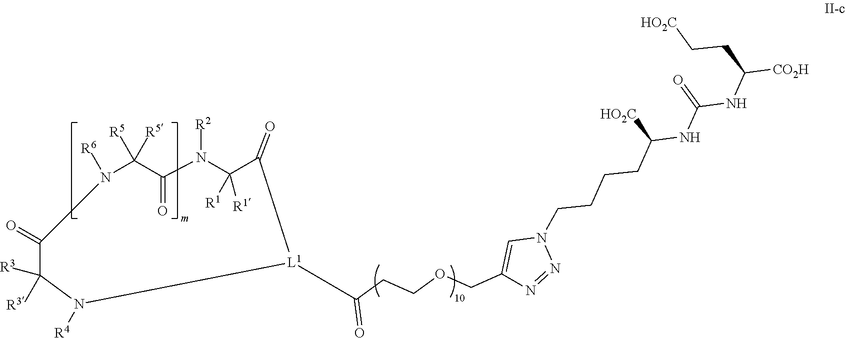

or a pharmaceutically acceptable salt thereof, wherein each of L¹, R¹, R^{1′}, R², R³, R^{3′}, R⁴, R⁵, R^{5′}, R⁶, and m is as defined above and described in embodiments herein, both singly and in combination.

In certain embodiments, the present disclosure provides a compound of formula II, wherein L² is

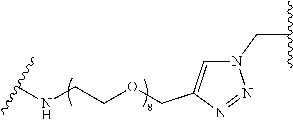

### and TBT is

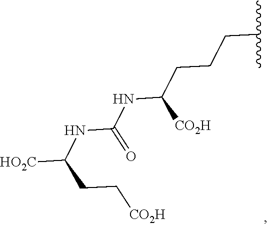 [SMILES: *CCC[C@H](NC(=O)N[C@@H](CCC(=O)O)C(=O)O)C(=O)O]

thereby forming a compound of formula II-d:

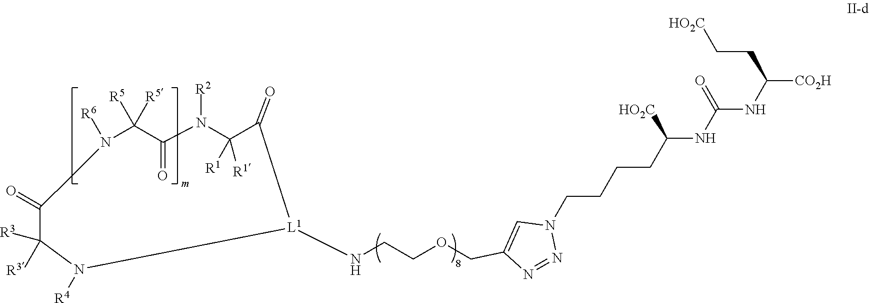

or a pharmaceutically acceptable salt thereof, wherein each of L¹, R¹, R^{1′}, R², R³, R^{3′}, R⁴, R⁵, R^{5′}, R⁶, and m is as defined above and described in embodiments herein, both singly and in combination.

In certain embodiments, the present disclosure provides a compound of formula II, wherein L² is

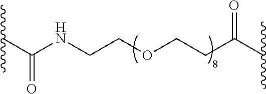

### and TBT is

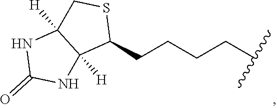 [SMILES: *CCCC[C@@H]1SC[C@@H]2NC(=O)N[C@@H]21]

thereby forming a compound of formula II-e:

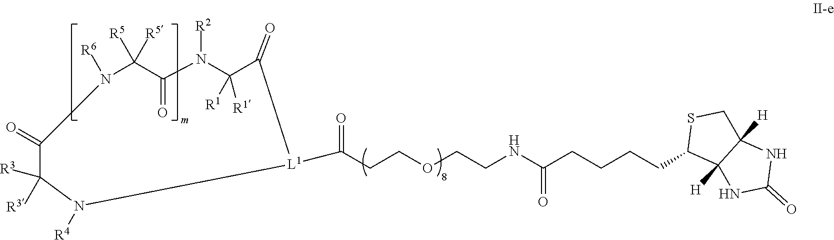

or a pharmaceutically acceptable salt thereof, wherein each of L¹, R¹, R^{1′}, R², R³, R^{3′}, R⁴, R⁵, R^{5′}, R⁶, and m is as defined above and described in embodiments herein, both singly and in combination.

In certain embodiments, the present disclosure provides a compound of formula II, wherein L² is

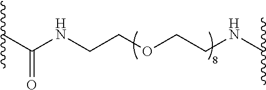

### and TBT is

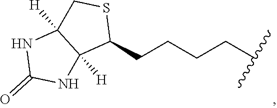 [SMILES: *CCCC[C@@H]1SC[C@@H]2NC(=O)N[C@@H]21]

thereby forming a compound of formula II-f:

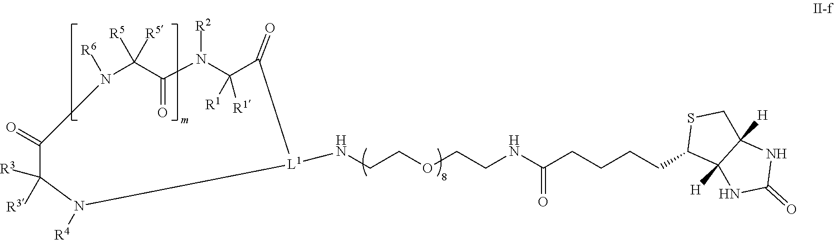

or a pharmaceutically acceptable salt thereof, wherein each of L¹, R¹, R^{1′}, R², R³, R^{3′}, R⁴, R⁵, R^{5′}, R⁶, and m is as defined above and described in embodiments herein, both singly and in combination.

In some embodiments, R^{a1} is R as described in the present disclosure. In some embodiments, R^{a1} is optionally substituted C₁₋₄ aliphatic. In some embodiments, R^{a1} is optionally substituted C₁₋₄ alkyl. In some embodiments, R^{a1} is methyl.

In some embodiments, L^{a1} is L^{a} as described in the present disclosure. In some embodiments, L^{a1} is a covalent bond.

In some embodiments, L^{a2} is L^{a} as described in the present disclosure. In some embodiments, L² is a covalent bond.

In some embodiments, L^{T} is L^{a} as described herein. In some embodiments, L^{T} is L as described herein. In some embodiments, L^{T} is a covalent bond. In some embodiments, L^{T} is —CH₂—C(O)—. In some embodiments, L^{T} links a —S— of a side chain (e.g., through —CH₂) with the amino group of an amino acid residue (e.g., through —C(O)—).

In some embodiments, L^{a} is a covalent bond. In some embodiments, L^{a} is an optionally substituted bivalent group selected from C₁-C₁₀ aliphatic or C₁-C₁₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{a} is an optionally substituted bivalent group selected from C₁-C₅ aliphatic or C₁-C₅ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —, -Cy-, —O—, —S—, —S—S—, —N(R′)—, —C(O)—, —C₂—, —S(O)₂N(R′)—, —C(O)S—, or —C(O)O—. In some embodiments, L^{a} is an optionally substituted bivalent C₁-C₅ aliphatic, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{a} is an optionally substituted bivalent C₁-C₅ aliphatic. In some embodiments, L^{a} is an optionally substituted bivalent C₁-C₅ heteroaliphatic having 1-3 heteroatoms independently selected from nitrogen, oxygen and sulfur.

In some embodiments, R^{a2} is R as described in the present disclosure. In some embodiments, R^{a2} is a side chain of a natural amino acid. In some embodiments, R^{a3} is R as described in the present disclosure. In some embodiments, R^{a3} is a side chain of a natural amino acid. In some embodiments, one of R^{2a} and R^{3a} is hydrogen. In some embodiments, R^{a2} and/or R^{a3} are R, wherein R is optionally substituted C₁₋₈ alphatic or aryl. In some embodiments, R is optionally substituted linear C₂₋₈ alkyl. In some embodiments, R is linear C₂₋₈ alkyl. In some embodiments, R is optionally substituted branched C₂₋₈ alkyl. In some embodiments, R is branched C₂₋₈ alkyl. In some embodiments, R is n-pentyl. In some embodiments, R is optionally substituted phenyl. In some embodiments, R is optionally substituted —CH₂-phenyl. In some embodiments, R is 4-phenylphenyl-CH₂—.

In some embodiments, each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon. In some embodiments, each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon. In some embodiments, -Cy- is an optionally substituted ring as described in the present disclosure, for example, for R and Cy^{L}, but is bivalent.

In some embodiments, -Cy- is monocyclic. In some embodiments, -Cy- is bicyclic. In some embodiments, -Cy- is polycyclic. In some embodiments, -Cy- is saturated. In some embodiments, -Cy- is partially unsaturated. In some embodiments, -Cy- is aromatic. In some embodiments, -Cy- comprises a saturated monocyclic moiety. In some embodiments, -Cy- comprises a partially unsaturated monocyclic moiety. In some embodiments, -Cy- comprises an aromatic monocyclic moiety. In some embodiments, -Cy- comprises a combination of a saturated, a partially unsaturated, and/or an aromatic cyclic moiety. In some embodiments, -Cy- is or comprises 3-membered ring. In some embodiments, -Cy- is or comprises 4-membered ring. In some embodiments, -Cy- is or comprises 5-membered ring. In some embodiments, -Cy- is or comprises 6-membered ring. In some embodiments, -Cy- is or comprises 7-membered ring. In some embodiments, -Cy- is or comprises 8-membered ring. In some embodiments, -Cy- is or comprises 9-membered ring. In some embodiments, -Cy- is or comprises 10-membered ring. In some embodiments, -Cy- is or comprises 11-membered ring. In some embodiments, -Cy- is or comprises 12-membered ring. In some embodiments, -Cy- is or comprises 13-membered ring. In some embodiments, -Cy- is or comprises 14-membered ring. In some embodiments, -Cy- is or comprises 15-membered ring. In some embodiments, -Cy- is or comprises 16-membered ring. In some embodiments, -Cy- is or comprises 17-membered ring. In some embodiments, -Cy- is or comprises 18-membered ring. In some embodiments, -Cy- is or comprises 19-membered ring. In some embodiments, -Cy- is or comprises 20-membered ring.

In some embodiments, -Cy- is or comprises an optionally substituted bivalent C₃₋₂₀ cycloaliphatic ring. In some embodiments, -Cy- is or comprises an optionally substituted bivalent, saturated C₃₋₂₀ cycloaliphatic ring. In some embodiments, -Cy- is or comprises an optionally substituted bivalent, partially unsaturated C₃₋₂₀ cycloaliphatic ring. In some embodiments, -Cy-H is optionally substituted cycloaliphatic as described in the present disclosure, for example, cycloaliphatic embodiments for R.

In some embodiments, -Cy- is or comprises an optionally substituted C₆₋₂₀ aryl ring. In some embodiments, -Cy- is or comprises optionally substituted phenylene. In some embodiments, -Cy- is or comprises optionally substituted 1,2-phenylene. In some embodiments, -Cy- is or comprises optionally substituted 1,3-phenylene. In some embodiments, -Cy- is or comprises optionally substituted 1,4-phenylene. In some embodiments, -Cy- is or comprises an optionally substituted bivalent naphthalene ring. In some embodiments, -Cy-H is optionally substituted aryl as described in the present disclosure, for example, aryl embodiments for R.

In some embodiments, -Cy- is or comprises an optionally substituted bivalent 5-20 membered heteroaryl ring having 1-10 heteroatoms independently selected from oxygen, nitrogen, sulfur, phosphorus and silicon. In some embodiments, -Cy- is or comprises an optionally substituted bivalent 5-20 membered heteroaryl ring having 1-10 heteroatoms independently selected from oxygen, nitrogen, and sulfur. In some embodiments, -Cy- is or comprises an optionally substituted bivalent 5-6 membered heteroaryl ring having 1-4 heteroatoms independently selected from oxygen, nitrogen, sulfur. In some embodiments, -Cy- is or comprises an optionally substituted bivalent 5-6 membered heteroaryl ring having 1-3 heteroatoms independently selected from oxygen, nitrogen, sulfur. In some embodiments, -Cy- is or comprises an optionally substituted bivalent 5-6 membered heteroaryl ring having 1-2 heteroatoms independently selected from oxygen, nitrogen, sulfur. In some embodiments, -Cy- is or comprises an optionally substituted bivalent 5-6 membered heteroaryl ring having one heteroatom independently selected from oxygen, nitrogen, sulfur. In some embodiments, -Cy-H is optionally substituted heteroaryl as described in the present disclosure, for example, heteroaryl embodiments for R. In some embodiments, -Cy- is

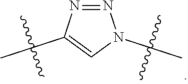 [SMILES: *c1cn(*)nn1]

In some embodiments, -Cy- is or comprises an optionally substituted bivalent 3-20 membered heterocyclyl ring having 1-10 heteroatoms independently selected from oxygen, nitrogen, sulfur, phosphorus and silicon. In some embodiments, -Cy- is or comprises an optionally substituted bivalent 3-20 membered heterocyclyl ring having 1-10 heteroatoms independently selected from oxygen, nitrogen, and sulfur. In some embodiments, -Cy- is or comprises an optionally substituted bivalent 3-6 membered heterocyclyl ring having 1-4 heteroatoms independently selected from oxygen, nitrogen, sulfur. In some embodiments, -Cy- is or comprises an optionally substituted bivalent 5-6 membered heterocyclyl ring having 1-4 heteroatoms independently selected from oxygen, nitrogen, sulfur. In some embodiments, -Cy- is or comprises an optionally substituted bivalent 5-6 membered heterocyclyl ring having 1-3 heteroatoms independently selected from oxygen, nitrogen, sulfur. In some embodiments, -Cy- is or comprises an optionally substituted bivalent 5-6 membered heterocyclyl ring having 1-2 heteroatoms independently selected from oxygen, nitrogen, sulfur. In some embodiments, -Cy- is or comprises an optionally substituted bivalent 5-6 membered heterocyclyl ring having one heteroatom independently selected from oxygen, nitrogen, sulfur. In some embodiments, -Cy- is or comprises an optionally substituted saturated bivalent heterocyclyl group. In some embodiments, -Cy- is or comprises an optionally substituted partially unsaturated bivalent heterocyclyl group. In some embodiments, -Cy-H is optionally substituted heterocyclyl as described in the present disclosure, for example, heterocyclyl embodiments for R.

In some embodiments, -Cy- is

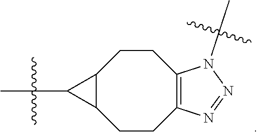 [SMILES: *C1C2CCc3nnn(*)c3CCC12]

In some embodiments, -Cy- is

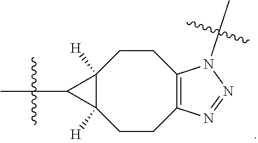 [SMILES: *C1[C@H]2CCc3c(nnn3*)CC[C@@H]12]

In some embodiments, -Cy- is

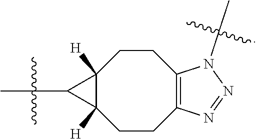 [SMILES: *C1[C@H]2CCc3nnn(*)c3CC[C@@H]12]

In some embodiments, -Cy- is

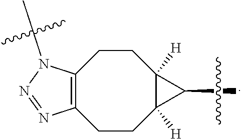

In some embodiments, -Cy- is

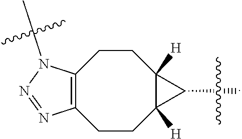

In some embodiments, each Xaa is independently an amino acid residue. In some embodiments, each Xaa is independently an amino acid residue of an amino acid of formula A-I.

In some embodiments, t is 0. In some embodiments, t is 1-50. In some embodiments, t is z as described in the present disclosure.

In some embodiments, y is 1. In some embodiments, y is 2. In some embodiments, y is 3. In some embodiments, y is 4. In some embodiments, y is 5. In some embodiments, y is 6. In some embodiments, y is 7. In some embodiments, y is 8. In some embodiments, y is 9. In some embodiments, y is 10. In some embodiments, y is 11. In some embodiments, y is 12. In some embodiments, y is 13. In some embodiments, y is 14. In some embodiments, y is 15. In some embodiments, y is 16. In some embodiments, y is 17. In some embodiments, y is 18. In some embodiments, y is 19. In some embodiments, y is 20. In some embodiments, y is greater than 20.

In some embodiments, z is 1. In some embodiments, z is 2. In some embodiments, z is 3. In some embodiments, z is 4. In some embodiments, z is 5. In some embodiments, z is 6. In some embodiments, z is 7. In some embodiments, z is 8. In some embodiments, z is 9. In some embodiments, z is 10. In some embodiments, z is 11. In some embodiments, z is 12. In some embodiments, z is 13. In some embodiments, z is 14. In some embodiments, z is 15. In some embodiments, z is 16. In some embodiments, z is 17. In some embodiments, z is 18. In some embodiments, z is 19. In some embodiments, z is 20. In some embodiments, z is greater than 20.

In some embodiments, R^{c} is R′ as described in the present disclosure. In some embodiments, R^{c} is R as described in the present disclosure. In some embodiments, R^{c} is —N(R′)₂, wherein each R′ is independently as described in the present disclosure. In some embodiments, R^{c} is —NH₂. In some embodiments, R^{c} is R—C(O)—, wherein R is as described in the present disclosure. In some embodiments, R^{c} is —H.

In some embodiments, a is 1. In some embodiments, a is 2-100. In some embodiments, a is 5. In some embodiments, a is 10. In some embodiments, a is 20. In some embodiments, a is 50.

In some embodiments, b is 1. In some embodiments, b is 2-100. In some embodiments, b is 5. In some embodiments, b is 10. In some embodiments, b is 20. In some embodiments, b is 50.

In some embodiments, a1 is 0. In some embodiments, a1 is 1.

In some embodiments, a2 is 0. In some embodiments, a2 is 1.

In some embodiments, L^{b} is L^{a} as described in the present disclosure. In some embodiments, L^{b} comprises -Cy-. In some embodiments, L^{b} comprises a double bond. In some embodiments, L^{b} comprises —S—. In some embodiments, L^{b} comprises —S—S—. In some embodiments, L^{b} comprises —C(O)—N(R′)—.

In some embodiments, R′ is —R, —C(O)R, —C(O)OR, or —S(O)₂R, wherein R is as described in the present disclosure. In some embodiments, R′ is R, wherein R is as described in the present disclosure. In some embodiments, R′ is —C(O)R, wherein R is as described in the present disclosure. In some embodiments, R′ is —C(O)OR, wherein R is as described in the present disclosure. In some embodiments, R′ is —S(O)₂R, wherein R is as described in the present disclosure. In some embodiments, R′ is hydrogen. In some embodiments, R′ is not hydrogen. In some embodiments, R′ is R, wherein R is optionally substituted C₁₋₂₀ aliphatic as described in the present disclosure. In some embodiments, R′ is R, wherein R is optionally substituted C₁₋₂₀ heteroaliphatic as described in the present disclosure. In some embodiments, R′ is R, wherein R is optionally substituted C₆₋₂₀ aryl as described in the present disclosure. In some embodiments, R′ is R, wherein R is optionally substituted C₆₋₂₀ arylaliphatic as described in the present disclosure. In some embodiments, R′ is R, wherein R is optionally substituted C₆₋₂₀ arylheteroaliphatic as described in the present disclosure. In some embodiments, R′ is R, wherein R is optionally substituted 5-20 membered heteroaryl as described in the present disclosure. In some embodiments, R′ is R, wherein R is optionally substituted 3-20 membered heterocyclyl as described in the present disclosure. In some embodiments, two or more R′ are R, and are optionally and independently taken together to form an optionally substituted ring as described in the present disclosure.

In some embodiments, each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon.

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, each R is independently —H, or an optionally substituted group selected from C₁₋₂₀ aliphatic, C₁₋₂₀ heteroaliphatic having 1-10 heteroatoms independently selected from oxygen, nitrogen, sulfur, phosphorus and silicon, C₆₋₂₀ aryl, C₆₋₂₀ arylaliphatic, C₆₋₂₀ arylheteroaliphatic having 1-10 heteroatoms independently selected from oxygen, nitrogen, sulfur, phosphorus and silicon, 5-20 membered heteroaryl having 1-10 heteroatoms independently selected from oxygen, nitrogen, sulfur, phosphorus and silicon, and 3-20 membered heterocyclyl having 1-10 heteroatoms independently selected from oxygen, nitrogen, sulfur, phosphorus and silicon, or

two R groups are optionally and independently taken together to form a covalent bond, or:

two or more R groups on the same atom are optionally and independently taken together with the atom to form an optionally substituted, 3-20 membered monocyclic, bicyclic or polycyclic ring having, in addition to the atom, 0-10 heteroatoms independently selected from oxygen, nitrogen, sulfur, phosphorus and silicon.

two or more R groups on two or more atoms are optionally and independently taken together with their intervening atoms to form an optionally substituted, 3-20 membered monocyclic, bicyclic or polycyclic ring having, in addition to the intervening atoms, 0-10 heteroatoms independently selected from oxygen, nitrogen, sulfur, phosphorus and silicon.

In some embodiments, each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon.

In some embodiments, each R is independently —H, or an optionally substituted group selected from C₁₋₂₀ aliphatic, C₁₋₂₀ heteroaliphatic having 1-10 heteroatoms independently selected from oxygen, nitrogen, sulfur, phosphorus and silicon, C₆₋₂₀ aryl, C₆₋₂₀ arylaliphatic, C₆₋₂₀ arylheteroaliphatic having 1-10 heteroatoms independently selected from oxygen, nitrogen, sulfur, phosphorus and silicon, 5-20 membered heteroaryl having 1-10 heteroatoms independently selected from oxygen, nitrogen, sulfur, phosphorus and silicon, and 3-20 membered heterocyclyl having 1-10 heteroatoms independently selected from oxygen, nitrogen, sulfur, phosphorus and silicon.

In some embodiments, R is hydrogen. In some embodiments, R is not hydrogen. In some embodiments, R is an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, a 5-30 membered heteroaryl ring having 1-10 heteroatoms independently selected from oxygen, nitrogen, sulfur, phosphorus and silicon, and a 3-30 membered heterocyclic ring having 1-10 heteroatoms independently selected from oxygen, nitrogen, sulfur, phosphorus and silicon.

In some embodiments, R is hydrogen or an optionally substituted group selected from C₁₋₂₀ aliphatic, phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, and sulfur, a 4-7 membered saturated or partially unsaturated heterocyclic ring having 1-3 heteroatoms independently selected from nitrogen, oxygen, and sulfur, a 7-10 membered bicyclic saturated or partially unsaturated heterocyclic ring having 1-5 heteroatoms independently selected from nitrogen, oxygen, and sulfur, or an 8-10 membered bicyclic heteroaryl ring having 1-5 heteroatoms independently selected from nitrogen, oxygen, and sulfur.

In some embodiments, R is optionally substituted C₁₋₃₀ aliphatic. In some embodiments, R is optionally substituted C₁₋₂₀ aliphatic. In some embodiments, R is optionally substituted C₁₋₁₅ aliphatic. In some embodiments, R is optionally substituted C₁₋₁₀ aliphatic. In some embodiments, R is optionally substituted C₁₋₆ aliphatic. In some embodiments, R is optionally substituted C₁₋₆ alkyl. In some embodiments, R is optionally substituted hexyl, pentyl, butyl, propyl, ethyl or methyl. In some embodiments, R is optionally substituted hexyl. In some embodiments, R is optionally substituted pentyl. In some embodiments, R is optionally substituted butyl. In some embodiments, R is optionally substituted propyl. In some embodiments, R is optionally substituted ethyl. In some embodiments, R is optionally substituted methyl. In some embodiments, R is hexyl. In some embodiments, R is pentyl. In some embodiments, R is butyl. In some embodiments, R is propyl. In some embodiments, R is ethyl. In some embodiments, R is methyl. In some embodiments, R is isopropyl. In some embodiments, R is n-propyl. In some embodiments, R is tert-butyl. In some embodiments, R is sec-butyl. In some embodiments, R is n-butyl. In some embodiments, R is —(CH₂)₂CN.

In some embodiments, R is optionally substituted C₃₋₃₀ cycloaliphatic. In some embodiments, R is optionally substituted C₃₋₂₀ cycloaliphatic. In some embodiments, R is optionally substituted C₃₋₁₀ cycloaliphatic. In some embodiments, R is optionally substituted cyclohexyl. In some embodiments, R is cyclohexyl. In some embodiments, R is optionally substituted cyclopentyl. In some embodiments, R is cyclopentyl. In some embodiments, R is optionally substituted cyclobutyl. In some embodiments, R is cyclobutyl. In some embodiments, R is optionally substituted cyclopropyl. In some embodiments, R is cyclopropyl.

In some embodiments, R is an optionally substituted 3-30 membered saturated or partially unsaturated carbocyclic ring. In some embodiments, R is an optionally substituted 3-7 membered saturated or partially unsaturated carbocyclic ring. In some embodiments, R is an optionally substituted 3-membered saturated or partially unsaturated carbocyclic ring. In some embodiments, R is an optionally substituted 4-membered saturated or partially unsaturated carbocyclic ring. In some embodiments, R is an optionally substituted 5-membered saturated or partially unsaturated carbocyclic ring. In some embodiments, R is an optionally substituted 6-membered saturated or partially unsaturated carbocyclic ring. In some embodiments, R is an optionally substituted 7-membered saturated or partially unsaturated carbocyclic ring. In some embodiments, R is optionally substituted cycloheptyl. In some embodiments, R is cycloheptyl. In some embodiments, R is optionally substituted cyclohexyl. In some embodiments, R is cyclohexyl. In some embodiments, R is optionally substituted cyclopentyl. In some embodiments, R is cyclopentyl. In some embodiments, R is optionally substituted cyclobutyl. In some embodiments, R is cyclobutyl. In some embodiments, R is optionally substituted cyclopropyl. In some embodiments, R is cyclopropyl.

In some embodiments, when R is or comprises a ring structure, e.g., cycloaliphatic, cycloheteroaliphatic, aryl, heteroaryl, etc., the ring structure can be monocyclic, bicyclic or polycyclic. In some embodiments, R is or comprises a monocyclic structure. In some embodiments, R is or comprises a bicyclic structure. In some embodiments, R is or comprises a polycyclic structure.

In some embodiments, R is optionally substituted C₁₋₃₀ heteroaliphatic having 1-10 heteroatoms independently selected from oxygen, nitrogen, sulfur, phosphorus and silicon. In some embodiments, R is optionally substituted C₁₋₂₀ heteroaliphatic having 1-10 heteroatoms. In some embodiments, R is optionally substituted C₁₋₂₀ heteroaliphatic having 1-10 heteroatoms independently selected from oxygen, nitrogen, sulfur, phosphorus or silicon, optionally including one or more oxidized forms of nitrogen, sulfur, phosphorus or selenium. In some embodiments, R is optionally substituted C₁₋₃₀ heteroaliphatic comprising 1-10 groups independently selected from

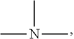

### —N═, ≡N, —S—, —S(O)—, —S(O)₂—, —O—, ═O,

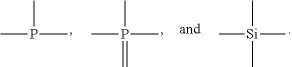

In some embodiments, R is optionally substituted C₆₋₃₀ aryl. In some embodiments, R is optionally substituted phenyl. In some embodiments, R is phenyl. In some embodiments, R is substituted phenyl.

In some embodiments, R is an optionally substituted 8-10 membered bicyclic saturated, partially unsaturated or aryl ring. In some embodiments, R is an optionally substituted 8-10 membered bicyclic saturated ring. In some embodiments, R is an optionally substituted 8-10 membered bicyclic partially unsaturated ring. In some embodiments, R is an optionally substituted 8-10 membered bicyclic aryl ring. In some embodiments, R is optionally substituted naphthyl.

In some embodiments, R is optionally substituted 5-30 membered heteroaryl ring having 1-10 heteroatoms independently selected from oxygen, nitrogen, sulfur, phosphorus and silicon. In some embodiments, R is optionally substituted 5-30 membered heteroaryl ring having 1-10 heteroatoms independently selected from oxygen, nitrogen, and sulfur. In some embodiments, R is optionally substituted 5-30 membered heteroaryl ring having 1-5 heteroatoms independently selected from oxygen, nitrogen, sulfur, phosphorus and silicon. In some embodiments, R is optionally substituted 5-30 membered heteroaryl ring having 1-5 heteroatoms independently selected from oxygen, nitrogen, and sulfur.

In some embodiments, R is an optionally substituted 5-6 membered monocyclic heteroaryl ring having 1-4 heteroatoms independently selected from nitrogen, oxygen, and sulfur. In some embodiments, R is a substituted 5-6 membered monocyclic heteroaryl ring having 1-4 heteroatoms independently selected from nitrogen, oxygen, and sulfur. In some embodiments, R is an unsubstituted 5-6 membered monocyclic heteroaryl ring having 1-4 heteroatoms independently selected from nitrogen, oxygen, and sulfur. In some embodiments, R is an optionally substituted 5-6 membered monocyclic heteroaryl ring having 1-3 heteroatoms independently selected from nitrogen, sulfur, and oxygen. In some embodiments, R is a substituted 5-6 membered monocyclic heteroaryl ring having 1-3 heteroatoms independently selected from nitrogen, oxygen, and sulfur. In some embodiments, R is an unsubstituted 5-6 membered monocyclic heteroaryl ring having 1-3 heteroatoms independently selected from nitrogen, sulfur, and oxygen.

In some embodiments, R is an optionally substituted 5-membered monocyclic heteroaryl ring having 1-4 heteroatoms independently selected from nitrogen, oxygen or sulfur. In some embodiments, R is an optionally substituted 6-membered monocyclic heteroaryl ring having 1-4 heteroatoms independently selected from nitrogen, oxygen, and sulfur.

In some embodiments, R is an optionally substituted 5-membered monocyclic heteroaryl ring having one heteroatom selected from nitrogen, oxygen, and sulfur. In some embodiments, R is optionally substituted pyrrolyl, furanyl, or thienyl.

In some embodiments, R is an optionally substituted 5-membered heteroaryl ring having two heteroatoms independently selected from nitrogen, oxygen, and sulfur. In certain embodiments, R is an optionally substituted 5-membered heteroaryl ring having one nitrogen atom, and an additional heteroatom selected from sulfur or oxygen. In some embodiments, R is an optionally substituted 5-membered heteroaryl ring having three heteroatoms independently selected from nitrogen, oxygen, and sulfur. In some embodiments, R is an optionally substituted 5-membered heteroaryl ring having four heteroatoms independently selected from nitrogen, oxygen, and sulfur.

In some embodiments, R is an optionally substituted 6-membered heteroaryl ring having 1-4 nitrogen atoms. In some embodiments, R is an optionally substituted 6-membered heteroaryl ring having 1-3 nitrogen atoms. In other embodiments, R is an optionally substituted 6-membered heteroaryl ring having 1-2 nitrogen atoms. In some embodiments, R is an optionally substituted 6-membered heteroaryl ring having four nitrogen atoms. In some embodiments, R is an optionally substituted 6-membered heteroaryl ring having three nitrogen atoms. In some embodiments, R is an optionally substituted 6-membered heteroaryl ring having two nitrogen atoms. In certain embodiments, R is an optionally substituted 6-membered heteroaryl ring having one nitrogen atom.

In certain embodiments, R is an optionally substituted 8-10 membered bicyclic heteroaryl ring having 1-4 heteroatoms independently selected from nitrogen, oxygen, and sulfur. In some embodiments, R is an optionally substituted 5,6-fused heteroaryl ring having 1-4 heteroatoms independently selected from nitrogen, oxygen, and sulfur. In certain embodiments, R is an optionally substituted 6,6-fused heteroaryl ring having 1-4 heteroatoms independently selected from nitrogen, oxygen, and sulfur.

In some embodiments, R is 3-30 membered heterocyclic ring having 1-10 heteroatoms independently selected from oxygen, nitrogen, sulfur, phosphorus and silicon. In some embodiments, R is 3-30 membered heterocyclic ring having 1-10 heteroatoms independently selected from oxygen, nitrogen, and sulfur. In some embodiments, R is 3-30 membered heterocyclic ring having 1-5 heteroatoms independently selected from oxygen, nitrogen, sulfur, phosphorus and silicon. In some embodiments, R is 3-30 membered heterocyclic ring having 1-5 heteroatoms independently selected from oxygen, nitrogen, and sulfur.

In some embodiments, R is an optionally substituted 3-7 membered saturated or partially unsaturated heterocyclic ring having 1-3 heteroatoms independently selected from nitrogen, oxygen, and sulfur. In some embodiments, R is a substituted 3-7 membered saturated or partially unsaturated heterocyclic ring having 1-3 heteroatoms independently selected from nitrogen, oxygen, and sulfur. In some embodiments, R is an unsubstituted 3-7 membered saturated or partially unsaturated heterocyclic ring having 1-3 heteroatoms independently selected from nitrogen, oxygen, and sulfur. In certain embodiments, R is an optionally substituted 5-7 membered partially unsaturated monocyclic ring having 1-3 heteroatoms independently selected from nitrogen, oxygen, and sulfur. In certain embodiments, R is an optionally substituted 5-6 membered partially unsaturated monocyclic ring having 1-3 heteroatoms independently selected from nitrogen, oxygen, and sulfur. In certain embodiments, R is an optionally substituted 5-membered partially unsaturated monocyclic ring having 1-3 heteroatoms independently selected from nitrogen, oxygen, and sulfur. In certain embodiments, R is an optionally substituted 6-membered partially unsaturated monocyclic ring having 1-3 heteroatoms independently selected from nitrogen, oxygen, and sulfur. In certain embodiments, R is an optionally substituted 7-membered partially unsaturated monocyclic ring having 1-3 heteroatoms independently selected from nitrogen, oxygen, and sulfur. In some embodiments, R is optionally substituted 3-membered heterocyclic ring having one heteroatom selected from nitrogen, oxygen or sulfur. In some embodiments, R is optionally substituted 4-membered heterocyclic ring having 1-3 heteroatoms independently selected from nitrogen, oxygen, and sulfur. In some embodiments, R is optionally substituted 5-membered heterocyclic ring having 1-3 heteroatoms independently selected from nitrogen, oxygen, and sulfur. In some embodiments, R is optionally substituted 6-membered heterocyclic ring having 1-3 heteroatoms independently selected from nitrogen, oxygen, and sulfur. In some embodiments, R is optionally substituted 7-membered heterocyclic ring having 1-3 heteroatoms independently selected from nitrogen, oxygen, and sulfur.

In some embodiments, R is an optionally substituted 3-membered saturated or partially unsaturated heterocyclic ring having 1-2 heteroatoms independently selected from nitrogen, oxygen, and sulfur. In some embodiments, R is an optionally substituted 4-membered saturated or partially unsaturated heterocyclic ring having 1-2 heteroatoms independently selected from nitrogen, oxygen, and sulfur. In some embodiments, R is an optionally substituted 5-membered saturated or partially unsaturated heterocyclic ring having 1-2 heteroatoms independently selected from nitrogen, oxygen, and sulfur. In some embodiments, R is an optionally substituted 6-membered saturated or partially unsaturated heterocyclic ring having 1-2 heteroatoms independently selected from nitrogen, oxygen, and sulfur. In some embodiments, R is an optionally substituted 7-membered saturated or partially unsaturated heterocyclic ring having 1-2 heteroatoms independently selected from nitrogen, oxygen, and sulfur.

In certain embodiments, R is an optionally substituted 5-6 membered partially unsaturated monocyclic ring having 1-2 heteroatoms independently selected from nitrogen, oxygen, and sulfur. In certain embodiments, R is an optionally substituted tetrahydropyridinyl, dihydrothiazolyl, dihydrooxazolyl, or oxazolinyl group.

In some embodiments, R is an optionally substituted 7-10 membered bicyclic saturated or partially unsaturated heterocyclic ring having 1-5 heteroatoms independently selected from nitrogen, oxygen, and sulfur. In some embodiments, R is optionally substituted indolinyl. In some embodiments, R is optionally substituted isoindolinyl. In some embodiments, R is optionally substituted 1, 2, 3, 4-tetrahydroquinolinyl. In some embodiments, R is optionally substituted 1, 2, 3, 4-tetrahydroisoquinolinyl. In some embodiments, R is an optionally substituted azabicyclo[3.2.1]octanyl.

In some embodiments, R is an optionally substituted 8-10 membered bicyclic heteroaryl ring having 1-5 heteroatoms independently selected from nitrogen, oxygen, and sulfur. In some embodiments, R is an optionally substituted 5,6-fused heteroaryl ring having 1-5 heteroatoms independently selected from nitrogen, oxygen, and sulfur.

In some embodiments, R is optionally substituted C₆₋₃₀ arylaliphatic. In some embodiments, R is optionally substituted C₆₋₂₀ arylaliphatic. In some embodiments, R is optionally substituted C₆₋₁₀ arylaliphatic. In some embodiments, an aryl moiety of the arylaliphatic has 6, 10, or 14 aryl carbon atoms. In some embodiments, an aryl moiety of the arylaliphatic has 6 aryl carbon atoms. In some embodiments, an aryl moiety of the arylaliphatic has 10 aryl carbon atoms. In some embodiments, an aryl moiety of the arylaliphatic has 14 aryl carbon atoms. In some embodiments, an aryl moiety is optionally substituted phenyl.

In some embodiments, R is optionally substituted C₆₋₃₀ arylheteroaliphatic having 1-10 heteroatoms independently selected from oxygen, nitrogen, sulfur, phosphorus and silicon. In some embodiments, R is optionally substituted C₆₋₃₀ arylheteroaliphatic having 1-10 heteroatoms independently selected from oxygen, nitrogen, and sulfur. In some embodiments, R is optionally substituted C₆₋₂₀ arylheteroaliphatic having 1-10 heteroatoms independently selected from oxygen, nitrogen, sulfur, phosphorus and silicon. In some embodiments, R is optionally substituted C₆₋₂₀ arylheteroaliphatic having 1-10 heteroatoms independently selected from oxygen, nitrogen, and sulfur. In some embodiments, R is optionally substituted C₆₋₁₀ arylheteroaliphatic having 1-5 heteroatoms independently selected from oxygen, nitrogen, sulfur, phosphorus and silicon. In some embodiments, R is optionally substituted C₆-10 arylheteroaliphatic having 1-5 heteroatoms independently selected from oxygen, nitrogen, and sulfur.

In some embodiments, two R groups are optionally and independently taken together to form a covalent bond. In some embodiments, —C═O is formed. In some embodiments, —C═C— is formed. In some embodiments, —C≡C— is formed.

In some embodiments, two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon. In some embodiments, two or more R groups on the same atom are optionally and independently taken together with the atom to form an optionally substituted, 3-20 membered monocyclic, bicyclic or polycyclic ring having, in addition to the atom, 0-10 heteroatoms independently selected from oxygen, nitrogen, sulfur, phosphorus and silicon. In some embodiments, two or more R groups on the same atom are optionally and independently taken together with the atom to form an optionally substituted, 3-10 membered monocyclic, bicyclic or polycyclic ring having, in addition to the atom, 0-5 heteroatoms independently selected from oxygen, nitrogen, sulfur, phosphorus and silicon. In some embodiments, two or more R groups on the same atom are optionally and independently taken together with the atom to form an optionally substituted, 3-6 membered monocyclic, bicyclic or polycyclic ring having, in addition to the atom, 0-3 heteroatoms independently selected from oxygen, nitrogen, sulfur, phosphorus and silicon. In some embodiments, two or more R groups on the same atom are optionally and independently taken together with the atom to form an optionally substituted, 3-5 membered monocyclic, bicyclic or polycyclic ring having, in addition to the atom, 0-3 heteroatoms independently selected from oxygen, nitrogen, sulfur, phosphorus and silicon.

In some embodiments, two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon. In some embodiments, two or more R groups on two or more atoms are optionally and independently taken together with their intervening atoms to form an optionally substituted, 3-20 membered monocyclic, bicyclic or polycyclic ring having, in addition to the intervening atoms, 0-10 heteroatoms independently selected from oxygen, nitrogen, sulfur, phosphorus and silicon. In some embodiments, two or more R groups on two or more atoms are optionally and independently taken together with their intervening atoms to form an optionally substituted, 3-10 membered monocyclic, bicyclic or polycyclic ring having, in addition to the intervening atoms, 0-10 heteroatoms independently selected from oxygen, nitrogen, sulfur, phosphorus and silicon. In some embodiments, two or more R groups on two or more atoms are optionally and independently taken together with their intervening atoms to form an optionally substituted, 3-10 membered monocyclic, bicyclic or polycyclic ring having, in addition to the intervening atoms, 0-5 heteroatoms independently selected from oxygen, nitrogen, sulfur, phosphorus and silicon. In some embodiments, two or more R groups on two or more atoms are optionally and independently taken together with their intervening atoms to form an optionally substituted, 3-6 membered monocyclic, bicyclic or polycyclic ring having, in addition to the intervening atoms, 0-3 heteroatoms independently selected from oxygen, nitrogen, sulfur, phosphorus and silicon. In some embodiments, two or more R groups on two or more atoms are optionally and independently taken together with their intervening atoms to form an optionally substituted, 3-5 membered monocyclic, bicyclic or polycyclic ring having, in addition to the intervening atoms, 0-3 heteroatoms independently selected from oxygen, nitrogen, sulfur, phosphorus and silicon.

In some embodiments, heteroatoms in R groups, or in the structures formed by two or more R groups taken together, are selected from oxygen, nitrogen, and sulfur. In some embodiments, a formed ring is 3, 4, 5, 6, 7, 8, 9, 10, 11, 12, 13, 14, 15, 16, 17, 18, 19, or 20-membered. In some embodiments, a formed ring is saturated. In some embodiments, a formed ring is partially saturated. In some embodiments, a formed ring is aromatic. In some embodiments, a formed ring comprises a saturated, partially saturated, or aromatic ring moiety. In some embodiments, a formed ring comprises 5, 6, 7, 8, 9, 10, 11, 12, 13, 14, 15, 16, 17, 18, 19, or 20 aromatic ring atoms. In some embodiments, a formed contains no more than 5, 6, 7, 8, 9, 10, 11, 12, 13, 14, 15, 16, 17, 18, 19, or 20 aromatic ring atoms. In some embodiments, aromatic ring atoms are selected from carbon, nitrogen, oxygen and sulfur.

In some embodiments, a ring formed by two or more R groups (or two or more groups selected from R and variables that can be R) taken together is a C₃₋₃₀ cycloaliphatic, C₆₋₃₀ aryl, 5-30 membered heteroaryl having 1-10 heteroatoms independently selected from oxygen, nitrogen, sulfur, phosphorus and silicon, or 3-30 membered heterocyclyl having 1-10 heteroatoms independently selected from oxygen, nitrogen, sulfur, phosphorus and silicon, ring as described for R, but bivalent or multivalent.

Exemplary compounds are set forth in Table 1, below.

In some embodiments, the present disclosure provides a compound set forth in Table 1, above, or a pharmaceutically acceptable salt thereof. In some embodiments, the present disclosure provides a compound I-1 or a pharmaceutically acceptable salt thereof. In some embodiments, the present disclosure provides a compound I-2 or a pharmaceutically acceptable salt thereof. In some embodiments, the present disclosure provides a compound I-3 or a pharmaceutically acceptable salt thereof. In some embodiments, the present disclosure provides a compound I-4 or a pharmaceutically acceptable salt thereof. In some embodiments, the present disclosure provides a compound I-5 or a pharmaceutically acceptable salt thereof. In some embodiments, the present disclosure provides a compound I-6 or a pharmaceutically acceptable salt thereof. In some embodiments, the present disclosure provides a compound I-7 or a pharmaceutically acceptable salt thereof. In some embodiments, the present disclosure provides a compound I-8 or a pharmaceutically acceptable salt thereof. In some embodiments, the present disclosure provides a compound I-9 or a pharmaceutically acceptable salt thereof. In some embodiments, the present disclosure provides a compound I-10 or a pharmaceutically acceptable salt thereof. In some embodiments, the present disclosure provides a compound I-11 or a pharmaceutically acceptable salt thereof. In some embodiments, the present disclosure provides a compound I-12 or a pharmaceutically acceptable salt thereof. In some embodiments, the present disclosure provides a compound I-13 or a pharmaceutically acceptable salt thereof. In some embodiments, the present disclosure provides a compound I-14 or a pharmaceutically acceptable salt thereof. In some embodiments, the present disclosure provides a compound I-15 or a pharmaceutically acceptable salt thereof. In some embodiments, the present disclosure provides a compound I-16 or a pharmaceutically acceptable salt thereof. In some embodiments, the present disclosure provides a compound I-17 or a pharmaceutically acceptable salt thereof. In some embodiments, the present disclosure provides a compound I-18 or a pharmaceutically acceptable salt thereof. In some embodiments, the present disclosure provides a compound I-19 or a pharmaceutically acceptable salt thereof. In some embodiments, the present disclosure provides a compound I-24 or a pharmaceutically acceptable salt thereof. In some embodiments, the present disclosure provides a compound I-25 or a pharmaceutically acceptable salt thereof. In some embodiments, the present disclosure provides a compound I-26 or a pharmaceutically acceptable salt thereof. In some embodiments, the present disclosure provides a compound I-27 or a pharmaceutically acceptable salt thereof. In some embodiments, the present disclosure provides a compound I-28 or a pharmaceutically acceptable salt thereof. In some embodiments, the present disclosure provides a compound I-29 or a pharmaceutically acceptable salt thereof. In some embodiments, the present disclosure provides a compound I-30 or a pharmaceutically acceptable salt thereof. In some embodiments, the present disclosure provides a compound I-31 or a pharmaceutically acceptable salt thereof. In some embodiments, the present disclosure provides a compound I-32 or a pharmaceutically acceptable salt thereof. In some embodiments, the present disclosure provides a compound I-33 or a pharmaceutically acceptable salt thereof. In some embodiments, the present disclosure provides a compound I-34 or a pharmaceutically acceptable salt thereof. In some embodiments, the present disclosure provides a compound I-35 or a pharmaceutically acceptable salt thereof. In some embodiments, the present disclosure provides a compound I-36 or a pharmaceutically acceptable salt thereof. In some embodiments, the present disclosure provides a compound I-37 or a pharmaceutically acceptable salt thereof. In some embodiments, the present disclosure provides a compound I-38 or a pharmaceutically acceptable salt thereof. In some embodiments, the present disclosure provides a compound I-39 or a pharmaceutically acceptable salt thereof. In some embodiments, the present disclosure provides a compound I-40 or a pharmaceutically acceptable salt thereof. In some embodiments, the present disclosure provides a compound I-41 or a pharmaceutically acceptable salt thereof. In some embodiments, the present disclosure provides a compound I-42 or a pharmaceutically acceptable salt thereof. In some embodiments, the present disclosure provides a compound I-43 or a pharmaceutically acceptable salt thereof. In some embodiments, the present disclosure provides a compound I-44 or a pharmaceutically acceptable salt thereof. In some embodiments, the present disclosure provides a compound I-45 or a pharmaceutically acceptable salt thereof. In some embodiments, the present disclosure provides a compound I-46 or a pharmaceutically acceptable salt thereof. In some embodiments, the present disclosure provides a compound I-47 or a pharmaceutically acceptable salt thereof.

### 4. General Methods of Providing the Present Compounds

Compounds of the present disclosure may be prepared or isolated in general by synthetic and/or semi-synthetic methods known to those skilled in the art for analogous compounds and by methods described in detail in the Examples, herein.

In some embodiments, where a particular protecting group (“PG”), leaving group (“LG”), or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

In some embodiments, leaving groups include but are not limited to, halogens (e.g. fluoride, chloride, bromide, iodide), sulfonates (e.g. mesylate, tosylate, benzenesulfonate, brosylate, nosylate, triflate), diazonium, and the like.

In some embodiments, an oxygen protecting group includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

One of skill in the art will appreciate that compounds of formula I, II or III may contain one or more stereocenters, and may be present as a racemic or diastereomeric mixture. One of skill in the art will also appreciate that there are many methods known in the art for the separation of isomers to obtain stereoenriched or stereopure isomers of those compounds, including but not limited to HPLC, chiral HPLC, fractional crystallization of diastereomeric salts, kinetic enzymatic resolution (e.g. by fungal-, bacterial-, or animal-derived lipases or esterases), and formation of covalent diastereomeric derivatives using an enantioenriched reagent.

One of skill in the art will appreciate that various functional groups present in compounds of the present disclosure such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. “March's Advanced Organic Chemistry”, 5^{th} Ed., Ed.: Smith, M. B. and March, J., John Wiley & Sons, New York: 2001, the entirety of which is incorporated herein by reference. Such interconversions may require one or more of the aforementioned techniques, and certain methods for synthesizing compounds of the present disclosure are described below in the Exemplification.

In some embodiments, the present disclosure provides compounds that are useful for preparing ARMs. In some embodiments, the present disclosure provides compounds that are useful for construction of ARM molecules through cycloaddition reactions, e.g., click chemistry or variants thereof.

In some embodiments, the present disclosure provides a compound having the structure of formula IV:

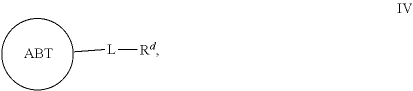

or a salt thereof, wherein

ABT is an antibody binding moiety;

L is a linker moiety;

R^{d} is or comprises a reactive group;

each L is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;

each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;

each R′ is independently —R, —C(O)R, —CO₂R, or —SO₂R;

each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

As appreciated by those skilled in the art and demonstrated herein, various reactive groups may be utilized in accordance with the present disclosure. For example, in some embodiments, R^{d} is -L^{a}-R′, wherein R^{d} comprises —C≡C— or —N₃; such R^{d} are, among other things, useful for click chemistry reactions.

In some embodiments, the present disclosure provides a compound of formula IV-a:

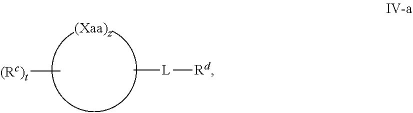

or a salt thereof, wherein each variable is independently as described in the present disclosure.

In some embodiments, the present disclosure provides a compound of formula IV-b:

R^{c}-(Xaa)_{z}-L-R^{d}, IV-b

or a salt thereof, wherein each variable is independently as described in the present disclosure.

In some embodiments, the present disclosure provides a compound of formula IV-c:

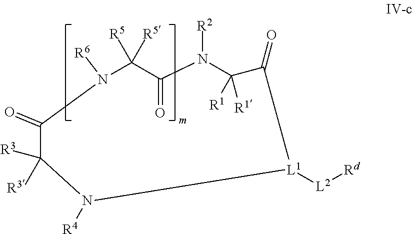

or a salt thereof, wherein each variable is independently as described in the present disclosure.

In some embodiments, the present disclosure provides a compound of formula IV-d:

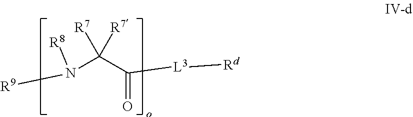

or a salt thereof, wherein each variable is independently as described in the present disclosure.

In some embodiments, the present disclosure provides a compound of formula V:

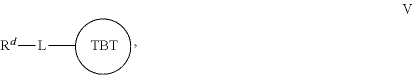

or a salt thereof, wherein each variable is independently as described in the present disclosure.

In some embodiments,

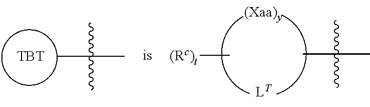

is L as described herein. In some embodiments, a target binding moiety binds to CD38.

In some embodiments, the present disclosure provides a method, comprising:

a) providing a first compound comprising a target binding moiety as described herein and a first reactive group;

b) providing a second compound comprising an antibody binding moiety as described herein and a second reactive group; and

c) reacting the first reactive group with the second reactive group such that the target binding moiety and the antibody binding moiety are covalent linked.

In some embodiments, a first compound is of formula IV, IV-a, IV-b, IV-c, or IV-d, or a salt thereof. In some embodiments, a second compound is of formula V or a salt thereof.

In some embodiments, the present disclosure provides a method for preparing a compound, comprising steps of:

providing a first compound of formula IV, IV-a, IV-b, IV-c, or IV-d, or a salt thereof, wherein the compound comprising a first reactive moiety;

providing a second compound of formula V comprising a second reactive moiety or a salt thereof; and

reacting the first compound with the second compound, wherein the first reactive moiety reacts with the second reaction moiety to link a first compound and a second compound.

Various reactive moieties and reactions can be utilized in accordance with the present disclosure. For example, in some embodiments, a reaction is an amidate reaction, wherein one of a first reactive moiety (e.g., R^{d}) and a second reactive moiety (e.g. R^{d}) is or comprises an activated carboxylic acid (e.g., is or comprises

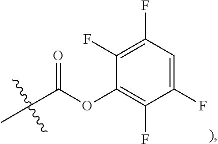 [SMILES: *C(=O)Oc1c(F)c(F)cc(F)c1F]

and the other is or comprises an amino group.

In some embodiments, the present disclosure provides a method for preparing a compound, comprising steps of:

providing a first compound of formula IV, IV-a, IV-b, IV-c, or IV-d, or a salt thereof, wherein the compound comprising a first reactive moiety;

providing a second compound of formula V comprising a second reactive moiety or a salt thereof; and

reacting the first compound with the second compound, wherein the first reactive moiety reacts with the second reaction moiety through a cycloaddition reaction.

Many cycloaddition reactions can be utilized in accordance with the present disclosure. In some embodiments, a cycloaddition reaction is a [4+2] reaction. In some embodiments, a cycloaddition reaction is a [3+2] reaction. In some embodiments, a [3+2] reaction is a click chemistry reaction. In some embodiments, a first reactive moiety is —C≡C— and the second reactive moiety is —N₃. In some embodiments, a first reactive moiety is —N₃ and the second reactive moiety is —C≡C—.

In some embodiments, provided technologies (e.g., compounds, agents, etc.) is or comprises a peptide moiety. Those skilled in the art appreciate that variable peptide synthesis technologies are readily available and can be utilized in accordance with the present disclosure.

Various technologies are available and can be utilized to assess provided technologies, e.g., properties and/or activities of provided compounds (e.g., CD38 binding, recruitment of immune activities, ADCC, etc.), in accordance with the present disclosure. Certain useful technologies are described in the Examples.

### 5. Uses, Formulation and Administration

Pharmaceutically Acceptable Compositions

According to another embodiment, present disclosure provides a composition comprising a compound described herein or a pharmaceutically acceptable derivative thereof and a pharmaceutically acceptable carrier, adjuvant, or vehicle. In some embodiments, the present disclosure provides a pharmaceutical composition comprising a compound, e.g., an ARM, of the present disclosure and a pharmaceutically acceptable carrier. In some embodiments, the present disclosure provides a pharmaceutical composition comprising a therapeutically effective amount of a compound, e.g., an ARM, of the present disclosure and a pharmaceutically acceptable carrier. In some embodiments, an amount of a compound in a composition is such that it is effective to direct antibodies selectively to targets, e.g., diseased cells (e.g., cancer cells), and/or induce antibody-directed activities, e.g., cell-mediated immunity such as cytotoxicity. In certain embodiments, an amount of a compound in a composition is such that is effective to direct antibodies selectively to cancer cells expressing CD38, and induce antibody-directed activities, e.g., cell-mediated cytotoxicity, in a biological sample or in a subject (e.g., a cancer patient). In certain embodiments, a composition is formulated for administration to a patient in need of such composition. In some embodiments, a composition is formulated for oral administration to a patient.

In some embodiments, a pharmaceutically acceptable carrier, adjuvant, or vehicle is a non-toxic carrier, adjuvant, or vehicle that does not destroy the pharmacological activity of the compound with which it is formulated. Pharmaceutically acceptable carriers, adjuvants or vehicles that may be include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In some embodiments, a pharmaceutically acceptable derivative is a non-toxic salt, ester, salt of an ester or other derivative of a compound that, upon administration to a recipient, is capable of providing, either directly or indirectly, a compound or an active metabolite or residue thereof.

Compositions may be administered orally, parenterally, by inhalation spray, topically, rectally, nasally, buccally, vaginally or via an implanted reservoir. In some embodiments, parenteral administration includes subcutaneous, intravenous, intramuscular, intra-articular, intra-synovial, intrasternal, intrathecal, intrahepatic, intralesional and intracranial injection or infusion techniques. In some embodiments, compositions are administered orally, intraperitoneally or intravenously. Sterile injectable forms of compositions may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

In some embodiments, a bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

In some embodiments, pharmaceutically acceptable compositions may be administered in the form of suppositories for rectal administration. In some embodiments, these can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

In some embodiments, pharmaceutically acceptable compositions may be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, pharmaceutically acceptable compositions are formulated for oral administration. Such formulations may be administered with or without food. In some embodiments, pharmaceutically acceptable compositions are administered without food. In other embodiments, pharmaceutically acceptable compositions are administered with food.

Amounts of compounds that may be combined with the carrier materials to produce a composition in a single dosage form will vary depending upon the host treated, the particular mode of administration. In some embodiments, provided compositions are formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

Uses of Compounds and Pharmaceutically Acceptable Compositions

Compounds and compositions described herein are generally useful to direct antibodies selectively to targets, particularly those expressing or comprising CD38, such as diseased cells (e.g., cancer cells), and/or to induce antibody-directed activities, e.g., cell-mediated immune response (e.g., cytotoxicity.)

In some embodiments, the present disclosure provides methods for recruiting antibodies, e.g., endogenous antibodies, to a target comprising contacting the target with a provided agent, compound or composition. In some embodiments, recruited antibodies comprise one or more endogenous antibodies. In some embodiments, recruited antibodies have specificity toward one or more antigens. In some embodiments, recruited antibodies have specificity toward one or more peptide antigens or proteins. In some embodiments, recruited antibodies are heterogeneous in that they are not antibodies toward the same antigen or protein.

In some embodiments, the present disclosure provides methods for recruiting an immune cell to a target, comprising contacting a target with a provided agent, compound or composition.

In some embodiments, the present disclosure provides methods for triggering, generating, encouraging, and/or enhancing one or more immune system activities toward a target, comprising contacting a target with a provided agent, compound or composition. In some embodiments, an immune system activity is or comprises ADCC. In some embodiments, an immune system activity is or comprises ADCP. In some embodiments, an immune system activity is or comprises both ADCC and ADCP. In some embodiments, an immune system activity is or comprises complement dependent cytotoxicity (CDC). In some embodiments, an immune system activity is or comprises ADCVI.

In some embodiments, a target is a cancer cell. In some embodiments, a target is a cancer cells in a subject. In some embodiments, provided methods comprise administering a provided agent, compound or composition to a subject.

In some embodiments, when contacted with its target, provided agents and compounds form complexes with antibodies and Fc receptors on target cells. In some embodiments, the present disclosure provides a complex comprising:

an agent comprising:
an antibody binding moiety,
a target binding moiety, and
optionally a linker moiety,

an Fc region, and

an Fc receptor.

In some embodiments, an antibody binding moiety is a universal antibody binding moiety. In some embodiments, a target binding moiety can bind to CD38.

In some embodiments, the present disclosure provides a complexes comprising two or more complexes each independently comprising:

an agent comprising:
an antibody binding moiety,
a target binding moiety, and
optionally a linker moiety,

an Fc region, and

an Fc receptor,

wherein Fc regions of the complexes are of antibodies and/or fragments thereof toward different antigens or proteins.

In some embodiments, Fc regions of the complexes are of antibodies and/or fragments thereof toward different proteins. In some embodiments, one or more Fc regions are of endogenous antibodies and/or fragments thereof.

In some embodiments, the present disclosure provides a method for treating one or more disorders, diseases, and/or conditions wherein the disorder, disease, or condition is a cancer.

In some embodiments, “neoplasia” or “cancer” is or comprises a pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Representative cancers include, for example, prostate cancer, metastatic prostate cancer, stomach, colon, rectal, liver, pancreatic, lung, breast, cervix uteri, corpus uteri, ovary, testis, bladder, renal, brain/CNS, head and neck, throat, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others, which may be treated by one or more compounds according to the present disclosure. Among other things, provided technologies (e.g., compounds, compositions, methods, etc.) are particularly useful for preventing and/or treating cancer.

Furthermore, the present disclosure provides the use of a compound according to the definitions herein, or a pharmaceutically acceptable salt, or a hydrate or solvate thereof for the preparation of a medicament for the treatment of a proliferative disease.

In some embodiments, the present disclosure provides technologies, e.g., agents, compounds (e.g., ARMs), compositions, methods, etc. that are particularly useful for treating CD38-associated cancers. In some embodiments, provided technologies are particularly useful for treating cancers wherein the cancer cells express or comprise CD38.

Combination Therapies

In some embodiments, provided technologies are administered together with one or more additional therapeutic agents and/or technologies (e.g., for cancer, one or more of additional therapeutic agents, surgery, radiotherapy, etc.). In some embodiments, useful additional therapeutic agents and/or technologies for combination are those that have been utilized to treat that condition, disorder or disease. In certain embodiments, a provided compound, e.g., an ARM, or composition thereof, is administered in combination with another therapeutic agent.

Examples of agents for combination include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, a provided compound or composition thereof is utilized in combination with a monoclonal antibody or an siRNA therapeutic.

An additional agent may be administered separately from a provided compound or composition. Alternatively, additional agents may be part of a single dosage form, mixed together with a provided compound in a single composition. If administered as part of a multiple dosage regime, active agents may be administered simultaneously, sequentially or within a period of time from one another, e.g., within five hours from one another.

A combination therapy may comprise simultaneous or sequential administration of therapeutic agents in accordance with the present disclosure. For example, a combination may be administered simultaneously or sequentially in separate unit dosage forms or together in a single unit dosage form.

In some embodiments, amounts of additional therapeutic agents are no more than those that would be administered when the additional therapeutic agents are not combined with compounds or compositions provided herein.

In one embodiment, the present disclosure provides a composition comprising a compound of formula I, II or III and one or more additional therapeutic agents. The therapeutic agent may be administered together with a compound of formula I, II or III, or may be administered prior to or following administration of a compound of formula I, II or III. Suitable therapeutic agents are described in further detail below. In certain embodiments, a compound of formula I, II or III may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a compound of formula I, II or III may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In some embodiments, the present disclosure provides a method of treating an inflammatory disease, disorder or condition by administering to a patient in need thereof a compound of formula I, II or III and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron ®) in combination with lenalidomide (Revlimid®), or any combination(s) thereof.

In some embodiments, the present disclosure provides a method of treating gout comprising administering to a patient in need thereof a compound of formula I, II or III and one or more additional therapeutic agents selected from non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol and febuxostat (Uloric®).

In some embodiments, the present disclosure provides a method of treating rheumatoid arthritis comprising administering to a patient in need thereof a compound of formula I, II or III and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, the present disclosure provides a method of treating osteoarthritis comprising administering to a patient in need thereof a compound of formula I, II or III and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, the present disclosure provides a method of treating lupus comprising administering to a patient in need thereof a compound of formula I, II or III and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, the present disclosure provides a method of treating inflammatory bowel disease comprising administering to a patient in need thereof a compound of formula I, II or III and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, the present disclosure provides a method of treating asthma comprising administering to a patient in need thereof a compound of formula I, II or III and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (Theo-Dur®, Theolair®, Slo-bid®, Uniphyl®, Theo-24®) and aminophylline, and IgE antibodies such as omalizumab (Xolair®).

In some embodiments, the present disclosure provides a method of treating COPD comprising administering to a patient in need thereof a compound of formula I, II or III and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-bid®, Uniphyl®, Theo-24®) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®,

In some embodiments, the present disclosure provides a method of treating HIV comprising administering to a patient in need thereof a compound of formula I, II or III and one or more additional therapeutic agents selected from nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), and combinations thereof.

In some embodiments, the present disclosure provides a method of treating cancer comprising administering to a subject in need thereof a compound of formula I, II or III and one or more additional therapeutic agents. In some embodiments, a cancer is multiple myeloma. In some embodiments, an additional therapeutic agent is lenalidomide. In some embodiments, an additional therapeutic agent is bortezomib. In some embodiments, an additional therapeutic agent is dexamethasone. In some embodiments, an additional therapeutic agent is melphalan. In some embodiments, an additional therapeutic agent is prednisone. In some embodiments, a combination is a provided compound, e.g., an ARM, in combination with lenalidomide or bortezomib and dexamethasone. In some embodiments, a combination is a provided compound, e.g., an ARM, in combination with bortezomib, melphalan and prednisone.

In some embodiments, as demonstrated herein, provided compounds, e.g., ARMs, have low toxicity compared to CD38 antibody therapeutics (e.g., less complement activation, less reduction of CD38-expressing effector cells, etc.). In some embodiments, provided compounds or compositions thereof can be administered at higher and/or more frequent dosages and/or for longer periods of time, either as monotherapies or as part of combination therapies, compared to other therapies such as CD38 antibody therapeutics.

In some embodiments, the present disclosure provides a method of treating a hematological malignancy comprising administering to a patient in need thereof a compound of formula I, II or III and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In some embodiments, the present disclosure provides a method of treating a solid tumor comprising administering to a patient in need thereof a compound of formula I, II or III and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In some embodiments, the present disclosure provides a method of treating a hematological malignancy comprising administering to a patient in need thereof a compound of formula I, II or III and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17, and incorporated herein by reference in its entirety).

In some embodiments, the present disclosure provides a method of treating diffuse large B-cell lymphoma (DLBCL) comprising administering to a patient in need thereof a compound of formula I, II or III and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In some embodiments, the present disclosure provides a method of treating multiple myeloma comprising administering to a patient in need thereof a compound of formula I, II or III and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In some embodiments, the present disclosure provides a method of treating Waldenström's macroglobulinemia comprising administering to a patient in need thereof a compound of formula I, II or III and one or more additional therapeutic agents selected from chlorambucil (Leukeran®), cyclophosphamide (Cytoxan®, Neosar®), fludarabine (Fludara®), cladribine (Leustatin®), rituximab (Rituxan®), a hedgehog signaling inhibitor, a BTK inhibitor, a JAK/pan-JAK inhibitor, a TYK2 inhibitor, a PI3K inhibitor, and a SYK inhibitor.

In some embodiments, the present disclosure provides a method of treating Alzheimer's disease comprising administering to a patient in need thereof a compound of formula I, II or III and one or more additional therapeutic agents selected from donepezil (Aricept®), rivastigmine (Excelon®), galantamine (Razadyne®), tacrine (Cognex®), and memantine (Namenda®).

In some embodiments, the present disclosure provides a method of treating organ transplant rejection or graft vs. host disease comprising administering to a patient in need thereof a compound of formula I, II or III and one or more additional therapeutic agents selected from a steroid, cyclosporin, FK506, rapamycin, a hedgehog signaling inhibitor, a BTK inhibitor, a JAK/pan-JAK inhibitor, a TYK2 inhibitor, a PI3K inhibitor, and a SYK inhibitor.

In some embodiments, the present disclosure provides a method of treating or lessening the severity of a disease comprising administering to a patient in need thereof a compound of formula I, II or III and a BTK inhibitor, wherein the disease is selected from inflammatory bowel disease, arthritis, systemic lupus erythematosus (SLE), vasculitis, idiopathic thrombocytopenic purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., mastocytoma, mast cell leukemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, diabetes, septic shock, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituitarism, Guillain-Barre syndrome, Behcet's disease, scleraderma, mycosis fungoides, acute inflammatory responses (such as acute respiratory distress syndrome and ischemia/reperfusion injury), and Graves' disease.

In some embodiments, the present disclosure provides a method of treating or lessening the severity of a disease comprising administering to a patient in need thereof a compound of formula I, II or III and a PI3K inhibitor, wherein the disease is selected from a cancer, a neurodegenerative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In some embodiments, the present disclosure provides a method of treating or lessening the severity of a disease comprising administering to a patient in need thereof a compound of formula I, II or III and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

Compounds and compositions of the present disclosure, may be administered using any amount and any route of administration effective for treating or lessening the severity of a cancer or a proliferative disorder. Exact amounts required may vary from subject to subject, depending on the species, age, and general condition of a subject, severity, a particular agent, a mode of administration, and the like. In some embodiments, compounds are formulated in dosage unit form for ease of administration and uniformity of dosage. In some embodiments, dosage unit form refers to a physically discrete unit of agent appropriate for the patient to be treated. It will be understood, however, that the total daily usage of compounds and compositions will be decided by attending physicians within the scope of sound medical judgment. Specific effective dose levels for any particular patient or organism may depend upon a variety of factors including the condition, disorder or disease being treated and its severity; activity and/or propert of a specific compound employed; a specific composition employed; age, body weight, general health, sex and diet of a subject; time of administration, route of administration, and/or rate of excretion of a specific compound employed; duration of the treatment; drugs used in combination or coincidental with a specific compound employed, and like factors well known in the medical arts. In some embodiments, a patient is an animal, preferably a mammal, and most preferably a human.

Pharmaceutically acceptable compositions can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, compounds may be administered orally or parenterally at dosage levels of about 0.01 mg/kg to about 50 mg/kg and in some embodiments, from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are in some embodiments suppositories which can be prepared by mixing the compounds with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

Active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound include ointments, pastes, creams, lotions, gels, powders, solutions, sprays, inhalants or patches. The active component is admixed under sterile conditions with a pharmaceutically acceptable carrier and any needed preservatives or buffers as may be required. In some embodiments, a composition is an ophthalmic formulation, ear drops, or eye drops. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In some embodiments, the present disclosure provides a method of inhibiting protein kinase activity in a biological sample comprising the step of contacting said biological sample with a compound of the present disclosure, or a composition comprising said compound.

In some embodiments, a biological sample includes, without limitation, cell cultures or extracts thereof; biopsied material obtained from a mammal or extracts thereof; and blood, saliva, urine, feces, semen, tears, or other body fluids or extracts thereof.

Depending upon the particular condition, or disease, to be treated, additional therapeutic agents that are normally administered to treat that condition, may also be present in the compositions of the present disclosure. As used herein, additional therapeutic agents that are normally administered to treat a particular disease, or condition, are known as “appropriate for the disease, or condition, being treated.”

Among other things, compounds and/or compositions of the present disclosure can be employed in combination therapies, that is, compounds and/or compositions of the present disclosure can be administered concurrently with, prior to, or subsequent to, one or more other therapeutic agents or medical procedures, particularly for treatment of various cancers. In some embodiments, a compound of the present disclosure may also be used to advantage in combination with other antiproliferative compounds. The particular combination of therapies (therapeutics or procedures) to employ in a combination regimen will take into account compatibility of a desired other therapeutics and/or procedures and the desired therapeutic effect to be achieved. It will also be appreciated that the therapies employed may achieve a desired effect for the same disorder (for example, a provided compound may be administered concurrently with another anticancer agent), or they may achieve different effects (e.g., control of any adverse effects). In some embodiments, a therapeutic agent is a chemotherapeutic agent or antiproliferative compounds. Exemplary chemotherapy agents include but are not limited to alkylating agents, nitrosourea agents, antimetabolites, antitumor antibiotics, alkaloids derived from plant, topoisomerase inhibitors, hormone therapy medicines, hormone antagonists, aromatase inhibitors, P-glycoprotein inhibitors, platinum complex derivatives, other immunotherapeutic drugs, and other anticancer agents. Further, provided technologies can be used together with hypoleukocytosis (neutrophil) medicines that are cancer treatment adjuvant, thrombopenia medicines, antiemetic drugs, and cancer pain medicines for patient's QOL recovery or be made as a mixture with them. In some embodiments, a therapeutic reagent is an antibody. In some embodiments, a therapeutic agent is an immunomodulatory agent. In some embodiments, an immunomodulatory agent targets cell surface signaling molecules on immune cells. In some embodiments, an immunomodulatory agent targets cell surface signaling molecules on immune cells, wherein the agent is an antagonist blocking a co-inhibitory pathway. In some embodiments, an immunomodulatory agent is a checkpoint blockage agent. In some embodiments, an immunomodulatory agent is an antibody targeting a cell surface signaling protein expressed by immune cells. In some embodiments, an immunomodulatory agent is an antibody targeting a protein selected from PD-1, PD-L1, CTLA4, TIGIT, BTLA, TIM-3, LAG3, B7-H3, and B7-H4. In some embodiments, an immunomodulatory agent is a PD-1 antibody (e.g., nivolumab, pembrolizumab, pidilizumab, BMS 936559, MPDL3280A, etc). In some embodiments, an immunomodulatory agent is a PD-L1 antibody. In some embodiments, an immunomodulatory agent is a CTLA4 antibody (e.g., ipilimumab). In some embodiments, an immunomodulatory agent is a TIGIT antibody. In some embodiments, an immunomodulatory agent is a BTLA antibody. In some embodiments, an immunomodulatory agent is a Tim-3 antibody. In some embodiments, an immunomodulatory agent is a LAG3 antibody. In some embodiments, an immunomodulatory agent is a B7-H3 antibody. In some embodiments, an immunomodulatory agent is a B7-H4 antibody. In some embodiments, an immunomodulatory agent targets cell surface signaling molecules on immune cells, wherein the agent is an agonist engaging a co-stimulatory pathway. In some embodiments, such an immunomodulatory agent is or comprises an antibody targeting a co-stimulatory receptor. In some embodiments, an antibody activates a T cell co-stimulatory receptor. In some embodiments, an antibody targets a member of the tumor necrosis factor (TNF) receptor superfamily. In some embodiments, an antibody targets a protein selected from CD137 (4-1BB), CD357 (GITR, TNFRS18, AITR), CD134 (OX40) and CD 40 (TNFRSF5). In some embodiments, an antibody is an anti-CD137 antibody (e.g., urelumab). In some embodiments, an antibody is an anti-CD357 antibody. In some embodiments, an antibody is an anti-CD40 antibody. In some embodiments, an antibody is an anti-CD134 antibody. Additional exemplary T cell co-stimulatory and co-inhibitory receptors are described in Chen L, Flies DB., Molecular mechanisms of T cell co-stimulation and co-inhibition. Nat. Rev. Immunol. 2013, 13(4), 227-42, and Yao S, et al., Advances in targeting cell surface signalling molecules for immune modulation. Nat. Rev. Drug Discov. 2013, 12(2), 136-40. In some embodiments, a therapeutic agent is an antibodies activating such a stimulatory receptor, or blocking such an inhibitory receptor.

In some embodiments, one or more other therapeutic agents are or comprise tumor-specific immune cells. In some embodiments, one or more other therapeutic agents are or comprise tumor-specific T cells. In some embodiments, one or more other therapeutic agents are or comprise tumor-infiltrating lymphocytes (TILs). In some embodiments, one or more other therapeutic agents are or comprise T cells ectopically expressing a known anti-tumor T cell receptor (TCR). In some embodiments, one or more other therapeutic agents are or comprise chimeric antigen receptors (CAR) T cells. In some embodiments, a provided composition comprises an immunopotentiative substance. Exemplary immunopotentiative substances that can be used in combination with provided compounds, compositions and/or methods include but are not limited to various cytokines and tumor antigens. Cytokines that stimulate immune reactions include, for example, GM-CSF, M-CSF, G-CSF, interferon-α, β, γ, IL-1, IL-2, IL-3, and IL-12, etc. Antibodies to block inhibitory receptors and/or to activate stimulatory receptors, which are widely known in the art and described herein, for example but not limited to, B7 ligand derivatives, anti-CD3 antibodies, anti-CD28 antibodies, and anti-CTLA-4 antibodies can also improve the immune reactions. In some embodiments, a therapeutic agent is a small molecule for immune modulation. In some embodiments, a therapeutic agent is a small molecule that mediating anti-tumor immune activity. In some embodiments, a therapeutic agent is a small molecule that targets an enzyme directly involved in immune regulation. In some embodiments, a therapeutic agent is an indoleamine 2,3-dioxygenase (IDO) inhibitor. In some embodiments, a therapeutic agent is an IDO1 inhibitor, e.g., F001287, indoximod, NLG-919 and INCB024360. In some embodiments, a therapeutic agent is a tryptophan-2,3 dioxygenase (TDO) inhibitor. In some embodiments, a therapeutic agent is an IDO/TDO dual inhibitor. In some embodiments, a therapeutic agent is an IDO-selective inhibitor. In some embodiments, some other embodiments, a therapeutic agent is a TDO-selective inhibitor. In some embodiments, a provided composition comprises an IDO inhibitor and a first construct. In some embodiments, a provided composition comprises an IDO inhibitor, a first construct and a second construct. It is recognized that immune response to a first construct and/or a second construct can be significantly enhanced by administration of an IDO inhibitor.

In some embodiments, a medical procedure that may be used in combination with compounds, compositions and methods of the present application include but are not limited to surgery, radiotherapy (gamma-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF), to name a few), hyperthermia, cryotherapy, and adoptive T-cell transfer (e.g., TIL therapy, transgenic TCRs, CAR T-cell therapy, NK cellular therapy, etc.). In some embodiments, a medical procedure is surgery. In some embodiments, a medical procedure is radiotherapy.

In some embodiments, a provided technology comprises a provided agent (e.g., those comprising moieties that can bind to CD38) and a population of cells (e.g., cells of a cell-based therapy). In some embodiments, a provided technology comprises a provided agent and a population of effector cells. In some embodiments, cell populations are manipulated cell populations, e.g., are typically engineered, activated, enriched and/or expanded, etc. In some embodiments, cell populations are manufactured cell populations. In some embodiments, cell populations are enriched for certain types of desired cells. In some embodiments, effector cells are NKT cells. In some embodiments, effector cells are monocytes. In some embodiments, effector cells are macrophages. In some embodiments, effector cells are T cells. In some embodiments, effector cells are CAR T cells. In some embodiments, effector cells are NK cells, e.g., cytokine-induced memory like NK cells, CAR-NK cells, engineered NK cells expressing or not expressing certain proteins (e.g., receptors), and may be from various sources. As appreciated by those skilled in the art, useful immune cells such as NK cells may be from various sources and/or be engineered in a number of ways. For example, in some embodiments, NK cells are memory like NK cells. In some embodiments, NK cells are cytokine-induced memory like NK cells. In some embodiments, NK cells are derived from stem cells. In some embodiments, NK cells are derived from iPSC lines. In some embodiments, NK cells are derived from a clonal master iPSC line. In some embodiments, NK cells are engineered to express certain receptors, e.g., a high-affinity, optionally non-cleavable CD16 receptor. In some embodiments, NK cells are engineered to express chimeric antigen receptors (CARs), e.g., in some embodiments, NK cells may be engineered to express anti-CD19 CAR. In some embodiments, NK cells are CAR-NK cells. In some embodiments, NK cells are engineered to express cytokine receptor. In some embodiments, NK cells comprise a IL-15 receptor fusion that enhances the persistence and expansion capabilities without requiring co-administration of cytokine support. In some embodiments, NK cells are engineered to prevent expression of certain cell proteins, e.g., certain cell surface proteins. In some embodiments, NK cells are engineered to prevent expression of CD38. In some embodiments, NK cells are derived from placenta. In some embodiments, NK cells are donor NK cells. In some embodiments, NK cells are haploidentical donor NK cells. In some embodiments, NK cells are mismatched donor NK cells. In some embodiments, NK cells are related donor NK cells, e.g., mismatched related donor NK cells. In some embodiments, NK cells are unrelated donor NK cells. In some embodiments, NK cells are derived from a subject, e.g., a patient. In some embodiments, provided technologies comprise an innate cell engager, e.g., an innate cell engager binding to innate cells (e.g., NK cells and macrophages) while binding simultaneously to specific tumor cells. In some embodiments, NK cells are derived from cord blood stem and progenitor cells. In some embodiments, NK cells are derived with modulation of a signaling pathway, e.g., the Notch signaling pathway. In some embodiments, nanoparticles are utilized to improve and/or sustain growth of NK cells. In some embodiments, as described herein, NK cells are generated ex vivo. In some embodiments, NK cells may be cryopreserved and stored in multiple doses as off-the-shelf cell therapy. Examples of certain such technologies include those utilized by Fate Therapeutics, NantKwest Inc., Celularity, Inc., GC Pharma, Sorrento Therapeutics, Inc., Affimed GmbH/MD Anderson Cancer Center, Gamida Cell Ltd., Nohla Therapeutics, Kiadis Pharma N.V., etc. Those skilled in the art will appreciate that, which they can be optionally utilized, antibodies and/or CARs toward specific antigens utilized in certain such technologies may not be required in provided technologies comprising ARMs as described herein. In some embodiments, provided agents, cells (e.g., effector cells), and optional immunoglobulins (e.g., IgG (e.g., intravenous immunoglobulin) are administered to a subject so that the subject can be exposed to the provided agents and cells. In some embodiments, provided agents and cells (e.g., effector cells) are administered concurrently, either in a single composition (e.g., a composition comprising a provided agent, cells (e.g., effector cells), and optionally immunoglobulins) or separately; in some embodiments, provided agents are administered prior or subsequently to cells (e.g., effector cells). In some embodiments, the present disclosure provides methods for treating various conditions, disorders or diseases, e.g., various cancers, comprising administering to a subject suffering therefrom or susceptible thereto provided agents and cells. In some embodiments, cells (e.g., effector cells) are stored (e.g., cryopreserved) together with a provide agent (e.g., an ARM agent). In some embodiments, they are thawed and administered as a single composition. In some embodiments, cells (e.g., effector cells) are administered at one or more doses followed by a dose of a provided agent (e.g., an ARM agent). In some embodiments, a dose of a provided agent is a single systemic dose. In some embodiments, cells (e.g., effector cells) are administered at multiple doses followed by a dose, in some instance a single systemic dose, of a provided agent (e.g., an ARM agent). In some embodiments, an administration (e.g., a systemic administration, in some instances, a single systemic administration) of an agent (e.g., an ARM agent) is followed by one or more, in some instances, multiple, doses of cells (e.g., effector cells). In some embodiments, an administration (e.g., a systemic administration, in some instances, a single systemic administration) of an agent (e.g., an ARM agent) is followed by one or more, in some instances, multiple, doses each of which is independently of cells (e.g., effector cells) or of cells and a provided agent (e.g., an ARM agent), wherein cells and provided agent may be administered as a combined composition or as separate compositions.

Among other things, provided such technologies can provided enhanced efficacy and/or reduced side effects.

Antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin. The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™ Letrozole is marketed under the trade names Femara™ or Femar™. Aminoglutethimide is marketed under the trade name Orimeten™. A combination of the present disclosure comprising a chemotherapeutic agent which is an aromatase inhibitor is particularly useful for the treatment of hormone receptor positive tumors, such as breast tumors.

In some embodiments, antiestrogen is a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination of the present disclosure comprising a chemotherapeutic agent which is an antiestrogen is particularly useful for the treatment of estrogen receptor positive tumors, such as breast tumors.

In some embodiments, anti-androgen is a substance which is capable of inhibiting the biological effects of androgenic hormones and includes, but is not limited to, bicalutamide (Casodex™) The term “gonadorelin agonist” as used herein includes, but is not limited to abarelix, goserelin and goserelin acetate. Goserelin can be administered under the trade name Zoladex™

In some embodiments, topoisomerase I inhibitors include, but are not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

In some embodiments, topoisomerase II inhibitors include but are not limited to the anthracyclines such as doxorubicin (including liposomal formulation, such as Caelyx™), daunorubicin, epirubicin, idarubicin and nemorubicin, the anthraquinones mitoxantrone and losoxantrone, and the podophillotoxines etoposide and teniposide. Etoposide is marketed under the trade name Etopophos™ Teniposide is marketed under the trade name VM 26-Bristol Doxorubicin is marketed under the trade name Acriblastin™ or Adriamycin™. Epirubicin is marketed under the trade name Farmorubicin™ Idarubicin is marketed. under the trade name Zavedos™. Mitoxantrone is marketed under the trade name Novantron.

In some embodiments, a microtubule active agent is microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™. Docetaxel is marketed under the trade name Taxotere™. Vinblastine sulfate is marketed under the trade name Vinblastin R.P™. Vincristine sulfate is marketed under the trade name Farmistin™

In some embodiments, alkylating agents include, but are not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

In some embodiments, histone deacetylase inhibitors or HDAC inhibitors are compounds which inhibit the histone deacetylase and which possess antiproliferative activity. This includes, but is not limited to, suberoylanilide hydroxamic acid (SAHA).

In some embodiments, antineoplastic antimetabolites include, but are not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

In some embodiments, platin compounds include, but are not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

In some embodiments, compounds targeting/decreasing a protein or lipid kinase activity; or a protein or lipid phosphatase activity; or further anti-angiogenic compounds include but are not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; lsis 3521; LY333531/LY379196; isochinoline compounds; FTIs; PD184352 or QAN697 (a P13K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, Cl-1033, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

In some embodiments, PI3K inhibitors include, but are not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

In some embodiments, BTK inhibitors include, but are not limited to compounds having inhibitory activity against Bruton's Tyrosine Kinase (BTK), including, but not limited to AVL-292 and ibrutinib.

In some embodiments, SYK inhibitors include, but are not limited to compounds having inhibitory activity against spleen tyrosine kinase (SYK), including but not limited to PRT-062070, R-343, R-333, Excellair, PRT-062607, and fostamatinib

Further examples of BTK inhibitory compounds, and conditions treatable by such compounds in combination with compounds of this disclosure can be found in WO2008039218 and WO2011090760, the entirety of which are incorporated herein by reference.

Further examples of SYK inhibitory compounds, and conditions treatable by such compounds in combination with compounds of this disclosure can be found in WO2003063794, WO2005007623, and WO2006078846, the entirety of which are incorporated herein by reference.

Further examples of PI3K inhibitory compounds, and conditions treatable by such compounds in combination with compounds of this disclosure can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this disclosure can be found in WO2009114512, WO2008109943, WO2007053452, WO2000142246, and WO2007070514, the entirety of which are incorporated herein by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the present disclosure include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

In some embodiments, cyclooxygenase inhibitors include, but are not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

In some embodiments, bisphosphonates include, but are not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™ Zoledronic acid is marketed under the trade name Zometa™. In some embodiments, mTOR inhibitors are compounds which inhibit the mammalian target of rapamycin (mTOR) and which possess antiproliferative activity such as sirolimus (Rapamune®), everolimus (Certican™), CCI-779 and ABT578.

In some embodiments, heparanase inhibitors are compounds which target, decrease or inhibit heparin sulfate degradation. They include, but are not limited to, PI-88. In some embodiments, a biological response modifier is a lymphokine or interferons.

In some embodiments, inhibitor of Ras oncogenic isoforms, such as H-Ras, K-Ras, or N-Ras, are compounds which target, decrease or inhibit the oncogenic activity of Ras; for example, a “farnesyl transferase inhibitor” such as L-744832, DK8G557 or R115777 (Zarnestra™). In some embodiments, telomerase inhibitors are compounds which target, decrease or inhibit the activity of telomerase. Compounds which target, decrease or inhibit the activity of telomerase are especially compounds which inhibit the telomerase receptor, such as telomestatin.

In some embodiments, methionine aminopeptidase inhibitors are compounds which target, decrease or inhibit the activity of methionine aminopeptidase. Compounds which target, decrease or inhibit the activity of methionine aminopeptidase include, but are not limited to, bengamide or a derivative thereof.

In some embodiments, proteasome inhibitors are compounds which target, decrease or inhibit the activity of the proteasome. Compounds which target, decrease or inhibit the activity of the proteasome include, but are not limited to, Bortezomib (Velcade™) and MLN 341.

In some embodiments, matrix metalloproteinase inhibitors or (“MMP” inhibitors) include, but are not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

In some embodiments, compounds used in the treatment of hematologic malignancies include, but are not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

In some embodiments, HSP90 inhibitors include, but are not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

In some embodiments, antiproliferative antibodies include but are not limited to, trastuzumab (Herceptin™), Trastuzumab-DM1, erbitux, bevacizumab (Avastin™), rituximab (Rituxan®), PR064553 (anti-CD40) and 2C4 Antibody. In some embodiments, antibodies are intact monoclonal antibodies, polyclonal antibodies, multispecific antibodies formed from at least 2 intact antibodies, or antibody fragments so long as they exhibit a desired biological activity.

For the treatment of acute myeloid leukemia (AML), compounds of the present disclosure can be used in combination with standard leukemia therapies, especially in combination with therapies used for the treatment of AML. In particular, compounds of the present disclosure can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. In some embodiments, somatostatin receptor antagonists are compounds which target, treat or inhibit the somatostatin receptor such as octreotide, and SOM230. In some embodiments, tumor cell damaging approaches are approaches such as ionizing radiation. In some embodiments, ionizing radiation is ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. In some embodiments, EDG binders are a class of immunosuppressants that modulates lymphocyte recirculation, such as FTY720. In some embodiments, ribonucleotide reductase inhibitors are pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy is in some embodiments therapy which uses certain chemicals known as photosensitizing compounds to treat or prevent cancers. Examples of photodynamic therapy include treatment with compounds, such as Visudyne™ and porfimer sodium.

In some embodiments, angiostatic steroids are compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

In some embodiments, implants containing corticosteroids are compounds, such as fluocinolone and dexamethasone.

Other chemotherapeutic compounds include, but are not limited to, plant alkaloids, hormonal compounds and antagonists; biological response modifiers, e.g., lymphokines or interferons; antisense oligonucleotides or oligonucleotide derivatives; shRNA or siRNA; or miscellaneous compounds or compounds with other or unknown mechanism of action.

In some embodiments, compounds of the present disclosure are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosing or potential side effects of such drugs. In some embodiments, a compound of the present disclosure may be mixed with the other drug substance in a fixed pharmaceutical composition or it may be administered separately, before, simultaneously with or after the other drug substance. Accordingly the present disclosure provides a combination of a provided compound as hereinbefore described with an anti-inflammatory, bronchodilatory, antihistamine or anti-tussive drug substance, said provided compound and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vernalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

Structures of active compounds identified by code numbers, generic or trade names may be taken from the actual edition of the standard compendium “The Merck Index” or from databases, e.g. Patents International (e.g. IMS World Publications).

A compound of the present disclosure may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the present disclosure can be administered alone or in combination with one or more other therapeutic compounds, possible combination therapy taking the form of fixed combinations or the administration of a compound of the present disclosure and one or more other therapeutic compounds being staggered or given independently of one another, or the combined administration of fixed combinations and one or more other therapeutic compounds. A compound of the present disclosure can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

In some embodiments, compositions are formulated so that a dosage of between 0.01-100 mg/kg body weight/day of a compound can be administered.

In some embodiments, in compositions which comprise an additional therapeutic agent, that additional therapeutic agent and the compound of the present disclosure may act synergistically. In some embodiments, the amount of additional therapeutic agent is be less than that required in a monotherapy utilizing only that therapeutic agent. In such compositions a dosage of between 0.01-1,000 μg/kg body weight/day of the additional therapeutic agent can be administered.

Compounds of the present disclosure, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a compound of the present disclosure. Implantable devices coated with a compound of the present disclosure are another embodiment of the present disclosure.

Among other things, the present disclosure provides the following Embodiments:

1. An agent comprising:

an antibody binding moiety,

a target binding moiety, and

optionally a linker moiety,

wherein the target binding moiety binds specifically to CD38.
2. The agent of Embodiment 1, wherein the agent has the structure of formula I:

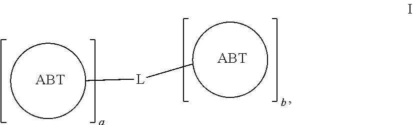

or a pharmaceutically acceptable salt thereof, wherein:

each of a and b is independently 1-200;

each ABT is independently an antibody binding moiety;

L is a linker moiety that connects ABT with TBT; and

each TBT is independently a target binding moiety.

3. The agent of Embodiment 1, wherein the agent has the structure of:

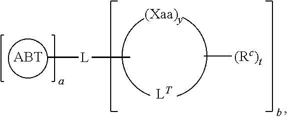

or a pharmaceutically acceptable salt thereof, wherein:

each of a and b is independently 1-200;

each ABT is independently an antibody binding moiety;

L is a bivalent linker moiety that connects ABT with TBT;

each Xaa is independently a residue of an amino acid or an amino acid analog;

y is 5-20;

L^{T} is a linker moiety linking two residues each independently of an amino acid or an amino acid analog, and is independently a covalent bond, or an optionally substituted bivalent group selected from C₁-C₆ aliphatic or C₁-C₆ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;

each R^{c} is independently -L^{a}-R′;

t is 0-50;

each L^{a} is independently a covalent bond, or an optionally substituted bivalent group selected from C₁-C₅₀ aliphatic or C₁-C₅₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;

each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;

each R′ is independently —R, —C(O)R, —CO₂R, or —SO₂R;

each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

4. The agent of any one of the preceding Embodiments, wherein a is 1.
5. The agent of any one of the preceding Embodiments, wherein b is 1.
6. The agent of any one of the preceding Embodiments, wherein the target binding moiety is or comprises (Xaa)y, wherein each Xaa is independently a residue of an amino acid or an amino acid analog, and y is 5-20.
7. An agent comprising:

an antibody binding moiety,

a target binding moiety, and

optionally a linker moiety,

wherein the target binding moiety has the structure of:

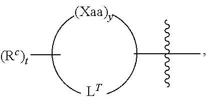

or a salt thereof, wherein:

each Xaa is independently a residue of an amino acid or an amino acid analog;

y is 5-20;

L^{T} is a linker moiety linking two residues each independently of an amino acid or an amino acid analog, and is independently a covalent bond, or an optionally substituted bivalent group selected from C₁-C₆ aliphatic or C₁-C₆ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;

each R^{c} is independently -L^{a}-R′;

t is 0-50;

each L^{a} is independently a covalent bond, or an optionally substituted bivalent group selected from C₁-C₅₀ aliphatic or C₁-C₅₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;

each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;

each R′ is independently —R, —C(O)R, —CO₂R, or —SO₂R;

each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or

two R groups are optionally and independently taken together to form a covalent bond, or:

two or more R groups on the same atom are optionally and independently taken together with the atom to form an optionally substituted, 3-30 membered, monocyclic, bicyclic or polycyclic ring having, in addition to the atom, 0-10 heteroatoms; or

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

8. The agent of any one of the preceding Embodiments, wherein the antibody binding moiety can bind to two or more antibodies which have different Fab regions.
9. The agent of any one of the preceding Embodiments, wherein the antibody binding moiety can bind to Fc regions.
10. The agent of any one of the preceding Embodiments, wherein the antibody binding moiety is or comprises optionally substituted

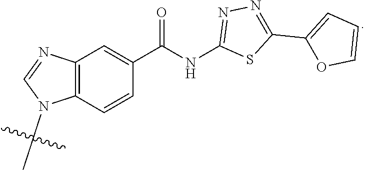 [SMILES: *n1cnc2cc(C(=O)Nc3nnc(-c4ccco4)s3)ccc21]

11. The agent of any one of the preceding Embodiments, wherein the antibody binding moiety is or comprises

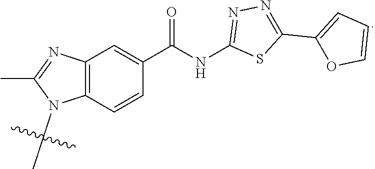 [SMILES: *n1c(C)nc2cc(C(=O)Nc3nnc(-c4ccco4)s3)ccc21]

12. The agent of any one of the preceding Embodiments, wherein the antibody binding moiety is or comprises optionally substitute

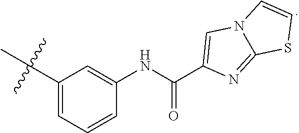 [SMILES: *c1cccc(NC(=O)c2cn3ccsc3n2)c1]

13. The agent of any one of the preceding Embodiments, wherein the antibody binding moiety is or comprises

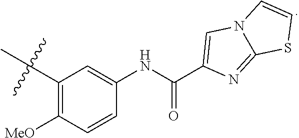 [SMILES: *c1cc(NC(=O)c2cn3ccsc3n2)ccc1OC]

14. The agent of any one of the preceding Embodiments, wherein the antibody binding moiety is or comprises optionally substituted

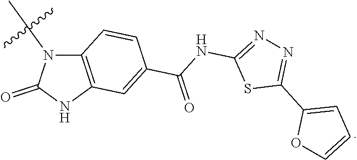 [SMILES: *n1c(=O)[nH]c2cc(C(=O)Nc3nnc(-c4ccco4)s3)ccc21]

15. The agent of any one of the preceding Embodiments, wherein the antibody binding moiety is or comprises

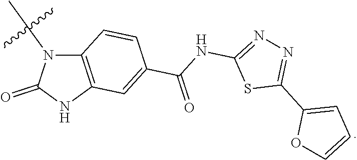 [SMILES: *n1c(=O)[nH]c2cc(C(=O)Nc3nnc(-c4ccco4)s3)ccc21]

16. The agent of any one of the preceding Embodiments, wherein the antibody binding moiety is or comprises optionally substituted

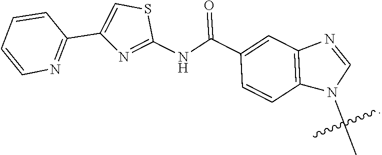 [SMILES: *n1cnc2cc(C(=O)Nc3nc(-c4ccccn4)cs3)ccc21]

17. The agent of any one of the preceding Embodiments, wherein the antibody binding moiety is or comprises

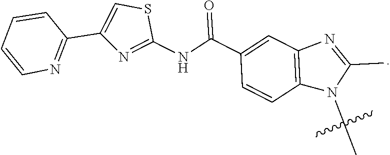 [SMILES: *n1c(C)nc2cc(C(=O)Nc3nc(-c4ccccn4)cs3)ccc21]

18. The agent of any one of the preceding Embodiments, wherein the antibody binding moiety is or comprises one or more amino acid residues.
19. The agent of any one of the preceding Embodiments, wherein the antibody binding moiety is or comprises a peptide moiety.
20. The agent of any one of the preceding Embodiments, wherein the antibody binding moiety is or comprises R^{c}-(Xaa)z- or

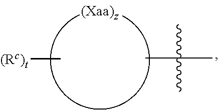

or a salt form thereof.
21. The agent of any one of the preceding Embodiments, wherein the antibody binding moiety is or comprises

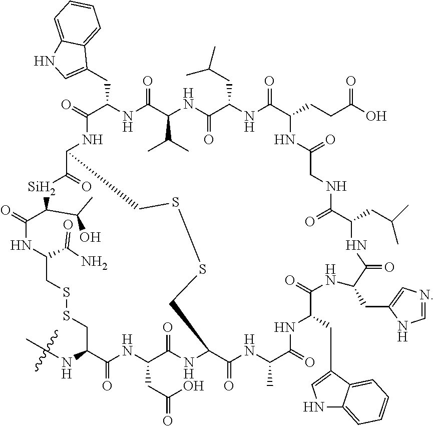

22. The agent of any one of the preceding Embodiments, wherein the antibody binding moiety is or comprises

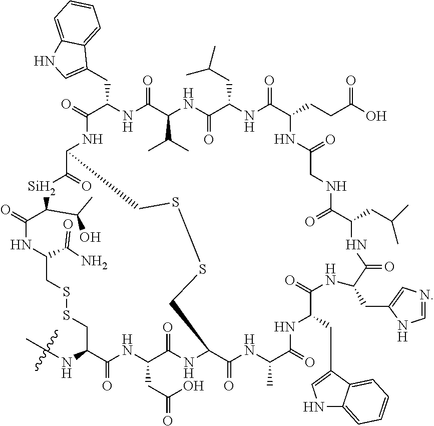

23. The agent of any one of the preceding Embodiments, wherein the antibody binding moiety is or comprises

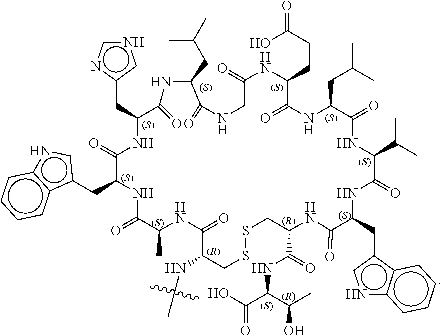

24. The agent of any one of the preceding Embodiments, wherein the antibody binding moiety is or comprises

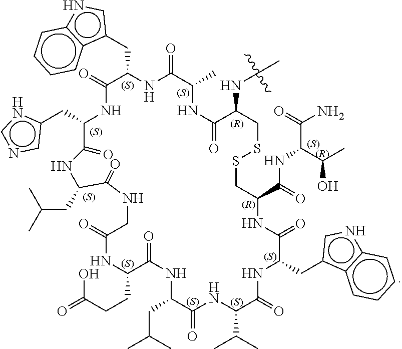

25. The agent of any one of Embodiments 1-9, wherein the antibody binding moiety has the structure of is R^{c}-(Xaa)z- or

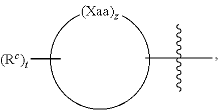

or a salt form thereof.
26. The agent of any one of Embodiments 1-9 and 25, wherein the antibody binding moiety has the structure of DCAWHLGELVWCT or a salt form thereof, wherein the two C residues are linked by a —S—S—.
27. The agent of any one of the preceding Embodiments, wherein -(Xaa)y- comprises:

-Xaa^{T1}-Xaa^{T2}-(Xaa)y′-Xaa^{T3}-Xaa^{T4}-Xaa^{T5}-,

wherein:

y′ is 0-8;

Xaa^{T1} is a residue of an amino acid or an amino acid analog whose side chain is substituted C₁-C₈ aliphatic;

Xaa^{T2} is a residue of an amino acid or an amino acid analog whose side chain comprises an optionally substituted aromatic group or is optionally substituted C₃-C₈ aliphatic;

Xaa^{T3} is a residue of an amino acid or an amino acid analog whose side chain is optionally substituted C₂-C₈ aliphatic;

Xaa^{T4} is a residue of an amino acid or an amino acid analog whose side chain comprises an optionally substituted aromatic group, or is optionally substituted C₃-C₈ aliphatic; and

Xaa^{T5} is a residue of an amino acid or an amino acid analog whose side chain is substituted C₁-C₈ aliphatic.

28. The agent of Embodiment 27, wherein y′ is 4.
29. The agent of any one of Embodiments 27-28, wherein Xaa^{T1} is a residue whose side chain is optionally substituted C₂-C₈ alkyl.
30. The agent of any one of Embodiments 27-28, wherein Xaa^{T1} is a residue whose side chain is unsubstituted linear C₂-C₈ alkyl.
31. The agent of any one of Embodiments 27-28, wherein Xaa^{T1} is a residue whose side chain is unsubstituted linear C₂-C₈ alkyl.
32. The agent of any one of Embodiments 27-28, wherein Xaa^{T1} is a residue whose side chain is n-pentyl.
33. The agent of any one of Embodiments 27-28, wherein Xaa^{T1} is a residue of Ahp, Y, W, S, K or K(MePEG4c).
34. The agent of any one of Embodiments 27-28, wherein Xaa^{T1} is a residue of Ahp.
35. The agent of any one of Embodiments 27-28, wherein Xaa^{T1} is a residue of Y.
36. The agent of any one of Embodiments 27-28, wherein Xaa^{T1} is a residue of W.
37. The agent of any one of Embodiments 27-28, wherein Xaa^{T1} is a residue of S.
38. The agent of any one of Embodiments 27-28, wherein Xaa^{T1} is a residue of K.
39. The agent of any one of Embodiments 27-28, wherein Xaa^{T1} is a residue of K(MePEG4c).
40. The agent of any one of Embodiments 27-38, wherein Xaa^{T2} is a residue whose side chain is —CH₂—R, wherein R is optionally substituted phenyl.
41. The agent of any one of Embodiments 27-38, wherein Xaa^{T2} is a residue whose side chain is optionally substituted C₃-C₈ aliphatic.
42. The agent of any one of Embodiments 27-38, wherein Xaa^{T2} is a residue of Y, W, Ahp, Bph, L or A.
43. The agent of any one of Embodiments 27-38, wherein Xaa^{T2} is a residue of Y.
44. The agent of any one of Embodiments 27-38, wherein Xaa^{T2} is a residue of W.
45. The agent of any one of Embodiments 27-38, wherein Xaa^{T2} is a residue of Ahp.
46. The agent of any one of Embodiments 27-38, wherein Xaa^{T2} is a residue of Bph.
47. The agent of any one of Embodiments 27-38, wherein Xaa^{T2} is a residue of L.
48. The agent of any one of Embodiments 27-38, wherein Xaa^{T2} is a residue of A.
49. The agent of any one of Embodiments 27-48, wherein Xaa^{T3} is a residue of L, Ahp, V, T, Hse, or MetO2.
50. The agent of any one of Embodiments 27-48, wherein Xaa^{T3} is a residue of L.
51. The agent of any one of Embodiments 27-48, wherein Xaa^{T3} is a residue of Ahp.
52. The agent of any one of Embodiments 27-48, wherein Xaa^{T3} is a residue of V.
53. The agent of any one of Embodiments 27-48, wherein Xaa^{T3} is a residue of T.
54. The agent of any one of Embodiments 27-48, wherein Xaa^{T3} is a residue of Hse.
55. The agent of any one of Embodiments 27-48, wherein Xaa^{T3} is a residue of MetO2.
56. The agent of any one of Embodiments 27-55, wherein Xaa^{T4} is a residue whose side chain comprises an optionally substituted aromatic group.
57. The agent of any one of Embodiments 27-55, wherein Xaa^{T4} is a residue whose side chain —CH₂—R, wherein R is optionally substituted phenyl.
58. The agent of any one of Embodiments 27-55, wherein Xaa^{T4} is a residue whose side chain comprises an optionally substituted C₃-C₈ aliphatic.
59. The agent of any one of Embodiments 27-55, wherein Xaa^{T4} is a residue of Bph, V or Ahp.
60. The agent of any one of Embodiments 27-55, wherein Xaa^{T4} is a residue of Bph.
61. The agent of any one of Embodiments 27-60, wherein Xaa^{T5} is a residue whose side chain is optionally substituted C₂-C₆ alkyl.
62. The agent of any one of Embodiments 27-60, wherein Xaa^{T5} is a residue whose side chain is optionally substituted C₂-C₆ linear alkyl.
63. The agent of any one of Embodiments 27-60, wherein Xaa^{T5} is a residue whose side chain is n-pentyl.
64. The agent of any one of Embodiments 27-60, wherein Xaa^{T5} is a residue of Ahp, Bph, Ado, Ano, PhNle or PhNva.
65. The agent of any one of Embodiments 27-60, wherein Xaa^{T5} is a residue of Ahp.
66. The agent of any one of Embodiments 27-60, wherein Xaa^{T5} is a residue of Bph.
67. The agent of any one of Embodiments 27-60, wherein Xaa^{T5} is a residue of Ado.
68. The agent of any one of Embodiments 27-60, wherein Xaa^{T5} is a residue of Ano.
69. The agent of any one of Embodiments 27-60, wherein Xaa^{T5} is a residue of PhNle
70. The agent of any one of Embodiments 27-60, wherein Xaa^{T5} is a residue of PhNva.
71. The agent of any one of Embodiments 27-70, wherein -(Xaa)y- is or comprises:

-(Xaa)ₐ₁-(Xaa)ₐ₂-(Xaa)ₐ₃-(Xaa)ₐ₄-(Xaa)ₐ₅-(Xaa)ₐ₆-(Xaa)ₐ₇-(Xaa)ₐ₈-(Xaa)ₐ₉-(Xaa)ₐ₁₀-(Xaa)ₐ₁₁-(Xaa)ₐ₁₂-(Xaa)ₐ₁₃-,

wherein:

each of a1, a2, a3, a4, a5, a6, a7, a8, a9, a10, a11, a12 and a13 is independently 0-5;

(Xaa)ₐ₃ is or comprises Xaa^{T1};

(Xaa)ₐ₄ is or comprises Xaa^{T2};

(Xaa)ₐ₉ is or comprises Xaa^{T3};

(Xaa)ₐ₁₀ is or comprises Xaa^{T4}; and

(Xaa)ₐ₁₁ is or comprises Xaa^{T5}

72. The agent of any one of Embodiments 27-71, wherein (Xaa)ₐ₁ is or comprises A, K or K(MePEG4c).
73. The agent of any one of Embodiments 27-71, wherein in (Xaa)ₐ₁, a1 is 1 and Xaa is a residue of A.
74. The agent of any one of Embodiments 27-71, wherein in (Xaa)ₐ₁, a1 is 1 and Xaa is a residue of K.
75. The agent of any one of Embodiments 27-71, wherein in (Xaa)ₐ₁, a1 is 1 and Xaa is a residue of K(MePEG4c).
76. The agent of any one of Embodiments 27-75, wherein (Xaa)ₐ₂ is or comprises R, S, D, Y, A, W, K, 4Py2NH2, Cit, F3G, hCit, K(MePEG4c), RNdMe, RNMe, or RNNdMe.
77. The agent of any one of Embodiments 27-75, wherein in (Xaa)ₐ₂, a2 is 1 and Xaa is a residue of R, S, D, Y, W, A or S.
78. The agent of any one of Embodiments 27-75, wherein in (Xaa)ₐ₂, a2 is 1 and Xaa is a residue of R, S, D, Y, W, A or S.
79. The agent of any one of Embodiments 27-75, wherein (Xaa)ₐ₂ is or comprises R.
80. The agent of any one of Embodiments 27-75, wherein (Xaa)ₐ₂ is or comprises S.
81. The agent of any one of Embodiments 27-75, wherein (Xaa)ₐ₂ is or comprises D.
82. The agent of any one of Embodiments 27-75, wherein (Xaa)ₐ₂ is or comprises Y.
83. The agent of any one of Embodiments 27-75, wherein (Xaa)ₐ₂ is or comprises A.
84. The agent of any one of Embodiments 27-75, wherein (Xaa)ₐ₂ is or comprises W.
85. The agent of any one of Embodiments 27-75, wherein (Xaa)ₐ₂ is or comprises K.
86. The agent of any one of Embodiments 27-75, wherein (Xaa)ₐ₂ is or comprises 4Py2NH2.
87. The agent of any one of Embodiments 27-75, wherein (Xaa)ₐ₂ is or comprises Cit.
88. The agent of any one of Embodiments 27-75, wherein (Xaa)ₐ₂ is or comprises F3G.
89. The agent of any one of Embodiments 27-75, wherein (Xaa)ₐ₂ is or comprises hCit.
90. The agent of any one of Embodiments 27-75, wherein (Xaa)ₐ₂ is or comprises K(MePEG4c).
91. The agent of any one of Embodiments 27-75, wherein (Xaa)ₐ₂ is or comprises RNdMe.
92. The agent of any one of Embodiments 27-75, wherein (Xaa)ₐ₂ is or comprises RNMe.
93. The agent of any one of Embodiments 27-75, wherein in (Xaa)ₐ₂, a2 is 1 and Xaa is a residue of RNNdMe.
94. The agent of any one of Embodiments 27-93, wherein (Xaa)ₐ₅ is or comprises H, Y, S, L, A, or W6N.
95. The agent of any one of Embodiments 27-93, wherein in (Xaa)ₐ₅, a5 is 1 and Xaa is a residue of H, Y, S, L, A, or W.
96. The agent of any one of Embodiments 27-93, wherein in (Xaa)ₐ₅, a5 is 1 and Xaa is a residue of H or Y.
97. The agent of any one of Embodiments 27-93, wherein in (Xaa)ₐ₅, a5 is 1 and Xaa is a residue of H.
98. The agent of any one of Embodiments 27-93, wherein in (Xaa)ₐ₅, a5 is 1 and Xaa is a residue of Y.
99. The agent of any one of Embodiments 27-93, wherein in (Xaa)ₐ₅, a5 is 1 and Xaa is a residue of S.
100. The agent of any one of Embodiments 27-93, wherein in (Xaa)ₐ₅, a5 is 1 and Xaa is a residue of L.
101. The agent of any one of Embodiments 27-93, wherein in (Xaa)ₐ₅, a5 is 1 and Xaa is a residue of A.
102. The agent of any one of Embodiments 27-93, wherein in (Xaa)ₐ₅, a5 is 1 and Xaa is a residue of W6N.
103. The agent of any one of Embodiments 27-102, wherein (Xaa)ₐ₆ is or comprises D, G, R, Y, H, W, A, or Y.
104. The agent of any one of Embodiments 27-102, wherein in (Xaa)ₐ₆, a6 is 1 and Xaa is a residue of D, G, R, Y, H, W, A, or Y.
105. The agent of any one of Embodiments 27-102, wherein in (Xaa)ₐ₆, a6 is 1 and Xaa is a residue of D, G, or R.
106. The agent of any one of Embodiments 27-102, wherein in (Xaa)ₐ₆, a6 is 1 and Xaa is a residue of D.
107. The agent of any one of Embodiments 27-102, wherein in (Xaa)ₐ₆, a6 is 1 and Xaa is a residue of A.
108. The agent of any one of Embodiments 27-107, wherein (Xaa)ₐ₇ is or comprises G, D, E, Q, N, R, MetO2, S, Har or A.
109. The agent of any one of Embodiments 27-107, wherein in (Xaa)ₐ₇ is or comprises G, D, E, Q, or N.
110. The agent of any one of Embodiments 27-107, wherein in (Xaa)ₐ₇, a7 is 1 and Xaa is a residue of G.
111. The agent of any one of Embodiments 27-107, wherein in (Xaa)ₐ₇, a7 is 1 and Xaa is a residue of D, G, or A.
112. The agent of any one of Embodiments 27-107, wherein in (Xaa)ₐ₇, a7 is 1 and Xaa is a residue of D.
113. The agent of any one of Embodiments 27-107, wherein in (Xaa)ₐ₇, a7 is 1 and Xaa is a residue of E.
114. The agent of any one of Embodiments 27-107, wherein in (Xaa)ₐ₇, a7 is 1 and Xaa is a residue of N.
115. The agent of any one of Embodiments 27-107, wherein in (Xaa)ₐ₇, a7 is 1 and Xaa is a residue of Q.
116. The agent of any one of Embodiments 27-107, wherein in (Xaa)ₐ₇, a7 is 1 and Xaa is a residue of MetO2.
117. The agent of any one of Embodiments 27-107, wherein in (Xaa)ₐ₇, a7 is 1 and Xaa is a residue of A.
118. The agent of any one of Embodiments 27-117, wherein (Xaa)ₐ₈ is or comprises V, A, D, G, W, S or T.
119. The agent of any one of Embodiments 27-117, wherein in (Xaa)ₐs, a8 is 1 and Xaa is a residue of V, A, D, G, W, S or T.
120. The agent of any one of Embodiments 27-117, wherein in (Xaa)ₐ₈, a8 is 1 and Xaa is a residue of V.
121. The agent of any one of Embodiments 27-117, wherein in (Xaa)ₐ₈, a8 is 1 and Xaa is a residue of A.
122. The agent of any one of Embodiments 27-121, wherein (Xaa)ₐ₁₂ is or comprises of D, A, S, G, or Ahp.
123. The agent of any one of Embodiments 27-121, wherein in (Xaa)ₐ₁₂, a12 is 1 and Xaa is a residue of D, S, G, or Ahp.
124. The agent of any one of Embodiments 27-121, wherein in (Xaa)ₐ₁₂, a12 is 1 and Xaa is a residue of D.
125. The agent of any one of Embodiments 27-121, wherein in (Xaa)ₐ₁₂, a12 is 1 and Xaa is a residue of A.
126. The agent of any one of Embodiments 27-125, wherein (Xaa)ₐ₁₃ is or comprises C.
127. The agent of any one of Embodiments 27-125, wherein in (Xaa)ₐ₁₃, a13 is 1 and Xaa is a residue of C.
128. The agent of any one of Embodiments 1-26, wherein -(Xaa)y- comprises:

-Xaa^{T6}-(Xaa)y′-Xaa^{T8}-Xaa^{T9}-Xaa^{T10}-Xaa^{T11}-,

wherein:

y′ is 0-8;

Xaa^{T6} is a residue of an amino acid or an amino acid analog whose side chain is substituted C₁-C₈ aliphatic;

Xaa^{T7} is a residue of an amino acid or an amino acid analog whose side chain is optionally substituted C₂-C₈ aliphatic;

Xaa^{T8} is a residue of proline or an amino acid analog thereof;

Xaa^{T9} is a residue of an amino acid or an amino acid analog whose side chain comprises an optionally substituted aromatic group, or is optionally substituted C₁-C₈ aliphatic;

Xaa^{T10} is a residue of an amino acid or an amino acid analog whose side chain is substituted C₁-C₈ aliphatic, or an amino acid whose amino group is substituted; and

Xaa^{T11} is a residue of an amino acid or an amino acid analog whose side chain comprises an optionally substituted aromatic group, or is optionally substituted C₁-C₈ aliphatic.

129. The agent of Embodiment 128, wherein y′ is 1.
130. The agent of any one of Embodiments 128-129, wherein the side chain of Xaa^{T6} is —CH₂—R, wherein R is optionally substituted phenyl.
131. The agent of any one of Embodiments 128-130, wherein Xaa^{T6} is an amino acid residue whose amino group has the structure of —N(R)—, wherein R is optionally substituted C₁₋₆ aliphatic.
132. The agent of any one of Embodiments 128-131, wherein Xaa^{T6} is an amino acid residue whose amino group has the structure of —N(Me)-.
133. The agent of any one of Embodiments 128-129, wherein Xaa^{T6} is a residue of MeF, L, or S.
134. The agent of any one of Embodiments 128-129, wherein Xaa^{T6} is a residue of MeF.
135. The agent of any one of Embodiments 128-134, wherein Xaa^{T7} is a residue of L or MeF.
136. The agent of any one of Embodiments 128-134, wherein Xaa^{T7} is a residue of L.
137. The agent of any one of Embodiments 128-136, wherein Xaa^{T8} is a residue of P.
138. The agent of any one of Embodiments 128-137, wherein Xaa^{T9} is a residue whose side chain comprises an optionally substituted aromatic group.
139. The agent of any one of Embodiments 128-138, wherein Xaa^{T9} is a residue whose side chain is —CH₂—R, wherein R is optionally substituted phenyl.
140. The agent of any one of Embodiments 128-137, wherein Xaa^{T9} is a residue whose side chain is optionally substituted C₁-C₈ aliphatic.
141. The agent of any one of Embodiments 128-137, wherein Xaa^{T9} is a residue of Bph, D or S.
142. The agent of any one of Embodiments 128-137, wherein Xaa^{T9} is a residue of Bph.
143. The agent of any one of Embodiments 128-142, wherein Xaa^{T10} is a residue whose side chain is substituted C₁-C₈ aliphatic.
144. The agent of any one of Embodiments 128-142, wherein Xaa^{T10} is a residue of V or L.
145. The agent of any one of Embodiments 128-142, wherein Xaa^{T10} is a residue of V.
146. The agent of any one of Embodiments 128-142, wherein Xaa^{T10} is a residue comprising a substituted amino group.
147. The agent of any one of Embodiments 128-142, wherein Xaa^{T10} is a residue of MeG.
148. The agent of any one of Embodiments 128-147, wherein Xaa^{T11} is a residue whose side chain comprises an optionally substituted aromatic group.
149. The agent of any one of Embodiments 128-147, wherein Xaa^{T11} is a residue whose side chain is —CH₂—R, wherein R is optionally substituted aryl or heteroaryl.
150. The agent of any one of Embodiments 128-147, wherein Xaa^{T11} is a residue of W.
151. The agent of any one of Embodiments 128-147, wherein Xaa^{T11} is a residue whose side chain is optionally substituted C₁-C₈ aliphatic.
152. The agent of any one of Embodiments 128-147, wherein Xaa^{T11} is a residue of R. 153. The agent of any one of Embodiments 128-152, wherein -(Xaa)y- is or comprises:

-(Xaa)ₐ₁-(Xaa)ₐ₂-(Xaa)ₐ₃-(Xaa)ₐ₄-(Xaa)ₐ₅-(Xaa)ₐ₆-(Xaa)ₐ₇-(Xaa)ₐ₈-(Xaa)ₐ₉-(Xaa)ₐ₁₀-(Xaa)ₐ₁₁-(Xaa)ₐ₁₂-,

wherein:

each of a1, a2, a3, a4, a5, a6, a7, a8, a9, a10, a11, and a12 is independently 0-5;

(Xaa)ₐ₄ is or comprises Xaa^{T6};

(Xaa)ₐ₆ is or comprises Xaa^{T7};

(Xaa)ₐ₇ is or comprises Xaa^{T8};

(Xaa)ₐ₈ is or comprises Xaa^{T9};

(Xaa)ₐ₉ is or comprises Xaa^{T10}; and

(Xaa)ₐ₁₀ is or comprises Xaa^{T11}.

154. The agent of any one of Embodiments 128-153, wherein (Xaa)ₐ₁ is or comprises A.
155. The agent of any one of Embodiments 128-153, wherein in (Xaa)ₐ₁, a1 is 1 and Xaa is a residue of A.
156. The agent of any one of Embodiments 128-155, wherein (Xaa)ₐ₂ is or comprises L, A or P.
157. The agent of any one of Embodiments 128-155, wherein in (Xaa)ₐ₂, a2 is 1 and Xaa is a residue of L.
158. The agent of any one of Embodiments 128-155, wherein in (Xaa)ₐ₂, a2 is 1 and Xaa is a residue of A.
159. The agent of any one of Embodiments 128-158, wherein (Xaa)₃ is or comprises H, R, or A.
160. The agent of any one of Embodiments 128-158, wherein in (Xaa)ₐ₃, a3 is 1 and Xaa is a residue of H, R, or A.
161. The agent of any one of Embodiments 128-158, wherein in (Xaa)ₐ₃, a3 is 1 and Xaa is a residue of H, R, or A.
162. The agent of any one of Embodiments 128-158, wherein in (Xaa)ₐ₃, a3 is 1 and Xaa is a residue of H.
163. The agent of any one of Embodiments 128-158, wherein in (Xaa)ₐ₃, a3 is 1 and Xaa is a residue of A.
164. The agent of any one of Embodiments 128-163, wherein (Xaa)ₐ₅ is or comprises V, A or MeG.
165. The agent of any one of Embodiments 128-163, wherein in (Xaa)ₐ₅, a5 is 1 and Xaa is a residue of V, A or MeG.
166. The agent of any one of Embodiments 128-163, wherein in (Xaa)ₐ₅, a5 is 1 and Xaa is a residue of V.
167. The agent of any one of Embodiments 128-163, wherein in (Xaa)ₐ₅, a5 is 1 and Xaa is a residue of A.
168. The agent of any one of Embodiments 128-167, wherein (Xaa)ₐ₁₁ is or comprises V, A, D or MeG.
169. The agent of any one of Embodiments 128-167, wherein in (Xaa)ₐ₁₁, a11 is 1 and Xaa is a residue of V, A, D or MeG.
170. The agent of any one of Embodiments 128-167, wherein in (Xaa)ₐ₁₁, a11 is 1 and Xaa is a residue of V.
171. The agent of any one of Embodiments 128-167, wherein in (Xaa)ₐ₁₁, a11 is 1 and Xaa is a residue of A.
172. The agent of any one of Embodiments 128-167, wherein (Xaa)ₐ₁₂ is or comprises C.
173. The agent of any one of Embodiments 128-167, wherein in (Xaa)ₐ₁₂, a12 is 1 and Xaa is a residue of C.
174. The agent of any one of Embodiments 27-173, wherein two Xaa are linked together.
175. The agent of any one of Embodiments 27-174, wherein two Xaa are linked together through a linker having the structure of —C(O)—CH₂—.
176. The agent of Embodiment 175, wherein —C(O)— is bonded to an amino group of a Xaa.
177. The agent of Embodiment 176, wherein the Xaa is a N-terminal residue.
178. The agent of any one of Embodiments 175-177, wherein —CH₂— is bonded to —S— in the side chain of a Xaa.
179. The agent of Embodiment 178, wherein the Xaa is a C-terminal residue.
180. The agent of Embodiment 178 or 179, wherein the Xaa is a C.
181. The agent of any one of Embodiments 1-127 and 174-180, wherein target binding moiety or

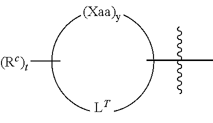

is or comprises

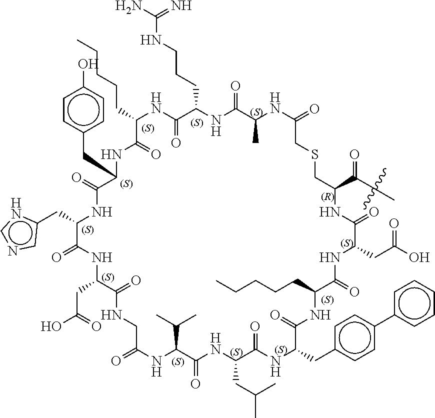

or a salt form thereof.
182. The agent of any one of Embodiments 1-127 and 174-180, wherein target binding moiety or

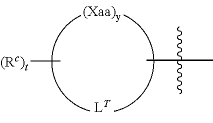

is or comprises

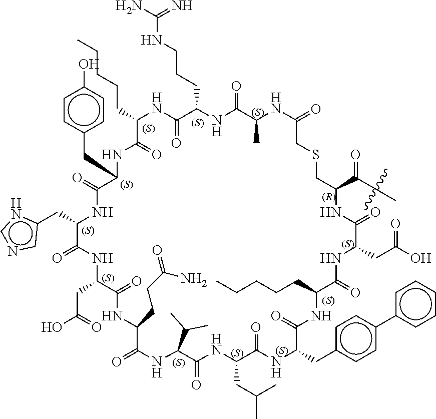

or a salt form thereof.
183. The agent of any one of Embodiments 1-127 and 174-180, wherein target binding moiety or

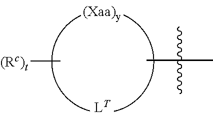

is or comprises

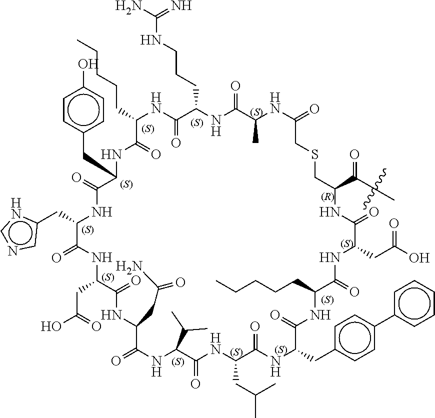

or a salt form thereof.
184. The agent of any one of Embodiments 1-127 and 174-180, wherein target binding moiety or

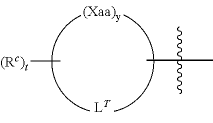

is or comprises

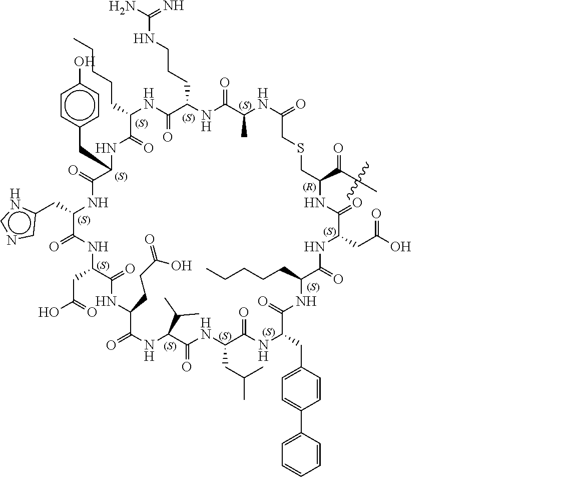

or a salt form thereof.
185. The agent of any one of Embodiments 1-127 and 174-180, wherein target binding moiety or

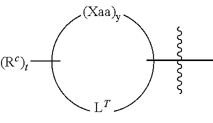

is or comprises

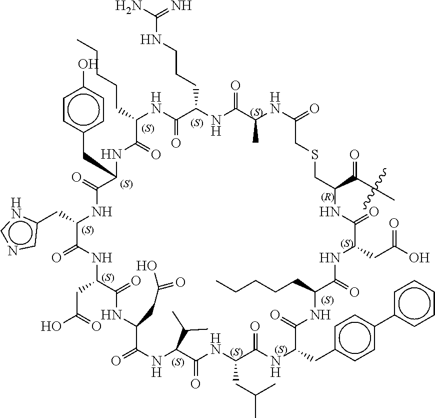

or a salt form thereof.
186. The agent of any one of Embodiments 1-26 and 128-180, wherein target binding moiety or

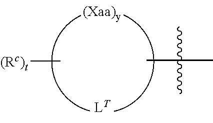

is or comprises

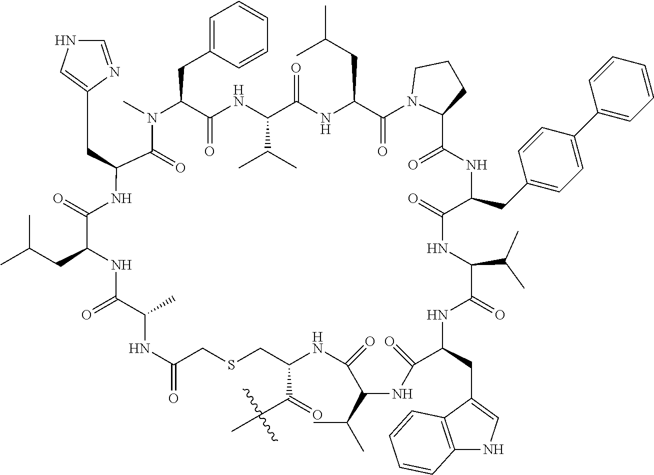

or a salt form thereof.
187. The agent of any one of Embodiments 1-26 and 128-180, wherein target binding moiety or

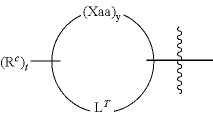

is or comprises

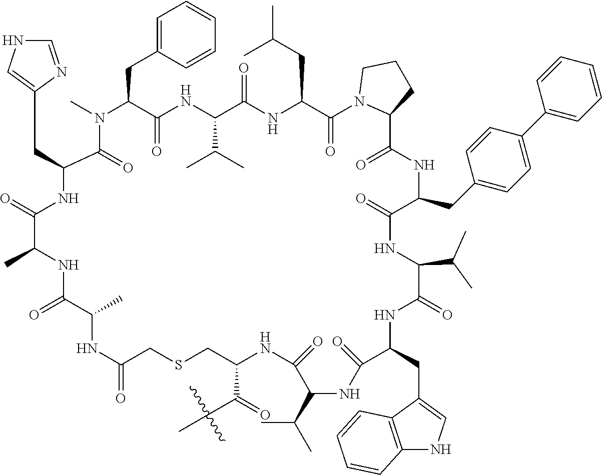

or a salt form thereof.
188. The agent of any one of Embodiments 1-26, wherein target binding moiety or -(Xaa)y- is or comprises a peptide that is:

(1) a polypeptide having an amino acid sequence represented by any one of SEQ ID NOS. 1-34:

(2) a polypeptide having an amino acid sequence represented by any one of SEQ ID NOS. 1-34 wherein the amino acid residue at the N-terminal is a chloroacetylated (e.g., at its amino group);

(3) a polypeptide having an amino acid sequence with deletions, additions, substitutions or insertion of one or more amino acids in any one of SEQ ID NOS. 1-34, which does not comprises an amino acid sequence with deletion of Cys at the C terminal in SEQ ID NOS. 1-34;

(4) a polypeptide having an amino acid sequence represented by any one of SEQ ID NOS. 1-34 with deletions, additions, substitutions or insertion of one or more amino acids in any one of SEQ ID NOS. 1-34, which does not comprises an amino acid sequence with deletion of Cys at the C terminal in any one of SEQ ID NOS. 1-34, wherein the amino acid at the N-terminal is a chloroacetylated (e.g., at its amino group); or

(5) a polypeptide in accordance with one of the above (1) to (4) wherein the polypeptide has a cyclized structure.

189. The agent of any one of Embodiments 1-26, wherein target binding moiety or -(Xaa)y- is or comprises a peptide that is:

(1) a polypeptide having an amino acid sequence represented by SEQ ID NO. 1 or 2:

Ala Arg Ahp Tyr His Asp Gly Val Leu Bph Ahp Asp Cys (SEQ ID NO.1),

Ala Leu His MePhe Val Leu Pro Bph Val Trp Val Cys (SEQ ID NO.2);

(2) a polypeptide having an amino acid sequence represented by SEQ ID NO. 1 or 2 wherein the Ala at the N-terminal is a chloroacetylated Ala;

(3) a polypeptide having an amino acid sequence with deletions, additions, substitutions or insertion of one or more amino acids in SEQ ID NO. 1 or 2, which does not comprises an amino acid sequence with deletion of Cys at the C terminal in SEQ ID NO. 1 or 2;

(4) a polypeptide having an amino acid sequence represented by SEQ ID NO. 1 or 2 wherein the Ala at the N-terminal is a chloroacetylated Ala with deletions, additions, substitutions or insertion of one or more amino acids in SEQ ID NO. 1 or 2, which does not comprises an amino acid sequence with deletion of Cys at the C terminal in SEQ ID NO. 1 or 2; or

(5) a polypeptide in accordance with one of the above (1) to (4) wherein the polypeptide has a cyclized structure.

190. The agent of any one of Embodiments 1-26, wherein target binding moiety or -(Xaa)y- is or comprises a peptide whose amino acid sequence is any one of SEQ ID NOs. 1-34.
191. The agent of any one of Embodiments 188-190, wherein target binding moiety or -(Xaa)y- has a cyclized structure.
192. The agent of any one of Embodiments 1-26, wherein a target binding moiety is derived from, or

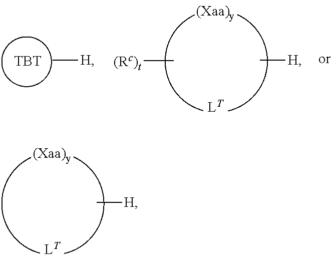

is a structure selected from S-1 to S-39 or a pharmaceutically acceptable salt thereof.
193. The agent of Embodiment 192, wherein the structure is S-1 or a pharmaceutically acceptable salt thereof.
194. The agent of Embodiment 192, wherein the structure is S-2 or a pharmaceutically acceptable salt thereof.
195. The agent of Embodiment 192, wherein the structure is S-3 or a pharmaceutically acceptable salt thereof.
196. The agent of Embodiment 192, wherein the structure is S-4 or a pharmaceutically acceptable salt thereof.
197. The agent of Embodiment 192, wherein the structure is S-5 or a pharmaceutically acceptable salt thereof.
198. The agent of Embodiment 192, wherein the structure is S-6 or a pharmaceutically acceptable salt thereof.
199. The agent of Embodiment 192, wherein the structure is S-7 or a pharmaceutically acceptable salt thereof.
200. The agent of Embodiment 192, wherein the structure is S-8 or a pharmaceutically acceptable salt thereof.
201. The agent of Embodiment 192, wherein the structure is S-9 or a pharmaceutically acceptable salt thereof.
202. The agent of Embodiment 192, wherein the structure is S-10 or a pharmaceutically acceptable salt thereof.
203. The agent of Embodiment 192, wherein the structure is 5-11 or a pharmaceutically acceptable salt thereof.
204. The agent of Embodiment 192, wherein the structure is S-12 or a pharmaceutically acceptable salt thereof.
205. The agent of Embodiment 192, wherein the structure is S-13 or a pharmaceutically acceptable salt thereof.
206. The agent of Embodiment 192, wherein the structure is S-14 or a pharmaceutically acceptable salt thereof.
207. The agent of Embodiment 192, wherein the structure is 5-15 or a pharmaceutically acceptable salt thereof.
208. The agent of Embodiment 192, wherein the structure is S-16 or a pharmaceutically acceptable salt thereof.
209. The agent of Embodiment 192, wherein the structure is S-17 or a pharmaceutically acceptable salt thereof.
210. The agent of Embodiment 192, wherein the structure is 5-18 or a pharmaceutically acceptable salt thereof.
211. The agent of Embodiment 192, wherein the structure is S-19 or a pharmaceutically acceptable salt thereof.
212. The agent of Embodiment 192, wherein the structure is S-20 or a pharmaceutically acceptable salt thereof.
213. The agent of Embodiment 192, wherein the structure is S-21 or a pharmaceutically acceptable salt thereof.
214. The agent of Embodiment 192, wherein the structure is S-22 or a pharmaceutically acceptable salt thereof.
215. The agent of Embodiment 192, wherein the structure is S-23 or a pharmaceutically acceptable salt thereof.
216. The agent of Embodiment 192, wherein the structure is S-24 or a pharmaceutically acceptable salt thereof.
217. The agent of Embodiment 192, wherein the structure is S-25 or a pharmaceutically acceptable salt thereof.
218. The agent of Embodiment 192, wherein the structure is S-26 or a pharmaceutically acceptable salt thereof.
219. The agent of Embodiment 192, wherein the structure is S-27 or a pharmaceutically acceptable salt thereof.
220. The agent of Embodiment 192, wherein the structure is S-28 or a pharmaceutically acceptable salt thereof.
221. The agent of Embodiment 192, wherein the structure is S-29 or a pharmaceutically acceptable salt thereof.
222. The agent of Embodiment 192, wherein the structure is S-30 or a pharmaceutically acceptable salt thereof.
223. The agent of Embodiment 192, wherein the structure is S-31 or a pharmaceutically acceptable salt thereof.
224. The agent of Embodiment 192, wherein the structure is S-32 or a pharmaceutically acceptable salt thereof.
225. The agent of Embodiment 192, wherein the structure is S-33 or a pharmaceutically acceptable salt thereof.
226. The agent of Embodiment 192, wherein the structure is S-34 or a pharmaceutically acceptable salt thereof.
227. The agent of Embodiment 192, wherein the structure is S-35 or a pharmaceutically acceptable salt thereof.
228. The agent of Embodiment 192, wherein the structure is S-36 or a pharmaceutically acceptable salt thereof.
229. The agent of Embodiment 192, wherein the structure is S-37 or a pharmaceutically acceptable salt thereof.
230. The agent of Embodiment 192, wherein the structure is S-38 or a pharmaceutically acceptable salt thereof.
231. The agent of Embodiment 192, wherein the structure is S-39 or a pharmaceutically acceptable salt thereof.
232. The agent of any one of the preceding Embodiments, comprising a linker.
233. The agent of any one of the preceding Embodiments, wherein the linker is or comprises —(CH₂CH₂O)n-, wherein n is 1-20.
234. The agent of any one of the preceding Embodiments, wherein the linker is or comprises —(CH₂CH₂O)n-, wherein n is about 5-20.
235. The agent of any one of the preceding Embodiments, wherein the linker is or comprises —(CH₂CH₂O)n-, wherein n is about 10-20.
236. The agent of any one of the preceding Embodiments, wherein the linker comprises one or more amino acid residues.
237. The agent of any one of the preceding Embodiments, wherein the linker comprises one or more natural amino acid residues.
238. The agent of any one of the preceding Embodiments, wherein the linker comprises one or more unnatural amino acid residues.
239. The agent of any one of the preceding Embodiments, wherein the linker comprises one or more D-amino acid residues.
240. The agent of any one of the preceding Embodiments, wherein the linker is or comprises a glycine residue.
241. The agent of any one of the preceding Embodiments, wherein the linker is or comprises a beta-alanine residue.
242. The agent of any one of the preceding Embodiments, wherein the linker is or comprises a residue having the structure of —C(O)—(CH₂CH₂O)n-CH₂CH₂NR′— or a salt form thereof, wherein n is 0-20.
243. The agent of any one of the preceding Embodiments, wherein the linker is or comprises a residue having the structure of —C(O)—(CH₂CH₂O)n-CH₂CH₂NR′— or a salt form thereof, wherein n is 0-12.
244. The agent of any one of Embodiments 242-243, wherein R′ is —H.
245. The agent of any one of the preceding Embodiments, wherein the linker is or comprises -Gly-Gly-.
246. The agent of any one of the preceding Embodiments, wherein the linker is or comprises
247. The agent of any one of the preceding Embodiments, wherein the linker comprises a cycloaddition product moiety.
248. The agent of any one of the preceding Embodiments, wherein the linker comprises

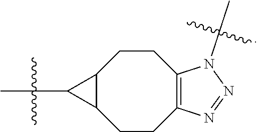 [SMILES: *C1C2CCc3nnn(*)c3CCC12]

249. An agent, wherein the agent is I-3 or a pharmaceutically acceptable salt thereof.
250. An agent, wherein the agent is I-5 or a pharmaceutically acceptable salt thereof.
251. An agent, wherein the agent is I-6 or a pharmaceutically acceptable salt thereof.
252. An agent, wherein the agent is I-7 or a pharmaceutically acceptable salt thereof.
253. An agent, wherein the agent is I-8 or a pharmaceutically acceptable salt thereof.
254. An agent, wherein the agent is I-9 or a pharmaceutically acceptable salt thereof.
255. An agent, wherein the agent is I-10 or a pharmaceutically acceptable salt thereof.
256. An agent, wherein the agent is I-11 or a pharmaceutically acceptable salt thereof.
257. An agent, wherein the agent is I-12 or a pharmaceutically acceptable salt thereof.
258. An agent, wherein the agent is I-13 or a pharmaceutically acceptable salt thereof.
259. An agent, wherein the agent is I-15 or a pharmaceutically acceptable salt thereof.
260. An agent, wherein the agent is I-16 or a pharmaceutically acceptable salt thereof.
261. An agent, wherein the agent is I-17 or a pharmaceutically acceptable salt thereof.
262. An agent, wherein the agent is I-19 or a pharmaceutically acceptable salt thereof.
263. An agent, wherein the agent is I-24 or a pharmaceutically acceptable salt thereof.
264. The agent of any one of Embodiments 248-263, wherein

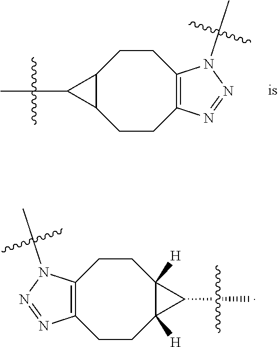

265. The agent of any one of Embodiments 248-264, wherein

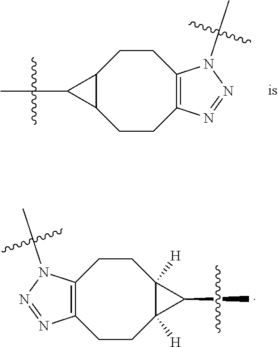

266. An agent, wherein the agent is I-1 or a pharmaceutically acceptable salt thereof.
267. An agent, wherein the agent is I-2 or a pharmaceutically acceptable salt thereof.
268. An agent, wherein the agent is I-4 or a pharmaceutically acceptable salt thereof.
269. An agent, wherein the agent is I-14 or a pharmaceutically acceptable salt thereof.
270. An agent, wherein the agent is I-18 or a pharmaceutically acceptable salt thereof.
271. An agent, wherein the agent is I-25 or a pharmaceutically acceptable salt thereof.
272. An agent, wherein the agent is I-26 or a pharmaceutically acceptable salt thereof.
273. An agent, wherein the agent is I-27 or a pharmaceutically acceptable salt thereof.
274. An agent, wherein the agent is I-28 or a pharmaceutically acceptable salt thereof.
275. An agent, wherein the agent is I-29 or a pharmaceutically acceptable salt thereof.
276. An agent, wherein the agent is I-30 or a pharmaceutically acceptable salt thereof.
277. An agent, wherein the agent is I-31 or a pharmaceutically acceptable salt thereof.
278. An agent, wherein the agent is I-32 or a pharmaceutically acceptable salt thereof.
279. An agent, wherein the agent is I-33 or a pharmaceutically acceptable salt thereof.
280. An agent, wherein the agent is I-34 or a pharmaceutically acceptable salt thereof.
281. An agent, wherein the agent is I-35 or a pharmaceutically acceptable salt thereof.
282. An agent, wherein the agent is I-36 or a pharmaceutically acceptable salt thereof.
283. An agent, wherein the agent is I-37 or a pharmaceutically acceptable salt thereof.
284. An agent, wherein the agent is I-38 or a pharmaceutically acceptable salt thereof.
285. An agent, wherein the agent is I-39 or a pharmaceutically acceptable salt thereof.
286. An agent, wherein the agent is I-40 or a pharmaceutically acceptable salt thereof.
287. An agent, wherein the agent is I-41 or a pharmaceutically acceptable salt thereof.
288. An agent, wherein the agent is I-42 or a pharmaceutically acceptable salt thereof.
289. An agent, wherein the agent is I-43 or a pharmaceutically acceptable salt thereof.
290. An agent, wherein the agent is I-44 or a pharmaceutically acceptable salt thereof.
291. An agent, wherein the agent is I-45 or a pharmaceutically acceptable salt thereof.
292. An agent, wherein the agent is I-46 or a pharmaceutically acceptable salt thereof.
293. An agent, wherein the agent is I-47 or a pharmaceutically acceptable salt thereof.
294. The agent of any one of the preceding Embodiments, wherein the agent specifically binds to CD38 as measured by SPR.
295. The agent of any one of the preceding Embodiments, wherein the agent binds to CD38 as measured by SPR with a Kd no more than 200, 100, 50, 40, 30, 20, 10 or 5 nM.
296. The agent of any one of the preceding Embodiments, wherein the agent binds to CD38 as measured under a condition described in the specification.
297. The agent of any one of the preceding Embodiments, wherein the agent binds to CD38 as measured under a condition described an Example.
298. The agent of any one of the preceding Embodiments, wherein the agent recruits antibodies to CD38-expressing target cells.
299. The agent of any one of the preceding Embodiments, wherein the agent provides less reduction of CD38-expressing non-diseased effector cells compared to a CD38 antibody.
300. A composition comprising an agent of any one of the preceding Embodiments, and a population of cells.
301. The composition of Embodiment 300, wherein the cells are manipulated cells.
302. The composition of any one of Embodiments 300-301, wherein the cells are manufactured cells.
303. The composition of any one of Embodiments 300-302, wherein the cells are useful for a cell-based therapy.
304. The composition of any one of Embodiments 300-303, wherein the cells are or comprise NK cells.
305. The composition of any one of Embodiments 300-304, wherein the cells are or comprise engineered NK cells.
306. The composition of any one of Embodiments 300-305, wherein the cells are or comprise NK cells expanded ex vivo.
307. The composition of any one of Embodiments 300-306, wherein the cells are or comprise memory-like NK cells.
308. The composition of any one of Embodiments 300-307, wherein the cells are or comprise cytokine-induced memory like NK cells.
309. The composition of any one of Embodiments 300-303, wherein the cells are or comprise NKT cells.
310. The composition of any one of Embodiments 300-303, wherein the cells are or comprise monocytes.
311. The composition of any one of Embodiments 300-303, wherein the cells are or comprise macrophages.
312. A pharmaceutical composition comprising an agent or composition of any one of the preceding Embodiments and a pharmaceutically acceptable carrier.
313. The composition of any one of Embodiments 300-312, wherein the composition comprises an immunoglobulin.
314. The composition of Embodiment 313, wherein the immunoglobulin is or comprises IgG.
315. The composition of any one of Embodiment 313-314, wherein the immunoglobulin is intravenous immunoglobulin.
316. The composition of any one of Embodiment 313-315, wherein the immunoglobulin is of an antibody toward a specific antigen.
317. The composition of any one of the preceding Embodiments, wherein the composition is cryopreserved.
318. A method for treating a CD38-associated condition, disorder or disease, comprising administering to a subject suffering therefrom an effective amount of an agent or composition of any one of the preceding Embodiments.
319. The method of Embodiment 318, comprising administering to a subject a population of cells.
320. The method of Embodiment 319, wherein the subject is subject to both the agent or composition and the population of cells.
321. The method of any one of Embodiments 319-320, wherein the cells are manipulated cells.
322. The method of any one of Embodiments 319-321, wherein the cells are manufactured cells.
323. The method of any one of Embodiments 319-322, wherein the cells are useful for a cell-based therapy.
324. The method of any one of Embodiments 319-323, wherein the cells are NK cells.
325. The method of any one of Embodiments 319-324, wherein the c, ells are engineered NK cells.
326. The method of any one of Embodiments 319-325, wherein the cells are NK cells expanded ex vivo.
327. The method of any one of Embodiments 319-326, wherein the cells are memory-like NK cells.
328. The method of any one of Embodiments 319-327, wherein the cells are cytokine-induced memory like NK cells.
329. The method of any one of Embodiments 319-323, wherein the cells are NKT cells.
330. The method of any one of Embodiments 319-323, wherein the cells are monocytes.
331. The method of any one of Embodiments 319-323, wherein the cells are macrophages.
332. The method of any one of Embodiments 319-331, wherein the cells are administered concurrently with the agent or composition.
333. The method of any one of Embodiments 319-332, wherein the cells are administered concurrently with the agent or composition in a composition comprising the cells and the agent or composition.
334. The method of any one of Embodiments 319-331, wherein the cells are administered prior to the administration of the agent or composition.
335. The method of any one of Embodiments 319-331, wherein the cells are administered subsequently to the administration of the agent or composition.
336. The method of any one of Embodiments 318-335, wherein the method comprises administration of an immunoglobulin.
337. The method of any one of Embodiments 319-335, wherein the method comprises administration of an immunoglobulin.
338. The method of any one of Embodiments 336-337, wherein the immunoglobulin is or comprises IgG.
339. The method of any one of Embodiment 336-338, wherein the immunoglobulin is intravenous immunoglobulin.
340. The method of any one of Embodiment 336-339, wherein the immunoglobulin is of an antibody toward a specific antigen.
341. The method of any one of Embodiments 336-340, wherein the immunoglobulin is administered concurrently with the agent or composition.
342. The method of any one of Embodiments 336-341, wherein the immunoglobulin is administered concurrently with the agent or composition in a composition comprising the immunoglobulin and the agent or composition.
343. The method of any one of Embodiments 336-340, wherein the immunoglobulin is administered prior or subsequently to with the agent or composition.
344. The method of any one of Embodiments 337-343, wherein the immunoglobulin is administered concurrently with the cells.
345. The method of any one of Embodiments 336-339, wherein the immunoglobulin is administered concurrently with the cells in a composition comprising the immunoglobulin and the cells.
346. The method of any one of Embodiments 336-339, wherein the immunoglobulin is administered prior or subsequently to with the cells.
347. The method of any one of the preceding Embodiments, wherein an administration of an agent is followed by one or more doses of cells of any one of the preceding Embodiments and/or one or more does of cells of any one of the preceding Embodiments and an agent of any one of the preceding Embodiments.
348. The method of Embodiment 347, wherein an administration of an agent is a single dose of an agent.
349. The method of any one of Embodiments 347-348, wherein an administration of an agent is followed by one or more doses of cells of any one of the preceding Embodiments.
350. The method of any one of Embodiments 347-349, wherein an administration of an agent is followed by one or more doses of cells of any one of the preceding Embodiments and an agent of any one of the preceding Embodiments.
351. The method of any one of Embodiments 347-350, wherein for each dose of cells and an agent, the cells are independently administered prior to, concurrently or subsequently to the agent.
352. The method of any one of Embodiments 347-350, wherein for at least one dose of cells and an agent, the cells are administered with the agent in the same composition.
353. A method, comprising:

a) providing a first compound comprising a target binding moiety as described in any one of the preceding Embodiments and a first reactive group;

b) providing a second compound comprising an antibody binding moiety as described in any one of the preceding Embodiments and a second reactive group; and

c) reacting the first reactive group with the second reactive group such that the target binding moiety and the antibody binding moiety are covalent linked.

354. The method of Embodiment 353, wherein the reaction between the first reactive group and the second reactive group is or comprises an amidation reaction.
355. The method of Embodiment 354, wherein one of the first reactive group and the second reactive group is or comprises

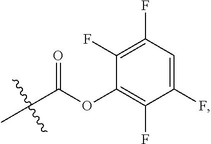 [SMILES: *C(=O)Oc1c(F)c(F)cc(F)c1F]

and the other is or comprise —NH₂.
356. The method of Embodiment 353, wherein the reaction between the first reactive group and the second reactive group is a cycloaddition reaction.
357. The method of any one of Embodiments 353-356, wherein the first compound is a compound of formula V or a salt thereof.
358. The method of any one of Embodiments 353-357, wherein the first compound is a compound of formula V or a salt thereof, wherein

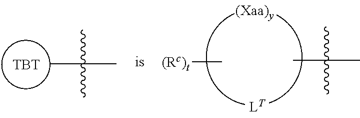

as described in any one of the preceding Embodiments.
359. The method of any one of Embodiments 353-358, wherein the second compound is a compound of formula IV, IV-a, IV-b, IV-c, or IV-d, or a salt thereof.
360. A method for manufacturing an agent or composition of any one of the preceding Embodiments, comprising reacting a first compound comprising an antibody binding moiety and an alkyne with a second compound comprising a target binding moiety and an azide, or reacting a first compound comprising an antibody binding moiety and an azide with a second compound comprising a target binding moiety and an alkyne.
361. The method of Embodiment 360, wherein the alkyne is a ring-stain activated alkyne.
362. The method of Embodiment 360, wherein the alkyne is an alkyne within a 8-membered ring.
363. The method of Embodiment 360, wherein the alkyne is an alkyne of a BCN moiety.
364. The method of any one of Embodiments 360-363, wherein the reaction is performed in the absence of a copper salt.
365. A method for recruiting an antibody to a target comprising or expressing CD38, comprising contacting the target with an agent or composition of any one of the preceding Embodiments.
366. A method for recruiting immune activity to a target comprising or expressing CD38, comprising contacting the target with an agent of any one of the preceding Embodiments.
367. The method of any one of Embodiments 365-366, wherein the target is a tumor cell.

### EXEMPLIFICATION

As depicted in the Examples below, in certain exemplary embodiments, compounds are prepared according to the following general procedures. It will be appreciated that, although the general methods depict the synthesis of certain compounds of the present disclosure, the following general methods, and other methods known to one of ordinary skill in the art, can be applied to all compounds and subclasses and species of each of these compounds, as described herein.

### Example 1. Exemplary Synthesis of Compounds

Exemplary Preparation of I-1.

Peptide Synthesis: Peptide were synthesized using standard Fmoc chemistry. An exemplary procedure is described below.

1) Add DCM to the vessel containing CTC Resin (0.300 mmol, 300 mg, 1.00 mmol/g) and Fmoc-Thr(tBu)-OH (95.3 mg, 0.240 mmol, 0.80 eq) with N₂ bubbling.
2) Add DIEA (4.00 eq) dropwise and mix for 2 hr.

### 3) Add MeOH (0.30 mL) and mix for 30 min.

4) Drain and wash with DMF for 5 times.
5) Add 20% piperidine/DMF and react for 30 min.
6) Drain and wash with DMF for 5 times.
7) Add Fmoc-amino acid solution and mix for 30 sec first, then add activation solution, and the coupling reaction lasts for 1 hr with continuous N₂ bubbling.
8) Repeat step 4 to 7 for next amino acid coupling.

| # | Materials | Coupling reagents |
|---|---|---|
| 1 | Fmoc-Thr(tBu)-OH (0.80 eq) | DIEA (4.00 eq) |
| 2 | Fmoc-Cys(Trt)-OH (3.00 eq) | HBTU(2.85 eq) |
| | | and DIEA (6.00 eq) |
| 3 | Fmoc-Trp(Boc)-OH (3.00 eq) | HBTU(2.85 eq) |
| | | and DIEA (6.00 eq) |
| 4 | Fmoc-Val-OH (3.00 eq) | HBTU(2.85 eq) |
| | | and DIEA (6.00 eq) |
| 5 | Fmoc-Leu-OH (3.00 eq) | HBTU(2.85 eq) |
| | | and DIEA (6.00 eq) |
| 6 | Fmoc-Glu(OtBu)-OH (3.00 eq) | HBTU(2.85 eq) |
| | | and DIEA (6.00 eq) |
| 7 | Fmoc-Gly-OH (3.00 eq) | HBTU(2.85 eq) |
| | | and DIEA (6.00 eq) |
| 8 | Fmoc-Leu-OH (3.00 eq) | HBTU(2.85 eq) |
| | | and DIEA (6.00 eq) |
| 9 | Fmoc-His(Trt)-OH (3.00 eq) | HATU(2.85 eq) |
| | | and DIEA (6.00 eq) |
| 10 | Fmoc-Trp(Boc)-OH (3.00 eq) | HATU(2.85 eq) |
| | | and DIEA (6.00 eq) |
| 11 | Fmoc-Ala-OH (3.00 eq) | HATU(2.85 eq) |
| | | and DIEA (6.00 eq) |
| 12 | Fmoc-Cys(Trt)-OH (3.00 eq) | HATU(2.85 eq) |
| | | and DIEA (6.00 eq) |
| 13 | Fmoc-Asp(OtBu)-OH (3.00 eq) | HATU(2.85 eq) |
| | | and DIEA (6.00 eq) |
| 14 | Fmoc-PEG6-CH₂CH₂—COOH | HATU(1.9 eq) |
| | (2.00 eq) | and DIEA (4.0 eq) |
| 15 | Fmoc-D-Arg(Pbf)- | HATU(2.85 eq) |
| | OH (3.00 eq) | and DIEA (6.0 eq) |
| 16 | 3-azidopropanoic acid | HATU(2.85 eq) |
| | (3.00 eq) | and DIEA (6.00 eq) |

In one preparation:
Synthesis scale: 0.24 mmol.
20% piperidine in DMF was used for Fmoc deprotection for 30 min.
The coupling reaction was monitored by ninhydrin test.
After last coupling, the resin was washed with MeOH for 3 times, and then dried under vacuum.

Peptide Cleavage and Purification. Various protocols may be utilized. In one example: Add cleavage cocktail (92.5% TFA/2.5% EDT/2.5% H₂O/2.5% TIS) to the flask containing the side chain protected peptide at room temperature and stir for 1.5 hr.

After filtration, the solution is added with cold isopropyl ether, and the peptide is precipitated collected by centrifugation (3 min at 3000 rpm).
The precipitate is washed with cold isopropyl ether for two additional times.
Dry the crude peptide under vacuum for 2 hr.
Dissolve the crude peptide in ACN/H₂O (1:1, 200 mL in total)
The disulfide bond is formed through I₂/MeOH, where the completion of the reaction is indicated by LCMS.
Lyophilize the reaction mixture to get the crude peptide.
Purify the crude peptide by prep-HPLC (A: 0.075% TFA in H₂O, B: ACN) to give the compound 2 (35.0 mg).

A mixture of compound 3 (5.0 mg, 1.0 eq) and compound 2 (4.55 mg, 1.0 eq) was dissolved in DMF (0.5 mL), and the reaction was stirred at 20° C. for 8 hr. LCMS showed the reaction was complete, and then the mixture was directly purified by prep-HPLC. Fractions with desired m/z (e.g., 1482.3, 1112.2, 890.1, 741.8, etc.) were combined and lyophilized to produce I-1 (4.1 mg, 42.9% yield, 95.4% purity) as a white solid. Purification conditions:

Separation condition

| | |
|---|---|
| Sample Preparation | Dissolve in DMF/H₂O |
| Instrument | Gilson GX-215 |
| Mobile Phase | A: H₂O (0.075% TFA in H₂O) |
| | B: CH₃CN |
| Gradient | 20-50%-60 min. Retention time: 56 min |
| Column | Luna C18, 25*200 mm, 10 um, 110A + Gemin |
| | C18, 150*30 mm, 5 um, 110A |
| Flow Rate | 20 mL/min |
| Wavelength | 220/254 nm |
| Oven Tem. | Room temperature |

Exemplary Preparation of I-2.

Exemplary Peptide Synthesis. Peptide were synthesized using standard Fmoc chemistry, for example:

1) Add DCM to the vessel containing CTC Resin (0.300 mmol, 300 mg, 1.00 mmol/g) and Fmoc-Thr(tBu)-OH (95.3 mg, 0.240 mmol, 0.80 eq) with N₂ bubbling.
2) Add DIEA (4.00 eq) dropwise and mix for 2 hr.

### 3) Add MeOH (0.30 mL) and mix for 30 min.

4) Drain and wash with DMF for 5 times.
5) Add 20% piperidine/DMF and react for 30 min.
6) Drain and wash with DMF for 5 times.
7) Add Fmoc-amino acid solution and mix for 30 sec first, then add activation solution, and the coupling reaction lasts for 1 hr with continuous N₂ bubbling.
8) Repeat step 4 to 7 for next amino acid coupling.

| # | Materials | Coupling reagents |
|---|---|---|
| 1 | Fmoc-Thr(tBu)-OH (0.80 eq) | DIEA (4.00 eq) |
| 2 | Fmoc-Cys(Trt)-OH (3.00 eq) | HBTU(2.85 eq) |
| | | and DIEA (6.00 eq) |
| 3 | Fmoc-Trp(Boc)-OH (3.00 eq) | HBTU(2.85 eq) |
| | | and DIEA (6.00 eq) |
| 4 | Fmoc-Val-OH (3.00 eq) | HBTU(2.85 eq) |
| | | and DIEA (6.00 eq) |
| 5 | Fmoc-Leu-OH (3.00 eq) | HBTU(2.85 eq) |
| | | and DIEA (6.00 eq) |
| 6 | Fmoc-Glu(OtBu)-OH (3.00 eq) | HBTU(2.85 eq) |
| | | and DIEA (6.00 eq) |
| 7 | Fmoc-Gly-OH (3.00 eq) | HBTU(2.85 eq) |
| | | and DIEA (6.00 eq) |
| 8 | Fmoc-Leu-OH (3.00 eq) | HBTU(2.85 eq) |
| | | and DIEA (6.00 eq) |
| 9 | Fmoc-His(Trt)-OH (3.00 eq) | HATU(2.85 eq) |
| | | and DIEA (6.00 eq) |
| 10 | Fmoc-Trp(Boc)-OH (3.00 eq) | HATU(2.85 eq) |
| | | and DIEA (6.00 eq) |
| 11 | Fmoc-Ala-OH (3.00 eq) | HATU(2.85 eq) |
| | | and DIEA (6.00 eq) |
| 12 | Fmoc-Cys(Trt)-OH (3.00 eq) | HATU(2.85 eq) |
| | | and DIEA (6.00 eq) |
| 13 | Fmoc-Asp(OtBu)-OH (3.00 eq) | HATU(2.85 eq) |
| | | and DIEA (6.00 eq) |
| 14 | Fmoc-PEG6-CH₂CH₂—COOH | HATU(1.9 eq) |
| | (2.00 eq) | and DIEA (4.0 eq) |
| 15 | Fmoc-D-Arg(Pbf)-OH (3.00 eq) | HATU(2.85 eq) |
| | | and DIEA (6.0 eq) |

Synthesis scale: 0.24 mmol.
20% piperidine in DMF was used for Fmoc deprotection for 30 min.
The coupling reaction was monitored by ninhydrin test.
After last amino acid coupling, N-terminal Fmoc was removed, and the resin was washed with MeOH for 3 times, and then dried under vacuum.

### Peptide Cleavage and Purification:

Add cleavage cocktail (92.5% TFA/2.5% EDT/2.5% H₂O/2.5% TIS) to the flask containing the side chain protected peptide at room temperature and stir for 1.5 hr.
After filtration, the solution is added with cold isopropyl ether, and the peptide is precipitated collected by centrifugation (3 min at 3000 rpm).
The precipitate is washed with cold isopropyl ether for two additional times.
Dry the crude peptide under vacuum for 2 hr.
Dissolve the crude peptide in ACN/H₂O (1:1, 200 mL in total)
The disulfide bond is formed through I₂/MeOH, where the completion of the reaction is indicated by LCMS.
Lyophilize the reaction mixture to get the crude peptide.
Purify the crude peptide by prep-HPLC (A: 0.075% TFA in H₂O, B: ACN) to give the compound 2 (35.0 mg).

A mixture of compound 2 (35.0 mg, 1.0 eq) and DBCO-NHS (7.66 mg, 1.1 eq) was dissolved in DMF (1.0 mL), and then DIEA (6.00 eq) was added slowly. The mixture was stirred at 20° C. for 8 hr. LCMS showed the reaction was complete. The mixture was then directly purified by prep-HPLC (TFA condition), and compound 3 (11.0 mg, 32.4% yield) was obtained as a white solid.

Compound 4 was prepared by reacting a corresponding peptide and 3-azidopropanoic-NHS (0.97 mg, 1.1 eq), which were dissolved in DMF (0.5 mL). Then DIEA (6.0 eq) was added slowly. The mixture was stirred at 20° C. for 2 hr. LCMS showed the reaction was complete. The mixture was then directly purified by prep-HPLC (TFA condition), and compound 4 (5.50 mg, 52.4% yield) was obtained as a white solid.

A mixture of compound 4 (5.5 mg, 1.0 eq) and compound 3 (5.10 mg, 1.0 eq) was dissolved in DMF (0.5 mL), and the reaction was stirred at 20° C. for 8 hr. LCMS showed the reaction was complete, and then the mixture was directly purified by prep-HPLC. Fractions with desired m/z (e.g., 1600.3, 1200.4, 960.5, etc.) were combined and lyophilized to produce I-2 (4.1 mg, 38.7% yield, 95.7% purity) as a white solid. Purification conditions:

Separation condition

| | |
|---|---|
| Sample Preparation | Dissolve in DMF/H₂O |
| Instrument | Gilson GX-215 |
| Mobile Phase | A: H₂O (0.075% TFA in H₂O) |
| | B: CH₃CN |
| Gradient | 20-50%-60 min. Retention time: 56 min |
| Column | Luna C18, 25*200 mm, 10 um, 110A + |
| | Gemin C18, 150*30 mm, 5 um, 110A |
| Flow Rate | 20 mL/min |
| Wavelength | 220/254 nm |
| Oven Tem. | Room temperature |

Exemplary Preparation of I-3.

Exemplary Peptide Synthesis. Peptide were synthesized using standard Fmoc chemistry, for example:

1) Add DCM to the vessel containing CTC Resin (0.300 mmol, 300 mg, 1.00 mmol/g) and Fmoc-Thr(tBu)-OH (95.3 mg, 0.240 mmol, 0.80 eq) with N₂ bubbling.
2) Add DIEA (4.00 eq) dropwise and mix for 2 hr.

### 3) Add MeOH (0.30 mL) and mix for 30 min.

4) Drain and wash with DMF for 5 times.
5) Add 20% piperidine/DMF and react for 30 min.
6) Drain and wash with DMF for 5 times.
7) Add Fmoc-amino acid solution and mix for 30 sec first, then add activation solution, and the coupling reaction lasts for 1 hr with continuous N₂ bubbling.
8) Repeat step 4 to 7 for next amino acid coupling.

| # | Materials | Coupling reagents |
|---|---|---|
| 1 | Fmoc-Thr(tBu)-OH (0.80 eq) | DIEA (4.00 eq) |
| 2 | Fmoc-Cys(Trt)-OH (3.00 eq) | HBTU(2.85 eq) |
| | | and DIEA (6.00 eq) |
| 3 | Fmoc-Trp(Boc)-OH (3.00 eq) | HBTU(2.85 eq) |
| | | and DIEA (6.00 eq) |
| 4 | Fmoc-Val-OH (3.00 eq) | HBTU(2.85 eq) |
| | | and DIEA (6.00 eq) |
| 5 | Fmoc-Leu-OH (3.00 eq) | HBTU(2.85 eq) |
| | | and DIEA (6.00 eq) |
| 6 | Fmoc-Glu(OtBu)-OH (3.00 eq) | HBTU(2.85 eq) |
| | | and DIEA (6.00 eq) |
| 7 | Fmoc-Gly-OH (3.00 eq) | HBTU(2.85 eq) |
| | | and DIEA (6.00 eq) |
| 8 | Fmoc-Leu-OH (3.00 eq) | HBTU(2.85 eq) |
| | | and DIEA (6.00 eq) |
| 9 | Fmoc-His(Trt)-OH (3.00 eq) | HATU(2.85 eq) |
| | | and DIEA (6.00 eq) |
| 10 | Fmoc-Trp(Boc)-OH (3.00 eq) | HATU(2.85 eq) |
| | | and DIEA (6.00 eq) |
| 11 | Fmoc-Ala-OH (3.00 eq) | HATU(2.85 eq) |
| | | and DIEA (6.00 eq) |
| 12 | Fmoc-Cys(Trt)-OH (3.00 eq) | HATU(2.85 eq) |
| | | and DIEA (6.00 eq) |
| 13 | Fmoc-Asp(OtBu)-OH (3.00 eq) | HATU(2.85 eq) |
| | | and DIEA (6.00 eq) |
| 14 | Fmoc-PEG6-CH₂CH₂—COOH | HATU(1.9 eq) |
| | (2.00 eq) | and DIEA (4.0 eq) |
| 15 | Fmoc-D-Arg(Pbf)-OH | HATU(2.85 eq) |
| | (3.00 eq) | and DIEA (6.0 eq) |

Synthesis scale: 0.24 mmol.
20% piperidine in DMF was used for Fmoc deprotection for 30 min.
The coupling reaction was monitored by ninhydrin test.
After last amino acid coupling, N-terminal Fmoc was removed, and the resin was washed with MeOH for 3 times, and then dried under vacuum.

### Peptide Cleavage and Purification:

Add cleavage cocktail (92.5% TFA/2.5% EDT/2.5% H₂O/2.5% TIS) to the flask containing the side chain protected peptide at room temperature and stir for 1.5 hr.
After filtration, the solution is added with cold isopropyl ether, and the peptide is precipitated collected by centrifugation (3 min at 3000 rpm).
The precipitate is washed with cold isopropyl ether for two additional times.
Dry the crude peptide under vacuum for 2 hr.
Dissolve the crude peptide in ACN/H₂O (1:1, 200 mL in total)
The disulfide bond is formed through I₂/MeOH, where the completion of the reaction is indicated by LCMS.
Lyophilize the reaction mixture to get the crude peptide.
Purify the crude peptide by prep-HPLC (A: 0.075% TFA in H₂O, B: ACN) to give the compound 2 (45.0 mg).

A mixture of compound 2 (45.0 mg, 1.0 eq) and BCN-NHS (6.48 mg, 1.1 eq) was dissolved in DMF (1.0 mL), and then DIEA (6.0 eq) was added slowly. The mixture was stirred at 20° C. for 8 hr. LCMS showed the reaction was complete. The mixture was then directly purified by prep-HPLC (TFA condition), and compound 3 (14.0 mg, 28.6% yield) was obtained as a white solid.

A mixture of compound 3 (14.0 mg, 1.0 eq) and compound 75 (12.7 mg, 1.0 eq) was dissolved in DMF (1.0 mL), and the reaction was stirred at 20° C. for 8 hr. LCMS showed the reaction was complete, and then the mixture was directly purified by prep-HPLC. Fractions with desired m/z (e.g., 1049.9, 840.0, 700.4, etc.) were combined and lyophilized to produce I-3 (11.1 mg, 41.6% yield, 97.1% purity) as a white solid. Purification conditions:

Separation condition

| | |
|---|---|
| Sample Preparation | Dissolve in DMF/H₂O |
| Instrument | Gilson GX-215 |
| Mobile Phase | A: H₂O (0.075% TFA in H₂O) |
| | B: CH₃CN |
| Gradient | 20-50%-60 min. Retention time: 49 min |
| Column | Luna C18, 25*200 mm, 10 um, 110A + |
| | Gemin C18, 150*30 mm, 5 um, 110A |
| Flow Rate | 20 mL/min |
| Wavelength | 220/254 nm |
| Oven Tem. | Room temperature |

Exemplary Preparation of I-4.

Exemplary Peptide Synthesis. Peptide were synthesized using standard Fmoc chemistry, for example:

1) Add DCM to the vessel containing CTC Resin (0.300 mmol, 300 mg, 1.00 mmol/g) and Fmoc-Thr(tBu)-OH (95.3 mg, 0.240 mmol, 0.80 eq) with N₂ bubbling.
2) Add DIEA (4.00 eq) dropwise and mix for 2 hr.

### 3) Add MeOH (0.30 mL) and mix for 30 min.

4) Drain and wash with DMF for 5 times.
5) Add 20% piperidine/DMF and react for 30 min.
6) Drain and wash with DMF for 5 times.
7) Add Fmoc-amino acid solution and mix for 30 sec first, then add activation solution, and the coupling reaction lasts for 1 hr with continuous N₂ bubbling.
8) Repeat step 4 to 7 for next amino acid coupling.

| # | Materials | Coupling reagents |
|---|---|---|
| 1 | Fmoc-Thr(tBu)-OH (0.80 eq) | DIEA (4.00 eq) |
| 2 | Fmoc-Cys(Trt)-OH (3.00 eq) | HBTU(2.85 eq) |
| | | and DIEA (6.00 eq) |
| 3 | Fmoc-Trp(Boc)-OH (3.00 eq) | HBTU(2.85 eq) |
| | | and DIEA (6.00 eq) |
| 4 | Fmoc-Val-OH (3.00 eq) | HBTU(2.85 eq) |
| | | and DIEA (6.00 eq) |
| 5 | Fmoc-Leu-OH (3.00 eq) | HBTU(2.85 eq) |
| | | and DIEA (6.00 eq) |
| 6 | Fmoc-Glu(OtBu)-OH (3.00 eq) | HBTU(2.85 eq) |
| | | and DIEA (6.00 eq) |
| 7 | Fmoc-Gly-OH (3.00 eq) | HBTU(2.85 eq) |
| | | and DIEA (6.00 eq) |
| 8 | Fmoc-Leu-OH (3.00 eq) | HBTU(2.85 eq) |
| | | and DIEA (6.00 eq) |
| 9 | Fmoc-His(Trt)-OH (3.00 eq) | HATU(2.85 eq) |
| | | and DIEA (6.00 eq) |
| 10 | Fmoc-Trp(Boc)-OH (3.00 eq) | HATU(2.85 eq) |
| | | and DIEA (6.00 eq) |
| 11 | Fmoc-Ala-OH (3.00 eq) | HATU(2.85 eq) |
| | | and DIEA (6.00 eq) |
| 12 | Fmoc-Cys(Trt)-OH (3.00 eq) | HATU(2.85 eq) |
| | | and DIEA (6.00 eq) |
| 13 | Fmoc-Asp(OtBu)-OH (3.00 eq) | HATU(2.85 eq) |
| | | and DIEA (6.00 eq) |
| 14 | Fmoc-PEG8-CH₂CH₂—COOH | HATU(1.9 eq) |
| | (2.00 eq) | and DIEA (4.0 eq) |
| 15 | Fmoc-D-Arg(Pbf)-OH (3.00 eq) | HATU(2.85 eq) |
| | | and DIEA (6.0 eq) |
| 16 | Fmoc-D-Arg(Pbf)-OH (3.00 eq) | HATU(2.85 eq) |
| | | and DIEA (6.0 eq) |

Synthesis scale: 0.24 mmol
20% piperidine in DMF was used for Fmoc deprotection for 30 min.
The coupling reaction was monitored by ninhydrin test.
After last amino acid coupling, N-terminal Fmoc was removed, and the resin was washed with MeOH for 3 times, and then dried under vacuum.

### Peptide Cleavage and Purification:

Add cleavage cocktail (92.5% TFA/2.5% EDT/2.5% H₂O/2.5% TIS) to the flask containing the side chain protected peptide at room temperature and stir for 1.5 hr.
After filtration, the solution is added with cold isopropyl ether, and the peptide is precipitated collected by centrifugation (3 min at 3000 rpm).
The precipitate is washed with cold isopropyl ether for two additional times.
Dry the crude peptide under vacuum for 2 hr.
Dissolve the crude peptide in ACN/H₂O (1:1, 200 mL in total)
The disulfide bond is formed through I₂/MeOH, where the completion of the reaction is indicated by LCMS.
Lyophilize the reaction mixture to get the crude peptide.
Purify the crude peptide by prep-HPLC (A: 0.075% TFA in H₂O, B: ACN) to give the compound 2 (48.0 mg).

A mixture of compound 2 (48.0 mg, 1.0 eq) and BCN-NHS (9.37 mg, 1.1 eq) was dissolved in DMF (1.0 mL), and then DIEA (6.0 eq) was added slowly. The mixture was stirred at 20° C. for 8 hr. LCMS showed the reaction was complete. The mixture was then directly purified by prep-HPLC (TFA condition), and compound 3 (14.0 mg, 28.6% yield) was obtained as a white solid.

A mixture of compound 3 (14.0 mg, 1.0 eq) and compound 75 (11.0 mg, 1.0 eq) was dissolved in DMF (1.0 mL), and the reaction was stirred at 20° C. for 8 hr. LCMS showed the reaction was complete, and then the mixture was directly purified by prep-HPLC. Fractions with desired m/z (e.g., 1138.6, 911.2, 759.4, etc.) were combined and lyophilized to produce I-4 (15.9 mg, 63.3% yield, 96.6% purity) as a white solid. Purification conditions:

Separation condition

| | |
|---|---|
| Sample Preparation | Dissolve in DMF/H₂O |
| Instrument | Gilson GX-215 |
| Mobile Phase | A: H₂O (0.075% TFA in H₂O) |
| | B: CH₃CN |
| Gradient | 20-50%-60 min. Retention time: 52 min |
| Column | Luna C18, 25*200 mm, 10 um, 110A + |
| | Gemin C18, 150*30 mm, 5 um, 110A |
| Flow Rate | 20 mL/min |
| Wavelength | 220/254 nm |
| Oven Tem. | Room temperature |

Exemplary Preparation of I-5.

Exemplary Peptide Synthesis. Peptide were synthesized using standard Fmoc chemistry, for example:

1) Add DCM to the vessel containing CTC Resin (0.300 mmol, 300 mg, 1.00 mmol/g) and Fmoc-Thr(tBu)-OH (95.3 mg, 0.240 mmol, 0.80 eq) with N₂ bubbling.
2) Add DIEA (4.00 eq) dropwise and mix for 2 hours.

### 3) Add MeOH (0.30 mL) and mix for 30 min.

4) Drain and wash with DMF for 5 times.
5) Add 20% piperidine/DMF and react on 30 min.
6) Drain and wash with DMF for 5 times.
7) Add Fmoc-amino acid solution and mix for 30 sec first, then add activation solution, and the coupling reaction lasts for 1 hr with continuous N₂ bubbling.
8) Repeat step 4 to 7 for next amino acid coupling.

| # | Materials | Coupling reagents |
|---|---|---|
| 1 | Fmoc-Thr(tBu)-OH (0.80 eq) | DIEA (4.00 eq) |
| 2 | Fmoc-Cys(Trt)-OH (3.00 eq) | HBTU(2.85 eq) |
| | | and DIEA (6.00 eq) |
| 3 | Fmoc-Trp(Boc)-OH (3.00 eq) | HBTU(2.85 eq) |
| | | and DIEA (6.00 eq) |
| 4 | Fmoc-Val-OH (3.00 eq) | HBTU(2.85 eq) |
| | | and DIEA (6.00 eq) |
| 5 | Fmoc-Leu-OH (3.00 eq) | HBTU(2.85 eq) |
| | | and DIEA (6.00 eq) |
| 6 | Fmoc-Glu(OtBu)-OH (3.00 eq) | HBTU(2.85 eq) |
| | | and DIEA (6.00 eq) |
| 7 | Fmoc-Gly-OH (3.00 eq) | HBTU(2.85 eq) |
| | | and DIEA (6.00 eq) |
| 8 | Fmoc-Leu-OH (3.00 eq) | HBTU(2.85 eq) |
| | | and DIEA (6.00 eq) |
| 9 | Fmoc-His(Trt)-OH (3.00 eq) | HATU(2.85 eq) |
| | | and DIEA (6.00 eq) |
| 10 | Fmoc-Trp(Boc)-OH (3.00 eq) | HATU(2.85 eq) |
| | | and DIEA (6.00 eq) |
| 11 | Fmoc-Ala-OH (3.00 eq) | HATU(2.85 eq) |
| | | and DIEA (6.00 eq) |
| 12 | Fmoc-Cys(Trt)-OH (3.00 eq) | HATU(2.85 eq) |
| | | and DIEA (6.00 eq) |
| 13 | Fmoc-Asp(OtBu)-OH (3.00 eq) | HATU(2.85 eq) |
| | | and DIEA (6.00 eq) |
| 14 | Fmoc-PEG3-CH₂CH₂—COOH | HATU(1.9 eq) |
| | (2.00 eq) | and DIEA (4.0 eq) |
| 15 | Fmoc-D-Arg(Pbf)-OH (3.00 eq) | HATU(2.85 eq) |
| | | and DIEA (6.0 eq) |

Synthesis scale: 0.24 mmol
20% piperidine in DMF was used for Fmoc deprotection for 30 min.
The coupling reaction was monitored by ninhydrin test.
After last amino acid coupling, N-terminal Fmoc was removed, and the resin was washed with MeOH for 3 times, and then dried under vacuum.

### Peptide Cleavage and Purification:

Add cleavage cocktail (92.5% TFA/2.5% EDT/2.5% H₂O/2.5% TIS) to the flask containing the side chain protected peptide at room temperature and stir for 1.5 hr.
After filtration, the solution is added with cold isopropyl ether, and the peptide is precipitated collected by centrifugation (3 min at 3000 rpm).
The precipitate is washed with cold isopropyl ether for two additional times.
Dry the crude peptide under vacuum for 2 hr.
Dissolve the crude peptide in ACN/H₂O (1:1, 200 mL in total)
The disulfide bond is formed through I₂/MeOH, where the completion of the reaction is indicated by LCMS.
Lyophilize the reaction mixture to get the crude peptide.
Purify the crude peptide by prep-HPLC (A: 0.075% TFA in H₂O, B: ACN) to give the compound 2 (42.0 mg).

A mixture of compound 2 (42.0 mg, 1.0 eq) and BCN-NHS (7.11 mg, 1.1 eq) was dissolved in DMF (1.0 mL), and then DIEA (6.0 eq) was added slowly. The mixture was stirred at 20° C. for 8 hr. LCMS showed the reaction was complete. The mixture was then directly purified by prep-HPLC (TFA condition), and compound 3 (15.0 mg, 32.7% yield) was obtained as a white solid.

A mixture of compound 4 (13.5 mg, 1.0 eq) and compound 3 (15.0 mg, 1.0 eq) was dissolved in DMF (1.0 mL), and the reaction was stirred at 20° C. for 8 hr. LCMS showed the reaction was complete, and then the mixture was directly purified by prep-HPLC. Fractions with desired m/z (e.g., 983.8, 787.6, etc.) were combined and lyophilized to produce I-5 (10.5 mg, 36.8% yield, 95.1% purity) as a white solid.

### Purification Conditions:

Separation condition

| | |
|---|---|
| Sample Preparation | Dissolve in DMF/H₂O |
| Instrument | Gilson GX-215 |
| Mobile Phase | A: H₂O (0.075% TFA in H₂O) |
| | B: CH₃CN |
| Gradient | 20-50%-60 min. Retention time: 48 min |
| Column | Luna C18, 25*200 mm, 10 um, 110A + |
| | Gemin C18, 150*30 mm, 5 um, 110A |
| Flow Rate | 20 mL/min |
| Wavelength | 220/254 nm |
| Oven Tem. | Room temperature |

Exemplary Preparation of I-6.

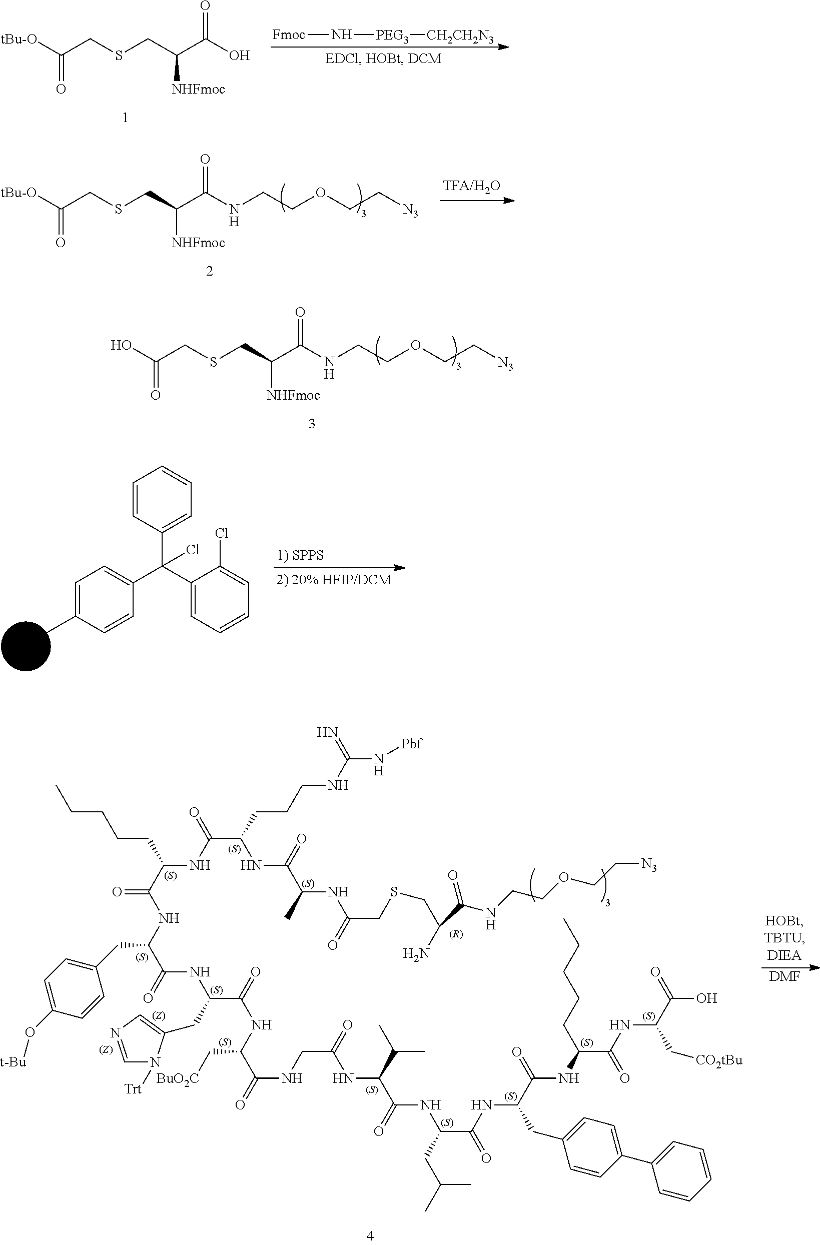

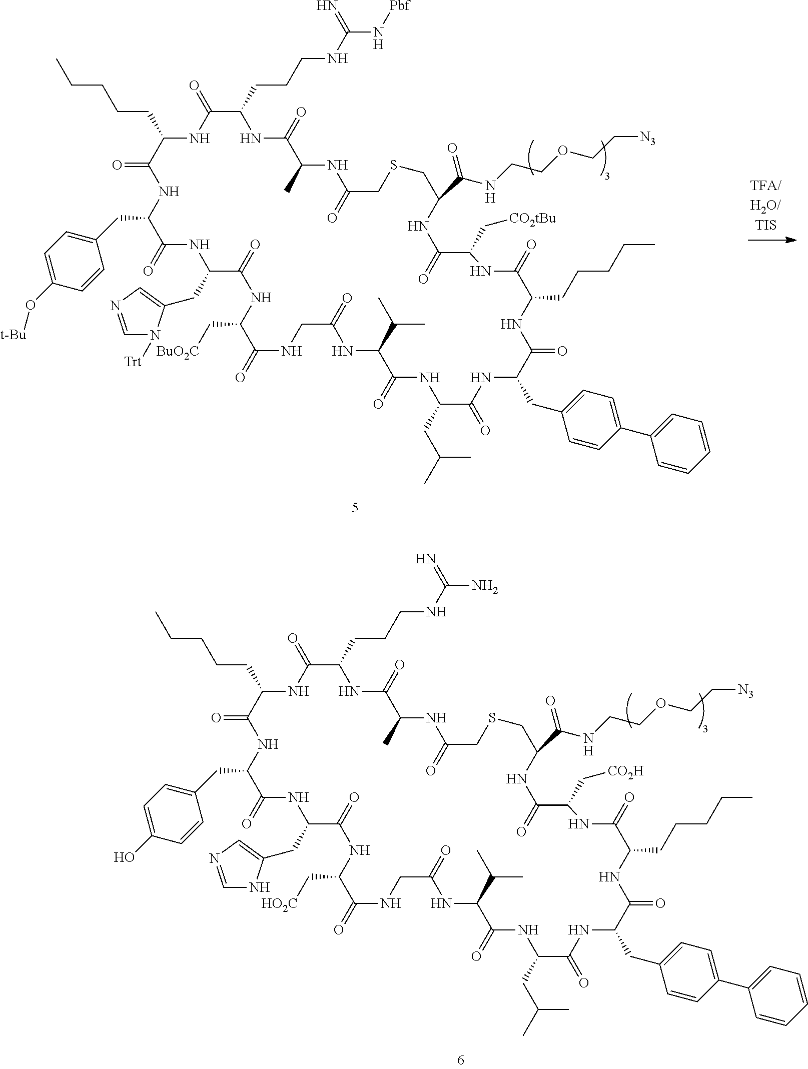

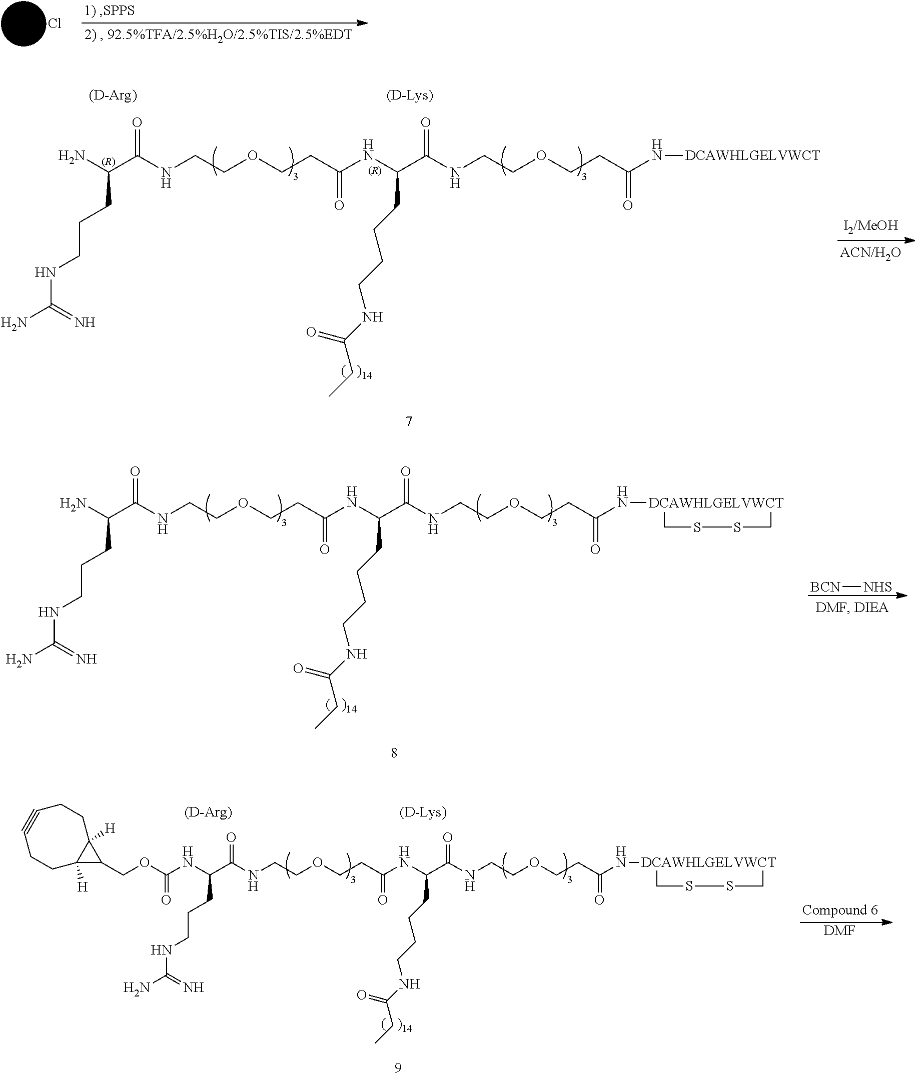

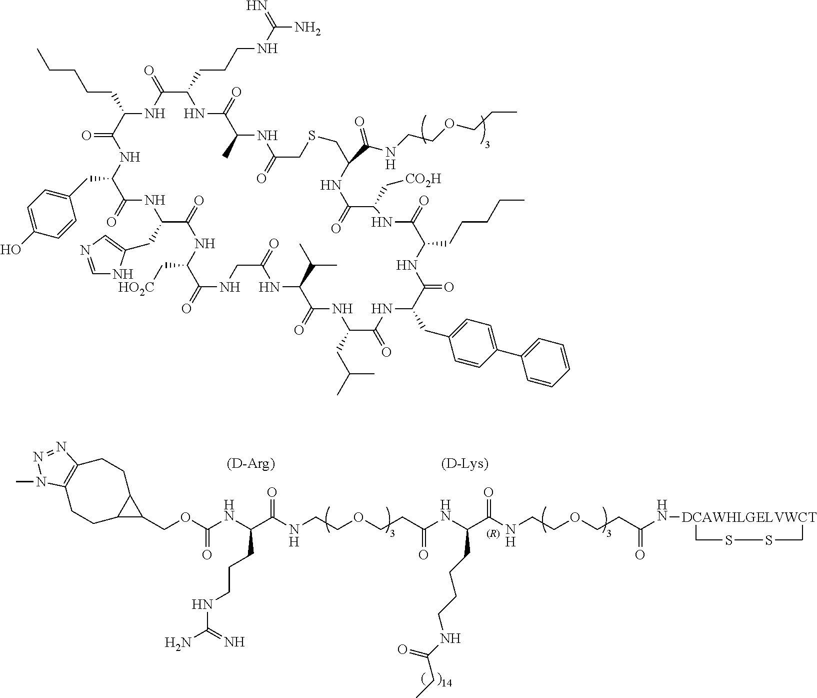

A mixture of compound 1 (2.00 g, 4.37 mmol), Fmoc-NH-PEG₃-CH₂CH₂N₃ (1.00 g, 4.59 mmol), EDCI (1.68 g, 8.74 mmol), HOBt (1.18 g, 8.74 mmol) was dissolved in DCM (20.0 mL), and the reaction was stirred at 15° C. for 16 hr. The solution was then diluted with DCM (100 mL), washed with 1 M HCl (30 mL), H₂O (30 mL), brine (30 mL), dried over anhydrous Na₂SO₄, concentrated under reduced pressure to get compound 2 (3.00 g, crude) as colorless oil.

Compound 2 (3.00 g, crude) was treated with TFA/H₂O (95/5, 20 mL in total) for 1 hr at 15° C. The solvent was then removed under reduced pressure, and the resulting residue was purified by flash C18 chromatography (ISCO®; 120 g SepaFlash® C18 Flash Column, Eluent of 0˜100% MeCN/H₂O @ 75 mL/min) to get compound 3 (1.50 g, 2.24 mmol, 49.2% yield) as a white solid.

Exemplary Peptide Synthesis. Peptide were synthesized using standard Fmoc chemistry, for example:

1) Add DCM (10.0 mL) to the vessel containing CTC Resin (2.0 mmol, 2.0 g, 1.0 mmol/g) and Fmoc-Asp(OtBu)-OH (658 mg, 1.60 mmol, 0.80 eq) with N₂ bubbling.
2) Add DIEA (4.00 eq) dropwise and mix for 2 hr.

### 3) Add MeOH (2.0 mL) and mix for 30 min.

4) Drain and wash with DMF for 5 times.
5) Add 20% piperidine/DMF and react for 30 min.
6) Drain and wash with DMF for 5 times.
7) Add Fmoc-amino acid solution and mix for 30 sec first, then add activation buffer, and the coupling reaction lasts for 1 hr with continuous N₂ bubbling.
8) Repeat step 4 to 7 for next amino acid coupling.

| Step | Materials | Coupling reagents |
|---|---|---|
| 1 | Fmoc-Asp(OtBu)-OH (0.80 eq) | DIEA (4.00 eq) |
| 2 | Fmoc-HomoNle-OH (2.00 eq) | HATU (1.90 eq) |
| | | and DIEA (4.00 eq) |
| 3 | Fmoc-Bip-OH (2.00 eq) | HATU (1.90 eq) |
| | | and DIEA (4.00 eq) |
| 4 | Fmoc-Leu-OH (3.00 eq) | HBTU (2.85 eq) |
| | | and DIEA (6.00 eq) |
| 5 | Fmoc-Val-OH (3.00 eq) | HBTU (2.85 eq) |
| | | and DIEA (6.00 eq) |
| 6 | Fmoc-Gly-OH (3.00 eq) | HBTU (2.85 eq) |
| | | and DIEA (6.00 eq) |
| 7 | Fmoc-Asp(OtBu)-OH (3.00 eq) | HBTU (2.85 eq) |
| | | and DIEA (6.00 eq) |
| 8 | Fmoc-His(Trt)-OH (3.00 eq) | HATU (2.85 eq) |
| | | and DIEA (6.00 eq) |
| 9 | Fmoc-Tyr(tBu)-OH (3.00 eq) | HATU (2.85 eq) |
| | | and DIEA (6.00 eq) |
| 10 | Fmoc-HomoNle-OH (2.00 eq) | HATU (1.90 eq) |
| | | and DIEA (4.00 eq) |
| 11 | Fmoc-Arg(Pbf)-OH (3.00 eq) | HATU (2.85 eq) |
| | | and DIEA (6.00 eq) |
| 12 | Fmoc-Ala-OH (3.00 eq) | HATU (2.85 eq) |
| | | and DIEA (6.00 eq) |
| 13 | Compound 3 (2.00 eq) | HATU (1.90 eq) |
| | | and DIEA (4.00 eq) |

Synthesis scale: 1.6 mmol
20% piperidine in DMF was used for Fmoc deprotection for 30 min.
The coupling reaction was monitored by ninhydrin test.
After coupling, the Fmoc group was removed, and the resin was washed with MeOH for 3 times, and then dried under vacuum.

### Peptide Cleavage and Purification:

Add cleavage solution (20% HFIP/DCM, 80 mL) to the flask containing the side chain protected peptide at room temperature. The cleavage was carried out twice (30 min each), with continuous N₂ bubbling.
After filtered, the filtrate was concentrated under reduced pressure and the residue was dried in lyophilizer to give compound 4 (2.9 g, crude) as a white solid.
A solution of compound 4 (2.9 g, 1.0 eq), HOBt (308 mg, 2.0 eq), TBTU (732 mg, 2.0 eq), DIEA (0.85 mL, 4.0 eq) in DMF (800 mL) was stirred at 15° C. for 1 hr. When cyclization was complete, the solution was then diluted with EA (1.5 L), washed with 1 M HCl (600 mL), brine (400 mL×4), dried over anhydrous Na₂SO₄, concentrated under reduced pressure to get compound 5 (3.2 g, crude) as colorless oil. Deprotection of crude cyclic peptide was carried out by treating compound 5 (3.2 g, crude) with TFA/TIS/H2O (95/2.5/2.5, 40 mL in total) by continuous stirring for 1.5 hr at 15° C. The solution was triturated with cold isopropyl ether (500 mL) and the precipitate was collected by centrifugation (3 min at 3000 rpm). The precipitate (deprotected peptide) was washed twice with isopropyl ether (50 mL each), following by drying under vacuum for 2 hr. The residue was purified by prep-HPLC (acidic condition, TFA) to give the compound 6 (600 mg).

Another example:

1) Add DCM to the vessel containing CTC Resin (0.500 mmol, 500 mg, 1.00 mmol/g) and Fmoc-Thr(tBu)-OH (159 mg, 0.400 mmol, 0.80 eq) with N₂ bubbling.
2) Add DIEA (4.00 eq) dropwise and mix for 2 hr.

### 3) Add MeOH (0.50 mL) and mix for 30 min.

4) Drain and wash with DMF for 5 times.
5) Add 20% piperidine/DMF and react for 30 min.
6) Drain and wash with DMF for 5 times.
7) Add Fmoc-amino acid solution and mix for 30 sec first, then add activation solution, and the coupling reaction lasts for 1 hr with continuous N₂ bubbling.
8) Repeat step 4 to 7 for next amino acid coupling.

| # | Materials | Coupling reagents |
|---|---|---|
| 1 | Fmoc-Thr(tBu)-OH (0.80 eq) | DIEA (4.00 eq) |
| 2 | Fmoc-Cys(Trt)-OH (3.00 eq) | HBTU(2.85 eq) |
| | | and DIEA (6.00 eq) |
| 3 | Fmoc-Trp(Boc)-OH (3.00 eq) | HBTU(2.85 eq) |
| | | and DIEA (6.00 eq) |
| 4 | Fmoc-Val-OH (3.00 eq) | HBTU(2.85 eq) |
| | | and DIEA (6.00 eq) |
| 5 | Fmoc-Leu-OH (3.00 eq) | HBTU(2.85 eq) |
| | | and DIEA (6.00 eq) |
| 6 | Fmoc-Glu(OtBu)-OH (3.00 eq) | HBTU(2.85 eq) |
| | | and DIEA (6.00 eq) |
| 7 | Fmoc-Gly-OH (3.00 eq) | HBTU(2.85 eq) |
| | | and DIEA (6.00 eq) |
| 8 | Fmoc-Leu-OH (3.00 eq) | HBTU(2.85 eq) |
| | | and DIEA (6.00 eq) |
| 9 | Fmoc-His(Trt)-OH (3.00 eq) | HATU(2.85 eq) |
| | | and DIEA (6.00 eq) |
| 10 | Fmoc-Trp(Boc)-OH (3.00 eq) | HATU(2.85 eq) |
| | | and DIEA (6.00 eq) |
| 11 | Fmoc-Ala-OH (3.00 eq) | HATU(2.85 eq) |
| | | and DIEA (6.00 eq) |
| 12 | Fmoc-Cys(Trt)-OH (3.00 eq) | HATU(2.85 eq) |
| | | and DIEA (6.00 eq) |
| 13 | Fmoc-Asp(OtBu)-OH (3.00 eq) | HATU(2.85 eq) |
| | | and DIEA (6.00 eq) |
| 14 | Fmoc-PEG3-CH₂CH₂COOH | HATU(1.90 eq) |
| | (2.00 eq) | and DIEA (4.0 eq) |
| 15 | Fmoc-D-Lys(Dde)-OH (2.00 eq) | HATU(1.90 eq) |
| | | and DIEA (4.0 eq) |
| 16 | Fmoc-PEG3-CH₂CH₂COOH | HATU(1.90 eq) |
| | (2.00 eq) | and DIEA (4.0 eq) |
| 17 | Boc-D-Arg(Pbf)-OH (3.00 eq) | HATU(2.85 eq) |
| | | and DIEA (6.00 eq) |
| 18 | Palmitic acid (3.00 eq) | HBTU(2.85 eq) |
| | | and DIEA (6.00 eq) |

Synthesis scale: 0.4 mmol
20% piperidine in DMF was used for Fmoc deprotection for 30 min.
The coupling reaction was monitored by ninhydrin test.
3% NH₂NH₂H₂O/DMF was used for De-Dde after cycle 17, and then palmitic acid was coupled to the sidechain of D-Lys.
After last coupling, the resin was washed with MeOH for 3 times, and then dried under vacuum.

### Peptide Cleavage and Purification:

Add cleavage cocktail (92.5% TFA/2.5% EDT/2.5% H₂O/2.5% TIS) to the flask containing the side chain protected peptide at room temperature and stir for 2 hr.
After filtration, the solution is added with cold isopropyl ether, and the peptide is precipitated collected by centrifugation (3 min at 3000 rpm).
The precipitate is washed with cold isopropyl ether for two additional times.
Dry the crude peptide under vacuum for 2 hr.
Dissolve the crude peptide in ACN/H₂O (1:1, 600 mL in total)
The disulfide bond is formed through I₂/MeOH and stir for 20 min, where the completion of the reaction is indicated by LCMS.
Lyophilize the reaction mixture to get the crude peptide.
Purify the crude peptide by prep-HPLC (A: 0.075% TFA in H₂O, B: ACN) to give the compound 8 (55.0 mg).

A mixture of compound 8 (55.0 mg, 1.0 eq) and BCN-NHS (7.20 mg, 1.1 eq) was dissolved in DMF (1 mL), and then DIEA (6.0 eq) was added slowly. The mixture was stirred at 20° C. for 8 hr. LCMS showed the reaction was complete. The mixture was then directly purified by prep-HPLC (TFA condition), and compound 9 (19.0 mg, 32.0% yield) was obtained as a white solid.

A mixture of compound 9 (19.0 mg, 1.0 eq) and compound 6 (13.5 mg, 1.0 eq) was dissolved in DMF (1.0 mL), and the reaction was stirred at 20° C. for 8 hr. LCMS showed the reaction was complete, and then the mixture was directly purified by prep-HPLC. Fractions with desired m/z (e.g., 1126.0, 901.2, 751.1, etc.) were combined and lyophilized to produce I-6 (12.7 mg, 39.2% yield, 92.4% purity) as a white solid. Purification conditions:

Separation condition

| | |
|---|---|
| Sample Preparation | Dissolve in DMF/H₂O |
| Instrument | Gilson GX-215 |
| Mobile Phase | A: H₂O (0.075% TFA in H₂O) |
| | B: CH₃CN |
| Gradient | 30-60%-60 min. Retention time: 54 min |
| Column | Luna C18, 25*200 mm, 10 um, 110A + |
| | Gemin C18, 150*30 mm, 5 um, 110A |
| Flow Rate | 20 mL/min |
| Wavelength | 220/254 nm |
| Oven Tem. | Room temperature |

Exemplary Preparation of I-7.

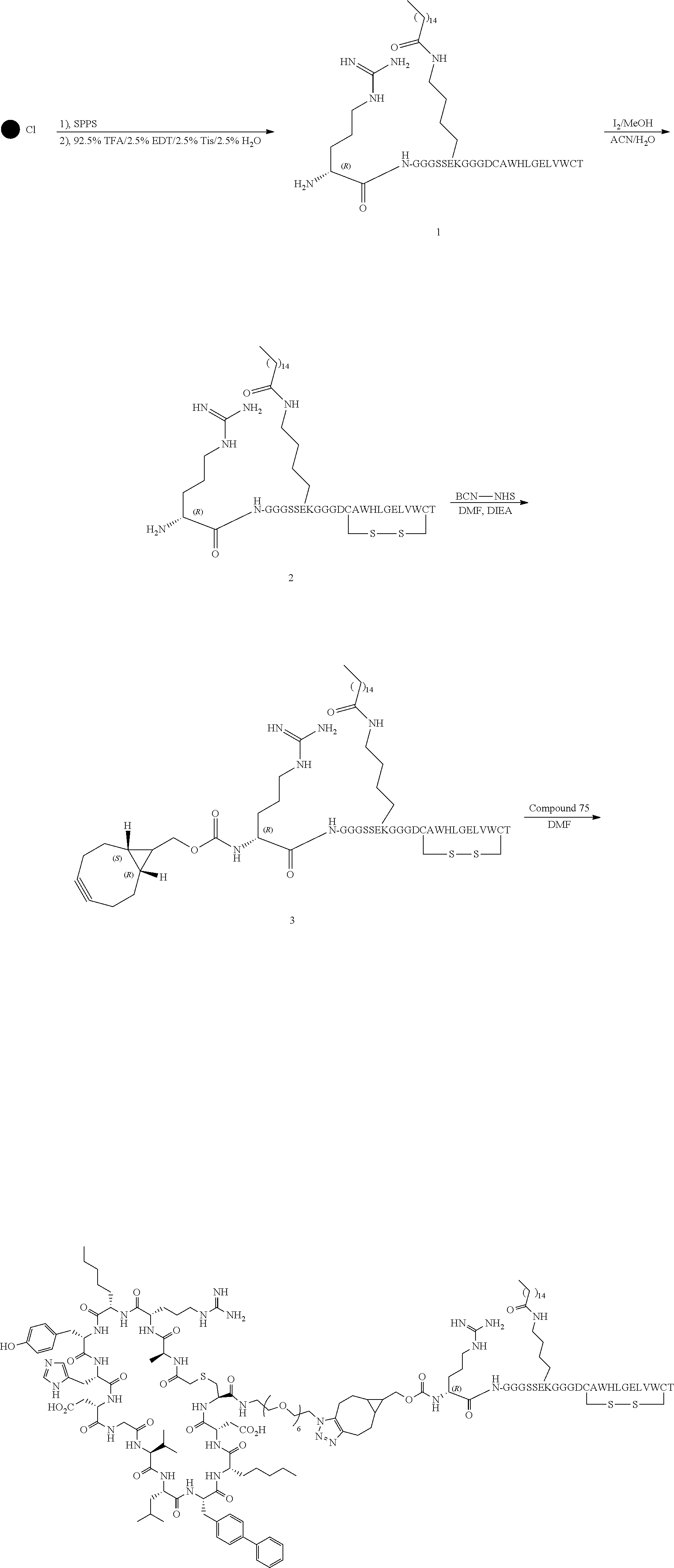

Exemplary Peptide Synthesis. Peptide were synthesized using standard Fmoc chemistry, for example:

1) Add DCM to the vessel containing CTC Resin (0.500 mmol, 500 mg, 1.00 mmol/g) and Fmoc-Thr(tBu)-OH (159 mg, 0.400 mmol, 0.80 eq) with N₂ bubbling.
2) Add DIEA (4.00 eq) dropwise and mix for 2 hours.

### 3) Add MeOH (0.50 mL) and mix for 30 min.

4) Drain and wash with DMF for 5 times.
5) Add 20% piperidine/DMF and react for 30 min.
6) Drain and wash with DMF for 5 times.
7) Add Fmoc-amino acid solution and mix for 30 sec first, then add activation solution, and the coupling reaction lasts for 1 hr with continuous N₂ bubbling.
8) Repeat step 4 to 7 for next amino acid coupling.

| # | Materials | Coupling reagents |
|---|---|---|
| 1 | Fmoc-Thr(tBu)-OH (0.80 eq) | DIEA (4.00 eq) |
| 2 | Fmoc-Cys(Trt)-OH (3.00 eq) | HBTU(2.85 eq) |
| | | and DIEA (6.00 eq) |
| 3 | Fmoc-Trp(Boc)-OH (3.00 eq) | HBTU(2.85 eq) |
| | | and DIEA (6.00 eq) |
| 4 | Fmoc-Val-OH (3.00 eq) | HBTU(2.85 eq) |
| | | and DIEA (6.00 eq) |
| 5 | Fmoc-Leu-OH (3.00 eq) | HBTU(2.85 eq) |
| | | and DIEA (6.00 eq) |
| 6 | Fmoc-Glu(OtBu)-OH (3.00 eq) | HBTU(2.85 eq) |
| | | and DIEA (6.00 eq) |
| 7 | Fmoc-Gly-OH (3.00 eq) | HBTU(2.85 eq) |
| | | and DIEA (6.00 eq) |
| 8 | Fmoc-Leu-OH (3.00 eq) | HBTU(2.85 eq) |
| | | and DIEA (6.00 eq) |
| 9 | Fmoc-His(Trt)-OH (3.00 eq) | HATU(2.85 eq) |
| | | and DIEA (6.00 eq) |
| 10 | Fmoc-Trp(Boc)-OH (3.00 eq) | HATU(2.85 eq) |
| | | and DIEA (6.00 eq) |
| 11 | Fmoc-Ala-OH (3.00 eq) | HATU(2.85 eq) |
| | | and DIEA (6.00 eq) |
| 12 | Fmoc-Cys(Trt)-OH (3.00 eq) | HATU(2.85 eq) |
| | | and DIEA (6.00 eq) |
| 13 | Fmoc-Asp(OtBu)-OH (3.00 eq) | HATU(2.85 eq) |
| | | and DIEA (6.00 eq) |
| 14 | Fmoc-Gly-Gly-Gly-OH (3.00 eq) | HATU(2.85 eq) |
| | | and DIEA (6.0 eq) |
| 15 | Fmoc-Lys(Dde)-OH (3.00 eq) | HATU(2.85 eq) |
| | | and DIEA (6.0 eq) |
| 16 | Fmoc-Glu(OtBu)-OH (3.00 eq) | HATU(2.85 eq) |
| | | and DIEA (6.00 eq) |
| 17 | Fmoc-Ser(tBu)-Ser[psiMe,- | HATU(2.85 eq) |
| | Mepro]-OH (3.00 eq) | and DIEA (6.0 eq) |
| 18 | Fmoc-Gly-Gly-Gly-OH (3.00 eq) | HATU(2.85 eq) |
| | | and DIEA (6.0 eq) |
| 19 | Boc-D-Arg(Pbf)-OH (3.00 eq) | HATU(2.85 eq) |
| | | and DIEA (6.00 eq) |
| 20 | Palmitic acid (3.00 eq) | HBTU(2.85 eq) |
| | | and DIEA (6.00 eq) |

Synthesis scale: 0.4 mmol
20% piperidine in DMF was used for Fmoc deprotection for 30 min.
The coupling reaction was monitored by ninhydrin test.
3% NH₂NH₂H₂O/DMF was used for De-Dde after cycle 19, and then palmitic acid was coupled to the sidechain of Lys.
After last coupling, the resin was washed with MeOH for 3 times, and then dried under vacuum.

### Peptide Cleavage and Purification:

Add cleavage cocktail (92.5% TFA/2.5% EDT/2.5% H₂O/2.5% TIS) to the flask containing the side chain protected peptide at room temperature and stir for 2 hr.
After filtration, the solution is added with cold isopropyl ether, and the peptide is precipitated collected by centrifugation (3 min at 3000 rpm).
The precipitate is washed with cold isopropyl ether for two additional times.
Dry the crude peptide under vacuum for 2 hr.
Dissolve the crude peptide in ACN/H₂O (1:1, 600 mL in total)
The disulfide bond is formed through I₂/MeOH and stir for 20 min, where the completion of the reaction is indicated by LCMS.
Lyophilize the reaction mixture to get the crude peptide.
Purify the crude peptide by prep-HPLC (A: 0.075% TFA in H₂O, B: ACN) to give the compound 2 (63.0 mg).

A mixture of compound 2 (63.0 mg, 1.0 eq) and BCN-NHS (7.47 mg, 1.1 eq) was dissolved in DMF (1 mL), and then DIEA (6.00 eq) was added slowly. The mixture was stirred at 20° C. for 8 hr. LCMS showed the reaction was complete. The mixture was then directly purified by prep-HPLC (TFA condition), and compound 3 (20.0 mg, 29.8% yield) was obtained as a white solid.

A mixture of compound 3 (20.0 mg, 1.0 eq) and compound 75 (13.9 mg, 1.0 eq) was dissolved in DMF (1.0 mL), and the reaction was stirred at 20° C. for 8 hr. LCMS showed the reaction was complete, and then the mixture was directly purified by prep-HPLC. Fractions with desired m/z (e.g., 1219.3, 975.4, etc.) were combined and lyophilized to produce I-7 (11.7 mg, 34.5% yield, 96.8% purity) as a white solid. Purification conditions:

Separation condition

| | |
|---|---|
| Sample Preparation | Dissolve in DMF/H₂O |
| Instrument | Gilson GX-215 |
| Mobile Phase | A: H₂O (0.075% TFA in H₂O) |
| | B: CH₃CN |
| Gradient | 30-60%-60 min. Retention time: 53 min |
| Column | Luna C18, 25*200 mm, 10 um, 110A + |
| | Gemin C18, 150*30 mm, 5 um, 110A |
| Flow Rate | 20 mL/min |
| Wavelength | 220/254 nm |
| Oven Tem. | Room temperature |

Exemplary Preparation of I-8.

Exemplary Peptide Synthesis. Peptide were synthesized using standard Fmoc chemistry, for example:

1) Add DCM to the vessel containing CTC Resin (0.500 mmol, 500 mg, 1.00 mmol/g) and Fmoc-Thr(tBu)-OH (159 mg, 0.400 mmol, 0.80 eq) with N₂ bubbling.
2) Add DIEA (4.00 eq) dropwise and mix for 2 hours.

### 3) Add MeOH (0.50 mL) and mix for 30 min.

4) Drain and wash with DMF for 5 times.
5) Add 20% piperidine/DMF and react on 30 min.
6) Drain and wash with DMF for 5 times.
7) Add Fmoc-amino acid solution and mix for 30 sec first, then add activation solution, and the coupling reaction lasts for 1 hr with continuous N₂ bubbling.
8) Repeat step 4 to 7 for next amino acid coupling.

| # | Materials | Coupling reagents |
|---|---|---|
| 1 | Fmoc-Thr(tBu)—OH (0.80 eq) | DIEA (4.00 eq) |
| 2 | Fmoc-Cys(Trt)—OH (3.00 eq) | HBTU(2.85 eq) and DIEA (6.00 eq) |
| 3 | Fmoc-Trp(Boc)—OH (3.00 eq) | HBTU(2.85 eq) and DIEA (6.00 eq) |
| 4 | Fmoc-Val—OH (3.00 eq) | HBTU(2.85 eq) and DIEA (6.00 eq) |
| 5 | Fmoc-Leu—OH (3.00 eq) | HBTU(2.85 eq) and DIEA (6.00 eq) |
| 6 | Fmoc-Glu(OtBu)—OH (3.00 eq) | HBTU(2.85 eq) and DIEA (6.00 eq) |
| 7 | Fmoc-Gly—OH (3.00 eq) | HBTU(2.85 eq) and DIEA (6.00 eq) |
| 8 | Fmoc-Leu—OH (3.00 eq) | HBTU(2.85 eq) and DIEA (6.00 eq) |
| 9 | Fmoc-His(Trt)—OH (3.00 eq) | HATU(2.85 eq) and DIEA (6.00 eq) |
| 10 | Fmoc-Trp(Boc)—OH (3.00 eq) | HATU(2.85 eq) and DIEA (6.00 eq) |
| 11 | Fmoc-Ala—OH (3.00 eq) | HATU(2.85 eq) and DIEA (6.00 eq) |
| 12 | Fmoc-Cys(Trt)—OH (3.00 eq) | HATU(2.85 eq) and DIEA (6.00 eq) |
| 13 | Fmoc-Asp(OtBu)—OH (3.00 eq) | HATU(2.85 eq) and DIEA (6.00 eq) |
| 14 | Fmoc-Gly-Gly—OH (3.00 eq) | HATU(2.85 eq) and DIEA (6.0 eq) |
| 15 | Fmoc-D-gamaGlu(OtBu)—OH (3.00 eq) | HATU(2.85 eq) and DIEA (6.0 eq) |
| 16 | Fmoc-D-gamaGlu(OtBu)—OH (3.00 eq) | HATU(2.85 eq) and DIEA (6.00 eq) |
| 17 | Fmoc-D-Glu(OAll)—OH (3.00 eq) | HATU(2.85 eq) and DIEA (6.00 eq) |
| 18 | Fmoc-D-gamaGlu(OtBu)—OH (3.00 eq) | HATU(2.85 eq) and DIEA (6.00 eq) |
| 19 | Fmoc-D-gamaGlu(OtBu)—OH (3.00 eq) | HATU(2.85 eq) and DIEA (6.00 eq) |
| 20 | Fmoc-Gly—OH (3.00 eq) | HATU(2.85 eq) and DIEA (6.00 eq) |
| 21 | Fmoc-piperidine-4-carboxylic acid(3.00 eq) | HATU(2.85 eq) and DIEA (6.00 eq) |
| 22 | Palmitic acid (3.00 eq) | HBTU(2.85 eq) and DIEA (6.00 eq) |

Synthesis scale: 0.4 mmol
20% piperidine in DMF was used for Fmoc deprotection for 30 min.
Pd(PPh₃)₄ and phenylsilane was used for De-OAll.
The coupling reaction was monitored by ninhydrin test.
After last amino acid coupling, N-terminal Fmoc was removed, and the resin was washed with MeOH for 3 times, and then dried under vacuum.

### Peptide Cleavage and Purification:

Add cleavage cocktail (95% TFA/2.5% EDT/2.5% H₂O/2.5% TIS) to the flask containing the side chain protected peptide at room temperature and stir for 2 hr.
The peptide is precipitated with cold isopropyl ether and collected by centrifugation (3 min at 3000 rpm).
The precipitate is washed with cold isopropyl ether for two additional times.
Dry the crude peptide under vacuum for 2 hr.
Dissolve the crude peptide in ACN/H₂O (1:1, 600 mL in total)
Adjust pH to 8 by NaHCO₃ and stir for 16 hr, and the disulfide bond is formed through air oxidation, where the completion of the reaction is indicated by LCMS.
Lyophilize the reaction mixture to get the crude peptide.
Purify the crude peptide by prep-HPLC (A: 0.075% TFA in H₂O, B: ACN) to give the compound 2 (75.0 mg).

A mixture of compound 2 (75.0 mg, 1.0 eq) and BCN-NHS (8.91 mg, 1.1 eq) was dissolved in DMF (2 mL), and then DIEA (6.00 eq) was added slowly. The mixture was stirred at 20° C. for 8 hr. LCMS showed the reaction was complete. The mixture was then directly purified by prep-HPLC (TFA condition), and compound 3 (35.0 mg, 43.8% yield) was obtained as a white solid.

A mixture of compound 3 (35.0 mg, 1.0 eq) and compound 75 (24.4 mg, 1.0 eq) was dissolved in DMF (2.0 mL), and the reaction was stirred at 20° C. for 8 hr. LCMS showed the reaction was complete, and then the mixture was directly purified by prep-HPLC. Fractions with desired m/z (e.g., 1218.3, 974.8, 812.2 etc.) were combined and lyophilized to produce I-8 (29.3 mg, 49.7% yield, 97.6% purity) as a white solid. Purification conditions:

Separation condition

| Sample Preparation | Dissolve in DMF/H₂O |
|---|---|
| Instrument | Gilson GX-215 |
| Mobile Phase | A: H₂O (0.075% TFA in H₂O) |
| | B: CH₃CN |
| Gradient | 30-60%-60 min. Retention time: 55 min |
| Column | Luna C18, 25*200 mm, 10 um, 110A + |
| | Gemin C18, 150*30 mm, 5 um, 110A |
| Flow Rate | 20 mL/min |
| Wavelength | 220/254 nm |
| Oven Tem. | Room temperature |

Exemplary Preparation of I-9.

Exemplary Peptide Synthesis. Peptide were synthesized using standard Fmoc chemistry, for example:

1) Add DCM to the vessel containing CTC Resin (0.300 mmol, 300 mg, 1.00 mmol/g) and Fmoc-Thr(tBu)-OH (95.3 mg, 0.240 mmol, 0.80 eq) with N₂ bubbling.
2) Add DIEA (4.00 eq) dropwise and mix for 2 hr.

### 3) Add MeOH (0.30 mL) and mix for 30 min.

4) Drain and wash with DMF for 5 times.
5) Add 20% piperidine/DMF and react on 30 min.
6) Drain and wash with DMF for 5 times.
7) Add Fmoc-amino acid solution and mix for 30 sec first, then add activation solution, and the coupling reaction lasts for 1 hr with continuous N₂ bubbling.
8) Repeat step 4 to 7 for next amino acid coupling.

| # | Materials | Coupling reagents |
|---|---|---|
| 1 | Fmoc-Thr(tBu)—OH (0.80 eq) | DIEA (4.00 eq) |
| 2 | Fmoc-Cys(Trt)—OH (3.00 eq) | HBTU(2.85 eq) and DIEA (6.00 eq) |
| 3 | Fmoc-Trp(Boc)—OH (3.00 eq) | HBTU(2.85 eq) and DIEA (6.00 eq) |
| 4 | Fmoc-Val—OH (3.00 eq) | HBTU(2.85 eq) and DIEA (6.00 eq) |
| 5 | Fmoc-Leu—OH (3.00 eq) | HBTU(2.85 eq) and DIEA (6.00 eq) |
| 6 | Fmoc-Glu(OtBu)—OH (3.00 eq) | HBTU(2.85 eq) and DIEA (6.00 eq) |
| 7 | Fmoc-Gly—OH (3.00 eq) | HBTU(2.85 eq) and DIEA (6.00 eq) |
| 8 | Fmoc-Leu—OH (3.00 eq) | HBTU(2.85 eq) and DIEA (6.00 eq) |
| 9 | Fmoc-His(Trt)—OH (3.00 eq) | HATU(2.85 eq) and DIEA (6.00 eq) |
| 10 | Fmoc-Trp(Boc)—OH (3.00 eq) | HATU(2.85 eq) and DIEA (6.00 eq) |
| 11 | Fmoc-Ala—OH (3.00 eq) | HATU(2.85 eq) and DIEA (6.00 eq) |
| 12 | Fmoc-Cys(Trt)—OH (3.00 eq) | HATU(2.85 eq) and DIEA (6.00 eq) |
| 13 | Fmoc-Asp(OtBu)—OH (3.00 eq) | HATU(2.85 eq) and DIEA (6.00 eq) |
| 14 | Fmoc-Gly-Gly—OH (3.00 eq) | HATU(2.85 eq) and DIEA (6.0 eq) |
| 15 | Fmoc-D-gamaGlu(OtBu)—OH (3.00 eq) | HATU(2.85 eq) and DIEA (6.0 eq) |
| 16 | Fmoc-D-gamaGlu(OtBu)—OH (3.00 eq) | HATU(2.85 eq) and DIEA (6.00 eq) |
| 17 | Fmoc-D-Glu(OAll)—OH (3.00 eq) | HATU(2.85 eq) and DIEA (6.00 eq) |
| 18 | Fmoc-D-gamaGlu(OtBu)—OH (3.00 eq) | HATU(2.85 eq) and DIEA (6.00 eq) |
| 19 | Fmoc-D-gamaGlu(OtBu)—OH (3.00 eq) | HATU(2.85 eq) and DIEA (6.00 eq) |
| 20 | Fmoc-Gly—OH (3.00 eq) | HATU(2.85 eq) and DIEA (6.00 eq) |
| 21 | Fmoc-piperidine-4-carboxylic acid (3.00 eq) | HATU(2.85 eq) and DIEA (6.00 eq) |
| 22 | tert-butyl 12-aminododecanoate (3.00 eq) | HOAt(3.0 eq) and DIC (3.00 eq) |

Synthesis scale: 0.4 mmol
20% piperidine in DMF was used for Fmoc deprotection for 30 min.
Pd(PPh₃)₄ and phenylsilane was used for removing Ally group on the sidechain of D-Glu.
The coupling reaction was monitored by ninhydrin test.
After last amino acid coupling, N-terminal Fmoc was removed, and the resin was washed with MeOH for 3 times, and then dried under vacuum.

### Peptide Cleavage and Purification:

Add cleavage cocktail (95% TFA/2.5% EDT/2.5% H₂O/2.5% TIS) to the flask containing the side chain protected peptide at room temperature and stir for 2 hr.
The peptide is precipitated with cold isopropyl ether and collected by centrifugation (3 min at 3000 rpm).
The precipitate is washed with cold isopropyl ether for two additional times.
Dry the crude peptide under vacuum for 2 hr.
Dissolve the crude peptide in ACN/H₂O (1:1, 600 mL in total)
Adjust pH to 8 by NaHCO₃ and stir for 16 hr, and the first disulfide bond is formed through air oxidation, where the completion of the reaction is indicated by LCMS.
Lyophilize the reaction mixture to get the crude peptide.
Purify the crude peptide by prep-HPLC (A: 0.075% TFA in H₂O, B: ACN) to give the compound 2 (42.0 mg).

A mixture of compound 2 (42.0 mg, 1.0 eq) and BCN-NHS (5.06 mg, 1.1 eq) was dissolved in DMF (1 mL), and then DIEA (6.00 eq) was added slowly. The mixture was stirred at 20° C. for 8 hr. LCMS showed the reaction was complete. The mixture was then directly purified by prep-HPLC (TFA condition), and compound 3 (13.0 mg, 29.0% yield) was obtained as a white solid.

A mixture of compound 3 (13.0 mg, 1.0 eq) and compound 75 (9.18 mg, 1.0 eq) was dissolved in DMF (1.0 mL), and the reaction was stirred at 20° C. for 8 hr. LCMS showed the reaction was complete, and then the mixture was directly purified by prep-HPLC. Fractions with desired m/z (e.g., 1207.9, 966.8, 805.6, etc.) were combined and lyophilized to produce I-9 (10.9 mg, 49.1% yield, 97.1% purity) as a white solid. Purification conditions:

Separation condition

| Sample Preparation | Dissolve in DMF/H₂O |
|---|---|
| Instrument | Gilson GX-215 |
| Mobile Phase | A: H₂O (0.075% TFA in H₂O) |
| | B: CH₃CN |
| Gradient | 20-50%-60 min. Retention time: 50 min |
| Column | Luna C18, 25*200 mm, 10 um, 110A + |
| | Gemin C18, 150*30 mm, 5 um, 110A |
| Flow Rate | 20 mL/min |
| Wavelength | 220/254 nm |
| Oven Tem. | Room temperature |

Exemplary Preparation of I-10.

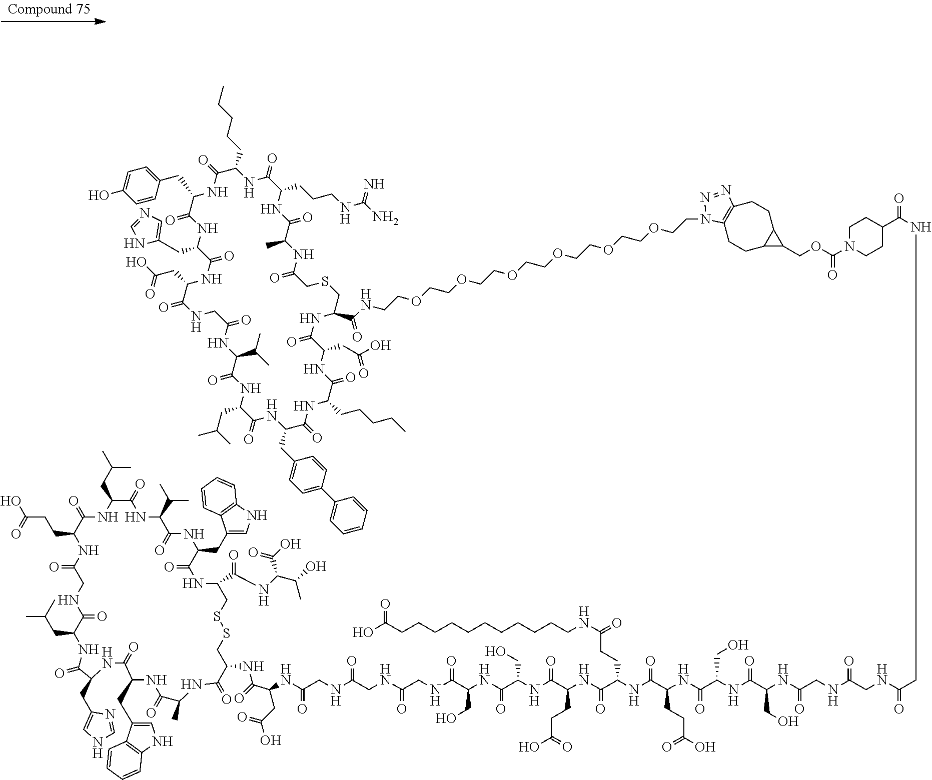

Azide containing compound 75 (1.0 eq) and a corresponding alkyne-containing compound 3 (1.0 eq), prepared using similar technologies described above, were dissolved in DMF (1.0 mL), and the reaction was stirred at 20° C. for 8 hr. LCMS showed the reaction was complete, and then the mixture was directly purified by prep-HPLC. Fractions with desired m/z (e.g., 1019.0, 849.0, etc.) were combined and lyophilized to produce I-10 (7.3 mg, 33.2% yield, 98.9% purity) as a white solid. Purification conditions:

Separation condition

| | |
|---|---|
| Sample Preparation | Dissolve in DMF/H₂O |
| Instrument | Gilson GX-215 |
| Mobile Phase | A: H₂O (0.075% TFA in H₂O) |
| | B: CH₃CN |
| Gradient | 20-50%-60 min. Retention time: 55 min |
| Column | Luna C18, 25*200 mm, 10 um, 110A + |
| | Gemin C18, 150*30 mm, 5 um, 110A |
| Flow Rate | 20 mL/min |
| Wavelength | 220/254 nm |
| Oven Tem. | Room temperature |

Exemplary Preparation of I-11.

Exemplary Peptide Synthesis. Peptide were synthesized using standard Fmoc chemistry, for example:

1) Add DCM (2.00 mL) to the vessel containing CTC Resin (0.50 mmol, 0.45 g, 1.10 mmol/g) and Fmoc-NH-PEG₆-CH₂CH₂COOH (0.30 g, 0.50 mmol, 1.00 eq) with N₂ bubbling.
2) Add DIEA (4.00 eq) dropwise and mix for 2 hr.

### 3) Add MeOH (0.50 mL) and mix for 30 min.

4) Drain and wash with DMF for 5 times.
5) Add 20% piperidine/DMF and react for 30 min.
6) Drain and wash with DMF for 5 times.
7) Add Fmoc-amino acid solution and mix for 30 sec first, then add activation buffer, and the coupling reaction lasts for 1 hr with continuous N₂ bubbling.
8) Repeat step 4 to 7 for next amino acid coupling.

| Step | Materials | Coupling reagents |
|---|---|---|
| 1 | Fmoc-NH-PEG₆—CH₂CH₂COOH (1.00 eq) | DIEA (4.00 eq) |
| 2 | Fmoc-Arg(Pbf)—OH (3.00 eq) | HBTU (2.85 eq) and DIEA (6.00 eq) |
| 3 | Fmoc-Arg(Pbf)—OH (3.00 eq) | HBTU (2.85 eq) and DIEA (6.00 eq) |
| 4 | Fmoc-Inp—OH (3.00 eq) | HBTU (2.85 eq) and DIEA (6.00 eq) |
| 5 | Boc₂O (3.00 eq) | DIEA (6.00 eq) |

Synthesis scale: 0.5.50 mol
20% piperidine in DMF was used for Fmoc deprotection for 30 min. The coupling reaction was monitored by ninhydrin test, and the resin was washed with DMF for 5 times.
After last amino acid coupling, the resin was washed with MeOH for 3 times, and then dried under vacuum.

### Peptide Cleavage:

Add cleavage solution (20% HFIP/DCM, 50 mL) to the flask containing the side chain protected peptide at room temperature. The cleavage was carried out twice (30 min each), with continuous N₂ bubbling.
After filtered, the filtrate was concentrated under reduced pressure and the residue was dried in lyophilizer to give compound 1 (0.50 g, crude) as a white solid.

A solution of compound 1 (50.0 mg, 36.0 μmol), N-[3-(3-aminopropylcarbamoyl)-4-methoxy-phenyl] imidazo[2,1-b]thiazole-6-carboxamide (17.0 mg, 36.3 μmol, TFA salt), EDCI (69.1 mg, 362 μmol) in pyridine (0.50 mL) was stirred at 25° C. for 16 hr. Then pyridine was removed under reduced pressure, resulting a residue which was then added with TFA (1.00 mL) and stirred at 25° C. for 1 hr to remove Boc protecting group. The solvent was removed under reduced pressure, and the residue was purified by prep-HPLC (acidic condition, TFA) to get compound 2 (TFA salt, 35.0 mg, 30.9 μmol, 85.4% yield) as a white solid.

A solution of compound 2 (35.0 mg, 28.1 μmol, TFA salt), [(1R,8S)-9-bicyclo[6.1.0]non-4-ynyl]methyl (2,5-dioxopyrrolidin-1-yl) carbonate (12.2 mg, 42.1 μmol), DIEA (36.3 mg, 281 μmol, 49 L) in DMF (0.20 mL) was stirred at 25° C. for 3 hr. The solution was then purified by prep-HPLC (acidic condition, TFA) to get compound 3 (30.0 mg, 22.9 μmol, 81.6% yield) as a white solid.

A solution of compound 75 (39.7 mg, 19.8 μmol), compound 3 (26.0 mg, 19.8 μmol), DIEA (10.2 mg, 79.4 μmol, 14 μL) in DMF (0.70 mL) was stirred at 25° C. for 3 hr. When click reaction was complete (indicated by LCMS, observed MS: m/z 1102.3, 826.8, 661.6, etc.), the solution was directly purified by prep-HPLC (acid condition, TFA), resulting in compound I-11 (29.0 mg, 8.33 μmol, 41.9% yield, 98.4% purity) as a white solid after lyophilization. Purification conditions:

Purification condition

| | |
|---|---|
| Sample Preparation | Dissolve in MeCN/H₂O |
| Instrument | Gilson GX-215 |
| Mobile Phase | A: H₂O (0.075% TFA in H₂O) |
| | B: MeCN |
| Gradient | 12-42%-60 min. Retention time: 44 min |
| Column | Luna25*200 mm, C18 10 um, 110A + |
| | Gemin150*30 mm, C18 5 um, 110A |
| Flow Rate | 20 mL/min |
| Wavelength | 220/254 nm |
| Oven Tem. | Room temperature |

Exemplary Preparation of I-12.

Exemplary Peptide Synthesis. Peptide were synthesized using standard Fmoc chemistry, for example:

1) Add DCM (2.00 mL) to the vessel containing CTC Resin (0.50 mmol, 0.45 g, 1.1 mmol/g) and Fmoc-NH-PEG₆-CH₂CH₂COOH (0.30 g, 0.50 mmol, 1.00 eq) with N₂ bubbling.
2) Add DIEA (4.00 eq) dropwise and mix for 2 hr.

### 3) Add MeOH (0.50 mL) and mix for 30 min.

4) Drain and wash with DMF for 5 times.
5) Add 20% piperidine/DMF and react for 30 min.
6) Drain and wash with DMF for 5 times.
7) Add Fmoc-amino acid solution and mix for 30 sec first, then add activation buffer, and the coupling reaction lasts for 1 hr with continuous N₂ bubbling.
8) Repeat step 4 to 7 for next amino acid coupling.

| Step | Materials | Coupling reagents |
|---|---|---|
| 1 | Fmoc-NH-PEG₆—CH₂CH₂COOH (1.00 eq) | DIEA (4.00 eq) |
| 2 | Fmoc-Arg(Pbf)—OH (3.00 eq) | HBTU (2.85 eq) and DIEA (6.00 eq) |
| 3 | Fmoc-Arg(Pbf)—OH (3.00 eq) | HBTU (2.85 eq) and DIEA (6.00 eq) |
| 4 | Fmoc-Inp—OH (3.00 eq) | HBTU (2.85 eq) and DIEA (6.00 eq) |
| 5 | Boc₂O (3.00 eq) | DIEA (6.00 eq) |

Synthesis scale: 0.50 mmol.
20% piperidine in DMF was used for Fmoc deprotection for 30 min. The coupling reaction was monitored by ninhydrin test, and the resin was washed with DMF for 5 times.
After last amino acid coupling, the resin was washed with MeOH for 3 times, and then dried under vacuum.

### Peptide Cleavage:

Add cleavage solution (20% HFIP/DCM, 50 mL) to the flask containing the side chain protected peptide at room temperature. The cleavage was carried out twice (30 min each), with continuous N₂ bubbling.
After filtered, the filtrate was concentrated under reduced pressure and the residue was dried in lyophilizer to give compound 1 (0.50 g, crude) as a white solid.

A solution of compound 1 (50.0 mg, 36.1 μmol), 1-(3-aminopropyl)-N-[5-(2-furyl)-1,3,4-thiadiazol-2-yl]-2-oxo-3H-benzimidazole-5-carboxamide (18.0 mg, 36.1 μmol, TFA salt), EDCI (69.3 mg, 362 μmol) in pyridine (0.50 mL) was stirred at 25° C. for 16 hr. Then pyridine was removed under reduced pressure. The residue was added TFA (1 mL) and stirred at 25° C. for 1 hr. The solvent was removed under reduced pressure. The residue was purified by prep-HPLC (acidic condition, TFA) to get compound 2 (TFA salt, 25.0 mg, 21.8 μmol, 60.4% yield) as a white solid.

A solution of compound 2 (25.0 mg, 19.8 μmol, TFA salt), [(1R,8S)-9-bicyclo[6.1.0]non-4-ynyl]methyl (2,5-dioxopyrrolidin-1-yl) carbonate (5.8 mg, 19.8 μmol), DIEA (12.8 mg, 99.4 μmol, 18 L) in DMF (0.2 mL) was stirred at 25° C. for 3 hr. The solution was purified by prep-HPLC (acidic condition, TFA) to get compound 3 (15 mg, 11.3 μmol, 57.1% yield) as a white solid.

A solution of compound 75 (22.7 mg, 11 μmol), compound 3 (15.0 mg, 11 μmol) in DMF (0.7 mL) was stirred at 25° C. for 3 hr. The solution was purified by prep-HPLC (acid condition, TFA) to get I-12 (25.9 mg, 7.65 μmol, 67.3% yield, 98.5% purity) as a white solid. LCMS observed MS: m/z 1105.8, 829.5, 663.7, etc. Purification conditions:

Purification condition

| | |
|---|---|
| Sample Preparation | Dissolve in MeCN/H₂O |
| Instrument | Gilson GX-215 |
| Mobile Phase | A: H₂O (0.075% TFA in H₂O) |
| | B: MeCN |
| Gradient | 12-42%-60 min. Retention time: 47 min |
| Column | Luna25*200 mm, C18 10 um, 110A + Gemin150*30 mm, C18 5 um, 110A |
| Flow Rate | 20 mL/min |
| Wavelength | 220/254 nm |
| Oven Tem. | Room temperature |

Exemplary Preparation of I-13.

Exemplary Peptide Synthesis. Peptide were synthesized using standard Fmoc chemistry, for example:

1) Add DCM to the vessel containing CTC Resin (0.300 mmol, 300 mg, 1.00 mmol/g) and Fmoc-NH-PEG-CH₂CH₂COOH (0.30 g, 0.50 mmol, 1.00 eq) with N₂ bubbling.
2. Add DIEA (4.00 eq) dropwise and mix for 2 hr.

### 3. Add MeOH (0.50 mL) and mix for 30 min.

4. Drain and wash with DMF for 5 times.
5. Add 20% piperidine/DMF and react for 30 min.
6. Drain and wash with DMF for 5 times.
7. Add Fmoc-amino acid solution and mix for 30 sec first, then add activation buffer, and the coupling reaction lasts for 1 hr with continuous N₂ bubbling.
8. Repeat step 4 to 7 for next amino acid coupling.

| Step | Materials | Coupling reagents |
|---|---|---|
| 1 | Fmoc-NH-PEG₆—CH₂CH₂COOH (1.00 eq) | DIEA (4.00 eq) |
| 2 | Fmoc-Arg(Pbf)—OH (3.00 eq) | HBTU (2.85 eq) and DIEA (6.00 eq) |
| 3 | Fmoc-Arg(Pbf)—OH (3.00 eq) | HBTU (2.85 eq) and DIEA (6.00 eq) |
| 4 | Fmoc-Inp—OH (3.00 eq) | HBTU (2.85 eq) and DIEA (6.00 eq) |
| 5 | Boc₂O (3.00 eq) | DIEA (6.00 eq) |

Synthesis scale: 0.50 mmol
20% piperidine in DMF was used for Fmoc deprotection for 30 min. The coupling reaction was monitored by ninhydrin test, and the resin was washed with DMF for 5 times.
After last amino acid coupling, the resin was washed with MeOH for 3 times, and then dried under vacuum.

### Peptide Cleavage:

Add cleavage solution (20% HFIP/DCM, 50 mL) to the flask containing the side chain protected peptide at room temperature. The cleavage was carried out twice (30 min each), with continuous N₂ bubbling.
After filtered, the filtrate was concentrated under reduced pressure and the residue was dried in lyophilizer to give compound 1 (0.50 g, crude) as a white solid.

A solution of compound 1 (50.0 mg, 36.1 μmol), 1-(3-aminopropyl)-2-methyl-N-[4-(2-pyridyl)thiazol-2-yl] benzimidazole-5-carboxamide (14.2 mg, 36.1 μmol, TFA salt), EDCI (69.3 mg, 362 mol) in pyridine (0.50 mL) was stirred at 25° C. for 16 hr. Then pyridine was removed under reduced pressure. The residue was added with TFA (1.00 mL) and stirred at 25° C. for 1 hr to remove Boc group. After deprotection, the solvent was removed under reduced pressure, and the residue was purified by prep-HPLC (acidic condition, TFA) to get compound 2 (29.0 mg, 16.5 μmol, 45.6% yield) as a white solid.

A solution of compound 2 (29.0 mg, 22.9 μmol, TFA salt), [(1R,8S)-9-bicyclo[6.1.0]non-4-ynyl]methyl (2,5-dioxopyrrolidin-1-yl) carbonate (6.7 mg, 22.9 μmol), DIEA (14.8 mg, 114 μmol, 14.8 L) in DMF (0.20 mL) was stirred at 25° C. for 3 hr. The solution was purified by prep-HPLC (acidic condition, TFA) to get I-13 (TFA salt, 20.0 mg, 15.0 μmol, 65.7% yield) as a white solid.

A solution of compound 75 (30.2 mg, 15.1 μmol), compound 3 (20.0 mg, 15.1 μmol) in DMF (0.70 mL) was stirred at 25° C. for 3 hr. The solution was directly purified by prep-HPLC (acidic condition, TFA) to get I-13 (27.2 mg, 8.06 μmol, 53.1% yield, 99.0% purity) as a white solid after lyophilization. LCMS observed MS: 1108.6, 831.4, 665.4, etc. Purification conditions:

Purification condition

| | |
|---|---|
| Sample Preparation | Dissolve in MeCN/H₂O |
| Instrument | Gilson GX-215 |
| Mobile Phase | A: H₂O (0.075% TFA in H₂O) |
| | B: MeCN |
| Gradient | 12-42%-60 min. Retention time: 39 min |
| Column | Luna25*200 mm, C18 10 um, 110A + Gemin 150*30 mm, C18 5 um, 110A |
| Flow Rate | 20 mL/min |
| Wavelength | 220/254 nm |
| Oven Tem. | Room temperature |

Exemplary Preparation of I-14.

A mixture of compound 1 (1.00 g, 2.4 mmol), Fmoc-NH-PEG₃-CH₂CH₂N₃ (1.10 g, 2.4 mmol), EDCI (688 mg, 3.6 mmol), HOBt (486 mg, 3.6 mmol) was dissolved in DCM (30.0 mL), and the reaction was stirred at 15° C. for 16 hr. The solution was then diluted with DCM (100 mL), washed with 1 M HCl (30 mL), H₂O (30 mL), brine (30 mL), dried over anhydrous Na₂SO₄, concentrated under reduced pressure to get compound 2 (3.00 g, crude) as colorless oil.

Compound 2 (3.00 g, crude) was treated with TFA/H₂O (95/5, 20 mL in total) for 1 hr at 15° C. The solvent was then removed under reduced pressure. The solution was then diluted with DCM (100 mL), washed with 1 M HCl (30 mL), H₂O (30 mL), brine (30 mL), dried over anhydrous Na₂SO₄, concentrated under reduced pressure to get the crude. The crude (2.6 g, 3.67 mmol, 1.0 eq), was first dissolved in DCM, and then DIEA (1.92 mL, 3 eq) was added and mixed well, finally a solution of Boc₂O (1.20 g, 5.51 mmol, 1.27 mL, 1.5 eq, in 20 mL of DCM) was added dropwise at 25° C. This reaction was stirred at 25° C. for 3 hr, and then purified by flash C18 chromatography (ISCO®; 120 g SepaFlash® C18 Flash Column, Eluent of 0-100% MeCN/H₂O @ 75 mL/min) to get compound 3 (1.46 g, 2.24 mmol, 49.2% yield) as a white solid.

Exemplary Peptide Synthesis. Peptide were synthesized using standard Fmoc chemistry, for example:

1) Add DCM to the vessel containing CTC Resin (0.200 mmol, 200 mg, 1.00 mmol/g) and Fmoc-Thr(tBu)-OH (63.5 mg, 0.16 mmol, 0.80 eq) with N₂ bubbling.
2) Add DIEA (4.00 eq) dropwise and mix for 2 hours.

### 3) Add MeOH (0.20 mL) and mix for 30 min.

4) Drain and wash with DMF for 5 times.
5) Add 20% piperidine/DMF and react for 30 min.
6) Drain and wash with DMF for 5 times.
7) Add Fmoc-amino acid solution and mix for 30 sec first, then add activation solution, and the coupling reaction lasts for 1 hr with continuous N₂ bubbling.
8) Repeat step 4 to 7 for next amino acid coupling.

| # | Materials | Coupling reagents |
|---|---|---|
| 1 | Fmoc-Thr(tBu)—OH (0.80 eq) | DIEA (4.00 eq) |
| 2 | Fmoc-Cys(Trt)—OH (3.00 eq) | HBTU(2.85 eq) and DIEA (6.00 eq) |
| 3 | Fmoc-Trp(Boc)—OH (3.00 eq) | HBTU(2.85 eq) and DIEA (6.00 eq) |
| 4 | Fmoc-Val—OH (3.00 eq) | HBTU(2.85 eq) and DIEA (6.00 eq) |
| 5 | Fmoc-Leu—OH (3.00 eq) | HBTU(2.85 eq) and DIEA (6.00 eq) |
| 6 | Fmoc-Glu(OtBu)—OH (3.00 eq) | HBTU(2.85 eq) and DIEA (6.00 eq) |
| 7 | Fmoc-Gly—OH (3.00 eq) | HBTU(2.85 eq) and DIEA (6.00 eq) |
| 8 | Fmoc-Leu—OH (3.00 eq) | HBTU(2.85 eq) and DIEA (6.00 eq) |
| 9 | Fmoc-His(Trt)—OH (3.00 eq) | HATU(2.85 eq) and DIEA (6.00 eq) |
| 10 | Fmoc-Trp(Boc)—OH (3.00 eq) | HATU(2.85 eq) and DIEA (6.00 eq) |
| 11 | Fmoc-Ala—OH (3.00 eq) | HATU(2.85 eq) and DIEA (6.00 eq) |
| 12 | Fmoc-Cys(Trt)—OH (3.00 eq) | HATU(2.85 eq) and DIEA (6.00 eq) |
| 13 | Fmoc-Asp(OtBu)—OH (3.00 eq) | HATU(2.85 eq) and DIEA (6.00 eq) |
| 14 | Fmoc-PEG6-CH₂CH₂-COOH (2.00 eq) | HATU(1.9 eq) and DIEA (4.0 eq) |
| 15 | 2-methylcycloprop-2- enecarboxylic acid (2.00 eq) | HATU(1.9 eq) and DIEA (4.0 eq) |

Synthesis scale: 0.16 mmol
20% piperidine in DMF was used for Fmoc deprotection for 30 min.
The coupling reaction was monitored by ninhydrin test.
After last coupling, the resin was washed with MeOH for 3 times, and then dried under vacuum.

### Peptide Cleavage and Purification:

Add cleavage cocktail (92.5% TFA/2.5% EDT/2.5% H₂O/2.5% TIS) to the flask containing the side chain protected peptide at room temperature and stir for 1.5 hr.
The peptide is precipitated with cold isopropyl ether and collected by centrifugation (3 min at 3000 rpm).
The precipitate is washed with cold isopropyl ether for two additional times.
Dry the crude peptide under vacuum for 2 hr.
Dissolve the crude peptide in ACN/H₂O (1:1, 100 mL in total)
Adjust pH to 8 by NaHCO₃ and stir for 8 hr, and the disulfide bond is formed through air oxidation, where the completion of the reaction is indicated by LCMS.
Lyophilize the reaction mixture to get the crude peptide.
Purify the crude peptide by prep-HPLC (A: 0.075% TFA in H₂O, B: ACN) to give the compound 5 (30.0 mg).

Another example:

1) Add DCM to the vessel containing CTC Resin (0.500 mmol, 500 mg, 1.00 mmol/g) and Fmoc-Thr(tBu)-OH (130 mg, 0.40 mmol, 0.80 eq) with N₂ bubbling.
2) Add DIEA (4.00 eq) dropwise and mix for 2 hours.

### 3) Add MeOH (0.50 mL) and mix for 30 min.

4) Drain and wash with DMF for 5 times.
5) Add 20% piperidine/DMF and react for 30 min.
6) Drain and wash with DMF for 5 times.
7) Add Fmoc-amino acid solution and mix for 30 sec first, then add activation solution, and the coupling reaction lasts for 1 hr with continuous N₂ bubbling.
8) Repeat step 4 to 7 for next amino acid coupling.

| Step | Materials | Coupling reagents |
|---|---|---|
| 1 | Fmoc-Abu—OH (0.8 eq) | DIEA (4.0 eq) |
| 2 | Fmoc-Heptanoic acid (2.0 eq) | HATU (1.9 eq) and DIEA (4.0 eq) |
| 3 | Fmoc-Bip—OH (2.0 eq) | HATU (1.9 eq) and DIEA (4.0 eq) |
| 4 | Fmoc-Leu—OH (3.0 eq) | HBTU (2.85 eq) and DIEA (6.0 eq) |
| 5 | Fmoc-Val—OH (3.0 eq) | HBTU (2.85 eq) and DIEA (6.0 eq) |
| 6 | Fmoc-Gly—OH (3.0 eq) | HBTU (2.85 eq) and DIEA (6.0 eq) |
| 7 | Fmoc-Asp(tBu)—OH (3.0 eq) | HBTU (2.85 eq) and DIEA (6.0 eq) |
| 8 | Fmoc-His(Trt)—OH (3.0 eq) | HBTU (2.85 eq) and DIEA (6.0 eq) |
| 9 | Fmoc-Tyr(tBu)—OH (3.0 eq) | HBTU (2.85 eq) and DIEA (6.0 eq) |
| 10 | Fmoc-Heptanoic acid (2.0 eq) | HATU (1.9 eq) and DIEA (4.0 eq) |
| 11 | Fmoc-Arg(Pbf)—OH (3.0 eq) | HBTU (2.85 eq) and DIEA (6.0 eq) |
| 12 | Fmoc-Ala—OH (3.0 eq) | HBTU (2.85 eq) and DIEA (6.0 eq) |
| 13 | Compound 3 (2.0 eq) | HATU (1.9 eq) and DIEA (4.0 eq) |

Synthesis scale: 0.4 mmol.
20% piperidine in DMF was used for Fmoc deprotection for 30 min.
The coupling reaction was monitored by ninhydrin test.
After last coupling, the resin was washed with MeOH for 3 times, and then dried under vacuum.

### Peptide Cleavage and Purification:

Add cleavage solution (20% HFIP/DCM, 20 mL) to the flask containing the side chain protected peptide at room temperature. The cleavage was carried out twice (30 min each), with continuous N₂ bubbling.
After filtered, the filtrate was concentrated under reduced pressure and the residue was dried in lyophilizer to give compound 6 (1.0 g, crude) as a white solid.
A solution of compound 6 (1.0 g, 1.0 eq), HOBt (101 mg, 2.0 eq), TBTU (240 mg, 2.0 eq), DIEA (0.85 mL, 4.0 eq) in DMF (400 mL) was stirred at 15° C. for 1 hr. When cyclization was complete, the solution was then diluted with EA (1.5 L), washed with 1 M HCl (600 mL), brine (400 mL×4), dried over anhydrous Na₂SO₄, concentrated under reduced pressure to get the crude (1.1 g, crude) as colorless oil. Deprotection of crude cyclic peptide was carried out by treating cyclized peptide (1.1 g, crude) with TFA/H₂O (97.5/2.5, 20 mL in total) by continuous stirring for 1.5 hr at 15° C. The solution was triturated with cold isopropyl ether (250 mL) and the precipitate was collected by centrifugation (3 min at 3000 rpm). The precipitate (deprotected peptide) was washed twice with isopropyl ether (50 mL each), following by drying under vacuum for 2 hr. The residue was purified by prep-HPLC (acidic condition, TFA) to give the compound 7 (150 mg).

A mixture of compound 7 (150 mg, 1.0 eq) and 2,5-dioxopyrrolidin-1-yl 2-(4-(6-methyl-1,2,4,5-tetrazin-3-yl)phenyl) acetate (27.8 mg, 1.1 eq) was dissolved in DMF (4.0 mL), and then DIEA (6.0 eq) was added slowly. The mixture was stirred at 20° C. for 8 hr. LCMS showed the reaction was complete. The mixture was then directly purified by prep-HPLC (TFA condition), and compound 8 (48 mg, 28.9% yield) was obtained as a white solid.

A mixture of compound 8 (11.0 mg, 1.0 eq) and compound 5 (9.94 mg, 1.0 eq) was dissolved in DMF/H₂O (4:1 1.5 mL), and the reaction was stirred at 20° C. for 8 hr. LCMS showed the reaction was complete, and then the mixture was directly purified by prep-HPLC. Fractions with desired m/z (e.g., 1358.2, 1018.7, 815.4, etc.) were combined and lyophilized to produce I-14 (2.2 mg, 10.5% yield, 76.4% purity) as a white solid. Purification conditions:

Separation condition

| | |
|---|---|
| Sample Preparation | Dissolve in DMF/H₂O |
| Instrument | Gilson GX-215 |
| Mobile Phase | A: H₂O (0.075% TFA in H₂O) |
| | B: CH₃CN |
| Gradient | 25-55%-60 min. Retention time: 49 min |
| Column | Luna C18, 25*200 mm, 10 um, 110A + Gemin C18, 150*30 mm, 5 um, |
| | 110A |
| Flow Rate | 20 mL/min |
| Wavelength | 220/254 nm |
| Oven Tem. | Room temperature |

Exemplary Preparation of I-15.

Exemplary Peptide Synthesis. Peptide were synthesized using standard Fmoc chemistry, for example:

1) Add DMF to the vessel containing Rink amide MBHA Resin (1.00 mmol, 2.27 g, sub: 0.440 mmol/g) and swell for 2 hours.
2) Drain and wash with DMF for 3 times.
3) Add 20% piperidine/DMF and mix for 30 min.
4) Drain and wash with DMF for 3 times.
5) Add Fmoc-amino acid solution and mix for 30 sec, then add coupling regents, and the coupling reaction lasts for 1 hr with continuous N₂ bubbling.
6) Repeat step 2 to 5 for next amino acid coupling.

| # | Materials | Coupling reagents |
|---|---|---|
| 1 | Fmoc-Cys(Trt)—OH (3.00 eq) | HBTU(2.85 eq) and DIEA (6.000 eq) |
| 2 | Fmoc-Thr(tBu)—OH (3.00 eq) | HBTU(2.85 eq) and DIEA (6.000 eq) |
| 3 | Fmoc-Cys(Acm)—OH (3.00 eq) | HBTU(2.85 eq) and DIEA (6.000 eq) |
| 4 | Fmoc-Trp(Boc)—OH (3.00 eq) | HBTU(2.85 eq) and DIEA (6.00 eq) |
| 5 | Fmoc-Val—OH (3.00 eq) | HBTU(2.85 eq) and DIEA (6.00 eq) |
| 6 | Fmoc-Leu—OH (3.00 eq) | HBTU(2.85 eq) and DIEA (6.00 eq) |
| 7 | Fmoc-Glu(OtBu)—OH (3.00 eq) | HBTU(2.85 eq) and DIEA (6.00 eq) |
| 8 | Fmoc-Gly—OH (3.00 eq) | HBTU(2.85 eq) and DIEA (6.00 eq) |
| 9 | Fmoc-Leu—OH (3.00 eq) | HBTU(2.85 eq) and DIEA (6.00 eq) |
| 10 | Fmoc-His(Trt)—OH (3.00 eq) | HATU(2.85 eq) and DIEA (6.00 eq) |
| 11 | Fmoc-Trp(Boc)—OH (3.00 eq) | HATU(2.85 eq) and DIEA (6.00 eq) |
| 12 | Fmoc-Ala—OH (3.00 eq) | HATU(2.85 eq) and DIEA (6.00 eq) |
| 13 | Fmoc-Cys(Acm)—OH (3.00 eq) | HATU(2.85 eq) and DIEA (6.00 eq) |
| 14 | Fmoc-Asp(OtBu)—OH (3.00 eq) | HATU(2.85 eq) and DIEA (6.00 eq) |
| 15 | Fmoc-Cys(Trt)—OH (3.00 eq) | HATU(2.85 eq) and DIEA (6.00 eq) |
| 16 | Fmoc-Ahx—OH (3.00 eq) | HATU(2.85 eq) and DIEA (6.00 eq) |
| 17 | Fmoc-Gly-Gly—OH (3.00 eq) | HATU(2.85 eq) and DIEA (6.00 eq) |
| 18 | Fmoc-D-gamaGlu(OtBu)—OH (3.00 eq) | HATU(2.85 eq) and DIEA (6.00 eq) |
| 19 | Fmoc-D-gamaGlu(OtBu)—OH (3.00 eq) | HATU(2.85 eq) and DIEA (6.00 eq) |
| 20 | Fmoc-D-Glu(OtBu)—OH (3.00 eq) | HATU(2.85 eq) and DIEA (6.00 eq) |
| 21 | Fmoc-D-gamaGlu(OtBu)—OH (3.00 eq) | HATU(2.85 eq) and DIEA (6.00 eq) |
| 22 | Fmoc-D-gamaGlu(OtBu)—OH (3.00 eq) | HATU(2.85 eq) and DIEA (6.00 eq) |
| 23 | Fmoc-Gly—OH (3.00 eq) | HATU(2.85 eq) and DIEA (6.00 eq) |
| 24 | Fmoc-piperidine-4-carboxylic acid (3.00 eq) | HATU(2.85 eq) and DIEA (6.00 eq) |

Synthesis scale: 1.0 mmol
20% piperidine in DMF was used for Fmoc deprotection for 30 min.
The coupling reaction was monitored by ninhydrin test.
After last amino acid coupling, N-terminal Fmoc was removed, and the resin was washed with MeOH for 3 times, and then dried under vacuum.

### Peptide Cleavage and Purification:

Add cleavage cocktail (95% TFA/2.5% EDT/2.5% H₂O/2.5% TIS) to the flask containing the side chain protected peptide at room temperature and stir for 2 hr.
The peptide is precipitated with cold isopropyl ether and collected by centrifugation (3 min at 3000 rpm).
The precipitate is washed with cold isopropyl ether for two additional times.
Dry the crude peptide under vacuum for 2 hr.
Dissolve the crude peptide in ACN/H₂O (1:1, 600 mL in total)
Adjust pH to 8 by NaHCO₃ and stir for 16 hr, and the first disulfide bond is formed through air oxidation, where the completion of the reaction is indicated by LCMS.

Lyophilize the reaction mixture to get the crude peptide.

Purify the crude peptide by prep-HPLC (A: 0.075% TFA in H₂O, B: ACN) to give the compound 2 (218 mg, 7.46% yield).
Compound 2 (218 mg) was dissolved in ACN/H₂O (50.0 mL), and then 1 M HCl was added to adjust pH to 1, following by dropwise addition of 0.1 M I₂/AcOH till the mixture turned brown. The mixture was then stirred at 20° C. for 10 hr. LCMS showed the reaction was complete. The mixture was purified by prep-HPLC (TFA condition), resulting compound 3 (100 mg, 48.2% yield) obtained as a white solid.

A mixture of compound 3 (100 mg, 1.0 eq) and BCN-NHS (11.5 mg, 1.1 eq) was dissolved in DMF (3 mL), and then DIEA (6.00 eq) was added slowly. The mixture was stirred at 30° C. for 8 hr. LCMS showed the reaction was complete. The mixture was then directly purified by prep-HPLC (TFA condition), and compound 4 (29.0 mg, 27.4% yield) was obtained as a white solid.

A mixture of compound 4 (29.0 mg, 1.0 eq) and compound 75 (19.6 mg, 1.1 eq) was dissolved in DMF (1.0 mL), and the reaction was stirred at 15° C. for 8 hr. LCMS showed the reaction was complete, and then the mixture was directly purified by prep-HPLC. Fractions with desired m/z (e.g., 1238.0, 990.2, etc.) were combined and lyophilized to produce I-15 (30.6 mg, 59.7% yield, 97.6% purity) as a white solid. Purification condition:

Separation condition

| | |
|---|---|
| Sample Preparation | Dissolve in DMF/H₂O |
| Instrument | Gilson GX-215 |
| Mobile Phase | A: H₂O (0.075% TFA in H₂O) |
| | B: CH₃CN |
| Gradient | 20-50%-60 min. Retention time: 54 min |
| Column | Luna C18, 25*200 mm, 10 um, 110A + Gemin C18, 150*30 mm, 5 um, |
| | 110A |
| Flow Rate | 20 mL/min |
| Wavelength | 220/254 nm |
| Oven Tem. | Room temperature |

Exemplary Preparation of I-16.

Exemplary Peptide Synthesis. Peptide were synthesized using standard Fmoc chemistry, for example:

1) Add DMF to the vessel containing Rink amide MBHA Resin (1.00 mmol, 2.27 g, sub: 0.440 mmol/g) and swell for 2 hours.
2) Drain and wash with DMF for 3 times.
3) Add 20% piperidine/DMF and mix for 30 min.
4) Drain and wash with DMF for 3 times.
5) Add Fmoc-amino acid solution and mix for 30 sec, then add coupling regents, and the coupling reaction lasts for 1 hr with continuous N₂ bubbling.
6) Repeat step 2 to 5 for next amino acid coupling.

| # | Materials | Coupling reagents |
|---|---|---|
| 1 | Fmoc-Cys(Trt)-OH (3.00 eq) | HBTU (2.85 eq) and DIEA (6.00 eq) |
| 2 | Fmoc-Thr(tBu)-OH (3.00 eq) | HBTU (2.85 eq) and DIEA (6.00 eq) |
| 3 | Fmoc-Cys(Acm)-OH (3.00 eq) | HBTU (2.85 eq) and DIEA (6.0 eq) |
| 4 | Fmoc-Trp(Boc)-OH (3.00 eq) | HBTU (2.85 eq) and DIEA (6.0 eq) |
| 5 | Fmoc-Val-OH (3.00 eq) | HBTU (2.85 eq) and DIEA (6.0 eq) |
| 6 | Fmoc-Leu-OH (3.00 eq) | HBTU (2.85 eq) and DIEA (6.0 eq) |
| 7 | Fmoc-Glu(OtBu)-OH (3.00 eq) | HBTU (2.85 eq) and DIEA (6.0 eq) |
| 8 | Fmoc-Gly-OH (3.00 eq) | HBTU (2.85 eq) and DIEA (6.0 eq) |
| 9 | Fmoc-Leu-OH (3.00 eq) | HBTU (2.85 eq) and DIEA (6.0 eq) |
| 10 | Fmoc-His(Trt)-OH (3.00 eq) | HATU (2.85 eq) and DIEA (6.0 eq) |
| 11 | Fmoc-Trp(Boc)-OH (3.00 eq) | HATU (2.85 eq) and DIEA (6.0 eq) |
| 12 | Fmoc-Ala-OH (3.00 eq) | HATU (2.85 eq) and DIEA (6.0 eq) |
| 13 | Fmoc-Cys(Acm)-OH (3.00 eq) | HATU (2.85 eq) and DIEA (6.0 eq) |
| 14 | Fmoc-Asp(OtBu)-OH (3.00 eq) | HATU (2.85 eq) and DIEA (6.0 eq) |
| 15 | Fmoc-Cys(Trt)-OH (3.00 eq) | HATU (2.85 eq) and DIEA (6.0 eq) |
| 16 | Fmoc-Gly-Gly-OH (3.00 eq) | HATU (2.85 eq) and DIEA (6.0 eq) |
| 17 | Fmoc-D-gamaGlu(OtBu)-OH (3.00 eq) | HATU (2.85 eq) and DIEA (6.0 eq) |
| 18 | Fmoc-D-gamaGlu(OtBu)-OH (3.00 eq) | HATU (2.85 eq) and DIEA (6.00 eq) |
| 19 | Fmoc-D-Glu(OtBu)-OH (3.00 eq) | HATU (2.85 eq) and DIEA (6.00 eq) |
| 20 | Fmoc-D-gamaGlu(OtBu)-OH (3.00 eq) | HATU (2.85 eq) and DIEA (6.00 eq) |
| 21 | Fmoc-D-gamaGlu(OtBu)-OH (3.00 eq) | HATU (2.85 eq) and DIEA (6.00 eq) |
| 22 | Fmoc-Gly-OH (3.00 eq) | HATU (2.85 eq) and DIEA (6.00 eq) |
| 23 | Fmoc-piperidine-4-carboxylic acid (3.00 eq) | HATU (2.85 eq) and DIEA (6.00 eq) |

Synthesis scale: 1.0 mmol.
20% piperidine in DMF was used for Fmoc deprotection for 30 min.
The coupling reaction was monitored by ninhydrin test.
After last amino acid coupling, N-terminal Fmoc was removed, and the resin was washed with MeOH for 3 times, and then dried under vacuum.

### Peptide Cleavage and Purification:

Add cleavage cocktail (95% TFA/2.5% EDT/2.5% H₂O/2.5% TIS) to the flask containing the side chain protected peptide at room temperature and stir for 2 hr.
The peptide is precipitated with cold isopropyl ether and collected by centrifugation (3 min at 3000 rpm).
The precipitate is washed with cold isopropyl ether for two additional times.
Dry the crude peptide under vacuum for 2 hr.
Dissolve the crude peptide in ACN/H₂O (1:1, 600 mL in total)
Adjust pH to 8 by NaHCO₃ and stir for 16 hr, and the first disulfide bond is formed through air oxidation, where the completion of the reaction is indicated by LCMS.
Lyophilize the reaction mixture to get the crude peptide.
Purify the crude peptide by prep-HPLC (A: 0.075% TFA in H₂O, B: ACN) to give compound 2 (248 mg, 8.84% yield).

Compound 2 (248 mg) was dissolved in ACN/H₂O (50.0 mL), and then 1 M HCl was added to adjust pH to 1, following by dropwise addition of 0.1 M I₂/AcOH till the mixture turned brown. The mixture was then stirred at 20° C. for 10 hr. LCMS showed the reaction was complete. The mixture was purified by prep-HPLC (TFA condition), resulting compound 3 (110 mg, 44.4% yield) obtained as a white solid.

A mixture of compound 3 (110 mg, 1.0 eq) and BCN-NHS (13.2 mg, 1.1 eq) was dissolved in DMF (3 mL), and then DIEA (6.00 eq) was added slowly. The mixture was stirred at 30° C. for 8 hr. LCMS showed the reaction was complete. The mixture was then directly purified by prep-HPLC (TFA condition), and compound 4 (21.0 mg, 17.9% yield) was obtained as a white solid.

A mixture of compound 4 (21.0 mg, 1.0 eq) and compound 75 (14.8 mg, 1.1 eq) was dissolved in DMF (1.0 mL), and the reaction was stirred at 15° C. for 8 hr. LCMS showed the reaction was complete, and then the mixture was directly purified by prep-HPLC. Fractions with desired m/z (e.g., 1209.4, 967.7, etc.) were combined and lyophilized to produce I-16 (20.4 mg, 60.0% yield, 99.4% purity) as a white solid. Purification condition:

Separation condition

| | |
|---|---|
| Sample Preparation | Dissolve in DMF/H₂O |
| Instrument | Gilson GX-215 |
| Mobile Phase | A: H₂O (0.075% TFA in H₂O) |
| | B: CH₃CN |
| Gradient | 20-50%-60 min. Retention time: 55 min |
| Column | Luna C18, 25*200 mm, 10 um, 110A + Gemin C18, 150*30 mm, 5 um, |
| | 110A |
| Flow Rate | 20 mL/min |
| Wavelength | 220/254 nm |
| Oven Tem. | Room temperature |

Exemplary Preparation of I-17.

Exemplary Peptide Synthesis. Peptide were synthesized using standard Fmoc chemistry, for example:
1) Add DCM to the vessel containing CTC Resin (1.00 mmol, 1.00 g, 1.00 mmol/g) and Fmoc-Thr(tBu)-OH (3218 mg, 0.800 mmol, 0.80 eq) with N₂ bubbling.
2) Add DIEA (4.00 eq) dropwise and mix for 2 hours.
3) Add MeOH (1.00 mL) and mix for 30 min.
4) Drain and wash with DMF for 5 times.
5) Add 20% piperidine/DMF and react on 30 min.
6) Drain and wash with DMF for 5 times.
7) Add Fmoc-amino acid solution and mix for 30 sec first, then add activation solution, and the coupling reaction lasts for 1 hr with continuous N₂ bubbling.
8) Repeat step 4 to 7 for next amino acid coupling.

| # | Materials | Coupling reagents |
|---|---|---|
| 1 | Fmoc-Thr(tBu)-OH (0.80 eq) | DIEA (4.00 eq) |
| 2 | Fmoc-Cys(Trt)-OH (3.00 eq) | HBTU (2.85 eq) and DIEA (6.00 eq) |
| 3 | Fmoc-Trp(Boc)-OH (3.00 eq) | HBTU (2.85 eq) and DIEA (6.00 eq) |
| 4 | Fmoc-Val-OH (3.00 eq) | HBTU (2.85 eq) and DIEA (6.00 eq) |
| 5 | Fmoc-Leu-OH (3.00 eq) | HBTU (2.85 eq) and DIEA (6.00 eq) |
| 6 | Fmoc-Glu(OtBu)-OH (3.00 eq) | HBTU (2.85 eq) and DIEA (6.00 eq) |
| 7 | Fmoc-Gly-OH (3.00 eq) | HBTU (2.85 eq) and DIEA (6.00 eq) |
| 8 | Fmoc-Leu-OH (3.00 eq) | HBTU (2.85 eq) and DIEA (6.00 eq) |
| 9 | Fmoc-His(Trt)-OH (3.00 eq) | HATU (2.85 eq) and DIEA (6.00 eq) |
| 10 | Fmoc-Trp(Boc)-OH (3.00 eq) | HATU (2.85 eq) and DIEA (6.00 eq) |
| 11 | Fmoc-Ala-OH (3.00 eq) | HATU (2.85 eq) and DIEA (6.00 eq) |
| 12 | Fmoc-Cys(Trt)-OH (3.00 eq) | HATU (2.85 eq) and DIEA (6.00 eq) |
| 13 | Fmoc-Asp(OtBu)-OH (3.00 eq) | HATU (2.85 eq) and DIEA (6.00 eq) |
| 14 | Fmoc-Gly-Gly-OH (3.00 eq) | HATU (2.85 eq) and DIEA (6.0 eq) |
| 15 | Fmoc-D-gamaGlu(OtBu)-OH (3.00 eq) | HATU (2.85 eq) and DIEA (6.0 eq) |
| 16 | Fmoc-D-gamaGlu(OtBu)-OH (3.00 eq) | HATU (2.85 eq) and DIEA (6.00 eq) |
| 17 | Fmoc-D-Glu(OtBu)-OH (3.00 eq) | HATU (2.85 eq) and DIEA (6.00 eq) |
| 18 | Fmoc-D-gamaGlu(OtBu)-OH (3.00 eq) | HATU (2.85 eq) and DIEA (6.00 eq) |
| 19 | Fmoc-D-gamaGlu(OtBu)-OH (3.00 eq) | HATU (2.85 eq) and DIEA (6.00 eq) |
| 20 | Fmoc-Gly-OH (3.00 eq) | HATU (2.85 eq) and DIEA (6.00 eq) |
| 21 | Fmoc-piperidine-4-carboxylic acid (3.00 eq) | HATU (2.85 eq) and DIEA (6.00 eq) |

Synthesis scale: 0.8 mmol
20% piperidine in DMF was used for Fmoc deprotection for 30 min.
The coupling reaction was monitored by ninhydrin test.
After last amino acid coupling, N-terminal Fmoc was removed, and the resin was washed with MeOH for 3 times, and then dried under vacuum.

### Peptide Cleavage and Purification:

Add cleavage cocktail (95% TFA/2.5% EDT/2.5% H₂O/2.5% TIS) to the flask containing the side chain protected peptide at room temperature and stir for 2 hr.
The peptide is precipitated with cold isopropyl ether and collected by centrifugation (3 min at 3000 rpm).
The precipitate is washed with cold isopropyl ether for two additional times.
Dry the crude peptide under vacuum for 2 hr.
Dissolve the crude peptide in ACN/H₂O (1:1, 800 mL in total)
Adjust pH to 8 by NaHCO₃ and stir for 16 hr, and the disulfide bond is formed through air oxidation, where the completion of the reaction is indicated by LCMS.
Lyophilize the reaction mixture to get the crude peptide.
Purify the crude peptide by prep-HPLC (TFA condition) to give the compound 2 (102 mg).

A mixture of compound 2 (102 mg, 1.0 eq) and BCN-NHS (13.3 mg, 1.1 eq) was dissolved in DMF (2 mL), and then DIEA (6.00 eq) was added slowly. The mixture was stirred at 20° C. for 8 hr. LCMS showed the reaction was complete. The mixture was then directly purified by prep-HPLC (TFA condition), and compound 3 (43.0 mg, 39.4% yield) was obtained as a white solid.

A mixture of compound 3 (43.0 mg, 1.0 eq) and compound 75 (32.6 mg, 1.0 eq) was dissolved in DMF (2.0 mL), and the reaction was stirred at 20° C. for 8 hr. LCMS showed the reaction was complete, and then the mixture was directly purified by prep-HPLC. Fractions with desired m/z (e.g., 1158.8, 927.2, 772.7, etc.) were combined and lyophilized to produce I-17 (41.1 mg, 56.9% yield, 98.4% purity) as a white solid. Purification conditions:

Separation condition

| | |
|---|---|
| Sample Preparation | Dissolve in DMF/H₂O |
| Instrument | Gilson GX-215 |
| Mobile Phase | A: H₂O (0.075% TFA in H₂O) |
| | B: CH₃CN |
| Gradient | 20-50%-60 min. Retention time: 53 min |
| Column | Luna C18, 25*200 mm, 10 um, 110A + Gemin C18, 150*30 mm, 5 um, |
| | 110A |
| Flow Rate | 20 mL/min |
| Wavelength | 220/254 nm |
| Oven Tem. | Room temperature |

Exemplary Preparation of I-18.

Exemplary Peptide Synthesis. Peptide were synthesized using standard Fmoc chemistry, for example:

1) Add DCM to the vessel containing CTC Resin (0.300 mmol, 300 mg, 1.00 mmol/g) and Fmoc-Thr(tBu)-OH (95.3 mg, 0.240 mmol, 0.80 eq) with N₂ bubbling.
2) Add DIEA (4.00 eq) dropwise and mix for 2 hr.

### 3) Add MeOH (0.30 mL) and mix for 30 min.

4) Drain and wash with DMF for 5 times.
5) Add 20% piperidine/DMF and react for 30 min.
6) Drain and wash with DMF for 5 times.
7) Add Fmoc-amino acid solution and mix for 30 sec first, then add activation solution, and the coupling reaction lasts for 1 hr with continuous N₂ bubbling.
8) Repeat step 4 to 7 for next amino acid coupling.

| # | Materials | Coupling reagents |
|---|---|---|
| 1 | Fmoc-Thr(tBu)-OH (0.80 eq) | DIEA (4.00 eq) |
| 2 | Fmoc-Cys(Trt)-OH (3.00 eq) | HBTU (2.85 eq) and DIEA (6.00 eq) |
| 3 | Fmoc-Trp(Boc)-OH (3.00 eq) | HBTU (2.85 eq) and DIEA (6.00 eq) |
| 4 | Fmoc-Val-OH (3.00 eq) | HBTU (2.85 eq) and DIEA (6.00 eq) |
| 5 | Fmoc-Leu-OH (3.00 eq) | HBTU (2.85 eq) and DIEA (6.00 eq) |
| 6 | Fmoc-Glu(OtBu)-OH (3.00 eq) | HBTU (2.85 eq) and DIEA (6.00 eq) |
| 7 | Fmoc-Gly-OH (3.00 eq) | HBTU (2.85 eq) and DIEA (6.00 eq) |
| 8 | Fmoc-Leu-OH (3.00 eq) | HBTU (2.85 eq) and DIEA (6.00 eq) |
| 9 | Fmoc-His(Trt)-OH (3.00 eq) | HATU (2.85 eq) and DIEA (6.00 eq) |
| 10 | Fmoc-Trp(Boc)-OH (3.00 eq) | HATU (2.85 eq) and DIEA (6.00 eq) |
| 11 | Fmoc-Ala-OH (3.00 eq) | HATU (2.85 eq) and DIEA (6.00 eq) |
| 12 | Fmoc-Cys(Trt)-OH (3.00 eq) | HATU (2.85 eq) and DIEA (6.00 eq) |
| 13 | Fmoc-Asp(OtBu)-OH (3.00 eq) | HATU (2.85 eq) and DIEA (6.00 eq) |
| 14 | Fmoc-PEG6-CH₂CH₂-COOH (2.00 eq) | HATU (1.9 eq) and DIEA (4.0 eq) |
| 15 | Fmoc-D-Arg(Pbf)-OH (3.00 eq) | HATU (2.85 eq) and DIEA (6.0 eq) |
| 16 | 3-azidopropanoic acid (3.00 eq) | HATU (2.85 eq) and DIEA (6.0 eq) |

Synthesis scale: 0.24 mmol.
20% piperidine in DMF was used for Fmoc deprotection for 30 min.
The coupling reaction was monitored by ninhydrin test.
After last coupling, the resin was washed with MeOH for 3 times, and then dried under vacuum.

### Peptide Cleavage and Purification:

Add cleavage cocktail (92.5% TFA/2.5% EDT/2.5% H₂O/2.5% TIS) to the flask containing the side chain protected peptide at room temperature and stir for 1.5 hr.
After filtration, the solution is added with cold isopropyl ether, and the peptide is precipitated collected by centrifugation (3 min at 3000 rpm).
The precipitate is washed with cold isopropyl ether for two additional times.
Dry the crude peptide under vacuum for 2 hr.
Dissolve the crude peptide in ACN/H₂O (1:1, 200 mL in total)
The disulfide bond is formed through I₂/MeOH, where the completion of the reaction is indicated by LCMS.
Lyophilize the reaction mixture to get the crude peptide.
Purify the crude peptide by prep-HPLC (A: 0.075% TFA in H₂O, B: ACN) to give the compound 2 (15.0 mg).

A mixture of compound 3 (6.0 mg, 1.0 eq) and compound 2 (4.93 mg, 1.0 eq) was dissolved in DMF (0.5 mL), and the reaction was stirred at 20° C. for 8 hr. LCMS showed the reaction was complete, and then the mixture was directly purified by prep-HPLC. Fractions with desired m/z (e.g., 1170.4, 939.4, 783.0, etc.) were combined and lyophilized to produce I-18 (7.1 mg, 64.9% yield, 96.1% purity) as a white solid. Purification conditions:

Separation condition

| | |
|---|---|
| Sample Preparation | Dissolve in DMF/H₂O |
| Instrument | Gilson GX-215 |
| Mobile Phase | A: H₂O (0.075% TFA in H₂O) |
| | B: CH₃CN |
| Gradient | 25-55%-60 min. Retention time: 52 min |
| Column | Luna C18, 25*200 mm, 10 um, 110A + Gemin C18, 150*30 mm, 5 um, |
| | 110A |
| Flow Rate | 20 mL/min |
| Wavelength | 220/254 nm |
| Oven Tem. | Room temperature |

Exemplary Preparation of I-19.

Exemplary Peptide Synthesis. Peptide were synthesized using standard Fmoc chemistry, for example:

1) Add DCM to the vessel containing CTC Resin (0.500 mmol, 500 mg, 1.00 mmol/g) and Fmoc-Thr(tBu)-OH (159 mg, 0.400 mmol, 0.80 eq) with N₂ bubbling.
2) Add DIEA (4.00 eq) dropwise and mix for 2 hours.

### 3) Add MeOH (0.50 mL) and mix for 30 min.

4) Drain and wash with DMF for 5 times.
5) Add 20% piperidine/DMF and react on 30 min.
6) Drain and wash with DMF for 5 times.
7) Add Fmoc-amino acid solution and mix for 30 sec first, then add activation solution, and the coupling reaction lasts for 1 hr with continuous N₂ bubbling.
8) Repeat step 4 to 7 for next amino acid coupling.

| # | Materials | Coupling reagents |
|---|---|---|
| 1 | Fmoc-Thr(tBu)-OH (0.80 eq) | DIEA (4.00 eq) |
| 2 | Fmoc-Cys(Trt)-OH (3.00 eq) | HBTU (2.85 eq) and DIEA (6.00 eq) |
| 3 | Fmoc-Trp(Boc)-OH (3.00 eq) | HBTU (2.85 eq) and DIEA (6.00 eq) |
| 4 | Fmoc-Val-OH (3.00 eq) | HBTU (2.85 eq) and DIEA (6.00 eq) |
| 5 | Fmoc-Leu-OH (3.00 eq) | HBTU (2.85 eq) and DIEA (6.00 eq) |
| 6 | Fmoc-Glu(OtBu)-OH (3.00 eq) | HBTU (2.85 eq) and DIEA (6.00 eq) |
| 7 | Fmoc-Gly-OH (3.00 eq) | HBTU (2.85 eq) and DIEA (6.00 eq) |
| 8 | Fmoc-Leu-OH (3.00 eq) | HBTU (2.85 eq) and DIEA (6.00 eq) |
| 9 | Fmoc-His(Trt)-OH (3.00 eq) | HATU (2.85 eq) and DIEA (6.00 eq) |
| 10 | Fmoc-Trp(Boc)-OH (3.00 eq) | HATU (2.85 eq) and DIEA (6.00 eq) |
| 11 | Fmoc-Ala-OH (3.00 eq) | HATU (2.85 eq) and DIEA (6.00 eq) |
| 12 | Fmoc-Cys(Trt)-OH (3.00 eq) | HATU (2.85 eq) and DIEA (6.00 eq) |
| 13 | Fmoc-Asp(OtBu)-OH (3.00 eq) | HATU (2.85 eq) and DIEA (6.00 eq) |
| 14 | Fmoc-PEG8-CH₂CH₂COOH (2.00 eq) | HATU (1.9 eq) and DIEA (4.0 eq) |
| 15 | FMOC-O-(BENZYLPHOSPHO)-SERINE (3.00 eq) | HATU (2.85 eq) and DIEA (6.0 eq) |
| 16 | FMOC-O-(BENZYLPHOSPHO)-SERINE (6.00 eq) | HATU (5.70 eq) and DIEA (12.00 eq) |
| 20 | Fmoc-Gly-OH (3.00 eq) | HATU (2.85 eq) and DIEA (6.00 eq)) |
| 21 | Fmoc-piperidine-4-carboxylic acid (3.00 eq) | HATU (2.85 eq) and DIEA (6.00 eq) |

Synthesis scale: 0.4 mmol.
20% piperidine in DMF was used for Fmoc deprotection for 30 min.
The coupling reaction was monitored by ninhydrin test.
After last amino acid coupling, N-terminal Fmoc was removed, and the resin was washed with MeOH for 3 times, and then dried under vacuum.

### Peptide Cleavage and Purification:

Add cleavage cocktail (95% TFA/2.5% EDT/2.5% H₂O/2.5% TIS) to the flask containing the side chain protected peptide at room temperature and stir for 2 hr.
The peptide is precipitated with cold isopropyl ether and collected by centrifugation (3 min at 3000 rpm).
The precipitate is washed with cold isopropyl ether for two additional times.
Dry the crude peptide under vacuum for 2 hr.
Dissolve the crude peptide in ACN/H₂O (1:1, 600 mL in total)
Adjust pH to 8 by NaHCO₃ and stir for 16 hr, and the first disulfide bond is formed through air oxidation, where the completion of the reaction is indicated by LCMS.
Lyophilize the reaction mixture to get the crude peptide.
Purify the crude peptide by prep-HPLC (TFA condition) to give the compound 2 (80.0 mg).

A mixture of compound 2 (80.0 mg, 1.0 eq) and BCN-NHS (10.8 mg, 1.1 eq) was dissolved in DMF (1 mL), and then DIEA (6.00 eq) was added slowly. The mixture was stirred at 20° C. for 8 hr. LCMS showed the reaction was complete. The mixture was then directly purified by prep-HPLC (TFA condition), and compound 3 (27.0 mg, 31.4% yield) was obtained as a white solid.

A mixture of compound 3 (27.0 mg, 1.0 eq) and compound 75 (21.2 mg, 1.0 eq) was dissolved in DMF (1.0 mL), and the reaction was stirred at 20° C. for 8 hr. LCMS showed the reaction was complete, and then the mixture was directly purified by prep-HPLC. Fractions with desired m/z (e.g., 1136.3, 909.4, 757.9, etc.) were combined and lyophilized to produce I-19 (28.8 mg, 59.7% yield, 90.4% purity) as a white solid. Purification conditions:

Separation condition

| | |
|---|---|
| Sample Preparation | Dissolve in DMF/H₂O |
| Instrument | Gilson GX-215 |
| Mobile Phase | A: H₂O (0.075% TFA in H₂O) |
| | B: CH₃CN |
| Gradient | 20-50%-60 min. Retention time: 48 min |
| Column | Luna C18, 25*200 mm, 10 um, 110A + Gemin C18, 150*30 mm, 5 um, |
| | 110A |
| Flow Rate | 20 mL/min |
| Wavelength | 220/254 nm |
| Oven Tem. | Room temperature |

Exemplary Preparation of I-24.

Exemplary Peptide Synthesis. Peptide were synthesized using standard Fmoc chemistry, for example:

1) Add DCM to the vessel containing CTC Resin (2.00 mmol, 2.00 g, 1.00 mmol/g) and Fmoc-Cys(Trt)-OH (938 mg, 1.60 mmol, 0.80 eq) with N2 bubbling.
2) Add DIEA (4.00 eq) dropwise and mix for 2 hours.

### 3) Add MeOH (0.10 mL) and mix for 30 min.

4) Drain and wash with DMF for 5 times.
5) Add 20% piperidine/DMF and react on 30 min.
6) Drain and wash with DMF for 5 times.
7) Add Fmoc-amino acid solution and mix for 30 sec first, then add activation solution, and the coupling reaction lasts for 1 hr with continuous N2 bubbling.
8)Repeat step 4 to 7 for next amino acid coupling.

| # | Materials | Coupling reagents |
|---|---|---|
| 1 | Fmoc-Cys(Trt)-OH (3.00 eq) | DIEA (4.00 eq) |
| 2 | Fmoc-Thr(tBu)-OH (3.00 eq) | HBTU (2.85 eq) and DIEA (6.00 eq) |
| 3 | Fmoc-Cys(Acm)-OH (3.00 eq) | HBTU (2.85 eq) and DIEA (6.00 eq) |
| 4 | Fmoc-Trp(Boc)-OH (3.00 eq) | HBTU (2.85 eq) and DIEA (6.00 eq) |
| 5 | Fmoc-Val-OH (3.00 eq) | HBTU (2.85 eq) and DIEA (6.00 eq) |
| 6 | Fmoc-Leu-OH (3.00 eq) | HBTU (2.85 eq) and DIEA (6.00 eq) |
| 7 | Fmoc-Glu(OtBu)-OH (3.00 eq) | HBTU (2.85 eq) and DIEA (6.00 eq) |
| 8 | Fmoc-Gly-OH (3.00 eq) | HBTU (2.85 eq) and DIEA (6.00 eq) |
| 9 | Fmoc-Leu-OH (3.00 eq) | HBTU (2.85 eq) and DIEA (6.00 eq) |
| 10 | Fmoc-His(Trt)-OH (3.00 eq) | HATU (2.85 eq) and DIEA (6.00 eq) |
| 11 | Fmoc-Trp(Boc)-OH (3.00 eq) | HATU (2.85 eq) and DIEA (6.00 eq) |
| 12 | Fmoc-Ala-OH (3.00 eq) | HATU (2.85 eq) and DIEA (6.00 eq) |
| 13 | Fmoc-Cys(Acm)-OH (3.00 eq) | HATU (2.85 eq) and DIEA (6.00 eq) |
| 14 | Fmoc-Asp(OtBu)-OH (3.00 eq) | HATU (2.85 eq) and DIEA (6.00 eq) |
| 15 | Fmoc-Cys(Trt)-OH (3.00 eq) | HATU (2.85 eq) and DIEA (6.00 eq) |

Synthesis scale: 1.6 mmol
20% piperidine in DMF was used for Fmoc deprotection for 30 min.
The coupling reaction was monitored by ninhydrin test.
After last amino acid coupling, N-terminal Fmoc was removed, and the resin was washed with MeOH for 3 times, and then dried under vacuum.

### Peptide Cleavage and Purification:

Add cleavage solution (20% HFIP/DCM) to the flask containing the side chain protected peptide at room temperature. The cleavage was carried out twice (20 min each), with continuous N₂ bubbling.
After filtered, the filtrate was concentrated under reduced pressure and the residue was dried in lyopholizer to give compound 1 (3.1 g, crude) as a white solid.

Another example:

1) Add DCM to the vessel containing CTC Resin (2.00 mmol, 2.00 g, 1.00 mmol/g) and Hexane-1,6-diamine (256 mg, 2.20 mmol, 1.10 eq) with N₂ bubbling.
2) Add DIEA (4.00 eq) dropwise and mix for 2 hours.

### 3) Add MeOH (2.00 mL) and mix for 30 min.

4) Drain and wash with DMF for 5 times.
5) Add 20% piperidine/DMF and react for 30 min.
6) Drain and wash with DMF for 5 times.
7) Add Fmoc-amino acid solution and mix for 30 sec first, then add activation solution, and the coupling reaction lasts for 1 hr with continuous N₂ bubbling.
8) Repeat above step 4 to 7 for the coupling of following amino acids.

| # | Materials | Coupling reagents |
|---|---|---|
| 1 | Fmoc-Ahx-OH (3.00 eq) | DIEA (4.00 eq) |
| 2 | Fmoc-Gly-Gly-OH (3.00 eq) | HATU (2.85 eq) and DIEA (6.00 eq) |
| 3 | Fmoc-D-gamaGlu(OtBu)-OH (3.00 eq) | HATU (2.85 eq) and DIEA (6.00 eq) |
| 4 | Fmoc-D-gamaGlu(OtBu)-OH (3.00 eq) | HATU (2.85 eq) and DIEA (6.00 eq) |
| 5 | Fmoc-D-Glu(OtBu)-OH (3.00 eq) | HATU (2.85 eq) and DIEA (6.00 eq) |
| 6 | Fmoc-D-gamaGlu(OtBu)-OH (3.00 eq) | HATU (2.85 eq) and DIEA (6.00 eq) |
| 7 | Fmoc-D-gamaGlu(OtBu)-OH (3.00 eq) | HATU (2.85 eq) and DIEA (6.00 eq) |
| 8 | Fmoc-Gly-OH (3.00 eq) | HATU (2.85 eq) and DIEA (6.00 eq) |
| 9 | Fmoc-piperidine-4-carboxylic acid (3.00 eq) | HATU (2.85 eq) and DIEA (6.00 eq) |

Synthesis scale: 2.0 mmol.
20% piperidine in DMF was used for Fmoc deprotection for 30 min.
The coupling reaction was monitored by ninhydrin test.
After last amino acid coupling, N-terminal Fmoc was removed, and the resin was washed with MeOH for 3 times, and then dried under vacuum.

### Peptide Cleavage and Purification:

Add cleavage solution (20% HFIP/DCM) to the flask containing the side chain protected peptide at room temperature. The cleavage was carried out twice (20 min each), with continuous N₂ bubbling.
After filtered, the filtrate was concentrated under reduced pressure and the residue was dried in lyophilizer to give compound 2 (800 mg, crude) as a white solid.

A mixture of compound 2 (500 mg, 1.0 eq) and compound 1 (1.35 g, 1.2 eq) was dissolved in DMF (5.0 mL) first, following by addition of HOAt (2.0 eq, pre-dissolved in DMF) and DIC (2.0 eq). The mixture was stirred at 30° C. for 8 hr, till LCMS showed the coupling was complete. The mixture was concentrated under reduced pressure to remove solvent. The resulting crude was added with 30 mL of deprotection cocktail (95% TFA/2.5% TIS/2.5% H₂O/2.5% EDT) to remove all protecting groups, and this reaction lasted 2 hr with continuous stirring at room temperature (15-25° C.). The peptide was then precipitated with cold tert-butyl methyl ether (150 mL) and centrifuged (3 min at 3000 rpm). The precipitates were collected and washed with cold tert-butyl methyl ether with two additional times (150 mL each), and the resulting crude peptide was then dried under vacuum for 2 hr. The deprotected peptide exposed two Cys, which were designed to form first disulfide bond by free oxidation. Therefore, the crude peptide was then dissolved in ACN/H₂O (300 mL), added with 1 M NaHCO₃ till pH reached 8, and the resulting solution was finally stirred for 16 hr with air supplement. LCMS indicated that the first disulfide bond was formed, and then the solution was lyophilized to dry. The residue was directly purified by prep-HPLC (TFA condition) to give compound 5 (150 mg, 13.7% yield) as a white solid.

Compound 5 (150 mg) was dissolved in ACN/H₂O (50.0 mL), and then 1 M HCl was added to adjust pH to 1, following by dropwise addition of 0.1 M I₂/AcOH till the mixture turned brown. The mixture was then stirred at 20° C. for 10 hr. LCMS showed the reaction was complete. The mixture was purified by prep-HPLC (TFA condition), resulting compound 6 (70 mg, 49.0% yield) obtained as a white solid.

Compound 6 (70.0 mg, 1.0 eq) and BCN-NHS (7.50 mg, 1.1 eq) were dissolved in DMF (3.00 mL), and then DIEA (6.00 eq) was added slowly. The mixture was stirred at 30° C. for 8 hours. LCMS indicated that the coupling reaction was complete. Furthermore, 20% piperidine in DMF was added for Fmoc removal (30 min). The mixture was directly purified by prep-HPLC, and compound 7 (20.0 mg) was obtained as a white solid.

A mixture of compound 7 (20.0 mg, 1.0 eq) and compound 75 (13.6 mg, 1.0 eq) was dissolved in DMF (1.0 mL), and the mixture was stirred at 15° C. for 8 hr. When the click reaction was complete, the mixture was purified by prep-HPLC. Fractions with desired m/z (e.g., 1644.9, 1234.2, 987.6, 823.2, etc.) were lyophilized to produce I-24 (18.1 mg, 53.9% yield, 98.3% purity) as a white solid. Purification condition:

Separation condition

| | |
|---|---|
| Sample Preparation | Dissolve in DMF/H₂O |
| Instrument | Gilson GX-215 |
| Mobile Phase | A: H₂O (0.075% TFA in H₂O) |
| | B: CH₃CN |
| Gradient | 20-50%-60 min. Retention time: 54 min |
| Column | Luna C18, 25*200 mm, 10 um, 110A + Gemin C18, 150*30 mm, 5 um, |
| | 110A |
| Flow Rate | 20 mL/min |
| Wavelength | 220/254 nm |
| Oven Tem. | Room temperature |

Exemplary Preparation of I-25, I-26, I-28, and I-29.

Exemplary Peptide Synthesis. Peptide were synthesized using standard Fmoc chemistry, for example:

1) Resin preparation: To the vessel containing CTC Resin (0.3 mmol, 0.30 g, 1.0 mmol/g) and Fmoc-Thr(tBu)-OH (0.119 g, 0.30 mmol, 1.00 eq) in DCM (5 mL) was added DIEA (4.00 eq) dropwise and mix for 2 hr with N₂ bubbling at 15° C. Then added MeOH (0.3 mL) and bubbled with N₂ for another 30 min. The resin was washed with DMF (10 mL)*5. Then 20% piperidine in DMF (10 mL) was added and the mixture was bubbled with N₂ for 30 min at 15° C. Then the mixture was filtered to obtain the resin. The resin was washed with DMF (10 mL)*5 before proceeding to next step.
2) Coupling: A solution of Fmoc-Cys(Trt)-OH (3.00 eq), HBTU (2.85 eq) in DMF (5 mL) was added to the resin with N₂ bubbling. Then DIEA (6.00 eq) was added to the mixture dropwise and bubbled with N₂ for 30 min at 15° C. The coupling reaction was monitored by ninhydrin test, if it showed colorless, the coupling was completed. If it showed blue or brownish red, the coupling was repeated and then checked with ninhydrin test again till reaching completion. The resin was then washed with DMF (10 mL)*5.
3) De-protection: 20% piperidine in DMF (10 mL) was added to the resin and the mixture was bubbled with N₂ for 30 mins at 15° C. The resin was then washed with DMF (10 mL)*5. The de-protection reaction was monitored by ninhydrin test, if it showed blue or other brownish red, the reaction was completed.
4) Repeat Step 2 and 3 for all other amino acids: see below.
5) After the last position completed, the resin was washed with DMF (10 mL)*5, MEOH (10 mL)*5 and then dried under vacuum.

| # | Materials | Coupling reagents |
|---|---|---|
| 1 | Fmoc-Thr(tBu)-OH (1.00 eq) | DIEA (4.00 eq) |
| 2 | Fmoc-Cys(stBu)-OH (2.00 eq) | HATU (1.9 eq) and DIEA (4.00 eq) |
| 3 | Fmoc-Trp-OH (3.00 eq) | HBTU (2.85 eq) and DIEA (6.00 eq) |
| 4 | Fmoc-Val-OH (3.00 eq) | HBTU (2.85 eq) and DIEA (6.00 eq) |
| 5 | Fmoc-Leu-OH (3.00 eq) | HBTU (2.85 eq) and DIEA (6.00 eq) |
| 6 | Fmoc-Glu(OtBu)-OH (3.00 eq) | HBTU (2.85 eq) and DIEA (6.00 eq) |
| 7 | Fmoc-Gly-OH (3.00 eq) | HBTU (2.85 eq) and DIEA (6.00 eq) |
| 8 | Fmoc-Leu-OH (3.00 eq) | HBTU (2.85 eq) and DIEA (6.00 eq) |
| 9 | Fmoc-His(Trt)-OH (2.00 eq) | HBTU (2.85 eq) and DIEA (6.00 eq) |
| 10 | Fmoc-Trp-OH (3.00 eq) | HBTU (2.85 eq) and DIEA (6.00 eq) |
| 11 | Fmoc-Ala-OH (3.00 eq) | HBTU (2.85 eq) and DIEA (6.00 eq) |
| 12 | Fmoc-Cys(stBu)-OH (2.00 eq) | HATU (1.9 eq) and DIEA (4.00 eq) |
| 13 | Fmoc-Asp(OtBu)-OH (3.00 eq) | HBTU (2.85 eq) and DIEA (6.00 eq) |
| 14 | Fmoc-Gly-Gly-OH (3.00 eq) | HATU (2.85 eq) and DIEA (6.00 eq) |
| 15 | Fmoc-D-Glu-OtBu (3.00 eq) | HATU (2.85 eq) and DIEA (6.00 eq) |
| 16 | Fmoc-D-Glu-OtBu (3.00 eq) | HATU (2.85 eq) and DIEA (6.00 eq) |
| 17 | (R)-2-((((9H-fluoren-9-yl)methoxy)carbonyl)amino)-5- | HATU (1.9 eq) and DIEA (4.00 eq) |
| | ((12-(tert-butoxy)-12-oxododecyl)amino)-5- | |
| | oxopentanoic acid (2.00 eq) | |
| 18 | Fmoc-D-Glu-OtBu (3.00 eq) | HATU (2.85 eq) and DIEA (6.00 eq) |
| 19 | Fmoc-D-Glu-OtBu (3.00 eq) | HATU (2.85 eq) and DIEA (6.00 eq) |
| 20 | Fmoc-PEG₈-CH₂CH₂CH₂OH (1.5 eq) | DIEA (4.00 eq) |
| 21 | Fmoc-Cys(Mmt)-OH (2.00 eq) | HATU (1.90 eq) and DIEA (4.00 eq) |
| 22 | Fmoc-Asp(OtBu)-OH (3.00 eq) | HBTU (2.85 eq) and DIEA (6.00 eq) |
| 23 | (S)-2-((((9H-fluoren-9- | HATU (1.90 eq) and DIEA (4.00 eq) |
| | yl)methoxy)carbonyl)amino)heptanoic acid (2.00 eq) | |
| 24 | (S)-2-((((9H-fluoren-9-yl)methoxy)carbonyl)amino)-3- | HATU (1.90 eq) and DIEA (4.00 eq) |
| | ([1,1′-biphenyl]-4-yl)propanoic acid (2.00 eq) | |
| 25 | Fmoc-Leu-OH (3.00 eq) | HATU (2.85 eq) and DIEA (6.00 eq) |
| 26 | Fmoc-Val-OH (3.00 eq) | HATU (2.85 eq) and DIEA (6.00 eq) |
| 27 | Fmoc-Gly-OH (3.00 eq) | HATU (2.85 eq) and DIEA (6.00 eq) |
| 28 | Fmoc-Asp(OtBu)-OH (3.00 eq) | HATU (2.85 eq) and DIEA (6.00 eq) |
| 29 | Fmoc-His(Trt)-OH (3.00 eq) | HATU (2.85 eq) and DIEA (6.00 eq) |
| 30 | Fmoc-Tyr(tBu)-OH (1.50 eq) | HATU (2.85 eq) and DIEA (6.00 eq) |
| 31 | (S)-2-((((9H-fluoren-9- | HATU (1.90 eq) and DIEA (4.00 eq) |
| | yl)methoxy)carbonyl)amino)heptanoic acid (2.00 eq) | |
| 32 | Fmoc-Arg(Pbf)-OH (3.00 eq) | HATU (2.85 eq) and DIEA (6.00 eq) |
| 33 | Fmoc-Ala-OH (3.00 eq) | HATU (2.85 eq) and DIEA (6.00 eq) |
| 34 | 2-Bromoacetic acid (6.00 eq) | DIC (6.00 eq) |

### Peptide Cleavage and Purification:

1) Add cleavage buffer (92.5% TFA/2.5% TIS/2.5% H₂O/2.5%3-mercaptopropanoic acid) to the flask containing the sidechain protected peptide at room temperature and stirred for 1 hr.
2) Filter and collect the filtrate.
3) The peptide is precipitated with cold isopropyl ether (100 mL) and centrifuged (3 min at 3000 rpm).
4) Cold isopropyl ether washes the precipitate two additional times, and dry the crude peptide under vacuum for 2 hr.
5) To a mixture of the crude in ACN/H₂O (300 mL) was added NaHCO₃ to adjust pH to 8, then the mixture was stirred at 15° C. for 30 min. The mixture was quenched with 1 M HCl to adjust pH to 7. The mixture was dried via lyophilization, and the residue was purified by prep-HPLC (TFA condition) directly to get compound 2 (50 mg, 90% purity).

To a mixture of compound 2 (50 mg, 10.5 ummol, 1.0 eq) in MeCN/H₂O (1/1, 3 mL) was added TCEP (12.0 mg, 42.0 umol, 4.0 eq), based with saturated aqueous NaHCO₃ to pH=8, then the mixture was stirred at 15° C. for 3 hrs. The mixture was purified by Flash (acid condition, TFA) directly to get compound 3 (30 mg) as a white solid.

Compound 3 (25 mg, 5.48 umol, 1.0 eq) was dissolved in ACN (10 mL) and H₂O (10 mL) at 20° C. And then adjusted pH to 8 by NaHCO₃. The mixture was stirred at 20° C. for 72 hr. LCMS showed the reaction was completed. The solution was purified by prep-HPLC (TFA condition) directly to get I-28 (5.0 mg, 18.97% yield, 94.8% purity) as a white solid. Purification conditions:

Purification condition

| | |
|---|---|
| Sample Preparation | Dissolve in MeCN/H₂O |
| Instrument | Gilson GX-215 |
| Mobile Phase | A: H₂O (0.075% TFA in H₂O) |
| | B: MeCN |
| Gradient | 20-50%-60 min. Retention time: 50 min |
| Column | Luna 25*200 mm, C 18 10 um, 110 A + Gemin 150*30 mm, C18 5 um, 110 Å |
| Flow Rate | 20 mL/min |
| Wavelength | 220/254 nm |
| Oven Tem. | Room temperature |

I-25, I-26 and I-29 were prepared similarly. Certain results from certain preparations were provided below:

| Compound | LC/MS (+ESI; major peak) | Purifiy (HPLC) |
|---|---|---|
| I-25 | 1491.2 | 95% |
| I-26 | 1461.7 | 96% |
| I-28 | 1520.5 | 95% |
| I-29 | 1432.4 | 95% |

Exemplary Preparation of I-27.

Exemplary Peptide Synthesis. Peptide were synthesized using standard Fmoc chemistry, for example:

1) Resin preparation: To the vessel containing CTC Resin (0.5 mmol, 0.50 g, 1.0 mmol/g) and Fmoc-Thr(tBu)-OH (0.198 g, 0.50 mmol, 1.00 eq) in DCM (5 mL) was added DIEA (4.00 eq) dropwise and mix for 2 hr with N₂ bubbling at 15° C. Then added MeOH (0.5 mL) and bubbled with N₂ for another 30 min. The resin was washed with DMF (10 mL)*5. Then 20% piperidine in DMF (10 mL) was added and the mixture was bubbled with N₂ for 30 min at 15° C. Then the mixture was filtered to obtain the resin. The resin was washed with DMF (10 mL)*5 before proceeding to next step.
2) Coupling: A solution of Fmoc-Cys(Trt)-OH (3.00 eq), HBTU (2.85 eq) in DMF (5 mL) was added to the resin with N₂ bubbling. Then DIEA (6.00 eq) was added to the mixture dropwise and bubbled with N₂ for 30 min at 15° C. The coupling reaction was monitored by ninhydrin test, if it showed colorless, the coupling was complete. If it showed blue or brownish red, the coupling was repeated and then checked with ninhydrin test again till reaching completion. The resin was then washed with DMF (10 mL)*5.
3) De-protection: 20% piperidine in DMF (10 mL) was added to the resin and the mixture was bubbled with N₂ for 30 min at 15° C. The resin was then washed with DMF (10 mL)*5. The De-protection reaction was monitored by ninhydrin test, if it showed blue or other brownish red, the reaction was completed.
4) Repeat Step 2 and 3 for all other amino acids: see below.
5) After the last position completed, the resin was washed with DMF (10 mL)*5, MeOH (10 mL)*5 and then dried under vacuum.

| # | Materials | Coupling reagents |
|---|---|---|
| 1 | Fmoc-Thr(tBu)—OH (1.00 eq) | DIEA (4.00 eq) |
| 2 | Fmoc-Cys(Trt)-OH (3.00 eq) | HBTU (2.85 eq) and DIEA(6.00 eq) |
| 3 | Fmoc-Trp-OH (3.00 eq) | HBTU (2.85 eq) and DIEA(6.00 eq) |
| 4 | Fmoc-Val-OH (3.00 eq) | HBTU (2.85 eq) and DIEA(6.00 eq) |
| 5 | Fmoc-Leu-OH (3.00 eq) | HBTU (2.85 eq) and DIEA(6.00 eq) |
| 6 | Fmoc-Glu(OtBu)—OH (3.00 eq) | HBTU (2.85 eq) and DIEA(6.00 eq) |
| 7 | Fmoc-Gly-OH (3.00 eq) | HBTU (2.85 eq) and DIEA(6.00 eq) |
| 8 | Fmoc-Leu-OH (3.00 eq) | HBTU (2.85 eq) and DIEA(6.00 eq) |
| 9 | Fmoc-His(Trt)-OH (2.00 eq) | HBTU (2.85 eq) and DIEA(6.00 eq) |
| 10 | Fmoc-Trp-OH (3.00 eq) | HBTU (2.85 eq) and DIEA(6.00 eq) |
| 11 | Fmoc-Ala-OH (3.00 eq) | HBTU (2.85 eq) and DIEA(6.00 eq) |
| 12 | Fmoc-Cys(Trt)-OH (3.00 eq) | HBTU (2.85 eq) and DIEA(6.00 eq) |
| 13 | Fmoc-Asp(OtBu)—OH (3.00 eq) | HBTU (2.85 eq) and DIEA(6.00 eq) |
| 14 | Fmoc-PEG₆-CH₂CH₂COOH (2.00 eq) | HATU (1.90 eq) and DIEA(4.00 eq) |
| 15 | (R)-2-((((9H-fluoren-9-yl)methoxy)carbonyl)amino)-5- | HATU (1.90 eq) and DIEA(4.00 eq) |
| | ((12-(tert-butoxy)-12-oxododecyl)amino)-5- | |
| | oxopentanoic acid (1.50 eq) | |

### Peptide Cleavage and Purification:

1) Add cleavage buffer (95% TFA/2.5% TIS/2.5% H₂O, 10 mL) to the flask containing the sidechain protected peptide at room temperature and stirred for 1 hr.
2) Filter and collect the filtrate.
3) The peptide is precipitated with cold isopropyl ether (50 mL) and centrifuged (3 min at 3000 rpm).
4) Isopropyl ether washes two additional times, and dry the crude peptide under vacuum for 2 hr.
5) To give compound 1 (800 mg, crude) as a white solid.

To a mixture of compound 1 in MeCN/H₂O (500 mL) was added 0.1 M I₂/AcOH dropwise until the light yellow persisted, then the mixture was stirred at 15° C. for 5 min. The mixture was quenched with 0.1 M Na₂S₂O₃ dropwise until the light yellow disappeared. The mixture was dried via lyophilization. The residue was purified by prep-HPLC (TFA condition) directly to get compound 2 (200 mg, 90.0% purity, 18.2% yield).

Another exemplary Peptide Synthesis. Peptide were synthesized using standard Fmoc chemistry, for example:

1) Resin preparation: To the vessel containing CTC Resin (1.0 mmol, 1.0 g, 1.0 mmol/g) and Fmoc-PEG₁₀-CH₂CH₂COOH (0.75 g, 1.0 mmol, 1.00 eq) in DCM (10 mL) was added DIEA (4.00 eq) dropwise and mix for 2 hr with N₂ bubbling at 15° C. Then added MeOH (1.0 mL) and bubbled with N₂ for another 30 min. The resin was washed with DMF (20 mL)*5. Then 20% piperidine in DMF (20 mL) was added and the mixture was bubbled with N₂ for 30 min at 15° C. Then the mixture was filtered to obtain the resin. The resin was washed with DMF (20 mL)*5 before proceeding to next step.
2) Coupling: A solution of Fmoc-Cys(Mmt)-OH (2.00 eq), HBTU (1.90 eq) in DMF (5 mL) was added to the resin with N₂ bubbling. Then DIEA (4.00 eq) was added to the mixture dropwise and bubbled with N₂ for 30 min at 15° C. The coupling reaction was monitored by ninhydrin test, if it showed colorless, the coupling was complete. If it showed blue or brownish red, the coupling was repeated and then checked with ninhydrin test again till reaching completion. The resin was then washed with DMF (10 mL)*5.
3) De-protection: 20% piperidine in DMF (10 mL) was added to the resin and the mixture was bubbled with N₂ for 30 min at 15° C. The resin was then washed with DMF (10 mL)*5. The De-protection reaction was monitored by ninhydrin test, if it showed blue or other brownish red, the reaction was complete.
4) Repeat Step 2 and 3 for all other amino acids: see below.
5) Coupling for the last position: A solution of 2-bromoacetic acid (4.00 eq) and DIC (4.00 eq) was added to resin and the mixture was bubbled with N₂ for 20 min. The coupling reaction was monitored by ninhydrin test, if it showed colorless, the coupling was complete. The resin was then washed with DMF (10 mL)*5, MeOH (10 mL)*5, and then dried under vacuum.

| # | Materials | Coupling reagents |
|---|---|---|
| 1 | Fmoc-PEG₁₀-CH₂CH₂CH₂OH (1.00 eq) | DIEA (4.00 eq) |
| 2 | Fmoc-Cys(Mmt)-OH (2.00 eq) | HATU (1.90 eq) and DIEA(4.00 eq) |
| 3 | Fmoc-Asp(OtBu)—OH (3.00 eq) | HBTU (2.85 eq) and DIEA(6.00 eq) |
| 4 | (S)-2-((((9H-fluoren-9- | HATU (1.35 eq) and DIEA(3.00 eq) |
| | yl)methoxy)carbonyl)amino)heptanoic acid (1.50 eq) | |
| 5 | (S)-2-((((9H-fluoren-9-yl)methoxy)carbonyl)amino)-3- | HATU (1.35 eq) and DIEA(3.00 eq) |
| | ([1,1′-biphenyl]-4-yl)propanoic acid (1.50 eq) | |
| 6 | Fmoc-Leu-OH (3.00 eq) | HBTU (2.85 eq) and DIEA(6.00 eq) |
| 7 | Fmoc-Val-OH (3.00 eq) | HBTU (2.85 eq) and DIEA(6.00 eq) |
| 8 | Fmoc-Gly-OH (3.00 eq) | HBTU (2.85 eq) and DIEA(6.00 eq) |
| 9 | Fmoc-Asp(OtBu)—OH (3.00 eq) | HBTU (2.85 eq) and DIEA(6.00 eq) |
| 10 | Fmoc-His(Trt)-OH (3.00 eq) | HBTU (2.85 eq) and DIEA(6.00 eq) |
| 11 | Fmoc-Tyr(tBu)—OH (1.50 eq) | HBTU (2.85 eq) and DIEA(6.00 eq) |
| 12 | (S)-2-((((9H-fluoren-9- | HATU (1.35 eq) and DIEA(3.00 eq) |
| | yl)methoxy)carbonyl)amino)heptanoic acid (1.50 eq) | |
| 13 | Fmoc-Arg(Pbf)-OH (3.00 eq) | HBTU (2.85 eq) and DIEA(6.00 eq) |
| 14 | Fmoc-Ala-OH (3.00 eq) | HBTU (2.85 eq) and DIEA(6.00 eq) |
| 15 | 2-Bromoacetic acid (4.00 eq) | DIC (4.00 eq) |

### Peptide Cleavage and Purification:

1) Add cleavage buffer (2% TFA/2% TIS/96% DCM, 100 mL) to the flask containing the sidechain protected peptide at room temperature with N₂ bubbling for 20 min.
2) Filter and collect the filtrate. The clear solution is compound 3 (1.0 mmol) in cleavage buffer (100 mL), and used in next step directly.

Compound 3 (1.0 mmol, 100 mL, in cleavage buffer) was diluted with MeOH (1000 mL), based with TEA to pH=8 under N₂ atmosphere, then stirred at 15° C. for 2 hr. The solvent was removed under reduced pressure, and the residue was triturated in 0.1 M HCl (cold, 100 mL). After filtration, the solid was washed with H₂O (50 mL), and then was stirred in isopropyl ether (50 mL) for 10 mins. The solid was finally dried under reduced pressure to get compound 4 (1.80 g, crude).

A mixture of compound 4 (1.80 g, 0.63 mmol, 1.00 eq), compound 4a (522 mg, 3.15 mmol, 5.00 eq), EDCI (361 mg, 1.89 mmol, 3.00 eq) in DMF (10 mL) was stirred at 15° C. for 3 hrs. The mixture was added to 0.5 M HCl (cold, 100 mL), there appeared lots of white solid. After filtration, the solid was washed with H₂O (20 mL), dried via lyophilization to get compound 5 (2.0 g, crude) as a white solid.

A mixture of compound 5 (2.0 g, crude) was dissolved in a mixture solution containing TFA (45.6 g, 400 mmol, 30 mL), H₂O (0.75 g, 41.6 mmol, 0.75 mL) and triisopropylsilane (0.58 g, 3.67 mmol, 0.75 mL), and was stirred at 15° C. for 1 hr. The mixture was precipitated with cold isopropyl ether (100 mL) and centrifuged (3 min at 3000 rpm). Cold isopropyl ether (50 mL) washed the precipitate two additional times. Dry the crude peptide under vacuum for 2 hr, and then the residue was purified by flash C18 (ISCO®; 120 g SepaFlash® C18 Flash Column, Eluent of 0-90% MeCN/H₂O gradient @ 75 mL/min) directly to get compound 6 (180 mg, 77.4 umol, 11.5% yield, 90.0% purity) as a white solid.

A mixture of compound 6 (80.0 mg, 34.4 umol, 1.00 eq), compound 2 (75.0 mg, 34.4 umol, 1.00 eq), DIEA (35.5 mg, 275 umol, 47.9 uL, 8.00 eq) in DMF (0.5 mL) was stirred at 15° C. for 1 hr. The solution was purified by prep-HPLC (TFA condition) directly to get compound I-27 (67.0 mg, 15.3 umol, 44.4% yield, 95.3% purity) as a white solid. LCMS: 1451.6 (+ESI Scan; major peak).

Purification Conditions:

Purification condition

| Sample Preparation | Dissolve in MeCN/H₂O |
|---|---|
| Instrument | Gilson GX-215 |
| Mobile Phase | A: H₂O (0.075% TFA in H₂O) |
| | B: MeCN |
| Gradient | 30-60%-60 min. Retention time: 40 min |
| Column | Luna25*200 mm, C18 10 um, 110 A + |
| | Gemin150*30 mm, C18 5 um, 110 Å |
| Flow Rate | 20 mL/min |
| Wavelength | 220/254 nm |
| Oven Tem. | Room temperature |

Exemplary Preparation of I-30 and I-31.

Exemplary Peptide Synthesis. Peptide were synthesized using standard Fmoc chemistry, for example:

1) Resin preparation: To the vessel containing Rink Amide MBHA Resin (0.2 mmol, 0.65 g, 0.31 mmol/g) in DMF (5 mL) with N₂ bubbling at 15° C. for 30 min. Then 20% piperidine in DMF (10 mL) was added and the mixture was bubbled with N₂ for 30 mins at 15° C. Then the mixture was filtered to obtain the resin. The resin was washed with DMF (10 mL)*5 before proceeding to next step.
2) Coupling: A solution of Fmoc-Thr(tBu)-OH (3.00 eq), HBTU (2.85 eq) in DMF (5 mL) was added to the resin with N₂ bubbling. Then DIEA (6.00 eq) was added to the mixture dropwise and bubbled with N₂ for 30 min at 15° C. The coupling reaction was monitored by ninhydrin test, if it showed colorless, the coupling was completed. If it showed blue or brownish red, the coupling was repeated and checked with ninhydrin test again till reaching completion. The resin was then washed with DMF (10 mL)*5.
3) De-protection: 20% piperidine in DMF (10 mL) was added to the resin and the mixture was bubbled with N₂ for 30 mins at 15° C. The resin was then washed with DMF (10 mL)*5. The De-protection reaction was monitored by ninhydrin test, if it showed blue or other brownish red, the reaction was completed.
4) Repeat Step 2 and 3 for all other amino acids: see below.
5) After the last position completed, the resin was washed with DMF (10 mL)*5, MeOH (10 mL)*5 and then dried under vacuum.
After cycle 27, Fmoc group was kept on resin, and then Mint group on Cys sidechain was removed by 2% TFA/2% TIS/DCM (2 min×5), and then disulfide bond was formed on resin by adding 2 eq NCS and reacted for 15 min Disulfide formation was confirmed by pilot cleavage and LCMS analysis, and then Fmoc was removed and 2-bromoacetic acid was coupled as the last residue.

| # | Materials | Coupling reagents |
|---|---|---|
| 1 | Fmoc-Thr(tBu)—OH (3.00 eq) | HBTU (2.85 eq) and DIEA(6.00 eq) |
| 2 | Fmoc-Cys(Mmt)-OH (2.00 eq) | HATU (1.9 eq) and DIEA(4.00 eq) |
| 3 | Fmoc-Trp(Boc)-OH (3.00 eq) | HBTU (2.85 eq) and DIEA(6.00 eq) |
| 4 | Fmoc-Val-OH (3.00 eq) | HBTU (2.85 eq) and DIEA(6.00 eq) |
| 5 | Fmoc-Leu-OH (3.00 eq) | HBTU (2.85 eq) and DIEA(6.00 eq) |
| 6 | Fmoc-Glu(OtBu)—OH (3.00 eq) | HBTU (2.85 eq) and DIEA(6.00 eq) |
| 7 | Fmoc-Gly-OH (3.00 eq) | HBTU (2.85 eq) and DIEA(6.00 eq) |
| 8 | Fmoc-Leu-OH (3.00 eq) | HBTU (2.85 eq) and DIEA(6.00 eq) |
| 9 | Fmoc-His(Trt)-OH (2.00 eq) | HBTU (2.85 eq) and DIEA(6.00 eq) |
| 10 | Fmoc-Trp(Boc)-OH (3.00 eq) | HBTU (2.85 eq) and DIEA(6.00 eq) |
| 11 | Fmoc-Ala-OH (3.00 eq) | HBTU (2.85 eq) and DIEA(6.00 eq) |
| 12 | Fmoc-Cys(Mmt)-OH (2.00 eq) | HATU (1.9 eq) and DIEA(4.00 eq) |
| 13 | Fmoc-Asp(OtBu)—OH | HBTU (2.85 eq) and DIEA(6.00 eq) |
| 14 | 4-((((9H-fluoren-9- | HBTU (2.85 eq) and DIEA(6.00 eq) |
| | yl)methoxy)carbonyl)amino)butanoic acid (3.00 eq) | |
| 15 | Fmoc-Cys(Trt)-OH (2.00 eq) | HATU (2.85 eq) and DIEA(6.00 eq) |
| 16 | Fmoc-Asp(OtBu)—OH (3.00 eq) | HBTU (2.85 eq) and DIEA(6.00 eq) |
| 17 | (S)-2-((((9H-fluoren-9- | HATU (1.90 eq) and DIEA(4.00 eq) |
| | yl)methoxy)carbonyl)amino)heptanoic acid (2.00 eq) | |
| 18 | (S)-2-((((9H-fluoren-9-yl)methoxy)carbonyl)amino)-3- | HATU (1.90 eq) and DIEA(4.00 eq) |
| | ([1,1′-biphenyl]-4-yl)propanoic acid (2.00 eq) | |
| 19 | Fmoc-Leu-OH (3.00 eq) | HATU (2.85 eq) and DIEA(6.00 eq) |
| 20 | Fmoc-Val-OH (3.00 eq) | HATU (2.85 eq) and DIEA(6.00 eq) |
| 21 | Fmoc-Gly-OH (3.00 eq) | HATU (2.85 eq) and DIEA(6.00 eq) |
| 22 | Fmoc-Asp(OtBu)—OH (3.00 eq) | HATU (2.85 eq) and DIEA(6.00 eq) |
| 23 | Fmoc-His(Trt)-OH (3.00 eq) | HATU (2.85 eq) and DIEA(6.00 eq) |
| 24 | Fmoc-Tyr(tBu)—OH (1.50 eq) | HATU (2.85 eq) and DIEA(6.00 eq) |
| 25 | (S)-2-((((9H-fluoren-9- | HATU (1.90 eq) and DIEA(4.00 eq) |
| | yl)methoxy)carbonyl)amino)heptanoic acid (2.00 eq) | |
| 26 | Fmoc-Arg(Pbf)-OH (3.00 eq) | HATU (2.85 eq) and DIEA(6.00 eq) |
| 27 | Fmoc-Ala-OH (3.00 eq) | HATU (2.85 eq) and DIEA(6.00 eq) |
| 28 | Disulfide bond formation on solid phase | NCS (2 eq), 15 min |
| 29 | 2-bromoacetic acid (6.00 eq) | DIC (6.00 eq) |

### Peptide Cleavage and Purification:

1) Add cleavage buffer (92.5% TFA/2.5% TIS/2.5% H₂O/2.5% 3-mercaptopropanoic acid, 10 mL) to the flask containing the side chain protected peptide at room temperature and stirred for 2 hr.
2) Filter and collect the filtrate.
3) The peptide is precipitated with cold isopropyl ether (100 mL) and centrifuged (3 mins at 3000 rpm).
4) Isopropyl ether washes the precipitate two additional times, and dry the crude peptide under vacuum for 2 hr, resulting in crude linear peptide (0.68 g, crude) as a white solid.
5) Crude linear peptide (0.68 g) was dissolved in MeCN/H₂O (200 mL), then adjusted pH to 8 by 0.2 M NaHCO₃ and stirred at 15° C. for 1 hr. The mixture was adjusted pH to 6 by 1 M HCl after the reaction was completed. The mixture was dried in lyophilization. The solution was purified by prep-HPLC (acid condition, TFA) directly to get compound 425 (6 mg, 0.89% yield, 96.6% purity) as a white solid.

### Purification Conditions:

Purification condition

| Sample Preparation | Dissolve in MeCN/H₂O |
|---|---|
| Instrument | Gilson GX-215 |
| Mobile Phase | A: H₂O (0.075% TFA in H₂O) |
| | B: MeCN |
| Gradient | 20-50%-60 min. Retention time: 50 min |
| Column | Luna 25*200 mm, C18 10 um, 110 Å + |
| | Gemin 150*30 mm, C18 5 um, 110 Å |
| Flow Rate | 20 mL/min |
| Wavelength | 220/254 nm |
| Oven Tem. | Room temperature |

I-31 were prepared similarly. Certain results from certain preparations were provided below:

| Compound | LC/MS (+ESI; major peak) | Purifiy (HPLC) |
|---|---|---|
| I-30 | 1088.3 | 91% |
| I-31 | 1113.4 | 95% |

Exemplary Preparation of I-32.

A preparation of I-32 is described below as an example.

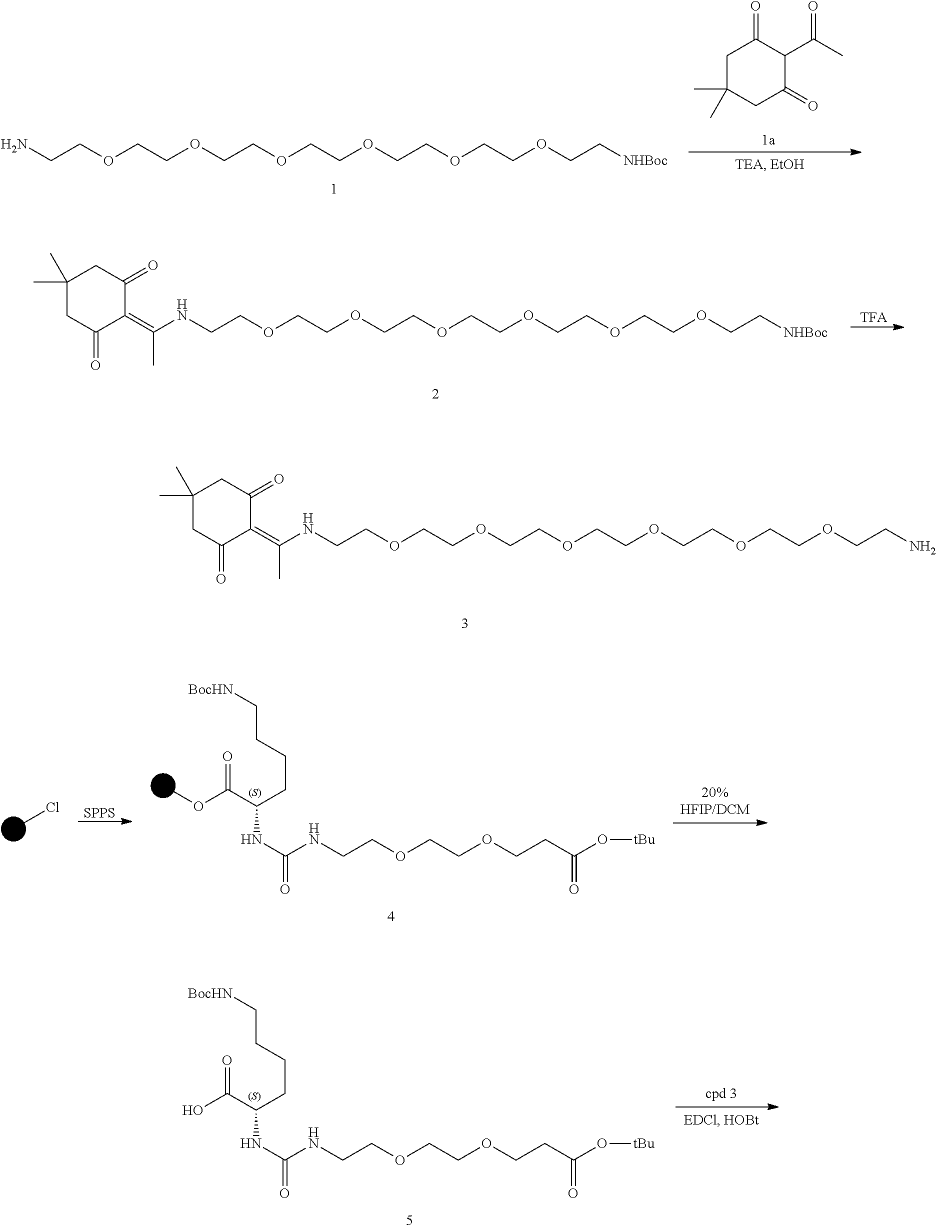

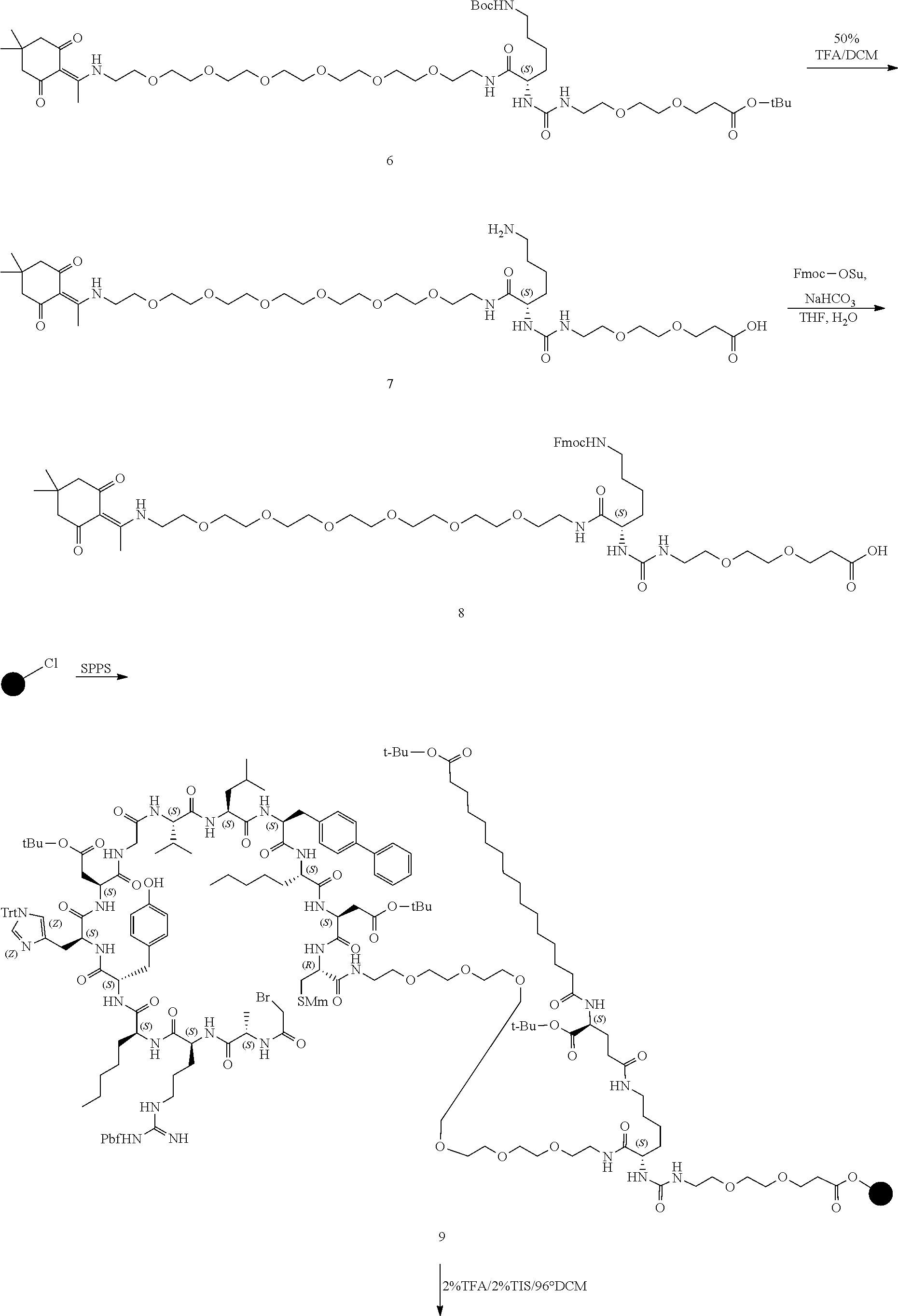

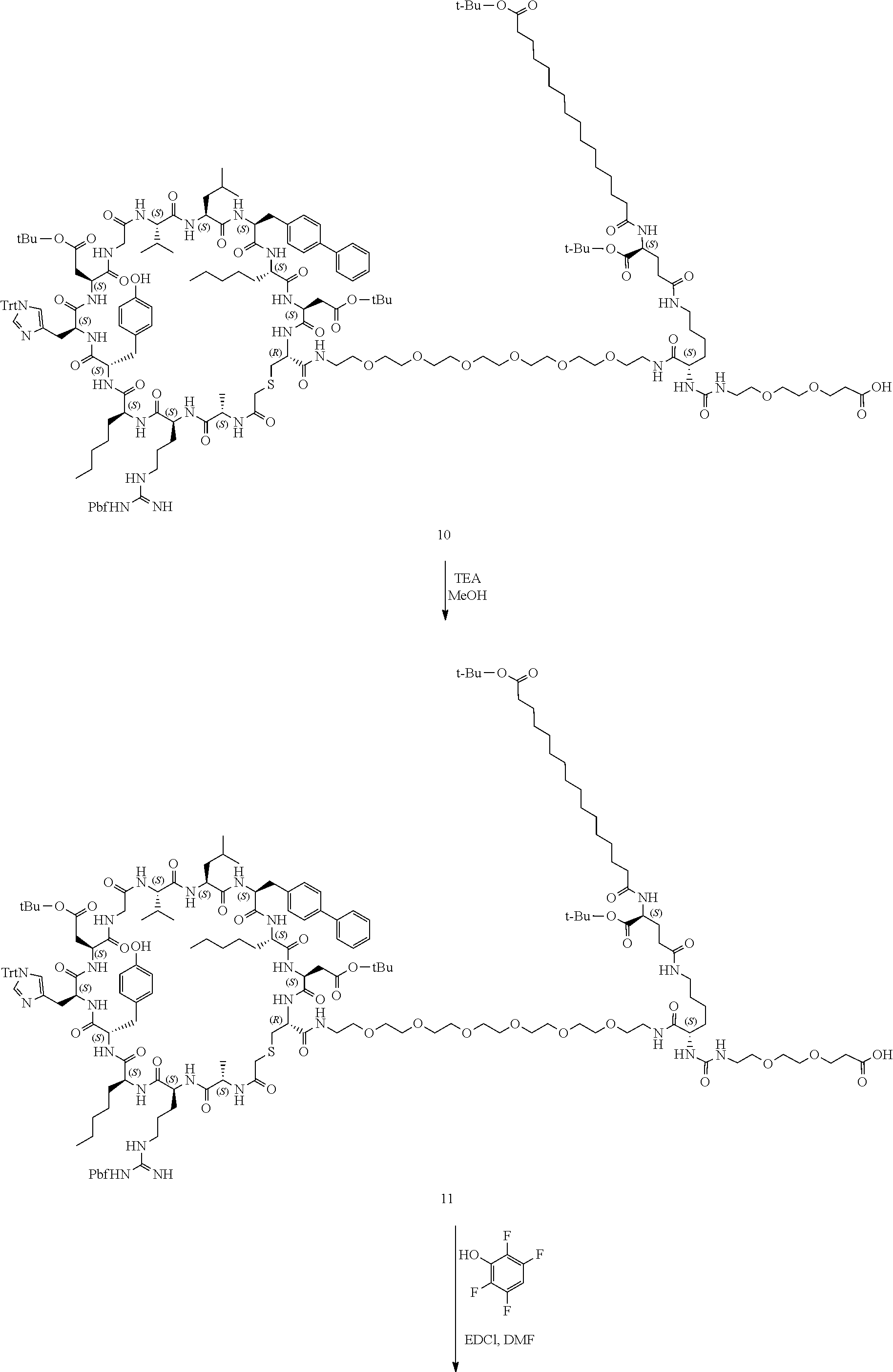

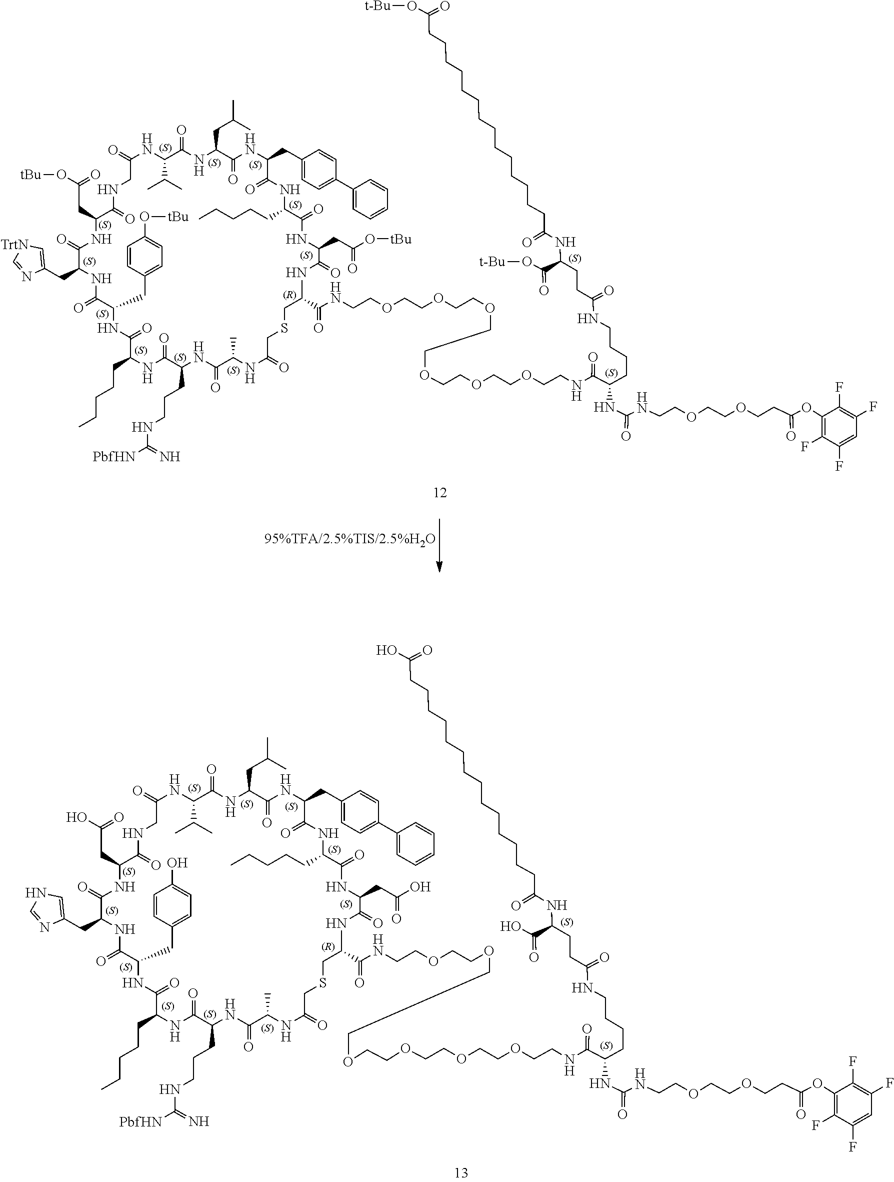

A mixture of compound 1a (1 g, 5.49 mmol, 1 eq), compound 1 (1.50 g, 3.53 mmol, 0.6 eq), TEA (1.11 g, 10.98 mmol, 1.53 mL, 2 eq) in EtOH (20 mL) was stirred at 90° C. for 16 hr. The solvent was removed under reduced pressure. The residue was diluted with DCM (100 mL), washed with 1 M HCl (20 mL), dried over anhydrous Na₂SO₄, concentrated under reduced pressure to get compound 2 (2.5 g, 4.25 mmol, 77.3% yield) as a brown oil.

Compound 2 (2.5 g, 4.25 mmol, 1 eq) in TFA (10 mL) and DCM (10 mL) was stirred at 15° C. for 0.5 hr. The solvent was removed under reduced pressure. The residue was purified by FLASH C18 (ISCO®; 120 g SepaFlash® C18 Flash Column, Eluent of 0-90% MeCN/H₂O ethergradient @ 75 mL/min) directly to get compound 3 (1.8 g, 2.99 mmol, 70.3% yield, TFA) as a brown oil.

Exemplary Peptide Synthesis. Peptide were synthesized using standard Fmoc chemistry, for example:

1) Resin preparation: To the vessel containing CTC Resin (2.0 mmol, 2.0 g, 1.0 mmol/g) and Fmoc-Lys(Boc)-OH (936 mg, 2.0 mmol, 1.00 eq) in DCM (20 mL) was added DIEA (4.00 eq) dropwise and mix for 2 hr with N₂ bubbling at 15° C. Then added MeOH (2 mL) and bubbled with N₂ for another 30 min. The resin was washed with DMF (40 mL)*5. Then 20% piperidine in DMF (40 mL) was added and the mixture was bubbled with N₂ for 30 mins at 15° C. Then the mixture was filtered to obtain the resin. The resin was washed with DMF (20 mL)*5 before proceeding to next step.
2) Coupling: A mixture of tert-butyl 3-(2-(2-aminoethoxy)ethoxy)propanoate (2.00 eq), CDI (2.0 eq) in DMF (5 mL) was stirred at 15° C. for 1 h. Then the resulting mixture and DMAP (0.4 eq) were added to the resin with N₂ bubbling at 15° C. for 72 hrs. The coupling reaction was monitored by ninhydrin test, and it showed colorless, indicating that the coupling was completed. The resin was then washed with DMF (20 mL)*5, MeOH (20 mL)*5 and then dried under vacuum.

| # | Materials | Coupling reagents |
|---|---|---|
| 1 | Fmoc-Lys(Boc)-OH (1.00 eq) | DIEA (4.00 eq) |
| 2 | tert-butyl 3-(2-(2-aminoethoxy)ethoxy)propanoate (2.00 eq) | CDI (2.00 eq), DMAP (0.4 eq) |

Peptide Cleavage and Purification:

1) Add cleavage buffer (20% HFIP/DCM, 40 mL) to the flask containing the sidechain protected peptide at room temperature and stirred for 30 mins twice.
2) Filter and collect the filtrate.
3) The combined filtrate was concentrated under reduced pressure.
4) The residue was purified by Flash C18 (neutral condition, H₂O/MeCN) to give compound 5 (500 mg, crude) as a colorless oil.

A mixture of compound 3 (519 mg, 1.06 mmol, 1.07 eq), compound 5 (0.5 g, 988 umol, 1.0 eq), DIEA (383 mg, 2.97 mmol, 516.76 uL, 3.0 eq), HBTU (375 mg, 988 umol, 1.0 eq) in DMF (20 mL) was stirred at 15° C. for 1 hr. The mixture was purified by FLASH C18 (ISCO®; 120 g SepaFlash® C18 Flash Column, Eluent of 0-90% MeCN/H₂O ethergradient @ 75 mL/min) directly to get compound 6 (0.6 g, 614.63 umol, 62.15% yield) as a colorless oil.

Compound 6 (0.6 g, 614 umol, 1 eq) was dissolved in TFA (6 mL) and DCM (6 mL), and stirred at 15° C. for 0.5 hr. The solvent was removed under reduced pressure. The residue was purified by FLASH C18 (ISCO®; 120 g SepaFlash® C18 Flash Column, Eluent of 0-90% MeCN/H2O ethergradient @ 75 mL/min) to get compound 7 (500 mg, 535 umol, 87.1% yield, TFA salt) as a colorless oil.

A mixture of compound 7 (500 mg, 535 umol, 1.0 eq, TFA), Fmoc-OSu (189 mg, 562 umol, 1.05 eq), DIEA (207 mg, 1.61 mmol, 279 uL, 3.0 eq) in DMF (5 mL) was stirred at 15° C. for 1 hr. The residue was purified by FLASH C18 (ISCO®; 120 g SepaFlash® C18 Flash Column, Eluent of 0-90% MeCN/H₂O ethergradient @ 75 mL/min) directly to get compound 8 (350 mg, 335 umol, 62.7% yield) as a white solid.

Exemplary Peptide Synthesis. Peptide were synthesized using standard Fmoc chemistry, for example:

1) Resin preparation: To the vessel containing CTC Resin (0.4 mmol, 0.4 g, 1.0 mmol/g, 1.2 eq) and compound 8 (350 mg, 335 umol, 1.0 eq) in DCM (3 mL) was added DIEA (4.00 eq) dropwise and mix for 2 hr with N₂ bubbling at 15° C. Then added MeOH (0.3 mL) and bubbled with N₂ for another 30 mins. The resin was washed with DMF (10 mL)*5. Then 20% piperidine in DMF (10 mL) was added and the mixture was bubbled with N₂ for 30 min at 15° C. Then the mixture was filtered to obtain the resin. The resin was washed with DMF (10 mL)*5 before proceeding to next step.
2) Coupling: A solution of Fmoc-Glu-OtBu (3.00 eq), HBTU (2.85 eq) in DMF (5 mL) was added to the resin with N₂ bubbling. Then DIEA (6.00 eq) was added to the mixture dropwise and bubbled with N₂ for 30 min at 15° C. The coupling reaction was monitored by ninhydrin test, if it showed colorless, the coupling was completed. If it showed blur or brownish red, the coupling was repeated and checked with ninhydrin test again till reaching completion. The resin was then washed with DMF (10 mL)*5.
3) De-protection of Fmoc: 20% piperidine in DMF (10 mL) was added to the resin and the mixture was bubbled with N₂ for 30 min at 15° C. The resin was then washed with DMF (10 mL)*5. The De-protection reaction was monitored by ninhydrin test, if it showed blue or other brownish red, the reaction was completed.
4) Repeat Step 2 for next amino acid: (No. 3, 16-(tert-butoxy)-16-oxohexadecanoic acid, in Table 2).
5) De-protection of Dde: 3% N₂H4.H₂O in DMF (10 mL) was added to the resin and the mixture was bubbled with N₂ for 30 min at 15° C. The resin was then washed with DMF (10 mL)*5. The De-protection reaction was monitored by ninhydrin test, and it showed blue or other brownish red, indicating that the reaction was completed.
6) Repeat Step 2 and 3 for all other amino acids: see below.
7) Coupling for the last position: A solution of 2-bromoacetic acid (4.00 eq) and DIC (4.00 eq) was added to resin and the mixture was bubbled with N₂ for 20 min. The coupling reaction was monitored by ninhydrin test, if it showed colorless, the coupling was completed. The resin was then washed with DMF (10 mL)*5, MeOH (10 mL)*5, and then dried under vacuum.

| # | Materials | Coupling reagents |
|---|---|---|
| 1 | compound 8 (1.0 eq) | DIEA (4.0 eq) |
| 2 | Fmoc-Glu-OtBu (3.0 eq) | HBTU (2.85 eq) and DIEA (6.0 eq) |
| 3 | 16-(tert-butoxy)-16-oxohexadecanoic acid, (2.0 eq) | HATU (1.90 eq) and DIEA (4.0 eq) |
| 4 | Fmoc-Cys(Mmt)-OH (2.0 eq) | HATU (1.90 eq) and DIEA (4.0 eq) |
| 5 | Fmoc-Asp(OtBu)—OH (3.0 eq) | HBTU (2.85 eq) and DIEA (6.0 eq) |
| 6 | Fmoc-HomNle-OH (2.0 eq) | HATU (1.90 eq) and DIEA (4.0 eq) |
| 7 | Fmoc-Bip-OH (2.0 eq) | HATU (1.90 eq) and DIEA (4.0 eq) |
| 8 | Fmoc-Leu-OH (3.0 eq) | HBTU (2.85 eq) and DIEA (6.0 eq) |
| 9 | Fmoc-Val-OH (3.0 eq) | HBTU (2.85 eq) and DIEA (6.0 eq) |
| 10 | Fmoc-Gly-OH (3.0 eq) | HBTU (2.85 eq) and DIEA (6.0 eq) |
| 11 | Fmoc-Asp(OtBu)—OH (3.0 eq) | HBTU (2.85 eq) and DIEA (6.0 eq) |
| 13 | Fmoc-His(Trt)-OH (3.0 eq) | HBTU (2.85 eq) and DIEA (6.0 eq) |
| 13 | Fmoc-Tyr(tBu)—OH (3.0 eq) | HBTU (2.85 eq) and DIEA (6.0 eq) |
| 14 | Fmoc-HomNle-OH (2.0 eq) | HATU (1.90 eq) and DIEA (4.0 eq) |
| 15 | Fmoc-Arg(Pbf)-OH (3.0 eq) | HBTU (2.85 eq) and DIEA (6.0 eq) |
| 16 | Fmoc-Ala-OH (3.0 eq) | HBTU (2.85 eq) and DIEA (6.0 eq) |
| 17 | 2-bromoacetic acid (4.0 eq) | DIC (2.0 eq) |

### Peptide Cleavage and Purification:

1) Add cleavage buffer (2% TFA/2% Tis/96% DCM, 40 mL) to the flask containing the side chain protected peptide at room temperature with N₂ bubbling for 20 min.
2) Filter and collect the filtrate. The clear solution is Compound 10 (335 umol) in cleavage buffer (400 mL), and is used in next step directly.

Compound 10 (335 umol, 400 mL, in cleavage buffer) was diluted with MeOH (400 mL), based with TEA to pH=8 under N₂ atmosphere, then stirred at 15° C. for 2 hr. The solvent was removed under reduced pressure, and the residue was triturated in 0.1 M HCl (cold, 100 mL). After filtration, the solid was washed with H₂O (50 mL), and then stirred in isopropyl ether (50 mL) for 10 min. Finally, the solid was dried under reduced pressure to get compound 11 (1.0 g, crude).

A mixture of compound 11 (650 mg, 186 umol, 1.0 eq), 2,3,5,6-tetrafluorophenol (155 mg, 934 umol, 5.0 eq), EDCI (107 mg, 560 umol, 3.0 eq) in DMF (6 mL) was stirred at 15° C. for 3 hr. The mixture was added to 0.5 M HCl (cold, 50 mL), after filtered, the solid was washed with H₂O (cold, 50 mL), isopropyl ether (50 mL), dried via lyophilization to get compound 12 (700 mg, crude) as a white solid.

Compound 12 (600 mg, 165 umol) was treated with deprotection cocktail containing TFA (15.40 g, 135 mmol, 10 mL), H₂O (250 mg, 13.88 mmol, 0.25 mL), and triisopropylsilane (192.75 mg, 1.22 mmol, 0.25 mL), and was stirred at 15° C. for 1 hr. The mixture was precipitated with cold isopropyl ether (50 mL) and centrifuged (3 min at 3000 rpm). Isopropyl ether wash two additional times (50 mL). Dry the crude peptide under vacuum 2 hrs. The solution was purified by prep-HPLC (TFA condition) directly to get compound 13 (25 mg, 8.60 umol, 5.2% yield) as a white solid.

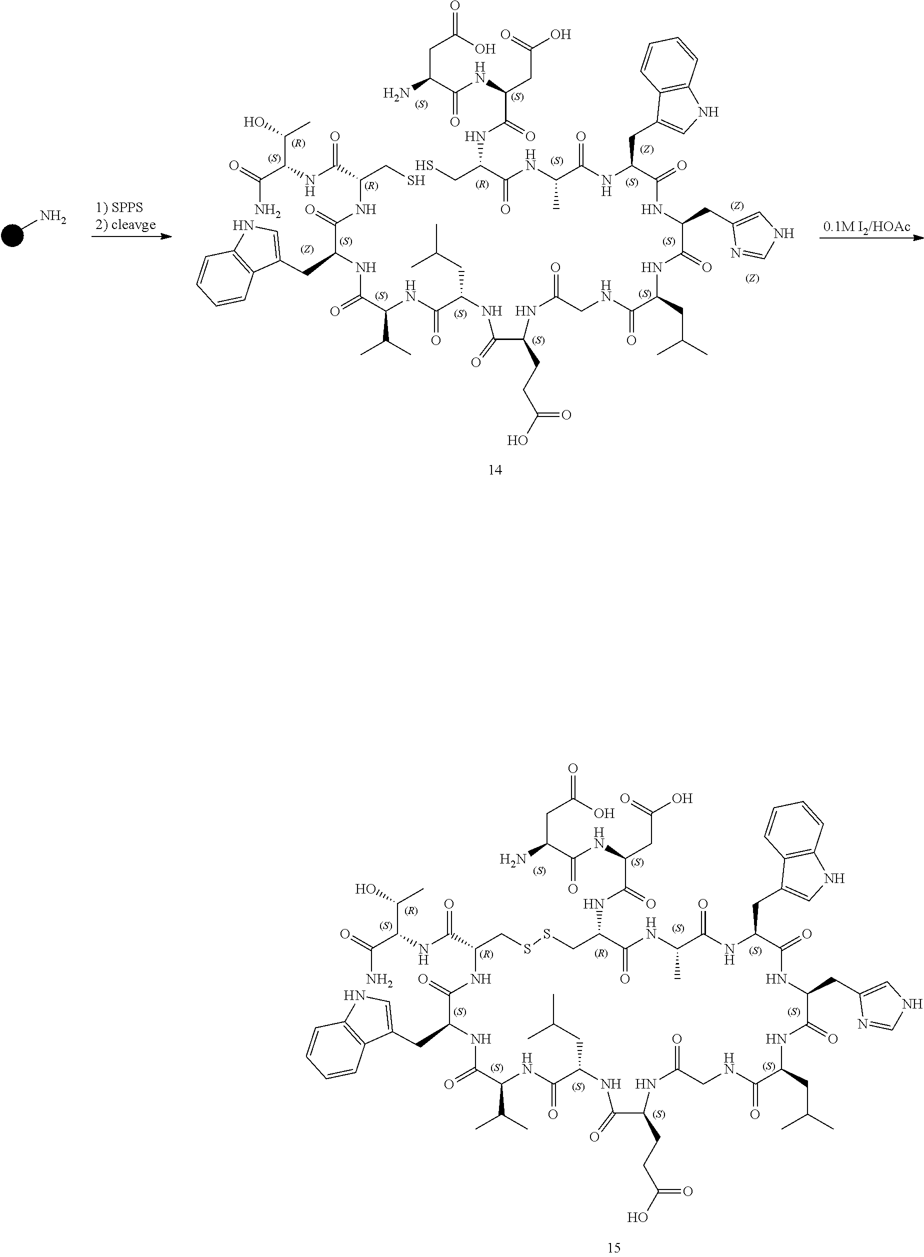

Peptide were synthesized using standard Fmoc chemistry, for example:

1) Resin preparation: To the vessel containing Rink Amide MBHA Resin (0.2 mmol, 0.65 g, 0.31 mmol/g) in DMF (5 mL) with N₂ bubbling at 15° C. for 30 min. Then 20% piperidine in DMF (10 mL) was added and the mixture was bubbled with N₂ for 30 min at 15° C. Then the mixture was filtered to obtain the resin. The resin was washed with DMF (10 mL)*5 before proceeding to next step.
2) Coupling: A solution of Fmoc-Thr(tBu)-OH (3.00 eq), HBTU (2.85 eq) in DMF (5 mL) was added to the resin with N₂ bubbling. Then DIEA (6.00 eq) was added to the mixture dropwise and bubbled with N₂ for 30 mins at 15° C. The coupling reaction was monitored by ninhydrin test, if it showed colorless, the coupling was completed. The resin was then washed with DMF (10 mL)*5.
3) De-protection: 20% piperidine in DMF (10 mL) was added to the resin and the mixture was bubbled with N₂ for 30 mins at 15° C. The resin was then washed with DMF (10 mL)*5. The De-protection reaction was monitored by ninhydrin test, if it showed blue or other brownish red, the reaction was completed.
4) Repeat Step 2 and 3 for all other amino acids: see below.
5) After the last position completed, the resin was washed with DMF (10 mL)*5, MeOH (10 mL)*5 and then dried under vacuum.

| # | Materials | Coupling reagents |
|---|---|---|
| 1 | Fmoc-Thr(tBu)—OH (3.00 eq) | HBTU (2.85 eq) and DIEA(6.00 eq) |
| 2 | Fmoc-Cys(Trt)-OH (3.00 eq) | HBTU (2.85 eq) and DIEA(6.00 eq) |
| 3 | Fmoc-Trp-OH (3.00 eq) | HBTU (2.85 eq) and DIEA(6.00 eq) |
| 4 | Fmoc-Val-OH (3.00 eq) | HBTU (2.85 eq) and DIEA(6.00 eq) |
| 5 | Fmoc-Leu-OH (3.00 eq) | HBTU (2.85 eq) and DIEA(6.00 eq) |
| 6 | Fmoc-Glu(OtBu)—OH (3.00 eq) | HBTU (2.85 eq) and DIEA(6.00 eq) |
| 7 | Fmoc-Gly-OH (3.00 eq) | HBTU (2.85 eq) and DIEA(6.00 eq) |
| 8 | Fmoc-Leu-OH (3.00 eq) | HBTU (2.85 eq) and DIEA(6.00 eq) |
| 9 | Fmoc-His(Trt)-OH (2.00 eq) | HBTU (2.85 eq) and DIEA(6.00 eq) |
| 10 | Fmoc-Trp-OH (3.00 eq) | HBTU (2.85 eq) and DIEA(6.00 eq) |
| 11 | Fmoc-Ala-OH (3.00 eq) | HBTU (2.85 eq) and DIEA(6.00 eq) |
| 12 | Fmoc-Cys(Trt)-OH (3.00 eq) | HBTU (2.85 eq) and DIEA(6.00 eq) |
| 13 | Fmoc-Asp(OtBu)—OH (3.00 eq) | HBTU (2.85 eq) and DIEA(6.00 eq) |
| 14 | Fmoc-Asp(OtBu)—OH (3.00 eq) | HBTU (2.85 eq) and DIEA(6.00 eq) |

### Peptide Cleavage, Cyclization and Purification:

1) Add cleavage buffer (95% TFA/2.5% TIS/2.5% H₂O, 10 mL) to the flask containing the sidechain protected peptide at room temperature and stirred for 1 hr.
2) Filter and collect the filtrate.
3) The peptide is precipitated with cold isopropyl ether (100 mL) and centrifuged (3 mins at 3000 rpm).
4) Isopropyl ether washes two additional times, and dry the crude peptide under vacuum for 2 hr to get compound 14 (0.2 mmol, crude).
5) Compound 14 was dissolve in MeCN/H₂O (1:1, 200 mL), and then added 0.1 M I₂/HOAc dropwise until the light yellow persisted. After 10 min, the mixture was quenched with 0.1 M Na₂S₂O₃ dropwise until the light yellow disappeared. The mixture was dried via lyophilization, and the residue was purified by prep-HPLC (TFA, condition) directly to get compound 15 (120 mg, 90% purity).

A mixture of compound 11 (25 mg, 8.7 umol, 1.0 eq), compound 15 (17.4 mg, 10.5 umol, 1.2 eq), DIEA (11.3 mg, 87.7 umol, 15.3 uL, 10 eq) in DMF (0.2 mL) was stirred at 15° C. for 1 hr. The solution was purified by prep-HPLC (TFA condition) directly to get I-32 (8.9 mg, 1.89 umol, 21.5% yield, 92% purity) as a white solid. LCMS: 1443.4 (+ESI Scan; major peak). Purification condition:

Separation condition

| Sample Preparation | Dissolve in MeCN/H₂O |
|---|---|
| Instrument | Gilson GX-215 |
| Mobile Phase | A: H₂O (0.075% TFA in H₂O) |
| | B: MeCN |
| Gradient | 30-60%-60 min. Retention time: 40 min |
| Column | Luna25*200 mm, C18 10 um, 110 A + |
| | Gemin150*30 mm, C18 5 um, 110 A° |
| Flow Rate | 20 mL/min |
| Wavelength | 220/254 nm |
| Oven Tem. | Room temperature |

Exemplary Preparation of I-38.

Exemplary Peptide Synthesis. Peptide were synthesized using standard Fmoc chemistry, for example:

1) Resin preparation: To the vessel containing Rink Amide MBHA Resin (0.2 mmol, 0.65 g, 0.31 mmol/g) in DMF (5 mL) with N₂ bubbling at 15° C. for 30 min. Then 20% piperidine in DMF (10 mL) was added and the mixture was bubbled with N₂ for 30 mins at 15° C. Then the mixture was filtered to obtain the resin. The resin was washed with DMF (10 mL)*5 before proceeding to next step.
2) Coupling: A solution of Fmoc-Thr(tBu)-OH (3.00 eq), HBTU (2.85 eq) in DMF (5 mL) was added to the resin with N₂ bubbling. Then DIEA (6.00 eq) was added to the mixture dropwise and bubbled with N₂ for 30 mins at 15° C. The coupling reaction was monitored by ninhydrin test, if it showed colorless, the coupling was completed. The resin was then washed with DMF (10 mL)*5.
3) De-protection: 20% piperidine in DMF (10 mL) was added to the resin and the mixture was bubbled with N₂ for 30 mins at 15° C. The resin was then washed with DMF (10 mL)*5. The De-protection reaction was monitored by ninhydrin test, if it showed blue or other brownish red, the reaction was completed.
4) Repeat Step 2 and 3 for all other amino acids: see below.
5) After the last position completed, the resin was washed with DMF (10 mL)*5, MeOH (10 mL)*5 and then dried under vacuum.
Note: 3% N₂H₄.H₂O/DMF was used for Dde de-protection.

| # | Materials | Coupling reagents |
|---|---|---|
| 1 | Fmoc-Thr(tBu)—OH (3.00 eq) | HBTU (2.85 eq) and DIEA(6.00 eq) |
| 2 | Fmoc-Cys(Trt)-OH (3.00 eq) | HBTU (2.85 eq) and DIEA(6.00 eq) |
| 3 | Fmoc-Trp-OH (3.00 eq) | HBTU (2.85 eq) and DIEA(6.00 eq) |
| 4 | Fmoc-Val-OH (3.00 eq) | HBTU (2.85 eq) and DIEA(6.00 eq) |
| 5 | Fmoc-Leu-OH (3.00 eq) | HBTU (2.85 eq) and DIEA(6.00 eq) |
| 6 | Fmoc-Glu(OtBu)—OH (3.00 eq) | HBTU (2.85 eq) and DIEA(6.00 eq) |
| 7 | Fmoc-Gly-OH (3.00 eq) | HBTU (2.85 eq) and DIEA(6.00 eq) |
| 8 | Fmoc-Leu-OH (3.00 eq) | HBTU (2.85 eq) and DIEA(6.00 eq) |
| 9 | Fmoc-His(Trt)-OH (2.00 eq) | HBTU (2.85 eq) and DIEA(6.00 eq) |
| 10 | Fmoc-Trp-OH (3.00 eq) | HBTU (2.85 eq) and DIEA(6.00 eq) |
| 11 | Fmoc-Ala-OH (3.00 eq) | HBTU (2.85 eq) and DIEA(6.00 eq) |
| 12 | Fmoc-Cys(Trt)-OH (3.00 eq) | HBTU (2.85 eq) and DIEA(6.00 eq) |
| 13 | Fmoc-Asp(OtBu)—OH (3.00 eq) | HBTU (2.85 eq) and DIEA(6.00 eq) |
| 14 | 6-(4-(((9H-fluoren-9-yl)methoxy)carbonyl)piperazin-1- | HATU (1.9 eq) and DIEA(4.00 eq) |
| | yl)nicotinic acid (2.00 eq) | |
| 15 | Fmoc-D-Lys(Dde)-OH (2.00 eq) | HATU (1.9 eq) and DIEA(4.00 eq) |
| 16 | 6-(4-(((9H-fluoren-9-yl)methoxy)carbonyl)piperazin-1- | HATU (1.9 eq) and DIEA(4.00 eq) |
| | yl)nicotinic acid (2.00 eq) | |
| 17 | 6-(4-(((9H-fluoren-9-yl)methoxy)carbonyl)piperazin-1- | HATU (1.9 eq) and DIEA(4.00 eq) |
| | yl)nicotinic acid (2.00 eq) | |
| 18 | Boc₂O (6 eq) | DIEA(1200 eq) |
| 19 | Fmoc-Glu-OtBu (2.00 eq) | HATU (1.9 eq) and DIEA(4.00 eq) |
| 20 | 16-(tert-butoxy)-16-oxohexadecanoic acid (2.00 eq) | HATU (1.9 eq) and DIEA(4.00 eq) |

### Peptide Cleavage and Purification:

1) Add cleavage buffer (95% TFA/2.5% TIS/2.5% H₂O, 10 mL) to the flask containing the sidechain protected peptide at room temperature and stirred for 1 hr.
2) Filter and collect the filtrate.
3) The peptide is precipitated with cold isopropyl ether (100 mL) and centrifuged (3 min at 3000 rpm).
4) Isopropyl ether washes two additional times, and dry the crude peptide under vacuum for 2 hrs.
5) To a mixture of compound 1 in MeCN/H₂O (200 mL) was added 0.1 M I₂/HOAc dropwise until the light yellow persisted, then the mixture was quenched with 0.1 M Na₂S₂O₃ dropwise until the light yellow disappeared. The mixture was dried via lyophilization. The residue was purified by prep-HPLC (TFA condition) directly to get compound 2 (125 mg) as a white solid.

Another exemplary Peptide Synthesis. Peptide were synthesized using standard Fmoc chemistry, for example:

1) Resin preparation: To the vessel containing CTC Resin (0.5 mmol, 0.5 g, 1.0 mmol/g) and 5-((((9H-fluoren-9-yl)methoxy)carbonyl)amino)pentanoic acid (0.17 g, 0.5 mmol, 1.00 eq) in DCM (5 mL) was added DIEA (4.00 eq) dropwise and mix for 2 hr with N₂ bubbling at 15° C. Then added MeOH (0.5 mL) and bubbled with N₂ for another 30 min. The resin was washed with DMF (20 mL)*5. Then 20% piperidine in DMF (10 mL) was added and the mixture was bubbled with N₂ for 30 mins at 15° C. Then the mixture was filtered to obtain the resin. The resin was washed with DMF (10 mL)*5 before proceeding to next step.
2) Coupling: A solution of Fmoc-Cys(Mmt)-OH (2.00 eq), HBTU (1.90 eq) in DMF (5 mL) was added to the resin with N₂ bubbling. Then DIEA (4.00 eq) was added to the mixture dropwise and bubbled with N₂ for 30 min at 15° C. The coupling reaction was monitored by ninhydrin test, if it showed colorless, the coupling was complete. The resin was then washed with DMF (10 mL)*5.
3) De-protection: 20% piperidine in DMF (10 mL) was added to the resin and the mixture was bubbled with N₂ for 30 mins at 15° C. The resin was then washed with DMF (10 mL)*5. The De-protection reaction was monitored by ninhydrin test, if it showed blue or other brownish red, the reaction was complete.
4) Repeat Step 2 and 3 for all other amino acids: see below.
5) Coupling for the last position: A solution of 2-bromoacetic acid (4.00 eq) and DIC (4.00 eq) was added to resin and the mixture was bubbled with N₂ for 20 mins. The coupling reaction was monitored by ninhydrin test, if it showed colorless, the coupling was completed. The resin was then washed with DMF (10 mL)*5, MeOH (10 mL)*5, and then dried under vacuum.

| # | Materials | Coupling reagents |
|---|---|---|
| 1 | 5-((((9H-fluoren-9- | DIEA (4.00 eq) |
| | yl)methoxy)carbonyl)amino)pentanoic acid (1.00 eq) | |
| 2 | Fmoc-Cys(Mmt)-OH (2.00 eq) | HATU (1.90 eq) and DIEA(4.00 eq) |
| 3 | Fmoc-Asp(OtBu)—OH (3.00 eq) | HBTU (2.85 eq) and DIEA(6.00 eq) |
| 4 | (S)-2-((((9H-fluoren-9- | HATU (1.35 eq) and DIEA(3.00 eq) |
| | yl)methoxy)carbonyl)amino)heptanoic acid (1.50 eq) | |
| 5 | (S)-2-((((9H-fluoren-9-yl)methoxy)carbonyl)amino)-3- | HATU (1.35 eq) and DIEA(3.00 eq) |
| | ([1,1′-biphenyl]-4-yl)propanoic acid (1.50 eq) | |
| 6 | Fmoc-Leu-OH (3.00 eq) | HBTU (2.85 eq) and DIEA(6.00 eq) |
| 7 | Fmoc-Val-OH (3.00 eq) | HBTU (2.85 eq) and DIEA(6.00 eq) |
| 8 | Fmoc-Gly-OH (3.00 eq) | HBTU (2.85 eq) and DIEA(6.00 eq) |
| 9 | Fmoc-Asp(OtBu)—OH (3.00 eq) | HBTU (2.85 eq) and DIEA(6.00 eq) |
| 10 | Fmoc-His(Trt)-OH (3.00 eq) | HBTU (2.85 eq) and DIEA(6.00 eq) |
| 11 | Fmoc-Tyr(tBu)—OH (1.50 eq) | HBTU (2.85 eq) and DIEA(6.00 eq) |
| 12 | (S)-2-((((9H-fluoren-9- | HATU (1.35 eq) and DIEA(3.00 eq) |
| | yl)methoxy)carbonyl)amino)heptanoic acid (1.50 eq) | |
| 13 | Fmoc-Arg(Pbf)-OH (3.00 eq) | HBTU (2.85 eq) and DIEA(6.00 eq) |
| 14 | Fmoc-Ala-OH (3.00 eq) | HBTU (2.85 eq) and DIEA(6.00 eq) |
| 15 | 2-Bromoacetic acid (4.00 eq) | DIC (4.00 eq) |

### Peptide Cleavage and Purification:

3) Add cleavage buffer (2% TFA/2% Tis/96% DCM, 50 mL) to the flask containing the side chain protected peptide at room temperature with N₂ bubbling for 20 mins.
4) Filter and collect the filtrate. The clear solution is compound 3 (0.5 mmol) in cleavage buffer (50 mL), and is used in next step directly.

Compound 3 (0.5 mmol, 50 mL, in cleavage buffer) was diluted with MeOH (500 mL), based with TEA to pH=8 under N₂ atmosphere, then stirred at 15° C. for 2 hr. The solvent was removed under reduced pressure. The residue was triturated in 0.1 M HCl (cold, 50 mL), after filtration, the solid was washed with H₂O (50 mL), and then stirred in isopropyl ether (50 mL) for 10 min. Finally, the solid was dried under reduced pressure to get compound 4 (1.00 g, crude).

A mixture of compound 4 (1.00 g, 0.41 mmol, 1.00 eq), compound 4a (340 mg, 2.06 mmol, 5.00 eq), EDCI (236 mg, 1.23 mmol, 3.00 eq) in DMF (6 mL) was stirred at 15° C. for 3 hr. The mixture was added to 0.5 M HCl (cold, 60 mL), and there appeared lots of white solid. After filtration, the solid was washed with H₂O (50 mL), dried via lyophilization to get compound 5 (1.1 g, crude) as a white solid.

Compound 5 (1.1 g, crude) was treated with a mixture of solvent containing TFA (22.8 g, 200 mmol, 15 mL), H₂O (0.38 g, 20.8 mmol, 0.38 mL) and triisopropylsilane (0.29 g, 1.83 mmol, 0.38 mL), and was stirred at 15° C. for 1 hr. The mixture was precipitated with cold isopropyl ether (100 mL) and centrifuged (3 min at 3000 rpm). Cold isopropyl ether washed the precipitate two additional times (50 mL). Dry the crude peptide under vacuum 2 hr. The residue was purified by FLASH C18 (ISCO®; 120 g SepaFlash® C18 Flash Column, Eluent of 0-90% MeCN/H₂O gradient @ 75 mL/min) directly to get compound 6 (90 mg, 47.0 umol, 11.0% yield, 90.0% purity) as a white solid.

A mixture of compound 6 (40.0 mg, 20.9 umol, 1.00 eq), compound 2 (43.8 mg, 16.73 umol, 0.80 eq), DIEA (21.6 mg, 167 umol, 29.1 uL, 8.00 eq) in DMF (0.5 mL) was stirred at 15° C. for 1 hr. The solution was purified by prep-HPLC (acid condition, TFA) directly to get compound I-38 (Q1: 46.3 mg, 10.2 umol, 96.3% purity) and compound I-38 (Q2: 20.9 mg, 4.6 umol, 85.4% purity) as a white solid. LCMS: 874.9 (+ESI Scan; major peak; also 1457.6, 1093.3, 729.5, etc.).

Purification Conditions:

Purification condition

| Sample Preparation | Dissolve in MeCN/H₂O |
|---|---|
| Instrument | Gilson GX-215 |
| Mobile Phase | A: H₂O (0.075% TFA in H₂O) |
| | B: MeCN |
| Gradient | 30-60%-60 min. Retention time: 40 min |
| Column | Luna25*200 mm, C18 10 um, 110 A + |
| | Gemin150*30 mm, C18 5 um, 110 Å |
| Flow Rate | 20 mL/min |
| Wavelength | 220/254 nm |
| Oven Tem. | Room temperature |

Exemplary Preparation of I-39.

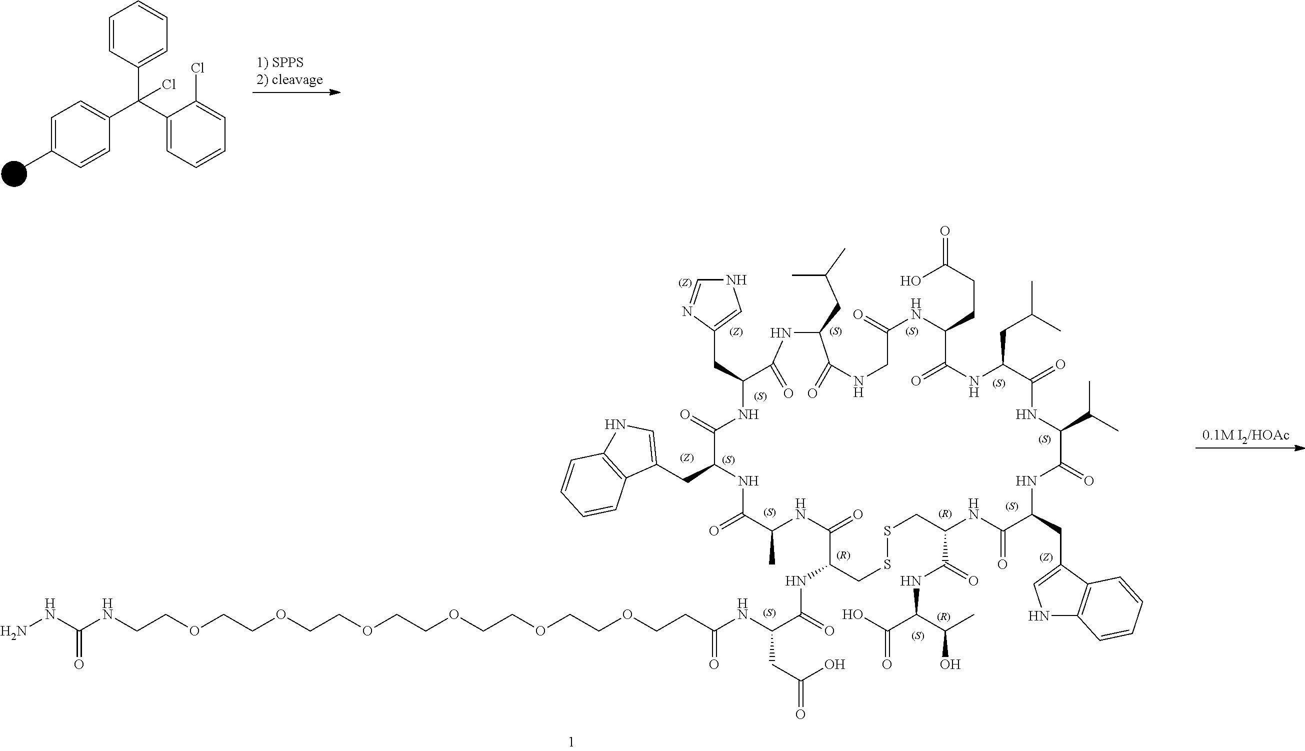

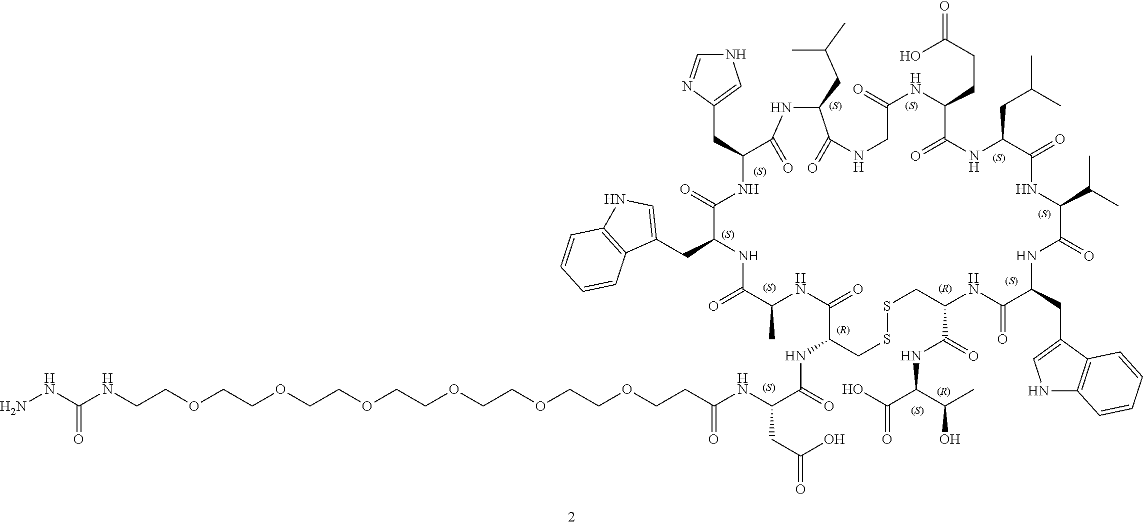

Exemplary Peptide Synthesis. Peptide were synthesized using standard Fmoc chemistry, for example:

1) Resin preparation: To the vessel containing CTC Resin (0.5 mmol, 0.50 g, 1.0 mmol/g) and Fmoc-Thr(tBu)-OH (0.198 g, 0.50 mmol, 1.00 eq) in DCM (5 mL) was added DIEA (4.00 eq) dropwise and mix for 2 hrs with N₂ bubbling at 15° C. Then added MeOH (0.5 mL) and bubbled with N₂ for another 30 mins. The resin was washed with DMF (10 mL)*5. Then 20% piperidine in DMF (10 mL) was added and the mixture was bubbled with N₂ for 30 mins at 15° C. Then the mixture was filtered to obtain the resin. The resin was washed with DMF (10 mL)*5 before proceeding to next step.
2) Coupling: A solution of Fmoc-Cys(Trt)-OH (3.00 eq), HBTU (2.85 eq) in DMF (5 mL) was added to the resin with N₂ bubbling. Then DIEA (6.00 eq) was added to the mixture dropwise and bubbled with N₂ for 30 mins at 15° C. The coupling reaction was monitored by ninhydrin test, if it showed colorless, the coupling was completed. If it showed blue or brownish red, the coupling was repeated and checked with ninhydrin test again till reaching completion. The resin was then washed with DMF (10 mL)*5.
3) De-protection: 20% piperidine in DMF (10 mL) was added to the resin and the mixture was bubbled with N₂ for 30 mins at 15° C. The resin was then washed with DMF (10 mL)*5. The De-protection reaction was monitored by ninhydrin test, if it showed blue or other brownish red, the reaction was completed.
4) Repeat Step 2 and 3 for all other amino acids: see below.
5) Urea at the last position: A mixture of Boc-NH2-NH2 (6.00 eq), CDI (6.00 eq) and TEA (6.00 eq) in DMF (5 mL) was stirred at 15° C. for 1 h. Then the mixture and DMAP (6.00 eq) was added to the resin with N2 bubbling for 48 hrs. The reaction was monitored by ninhydrin test, if it showed colorless, the coupling was completed. The resin was then washed with DMF (10 mL)*5, MeOH (10 mL)*5 and then dried under vacuum.

| # | Materials | Coupling reagents |
|---|---|---|
| 1 | Fmoc-Thr(tBu)—OH (1.00 eq) | DIEA (4.00 eq) |
| 2 | Fmoc-Cys(Trt)-OH (3.00 eq) | HBTU (2.85 eq) and DIEA(6.00 eq) |
| 3 | Fmoc-Trp-OH (3.00 eq) | HBTU (2.85 eq) and DIEA(6.00 eq) |
| 4 | Fmoc-Val-OH (3.00 eq) | HBTU (2.85 eq) and DIEA(6.00 eq) |
| 5 | Fmoc-Leu-OH (3.00 eq) | HBTU (2.85 eq) and DIEA(6.00 eq) |
| 6 | Fmoc-Glu(OtBu)—OH (3.00 eq) | HBTU (2.85 eq) and DIEA(6.00 eq) |
| 7 | Fmoc-Gly-OH (3.00 eq) | HBTU (2.85 eq) and DIEA(6.00 eq) |
| 8 | Fmoc-Leu-OH (3.00 eq) | HBTU (2.85 eq) and DIEA(6.00 eq) |
| 9 | Fmoc-His(Trt)-OH (2.00 eq) | HBTU (2.85 eq) and DIEA(6.00 eq) |
| 10 | Fmoc-Trp-OH (3.00 eq) | HBTU (2.85 eq) and DIEA(6.00 eq) |
| 11 | Fmoc-Ala-OH (3.00 eq) | HBTU (2.85 eq) and DIEA(6.00 eq) |
| 12 | Fmoc-Cys(Trt)-OH (3.00 eq) | HBTU (2.85 eq) and DIEA(6.00 eq) |
| 13 | Fmoc-Asp(OtBu)—OH (3.00 eq) | HBTU (2.85 eq) and DIEA(6.00 eq) |
| 14 | Fmoc-PEG₆-CH₂CH₂COOH (2.00 eq) | HATU (1.90 eq) and DIEA(4.00 eq) |
| 15 | Boc-NH—NH2 (6.00 eq) | CDI (6.00 eq), TEA (6.00 eq), DMAP (6.00 eq) |

### Peptide Cleavage, Cyclization and Purification:

1) Add cleavage buffer (95% TFA/2.5% TIS/2.5% H₂O, 10 mL) to the flask containing the sidechain protected peptide at room temperature and stirred for 1 hr.
2) Filter and collect the filtrate.
3) The peptide is precipitated with cold isopropyl ether (100 mL) and centrifuged (3 mins at 3000 rpm).
4) Isopropyl ether washes two additional times, and dry the crude peptide under vacuum for 2 hrs.
5) Compound 1 is dissolved in MeCN/H₂O (500 mL), and then added 0.1 M I₂/HOAc dropwise until the light yellow persisted. 10 min later, the mixture is quenched with 0.1 M Na₂S₂O₃ dropwise until the light yellow disappears. The mixture is then dried via lyophilization. Finally, the residue is purified by prep-HPLC (acid condition, TFA) directly to get compound 2 (65 mg, 90% purity).

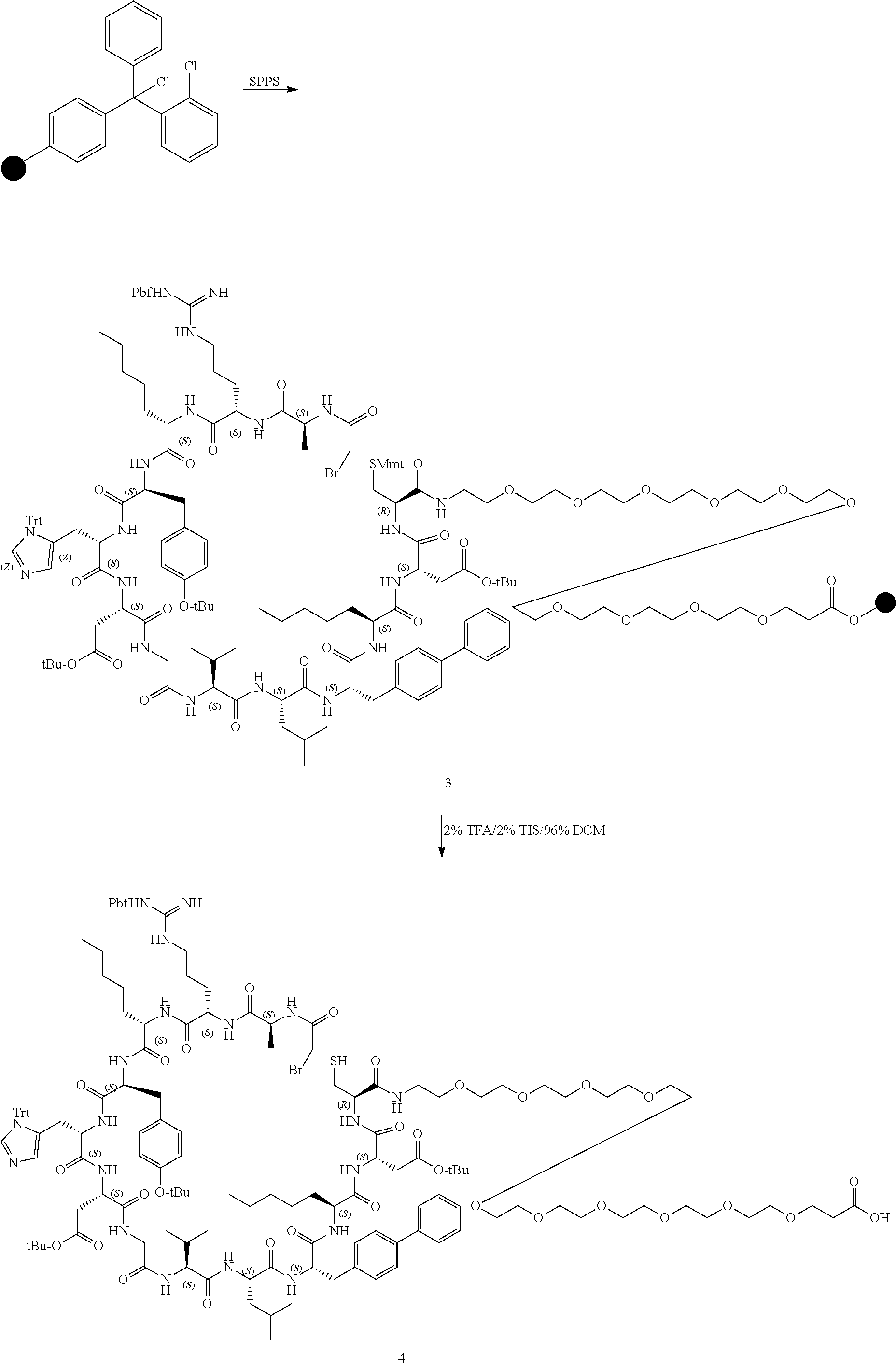

Another exemplary Peptide Synthesis. Peptide were synthesized using standard Fmoc chemistry, for example:

1) Resin preparation: To the vessel containing CTC Resin (1.0 mmol, 1.0 g, 1.0 mmol/g) and Fmoc-PEG₁₀-CH₂CH₂COOH (0.75 g, 1.0 mmol, 1.00 eq) in DCM (10 mL) was added DIEA (4.00 eq) dropwise and mix for 2 hrs with N₂ bubbling at 15° C. Then added MeOH (1.0 mL) and bubbled with N₂ for another 30 mins. The resin was washed with DMF (20 mL)*5. Then 20% piperidine in DMF (20 mL) was added and the mixture was bubbled with N₂ for 30 mins at 15° C. Then the mixture was filtered to obtain the resin. The resin was washed with DMF (20 mL)*5 before proceeding to next step.
2) Coupling: A solution of Fmoc-Cys(Mmt)-OH (2.00 eq), HBTU (1.90 eq) in DMF (5 mL) was added to the resin with N₂ bubbling. Then DIEA (4.00 eq) was added to the mixture dropwise and bubbled with N₂ for 30 mins at 15° C. The coupling reaction was monitored by ninhydrin test, if it showed colorless, the coupling was completed. If it showed blue or brownish red, the coupling was repeated and checked with ninhydrin test again till reaching completion. The resin was then washed with DMF (10 mL)*5.
3) De-protection: 20% piperidine in DMF (10 mL) was added to the resin and the mixture was bubbled with N₂ for 30 mins at 15° C. The resin was then washed with DMF (10 mL)*5. The De-protection reaction was monitored by ninhydrin test, if it showed blue or other brownish red, the reaction was completed.
4) Repeat Step 2 and 3 for all other amino acids: (2-14 in Table 2).
5) Coupling for the last position: A solution of 2-bromoacetic acid (4.00 eq) and DIC (4.00 eq) was added to resin and the mixture was bubbled with N₂ for 20 mins. The coupling reaction was monitored by ninhydrin test, if it showed colorless, the coupling was completed. The resin was then washed with DMF (10 mL)*5, MeOH (10 mL)*5, and then dried under vacuum.

| # | Materials | Coupling reagents |
|---|---|---|
| 1 | Fmoc-PEG₁₀-CH₂CH₂CH₂OH (1.00 eq) | DIEA (4.00 eq) |
| 2 | Fmoc-Cys(Mmt)-OH (2.00 eq) | HATU (1.90 eq) and DIEA(4.00 eq) |
| 3 | Fmoc-Asp(OtBu)—OH (3.00 eq) | HBTU (2.85 eq) and DIEA(6.00 eq) |
| 4 | (S)-2-((((9H-fluoren-9- | HATU (1.35 eq) and DIEA(3.00 eq) |
| | yl)methoxy)carbonyl)amino)heptanoic acid (1.50 eq) | |
| 5 | (S)-2-((((9H-fluoren-9-yl)methoxy)carbonyl)amino)-3- | HATU (1.35 eq) and DIEA(3.00 eq) |
| | ([1,1′-biphenyl]-4-yl)propanoic acid (1.50 eq) | |
| 6 | Fmoc-Leu-OH (3.00 eq) | HBTU (2.85 eq) and DIEA(6.00 eq) |
| 7 | Fmoc-Val-OH (3.00 eq) | HBTU (2.85 eq) and DIEA(6.00 eq) |
| 8 | Fmoc-Gly-OH (3.00 eq) | HBTU (2.85 eq) and DIEA(6.00 eq) |
| 9 | Fmoc-Asp(OtBu)—OH (3.00 eq) | HBTU (2.85 eq) and DIEA(6.00 eq) |
| 10 | Fmoc-His(Trt)-OH (3.00 eq) | HBTU (2.85 eq) and DIEA(6.00 eq) |
| 11 | Fmoc-Tyr(tBu)—OH (1.50 eq) | HBTU (2.85 eq) and DIEA(6.00 eq) |
| 12 | (S)-2-((((9H-fluoren-9- | HATU (1.35 eq) and DIEA(3.00 eq) |
| | yl)methoxy)carbonyl)amino)heptanoic acid (1.50 eq) | |
| 13 | Fmoc-Arg(Pbf)-OH (3.00 eq) | HBTU (2.85 eq) and DIEA(6.00 eq) |
| 14 | Fmoc-Ala-OH (3.00 eq) | HBTU (2.85 eq) and DIEA(6.00 eq) |
| 15 | 2-bromoacetic acid (4.00 eq) | DIC (4.00 eq) |

### Peptide Cleavage and Purification:

1) Add cleavage buffer (2% TFA/2% Tis/96% DCM, 100 mL) to the flask containing the side chain protected peptide at room temperature with N₂ bubbling for 20 mins.
2) Filter and collect the filtrate. The clear solution is Compound 4 (1.0 mmol) in cleavage buffer (100 mL), and is used in next step directly.

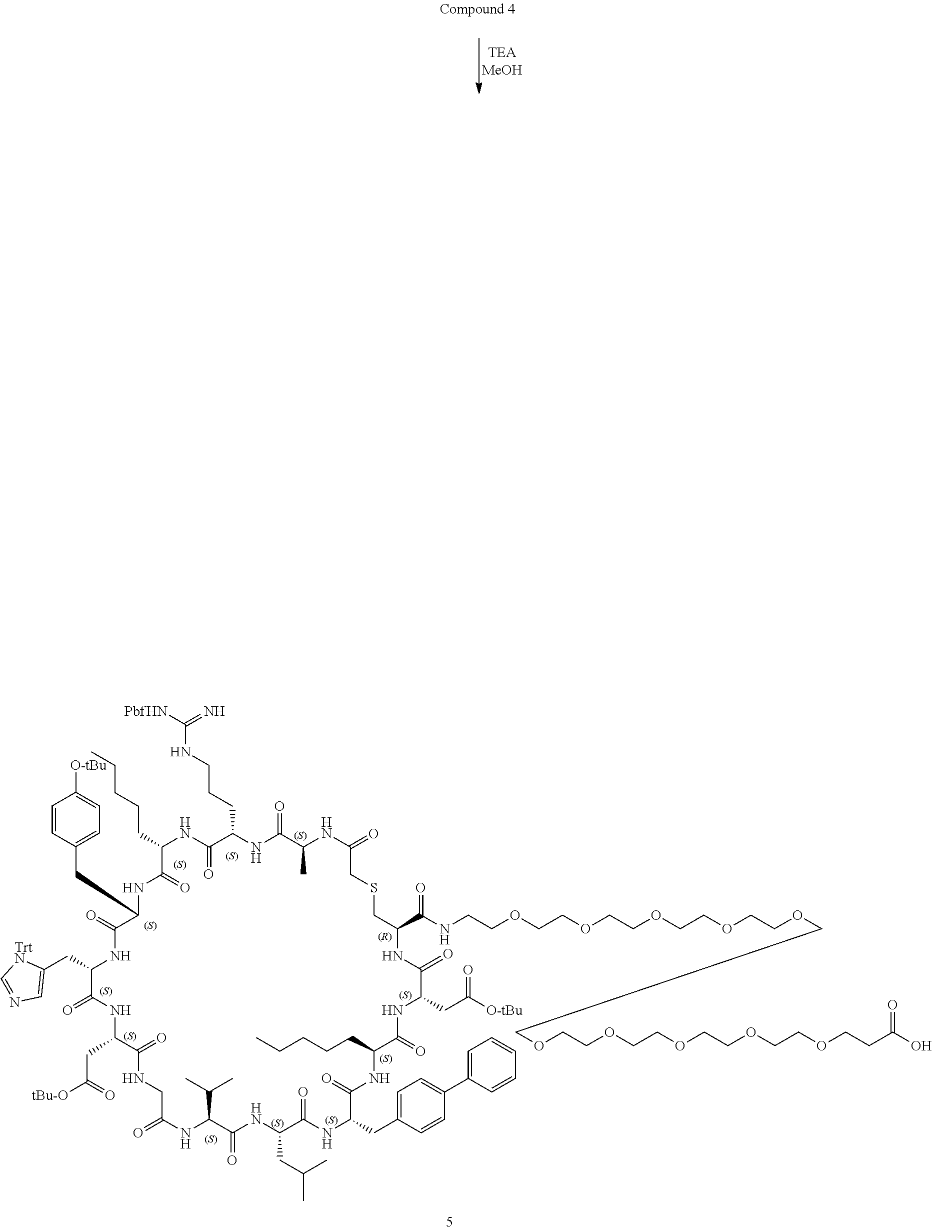

Compound 4 (1.0 mmol, 100 mL, in cleavage buffer) was diluted with MeOH (1000 mL), based with TEA to pH=8 under N₂ atmosphere, then stirred at 15° C. for 2 hrs. The solvent was removed under reduced pressure, and the residue was triturated in 0.1 M HCl (cold, 100 mL). After filtration, the solid was washed with H₂O (50 mL), and then stirred in isopropyl ether (50 mL) for 10 min. Finally, the solid was dried under reduced pressure to get compound 5 (1.80 g, crude).

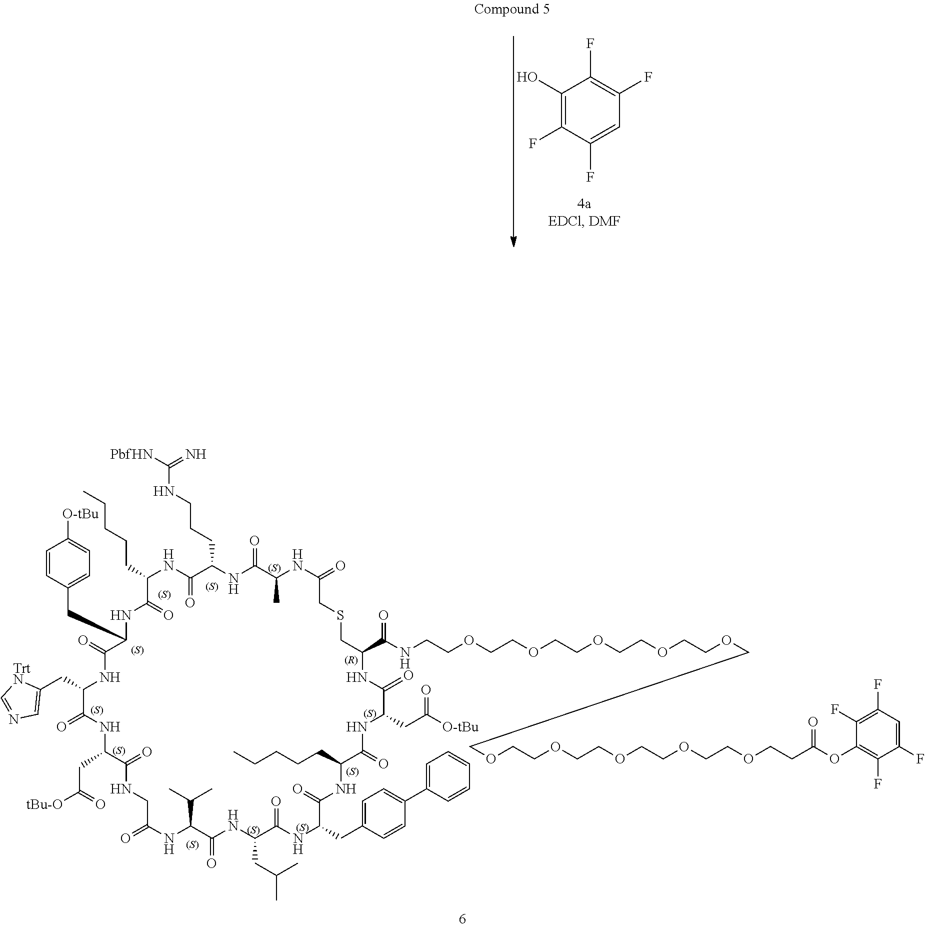

A mixture of compound 5 (1.80 g, 0.63 mmol, 1.00 eq), compound 4a (522 mg, 3.15 mmol, 5.00 eq), EDCI (361 mg, 1.89 mmol, 3.00 eq) in DMF (10 mL) was stirred at 15° C. for 3 hrs. The mixture was added to 0.5 M HCl (cold, 100 mL), there appeared lots of white solid. After filtration, the solid was washed with H₂O (20 mL), dried via lyophilization to get compound 6 (2.0 g, crude) as a white solid.

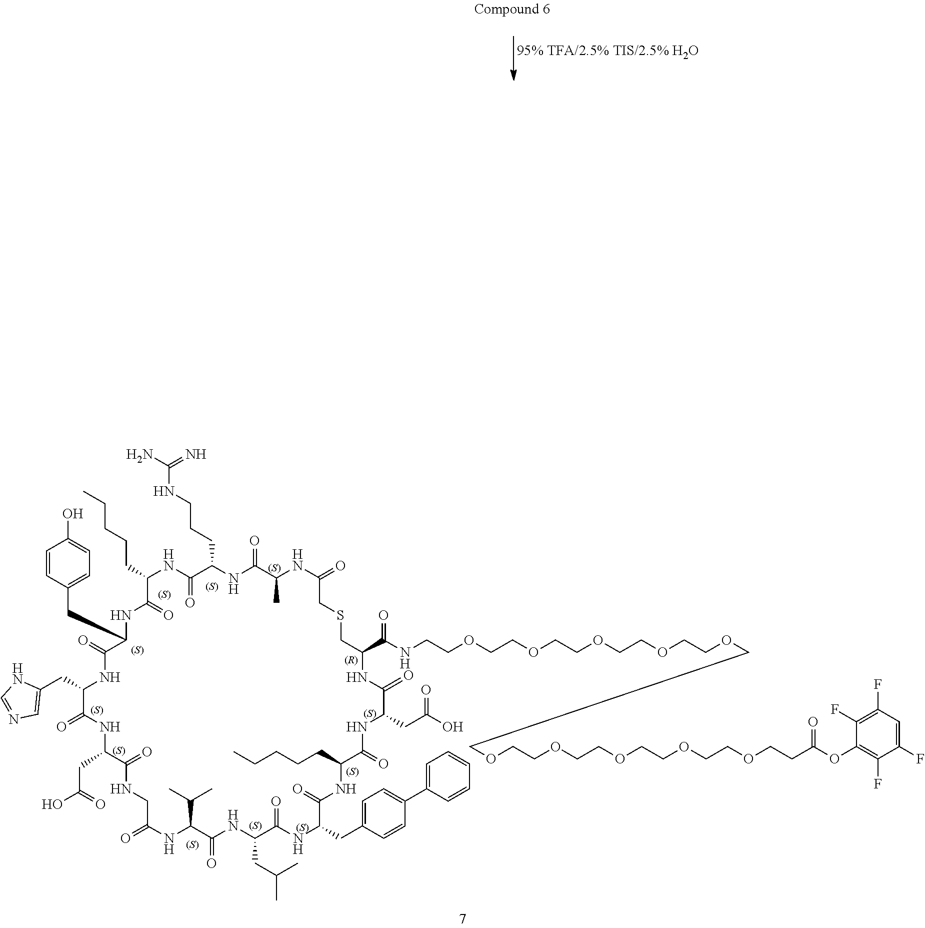

A mixture of compound 6 (2 g, crude) in TFA (45.6 g, 400 mmol, 30 mL), H₂O (0.75 g, 41.6 mmol, 0.75 mL), triisopropylsilane (0.58 g, 3.67 mmol, 0.75 mL) was stirred at 15° C. for 1 hr. The mixture was precipitated with cold isopropyl ether (100 mL) and centrifuged (3 mins at 3000 rpm). Isopropyl ether washed two additional times (50 mL). Dry the crude peptide under vacuum 2 hrs. The residue was purified by flash C18 (ISCO®; 120 g SepaFlash® C18 Flash Column, Eluent of 0-90% MeCN/H₂O gradient @ 75 mL/min) directly to get compound 7 (180 mg, 77.4 umol, 11.5% yield, 90.0% purity) as a white solid.

A mixture of compound 7 (47 mg, 20.22 umol, 1.00 eq), compound 2 (40 mg, 20.82 umol, 1.0 eq), DIEA (20.9 mg, 161.7 umol, 28.1 uL, 8 eq) in DMF (0.2 mL) was stirred at 15° C. for 30 min. The solution was purified by prep-HPLC (TFA condition) directly to obtain I-39 (Q1: 3.7 mg, 0.83 umol, 4.0% yield, 94% purity) and I-39 (Q2: 9.8 mg, 2.19 umol, 10.6% yield, 92% purity) as a white solid. LCMS: 1362.2 (+ESI Scan; major peak).

Purification Conditions:

Purification condition

| | |
|---|---|
| Sample Preparation | Dissolve in MeCN/H₂O |
| Instrument | Gilson GX-215 |
| Mobile Phase | A: H₂O (0.075% TFA in H₂O) |
| | B: MeCN |
| Gradient | 30-60%-60 min. Retention time: 40 min |
| Column | Luna25*200 mm, C18 10 um, 110A + Gemin150*30 mm, C18 5 um, 110A° |
| Flow Rate | 20 mL/min |
| Wavelength | 220/254 nm |
| Oven Tem. | Room temperature |

Exemplary Preparation of I-40.

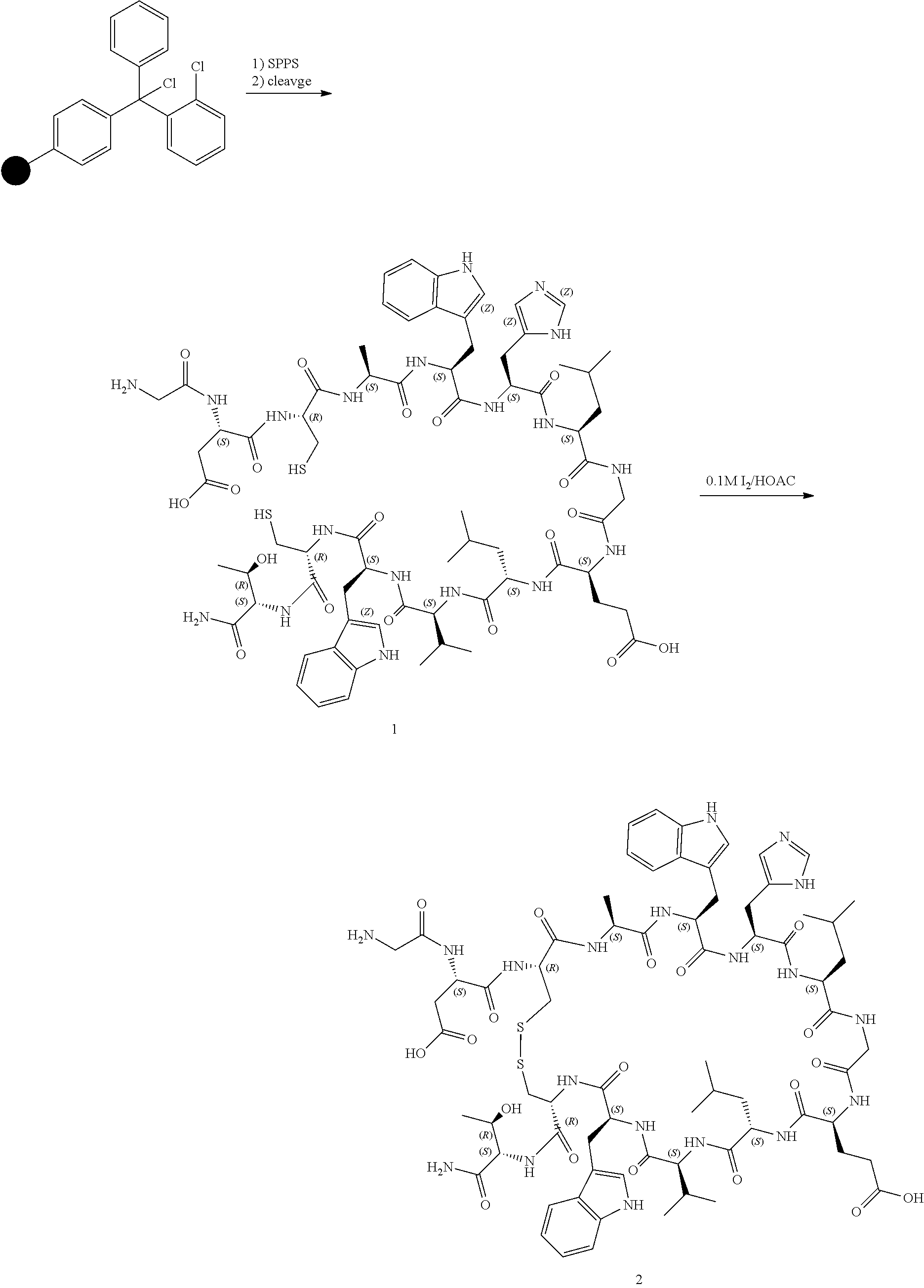

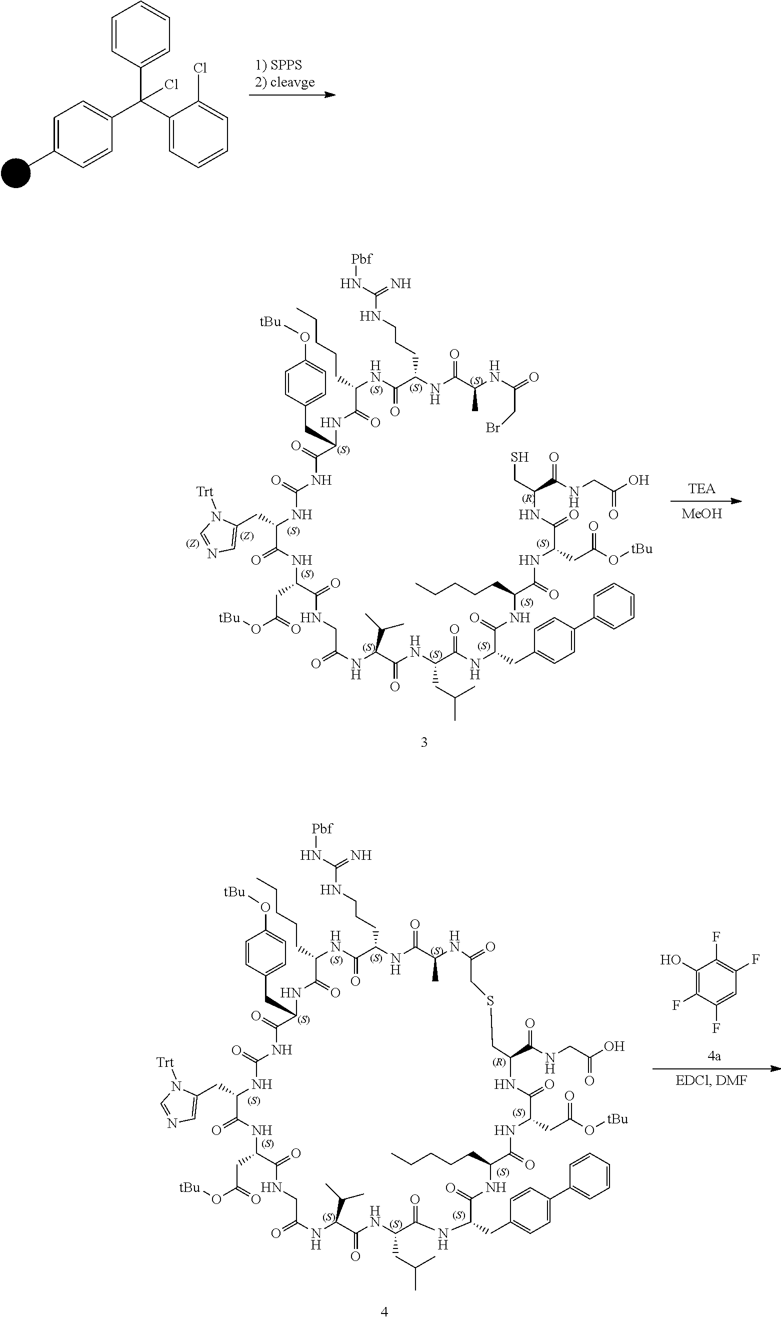

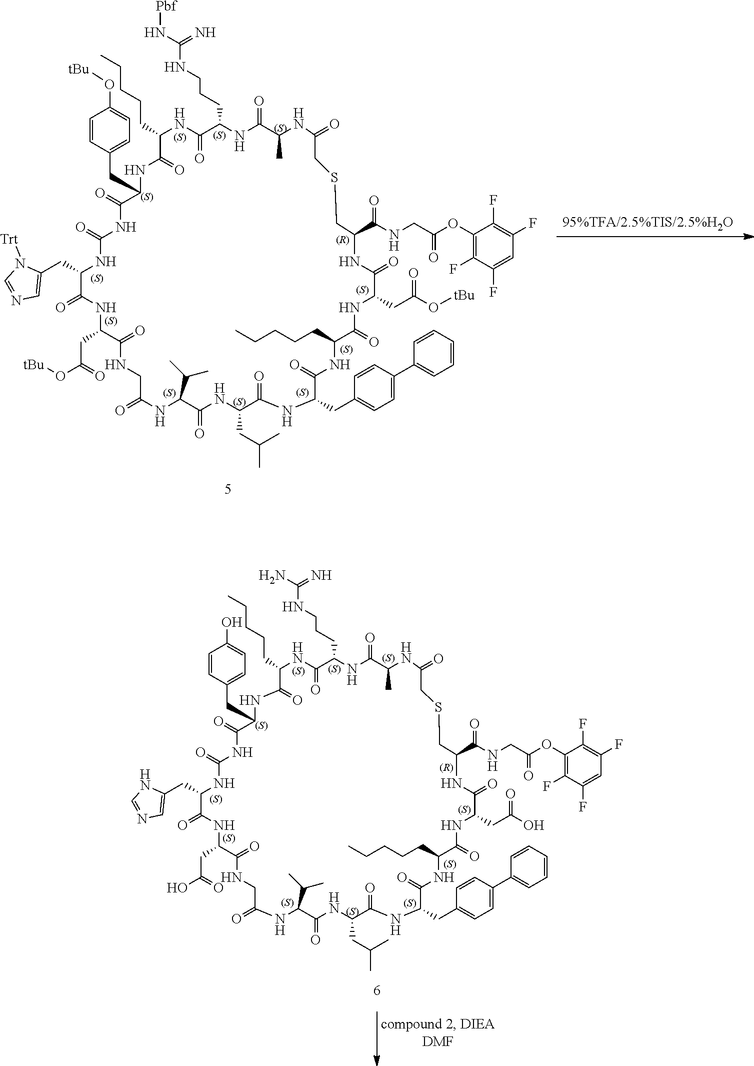

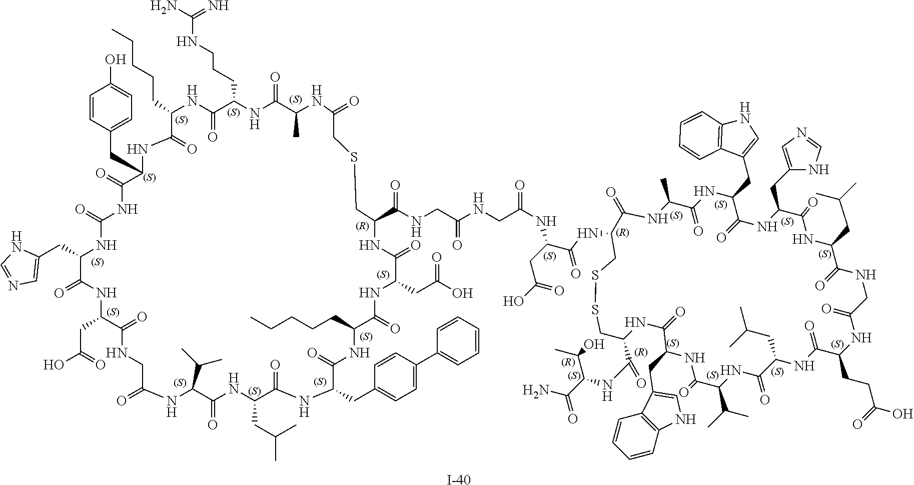

Exemplary Peptide Synthesis. Peptide were synthesized using standard Fmoc chemistry, for example:

1) Resin preparation: To the vessel containing Rink Amide MBHA Resin (0.5 mmol, 1.61 g, 0.31 mmol/g) in DMF (10 mL) with N₂ bubbling at 15° C. for 30 min. Then 20% piperidine in DMF (20 mL) was added and the mixture was bubbled with N₂ for 30 mins at 15° C. Then the mixture was filtered to obtain the resin. The resin was washed with DMF (20 mL)*5 before proceeding to next step.
2) Coupling: A solution of Fmoc-Cys(Trt)-OH (3.00 eq), HBTU (2.85 eq) in DMF (10 mL) was added to the resin with N₂ bubbling. Then DIEA (6.00 eq) was added to the mixture dropwise and bubbled with N₂ for 30 mins at 15° C. The coupling reaction was monitored by ninhydrin test, if it showed colorless, the coupling was complete. The resin was then washed with DMF (20 mL)*5.
3) De-protection: 20% piperidine in DMF (20 mL) was added to the resin and the mixture was bubbled with N₂ for 30 mins at 15° C. The resin was then washed with DMF (20 mL)*5. The De-protection reaction was monitored by ninhydrin test, if it showed blue or other brownish red, the reaction was complete.
4) Repeat Step 2 and 3 for all other amino acids: see below.
5) After the last position completed, the resin was washed with DMF (20 mL)*5, MeOH (20 mL)*5 and then dried under vacuum.

| # | Materials | Coupling reagents |
|---|---|---|
| 1 | Fmoc-Thr(tBu)-OH (3.00 eq) | HBTU (2.85 eq) and DIEA (6.00 eq) |
| 2 | Fmoc-Cys(Trt)-OH (3.00 eq) | HBTU (2.85 eq) and DIEA (6.00 eq) |
| 3 | Fmoc-Trp-OH (3.00 eq) | HBTU (2.85 eq) and DIEA (6.00 eq) |
| 4 | Fmoc-Val-OH (3.00 eq) | HBTU (2.85 eq) and DIEA (6.00 eq) |
| 5 | Fmoc-Leu-OH (3.00 eq) | HBTU (2.85 eq) and DIEA (6.00 eq) |
| 6 | Fmoc-Glu(OtBu)-OH (3.00 eq) | HBTU (2.85 eq) and DIEA (6.00 eq) |
| 7 | Fmoc-Gly-OH (3.00 eq) | HBTU (2.85 eq) and DIEA (6.00 eq) |
| 8 | Fmoc-Leu-OH (3.00 eq) | HBTU (2.85 eq) and DIEA (6.00 eq) |
| 9 | Fmoc-His(Trt)-OH (2.00 eq) | HBTU (2.85 eq) and DIEA (6.00 eq) |
| 10 | Fmoc-Trp-OH (3.00 eq) | HBTU (2.85 eq) and DIEA (6.00 eq) |
| 11 | Fmoc-Ala-OH (3.00 eq) | HBTU (2.85 eq) and DIEA (6.00 eq) |
| 12 | Fmoc-Cys(Trt)-OH (3.00 eq) | HBTU (2.85 eq) and DIEA (6.00 eq) |
| 13 | Fmoc-Asp(OtBu)-OH (3.00 eq) | HBTU (2.85 eq) and DIEA (6.00 eq) |
| 14 | Fmoc-Gly-OH (3.00 eq) | HBTU (2.85 eq) and DIEA (6.00 eq) |

### Peptide Cleavage and Purification:

1) Add cleavage buffer (95% TFA/2.5% TIS/2.5% H₂O, 20 mL) to the flask containing the side chain protected peptide at room temperature and stirred for 1 hr.
2) Filter and collect the filtrate.
3) The peptide is precipitated with cold isopropyl ether (50 mL) and centrifuged (3 min at 3000 rpm).
4) Isopropyl ether washes two additional times, and dry the crude peptide under vacuum for 2 hr.
5) To a mixture of the crude in ACN/H₂O (300 mL) was added 0.1 M I₂/AcOH dropwise until the light yellow persisted, then the mixture was stirred at 15° C. for 5 min. The mixture was quenched with 0.1 M Na₂S₂O₃ dropwise until the light yellow disappeared. The mixture was dried via lyophilization and then the residue was purified by prep-HPLC (acid condition, TFA) directly to get compound 2 (150 mg, 90% purity)

Another exemplary Peptide Synthesis. Peptide were synthesized using standard Fmoc chemistry, for example:

1) Resin preparation: To the vessel containing CTC Resin (0.5 mmol, 0.50 g, 1.0 mmol/g) and Fmoc-Gly-OH (0.149 g, 0.50 mmol, 1.00 eq) in DCM (5 mL) was added DIEA (4.00 eq) dropwise and mix for 2 hr with N₂ bubbling at 15° C. Then added MeOH (0.5 mL) and bubbled with N₂ for another 30 min. The resin was washed with DMF (10 mL)*5, and then 20% piperidine in DMF (10 mL) was added and the mixture was bubbled with N₂ for 30 mins at 15° C. Then the mixture was filtered to obtain the resin. The resin was washed with DMF (10 mL)*5 before proceeding to next step.
2) Coupling: A solution of Fmoc-Cys(Mmt)-OH (2.00 eq), HBTU (1.90 eq) in DMF (5 mL) was added to the resin with N₂ bubbling. Then DIEA (4.00 eq) was added to the mixture dropwise and bubbled with N₂ for 30 min at 15° C. The coupling reaction was monitored by ninhydrin test, if it showed colorless, the coupling was complete. If it showed blue or brownish red, the coupling was repeated one more time and checked with ninhydrin test again till coupling reaches completion. The resin was then washed with DMF (10 mL)*5.
3) De-protection: 20% piperidine in DMF (10 mL) was added to the resin and the mixture was bubbled with N₂ for 30 min at 15° C. The resin was then washed with DMF (10 mL)*5. The De-protection reaction was monitored by ninhydrin test, if it showed blue or other brownish red, the reaction was complete.
4) Repeat Step 2 and 3 for all other amino acids: (2-14 in Table 2).
5) Coupling for the last position: A solution of 2-bromoacetic acid (4.00 eq) and DIC (4.00 eq) was added to resin and the mixture was bubbled with N₂ for 20 mins. The coupling reaction was monitored by ninhydrin test, if it showed colorless, the coupling was completed. The resin was then washed with DMF (10 mL)*5, MeOH (10 mL)*5, and then dried under vacuum.

| # | Materials | Coupling reagents |
|---|---|---|
| 1 | Fmoc-Gly-OH (1.00 eq) | DIEA (4.00 eq) |
| 2 | Fmoc-Cys(Mmt)-OH (2.00 eq) | HATU (1.90 eq) and DIEA (4.00 eq) |
| 3 | Fmoc-Asp(OtBu)-OH (3.00 eq) | HBTU (2.85 eq) and DIEA (6.00 eq) |
| 4 | (S)-2-((((9H-fluoren-9- | HATU (1.35 eq) and DIEA (3.00 eq) |
| | yl)methoxy)carbonyl)amino)heptanoic acid (1.50 eq) | |
| 5 | (S)-2-((((9H-fluoren-9-yl)methoxy)carbonyl)amino)-3- | HATU (1.35 eq) and DIEA (3.00 eq) |
| | ([1,1′-biphenyl]-4-yl)propanoic acid (1.50 eq) | |
| 6 | Fmoc-Leu-OH (3.00 eq) | HBTU (2.85 eq) and DIEA (6.00 eq) |
| 7 | Fmoc-Val-OH (3.00 eq) | HBTU (2.85 eq) and DIEA (6.00 eq) |
| 8 | Fmoc-Gly-OH (3.00 eq) | HBTU (2.85 eq) and DIEA (6.00 eq) |
| 9 | Fmoc-Asp(OtBu)-OH (3.00 eq) | HBTU (2.85 eq) and DIEA (6.00 eq) |
| 10 | Fmoc-His(Trt)-OH (3.00 eq) | HBTU (2.85 eq) and DIEA (6.00 eq) |
| 11 | Fmoc-Tyr(tBu)-OH (1.50 eq) | HBTU (2.85 eq) and DIEA (6.00 eq) |
| 12 | (S)-2-((((9H-fluoren-9-yl)methoxy)carbonyl)amino) | HATU (1.35 eq) and DIEA (3.00 eq) |
| | heptanoic acid (1.50 eq) | |
| 13 | Fmoc-Arg(Pbf)-OH (3.00 eq) | HBTU (2.85 eq) and DIEA (6.00 eq) |
| 14 | Fmoc-Ala-OH (3.00 eq) | HBTU (2.85 eq) and DIEA (6.00 eq) |
| 15 | 2-bromoacetic acid (6.00 eq) | DIC (6.00 eq) |

### Peptide Cleavage and Purification:

1) Add cleavage buffer (2% TFA/2% TIS/96% DCM, 100 mL) to the flask containing the sidechain protected peptide at room temperature with N₂ bubbling for 20 mins.
2) Filter and collect the filtrate. The clear solution is compound 3 (0.5 mmol, 100 mL) in cleavage buffer, and is used in next step directly.
3) Compound 3 (0.5 mmol, 100 mL, in cleavage buffer) was diluted with MeOH (500 mL), based with TEA to pH=8 under N₂ atmosphere, and then stirred at 15° C. for 4 hr. The solvent was removed under reduced pressure, and the residue was triturated in 0.1 M HCl (cold, 100 mL). After filtration, the solid was washed with H₂O (50 mL) and then stirred in isopropyl ether (50 mL) for 10 mins, and finally dried under reduced pressure to get compound 4 (1.2 g, crude).

A mixture of compound 4 (1.2 g, 1.00 eq), compound 4a (501 mg, 6.00 eq), EDCI (288 mg, 3.00 eq) in DMF (10 mL) was stirred at 15° C. for 3 hr. The mixture was then added to 0.5 M HCl (cold, 100 mL), and there appeared lots of white solid. After filtered, the solid was washed with H₂O (20 mL), dried via lyophilization to get compound 5 (1.8 g, crude) as a white solid. The crude was treated with deprotection cocktail (95% TFA/2.5% TIS/2.5% H2O, 30 mL) for 1.5 hr. The mixture was precipitated with cold isopropyl ether (100 mL) and centrifuged (3 min at 3000 rpm). Cold isopropyl ether washed the precipitate two additional times (50 mL). Dry the crude peptide under vacuum 2 hrs. The residue was purified by flash C18 (ISCO®; 120 g SepaFlash® C18 Flash Column, Eluent of 0-90% MeCN/H₂O gradient @ 75 mL/min) directly to get compound 6 (55 mg) as a white solid.

A mixture of compound 6 (55 mg, 1.00 eq), compound 2 (51.3 mg, 1.10 eq), DIEA (30.7 uL, 6.00 eq) in DMF (0.5 mL) was stirred at 15° C. for 1 hr. The solution was purified by prep-HPLC (TFA condition) directly to get compound 523 (15.4 mg, 15.9% yield, 98.6% purity) as a white solid. LCMS: 1098.4 (+ESI Scan; major peak).

Purification Conditions:

Purification condition

| | |
|---|---|
| Sample Preparation | Dissolve in MeCN/H₂O |
| Instrument | Gilson GX-215 |
| Mobile Phase | A: H₂O (0.075% TFA in H₂O) |
| | B: MeCN |
| Gradient | 20-50%-60 min. Retention time: 45 min |
| Column | Luna 25*200 mm, C18 10 um, 110 A + Gemin 150*30 mm, C18 5 um, 110 Å |
| Flow Rate | 20 mL/min |
| Wavelength | 220/254 nm |
| Oven Tem. | Room temperature |

Exemplary Preparation of I-33, I-34, I-35, I-36, I-37, I-41 and I-42.

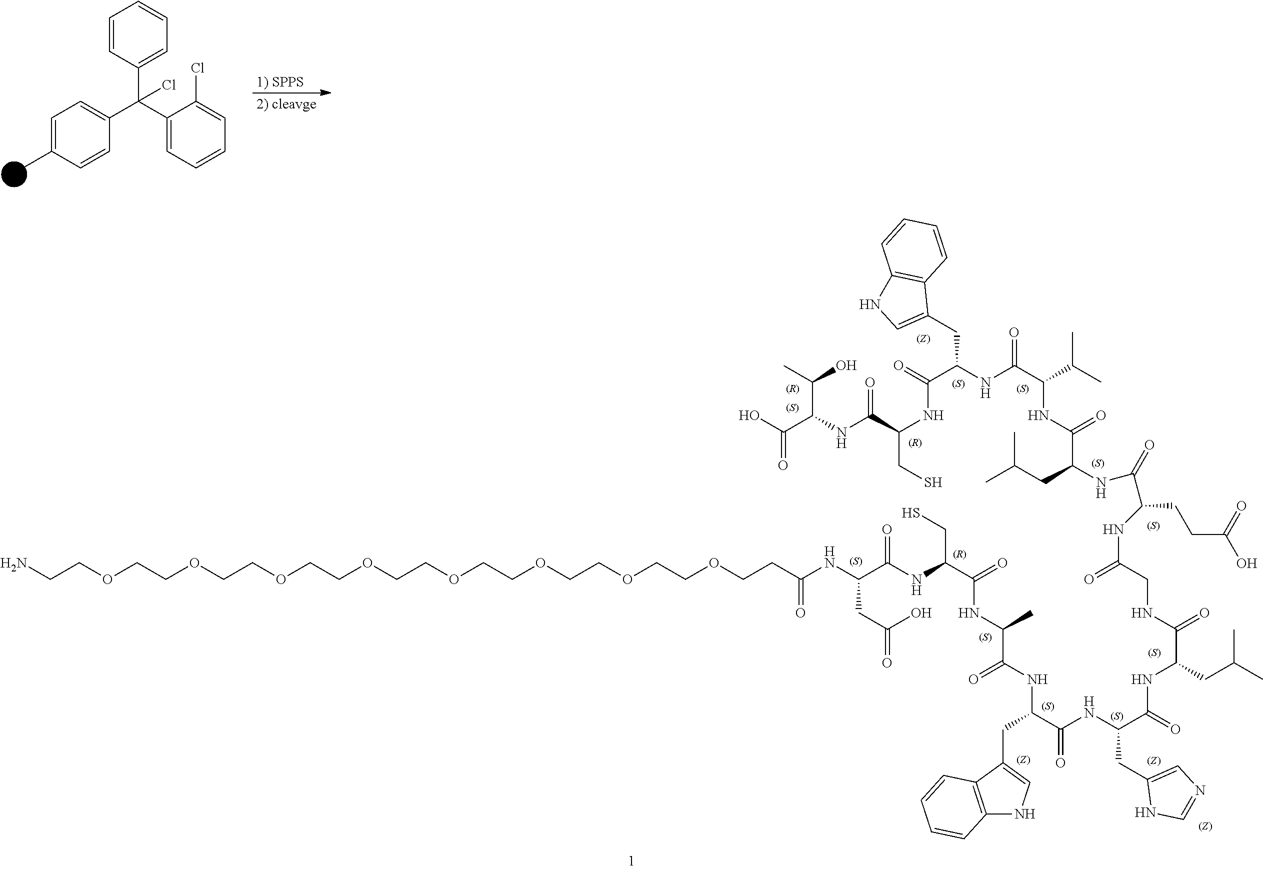

Exemplary Peptide Synthesis. Peptide were synthesized using standard Fmoc chemistry, for example:

1) Resin preparation: To the vessel containing CTC Resin (0.5 mmol, 0.50 g, 1.0 mmol/g, 1.00 eq) and Fmoc-Thr(tBu)-OH (0.158 g, 0.40 mmol, 0.8 eq) in DCM (5 mL) was added DIEA (4.00 eq) dropwise and mix for 2 hr with N₂ bubbling at 15° C. Then added MeOH (0.5 mL) and bubbled with N₂ for another 30 min. The resin was washed with DMF (10 mL)*5. Then 20% piperidine in DMF (10 mL) was added and the mixture was bubbled with N₂ for 30 min at 15° C. Then the mixture was filtered to obtain the resin. The resin was washed with DMF (10 mL)*5 before proceeding to next step.
2) Coupling: A solution of Fmoc-Cys (Trt)-OH (877 mg, 1.5 mmol, 3.00 eq), HBTU (541 mg, 1.42 mmol, 2.85 eq) in DMF (5 mL) was added to the resin with N₂ bubbling. Then DIEA (6.00 eq) was added to the mixture dropwise and bubbled with N₂ for 30 min at 15° C. The coupling reaction was monitored by ninhydrin test, if it showed colorless, the coupling was complete. If it showed blue or brownish red, the coupling was repeated and checked with ninhydrin test again till reaching completion. The resin was then washed with DMF (10 mL)*5.
3) De-protection: 20% piperidine in DMF (10 mL) was added to the resin and the mixture was bubbled with N₂ for 30 mins at 15° C. The resin was then washed with DMF (10 mL)*5. The De-protection reaction was monitored by ninhydrin test, if it showed blue or other brownish red, the reaction was completed.
4) Repeat Step 2 and 3 for all other amino acids: see below.
5) After the last position completed, the resin was washed with DMF (10 mL)*5, MeOH (10 mL)*5 and then dried under vacuum.

| # | Materials | Coupling reagents |
|---|---|---|
| 1 | Fmoc-Thr(tBu)-OH (0.80 eq) | DIEA (4.00 eq) |
| 2 | Fmoc-Cys(Trt)-OH (3.00 eq) | HBTU (2.85 eq) and DIEA (6.00 eq) |
| 3 | Fmoc-Trp(Boc)-OH (3.00 eq) | HBTU (2.85 eq) and DIEA (6.00 eq) |
| 4 | Fmoc-Val-OH (3.00 eq) | HBTU (2.85 eq) and DIEA (6.00 eq) |
| 5 | Fmoc-Leu-OH (3.00 eq) | HBTU (2.85 eq) and DIEA (6.00 eq) |
| 6 | Fmoc-Glu(OtBu)-OH (3.00 eq) | HBTU (2.85 eq) and DIEA (6.00 eq) |
| 7 | Fmoc-Gly-OH (3.00 eq) | HBTU (2.85 eq) and DIEA (6.00 eq) |
| 8 | Fmoc-Leu-OH (3.00 eq) | HATU (2.85 eq) and DIEA (6.00 eq) |
| 9 | Fmoc-His(Trt)-OH (3.00 eq) | HATU (2.85 eq) and DIEA (6.00 eq) |
| 10 | Fmoc-Trp(Boc)-OH (3.00 eq) | HATU (2.85 eq) and DIEA (6.00 eq) |
| 11 | Fmoc-Ala-OH (3.00 eq) | HATU (2.85 eq) and DIEA (6.00 eq) |
| 12 | Fmoc-Cys(Trt)-OH (3.00 eq) | HATU (2.85 eq) and DIEA (6.00 eq) |
| 13 | Fmoc-Asp(OtBu)-OH (3.00 eq) | HATU (2.85 eq) and DIEA (6.00 eq) |
| 14 | Fmoc-PEG8-CH2CH2COOH (2.00 eq) | HATU (1.90 eq) and DIEA (4.00 eq) |

### Peptide Cleavage and Purification:

1) Add cleavage buffer (95% TFA/2.5% TIS/2.5% H₂O) to the flask containing the sidechain protected peptide at room temperature and stirred for 1 hr.
2) Filter and collect the filtrate.
3) The peptide is precipitated with cold isopropyl ether (50 mL) and centrifuged (3 mins at 3000 rpm).
4) Isopropyl ether washes two additional times, and dry the crude peptide under vacuum for 2 hr.
5) To give compound 5 (800 mg, crude) as a white solid.

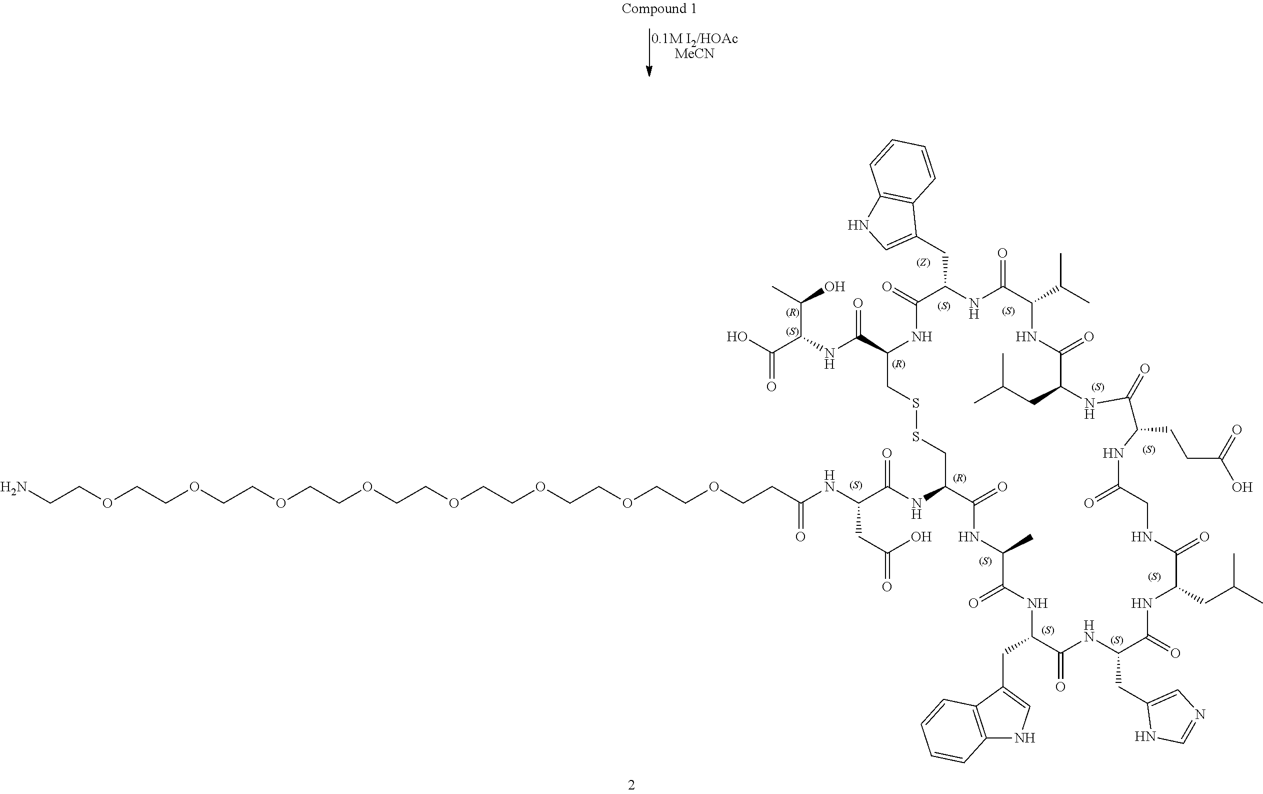

Compound 1 was dissolved in MeCN/H₂O (500 mL), and added 0.1 M I₂/HOAc dropwise until the color of mixture turned to light yellow, then the mixture was stirred at 15° C. for 10 min. The mixture was quenched with 0.1 M Na₂S₂O₃ dropwise until the color of mixture turned to colorless, dried via lyophilization. The residue was purified by prep-HPLC (acid condition, TFA) directly to get compound 2 (170 mg, 90.0% purity, 17.4% yield).

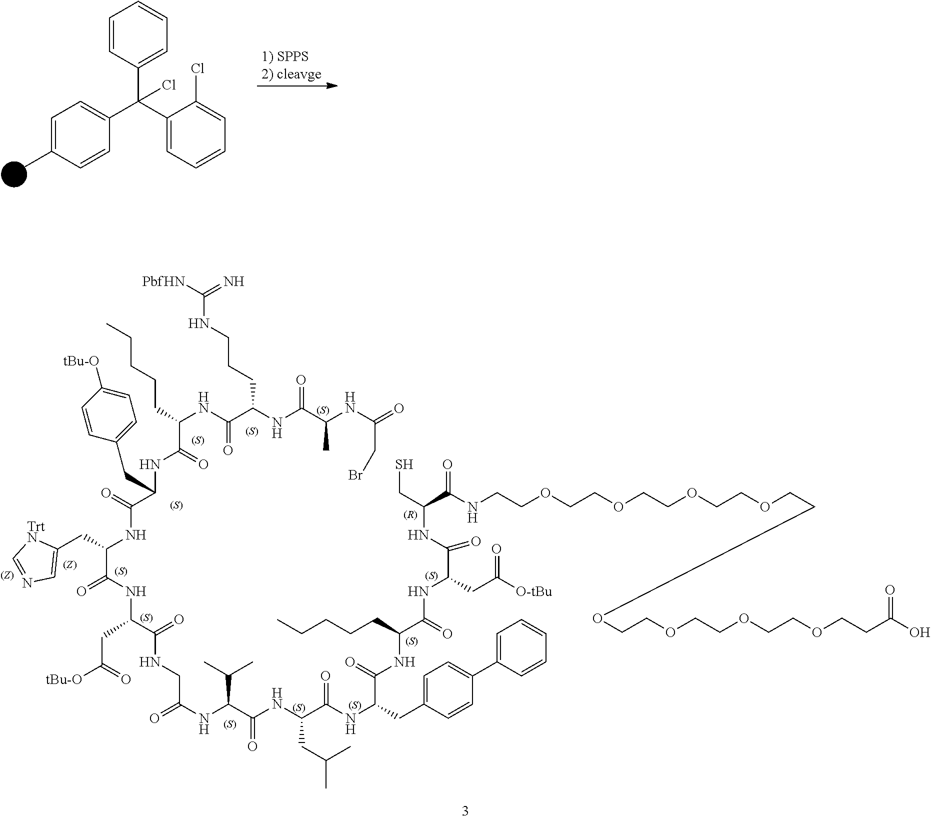

Peptide were synthesized using standard Fmoc chemistry, for example:

1) Resin preparation: To the vessel containing CTC Resin (0.62 mmol, 0.62 g, 1.0 mmol/g, 1.25 eq) and Fmoc-PEGs-CH₂CH₂COOH (0.198 g, 0.50 mmol, 1.00 eq) in DCM (10 mL) was added DIEA (4.00 eq) dropwise and mix for 2 hrs with N₂ bubbling at 15° C. Then added MeOH (1.0 mL) and bubbled with N₂ for another 30 min. The resin was washed with DMF (10 mL)*5. Then 20% piperidine in DMF (10 mL) was added and the mixture was bubbled with N₂ for 30 min at 15° C. Then the mixture was filtered to obtain the resin. The resin was washed with DMF (10 mL)*5 before proceeding to next step.
2) Coupling: A solution of Fmoc-Cys(Mmt)-OH (615 mg, 1.00 mmol, 2.00 eq), HBTU (360 mg, 0.95 mmol, 1.90 eq) in DMF (10 mL) was added to the resin with N₂ bubbling. Then DIEA (4.00 eq) was added to the mixture dropwise and bubbled with N₂ for 30 min at 15° C. The coupling reaction was monitored by ninhydrin test, if it showed colorless, the coupling was complete. If it showed blue or brownish red, the coupling was repeated and checked with ninhydrin test again till reaching completion. The resin was then washed with DMF (20 mL)*5.
3) De-protection: 20% piperidine in DMF (20 mL) was added to the resin and the mixture was bubbled with N₂ for 30 min at 15° C. The resin was then washed with DMF (20 mL)*5. The De-protection reaction was monitored by ninhydrin test, if it showed blue or other brownish red, the reaction was complete.
4) Repeat Step 2 and 3 for all other amino acids: see below.
5) Coupling for the last position: A solution of 2-bromoacetic acid (4.00 eq) and DIC (4.00 eq) was added to resin and the mixture was bubbled with N₂ for 20 min. The coupling reaction was monitored by ninhydrin test, if it showed colorless, the coupling was completed. The resin was then washed with DMF (20 mL)*5, MeOH (20 mL)*5, and then dried under vacuum.

| | | |
|---|---|---|
| # | Materials | Coupling reagents |

| | | |
|---|---|---|
| 1 | Fmoc-PEG₈-CH₂CH₂COOH (1.00 eq) | DIEA (4.00 eq) |
| 2 | Fmoc-Cys(Mmt)-OH (2.00 eq) | HATU (1.90 eq) and DIEA (4.00 eq) |
| 3 | Fmoc-Asp(OtBu)-OH (3.00 eq) | HBTU (2.85 eq) and DIEA (6.00 eq) |
| 4 | (S)-2-((((9H-fluoren-9- | HATU (1.35 eq) and DIEA (3.00 eq) |
| | yl)methoxy)carbonyl)amino)heptanoic acid (1.50 eq) | |
| 5 | (S)-2-((((9H-fluoren-9-yl)methoxy)carbonyl)amino)-3- | HATU (1.35 eq) and DIEA (3.00 eq) |
| | ([1,1′-biphenyl]-4-yl)propanoic acid (1.50 eq) | |
| 6 | Fmoc-Leu-OH (3.00 eq) | HBTU (2.85 eq) and DIEA (6.00 eq) |
| 7 | Fmoc-Val-OH (3.00 eq) | HBTU (2.85 eq) and DIEA (6.00 eq) |
| 8 | Fmoc-Gly-OH (3.00 eq) | HBTU (2.85 eq) and DIEA (6.00 eq) |
| 9 | Fmoc-Asp(OtBu)-OH (3.00 eq) | HBTU (2.85 eq) and DIEA (6.00 eq) |
| 10 | Fmoc-His(Trt)-OH (3.00 eq) | HBTU (2.85 eq) and DIEA (6.00 eq) |
| 11 | Fmoc-Tyr(tBu)-OH (1.50 eq) | HBTU (2.85 eq) and DIEA (6.00 eq) |
| 12 | (S)-2-((((9H-fluoren-9- | HATU (1.35 eq) and DIEA (3.00 eq) |
| | yl)methoxy)carbonyl)amino)heptanoic acid (1.50 eq) | |
| 13 | Fmoc-Arg(Pbf)-OH (3.00 eq) | HBTU (2.85 eq) and DIEA (6.00 eq) |
| 14 | Fmoc-Ala-OH (3.00 eq) | HBTU (2.85 eq) and DIEA (6.00 eq) |
| 15 | 2-bromoacetic acid (4.00 eq) | DIC (4.00 eq) |

### Peptide Cleavage and Purification:

1) Add cleavage buffer (2% TFA/2% TIS/96% DCM, 100 mL) to the flask containing the side chain protected peptide at room temperature with N₂ bubbling for 20 min.
2) Filter and collect the filtrate. The clear solution is compound 3 (1.0 mmol, 100 mL) in cleavage buffer, and is used in next step directly.

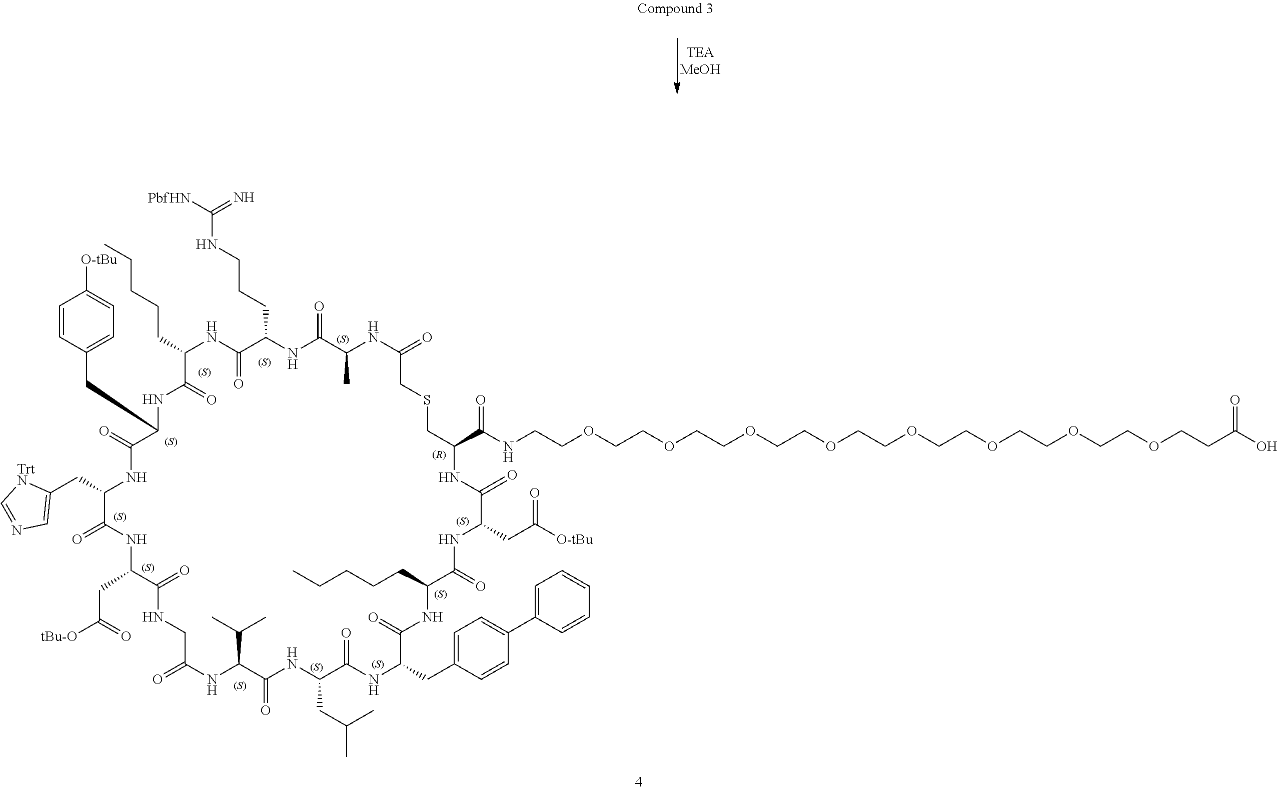

Compound 3 (1.0 mmol, 100 mL, in cleavage buffer) was diluted with MeOH (1000 mL), based with TEA to pH=8 under N₂ atmosphere, and then stirred at 15° C. for 2 hr. The solvent was removed under reduced pressure, and the residue was triturated in 0.1 M HCl (cold, 100 mL). After filtration, the solid was washed with H₂O (50 mL), and then stirred in isopropyl ether (50 mL) for 10 min. Finally, the solid was dried under reduced pressure to get compound 4 (0.75 g, crude).

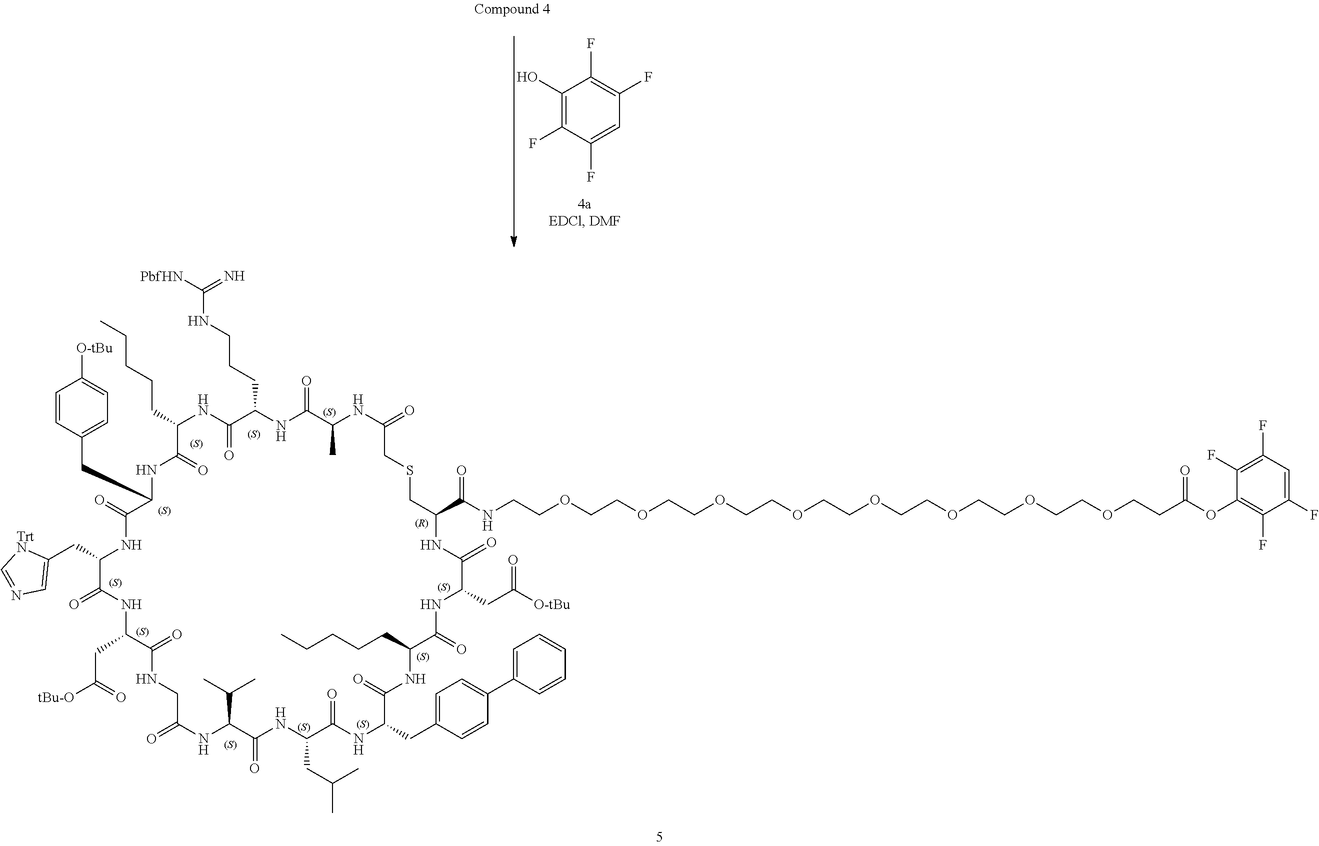

A mixture of compound 4a (226.2 mg, 1.36 mmol, 5.00 eq), compound 4 (0.75 g, 272.4 umol, 1.00 eq), EDCI (156.71 mg, 817.48 umol, 3.00 eq) in DMF (5 mL) was stirred at 15° C. for 3 hr. The mixture was added to 0.5 M HCl (cold, 50 mL), there appeared lots of white solid. After filtration, the solid was washed with H₂O (20 mL), dried via lyophilization to get compound 5 (0.80 g, crude) as a white solid.

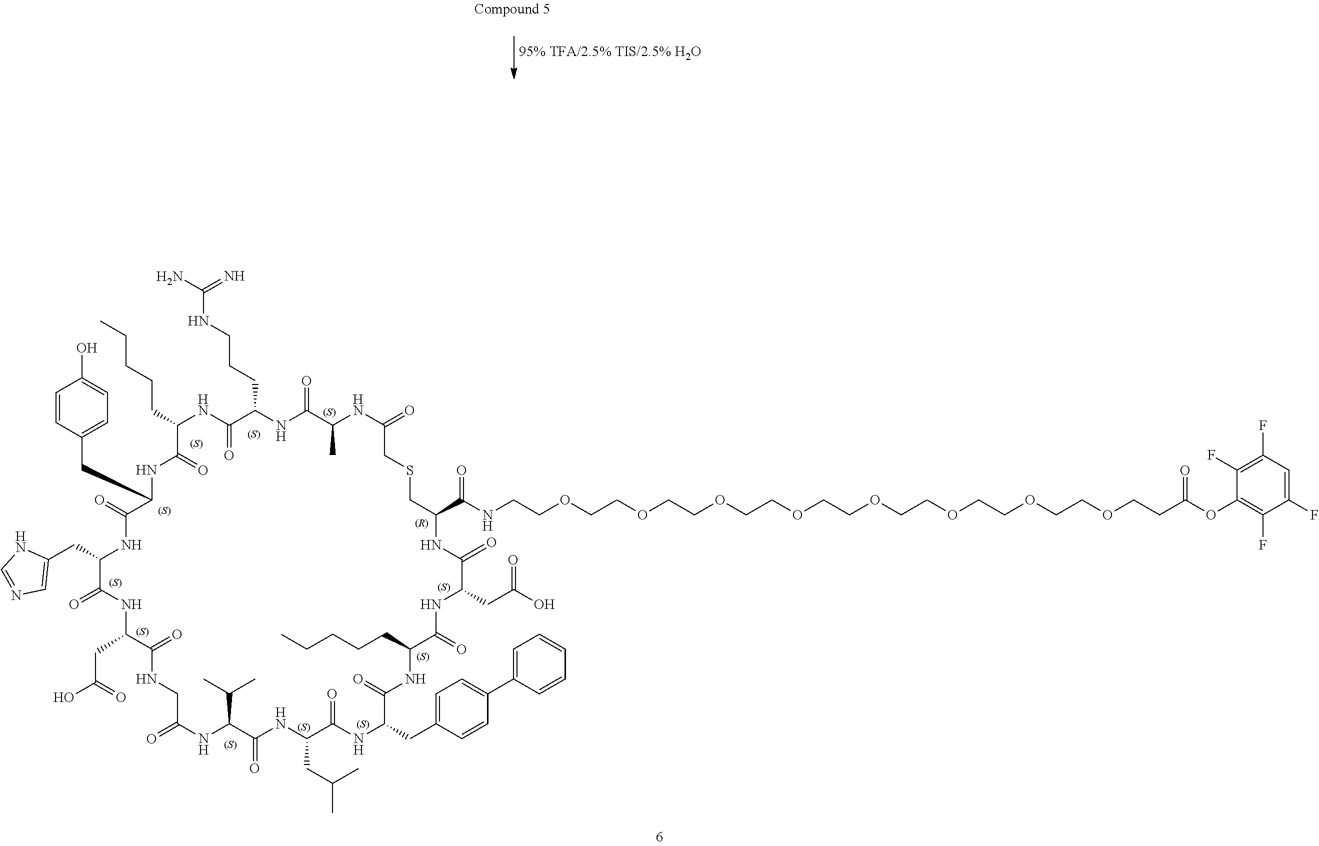

Compound 5 (0.80 g, crude) was dissolved in protection cocktail containing TFA (17.6 g, 154.3 mmol, 11.4 mL), H₂O (285.7 mg, 15.8 mmol, 285 uL), and triisopropylsilane (220.29 mg, 1.39 mmol, 285.71 uL), and the mixture was stirred at 15° C. for 1 hr. The mixture was then precipitated with cold isopropyl ether (100 mL) and centrifuged (3 mins at 3000 rpm). Cold isopropyl ether washed the precipitate two additional times (50 mL). Dry the crude peptide under vacuum 2 hr. The residue was purified by flash C18 (ISCO®; 120 g SepaFlash® C18 Flash Column, Eluent of 0-90% MeCN/H₂O gradient @ 75 mL/min) directly to get compound 6 (90 mg, 40.2 umol, 14.5% yield, 90.0% purity) as a white solid.

A mixture of compound 6 (50 mg, 22.3 umol, 1.00 eq), compound 2 (43.60 mg, 22.3 umol, 1.00 eq), DIEA (23.1 mg, 178.7 umol, 31.1 uL, 8.00 eq) in DMF (0.5 mL) was stirred at 15° C. for 1 hr. The solution was purified by prep-HPLC (acid condition, TFA) directly to get I-33 (33.0 mg, 7.71 umol, 34.4% yield, 93.9% purity) as a white solid.

Purification Conditions:

Purification condition

| | |
|---|---|
| Sample Preparation | Dissolve in MeCN/H₂O |
| Instrument | Gilson GX-215 |
| Mobile Phase | A: H₂O (0.075% TFA in H₂O) |
| | B: MeCN |
| Gradient | 30-60%-60 min. Retention time: 42 min |
| Column | Luna25*200 mm, C18 10 um, 110A + Gemin150*30 mm, C18 5 um, 110A° |
| Flow Rate | 20 mL/min |
| Wavelength | 220/254 nm |
| Oven Tem. | Room temperature |

I-34, I-35, I-36, I-37, I-41 and I-42 were prepared similarly. Certain results from certain preparations were provided below:

| Compound | LC/MS (+ESI; major peak) | Purifiy (HPLC) |
|---|---|---|
| I-33 | 1342.7 | 94% |
| I-34 | 1166.6 | 96% |
| I-35 | 1195.8 | 97% |
| I-36 | 1254.6 | 97% |
| I-37 | 1313.3 | 96% |
| I-41 | 1366.5 | 92% |
| I-42 | 1361.6 | 91% |

Various other compounds, including various ARM agents, were also prepared and assessed, in many instances, using one or more technologies described in the Examples.

### Example 2. Provided Compounds Recruit Antibodies to Target Cells

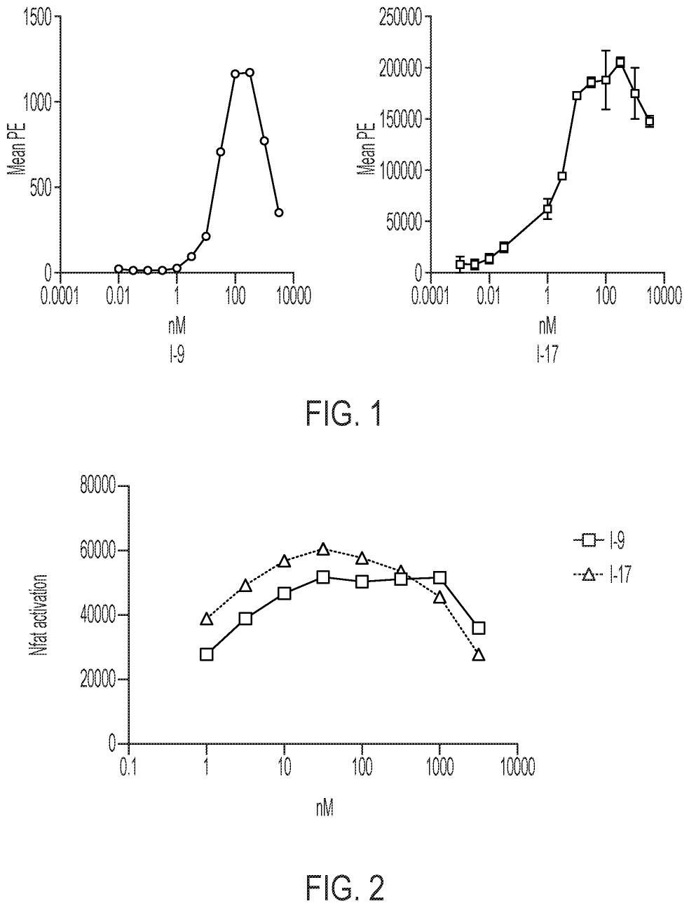

Various technologies can be utilized to assess and/or characterize recruitment of antibodies to target cells, e.g., cancer cells. One such assay is a ternary binding assay. An exemplary protocol is described herein. Compounds were diluted in DMSO (MP 191418) to 1000× the starting concentration used in the assay into a 96 well polypropylene plate (Corning 3357). They were then serially diluted in ½ log increments to generate 8-12 concentrations in DMSO (assay dependent). These DMSO stocks were then step diluted 1/100 into PBS (VWR Cat. #20012043). Step diluted compound range was then added into the polypropylene assay plate 1/10 the volume of the assay volume. Daudi (ATCC CCL-213), a non-adherent cell line, were counted and centrifuged and resuspended at a concentration of 100,000 cells per 90 ul in Flow buffer: 1% BSA (American Bio AB01088-00100); 0.5 mM EDTA (VWR 45001-122); PBS (VWR Cat. #20012043) with 11.1 ug/mL human IgG1k Phycoerythrin labeled (SouthernBiotech #0151K-09 PE). The cells were then added into the polypropylene plate with the step diluted compounds and incubated at 37° C. for 30 min. At the end of the incubation, the cells were centrifuged and washed 2× with How buffer containing 0.5% Tween 20 (BP337-500). Samples were analyzed on a BD FacsCelesta. Mean fluorescence was analyzed using Graphpad Prism and curves were fit using log(inhibitor) vs. response—Variable slope (four parameters). As shown in FIG. 1, provided compounds can recruit antibodies to target cells. In one set of experiments, IC50 for I-9 is about 16 nM, and for I-17 is about 2.6 nM. Properties and/or activities of provided technologies were confirmed by assessment of additional compounds. For example, various provided compounds can recruit antibodies to target cells as shown below (MFI at various concentrations):

| ID | 3000 nM | 3000 nM | 1000 nM | 1000 nM | 300 nM | 300 nM | 100 nM | 100 nM |
|---|---|---|---|---|---|---|---|---|
| I-9 | 4383 | 4301 | 4929 | 5108 | 7835 | 7873 | 9899 | 10608 |
| I-17 | 9069 | 9922 | 10015 | 9697 | 7514 | 8562 | 7575 | 6333 |
| I-25 | | | 9220 | 10816 | 8878 | 10199 | 8624 | 11567 |
| I-26 | | | 8503 | 10012 | 7861 | 9489 | 9554 | 9154 |
| I-27 | 3939 | 5161 | 2046 | 10122 | 7177 | 11100 | 11543 | 10823 |
| I-28 | 5225 | 5328 | 5146 | 5377 | 6237 | 5317 | 5106 | 5379 |
| I-29 | 5418 | 6788 | 3906 | 6068 | 3629 | 4497 | 1847 | 2138 |
| I-31 | 9010 | 11779 | 3602 | 6438 | 2442 | 2165 | 2693 | 3081 |
| I-32 | 22956 | 26077 | 28529 | 32355 | 26567 | 20604 | 13843 | 19475 |
| I-33 | 28412 | 12352 | 17428 | 22220 | 24105 | 34283 | 31865 | 24220 |
| I-34 | 19464 | 23695 | 27313 | 25699 | 24939 | 24944 | 30043 | 19748 |
| I-35 | 18010 | 18005 | 27135 | 27638 | 27741 | 24847 | 32049 | 29219 |
| I-36 | 19404 | 27714 | 36394 | 34737 | 27283 | 27320 | 37008 | 29495 |
| I-37 | 23638 | 26361 | 26802 | 26666 | 33948 | 39639 | 30110 | 35481 |
| I-39 | 10149 | 15038 | 9707 | 11970 | 12197 | 16878 | 16375 | 17274 |
| I-40 | 7332 | 6676 | 7288 | 7253 | 7336 | 7421 | 6093 | 6882 |
| I-42 | 13519 | 15909 | 20476 | 15023 | 14190 | 18622 | 16690 | 17157 |
| I-41 | 18339 | 21903 | 13982 | 11893 | 23096 | 16482 | 19188 | 14043 |
| I-43 | 6831 | 6174 | 6763 | 6567 | 5333 | 5056 | 2758 | 3596 |
| I-44 | 5266 | 6972 | 6558 | 6800 | 6141 | 7474 | 4665 | 4868 |
| ID | 30 nM | 30 nM | 10 nM | 10 nM | 3 nM | 3 nM | I nM | 1 nM |
| I-9 | 10630 | 8384 | 7133 | 6933 | 4268 | 4210 | 3063 | 2925 |
| I-17 | 3869 | 5244 | 7119 | 4086 | 2516 | 2680 | 3272 | 2451 |
| I-25 | 9678 | 9210 | 6764 | 6686 | 3191 | 4001 | 3408 | 3932 |
| I-26 | 6606 | 8726 | 4866 | 5842 | 775 | 4521 | 4369 | 2846 |
| I-27 | 7882 | 8234 | 3323 | 9772 | 3952 | 3973 | 3132 | 2453 |
| I-28 | 4033 | 4735 | 1967 | 1940 | 400 | 751 | 216 | 385 |
| I-29 | 121 | 202 | 280 | 192 | 114 | 331 | | |
| I-31 | 1195 | 1517 | 2345 | 1526 | 2685 | 2026 | 1654 | 1933 |
| I-32 | 4353 | 2818 | 2888 | 1819 | 1691 | 1627 | 2313 | 1770 |
| I-33 | 23689 | 23673 | 21149 | 13632 | 3661 | 1846 | 3472 | 3689 |
| I-34 | 15113 | 15626 | 11368 | 6266 | 2094 | 1853 | 2288 | 884 |
| I-35 | 26138 | 18787 | 17910 | 9609 | 2749 | 2984 | 4002 | 3046 |
| I-36 | 29693 | 22951 | 18129 | 13279 | 5567 | 4261 | 2423 | 2071 |
| I-37 | 32775 | 26804 | 25582 | 23068 | 9459 | 9952 | 6516 | 9479 |
| I-39 | 9350 | 9465 | 5298 | 7845 | 6081 | 8739 | 4577 | 5155 |
| I-40 | 2161 | 2640 | 1951 | 1964 | 900 | 1069 | 1098 | 966 |
| I-42 | 9700 | 15077 | 6189 | 11302 | 6000 | 5101 | 4981 | 6900 |
| I-41 | 10036 | 10734 | 9033 | 5065 | 5283 | 6064 | 5751 | 3841 |
| I-43 | 2148 | 1806 | 1373 | 1602 | 908 | 999 | 1311 | 832 |
| I-44 | 2082 | 2157 | 625 | 1051 | 792 | 852 | 562 | 887 |
| ID | 0.3 nM | 0.3 nM | 0.1 nM | 0.1 nM | 0.03 nM | 0.03 nM | | |
| I-9 | 2861 | 2727 | 2514 | 2495 | 2297 | 2037 | | |
| I-17 | 2387 | 2457 | 2322 | 2622 | 2516 | 2391 | | |
| I-25 | 2451 | 3306 | 3735 | 2750 | 2989 | 3054 | | |
| I-26 | 2944 | 2485 | 2390 | 2373 | 3431 | 2849 | | |
| I-27 | 3560 | 3194 | 2881 | 2429 | 2141 | 3877 | | |
| I-28 | | | | | | | | |
| I-29 | | | | | | | | |
| I-31 | 2498 | 2456 | | | | | | |
| I-32 | 1988 | 1769 | | | | | | |
| I-33 | 3653 | 687 | | | | | | |
| I-34 | 4969 | 751 | | | | | | |
| I-35 | 3797 | 1282 | | | | | | |
| I-36 | 7171 | 2781 | | | | | | |
| I-37 | 4354 | 4112 | | | | | | |
| I-39 | 2903 | 10225 | 6763 | 4606 | 5280 | 4131 | | |
| I-40 | 1147 | 1287 | 956 | 1171 | 819 | 477 | | |
| I-42 | 3879 | 4284 | 5737 | 4704 | 7077 | 5990 | | |
| I-41 | 3076 | 2422 | 7612 | 4790 | 4489 | 2739 | | |
| I-43 | 599 | 656 | 766 | 684 | 182 | 503 | | |
| I-44 | 847 | 917 | 560 | 624 | 444 | 476 | | |

IC50 (nM) from Certain Assessments as Examples:

| ID | IC50 (nM) |
|---|---|
| I-9 | 16 |
| I-17 | 5.9 |
| I-25 | 9.5 |
| I-26 | 14 |
| I-27 | 6 |
| I-28 | 15 |
| I-29 | 61 |
| I-30 | 14 |
| I-31 | >3000 |
| I-32 | 88 |
| I-33 | 9.3 |
| I-34 | 20 |
| I-35 | 10 |
| I-36 | 10 |
| I-37 | 7.1 |
| I-38 | >3000 |
| I-39 | 3.3 |
| I-40 | 50 |
| I-42 | 2.2 |
| I-41 | 3 |
| I-43 | 43 |
| I-44 | 19 |

Effective recruitment of effector cells in the presence of IgG to various other cell types were confirmed by additional assays.

As described herein, in some embodiments, provided compounds bind to CD38 as measured by SPR with a Kd no more than 200, 100, 50, 40, 30, 20, 10 or 5 nM, and/or bind to antibodies as measured by SPR with a Kd no more than 200, 100, 50, 40, 30, 20, 10 or 5 nM. In some embodiments, it was shown that provided compounds, e.g., I-9 and I-17, can bind to CD38 with Kd of about 10 nM and to human IgG1 and IgG2 with Kd of about 10-20 nm by SPR. Provided compounds were shown to bind to several IgG isotypes with nanomolar Kd binding affinity.

### Example 3. Provided Compounds Recruit and Activate Effector Cells

Daudi (ATCC CCL-213) cells were resuspended in Opti-MEM (Thermo Fisher Scientific #31985070), counted (Life Technologies Countless II cell counter) and plated (5,000 cells in 25 uL/well; Corning plates: 3917). Compounds w diluted in DMSO (MP 191418) to 1000× the starting concentration used in the assay into a 96 well polypropylene plate (Corning 3357). They were then serially diluted in ½ log increments to generate 8 concentrations in DMSO (assay dependent). These DMSO stocks were then step diluted 1/250 into Opti-MEM. Step diluted compound range was then added into the polypropylene assay plate 25 uL/well. The human antibody solution (Grifols IVIG Fleogogamma #61953-0005-2) was diluted to 40 ug/mL and added 25 uL/well. Effector cells (ADCC reporter cells—Promega kit: G7018) were added 37,500 in 25 uL/well. The assay was then incubated 18 h at 37° C. Following the induction period, the plate was equilibrated to 25° C. followed the addition of the luciferase substrate (75 uL/well, 1 vial in 10 mL of Bio-Glo assay buffer, Promega kit: G7018). The luminescence was measured (Biotek Synergy H1 microplate reader). Exemplar data from one set of experiment were presented in FIG. 2, where EC70 for I-9 is about 0.92 nm and for I-17 is about 2.7 nm. Various assays including animal models confirmed that various types of effector cells, such as macrophages, NK cells, etc., can be recruited and activated. As demonstrated, provided compounds can recruit and activate effector cells. Assessment of additional compounds confirmed properties and/or activities of provided technologies. For example, various provided compounds can recruit and activate effector cells (RLU at various concentrations):

| ID | 3000 nM | 3000 nM | 1000 nM | 1000 nM | 300 nM | 300 nM |
|---|---|---|---|---|---|---|
| I-9 | | | 51790 | 65856 | 55313 | 76306 |
| I-17 | | | 60578 | 59440 | 69068 | 69848 |
| I-25 | | | 64316 | 66742 | 69914 | 74649 |
| I-26 | | | 65643 | 67491 | 72439 | 68444 |
| I-27 | | | 77063 | 67973 | 87081 | 87520 |
| I-28 | | | 29019 | 30900 | 28467 | 31578 |
| I-29 | | | 25446 | 23414 | 27666 | 27282 |
| I-30 | 19456 | 17760 | 22085 | 21589 | 22074 | 21976 |
| I-31 | 7049 | 6977 | 5864 | 5605 | 5597 | 5057 |
| I-32 | 27969 | 28421 | 29139 | 30819 | 29471 | 27566 |
| I-33 | 17765 | 17874 | 30912 | 31498 | 39085 | 37532 |
| I-34 | 8601 | 9733 | 12953 | 13376 | 19948 | 20759 |
| I-35 | 8815 | 8350 | 14926 | 15128 | 22930 | 22904 |
| I-36 | 13481 | 13775 | 21119 | 23429 | 30810 | 31945 |
| I-37 | 16450 | 15761 | 32811 | 27821 | 37628 | 35569 |
| I-39 | | | 29690 | 30305 | 37816 | 37983 |
| I-40 | | | 11161 | 10021 | 10850 | 10917 |
| I-42 | | | 27516 | 28425 | 39826 | 37835 |
| I-41 | | | 29516 | 26828 | 38409 | 39619 |
| I-43 | | | 24875 | 23660 | 31212 | 30518 |
| I-44 | | | 8447 | 9800 | 13687 | 13433 |
| ID | 100 nM | 100 nM | 30 nM | 30 nM | 10 nM | 10 nM |
| I-9 | 60746 | 68867 | 55170 | 59273 | 35274 | 44761 |
| I-17 | 70908 | 75233 | 69329 | 70980 | 63014 | 62702 |
| I-25 | 68791 | 68485 | 65822 | 65101 | 59971 | 58373 |
| I-26 | 68343 | 64223 | 64300 | 67175 | 61322 | 59309 |
| I-27 | 78637 | 83212 | 79211 | 80152 | 62454 | 64829 |
| I-28 | 29777 | 30897 | 28233 | 28798 | 14444 | 14908 |
| I-29 | 26752 | 27780 | 28811 | 26826 | 20457 | 19130 |
| I-30 | 20600 | 19749 | 17189 | 17066 | 11442 | 11535 |
| I-31 | 4334 | 4309 | 3970 | 3888 | 3844 | 3801 |
| I-32 | 21760 | 21761 | 9331 | 10103 | 4774 | 4710 |
| I-33 | 37724 | 38924 | 32485 | 34878 | 28854 | 30376 |
| I-34 | 19999 | 21759 | 16833 | 17160 | 13353 | 13815 |
| I-35 | 24388 | 24222 | 21245 | 23519 | 17525 | 18503 |
| I-36 | 32172 | 32243 | 28718 | 30788 | 27520 | 27793 |
| I-37 | 35484 | 34271 | 36603 | 36031 | 33438 | 34888 |
| I-39 | 36487 | 36994 | 34513 | 37067 | 30280 | 28999 |
| I-40 | 11610 | 11279 | 10738 | 10511 | 9927 | 9348 |
| I-42 | 40344 | 39296 | 36909 | 40202 | 32317 | 32373 |
| I-41 | 40904 | 39344 | 35410 | 36310 | 26140 | 28413 |
| I-43 | 31566 | 28250 | 22529 | 22418 | 15615 | 13921 |
| I-44 | 13291 | 12548 | 12041 | 12726 | 6039 | 5790 |
| ID | 3 nM | 3 nM | 1 nM | 1 nM | 0.3 nM | 0.3 nM |
| I-9 | 24006 | 24206 | 20777 | 20342 | 18458 | 20362 |
| I-17 | 50671 | 54979 | 32303 | 36226 | 26632 | 25947 |
| I-25 | 37540 | 37828 | 31200 | 31465 | 22209 | 22720 |
| I-26 | 49751 | 50606 | 34061 | 37470 | 28809 | 28970 |
| I-27 | 54244 | 61633 | 39142 | 41236 | 32081 | 33576 |
| I-28 | 10820 | 11409 | 8149 | 8745 | | |
| I-29 | 11913 | 13153 | 11207 | 11311 | | |
| I-30 | 4269 | 4452 | 4174 | 4217 | | |
| I-31 | 3785 | 4102 | 4065 | 3975 | | |
| I-32 | 4124 | 4275 | 4330 | 4253 | | |
| I-33 | 13089 | 12353 | 5245 | 5460 | | |
| I-34 | 5459 | 5627 | 4355 | 4667 | | |
| I-35 | 7809 | 7907 | 5094 | 4840 | | |
| I-36 | 12341 | 12684 | 6729 | 7167 | | |
| I-37 | 20376 | 20151 | 13516 | 13786 | | |
| I-39 | 17088 | 16702 | 9050 | 9194 | 4863 | 4935 |
| I-40 | 7731 | 8254 | 4341 | 4003 | 1843 | 1786 |
| I-42 | 20721 | 20652 | 13795 | 13686 | 4468 | 4661 |
| I-41 | 15011 | 15516 | 6793 | 6656 | 4814 | 4964 |
| I-43 | 3384 | 3253 | 2778 | 2590 | 2526 | 2121 |
| I-44 | 3366 | 3419 | 2564 | 2631 | 2075 | 1959 |

EC50 (nM) from Certain Assessments as Examples:

| ID | EC50 (nM) |
|---|---|
| I-9 | 5.5 |
| I-17 | 5.9 |
| I-25 | 4.7 |
| I-26 | 2.4 |
| I-27 | 3 |
| I-28 | 14 |
| I-29 | 9.9 |
| I-30 | 12 |
| I-31 | >3000 |
| I-32 | 63 |
| I-33 | 4.3 |
| I-34 | 6.7 |
| I-35 | 4.4 |
| I-36 | 3.4 |
| I-37 | 1.8 |
| I-38 | 53 |
| I-39 | 0.61 |
| I-40 | 50 |
| I-42 | 1.4 |
| I-41 | 24 |
| I-43 | 11 |
| I-44 | 11 |

### Example 4. Provided Compounds can Kill Target Cells

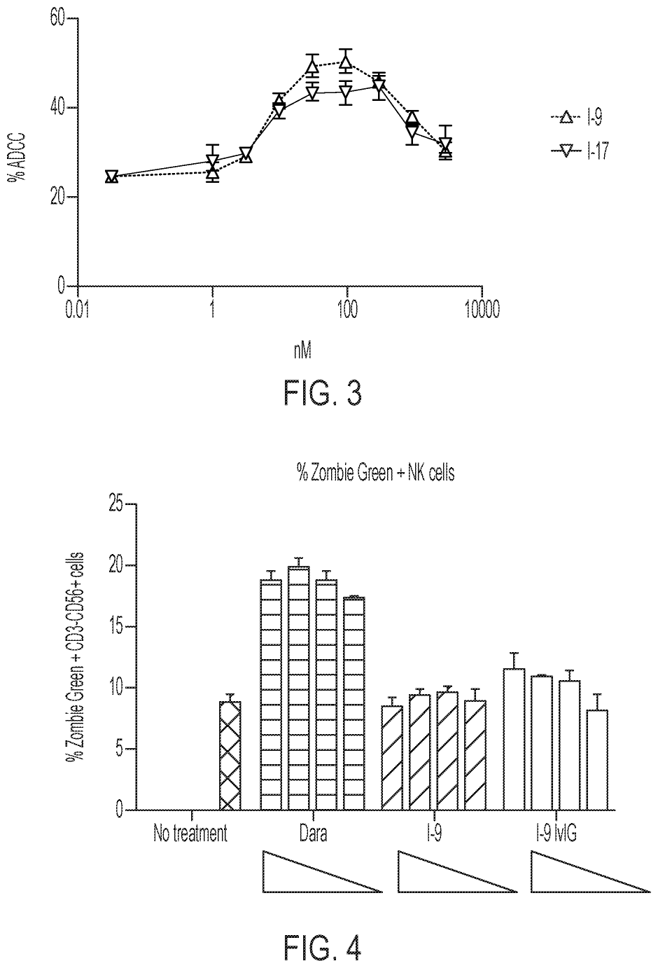

In some embodiments, CD38-ABT ARMs bind to target cells and antibodies, e.g., human antibodies. In some embodiments, such ternary structures present antibodies to effector cells such as NK cell and bind to, e.g., CD16a receptors. Using purified NK cells from multiple donors with different single nucleotide polymorphism variants (V/V, F/F, and V/F) of CD16a experiments were performed to confirm activities of provided compounds, e.g., ARMs, in a system similar to a physiological system. Daudi cells were transfected with KILR reporter construct (DiscoverX #97-0002). An attenuated lentivirus was designed to deliver a Moloney Murine Leukemia Virus (MMLV) engineered to drive expression of a housekeeping gene tagged with Enhanced ProLabel (ePL) a beta-Galactosidase (beta-gal) reporter fragment. This construct was designed to remain inside cells. Death leads to the release of the KILR reporter protein into the medium. Detection reagent (DiscoverX #97-0001L) contained the complementing beta-gal reporter fragment enzyme acceptor (EA). The two components combined to generate a chemiluminescent signal. Data from one set of experiments were presented in FIG. 3, wherein EC50 for I-9 is 6.7 nm and for I-17 is 5.9 nm. It was confirmed that, as described herein, provided agents can recruit effector cells from various sources to kill various CD38-expressing target cells associated with various conditions, disorders or diseases (e.g., cancers such as multiple myeloma), often with EC50 in the low micromolar, nanomolar or low nanomolar ranges. In some embodiments, provided compounds can kill multiple myeloma cells ex vivo in patient bone marrow samples as well as plasma cell leukemia in patient blood. In some embodiments, animal models (e.g., those for Burkitt lymphoma, multiple myeloma, etc.) were utilized to confirm efficacy of provided compounds. As demonstrated, provided compounds can effectively induce killing of target cells, e.g., through recruitment of antibodies and ADCC. Properties and/or activities of provided technologies were confirmed by assessment of additional compounds. For example, various provided compounds can provide activities, in various instances through or comprising ADCC, toward target cells (% dead cells):

| ID | 3000 nM | 3000 nM | 1000 nM | 1000 nM | 300 nM | 300 nM | 100 nM | 100 nM |
|---|---|---|---|---|---|---|---|---|
| I-9 | | | 6.8 | 6.7 | 7.3 | 8.1 | 6.6 | 7.4 |
| I-17 | | | 6.6 | 7.5 | 8.5 | 9.6 | 8.9 | 10.1 |
| I-25 | | | 8.8 | 9.2 | 8.4 | 8.7 | 8.6 | 8.3 |
| I-26 | | | 7.8 | 7 | 8.9 | 7.6 | 7.8 | 7.9 |
| I-27 | | | 7 | 7.1 | 9 | 9.5 | 10.3 | 9.1 |
| I-28 | 9.9 | 10.8 | 13 | 13.4 | 15 | 15.2 | 16.1 | 15.8 |
| I-29 | | | 11.8 | 11.8 | 11.9 | 10.2 | 10.4 | 10.3 |
| I-30 | 18.1 | 17.5 | 19.2 | 20 | 19 | 20.6 | 17.1 | 18.1 |
| I-31 | 13.6 | 13.3 | 14.5 | 13.4 | 14.6 | 13.7 | 13.6 | 13.2 |
| I-32 | 16.9 | 16.8 | 19.7 | 20.4 | 19.5 | 20.1 | 17.7 | 17.2 |
| I-33 | 15.3 | 16.6 | 22.6 | 23.3 | 31.9 | 31.6 | 32.8 | 31.3 |
| I-34 | 14.2 | 13.5 | 15.3 | 15.9 | 18.9 | 18.8 | 19.7 | 16.9 |
| I-35 | 14 | 13.9 | 14.5 | 16.1 | 18.8 | 20.1 | 20 | 22 |
| I-36 | 14.5 | 16 | 16.1 | 17.6 | 24.4 | 25.4 | 25.6 | 25.2 |
| I-37 | 14.7 | 15.1 | 21.1 | 21.2 | 29.1 | 27.4 | 28.6 | 33.2 |
| I-38 | 10.3 | 10.8 | 11.4 | 11.7 | 10.9 | 11.1 | 10.9 | 12 |
| I-39 | | | | | 21.3 | 21.5 | 23.4 | 25.4 |
| I-42 | | | | | 21 | 20.1 | 20.8 | 22 |
| ID | 30 nM | 30 nM | 10 nM | 10 nM | 3 nM | 3 nM | 1 nM | 1 nM |
| I-9 | 6.1 | 6.9 | 5.3 | 5.7 | 5 | 5 | 4.8 | 4.7 |
| I-17 | 8.4 | 8.8 | 7 | 7.7 | 6.4 | 7.1 | 5.6 | 5.6 |
| I-25 | 7.8 | 7.3 | 7.4 | 6.7 | 5.2 | 5.1 | 5.4 | 5.5 |
| I-26 | 7.6 | 7.5 | 6.6 | 6.8 | 6 | 5.6 | 5.5 | 5.5 |
| I-27 | 9.4 | 10.3 | 7.9 | 8.3 | 8.2 | 7.7 | 5.6 | 5.7 |
| I-28 | 15 | 12.7 | 8.9 | 9.1 | 6.9 | 7.3 | 6.8 | 6.6 |
| I-29 | 8.1 | 8.2 | 7.1 | 7.1 | 6.3 | 6.8 | 6.7 | 6.8 |
| I-30 | 16.3 | 15.3 | 14.1 | 14.1 | 14.7 | 15.7 | 14.3 | 14.7 |
| I-31 | 15.2 | 13.8 | 13.7 | 16 | 13.6 | 14.9 | 14.4 | 13.9 |
| I-32 | 14.7 | 14.3 | 13 | 13.3 | 12.8 | 13.5 | 14.4 | 13.5 |
| I-33 | 28.7 | 28.8 | 23.4 | 22.3 | 15 | 15.3 | 14.5 | 14 |
| I-34 | 17.8 | 16.4 | 14.9 | 14.5 | 13.6 | 13.4 | 13.9 | 15.8 |
| I-35 | 18.3 | 20.2 | 18 | 16.8 | 13.5 | 13.8 | 14.6 | 13.5 |
| I-36 | 23.8 | 24.4 | 20.2 | 20.6 | 13.7 | 14 | 14.7 | 14.5 |
| I-37 | 28.4 | 27.9 | 26.9 | 25.5 | 16.8 | 17.5 | 14.8 | 15.4 |
| I-38 | 10.6 | 11.6 | 11.2 | 10.8 | 10.2 | 10.7 | 10.4 | 10.9 |
| I-39 | 22.7 | 22.2 | 22.4 | 21.4 | 17.7 | 15.9 | 15.7 | 16.5 |
| I-42 | 20 | 19.8 | 17.5 | 18.1 | 15.5 | 14.5 | 13 | 12.4 |
| ID | 0.3 nM | 0.3 nM | 0.03 nM | 0.03 nM | | | | |
| I-9 | 5.1 | 5.5 | | | | | | |
| I-17 | 5.8 | 5.1 | | | | | | |
| I-25 | 5.1 | 5.1 | | | | | | |
| I-26 | 5.1 | 5.1 | | | | | | |
| I-27 | 5 | 5.2 | | | | | | |
| I-28 | | | | | | | | |
| I-29 | 6.7 | 6.9 | | | | | | |
| I-30 | | | | | | | | |
| I-31 | | | | | | | | |
| I-32 | | | | | | | | |
| I-33 | | | | | | | | |
| I-34 | | | | | | | | |
| I-35 | | | | | | | | |
| I-36 | | | | | | | | |
| I-37 | | | | | | | | |
| I-38 | | | | | | | | |
| I-39 | 11.9 | 12.1 | 11 | 11 | | | | |
| I-42 | 11 | 11.4 | 10.3 | 11.3 | | | | |

EC50 (nM) from Certain Assessments as Examples:

| ID | EC50 (nM) |
|---|---|
| I-9 | 27 |
| I-17 | 5.9 |
| I-25 | 15 |
| I-26 | 9.5 |
| I-27 | 2.4 |
| I-28 | 18 |
| I-29 | 62 |
| I-30 | 175 |
| I-31 | >3000 |
| I-32 | 93 |
| I-33 | 11 |
| I-34 | 23 |
| I-35 | 9.3 |
| I-36 | 9.8 |
| I-37 | 5.4 |
| I-38 | >3000 |
| I-39 | 3 |
| I-40 | 1.9 |
| I-42 | 5 |
| I-41 | 50 |
| I-43 | ND |
| I-44 | ND |

### Example 5. Provided Compounds do not Deplete CD38-Expressing Effector Cells

In some embodiments, provided technologies provides various advantages compared to existing CD38-based technologies, e.g., those using CD38 antibodies. Among other things, provided technologies do not deplete or significantly reduce normal CD38-expressing cells such as many immune cells compared to CD38 antibody based technologies. As appreciated by those skilled in the art, various technologies can be utilized to assess depleting of CD38-expressing effector cells. In one example, NK cells were purified from frozen stock using 3×10\hat{ }7 PBMC preparations (STEMCELL technologies) using EasySep™ Human NK cell Isolation Kit STEMCELL Technologies (cat #17955) according to manufacturer's instructions. NK cells were plated at the density of 2×10\hat{ }6 cells per mL of OPTIMEM media, in a 100 uL volume per well of round bottom 96 well plates. Daratumumab was added to a final concentration range of 3 ug/mL to 0.1 ug/mL. Compound I-9 was added to the culture at the final concentration range of 300 nM to 10 nM. IvIG was used at a final concentration of 10 ug/mL. Cells were mixed by pipetting up and down and incubated for 18 h at 37° C. At the end of the incubation period, cells were centrifuged at 400 g for 5 min and re-suspended in PBS. Following this, cells were stained with 1 uM Zombie viability dye (Biolegend, catalog #423111) according to manufacturer's instructions and incubated for 15 minutes at room temperature. Fluorescent antibody cocktail was then added to the cells which included CD56 PE, CD38 PE-Cy7, CD3 BV786, and CD107a BV421) in PBS 1% BSA, 0.5 mM EDTA buffer. Cells were further incubated for 15 min at 4 C.°, and washed twice with 200 uL of PBS 1% BSA, 0.5 mM EDTA buffer. Cells were re-suspended in a final volume of 150 uL of PBS 1% BSA, 0.5 mM EDTA buffer, and 20 uL of Count Bright™ Absolute Counting Beads (Thermofisher Catalog #C36950) to determine absolute cell counts. Cells were analyzed on BD FACSCelesta flow cytometer (BD Biosciences). NK cells were defined as CD3⁻CD56⁺. Percentages of Zombie green positive NK cells were analyzed and graphed using GraphPad Prism software. Data from one example were presented in FIG. 4. As demonstrated, provided ARMs did not deplete CD38-expressing effector cells, e.g., NK cells. Various additional assays including in vivo animal models demonstrated that provided technologies can effectively kill diseased cells such as cancer cells, did not induce NK cell fratricide or CDC, and/or did not significantly impact immune cell populations (e.g., various types of immune cells, e.g., in bone marrow immune cell populations) compared to CD38 antibodies such as daratumumab, confirming that provided technologies can be highly effective without causing significant side effects/toxicity compared to antibody-based technologies.

### Example 6. Provided Technologies can Effectively Kill Cancer Cells without Causing Significant Side Effects

Cancer therapies can have various side effects. For example, those utilizing antibodies toward certain cancer antigens may harm, inhibit or kill non-cancer cells comprising the same antigens. Among other things, the present disclosure present results, below, confirming that provided technologies, while being effective toward cancer cells expressing various antigens, have much less side effects/toxicity compared to other technologies including antibody-based technologies targeting the same antigen. For example, compared to other technologies, provided technologies do not significantly reduce number of non-cancer cells expressing the same targets.

Certain Useful Procedures

PBMC isolation: Leukopheresis product from a donor with body mass index (BMI) between 19-25, under 50 years old, not on immuno-suppressive drugs for at least 2 weeks with an estimated PBMC cell count of >10\hat{ }10 was obtained from Key Biologics (subject ID 20982200) and transported at ambient temperature. Leukopheresis bag was wiped down with 70% EtOH, a small slit was made and contents were pipetted off into tubes. Leukopak contents were diluted 1:1 with PBS without Ca++/Mg++ and 20 ml of this mixture was layered on top of 25 mL of Ficoll. Tubes were then centrifuged at 400 g for 30 minutes without brakes. PBMCs were harvested from the interface into separate tubes, and tubes were filled to 50 mL with PBS. Cells were centrifuged for 10 min at 120 g to get rid of platelets. Supernatant was poured off and cells were re-suspended in RPMI 10% FBS at a concentration of 7-10×10\hat{ }6 cells/ml. Cells were cultured overnight at 37 C in a 5% CO₂ atmosphere until NK cell isolation.

NK cell isolation: PBMC were centrifuged at 400 g for 10 min and re-suspended in EasySep buffer. NK cells were isolated using NK cell isolation kits from STEMCELL Technologies according to kit's manufacturers' instructions. NK cell purity and phenotype was assessed by trypan Blue and flow cytometry using the following antibody cocktail: anti-CD56PE, CD3 APC, and viability was determined using staining with Trypan Blue exclusion dye, and by flow cytometry using Near Infra-Red Fixable viability dye (Thermo Fisher).

CIML NK cell generation: Isolated NK cells were re-suspended at 2×10\hat{ }6 cells/ml in XVIVO 10% Human Serum and the following concentrations IL-15 (50 ng/ml), IL-12 (10 ng/ml), IL-18 (50 ng/ml) for 12 to 18 hrs. Cells were harvested and tested for purity and viability by Trypan Blue staining and by Flow Cytometry using Near Infra-Red Fixable viability dye (Thermo Fisher), CD3 APC (Biolegend) and CD56 PE (Biolegend).

Treatment with I-17: 1-17 was dissolved in DMSO to make a 25 mM stock solution. The stock was then serially diluted in 100% DMSO to achieve 1000× concentrations to be used in the experiment. The 100% DMSO compound stock was then added to a tube and media was added to a volume of 1000 ul per 1 ul of compound and vortexed for 1 minute to ensure a homogenous mixture. NK cell pellets were directly re-suspended in the I-17 solutions at cell density of 5×10\hat{ }6 cells/mL.

CIML NK cell Fratricide assay: NK cells were directly re-suspended in solutions generated for each concentration of the I-17 at a density of 5×10\hat{ }6 cells per milliliter. Similarly, NK cell pellets were re-suspended in Daratumumab control antibody solutions (a range of 3 ug/ml to 0.01 ug/ml in PBS 5% HSA). Resulting NK cell suspensions were aliquoted at 55 ul/well of a 96 wellv-bottom plate and were incubated for 2 hrs at 37° C. in a 5% CO₂ atmosphere.

CIML NK cell SUDHL-4 cell ADCC assay: SUDHL-4 cells were labelled with CFSE and re-suspended in XVIVO15 media containing 20% Human serum. Cells were aliquoted at 10,000 cells/well, 18 ul/well of 96 well V-bottom plates. NK cells (in PBS 5% HSA) were added at a 9:1 ratio (90,000 NK cell per well) in an equal volume (18 ul per well). Co-cultures were incubated overnight at 37° C. in a 5% CO2 atmosphere. Next day, cells were washed, and stained with the following reagents: Near Infra-Red Fixable viability dye (Thermo Fisher), CD3 FITC (Biolegend) and CD56 PE (Biolegend). Percentages of Dead SUDHL-4 cells were calculated by gating on CFSE+ cells that are also positive for Near Infrared Live Dead dye.

Certain Results

Purity and viability of NK cells and CIML NK cells: Among other things, the present disclosure provides highly pure and viable NK cell populations following isolation from PBMC. Peripheral blood mononuclear cells were isolated form leukopheresis product PBMC viability post-isolation was >99% by Trypan blue and by flow cytometric analysis. After isolation using magnetic bead assisted negative selection, NK cell were 90% pure, and more than 99% viable both by Trypan blue and flow cytometry. T cell (CD3+CD56− cells) contamination in the NK cell fraction was found to be 2.8% on a day of isolation. After NK cells were isolated, they were divided into two treatment groups—CIML NK cells and non-CIML (control cells). CIML NK cells were incubated overnight in XIVO15 media containing 10% human sera and IL-12, IL-15, and IL-18, whereas control, non-CIML NK cells, did not receive the cytokine treatment. After an overnight incubation, purity and viability were assessed in the cultured cell populations. In one experiment, CIML and non-CIML control NK cells were 50% viable by Trypan Blue. CIML NK cells were 75% viable by Flow Cytometry, whereas viability of control non-CIML NK cells by flow cytometry method was at 78%. At the time of harvest, T cell contamination in both cultures was 1.2-1.8%.

Provided Technologies Demonstrated Significantly Low Toxicity Compared to Corresponding Technologies Comprising Antibody Toward the Same Targets.

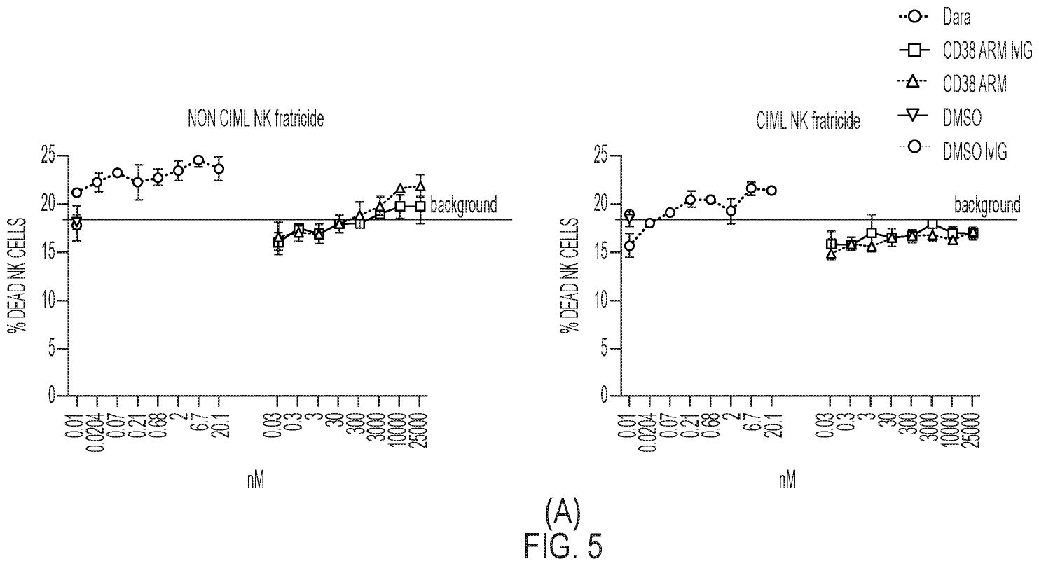

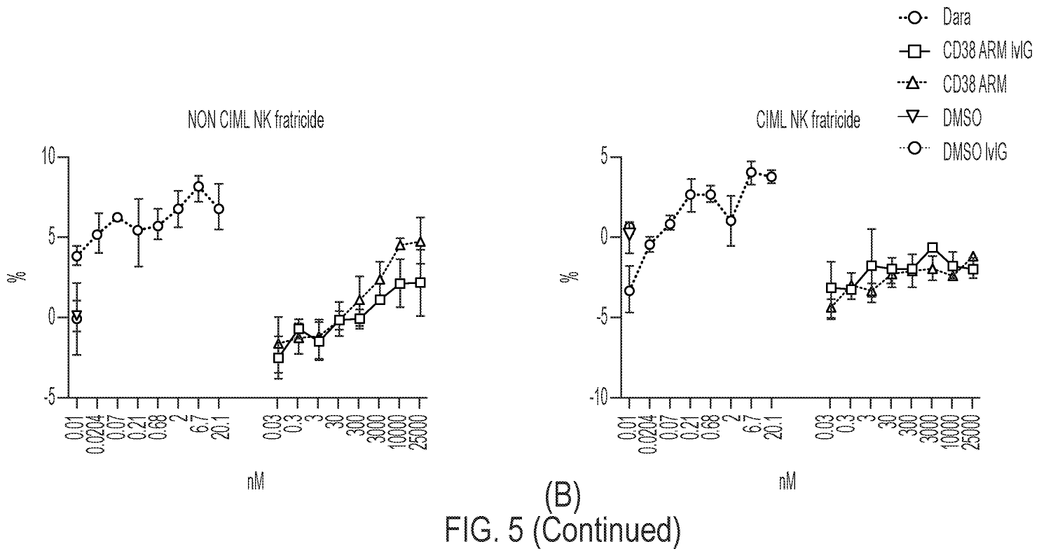

NK cell fratricide (death directed at NK cells in the same culture) was assessed by flow cytometry following a 2 hr incubation with the I-17 at indicated concentrations in the presence or absence of 500 ug/ml intravenous immune globulin (IVIG). FIG. 5 illustrates frequencies of dead NK cells as a function of daratumumab, a CD38 directed therapeutic antibody, or the I-17 concentrations in culture supernatants.

As demonstrated, provided technologies shown significantly less toxicity than corresponding technologies using antibodies, e.g., when assessed by reduction of cell number of non-cancer cells. For example, at just 2 hours post-incubation, for CIML NK cells, daratumumab treatment at 3 ug/ml or 20.1 nM, resulted in a 4% increase in the percentage of dead NK cells present in the cultured media compared to no increase with the I-17+/−IVIG, FIG. 5. In a culture of non-CIML NK cells daratumumab treatment produced a total increase in dead NK cells of 5.6% over DMSO treated control, whereas treatment with the highest concentration of the I-17 (25 uM) resulted in a 1.7% increase (the I-17 at 25 uM vs DMSO control), and 0.93% increase in dead NK cells (the I-17 IvIG vs DMSO IVIG control). FIG. 5 (B) represents data normalized to a DMSO treated control. As shown, no apparent NK cell fratricide was detected with even the highest doses of the I-17, in sharp contrast to daratumumab, a CD38 therapeutic antibody approved by FDA for treatment of multiple myeloma.

Provided Technologies can Effectively Reduce Numbers of Target Cancer Cells.

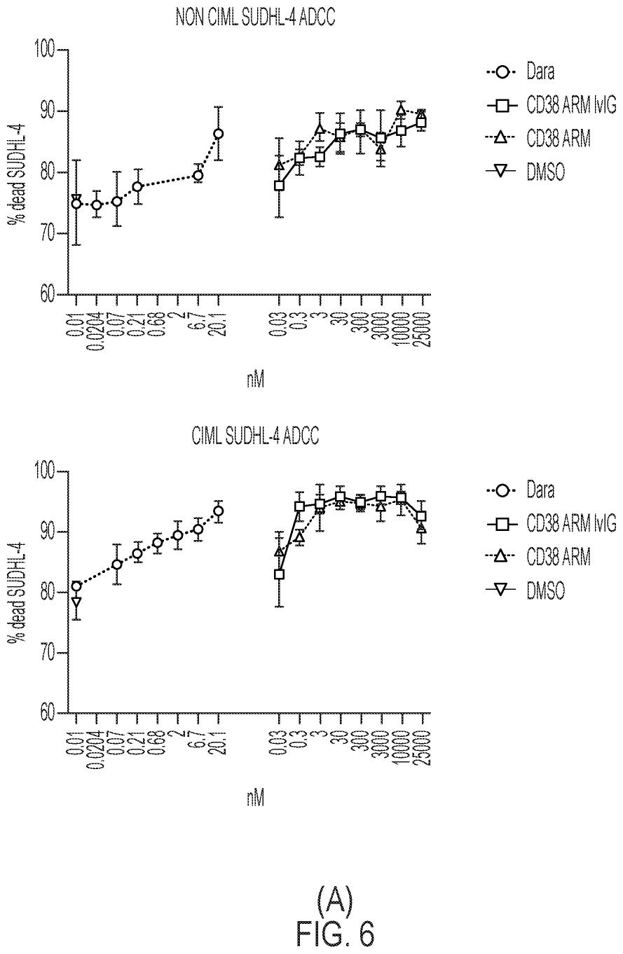

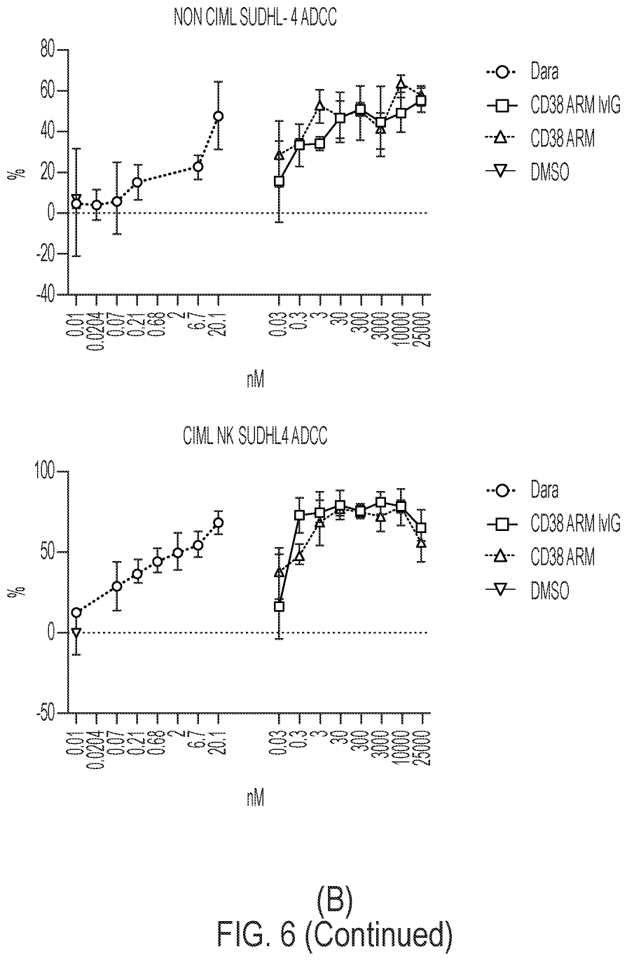

Antibody dependent cell cytotoxicity assay was performed to measure the effects of CIML NK cells alone and in combination with the I-17 on target SUDHL-4 multiple myeloma cells. Baseline activity was assessed by using NK cells that have not been stimulated with cytokine cocktail (non-CIML NK cells). FIG. 6 represents frequencies of dead SUDHL-4 cells in NK-SUDHL-4 cell co-cultures after 18 hours of incubation.

As demonstrated from data presented in FIG. 6B, CIML NK cells are potent killers of SUDHL-4 target cells at the baseline (DMSO treated controls). Additional target cell killing over DMSO-treated control level was achieved by CIML NK cell in combination with the I-17—a 66% increase in killing over background was observed. Further, as demonstrated herein provided technologies can effectively utilize IgG present in human serum and do not require administration of additional antibodies, e.g. exogenous antibodies like those utilized in antibody-based therapies. Data using CD38-targeting daratumumab were also included. As demonstrated herein, provided technologies are highly effective in killing cancer cells while being less toxic compared to antibody-based technologies.

### Example 7. Provided Technologies can Effectively Kill Intended Cells with Low Toxicity

Among other things, the present example confirms that provided technologies can effectively reduce the number of cancer cells. In the present example, I-17 (as in the example above), cytokine induced memory like NK cells (CIML NK cells), and optionally intravenous immunoglobulin (IVIG) were utilized. Among other things, the present disclosure confirms activity of provided technologies against multiple myeloma patient bone marrow plasma cells.

Materials, Methods and Equipment

Test and Control Articles

I-17 (25 mM solution in DMSO)

Daratumumab (Darzalex)—Parexel DRE0607

IVIG Human Immune Globulin Intravenous, Flebogamma—Grifols NDC 61935-0005-5

Materials and Equipment
Bovine Serum Albumin (BSA) Heat Shock, Fraction V—American Bio AB01088-00100
Phosphate Buffered Saline (PBS)—Gibco 10010, (−) Ca (−) Mg, pH 7.4
IVIG Human Immune Globulin Intravenous, Flebogamma—Grifols NDC 61935-0005-5, 100 mg/ml—Dilute to 1 mg/ml in PBS
Biotinylated/His tagged Human CD38 Protein, Avi Tag—Acro Biosystems CD8-H82E7
Neutravidin—Invitrogen 31000, 1 mg/ml
Dimethyl Sulfoxide (DMSO) Sigma-Aldrich 276855
Bovine Serum Albumin (BSA) Heat Shock, Fraction V—American Bio AB01088-00100
Ethylenediaminetetraacetic acid (EDTA) VWR 97062-656
Flow buffer—PBS pH 7.4+0.05% (w/v) BSA, 2 mM EDTA
Assay Plate, 96-well V-Bottom—VWR Corning 3357
Attune NxT Flow Cytometer and Attune NxT Software
96 well 2 ml deep well plate USA Scientific, Cat #1896-2110
Ficoll® Paque Plus, GE Healthcare, Cat #17-1440-03
EasySEP™ Human NK cell isolation kit STEMCELL Technologies Cat #17955
Human IL-15 premium grade Miltenyi Biotec Cat #130-095-764
Human IL-12 premium grade Miltenyi Biotec Cat 130-096-705
Human IL-18 R&D Systems Cat #9124-IL-010
Specialty Cell Culture Medium, Lonza X-VIVO15, Thermo Fischer Scientific Cat#BW04-744Q
Human Serum AB off the Clot Gemini Bioproducts, Cat #100-318
Albumin from human serum, Sigma Aldrich, Cat#A5843-5G
RBC lysis buffer 10× (Tonbo Biosciences Cat#TNB-4300-L100

Minimal Residual Disease Antibody Staining Panel:

| Antigen | Fluorophore | Clone | Vendor | Catalog # |
|---|---|---|---|---|
| CD138 | BV421 | MI15 | BD Biosciences | 562935 |
| CD27 | BV510 | O323 | Biolegend | 302835 |
| CD38 ME | FITC | multiepitope | Cytognos | CYT-38F2 |
| CD56 | PE | 5.1H111 | Biolegend | 362508 |
| CD45 | PERCPCY5.5 | HI30 | Biolegend | 304028 |
| CD19 | PECY7 | HIB19 | Biolegend | 302216 |
| CD117 | APC | 104D2 | BD | 341096 |

Patient bone marrow and blood were obtained from Discovery Life Sciences.

Protocols

Patient bone marrow (3 ml) and blood (20 ml) were shipped at ambient temperature from Discovery Biosciences. Bone marrow was diluted 1:1 with PBS, and 50 Dl of bone marrow-PBS mixture was aliquoted into 96 deep well plates. Bone marrow cells were incubated overnight until CIML cells were added next day.

PBMC were isolated from blood by Ficoll density gradient centrifugation. PBMC were centrifuged at 400 g for 10 min and re-suspended in EasySep buffer. NK cells were isolated using NK cell isolation kits from STEMCELL Technologies according to kit's manufacturers' instructions.

NK cells were re-suspended at 1×10⁶ cells/ml in XVIVO15, 10% Human Serum, IL-15 (50 ng/ml), IL-12 (10 ng/ml), IL-18 (50 ng/ml), and incubated for 18 hrs. Following overnight incubation with cytokines, NK cells were washed twice with PBS 5% Human Serum Albumin, counted and re-suspended at a cell density of 2×10⁵ cells/ml of XVIVO15 media. Twenty microliters (4000 cells) were added per each well containing 50 □l of bone marrow: PBS mixture. Daratumumab (3 □g/ml [20 nM] to 0.3 □g/ml [2 nM]) and the I-17 (25□M to 3 □M final concentration) were made up to a 10× of the final concentration to be used in the assay and 8 □l of the I-17 or 7 □l of Daratumumab 10× stock solutions were added to pertinent wells. To the I-17 treated wells, human IVIG was added to a final concentration of 10 Dg/ml. Control wells received XVIVO15 media. Well contents were mixed by pipetting up and down and plates were centrifuged at 400 g for 1 min.

The assay was then incubated for 4 hrs at 37° C. 5% CO₂. Following the incubation, 1 ml of 1× red blood cell lysis buffer was added to each well and cells were incubated for 15 min at room temperature. Plates were centrifuged at 400 g for 3 minutes, and another round red blood cell lysis and centrifugation was performed. Cells were then re-suspended 200 □l of FACS buffer and centrifuged. Buffer was discarded, and cells were re-suspended in a 100 □l of fluorescent antibody mixture. Cells were incubated for 15 min at 4° C., to wash 200 □l of FACS Buffer was added per each well, cells were centrifuged at 400 g for 3 min, and supernatants were discarded. Washing was repeated 2 more times. Finally, cells were re-suspended in 200 □l of FACS Buffer and analyzed using Attune Flow Cytometer. Data were analyzed using FlowJo software and graphed using GraphPad Prism.

Provided Technologies Reduce Number of Plasma Cells.

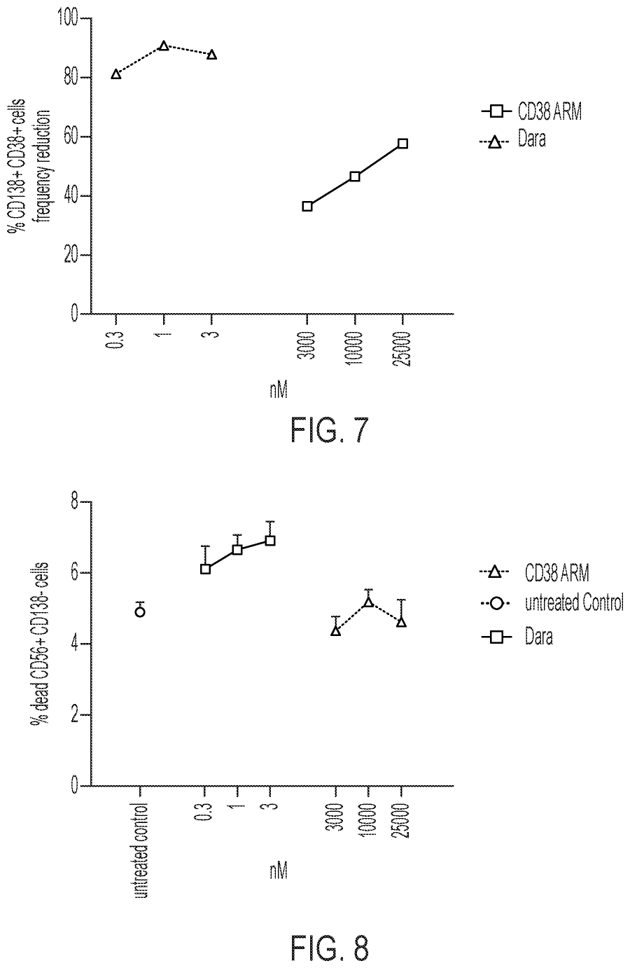

FIG. 7 shows the percent reduction in the frequencies of CD38+ CD138+ cells in patient bone marrow after a 4 hr incubation in the presence of autologous patient CIML NK cells and daratumumab or I-17. As demonstrated in FIG. 7, I-17 effectively reduced frequency of plasma cells (CD38+ CD138+) compared to control untreated bone marrow incubated with CIML NK cells.

Provided Technologies do not Significantly Impact Immune Cell Populations.

Among other things, the present example further confirms that provided technologies are of low toxicity, particularly when compared to therapies utilizing antibodies toward the same targets as ARMs. As shown in FIG. 8 (frequencies of CD56+CD138-CIML NK cells when co-incubated with patients bone marrow and Daratumumab or I-17 for 4 hrs.), NK cell numbers did not change in the presence of I-17, while daratumumab showed a dose-dependent increase in NK cell fratricide.

### Example 8. Provided Technologies can Effectively Reduce Number of Target Cells

As described herein, provided technologies can effectively reduce number of target cells, e.g., cells expressing CD38. Without the intention to be limited by theory, in some embodiments, reduction of target cell numbers may comprise or be through ADCP, in some instances mediated through macrophage. Described below are useful technologies for assessing provided compounds, compositions and methods, and illustrative data confirming activities of provided technologies (e.g., compounds, compositions, methods, etc.).

Materials and Methods

Test System

| | |
|---|---|
| Species/strain: | Mouse/SCID |
| Physiological state: | Normal |
| Age/weight range at start of study: | Animals aged ~6 weeks with body weight of approximately 25 g. |
| Animal supplier: | Charles River Laboratory |
| Number/sex of animals: | 32/Female |
| Identification: | Ear notch/transponder |
| Randomization: | Animals will be randomized prior to assignment to treatment |
| | groups. |
| Justification: | The mouse represents a well characterized system for drug |
| | efficacy evaluation. |
| Replacement: | Animals will not be replaced during the course of the study. |

Experimental Design

On day 0 animals were weighed and randomized. Mice were assigned to different groups and treated as described below. CFSE labelled Daudi cells were prepared as follows:

### CFSE Staining of Daudi Cells for IP Injection:

Reagents used: PBS (Sterile)
Quenching Buffer PBS+10% FBS
RPMI (no additives) no P/S; no FBS
Invitrogen—CellTrace CFSE Cell Proliferation Kit, for Flow Cytometry (Cat#C34570)
Warm the sterile PBS and all other media to 37 degree Celsius before use for all of the following procedures listed below.
   Harvest enough cells from cell culture to include 50% more cells than required for assay
Wash the cells 2× with sterile PBS by centrifuging @ 500 g for 7 minutes.
Resuspend cells in sterile PBS to prepare a cell suspension in a 50 mL polypropylene tube such that the cell suspension occupies less than ¼ the total volume of the tube.
Prepare a 5 mM stock solution of CFSE by adding 18 uL of DMSO to the lyophilized tube of CFSE.
   From stock, dilute in PBS 10-fold to make 100× (500 uM) solution
   Add 10 uL or 100×CFSE solution per each 1 mL of cells
Add CFSE stock solution to the resuspended cells so that the final concentration of the CFSE is 5 uM.
Gently mix the cell suspension by swirling till the dense settled DMSO (CFSE) is no longer visible.
Loosen the cap of the 50 mL conical tube (containing CFSE-labeled cells), stand it upright in tube rack, & place in a 37° C. CO₂ incubator for 10 minutes while swirling every 2 minutes.
After incubation, top off the tube with Quenching Buffer to quench and let sit for 2 min.
Wash the cells 2× extra time with sterile Quenching Buffer by centrifuging @ 500 g for 7 minutes.
Wash the cells 3× with cold sterile RPMI (no additives) by centrifuging @ 500 g for 7 minutes.
   Volume up to 50 mL for each wash step
Perform last cell count:
   Dilute cells to 1:50 in counting vial
Resuspend with approximate vol. cold RPMI (no additives) to desired cell density:
Adjust cell density
   cells will be injected per mouse: 400 uL/mouse; total 20×10⁶/mouse

After 16-18 hours the mice were sacrificed via CO₂ asphyxiation and had their peritoneal cavity flushed with 5 mls of PBS with 1% BSA. Samples were processed and analyzed using IP Lavage Processing is described in 00765. Samples were evaluated for tumor cell counts.

| Group | Number of | CFSE Daudi | IVIG (10 mg SQ) | Treatment |
|---|---|---|---|---|
| Number | Animals | cells (IP) | Day 0 | Day 1 |
| 1 | 4 | 20 × 10⁶ | — | — |
| 2 | 5 | 20 × 10⁶ | X | — |
| 3 | 6 | 20 × 10⁶ | X | I-17 1 mg/kg SQ |
| 4 | 6 | 20 × 10⁶ | X | I-17 10 mg/kg SQ |
| 5 | 6 | 20 × 10⁶ | X | I-17 30 mg/kg SQ |
| 6 | 6 | 20 × 10⁶ | — | Daratumumab 10 mg/kg SQ |

Study Timeline:

| Event | Day 0 | Day 1 | Day 2 |
|---|---|---|---|
| Weight/Randomize | X | | |
| Dose IVIG 10 mg SQ (Groups 3, 4, 5) | X | | |
| Dose I-17 SQ | | X | |
| Dose 20 × 10⁶ Daudi cells (IP) | | X | |
| Daratumumab 10 mg/kg SQ | | X | |
| IP Lavage | | | X |

Animal body weights were measured and recorded at randomization on Day 0. An additional body weight were collected on the same compound administration.

Tissue Harvesting. After 16-18 hours the mice were sacrificed via CO₂ asphyxiation and had their peritoneal cavity flushed with 5 mL of PBS with 1% BSA. Samples were kept on ice until further processed.

A useful IP Lavage Processing protocol:
1. Intraperitoneal lavage fluid is harvested using 5 ml of PBS 1% BSA without IgG.
2. Spin down lavage samples for 5 min at 400 g.
3. Maintain labelling of tubes as originally received from animal facility and keep samples on ice.
4. Record the volume of each sample on a spreadsheet.
5. Pour off supernatants to 5 ml Eppendorf tubes and store at -80.
6. Pellets are re-suspended in 250 uL of PBS 1% BSA without IgG and transferred to blue top filter flow tubes.
7. Spin filter top tubes down at 400 g for 5 min.
8. Pour off supernatant into a waste tube.
9. RBC present? Proceed with below steps:
   a. RBC lyse by re-suspending pellets in 1 ml RBC lysis buffer 5 min at RT, add 1 ml of PBS 1% BSA buffer to each tube and spin 5 min at 400 g.
   b. Re-suspend the pellets by pipetting up and down in 250 uL of PBS 1% BSA without IgG and transfer the entire cell suspension into 96 well plates marked with sample numbers.
   c. Spin the plate down at 400 g for 3 min and flick away supernatant, plate on ice.

A useful protocol for preparing for flow analysis on Attune:
1. Re-suspend the cells in 250 uL in PBS 1% BSA without IgG in tube.
2. Split samples from above step into 2 identical 96-well V-bottom plates.
3. Fixing Procedure:
   a. Fix at 4% Paraformaldehyde (Stock 16%—Dilute 1:4 with PBS).
   b. Spin plates at 400 g for 5 minutes and flick away supernatant.
   c. Resuspend with 125 ul PBS 1% BSA without IgG.
   d. Add and mix gently with pipette 125 ul 4% Paraformaldehyde.
   e. Incubate at room temperature for 10 minutes.
   f. Spin plates at 400 g for 5 minutes and flick away supernatant.
   g. Resuspend in 250 ul PBS 1% BSA without IgG.
   h. Store at 4 degrees Celsius.
4. Running Fixed samples:
   a. Remove plate from fridge.
   b. Spin plates at 400 g for 5 minutes and flick away supernatant.
   c. While plate is spinning, run CST beads on Attune and set up experiment.
   d. Resuspend with 250 ul PBS 1% BSA without IgG.

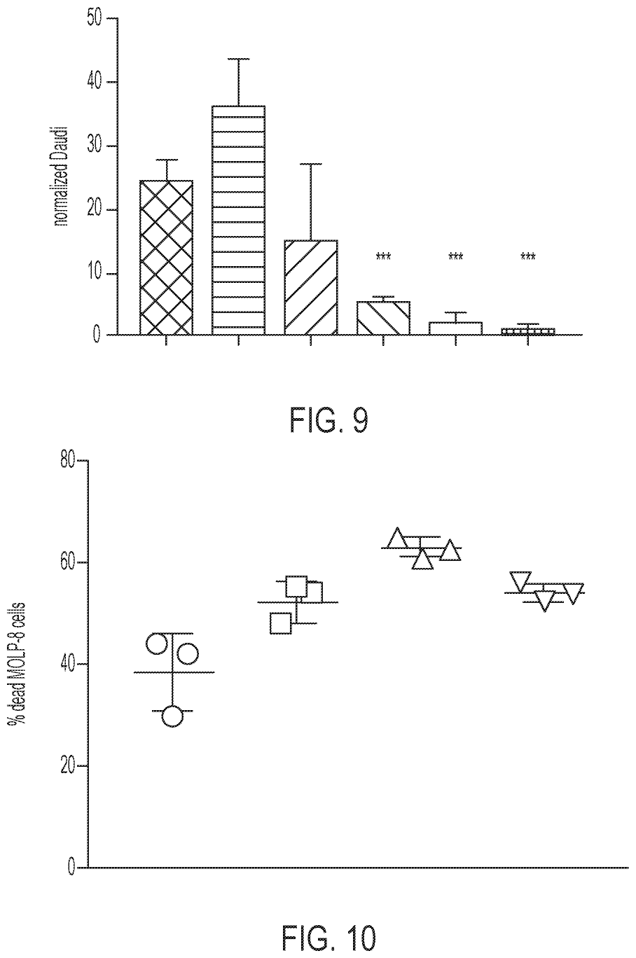

Certain data were presented in FIG. 9. As confirmed herein, provided technologies can effectively reduce number of target cells as demonstrated in the dose-dependent reduction of Daudi cells in intraperitoneal lavage model, which, without the intention to be limited by theory, may be through or comprise macrophage-mediated does dependent ADCP of Daudi cells.

### Example 9. Compositions Comprising Cells can be Cryopreserved

Among other things, the present disclosure demonstrates that compositions comprising cells can be cryopreserved and utilized later to provide desired activities. In an example:

1. CIML NK cells were generated from same day arrival leukopack and had viability of greater than 95% before freezing.
2. PBMC, NK cell isolation, and CIML generation as described (e.g., Example 6; Romee et al. Science Translational Medicine 21 Sep. 2016: Vol. 8, Issue 357, pp. 357ra123; DOI: 10.1126/scitranslmed.aaf2341).
3. CIML NK cells where washed from cytokines with RPMI 10% FBS twice.
4. Assessed viability prior to freezing by Trypan Blue or a L/d discrimination dye such as L/D near IR.
5. Cells were frozen in FBS 10% DMSO at 100×10\hat{ }6 cells per ml per vial, or in the same media+25 uM I-17:
Briefly, 25 mM stock of I-17 was generated in DMSO.
This was diluted further in DMSO 1:100 to make 250 uM.
Freezing media was made:

Without I-17 90% FBS+10% DMSO—for control cells.

With I-17: 90% FBS+10% 250 uM I-17 solution in DMSO—for cells frozen with I-17.

6. Cells were put in mister Frosty isopropanol container and stored at -80 C.
7. Cells were kept at -80 for 1 day, then switched to liquid nitrogen.
8. Cells were in liquid nitrogen for 2 weeks before thaw.
9. Thawed vial in 37 H2O bath for more than 5 min.
10. Added dropwise to 35 mL of warm RPMI 10% FBS.

### 11. Centrifuged for 10 min at 400 g.

12. Washed 1× with RPMI 10% FBS.

### 13. Centrifuged for 10 min at 400 g.

14. Counted and assessed viability using near IR live dead viability dye or similar.
15. Proceed to ADCC: MOLP8 cells labelled with CFSE are re-suspended at 10 k/50 ul of RPMI 10% FCS.
16. Added to round-bottom 96 well plates.
17. NK cells were re-suspended at 100K/50 uL, 50K, and 25K and added to target cells.
18. To CIML cell conditions added daratumumab at 3 ug/mL or I-17 at 2.5 uM+10 ug/mL IvIG.
19. To wells that have received CIML NK cells frozen in combo with I-17, only add IvIG.

### 20. Incubated for 4 hrs.

21. Stained with IR L/D, CD107a, and CD69.

As demonstrated in FIG. 10, compositions comprising cells can be cryopreserved and then thawed and utilized with provided compounds (in the same cryopreserved composition and/or added separately) to provide activity. In some embodiments, cells are preserved with provide compounds, e.g., ARM agents. In some embodiments, cells are preserved without provided ARM agents, which can be added separately.

While we have described a number of embodiments, it is apparent that our basic examples may be altered to provide other embodiments that utilize compounds and methods of the present disclosure. Therefore, it will be appreciated that the scope of an invention is to be defined by claims rather than by the specific embodiments that have been represented by way of example.

1. (canceled)

2. (canceled)

3. An agent that has the structure: 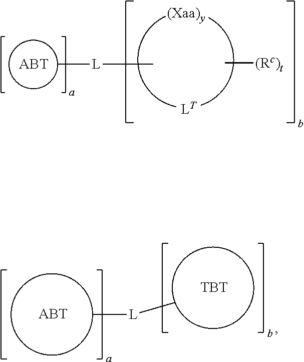
or a pharmaceutically acceptable salt thereof,
wherein:
each of a and b is independently an integer from 1 to 200;
each ABT is independently an antibody binding moiety;
L is a bivalent linker moiety that connects ABT with TBT;
each TBT is independently a target binding moiety that has the structure 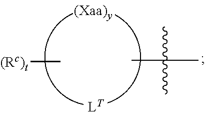
each Xaa is independently a residue of an amino acid or an amino acid analog;
y is an integer from 5 to 20;
L^{T} is a linker moiety linking two residues each independently of an amino acid or an amino acid analog, and is independently a covalent bond, or an optionally substituted bivalent group selected from C₁-C₆ aliphatic or C₁-C₆ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each R^{c} is independently -L^{a}-R′;
t is an integer from 2 to 50;
each L^{a} is independently a covalent bond, or an optionally substituted bivalent group selected from C₁-C₅₀ aliphatic or C₁-C₅₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

4. (canceled)

5. The agent of claim 3, wherein -(Xaa)y- comprises:
-Xaa^{T1}-Xaa^{T2}-(Xaa)y′-Xaa^{T3}-Xaa^{T4}-Xaa^{T5}-,
wherein:
y′ is an integer from 0 to 8;
Xaa^{T1} is a residue of an amino acid or an amino acid analog whose side chain is substituted C₁-C₈ aliphatic;
Xaa^{T2} is a residue of an amino acid or an amino acid analog whose side chain comprises an optionally substituted aromatic group or is optionally substituted C₃-C₈ aliphatic;
Xaa^{T3} is a residue of an amino acid or an amino acid analog whose side chain is optionally substituted C₂-C₈ aliphatic;
Xaa^{T4} is a residue of an amino acid or an amino acid analog whose side chain comprises an optionally substituted aromatic group, or is optionally substituted C₃-C₈ aliphatic; and
Xaa^{T5} is a residue of an amino acid or an amino acid analog whose side chain is substituted C₁-C₈ aliphatic.

6. The agent of claim 5, wherein:
Xaa^{T1} is a residue of Ahp, Y, W, S, K, or K(MePEG4c);
Xaa^{T2} is a residue of Y, W, Ahp, Bph, L, or A;
Xaa^{T3} is a residue of L, Ahp, V, T, Hse, or MetO2;
Xaa^{T4} is a residue of Bph, V, or Ahp;
Xaa^{T5} is a residue of Ahp, Bph, Ado, Ano, PhNle, or PhNva; or
a combination thereof.

7. (canceled)

8. (canceled)

9. (canceled)

10. (canceled)

11. The agent of claim 6, wherein -(Xaa)y- is or comprises:
-(Xaa)ₐ₁-(Xaa)ₐ₂-(Xaa)ₐ₃-(Xaa)ₐ₄-(Xaa)ₐ₅-(Xaa)ₐ₆-(Xaa)ₐ₇-(Xaa)ₐ₈-(Xaa)ₐ₉-(Xaa)ₐ₁₀-(Xaa)ₐ₁₁-(Xaa)ₐ₁₂-(Xaa)ₐ₁₃-,
wherein:
each of a1, a2, a3, a4, a5, a6, a7, a8, a9, a10, a11, a12 and a13 is independently 0-5;
(Xaa)ₐ₃ is or comprises Xaa^{T1};
(Xaa)ₐ₄ is or comprises Xaa^{T2};
(Xaa)ₐ₉ is or comprises Xaa^{T3};
(Xaa)ₐ₁₀ is or comprises Xaa^{T4}; and
(Xaa)ₐ₁₁ is or comprises Xaa^{T5}.

12. The agent of claim 11, wherein:
(Xaa)ₐ₁ is or comprises A, K or K(MePEG4c);
(Xaa)ₐ₂ is or comprises R, S, D, Y, A, W, K, 4Py2NH2, Cit, F3G, hCit, K(MePEG4c), RNdMe, RNMe, or RNNdMe;
(Xaa)ₐ₅ is or comprises H, Y, S, L, A, W, or W6N;
(Xaa)ₐ₆ is or comprises D, G, R, Y, H, W, A, or Y;
(Xaa)ₐ₇ is or comprises G, D, E, O, N, R, MetO2, S, Har, or A;
(Xaa)ₐ₈ is or comprises V, A, D, G, W, S, or T;
(Xaa)ₐ₁₂ is or comprises of D, A, S, G, or Ahp;
(Xaa)ₐ₁₃ is or comprises C; or
a combination thereof.

13. (canceled)

14. (canceled)

15. (canceled)

16. (canceled)

17. (canceled)

18. (canceled)

19. (canceled)

20. The agent of claim 3, wherein -(Xaa)y- comprises:
-Xaa^{T6}-(Xaa)y′-Xaa^{T7}-Xaa^{T8}-Xaa^{T9}-Xaa^{T10}-Xaa^{T11}-,
wherein:
y′ is an integer from 0 to 8;
Xaa^{T6} is a residue of an amino acid or an amino acid analog whose side chain is substituted C₁-C₈ aliphatic;
Xaa^{T7} is a residue of an amino acid or an amino acid analog whose side chain is optionally substituted C₂-C₈ aliphatic;
Xaa^{T8} is a residue of proline or an amino acid analog thereof;
Xaa^{T9} is a residue of an amino acid or an amino acid analog whose side chain comprises an optionally substituted aromatic group, or is optionally substituted C₁-C₈ aliphatic;
Xaa^{T10} is a residue of an amino acid or an amino acid analog whose side chain is substituted C₁-C₈ aliphatic, or an amino acid whose amino group is substituted; and
Xaa^{T11} is a residue of an amino acid or an amino acid analog whose side chain comprises an optionally substituted aromatic group, or is optionally substituted C₁-C₈ aliphatic.

21. The agent of claim 20, wherein:
Xaa^{T6} is a residue of MeF, L, or S;
Xaa^{T7} is a residue of L or MeF;
Xaa^{T8} is a residue of P;
Xaa^{T9} is a residue of Bph, D, or S;
Xaa^{T10} is a residue of V or L;
Xaa^{T11} is a residue of W or R; or
a combination thereof.

22. (canceled)

23. (canceled)

24. (canceled)

25. (canceled)

26. (canceled)

27. The agent of claim 21, wherein -(Xaa)y- is or comprises:
-(Xaa)ₐ₁-(Xaa)ₐ₂-(Xaa)ₐ₃-(Xaa)ₐ₄-(Xaa)ₐ₅-(Xaa)ₐ₆-(Xaa)ₐ₇-(Xaa)ₐ₈-(Xaa)ₐ₉-(Xaa)ₐ₁₀-(Xaa)ₐ₁₁-(Xaa)ₐ₁₂
wherein:
each of a1, a2, a3, a4, a5, a6, a7, a8, a9, a10, a11, and a12 is independently 0-5;
(Xaa)ₐ₄ is or comprises Xaa^{T6};
(Xaa)ₐ₆ is or comprises Xaa^{T7};
(Xaa)ₐ₇ is or comprises Xaa^{T8};
(Xaa)ₐ₈ is or comprises Xaa^{T9};
(Xaa)ₐ₉ is or comprises Xaa¹⁰; and
(Xaa)ₐ₁₀ is or comprises Xaa^{T11}.

28. The agent of claim 27, wherein:
(Xaa)ₐ₁ is or comprises A;
(Xaa)ₐ₂ is or comprises L, A or P;
(Xaa)ₐ₃ is or comprises H, R, or A;
(Xaa)ₐ₅ is or comprises V, A or MeG;
(Xaa)ₐ₁₁ is or comprises V, A, D or MeG;
(Xaa)ₐ₁₂ is or comprises C; or
a combination thereof.

29. (canceled)

30. (canceled)

31. (canceled)

32. (canceled)

33. (canceled)

34. (canceled)

35. The agent of claim 3, wherein:
two Xaa are linked together;
two Xaa are linked together through a linker having the structure of —C(O)—CH₂—;
two Xaa are linked together through a linker having the structure of —C(O)—CH₂—, wherein —C(O)— is bonded to an amino group of a Xaa; or
two Xaa are linked together through a linker having the structure of —C(O)—CH₂—, wherein —C(O)— is bonded to an amino group of a Xaa and —CH₂— is bonded to —S— in the side chain of a Xaa.

36. (canceled)

37. (canceled)

38. (canceled)

39. The agent of claim 3, wherein 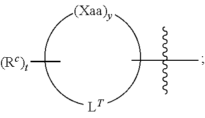
(a) TBT is or comprises a peptide whose sequence is or comprises a sequence selected from SEQ ID NO: 1-34, or
(b) TBT is or comprises 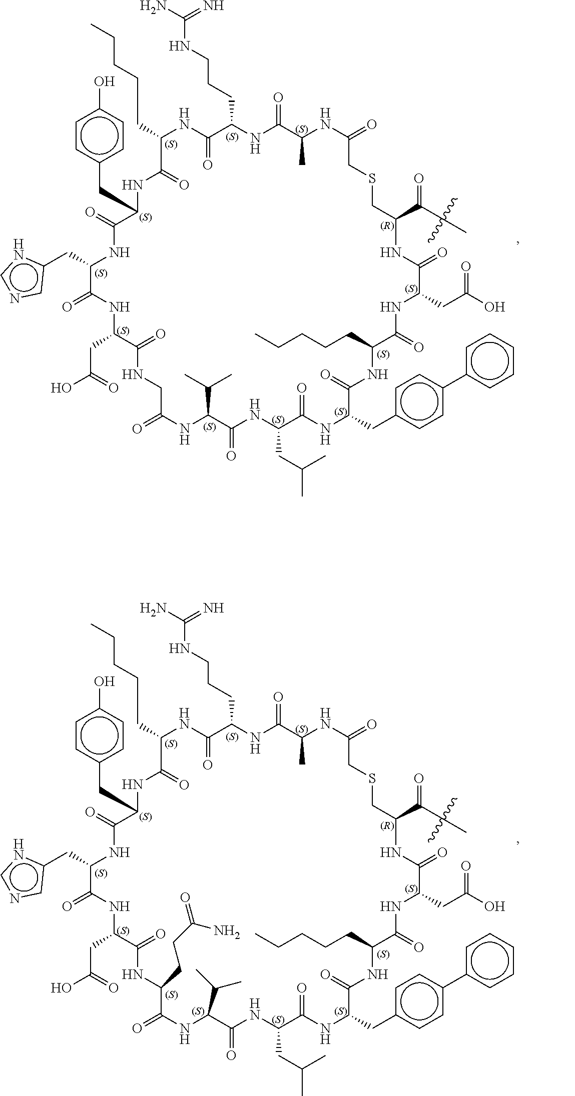 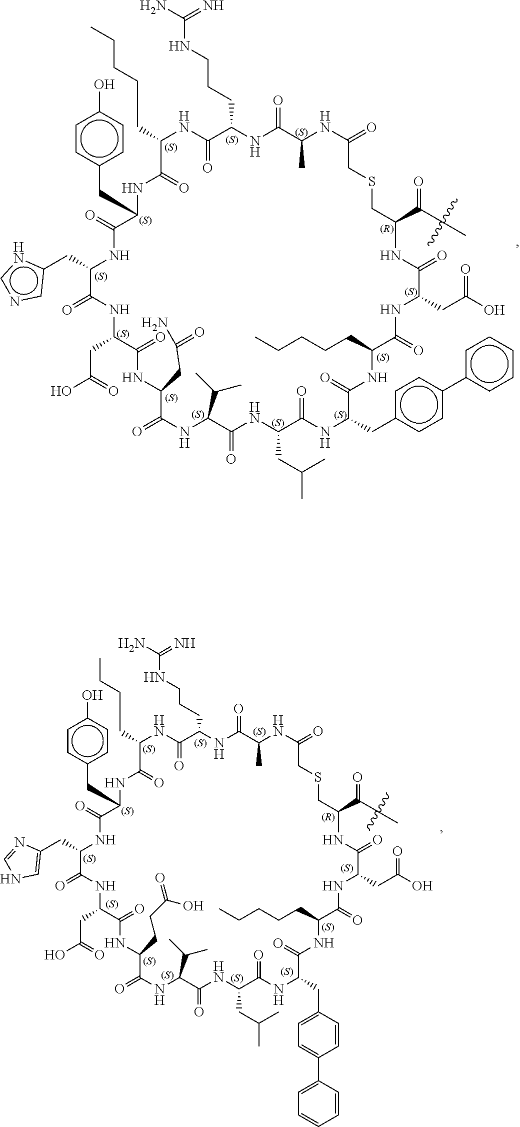 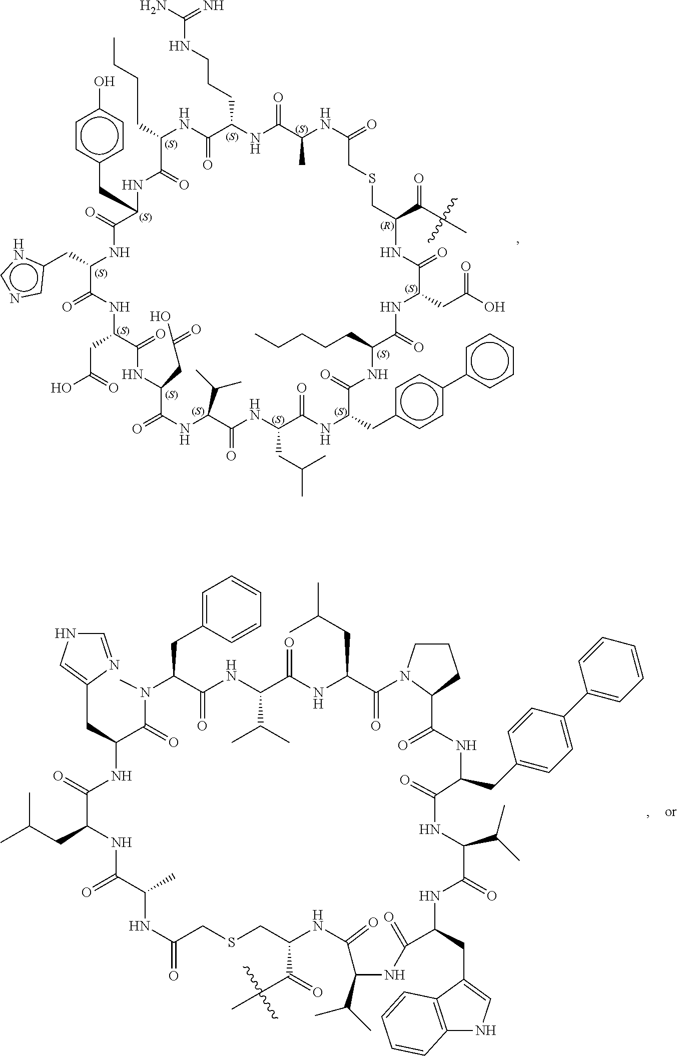 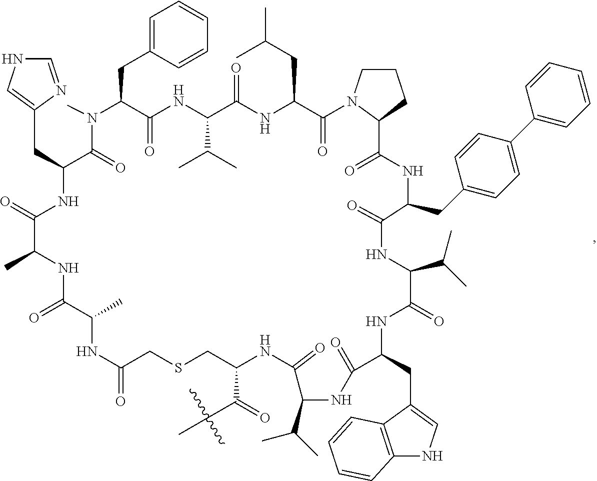 [SMILES: *C(=O)[C@@H]1CSCC(=O)N[C@@H](C)C(=O)N[C@@H](C)C(=O)N[C@@H](Cc2c[nH]cn2)C(=O)N(C)[C@@H](Cc2ccccc2)C(=O)N[C@@H](C(C)C)C(=O)N[C@@H](CC(C)C)C(=O)N2CCC[C@H]2C(=O)N[C@@H](Cc2ccc(-c3ccccc3)cc2)C(=O)N[C@@H](C(C)C)C(=O)N[C@@H](Cc2c[nH]c3ccccc23)C(=O)N[C@@H](C(C)C)C(=O)N1]
or a salt form thereof;
(b) (i) ABT has the structure of DCAWHLGELVWCT or a salt form thereof, wherein the two C residues are linked by a —S—S—, or (ii) ABT is or comprises optionally substituted 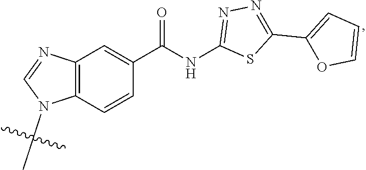 [SMILES: *n1cnc2cc(C(=O)Nc3nnc(-c4ccco4)s3)ccc21]
optionally substituted 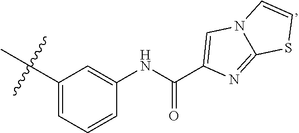 [SMILES: *c1cccc(NC(=O)c2cn3ccsc3n2)c1]
optionally substituted 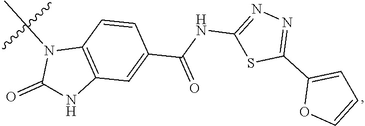 [SMILES: *n1c(=O)[nH]c2cc(C(=O)Nc3nnc(-c4ccco4)s3)ccc21]
optionally substituted 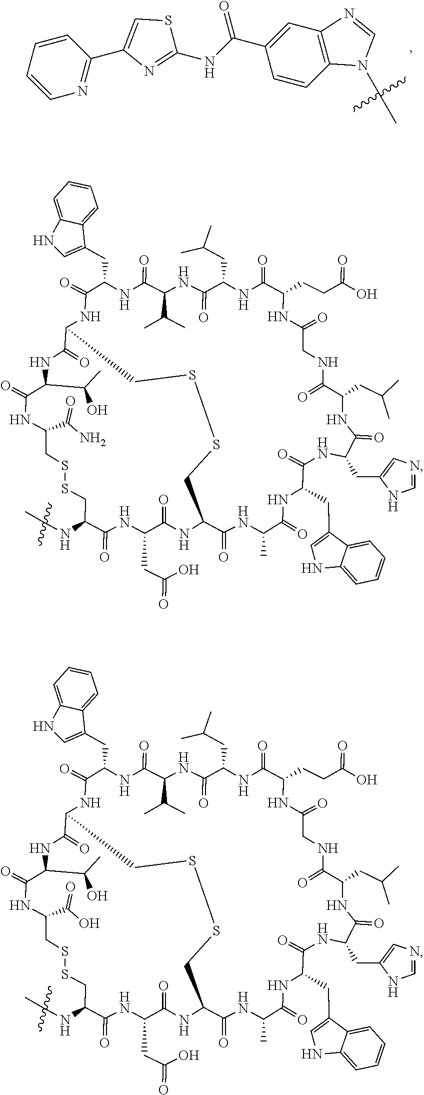 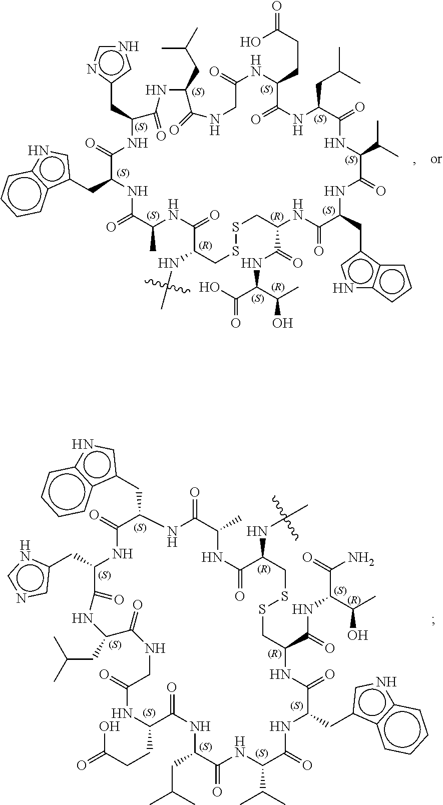
(c) the linker is or comprises (i) —(CH₂CH₂O)n-, wherein n is 1-20, (ii) one or more amino acid residues, 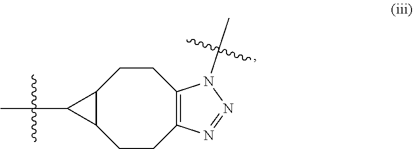 [SMILES: *C1C2CCc3nnn(*)c3CCC12]
or (iv) a combination thereof; or
(d) a combination thereof.

40. The agent of claim 3, wherein:
(a) TBT is or comprises a peptide whose sequence is or comprises a sequence selected from SEQ ID NO: 1-34, or
(ii) TBT is or comprises 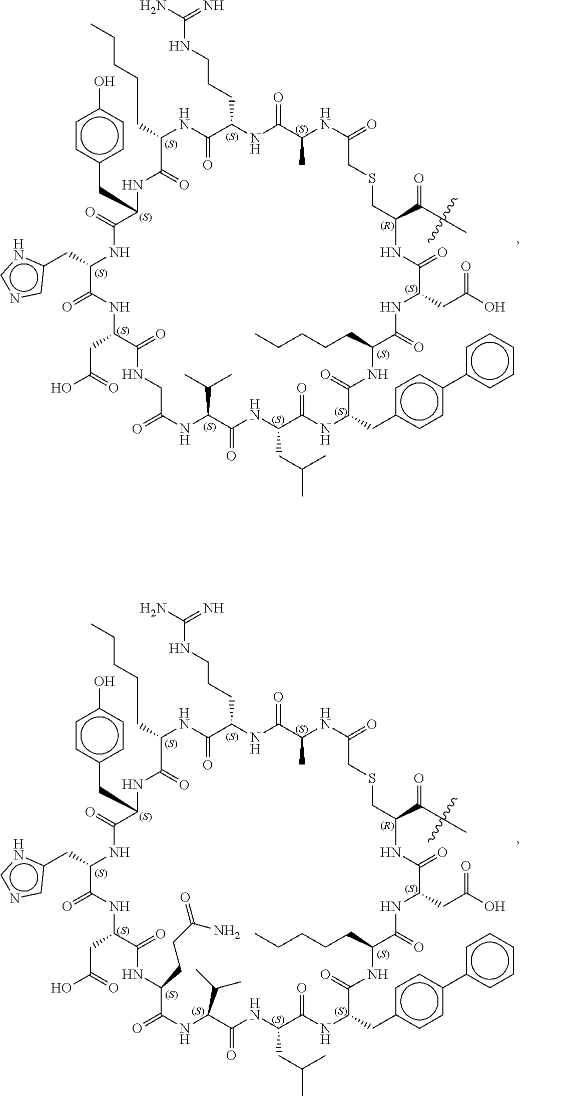 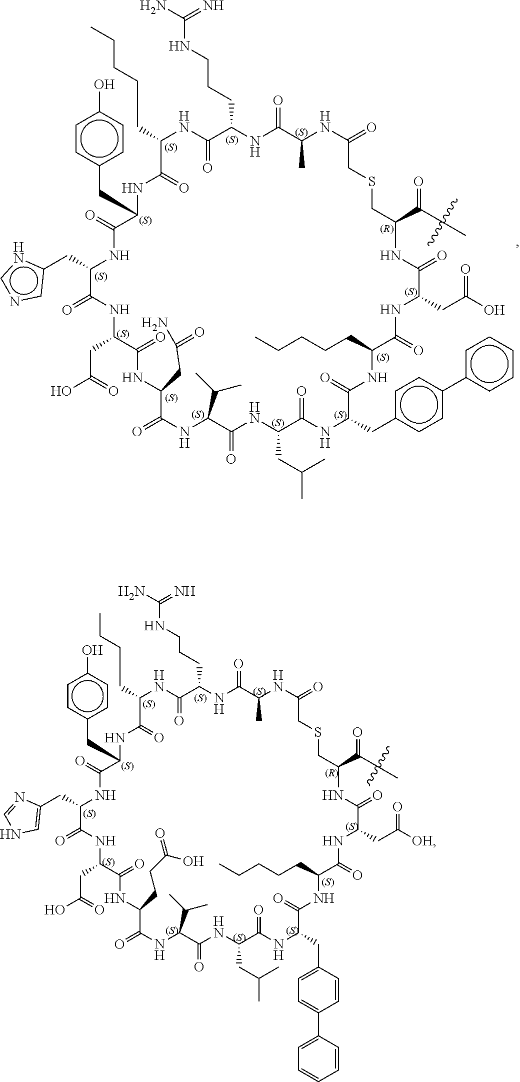 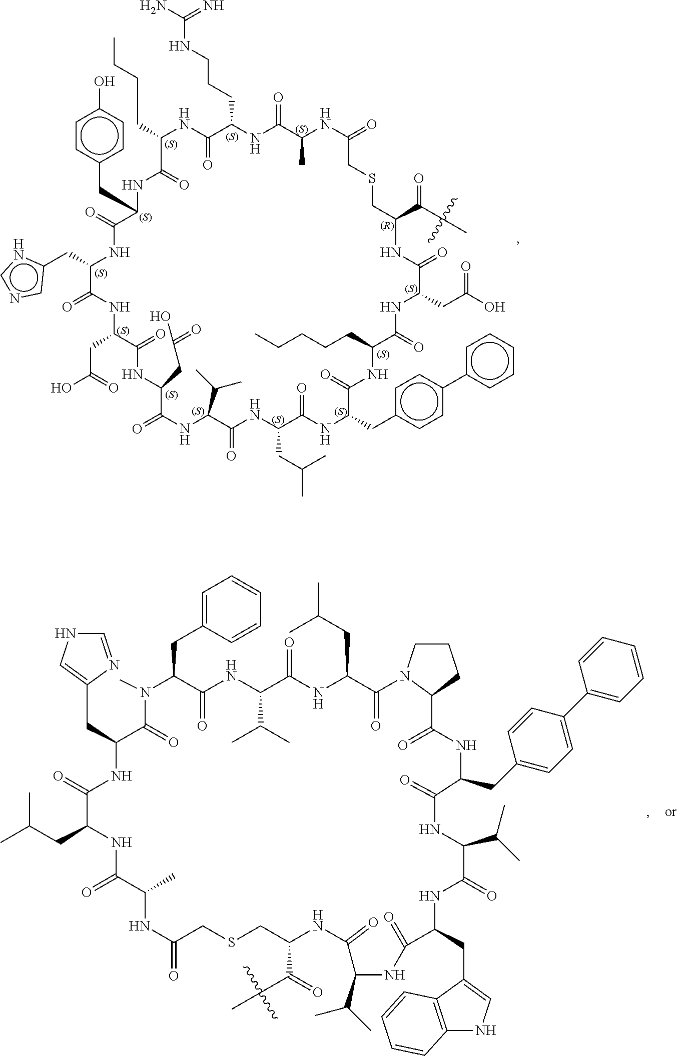 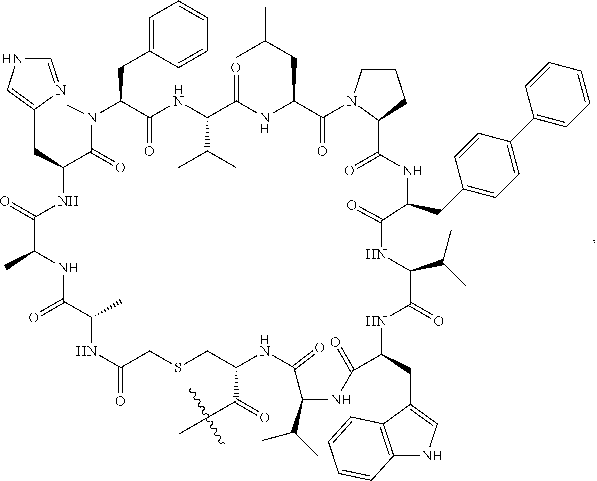 [SMILES: *C(=O)[C@@H]1CSCC(=O)N[C@@H](C)C(=O)N[C@@H](C)C(=O)N[C@@H](Cc2c[nH]cn2)C(=O)N(C)[C@@H](Cc2ccccc2)C(=O)N[C@@H](C(C)C)C(=O)N[C@@H](CC(C)C)C(=O)N2CCC[C@H]2C(=O)N[C@@H](Cc2ccc(-c3ccccc3)cc2)C(=O)N[C@@H](C(C)C)C(=O)N[C@@H](Cc2c[nH]c3ccccc23)C(=O)N[C@@H](C(C)C)C(=O)N1]
or a salt form thereof.

41. The agent of claim 3, wherein:
a) ABT has the structure of DCAWHLGELVWCT or a salt form thereof, wherein the two C residues are linked by a —S—S—; or
(b) ABT is or comprises optionally substituted 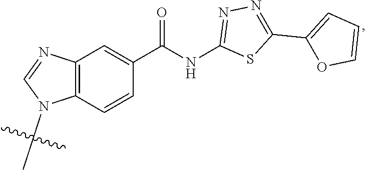 [SMILES: *n1cnc2cc(C(=O)Nc3nnc(-c4ccco4)s3)ccc21]
optionally substituted 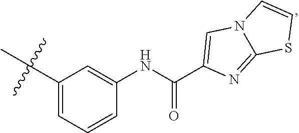 [SMILES: *c1cccc(NC(=O)c2cn3ccsc3n2)c1]
optionally substituted 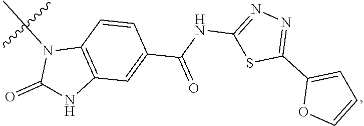 [SMILES: *n1c(=O)[nH]c2cc(C(=O)Nc3nnc(-c4ccco4)s3)ccc21]
optionally substituted

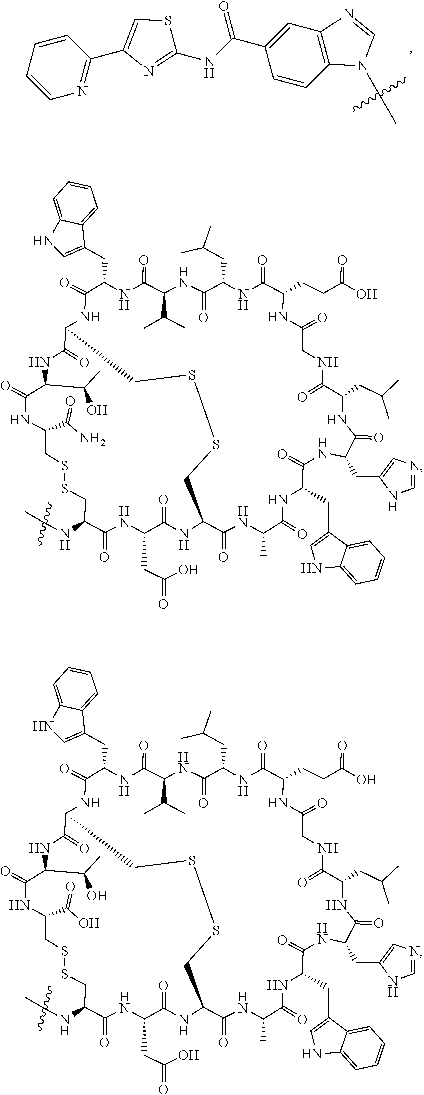

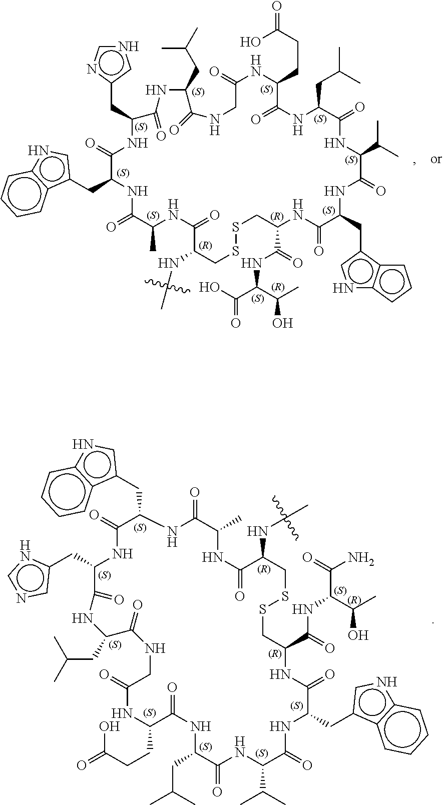

42. (canceled)

43. (canceled)

44. (canceled)

45. The agent of claim 3, wherein the linker is or comprises (i) —(CH₂CH₂O)n-, wherein n is 1-20, (ii) one or more amino acid residues, (iii) 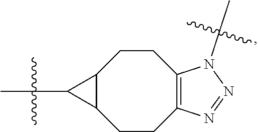 [SMILES: *C1C2CCc3nnn(*)c3CCC12]
or (iv) a combination thereof.

46. (canceled)

47. (canceled)

48. An agent, wherein the agent is I-1, I-2, I-3, I-4, I-5, I-6, I-7, I-8, I-9, I-10, I-11, I-12, I-13, I-14, I-15, I-16, I-17, I-18, I-19, I-24, I-25, I-26, I-27, I-28, I-29, I-30, I-31, I-32, I-33, I-34, I-35, I-36, I-37, I-38, I-39, I-40, I-41, I-42, I-43, I-44, I-45, I-46, or I-47, or pharmaceutically acceptable salt thereof.

49. (canceled)

50. A composition comprising an agent of claim 3 and a population of cells.

51. The composition of claim 50, wherein the cells are or comprise NK cells, engineered NK cells, NK cells expanded ex vivo, memory-like NK cells, cytokine-induced memory like NK cells, NKT cells, monocytes, and/or macrophages.

52. A pharmaceutical composition comprising an agent of claim 3 and a pharmaceutically acceptable carrier.

53. The composition of claim 52, further comprising an immunoglobulin.

54. (canceled)

55. A method for treating a CD38-associated condition, disorder, or disease, the method comprising administering to a subject suffering therefrom an effective amount of an agent of claim 3, optionally further comprising administering to the subject a population of cells, intravenous immunoglobulin, or both.

56. (canceled)

57. (canceled)

58. The method of claim 55, wherein the cells are or comprise NK cells, engineered NK cells, NK cells expanded ex vivo, memory-like NK cells, cytokine-induced memory like NK cells, NKT cells, monocytes, and/or macrophages.

59. (canceled)

60. (canceled)

61. (canceled)

62. (canceled)

63. (canceled)

64. The method of claim 55, wherein an administration of the agent is followed by one or more doses of the population of cells and/or one or more does of the population of cells and the agent.

65. (canceled)

66. (canceled)

67. (canceled)

68. (canceled)

69. A method for recruiting an antibody or immune activity to a target comprising or expressing CD38, the method comprising contacting the target with the agent of claim 3, optionally the target is a tumor cell.

70. (canceled)

71. (canceled)

72. (canceled)
